(19) United States
(12) Patent Application Publication
(10) Pub. No.: US 2008/0299102 A1
(43) Pub. Date: Dec. 4, 2008

(54) Novel BACE proteins, nucleic acid molecules therefor, novel crystal structure of novel BACE proteins, and methods for making and using

(76) Inventors: Jeff Yon, Cambridge (GB); Anne Cleasby, Cambridge (GB); Wouter David Bruinzeel, Beerse (BE); Stefan Leo Masure, Beerse (BE); Ian Tickle, Cambridge (GB); Andrew Sharff, Cambridge (GB)

Correspondence Address: FOLEY & LARDNER LLP, 111 HUNTINGTON AVENUE, 26TH FLOOR, BOSTON, MA, 02199-7610, US

(21) Appl. No.: 10/762,040

(22) Filed: Jan. 21, 2004

Related U.S. Application Data
(63) Continuation of application No. PCT/GB02/03461, filed on Jul. 26, 2002.
(60) Provisional application No. 60/308,366, filed on Jul. 26, 2001.

(51) Int. Cl. A61K 48/00; C12N 9/00; G01N 33/53; G06G 7/48; C07H 21/04; G06F 19/00; C12N 15/00; C07K 16/00; C12N 5/00

(52) U.S. Cl. 424/94.2; 435/183; 435/7.1; 702/19; 536/23.7; 435/320.1; 530/387.1; 435/375; 703/11

(57) ABSTRACT

Disclosed and claimed are novel BACE proteins, crystal structures thereof, nucleic acid molecules therefor, and methods for making and using and uses of the same, especially for ascertaining inhibitors of BACE; and thus, disclosed and claimed too are inhibitors of BACE and methods of making and using the same.

### FIELD OF THE INVENTION

This invention relates generally to structural studies of the soluble Beta-site APP cleaving enzyme (BACE) catalytic domain (e.g., the aspartyl protease domains of BACE) and the corresponding structural information obtained by X-ray crystallography.

Moreover, the present invention relates to any one or more of:

A catalytic domain of BACE, or a form of BACE that is suitable for crystallization with the correct disulphide bonding that eliminates the need for refolding and/or apo-BACE crystals which are BACE crystals with no ligand bound, regardless of the source of the BACE) and/or apo-BACE crystals which are capable of being soaked with ligand to give complexes and/or a crystalline form of BACE having crystals that are grown at or near the physiological pH of the enzyme such as between about pH 5.6 and about pH 5.8 and/or having a space group of C2 and cell dimensions of a=236.63 Å or 236.63 Å±standard deviation (0.2 Å) or 236.63 Å±cell variability of 3 Å, b=105.02 Å or 105.02 Å±standard deviation (0.2 Å) or 105.02 Å±cell variability of 3 Å, and c=62.59 Å or 62.59 Å standard deviation (0.2 Å) or 62.59 Å±cell variability of 3 Å and β=101.32° or 101.32°±standard deviation (0.2°) between 101° and 108° with the asymmetric unit of the crystal containing three copies of BACE (e.g., from growth in the presence of OM99-2) or cell dimensions a=238.3 Å or 238.3 Å±standard deviation (0.2 Å) or 238.3 Å±cell variability of 3 Å, b=107.4 Å+standard deviation (0.2 Å) or 107.4 Å±cell variability of 3 Å, and c=60.4 Å or 60.4 Å+standard deviation (0.2 Å) or 60.4 Å±cell variability of 3 Å and β=101.89° or 101.89°±standard deviation (0.2°) or between 101° and 108° (e.g., from crystals grown in the absence of OM99-2) and/or having an X-ray diffraction pattern corresponding to or resulting from any or all of the foregoing;

Apo-BACE crystals that can be soaked, e.g., with ligands such as inhibitory or modulatory ligands, to give complexes, such as protein-ligand complexes;

A crystalline form of BACE or a BACE that has an active site containing one or more ligands other than the natural substrate or the substrate that occurs naturally or physiologically within the active site or apo-BACE crystals with no ligand bound, regardless of the source of the BACE; for instance, for use in rational drug design, as well as methods for ligand screening and design by X-ray crystallography;

BACE proteins comprising, containing, having, consisting essentially of and/or consisting of amino acid sequences of the catalytic domain, advantageously amino acid sequences that crystallize to the crystalline structure or a structure that mimics the crystalline structure (included in the term “BACE proteins”)—such as those, when compared with other BACE proteins (such as Genbank accession P56817) have one or more of: a mutation at amino acid (“aa”) 153 for instance to prevent glycosylation or facilitate crystallization and/or the growth of ordered, well-diffracting crystals such as asparagine to glutamine, a mutation at aa 172 for instance to prevent glycosylation or facilitate crystallization and/or the growth of ordered, well-diffracting crystals such as asparagine to glutamine, a mutation at aa 223 for instance to prevent glycosylation or facilitate crystallization and/or the growth of ordered, well-diffracting crystals such as asparagine to glutamine, a mutation at aa 354 for instance to prevent glycosylation or facilitate crystallization and/or the growth of ordered, well-diffracting crystals such as asparagine to glutamine, and one or more truncations (e.g., a BACE extending from Thr 22 to Ser 453)—whereby such proteins can also optionally include one or more of: a tag such as a His tag (e.g., a HIS₆ tag) for instance to facilitate purification; a non-BACE signal sequence to facilitate or increase secretion of the protein into cell culture medium such as a baculovirus signal sequence for example the baculovirus gp67 signal sequence; and a tag such as a FLAG tag to allow differentiation of species arising from incomplete pro-peptide cleavage (and separation if required);

BACE proteins that have one or more mutations to prevent glycosylation or facilitate crystallization and/or the growth of ordered, well-diffracting crystals, such as with reference to Genbank accession P56817: a mutation at amino acid (“aa”) 153 for instance to prevent glycosylation or facilitate crystallization and/or the growth of ordered, well-diffracting crystals such as asparagine to glutamine, and/or a mutation at aa 172 for instance to prevent glycosylation or facilitate crystallization and/or the growth of ordered, well-diffracting crystals such as asparagine to glutamine, and/or a mutation at aa 223 for instance to prevent glycosylation or facilitate crystallization and/or the growth of ordered, well-diffracting crystals such as asparagine to glutamine, and/or a mutation at aa 354 for instance to prevent glycosylation or facilitate crystallization and/or the growth of ordered, well-diffracting crystals such as asparagine to glutamine, and one or more truncations (e.g., a BACE extending from Thr 22 to Ser 453);

BACE proteins that include one or more of: a tag such as a His tag (e.g., a HIS₆ tag) for instance to facilitate purification; a non-BACE signal sequence to facilitate or increase secretion of the protein into cell culture medium such as a baculovirus signal sequence for example the baculovirus gp67 signal sequence; and a tag such as a FLAG tag to allow differentiation of species arising from incomplete pro-peptide cleavage (and separation if required);

One or more nucleic acid molecules (e.g., an isolated nucleic acid molecule) encoding the BACE proteins or at least a functional portion thereof including any of the foregoing proteins and/or amino acid sequences and/or gene products comprising, containing, having, consisting essentially of and/or consisting of amino acid sequences of the catalytic domain, advantageously amino acid sequences that crystallize to the crystalline structure or a structure that mimics the crystalline structure including those having reduced GC content via silent mutations from nucleotide sequences derived from wild-type BACE that would also encode the foregoing;

Vectors or cells (e.g., viral vectors such as baculovirus, bacterial vectors such as E. coli, mammalian cells such as CHO cells, or DNA plasmids) containing and/or expressing any one or more of the nucleic acid molecules and/or BACE proteins—the latter can include prior BACE proteins especially when there is co-expression thereof with a gene product—an enhancer—that enhances in the particular vector or cell system the total amount of BACE produced and/or increases the fraction of processed protein such as an enzyme, for instance a convertase or a transcription enhancer or a translation enhancer or both a transcription and translation enhancer, for instance a prohormone convertase such as the prohormone convertase furin especially when the vector or cell system is baculovirus and/or insect cells, and thus also vectors or cells containing and/or expressing the nucleic acid molecules and/or BACE proteins and a nucleic acid molecule encoding the enhancer as well as kits containing separately packaged isolated nucleic acid molecules for such co-expression, e.g., a kit containing separately packaged nucleic acid molecules comprising (i) a BACE-protein encoding nucleic acid molecule and (ii) a nucleic acid molecule encoding the enhancer, for use in vectors or cells for the co-expression thereof;

Expression through or by vectors or cells of that which is encoded by the nucleic acid molecules and/or contained in the aforementioned vectors or cells and/or of the gene products and/or the amino acid sequences and/or the BACE proteins, including co-expression thereof, or of other nucleic acid molecules encoding BACE proteins, with a gene product that enhances in the particular vector or cell system the total amount of BACE produced and/or increases the fraction of processed protein such as an enzyme, e.g., a convertase, for instance a prohormone convertase such as the prohormone convertase furin especially when the vector or cell system is baculovirus and/or insect cells;

Methods for crystallizing BACE proteins and/or amino acid sequences and/or gene products comprising, containing, having, consisting essentially of and/or consisting of amino acid sequences of the catalytic domain;

Methods for determining the crystal structure of BACE proteins and/or amino acid sequences and/or gene products comprising, containing, having, consisting essentially of and/or consisting of amino acid sequences of the catalytic domain;

Uses of that which is encoded by the nucleic acid molecules and/or the gene products and/or the amino acid sequences and/or the BACE proteins, for instance in screening assays such as drug or patient screening assays or in generating products therefor (such as for generating antibodies to the catalytic domain and/or to BACE proteins which are useful in such assays), as well as such assays and products therefor, and uses of the nucleic acid molecules, vectors or cells, methods and/or the aforementioned expression via vectors or cells, for preparing such uses or assays and/or components for such uses or assays;

Products from such assays (“assay products”), as well as uses of the nucleic acid molecules, vectors or cells, methods and/or the aforementioned expression via vectors or cells for preparing such assay products and/or components for such assay products;

Inhibitors or modulators of BACE and/or inhibitors or modulators of the production of Aβ or fragments thereof for instance, such inhibitors or modulators as determined through the assays of the present invention and/or through contact with and binding to or otherwise inhibiting or modulating BACE proteins of the present invention, such as a compound or composition which binds to and/or inhibits and/or modulates and/or interacts with a form of BACE that is suitable for crystallization with the correct disulphide bonding that eliminates the need for refolding and/or having an unoccupied or substantially unoccupied active site and/or a crystalline form of BACE having crystals that are grown at or near the physiological pH of the enzyme such as between about pH 5.6 and about pH 5.8 and/or having a space group of C2 and cell dimensions of a=236.63 Å or 236.63 Å±standard deviation (0.2 Å) or 236.63 Å±cell variability of 3 Å, b=105.02 Å or 105.02 Å+standard deviation (0.2 Å) or 105.02 Å±cell variability of 3 Å, and c=62.59 Å or 62.59 Å+standard deviation (0.2 Å) or 62.59 Å±cell variability of 3 Å and 1101.32° or 101.32°±standard deviation (0.2°) or between 101° and 108° with the asymmetric unit of the crystal containing three copies of BACE (e.g., from growth in the presence of OM99-2) or cell dimensions a=238.3 Å or 238.3 Å+standard deviation (0.2 Å) or 238.3 Å±cell variability of 3 Å, b=107.4 Å±standard deviation (0.2 Å) or 107.4 Å±cell variability of 3 Å, and c=60.4 Å or 60.4 Å±standard deviation (0.2 Å) or 60.4 Å±cell variability of 3 Å and β=101.89° or 101.89°±standard deviation (0.2°) or between 101° and 108° (e.g., from crystals grown in the absence of OM99-2) and/or having an X-ray diffraction pattern corresponding to or resulting from any or all of the foregoing (excluding, of course, prior known inhibitors, modulators, if any, of BACE and/or inhibitors, modulators of the production of Aβ or fragments thereof);

Uses of such assay products and/or inhibitors and/or modulators, for instance in treating maladies, conditions, diseases and the like such as Alzheimer's disease (AD) involving BACE activity and/or Aβ or fragments thereof and/or in formulating medicaments for such treatments, as well as of uses of the nucleic acid molecules, vectors or cells, the methods and/or the aforementioned expression via vectors or cells, for such treatment and/or a component thereof and/or for preparing such medicaments and/or a component thereof, such that methods for preparing such medicaments including use of any of the foregoing is included, inter alia.

And a data storage medium encoded with the structural co-ordinates of crystallized BACE or at least a functional portion thereof. Such data storage material is capable of displaying such structures, or their structural homologues, as a graphical three-dimensional representation on a computer screen. This invention also relates to methods of using the structure co-ordinates to solve the structure of similar or homologous proteins or protein complexes. In addition, this invention relates to methods of using structure co-ordinates to screen and design compounds, including inhibitory compounds, that bind to BACE or homologues thereof. The present invention also relates to compositions and crystals of BACE in complex with a BACE inhibitor. Cf. WO 01/37194.

Various documents are cited in this text. Citations in the text can be by way of a citation to a document in the reference list, e.g., by way of author(s) and document year citation to a document listed in the reference list, or by full citation in the text to a document that may or may not also be listed in the reference list.

There is no admission that any of the various documents cited in this text are prior art as to the present invention. Any document having as an author or inventor person or persons named as an inventor herein is a document that is not by another as to the inventive entity herein.

All documents cited in this text (“herein cited documents”) and all documents cited or referenced in herein cited documents are hereby incorporated herein by reference. Likewise, teachings of herein cited documents and documents cited in herein cited documents can be employed in the practice and utilities of the present invention.

### BACKGROUND OF THE INVENTION

Alzheimer's disease (AD) is estimated to afflict more than 20 million people worldwide and is believed to be the most common form of dementia (Newsday (New York), Friday, Jul. 6, 2001, City Edition, page A24). AD is a progressive dementia characterized by amyloid plaques and intracellular neurofibrillary tangles that accumulate in the brain and are thought to be responsible for the mental decline in Alzheimer's patients.

Beta-amyloid protein (Aβ) is the major constituent of the amyloid plaques, which are characteristic of AD (De Strooper and Konig, 1999).

Aβ is a 39-42 amino acid residue peptide formed by the specific cleavage of a class I transmembrane protein called the amyloid precursor protein (APP) by two proteases, β- and γ-secretase (the Aβ fragment).

β-secretase cleaves APP between residues Met671 and Asp672 (numbering corresponds to the 770 amino acid isoform of APP) to form the N-terminus of Aβ. A second cleavage of the peptide is associated with γ-secretase to form the C-terminus of the Aβ peptide. β and γ-secretases cleave the amino and carboxy terminal ends of the Aβ domain, respectively. A third enzyme, α-secretase, has recently been identified which cleaves APP within the Aβ domain between residues 16 and 17 of the Aβ fragment (Howlett et al., 2000).

The therapeutic potential of inhibiting and/or modulating the deposition of Aβ has motivated many groups to isolate and characterize secretase enzymes and to identify their potential inhibitors (see, e.g., WO01/23533 A2, EP0855444A2, WO00/17369, WO00/58479, WO00/47618, WO01/00665; WO01/00663; U.S. Pat. No. 6,245,884 (Hook), U.S. Pat. No. 6,221,667 (Reiner et al.), U.S. Pat. No. 6,211,235 (Wu et al.)). Indeed, it also has been reported in the popular press that “[d]rug makers are studying medicines called gamma-secretase inhibitors, which aim to block the cleavage process” (Newsday (New York), Friday, Jul. 6, 2001, City Edition, page A24).

Consequently, a number of potential candidates for these enzymes have recently been reported in the literature: Several groups have identified and isolated aspartate proteases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000).

BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches has led to the identification of the same enzyme, making a strong case that BACE is a β-secretase. Mention is also made of the patent literature: WO91/13904, EP518955, EP732399, WO92/03542, WO92/07068, WO96/40885, EP87/1720, U.S. Pat. Nos. 5,942,400 and 5,744,346, EP855444, EP1037977, WO00/17369, WO01/23533, WO0047618, WO00/58479, WO01/00663, WO01/00665, EP848062, U.S. Pat. Nos. 6,025,180 and 6,162,639, EP1047788 and WO99/33963, WO99/46281, WO98/11236, U.S. Pat. No. 5,942,400 and WO94/13319.

Indeed, BACE is a membrane bound protein which is synthesized as a partially active proenzyme, and is most abundantly expressed in brain tissue. It is thought to represent the major β-secretase activity.

BACE activity may be considered to be a rate-limiting step in the production of Aβ. This makes BACE of special interest in the pathology of Alzheimer's disease and other maladies that involve Aβ, or fragments thereof (e.g., amyloid plaques and amyloid angiopathy also characterize the brains of individuals with Trisomy 21 or Down's Syndrome, Hereditary Cerebral Hemorrhage with Amyloidosis of the Dutch Type (HCHWA-D), inter alia; see also U.S. Pat. No. 6,211,235), and therefore an important candidate for the development of drugs as a treatment against Alzheimer's disease and/or against such other maladies.

Furthermore, as reported in the popular press, Newsday (New York), Friday, Jul. 6, 2001, City Edition, page A24, that day's edition of Science includes in vitro findings by investigator Thomas Sudhof of the Howard Hughes Medical Institute which suggest that gamma secretase may be implicated in another function, but that it is not known if those findings apply to humans or which genes may be involved. Nonetheless, inhibiting gamma secretase may have issues which are addressed by the present invention involving inhibiting BACE the production of Aβ or fragments thereof.

The likelihood of developing Alzheimer's disease increases with age, and as the aging population of the world increases, this disease may become a greater and greater problem. In addition, there is a familial link to AD and consequently any individuals possessing the double mutation of APP known as the Swedish mutation (in which the mutated APP forms a considerably improved substrate for BACE) have a much greater chance of developing AD and also of developing it at an early age (see also U.S. Pat. No. 6,245,964 pertaining to transgenic rodent comprising APP-Swedish).

It would therefore be useful to inhibit and/or modulate the deposition of Aβ and portions thereof; for instance by inhibiting and/or modulating BACE proteins through inhibitors or modulators thereof ascertained from BACE proteins having a particular crystal structure or having a structure as herein set forth.

Hence, drugs that reduce or block BACE activity would reduce Aβ levels and levels of fragments of Aβ in the brain or elsewhere where Aβ or fragments thereof deposit and thus slow the formation of amyloid plaques and the progression of AD or other maladies involving deposition of Aβ or fragments thereof (Yankner, 1996; De Strooper and Konig, 1999).

Further, reaction systems comprising Beta secretase have been asserted to be useful in screening assays, e.g., to identify inhibitors or modulators and antibodies raised against Beta-secretase have been asserted to be useful for screening and other assays; see, e.g., U.S. Pat. No. 6,221,645 and other documents cited herein; and thus, the present invention is likewise useful in such assays in generating antibodies.

There has been the production of certain active recombinant BACEs—different from those of the herein invention—using heterologous expression systems for mammalian cells (Vassar et al, 1999, Hassain et al, 1999), insect cells (Mallender et al, 2001) and bacterial cells (Lin et al 2000). While the production of these BACEs shows that no undue experimentation is needed to practice the present invention, these prior systems had deficiencies addressed by the herein invention.

Indeed, prior to the present invention there was a need to produce a soluble recombinant BACE protein with an improved crystal structure that is suitable for crystallization with the correct disulphide bonding that eliminates the need for refolding and/or having an unoccupied or substantially unoccupied active site and/or a crystalline form of BACE having crystals that are grown at or near the physiological pH of the enzyme such as between about pH 5.6 and about pH 5.8 and/or having a space group of C2 and cell dimensions of a=236.63 Å or 236.63 Å±standard deviation (0.2 Å) or 236.63 Å±cell variability of 3 Å, b=105.02 Å or 105.02 Å±standard deviation (0.2 Å) or 105.02 Å±cell variability of 3 Å, and c=62.59 Å or 62.59 Å±standard deviation (0.2 Å) or 62.59 Å±cell variability of 3 Å and β=101.32° or 101.32°±standard deviation (0.2°) or between 101° and 108° with the asymmetric unit of the crystal containing three copies of BACE (e.g., from growth in the presence of OM99-2) or cell dimensions a=238.3 Å or 238.3 Å±standard deviation (0.2 Å) or 238.3 Å±cell variability of 3 Å, b=107.4 Å±standard deviation (0.2 Å) or 107.4 Å±cell variability of 3 Å, and c=60.4 Å or 60.4 Å±standard deviation (0.2 Å) or 60.4 Å±cell variability of 3 Å and β=101.89° or 101.89°±standard deviation (0.2°) or between 101° and 108° (e.g., from crystals grown in the absence of OM99-2), as well as amino acid sequences therefor, nucleic acid molecules encoding them, and other aspects of the present invention as herein discussed.

In addition, the study of crystal structure and symmetry is developed (See, e.g., Cotton and Wilkinson, Inorganic Chemistry (John Wiley & Sons, Fourth Ed. 1980), especially Ch. 2). X-ray crystallography, or more generally crystallography, is an established, well-studied technique that provides what can best be described as a three-dimensional picture of what a molecule looks like in a crystal, and is useful for determining whether a compound that is not a known ligand of a target biomolecule can indeed bind as a ligand to a target biomolecule (see, e.g., WO 99/45379; U.S. Pat. No. 6,087,478; U.S. Pat. No. 6,110,672); and, there are additional techniques for identifying drug cores (see, e.g., WO 98/57155 regarding fragment-based screening). Mention is also made of U.S. Pat. Nos. 6,128,582, 6,153,579, 6,077,682, and 6,037,117 and PCT publications WO01/37194 and WO00/47763 for additional information on aspects of structure-based drug design and homology modelling.

These techniques can be employed with the herein disclosed BACE crystals and proteins, especially those that are without any ligands typically found in wild-type BACE, to rationally design compounds that inhibit or modulate, e.g., bind to or interact with BACE; and, the use of these techniques, in combination with herein disclosed BACE crystals and proteins it is believed has not been heretofore taught or suggested in the art.

### OBJECTS AND SUMMARY OF THE INVENTION

Without excluding inventions otherwise herein disclosed, the present invention can provide one or more of the following embodiments.

The present invention in an embodiment provides a catalytic domain of BACE, such as a form of BACE that is suitable for crystallization with the correct disulphide bonding that eliminates the need for refolding and/or a BACE protein having an unoccupied or substantially unoccupied active site (apo-BACE crystals with no ligand bound, regardless of the source of the BACE) and/or a crystalline form of BACE having crystals that are grown at or near the physiological pH of the enzyme such as between about pH 5.6 and about pH 5.8 and/or having a space group of C2 and cell dimensions of a=236.63 Å or 236.63 Å±standard deviation (0.2 Å) or 236.63 Å±cell variability of 3 Å, b=105.02 Å or 105.02 Å±standard deviation (0.2 Å) or 105.02 Å±cell variability of 3 Å, and c=62.59 Å or 62.59 Å±standard deviation (0.2 Å) or 62.59 Å±cell variability of 3 Å and β=101.32° or 101.32°±standard deviation (0.2°) or between 101° and 108° with the asymmetric unit of the crystal containing three copies of BACE (e.g., from growth in the presence of OM99-2) or cell dimensions a=238.3 Å or 238.3 Å±standard deviation (0.2 Å) or 238.3 Å±cell variability of 3 Å, b=107.4 Å or 107.4 Å±standard deviation (0.2 Å) or 107.4 Å±cell variability of 3 Å, and c=60.4 Å or 60.4 Å±standard deviation (0.2 Å) or 60.4 Å±cell variability of 3 Å and β=101.89° or 101.89°±standard deviation (0.2°) or between 101° and 108° (e.g., from crystals grown in the absence of OM99-2) and/or having an X-ray diffraction pattern corresponding to or resulting from any or all of the foregoing and/or having a space group transition from C2 to P2₁ together with an increase in the number of copies of the molecule in the asymmetric unit, while the cell dimensions and the packing of the P2₁ form are closely related to those of the C2 crystal form, on soaking the apo-BACE crystal with a ligand.

The present invention likewise provides apo-BACE crystals that can be soaked, e.g., with ligands such as inhibitory or modulatory ligands, to give complexes, such as protein-ligand complexes.

The present invention in another embodiment provides a crystalline form of BACE or a BACE that has an active site containing one or more ligands other than the natural substrate or the substrate that occurs naturally or physiologically within the active site or apo-BACE crystals with no ligand bound, regardless of the source of the BACE; for instance, for use in rational drug design, as well as methods for ligand screening and design by X-ray crystallography.

In regard to this, the invention further provides a method for ligand screening and/or design, e.g., by X-ray crystallography and/or nuclear magnetic resonance (NMR). The method can include exposing the apo crystals or BACE crystals with no ligand bound (i.e., with an unoccupied active site, regardless of the source of the BACE) to one or more test samples, and determining whether a ligand-BACE complex is formed, e.g., obtaining an X-ray crystal diffraction pattern to determine whether a ligand-BACE complex is formed or using NMR to determine whether such a complex is formed. The BACE can be exposed to the test samples by either co-crystallizing the BACE in the presence of the one or more test samples or soaking the BACE in a solution of one or more test samples. Structural information from ligand-BACE complexes can be used to design ligands that bind tighter, that bind more specifically, that have better biological activity or have a better safety profile. Cf. WO99/45379.

The present invention thus further provides a computer-assisted method for identifying or designing potential ligands to fit within the catalytic domain of BACE, using a programmed computer comprising a processor, a data storage system, an input device, and an output device, comprising the steps of: (a) inputting into the programmed computer through said input device data comprising the three-dimensional co-ordinates of a subset of the atoms in the BACE catalytic domain, e.g., BACE protein as herein provided and/or such information with structural information from ligand-BACE complexes, thereby generating a data set; (b) comparing, using said processor, said data set to a computer database of chemical structures stored in said computer data storage system; (c) selecting from said database, using computer methods, chemical structures having a portion that is structurally complementary to said data set; (d) optionally constructing, using computer methods, a model of a chemical structure having a portion that is structurally complementary to said data set and (e) outputting to said output device the selected chemical structures having a portion complementary to said data set; and optionally synthesizing one or more of the selected chemical structures; and further optionally contacting said synthesized selected chemical structure with BACE to ascertain whether said synthesized chemical structure is a ligand that fits within the catalytic domain of BACE and/or inhibits or modulates or interacts with BACE. Cf. U.S. Pat. No. 5,835,382.

In this way, one can rationally identify and/or design inhibitors or modulators of BACE or compounds that interact with BACE. And, in this regard, mention is made that the skilled artisan can employ the products found in the wild-type BACE catalytic domain as a portion of the information to be inputted or employed in the rational design and/or identification of inhibitors or modulators of BACE or compounds that interact with BACE. Furthermore, an inhibitor of BACE can be competitive, non-competitive, uncompetitive, or irreversible; and, inhibitors of BACE are of significant technical and commercial interest.

The present invention also provides BACE proteins comprising, containing, having, consisting essentially of and/or consisting of amino acid sequences of the catalytic domain, advantageously amino acid sequences that crystallize to the crystalline structure or a structure that mimics the crystalline structure (included in the term “BACE proteins”)—such as those, when compared with other BACE proteins (such as Genbank accession P56817) have one or more of: a mutation at amino acid (“aa”) 153 for instance to prevent glycosylation or facilitate crystallization and/or the growth of ordered, well-diffracting crystals such as asparagine to glutamine, a mutation at aa 172 for instance to prevent glycosylation or facilitate crystallization and/or the growth of ordered, well-diffracting crystals such as asparagine to glutamine, a mutation at aa 223 for instance to prevent glycosylation or facilitate crystallization and/or the growth of ordered, well-diffracting crystals such as asparagine to glutamine, a mutation at aa 354 for instance to prevent glycosylation or facilitate crystallization and/or the growth of ordered, well-diffracting crystals such as asparagine to glutamine, and one or more truncations (e.g., a BACE extending from Thr 22 to Ser 453)—whereby such proteins can also optionally include one or more of: a tag such as a His tag (e.g., a HIS6 tag) for instance to facilitate purification (cf. U.S. Pat. No. 6,020,143); a non-BACE signal sequence to facilitate or increase secretion of the protein into cell culture medium such as a baculovirus signal sequence for example the baculovirus gp67 signal sequence (cf. U.S. Pat. Nos. 6,245,532, 5,516,657); and a tag such as a HA or FLAG tag to allow differentiation of species arising from incomplete pro-peptide cleavage (and separation if required) (cf. U.S. Pat. Nos. 6,190,874, 6,083,732).

The present invention thus further provides BACE proteins that have one or more mutations to prevent glycosylation or facilitate crystallization and/or the growth of ordered, well-diffracting crystals, such as with reference to Genbank accession P56817: a mutation at amino acid (“aa”) 153 for instance to prevent glycosylation or facilitate crystallization and/or the growth of ordered, well-diffracting crystals such as asparagine to glutamine, and/or a mutation at aa 172 for instance to prevent glycosylation or facilitate crystallization and/or the growth of ordered, well-diffracting crystals such as asparagine to glutamine, and/or a mutation at aa 223 for instance to prevent glycosylation or facilitate crystallization and/or the growth of ordered, well-diffracting crystals such as asparagine to glutamine, and/or a mutation at aa 354 for instance to prevent glycosylation or facilitate crystallization and/or the growth of ordered, well-diffracting crystals such as asparagine to glutamine, and one or more truncations (e.g., a BACE extending from Thr 22 to Ser 453).

Advantageously, the BACE protein has all four of the mutations and the truncation.

The present invention additionally provides BACE proteins that include one or more of: a tag such as a His tag (e.g., a HIS₆ tag) for instance to facilitate purification; a non-BACE signal sequence to facilitate or increase secretion of the protein into cell culture medium such as a baculovirus signal sequence for example the baculovirus gp67 signal sequence; and a tag such as a FLAG tag to allow differentiation of species arising from incomplete pro-peptide cleavage (and separation if required).

Even further still, the present invention provides one or more nucleic acid molecules (e.g., an isolated nucleic acid molecule) encoding the BACE proteins or at least a functional portion thereof including any of the foregoing proteins and/or amino acid sequences and/or gene products comprising, containing, having, consisting essentially of and/or consisting of amino acid sequences of the catalytic domain, advantageously amino acid sequences that crystallize to the crystalline structure or a structure that mimics the crystalline structure including those having reduced GC content via silent mutations from nucleotide sequences derived from wild-type BACE that would also encode the foregoing.

In yet further embodiments, the present invention provides vectors or cells (e.g., viral vectors such as baculovirus, bacterial vectors such as E. coli, mammalian cells such as CHO cells, or DNA plasmids) containing and/or expressing any one or more of the nucleic acid molecules and/or BACE proteins—the latter can include prior BACE proteins especially when there is co-expression thereof with a gene product—an enhancer—that enhances in the particular vector or cell system, the total amount of BACE produced and/or increases the fraction of processed protein such as an enzyme e.g., a convertase, or a transcription enhancer or a translation enhancer or both a transcription and translation enhancer (cf. U.S. Pat. Nos. 6,130,066, 6,004,777, 5,990,091), for instance a prohormone convertase such as the prohormone convertase furin (cf. Laprise et al. 1998) when the vector or cell system is baculovirus and/or insect cells, and thus also vectors or cells containing and/or expressing the nucleic acid molecules and/or BACE proteins and a nucleic acid molecule encoding the enhancer as well as kits containing separately packaged isolated nucleic acid molecules for such co-expression, e.g., a kit containing separately packaged nucleic acid molecules comprising (i) a BACE-protein encoding nucleic acid molecule and (ii) a nucleic acid molecule encoding the enhancer, for use in vectors or cells for the co-expression thereof;

The invention thus also provides expression through or by vectors or cells of that which is encoded by the nucleic acid molecules and/or contained in the aforementioned vectors or cells and/or of the gene products and/or the amino acid sequences and/or the BACE proteins, including co-expression thereof, or of other nucleic acid molecules encoding BACE proteins, with a gene product that enhances in the particular vector or cell system the total amount of BACE produced and/or increases the fraction of processed protein such as an enzyme, e.g., a convertase, for instance a prohormone convertase such as the prohormone convertase furin especially when the vector or cell system is baculovirus and/or insect cells.

As the invention involves a unique crystal structure of BACE, the invention provides methods for crystallizing BACE proteins and/or amino acid sequences and/or gene products comprising, containing, having, consisting essentially of and/or consisting of amino acid sequences of the catalytic domain.

Similarly, the invention provides methods for determining the crystal structure of BACE proteins and/or amino acid sequences and/or gene products comprising, containing, having, consisting essentially of and/or consisting of amino acid sequences of the catalytic domain.

The invention further contemplates uses of that which is encoded by the nucleic acid molecules and/or the gene products and/or the amino acid sequences and/or the BACE proteins, for instance in screening assays such as drug or patient screening assays or in generating products therefor (such as for generating antibodies to the catalytic domain and/or to BACE proteins which are useful in such assays), as well as such assays and products therefor, and uses of the nucleic acid molecules, vectors or cells, methods and/or the aforementioned expression via vectors or cells, for preparing such uses or assays and/or components for such uses or assays.

Included within the ambit of the present invention are products from such assays (“assay products”), as well as uses of the nucleic acid molecules, vectors or cells, methods and/or the aforementioned expression via vectors or cells for preparing such assay products and/or components for such assay products.

The BACE protein of the present invention may be employed in screening for compounds which inhibit or modulate or activate or interact with this protein. Such compounds may be identified from cells or cell fractions, mixtures of natural products or chemical libraries.

The assay may comprise mixing the BACE polypeptide of the invention with a candidate compound in solution and measuring BACE activity in the mixture. It may also be advantageous to measure binding of the compound to the BACE polypeptide (or competition with binding of a known inhibitor) instead of an effect on enzyme activity. Alternatively, versions of the BACE protein containing the transmembrane region may be expressed in cells, and these cells (or membranes prepared from these cells) may be incubated with candidate compounds. The effect on BACE activity may then be assessed by measurement of cleavage of a suitable substrate, either added to the mixture or co-expressed in the cells.

The protein or antibodies to the protein may also be used to identify receptors, through standard techniques. These include, but are not limited to, ligand binding or cross-linking assays in which the BACE protein is labeled and contacted with a source of the putative receptor, and biophysical techniques such as surface plasmon resonance.

The present invention even further contemplates inhibitors or modulators of BACE or compounds or compositions that interact with BACE and/or inhibitors or modulators of the production of Aβ or fragments thereof, for instance, such inhibitors as determined through the assays of the present invention and/or through contact with and binding to or otherwise interacting with, inhibiting or modulating BACE proteins of the present invention, such as a compound or composition or ligand which binds to and/or inhibits and/or interacts with and/or modulates a form of BACE that is suitable for crystallization with the correct disulphide bonding that eliminates the need for refolding and/or having an unoccupied or substantially unoccupied active site and/or a crystalline form of BACE having crystals that are grown at or near the physiological pH of the enzyme such as between about pH 5.6 and about pH 5.8 and/or having a space group of C2 and cell dimensions of a=236.63 Å or 236.63 Å±standard deviation (0.2 Å) or 236.63 Å±cell variability of 3 Å, b=105.02 Å or 105.02 Å±standard deviation (0.2 Å) or 105.02 Å±cell variability of 3 Å, and c=62.59 Å or 62.59 Å±standard deviation (0.2 Å) or 62.59 Å±cell variability of 3 Å and β=101.32° or 101.32°±standard deviation (0.2°) or between 101° and 108° with the asymmetric unit of the crystal containing three copies of BACE (e.g., from growth in the presence of OM99-2) or cell dimensions a=238.3 Å or 238.3 Å±standard deviation (0.2 Å) or 238.3 Å±cell variability of 3 Å, b=107.4 Å±standard deviation (0.2 Å) or 107.4 Å±cell variability of 3 Å and c=60.4 Å or 60.4 Å+standard deviation (0.2 Å) or 60.4 Å±cell variability of 3 Å and β=101.89°, or 101.89°±standard deviation (0.2°) or between 101° and 108° (e.g., from crystals grown in the absence of OM99-2) and/or having an X-ray diffraction pattern corresponding to or resulting from any or all of the foregoing (excluding, of course, prior known inhibitors and modulators of BACE and/or inhibitors or modulators of the production of Aβ or fragments thereof).

And, the present invention provides uses of such assay products and/or inhibitors and/or modulators and/or ligands, and/or compositions or compounds that interact with BACE, for instance in treating maladies, conditions, diseases and the like such as Alzheimer's disease (AD) involving BACE activity and/or Aβ or fragments thereof and/or in formulating medicaments for such treatments, as well as of uses of the nucleic acid molecules, vectors or cells, the methods and/or the aforementioned expression via vectors or cells, for such treatment and/or a component thereof and/or for preparing such medicaments and/or a component thereof, such that methods for preparing such medicaments including use of any of the foregoing is included.

In a further embodiment, the present invention provides a Beta-site APP cleaving enzyme which comprises an amino acid sequence of SEQ ID NO: 5; advantageously, the amino acid sequence comprises a catalytic domain, and wherein the enzyme is in a crystalline form, such as herein defined.

In another embodiment the recombinant Beta-site APP cleaving enzyme comprises an amino acid sequence of SEQ ID NO: 5 (FIGS. 1B, 2A, 8), as well as nucleic acid molecules encoding such an enzyme; for instance, a nucleic acid molecule comprising a sequence of SEQ ID NO: 4 or 10 (FIGS. 1A, 2B, 7).

More in particular, with respect to the herein mentioned nucleic acid molecules and polypeptides therefrom, e.g., the aforementioned nucleic acid molecules (FIGS. 2B, 7) and polypeptides expressed from them (FIGS. 2A, 8), the invention further comprehends isolated and/or purified nucleic acid molecules and isolated and/or purified polypeptides having at least about 70%, preferably at least about 75% or about 77% identity or homology (“substantially homologous or identical”), advantageously at least about 80% or about 83%, such as at least about 85% or about 87% homology or identity (“significantly homologous or identical”), for instance at least about 90% or about 93% identity or homology (“highly homologous or identical”), more advantageously at least about 95%, e.g., at least about 97%, about 98%, about 99% or even about 100% identity or homology (“very highly homologous or identical” to “identical”; or from about 84-100% identity considered “highly conserved”); and advantageously these polypeptides obtain crystal structures as herein disclosed and the nucleic acid molecules encode polypeptides that obtain crystal structures as herein disclosed. Moreover, it is advantageous that polypeptides of the invention have greater than 98.8% identity to herein disclosed sequences, and that nucleic acid molecules of the invention have greater than 95.6% identity to herein disclosed sequences, especially as certain amino acid sequences of the invention have 98.8% identity to sequence 32 of WO01/23533 and certain nucleic acid molecules of the invention have 95.6% identity to sequence 25 of WO01/23533 (and it is intended to exclude any prior sequences). The invention also comprehends that these nucleic acid molecules and polypeptides can be used in the same fashion as the herein or aforementioned nucleic acid molecules and polypeptides.

Nucleotide sequence homology can be determined using the “Align” program of Myers and Miller, (“Optimal Alignments in Linear Space”, CABIOS 4, 11-17, 1988, incorporated herein by reference) and available at NCBI. Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence similarity of 75% with the sequence AATCAATC (N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur and Lipman, 1983, PNAS, USA 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence (see also alignment used in Figures).

RNA sequences within the scope of the invention can be derived from DNA sequences, by thymidine (T) in the DNA sequence being considered equal to uracil (U) in RNA sequences.

Additionally or alternatively, amino acid sequence similarity or identity or homology can be determined using the BlastP program (Altschul et al., Nucl. Acids Res. 25, 3389-3402 (1997), incorporated herein by reference) and available at NCBI. The following references (each incorporated herein by reference) provide algorithms for comparing the relative identity or homology of amino acid residues of two proteins, and additionally or alternatively with respect to the foregoing, the teachings in these references can be used for determining percent homology or identity: Needleman S B and Wunsch C D, “A general method applicable to the search for similarities in the amino acid sequences of two proteins,” J. Mol. Biol. 48:444-453 (1970); Smith T F and Waterman M S, “Comparison of Bio-sequences,” Advances in Applied Mathematics 2:482-489 (1981); Smith T F, Waterman M S and Sadler J R, “Statistical characterization of nucleic acid sequence functional domains,” Nucleic Acids Res., 11:2205-2220 (1983); Feng D F and Dolittle R F, “Progressive sequence alignment as a prerequisite to correct phylogenetic trees,” J. of Molec. Evol., 25:351-360 (1987); Higgins D G and Sharp P M, “Fast and sensitive multiple sequence alignment on a microcomputer,” CABIOS, 5: 151-153 (1989); Thompson J D, Higgins D G and Gibson T J, “ClusterW: improving the sensitivity of progressive multiple sequence alignment through sequence weighing, positions-specific gap penalties and weight matrix choice, Nucleic Acid Res., 22:4673-480 (1994); and, Devereux J, Haeberlie P and Smithies O, “A comprehensive set of sequence analysis program for the VAX,” Nucl. Acids Res., 12: 387-395 (1984).

In this fashion, by comprehending nucleic acid molecules and polypeptides having such homology to the particular sequences disclosed, it is envisioned that the invention encompasses homologues to the disclosed sequences, within the herein terms.

As to homologues of the disclosed amino acid sequences (FIGS. 2A, 8), it is advantageous that these homologues have the herein defined crystal structure; and, as to homologues of the disclosed nucleic acid sequences, it is advantageous that these homologues encode BACE proteins having the herein defined crystal structure.

Furthermore, as to inventive nucleic acid molecules, the invention comprehends codon equivalent nucleic acid molecules. For instance, if the invention comprehends “X” protein having amino acid sequence “A” and nucleic acid molecule “N” encoding protein X, the invention comprehends nucleic acid molecules that also encode protein X via one or more different codons than in nucleic acid molecule N.

In addition, as to inventive nucleic acid molecules, the invention comprehends nucleic acid molecules that hybridize under stringent conditions to herein disclosed nucleic acid molecules.

As to herein disclosed amino acid sequences, the invention comprehends nucleic acid molecules encoding the herein disclosed amino acid sequences, as well as nucleic acid molecules that hybridize under stringent conditions to nucleic acid molecules encoding herein disclosed amino acid sequences, as these nucleic acid molecules that hybridize under stringent conditions to nucleic acid molecules encoding herein disclosed amino acid sequences can provide proteins having similarity, homology or identity as herein discussed, especially if the proteins have the same or substantially the same crystal structure as herein disclosed.

The present invention further provides in particular embodiments a crystalline structure of both the soluble BACE catalytic domain in the presence of OM99-2 and in the absence of OM99-2, both having a space group of C2 and/or having an X-ray diffraction pattern corresponding to or resulting from any or all of the foregoing and/or having a space group transition from C2 to P2, together with an increase in the number of copies of the molecule in the asymmetric unit, while the cell dimensions and the packing of the P2, form are closely related to those of the C2 crystal form, on soaking the apo-BACE crystal with a ligand The cell dimensions of the crystals grown in the presence of OM99-2 (FIG. 3A) are a=236.63 Å, b=105.02 Å, and c=62.59 Å and β=101.32° and the asymmetric unit of the crystal containing three copies of BACE. The cell dimensions of the crystals grown in the absence of OM99-2 (FIG. 3B) are a=−238.3 Å, b=107.4 Å, and c=60.4 Å and β=101.89°. However, as is evident from the present disclosure, the invention is not limited by the crystals having been grown in the presence or absence of OM99-2 or anything else, and that cell dimensions can vary in all directions of the cell dimensions from a stated value, e.g., a stated cell dimension value can be that value±standard deviation (0.2 Å) or ±cell variability of 3 Å, and that the stated beta angle can vary, e.g., a stated beta angle can be that value, for instance 101.32° or 101.89° or that value±standard deviation (0.2°) or between 101° and 108°.

BACE crystals of the present invention can have a resolution better than, i.e., numerically lower than 3 Å.

The present invention further provides a method of employing the crystals of the present invention in drug screening assays, comprising selecting a potential compound which binds to the active site of the BACE catalytic domain of BACE, as well as to uses of such a compound, as herein mentioned.

The present invention further provides a data storage medium encoded with the structural co-ordinates of crystallized BACE or at least a functional portion thereof. Such data storage material is capable of displaying such structures, or their structural homologues, as a graphical three-dimensional representation on a computer screen. This invention also relates to methods of using the structure co-ordinates to solve the structure of similar or homologous proteins or protein complexes. In addition, this invention relates to methods of using structure co-ordinates to screen and design compounds, including inhibitory compounds, that bind to BACE or homologues thereof. The present invention also relates to compositions and crystals of BACE in complex with a BACE inhibitor. Cf. WO 01/37194.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

These and other embodiments are disclosed or are obvious from and encompassed by, the following Detailed Description.

### BRIEF DESCRIPTION OF FIGURES

The following Detailed Description, given to describe the invention by way of example, but not intended to limit the invention to specific embodiments described, may be understood in conjunction with the accompanying Figures, incorporated herein by reference, in which:

FIG. 1A shows an alignment of BACE DNA sequences (EMBL-AF190725.SEQ, EMBL-AF200343.SEQ, and EMBL-AF204943.SEQ), and a BACE DNA sequence of the present invention (BACE_dna.SEQ) (SEQ ID NOs: 1-4), illustrating the novelty, nonobviousness and inventive step of the present invention*, **;

FIG. 1B shows an alignment of a BACE polypeptide sequence of the present invention (baceprot.pro) and a BACE polypeptide sequence (P56817.pro) (SEQ ID NOs: 5-6), illustrating the novelty, nonobviousness and inventive step of the present invention*, **;

FIG. 2A shows an inventive BACE polypeptide sequence encoded by a BACE nucleotide sequence of the present invention (SEQ ID NO: 5);

FIG. 2B shows an inventive BACE nucleotide sequence (SEQ ID NO: 4);

FIG. 3A shows a photograph from a light microscope of the BACE crystal grown in the presence of OM99-2;

FIG. 3B shows a photograph from a light microscope of the BACE crystal grown in the absence of any added inhibitor (OM99-2);

FIG. 4A shows a diagram providing the arrangement of BACE monomers in asymmetric unit of crystallographic cell (The blue (molecule C) and orange (molecule B) molecules of the dimer, which is homologous to the dimer of Tang et al. WO01/00663, Tang et al. WO01/00665, Hong et al., Science, 2000; 290, 150-153; the molecule in pink (molecule A) forms a dimer with a crystallographically related molecule, which is homologous to the non-crystallographic dimer);

FIG. 4B shows a diagram providing the packing of the molecules in the unit cell of BACE (The pink (C), orange (B) and blue molecules (A) form the asymmetric unit, which is related to the molecules in red (D), dark blue (E) and green (F) by crystallographic symmetry);

FIG. 5 shows a copy of the gel from SDS-PAGE purification of BACE;

FIG. 6 shows a diagrammatic representation of the comparison between the BACE protein of the present invention versus Tang et al. WO01/00663, Tang et al. WO01/00665, Hong et al., Science, 2000; 290, 150-153 (the downward facing arrows are the sites of proteolytic cleavage; TM is the transmembrane region and cyt is the cytoplasmic region), illustrating the novelty and nonobviousness and inventive step of the present invention;

FIG. 7 shows an alignment of BACE DNA sequences (e.g., Ep855444.seq, WO100663.SEQ, and WO0123533seq25.SEQ) and a BACE DNA sequence of the present invention (BACE_dna.SEQ) (SEQ ID NOs: 7-9 and 4), illustrating the novelty and nonobviousness and inventive step of the present invention*, ***;

FIG. 8 shows an alignment of BACE amino acid sequences (e.g., WO0123533SEQ32.pro and WO0100663.PRO) and a BACE amino acid sequence of the present invention (baceprot.pro) (SEQ ID NOs: 10-11, and 5), illustrating the novelty and nonobviousness and inventive step of the present invention*, ***;

(* Figure color coded to show similarities and/or differences.)

(** EMBL-AF190725.SEQ, EMBL-AF200343.SEQ, and EMBL-AF204943.SEQ are EMBL sequences; P56817.pro is a Genbank sequence, accession P56817.)

(*** Ep855444.seq, WO0100663.SEQ, WO0123533seq25.SEQ, WO0123533SEQ32.pro and WO0100663.PRO are sequences from European Patent Application 855444, and PCT publications WO01/00663, WO01/23533, WO01/23533 and WO01/00663.)

### DETAILED DESCRIPTION

The present invention involves a catalytic domain of BACE, or a form of BACE that is suitable for crystallization with the correct disulphide bonding. Correct disulphide bonding refers to the disulphide bonding of a biologically active conformation of a catalytic domain of BACE or a BACE protein that retains functionality. Having the correct disulphide bonding eliminates the need for refolding and/or a catalytic domain of BACE or a BACE protein having an unoccupied or substantially unoccupied active site (apo-BACE crystals with no ligand bound, regardless of the source of the BACE) and/or a crystalline form of BACE having crystals that are grown at or near the physiological pH of the enzyme such as between about pH 5.6 and about pH 5.8 and/or having a space group of C2 and cell dimensions of a=236.63 Å or 236.63 Å±standard deviation (0.2 Å) or 236.63 Å±cell variability of 3 Å, b=105.02 Å or 105.02 Å±standard deviation (0.2 Å) or 105.02 Å±cell variability of 3 Å, and c=62.59 Å or 62.59 Å±standard deviation (0.2 Å) or 62.59 Å±cell variability of 3 Å and β=101.32° or 101.32°±standard deviation (0.2°) or between 101° and 108° with the asymmetric unit of the crystal containing three copies of BACE (e.g., from growth in the presence of OM99-2) or cell dimensions a=238.3 Å or 238.3 Å±standard deviation (0.2 Å) or 238.3 Å±cell variability of 3 Å, b=107.4 Å±standard deviation (0.2 Å) or 107.4 Å±cell variability of 3 Å, and c=60.4 Å or 60.4 Å±standard deviation (0.2 Å) or 60.4 Å±cell variability of 3 Å and β=101.89° or 101.89°±standard deviation (0.2°) or between 101° and 108° (e.g., from crystals grown in the absence of OM99-2) and/or having an X-ray diffraction pattern corresponding to or resulting from any or all of the foregoing and/or having a space group transition from C2 to P2₁ together with an increase in the number of copies of the molecule in the asymmetric unit, while the cell dimensions and the packing of the P2₁ form are closely related to those of the C2 crystal form, on soaking the apo-BACE crystal with a ligand.

The present invention further involves the expression of these BACE proteins and their use; for instance in the rational design or identification of inhibitors or modulators of BACE.

The BACE recombinant proteins of the present invention are advantageously expressed in insect cells through a baculovirus expression system and are soluble and lack glycosylation. Increased solubility can be achieved by C-terminal truncation of the protein to remove the transmembrane and cytoplasmic regions, while glycosylation can be removed by introducing mutations at the glycosylation sites. WO01/00663 (Tang et al.), WO01/00665 (Tang et al.), Hong et al., Science, 2000; 290, 150-153, in contrast, produced the C-terminally truncated memapsin 2 protein in bacteria for crystallization. More specifically, memapsin 2 was produced as insoluble inclusion bodies in bacteria. Therefore refolding was necessary to give a soluble, active protein. However during refolding/purification the N-terminal region was lost, due to unidentified proteolytic activity. Furthermore the final protein used for crystallization studies was a mixture of species, the majority having an N-terminus at Leu41 and a minority at Leu43 (the mature N-terminus is at Glu46). See Table 4, infra, for a comparison of the Tang/Hong crystal structure with the present invention.

The exemplified BACE protein was expressed with: 1) a His₆ tag added at the C-terminus to facilitate purification; 2) mutations of the asparagine residue to glutamine in the four potential glycosylation sites at amino acids 153, 172, 223 and 354, to prevent glycosylation of the protein; 3) an N-terminus generated by furin cleavage; 4) a FLAG oligopeptide tag added to the N-terminus of the pro-peptide to enable differentiation between processed and unprocessed protein and 5) a signal peptide derived from the gp67 baculoviral protein.

Possible vectors for use in the present invention, e.g., for expressing BACE or a nucleic acid molecule encoding BACE, include, but are not limited to: for insect cells, pFastBAc1 (Life Technologies), pFastBAcDual pFastBAc1 (Life Technologies), pBlueBac III or pBlueBacHis baculovirus vectors (Invitrogen, San Diego, Calif.); for bacterial cells, pET-3 (Novagen, Madison, Wis.) and for mammalian cells, pJT4 (discussed further below), pcDNA-1 (Invitrogen, San Diego, Calif.) and pSV-SPORT 1 (Gibco-BRL, Gaithersburg, Md.). Thus, any suitable vector can be used for expression of the BACE catalytic domain or proteins or for replication and/or expression of nucleic acid molecules of the invention, including e.g., in bacterial systems such as Escherichia coli, or in viral vector systems, and DNA plasmid systems. The methods for making a vector or recombinant or plasmid for expression of BACE or nucleic acid molecules encoding BACE can be any desired method, e.g., a method which is by or analogous to the methods disclosed herein cited documents and/or in: U.S. Pat. Nos. 4,603,112, 4,769,330, 5,174,993, 5,505,941, 5,338,683, 5,494,807, 4,722,848, 4,745,051, 4,879,236, 5,762,939, 5,858,368, 6,224,882, 6,103,526, 4,769,331, 5,591,439, 5,552,143, 5,591,639, 5,589,466, 5,580,859, 6,130,066, 6,004,777, 5,990,091, and 6,156,567. However, baculovirus vector systems and insect cells are presently preferred.

The expression product generated by vectors or recombinants in this invention are advantageously isolated and/or purified from infected or transfected cells or culture medium.

The DNA sequence coding for the BACE catalytic domain can be present in the vector operably linked to regulatory elements. In one embodiment of the present invention, insect host cells are preferably transfected with the recombinant hBACE_synth_his/pFastbac baculoviral DNA, thereby resulting in expression of the BACE catalytic domain. In another embodiment of the present invention, insect host cells are preferably transfected with the FURIN/pFastBac Dual baculoviral DNA, thereby resulting in expression of furin. Transfection and co-transfection with the recombinant molecules can be effected using methods well known in the art.

Host cells may be stably transfected or transiently transfected with a recombinant expression plasmid or infected by a recombinant virus vector. The host cells include prokaryotic cells, such as Escherichia coli, fungal systems such as Saccharomyces cerevisiae, permanent cell lines derived from insects such as Trichoplusia ni HighFive cells, Spodoptera frugiperda (SF-9) cells and Spodoptera frugiperda (SF-21) cells, Spodoptera frugiperda (SF900+, U.S. Pat. No. 6,103,066), and permanent mammalian cell lines such as Chinese hamster ovary (CHO) and SV40-transformed African green monkey kidney cells (COS).

The present invention contemplates “mutants” wherein a “mutant” refers to a polypeptide which is obtained by replacing at least one amino acid residue in a native or synthetic BACE catalytic domain with a different amino acid residue and/or by adding and/or deleting amino acid residues within the native polypeptide or at the N- and/or C-terminus of a polypeptide corresponding to a native BACE catalytic domain and which has substantially the same three-dimensional structure as the native BACE catalytic domain from which it is derived. Similarly, the present invention contemplates “mimics”; e.g., proteins that have substantially the same herein disclosed crystal structure of BACE. A mimic can be a mutant. By having substantially the same three-dimensional structure is meant having a set of atomic structure co-ordinates that have a root mean square deviation (r.m.s.d.) of less than or equal to about 2.0 Å when superimposed with the atomic structure co-ordinates of the native BACE catalytic domain from which the mutant is derived when at least about 50% to 100% of the C_{α} atoms of the native catalytic domain are included in the superposition. A mutant or mimic may have, but need not have, β-secretase activity.

The co-ordinates of Table 5 provide a measure of atomic location in Angstroms, to a third decimal place. The co-ordinates are a relative set of positions that define a shape in three dimensions, so it is possible that an entirely different set of co-ordinates and/or space group having a different origin and/or axes and/or space group could define a similar or identical shape. Furthermore, varying the relative atomic positions of the atoms of the structure so that the root mean square deviation of the residue backbone atoms (i.e., the nitrogen-carbon-carbon backbone atoms of the protein amino acid residues) is less than 1.5 Å (preferably less than 1.0 Å and more preferably less than 0.5 Å) when superimposed on the co-ordinates provided in Table 5 for the residue backbone atoms, will generally result in a structure which is substantially the same as the structure of Table 5 in terms of both its structural characteristics and potency for structure-based design or identification of BACE inhibitors or modulators. Likewise, changing the number and/or positions of the water molecules and/or substrate molecules of Table 5 will not generally affect the potency of the structure for structure-based design of BACE inhibitors or modulators. Thus, for the purposes described herein as being aspects of the present invention, it is within the scope of the present invention if: the Table 5 co-ordinates are transposed to a different origin and/or axes; the relative atomic positions of the atoms of the structures are varied so that the root mean square deviation of residue backbone atoms is less than 1.5 Å (preferably less than 1.0 Å and more preferably less than 0.5 Å) when superimposed on the co-ordinates provided in Table 5 for the residue backbone atoms; and/or the number and/or positions of water molecules and/or substrate molecules is varied. Reference herein to the data of Table 5 accordingly includes the co-ordinate data in which one or more individual values of the Table are varied in this way. By “root mean square deviation” is meant the square root of the arithmetic mean of the squares of the deviations from the mean.

As used herein, “Crystal or crystalline structure” or “crystalline form”: refers to a polypeptide in crystalline form. The term also includes co-crystals, as described herein. The term “co-crystal” refers to a crystal formed from a solution containing a mixture of the components i.e., polypeptide(s) and compound(s). Such compounds include, by way of example and not limitation, cofactors, substrates, substrate analogues, inhibitors, allosteric effectors, etc. Compounds include OM99-2, OM99-1 and a statine based peptide (Marcinkeviciene J., Luo Y., Gracian, N R., Combs Ap. And Copeland, R A. J. Biol Chem. 2001, 276:23790-23794). A soaked crystal is where a crystal is produced from one component (polypeptide) and then the other component is soaked in the compound(s).

The “binding” which is detected between a ligand and the active site, such as to determine inhibitors of BACE is an “association” between the ligand and the active site; and “association” refers to a condition of proximity between a chemical entity or compound, or portions or fragments thereof, and the BACE catalytic domain protein, or portions or fragments thereof. The association may be non-covalent, i.e., where the juxtaposition is energetically favored by, e.g., hydrogen-bonding, van der Waals, electrostatic or hydrophobic interactions, or it may be covalent.

The “active site” refers to that site in BACE domains where substrate peptide binding and cleavage occur. It is the site in BACE that is sought to be blocked by an inhibitor or ligand. A “functional portion” of a BACE protein includes at least the active site.

A “crystallographically-related dimer” is a dimer of two molecules wherein the symmetry axes or planes that relate the two molecules comprising the dimer coincide with the symmetry axes or planes of the crystal lattice, whereas a “non-crystallographically-related dimer” is a dimer of two molecules wherein the symmetry axes or planes that relate the two molecules comprising the dimer do not coincide with the symmetry axes or planes of the crystal lattice. And, “Bilobal structure:” refers to two globular lobes of the BACE protein and corresponds to the amino- and carboxy-terminal halves of the protein.

BACE contains a signal sequence, a pro-peptide, a catalytic aspartyl protease domain, a transmembrane region and a C-terminal cytoplasmic region. During transit through the endoplasmic reticulum, BACE undergoes constitutive N-terminal processing in the Golgi apparatus in which the pro-peptide is cleaved by a furin-like protease (Bennet et al 2000, Creemers et al 2001). More specifically, BACE undergoes a series of post-translational modifications including glycosylation, disulfide bond formation and propeptide processing. Haniu et al. have shown that BACE is N-glycosylated at four sites (Asn-153, Asn-172, Asn-223 and Asn-354) and that six Cys residues in the ectodomain form three intramolecular disulphide bonds (Cys216-Cys420, Cys278-Cys333 and Cys330-Cys380).

The present invention relates to crystalline polypeptides corresponding to the catalytic domain of BACE. Preferably, the crystalline catalytic domains are of sufficient quality to allow the determination of the three-dimensional X-ray diffraction structure to a resolution of better than, i.e., numerically lower than, 3.0 Å. The invention also relates to methods for preparing and crystallizing the polypeptides. The polypeptides themselves, as well as information derived from their crystal structures can be used to analyze and modify BACE as well as to identify compounds that interact with the catalytic domain. This can allow for the rational design or identification of compounds that inhibit or modulate BACE or interact with BACE or associate with BACE; which compounds have therapeutic value.

### Crystalline BACE

The crystals of the invention generally comprise substantially pure polypeptides corresponding to the BACE catalytic domain in crystalline form.

It is to be understood that the crystalline BACE catalytic domains of the invention are not limited to synthetic BACE domains. Indeed, the crystals of the invention also include native BACE catalytic domains and mutants and mimics of the BACE catalytic domain.

Amino acid substitutions, deletions and additions which do not significantly interfere with the three-dimensional structure of the BACE domain will depend, in part, on the region of the BACE domain where the substitution, addition or deletion occurs. In highly variable regions of the molecule, non-conservative substitutions as well as conservative substitutions may be tolerated without significantly disrupting the three-dimensional structure of the molecule. In highly conserved regions, or regions containing significant secondary structure, conservative amino acid substitutions are preferred.

Conservative amino acid substitutions are well-known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

In some instances, it may be particularly advantageous or convenient to substitute, delete and/or add amino acid residues to a BACE catalytic domain in order to provide convenient cloning sites in cDNA encoding the polypeptide, to aid in purification of the polypeptide, etc. Such substitutions, deletions and/or additions which do not substantially alter the three dimensional structure of the BACE catalytic domain will be apparent to those having skills in the art.

It should be noted that the mutants contemplated herein need not exhibit enzymatic activity. Indeed, amino acid substitutions, additions or deletions that interfere with the β-secretase activity of the BACE domain but which do not significantly alter the three-dimensional structure of the domain are specifically contemplated by the invention. Such crystalline polypeptides, or the atomic structure co-ordinates obtained therefrom, can be used to identify compounds that bind to the native domain.

The co-crystals of the invention generally comprise a crystalline BACE domain polypeptide in association with one or more compounds. The association may be covalent or non-covalent. Such compounds include, but are not limited to, cofactors, substrates, substrate analogues, inhibitors, allosteric effectors, etc.

### Production of Polypeptides

The synthetic and mutated BACE catalytic domain polypeptides described herein may be chemically synthesized in whole or part using techniques that are well-known in the art (see, e.g.; Kochendoerfer G G (2001) “Chemical protein synthesis methods in drug discovery”. Current Opinion in Drug Discovery and Development 4, 205-214). Alternatively, methods which are well known to those skilled in the art can be used to construct expression vectors containing the synthetic or mutated BACE domain polypeptide coding sequence and appropriate transcriptional/translational control signals. These methods include in vitro recombinant DNA techniques, synthetic techniques and in vivo recombination/genetic recombination. See, for example, the techniques described in Maniatis et al., 1989.

A variety of host-expression vector systems may be utilized to express the synthetic BACE domain coding sequence. These include but are not limited to insect cell systems infected with recombinant virus (e.g., baculovirus) containing the BACE domain coding sequence or animal cell systems; microorganisms such as bacteria transformed with recombinant bacteriophage DNA, plasmid DNA or cosmid DNA expression vectors containing the BACE domain coding sequence and yeast transformed with recombinant yeast expression vectors containing the BACE domain coding sequence. The expression elements of these systems vary in their strength and specificities. Depending on the host/vector system utilized, any of a number of suitable transcription and translation elements, including constitutive and inducible promoters, may be used in the expression vector. For example, when cloning in insect cell systems, promoters such as the baculovirus polyhedrin promoter may be used; in bacterial systems, inducible promoters such as pL of bacteriophage .mu., plac, ptrp, ptac (ptrp-lac hybrid promoter) and the like may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used and when generating cell lines that contain multiple copies of the BACE catalytic domain DNA, SV40-, BPV- and EBV-based vectors may be used with an appropriate selectable marker.

### Crystallization of Polypeptides and Characterization of Crystal Structure

The crystals of the invention can be obtained by conventional means as are well-known in the art of protein crystallography, including batch, liquid bridge, dialysis, vapor diffusion and hanging drop methods (see, e.g., McPherson, 1982; McPherson, 1990; Webber, 1991).

Generally, the crystals of the invention are grown by dissolving substantially pure synthetic BACE domain polypeptide in an aqueous buffer containing a precipitant at a concentration just below that necessary to precipitate the protein. Water is removed by controlled evaporation to produce precipitating conditions, which are maintained until crystal growth ceases.

In a preferred embodiment of the invention, native crystals are grown by vapor diffusion in hanging drops (McPherson, 1982 and 1990). In this method, the polypeptide/precipitant solution is allowed to equilibrate in a closed container with a larger aqueous reservoir having a precipitant concentration optimal for producing crystals. Generally, equal volumes of a substantially pure polypeptide solution are mixed with an equal volume of reservoir solution, giving a precipitant concentration about half that required for crystallization. This solution is suspended as a droplet underneath a coverslip, which is sealed onto the top of the reservoir. The sealed container is allowed to stand, usually for about 2-6 weeks, until crystals grow.

Thus, the invention provides a method for crystallizing BACE which comprises producing a BACE protein, e.g., by recombinant production via a suitable host and/or vector such as through expression in insect cells, recovering the BACE and growing crystals from the recovered BACE. The BACE so produced is suitable for X-ray diffraction analysis. And, the growing of the crystals can be by any suitable means, advantageously the hanging drop method.

### Uses of the Crystals and Atomic Structure Co-Ordinates

The crystals of the invention, and particularly the atomic structure co-ordinates obtained therefrom, have a wide variety of uses. The crystals (either apo or co-complexed) and structure co-ordinates (either apo or co-complexed) are particularly useful for identifying compounds that inhibit β-secretase activity as an approach towards developing new therapeutic agents.

The structure co-ordinates described herein can be used as phasing models in determining the crystal structures of additional synthetic or mutated BACE domains, as well as the structures of co-crystals of such domains with ligands such as inhibitors, agonists, antagonists, etc. The structure co-ordinates, as well as models of the three-dimensional structures obtained therefrom, can also be used to aid the elucidation of solution-based structures of synthetic or mutated BACE domains, such as those obtained via nuclear magnetic resonance (NMR).

The provision of the structure of BACE crystals in Table 5 provides the skilled artisan with a detailed insight into the mechanisms of action of BACE. This insight provides a means to design inhibitors of BACE which can be used for inhibiting BACE or the production of Aβ or fragments thereof or treating AD or disorders involving the production of Aβ or fragments thereof (which disorders are treatable by inhibition of BACE) in an individual in need thereof.

The provision of the crystal structure of BACE allows a novel approach for drug discovery, identification, and design for modulators, e.g., inhibitors, of BACE. Accordingly, the invention provides a computer-based method of rational drug design or identification which comprises: providing the structure of BACE as defined by the co-ordinates or the identifying co-ordinates in Table 5; providing a structure of a candidate modulator or inhibitor; and fitting the structure of the candidate to the structure of BACE of Table 5.

In an alternative aspect, the method may use the co-ordinates of atoms of interest of BACE which are in the vicinity of the active site or binding region in order to model the pocket in which the substrate or ligand binds. These co-ordinates may be used to define a space which is then screened “in silico” against a candidate modulator molecule. Thus, the invention provides a computer-based method of rational drug design or identification which comprises: providing the co-ordinates of at least two atoms of Table 5 of BACE (“selected co-ordinates”); providing the structure of a candidate modulator or inhibitor; and fitting the structure of the candidate to the selected co-ordinates of BACE.

In practice, it may be desirable to model a sufficient number of atoms of BACE as defined by the co-ordinates of Table 5 which represent the active site or binding region. Thus, there can be provided the co-ordinates of at least 5, advantageously at least 10, more advantageously at least 50 and even more advantageously at least 100 atoms of the BACE structure.

Accordingly, the methods of the invention can employ a sub-domain of interest of BACE which is in the vicinity of the active site or binding region, and the invention can provide a computer-based method for identifying or rationally designing a drug which comprises: providing the co-ordinates of at least a sub-domain of BACE; providing the structure of a candidate modulator or inhibitor of BACE; and fitting the structure of the candidate to the co-ordinates of the BACE sub-domain provided.

These methods can optionally include synthesizing the candidate and can optionally further include contacting the candidate with BACE to test whether there is binding and/or inhibition.

“Fitting” can mean determining, by automatic or semi-automatic means, interactions between at least one atom of the candidate and at least one atom of BACE and calculating the extent to which such an interaction is stable. Interactions can include attraction, repulsion, brought about by charge, steric considerations, and the like. A “sub-domain” can mean at least one, e.g., one, two, three, or four, complete element(s) of secondary structure. Particular regions of BACE include those identified in Table 5.

Modulators of BACE may be inhibitors of BACE or compounds which affect its specificity or activity in other ways. Advantageously, modulators are inhibitors.

The step of providing the structure of a candidate modulator molecule may involve selecting the compound by computationally screening a database of compounds for interaction with the active site. For example, a 3-D descriptor for the potential modulator may be derived, the descriptor including geometric and functional constraints derived from the architecture and chemical nature of the active site. The descriptor may then be used to interrogate the compound database, a potential modulator being a compound that has a good match to the features of the descriptor. In effect, the descriptor can be a type of virtual pharmacophore.

In any event, the determination of the three-dimensional structure of BACE provides a basis for the design of new and specific modulators for BACE. For example, from knowing the three-dimensional structure of BACE, computer modelling programs may be used to design or identify different molecules expected to interact with possible or confirmed active sites such as binding sites or other structural or functional features of BACE.

More specifically, a potential modulator of BACE activity can be examined through the use of computer modeling using a docking program such as GRAM, DOCK or AUTODOCK (see Walters et al. Drug Discovery Today, vol. 3, no. 4 (1998), 160-178, and Dunbrack et al. Folding and Design 2 (1997), 27-42) to identify potential inhibitors of BACE. This procedure can include computer fitting of potential modulators to BACE to ascertain how well the shape and the chemical structure of the potential modulator (e.g., inhibitor) will bind to the enzyme.

Also, computer-assisted, manual examination of the active site or binding site of BACE may be performed. The use of programs such as GRID (P. Goodford, J. Med. Chem, 1985, 28, 849-57)—program that determines probable interaction sites between molecules with various functional groups and the enzyme surface—may also be used to analyze the active site or binding site to predict partial structures of modulating compounds.

Computer programs can be employed to estimate the attraction, repulsion or steric hindrance of the two binding partners, e.g., BACE and a candidate inhibitor. Generally, the tighter the fit, the fewer the steric hindrances, and the greater the attractive forces, the more potent the potential modulator, since these properties are consistent with a tighter binding constant. Furthermore, the more specificity in the design of a candidate modulator, the more likely it is that it will not interact with other proteins as well. This will tend to minimize potential side-effects due to unwanted interactions with other proteins.

In a further aspect the invention provides for a method for determining the structure of a modulator of BACE bound to BACE, said method comprising, (a) providing a crystal of BACE according to the invention, (b) soaking the crystal with said modulator; and (c) determining the structure of said BACE-modulator complex.

The invention further involves, in place of or in addition to in silico methods, high throughput screening of compounds to select compounds with binding activity. Those compounds which show binding activity may be selected as possible candidate modulators, and further crystallized with BACE, e.g., by co-crystallization or by soaking, for X-ray analysis. The resulting X-ray structure may be compared with that of Table 5 for a variety of purposes. For example, where the contacts made by such compounds overlap with those made by BACE, novel molecules comprising residues which contain contacts of BACE and other compounds may be provided.

Having designed, identified, or selected possible binding candidate modulators or inhibitors by determining those which have favorable fitting properties, e.g., strong attraction between a candidate and BACE, these can be then screened for activity. Consequently, the invention further involves: obtaining or synthesizing the candidate modulator or inhibitor; and contacting the candidate modulator or inhibitor with BACE to determine the ability of the candidate to inhibit or modulate or interact with BACE. In the latter step, the candidate is advantageously contacted with BACE under conditions to determine its function. Instead of, or in addition to, performing such an assay, the invention may comprise: obtaining or synthesizing the candidate modulator, forming a complex of BACE and the candidate, and analyzing the complex, e.g., by X-ray diffraction or NMR or other means, to determine the ability of the candidate to interact with BACE. Detailed structural information can then be obtained about the binding of the candidate to BACE, and in light of this information, adjustments can be made to the structure or functionality of the potential modulator, e.g., to improve its binding to BACE. These steps may be repeated and re-repeated as necessary. Advantageously, in the contacting step, the potential modulator is contacted with BACE in the presence of a substrate and typically a buffer, to determine the ability of the potential modulator to alter the function of BACE.

The invention further involves a method of determining three dimensional structures of BACE homologues of unknown structure by using the structural co-ordinates of Table 5. For example, if X-ray crystallographic or NMR spectroscopic data is provided for a BACE homologue of unknown structure, the structure of BACE as defined in Table 5 may be used to interpret that data to provide a likely structure for the BACE homologue by techniques well known in the art, e.g., by phase modeling in the case of X-ray crystallography. Thus, an inventive method can comprise: aligning a representation of an amino acid sequence of a BACE homologue of unknown structure with the amino acid sequence of BACE to match homologous regions of the amino acid sequences; modeling the structure of the matched homologous regions of the BACE of unknown structure on the structure as defined in Table 5 of the corresponding regions of BACE; and, determining a conformation (e.g. so that favorable interactions are formed within the BACE of unknown structure and/or so that a low energy conformation is formed) for the BACE of unknown structure which substantially preserves the structure of said matched homologous regions. “Homologous regions” describes amino acid residues in two sequences that are identical or have similar, e.g., aliphatic, aromatic, polar, negatively charged, or positively charged, side-chain chemical groups. Identical and similar residues in homologous regions are sometimes described as being respectively “invariant” and “conserved” by those skilled in the art. Advantageously, the first and third steps are performed by computer modeling. Homology modeling is a technique that is well known to those skilled in the art (see, e.g., Greer, Science vol. 228 (1985) 1055, and Blundell et al. Eur J Biochem vol 172 (1988), 513).

In general, comparison of amino acid sequences is accomplished by aligning an amino acid sequence of a polypeptide of a known structure with the amino acid sequence of a the polypeptide of unknown structure. Amino acids in the sequences are then compared and groups of amino acids that are homologous are grouped together. This method detects conserved regions of the polypeptides and accounts for amino acid insertions and deletions. Homology between amino acid sequences can be determined by using commercially available algorithms. In addition to those otherwise mentioned herein, mention is made too of the programs BLAST, gapped BLAST, BLASTN, and PSI-BLAST, provided by the National Center for Biotechnology Information. These programs are widely used in the art for this purpose and can align homologous regions of two amino acid sequences.

Once the amino acid sequence of the polypeptides with known and unknown structures are aligned, the structures of the conserved amino acids in a computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure. The structures of amino acids located in non-conserved regions may be assigned manually using standard peptide geometries or by molecular simulation techniques, such as molecular dynamics. Refining the entire structure can be by molecular dynamics and/or energy minimization.

The aspects of the invention which employ the BACE structure in silico may be equally applied to homologue models of BACE obtained by the above aspect of the invention and this forms yet a further embodiment of the invention. Thus, having determined a conformation of a BACE by the methods described herein, such a conformation may be used in a computer-based method of rational drug or compound design or identification as described herein.

The invention further provides a method for determining the structure of a modulator of BACE bound to BACE comprising: providing a crystal of BACE, e.g., according to the invention, soaking the crystal with the modulator, and determining the structure of the BACE-modulator complex. Alternatively or additionally the BACE and the modulator may be co-crystallized.

Having obtained and characterized a modulator according to the invention, the invention further provides a method for modulating the activity of BACE which comprises: providing BACE under conditions where, in the absence of a modulator, BACE is able to exhibit secretase activity, providing a modulator compound (e.g., contacting the modulator and the BACE), determining the extent to which the activity of BACE is altered by the presence of the modulator compound.

The invention further provides systems, such as computer systems, intended to generate structures and/or perform rational drug design for a BACE or complex of BACE and a potential modulator. The system can contain: atomic co-ordinate data according to Table 5 or derived therefrom by homology modeling, said data defining the three-dimensional structure of a BACE or at least one sub-domain thereof; or structure factor data for BACE, said structure factor data being derivable from the atomic co-ordinate data of Table 5. The invention also involves computer readable media with: atomic co-ordinate data according to Table 5 or derived therefrom by homology modeling, said data defining the three-dimensional structure of a BACE or at least one sub-domain thereof; or structure factor data for BACE, said structure factor data being derivable from the atomic co-ordinate data of Table 5. “Computer readable media” refers to any media which can be read and accessed directly by a computer, and includes, but is not limited to: magnetic storage media such as floppy discs, hard storage medium and magnetic tape; optical storage media such as optical discs or CD-ROM; electrical storage media such as RAM and ROM; and hybrids of these categories, such as magnetic/optical media. By providing such computer readable media, the atomic co-ordinate data can be routinely accessed to model BACE or a sub-domain thereof. For example RASMOL (Sayle et al., TIBS vol. 20 (1995), 374) is a publicly available software package which allows access and analysis of atomic co-ordinate data for structural determination and/or rational drug design. The invention further comprehends methods of doing business by providing access to such computer readable media and/or computer systems and/or atomic co-ordinate data to users; e.g., the media and/or atomic co-ordinate data can be accessible to a user, for instance on a subscription basis, via the Internet or a global communication/computer network; or, the computer system can be available to a user, on a subscription basis. Structure factor data, which are derivable from atomic co-ordinate data (see, e.g., Blundell et al., in Protein Crystallography, Academic Press, NY, London and San Francisco (1976)), are particularly useful for calculating, e.g., difference Fourier electron density maps. Thus, there are additional uses for the computer readable media and/or computer systems and/or atomic co-ordinate data and additional reasons to provide them to users. A “computer system” refers to the hardware means, software means and data storage means used to analyze the atomic co-ordinate data of the present invention. The minimum hardware means of computer-based systems of the invention may comprise a central processing unit (CPU), input means, output means, and data storage means. Desirably, a monitor is provided to visualize structure data. The data storage means may be RAM or other means for accessing computer readable media of the invention. Examples of such systems are microcomputer workstations available from Silicon Graphics Incorporated and Sun Microsystems running Unix based, Windows NT or IBM OS/2 operating systems.

The invention also provides a method of analyzing a complex of BACE and a potential modulator comprising: employing X-ray crystallographic diffraction data from the complex and a three-dimensional structure of BACE or at least a sub-domain thereof, to generate a difference Fourier electron density map of the complex; advantageously, the three-dimensional structure being as defined by the atomic co-ordinate data according to Table 5.

Such complexes can be crystallized and analyzed using X-ray diffraction methods, e.g., according to the approaches described by Greer et al., J of Medicinal Chemistry, vol 37 (1994), 1035-54, and difference Fourier electron density maps can be calculated based on X-ray diffraction patterns of soaked or co-crystallized BACE and the solved structure of uncomplexed BACE. These maps can then be used to determine whether and where a particular potential modulator binds to BACE and/or changes the conformation of BACE. Electron density maps can be calculated using programs such as those from the CCP4 computer package (Collaborative Computing Project, No. 4. The CCP4 Suite: Programs for Protein Crystallography, Acta Crystallographica, D50, 1994, 760-763). For map visualization and model building programs such as “QUANTA” (1994, San Diego, Calif.: Molecular Simulations, Jones et al., Acta Crystallography A47 (1991), 110-119) can be used.

Table 5 gives atomic co-ordinate data for BACE complexed with OM99-2, and lists each atom by a unique number; the chemical element and its position in each amino acid residue, the amino acid residue in which the element is located, the chain identifier, the number of the residue, co-ordinates (e.g., X, Y, Z) which define with respect to the crystallographic axes the atomic position (in A) of the respective atom, the occupancy of the atom in the respective position, “B”, isotropic displacement parameter (in A²) which accounts for movement of the atom around its atomic center, and atomic number.

Determination of the 3D structure of BACE provides important information about the likely active site(s) of BACE, particularly when comparisons are made with other enzymes, such as similar enzymes. This information may be used for rational design of BACE inhibitors, e.g., by computational techniques that identify possible binding ligands for the active site(s), by enabling linked-fragment approaches to drug design, and by enabling the identification and location of bound ligands using analyses such as X-ray crystallographic analysis.

Greer et al., supra, relates to an iterative approach to ligand design based on repeated sequences of computer modeling, protein-ligand complex formation, and X-ray analysis. Thymidylate synthase inhibitors were designed by Greer; and, BACE inhibitors may also be designed in this way. Using, for example, GRID (P. Goodford, J. Med. Chem, 1985, 28, 849-57) or the solved 3D structure of BACE, a potential modulator of BACE may be designed that complements the functionalities of the BACE active site(s). The potential modulator can be synthesized, formed into a complex with BACE, and the complex then analyzed, e.g., by X-ray crystallography, NMR or a combination thereof, to identify the actual position of the bound compound.

Determination of the position of the potential modulator compound in the complex allows determination of the interactions of it with BACE. This allows the skilled artisan to analyze the affinity and specificity of the compound for BACE, and to propose modifications to the compound to increase or decrease either or both of these properties. Thus, the structure and/or functional groups of the compound can then be adjusted, if necessary or desired, in view of the results from the analysis (e.g., X-ray analysis), and the synthesis and analysis sequence repeated until an optimized compound is obtained. Related approaches to structure-based drug design are also discussed in other documents cited herein, as well as in Bohacek et al., Medicinal Research Reviews, vol. 16 (1996), 3-5.

As a result of the determination of the BACE 3D structure, more purely computational techniques for rational drug design may also be used to design BACE modulators; for example, automated ligand-receptor docking programs (see Jones et al., in Current Opinion in Biotechnology, vol 6 (1995), 652-656) which require accurate information on the atomic co-ordinates of target receptors, may be used to design or identify potential BACE modulators or inhibitors.

Linked-fragment approaches to drug design also require accurate information on the atomic co-ordinates of a target. Small compounds that have the potential to bind to regions of BACE which in themselves may not be modulator compounds may be assembled by chemical linkage to provide potential modulators. Thus, the basic idea behind these approaches is to determine the binding locations of more than one, e.g., plural or a plurality of, ligands to a target molecule, and then construct a molecular scaffold to connect the ligands together in such a way that their relative binding positions are preserved. The ligands may be provided computationally and modeled in a computer system, or provided in an experimental setting, wherein crystals according to the invention are provided and more than one, e.g., plural or a plurality of, ligands soaked separately or in mixed pools into the crystal prior to analysis, e.g., X-ray analysis, and determination of their location.

The binding site of two or more ligands are determined and may be connected to thus form a potential lead compound that can be further refined, e.g., the iterative technique of Greer et al. For a virtual linked-fragment approach, see Verlinde et al., J of Computer-Aided Molecular Design 6 (1992), 131-147 and for NMR and X-ray approaches, see Skuker et al., Science 274 (1996), 1531-1534, and Stout et al., Structure 6 (1998), 839-48. The use of these or other approaches to design and/or identify BACE modulators (see, e.g., patent documents cited herein such as in the Background Section, supra) is made possible by the determination of the BACE structure.

Many of the techniques and approaches to structure-based described herein employ X-ray analysis to identify the binding position of a potential modulator in a complex with a protein. A common way of doing this is to perform X-ray crystallography on the complex, produce a difference Fourier electron density map, and associate a particular pattern of electron density with the potential modulator. However, to produce a map (See Blundell et al., supra), it is important to know the 3D structure of the protein beforehand (or at least the protein structure factors). Therefore, determination of the BACE structure also allows difference Fourier electron density maps of complexes of BACE with a potential modulator to be produced, which can greatly assist in the process of rational compound and/or drug design or identification.

The approaches to structure-based drug or compound design or identification described herein involve initial identification of possible compounds for interaction with the target molecule (in this case BACE). Sometimes these compounds are known, e.g., from research literature. However, when they are not, or when novel compounds are wanted, a first stage of the drug or compound design or identification program may involve computer-based in silico screening of compound databases (such as the Cambridge Structural Database) with the aim of identifying compounds which interact with the active site or sites of the target bio-molecule (in this case BACE). Screening selection criteria may be based on pharmacokinetic properties such as metabolic stability and toxicity. However, determination of the BACE structure allows the architecture and chemical nature of each BACE active site to be identified, which in turn allows the geometric and functional constraints of a descriptor for the potential inhibitor to be derived. The descriptor can be, therefore, a type of virtual 3D pharmacophore, which can also be used as selection criteria or filter for database screening.

Compounds which have a chemical structure selected using the invention, wherein said compounds are BACE modulators or inhibitors, form a further aspect of the invention; and, such compounds may be used in methods of medical treatments, such as for inhibiting BACE or the production of Aβ or fragments thereof or treating AD or other maladies involving BACE or the production of Aβ or fragments thereof. Further, such compounds may be used in the preparation of medicaments for such treatments. The compounds may be employed alone or in combination with other treatments for inhibiting BACE or the production of Aβ or fragments thereof or treating AD or other maladies involving BACE or the production of Aβ or fragments thereof; and, the compounds may be used in the preparation of combination medicaments for such treatments, or in kits containing the compound and the other treatment.

Turning more specifically to BACE, BACE is a pepsin-like aspartyl proteinase, the mature enzyme consisting of the N-terminal catalytic domain, a transmembrane domain, and a small cytoplasmic domain. BACE has an optimum activity at pH 4.5 (Vassar et al, 1999) or pH 5.0 (Yan et al. 1999) and is found in acidic subcellular compartments such as golgi and endosomes (Vassar et al., 1999 and Capell et al., 2000). The pH in the endosome and trans golgi network, where BACE appears to function, fluctuates in the range of pH 4.5-6.0 with the average pH being stated as 5.0 (Lee et al. 1996) and pH 5.4 (Overly et al. 1995). BACE is not inhibited by standard pepsin inhibitors such as pepstatin. It has been shown that the catalytic domain minus the transmembrane and cytoplasmic domain has activity against substrate peptides (Lin et al, 2000). Consequently, this soluble catalytic domain is suitable for crystallization studies and a crystal structure of this will give a representative structure of the BACE active site for the design of inhibitor molecules. Ideally it would be desirable to crystallize a form of BACE with an unoccupied active site. This could be used to soak in small molecule inhibitors of the enzyme and to investigate their binding modes. Crystals of BACE grown both in the presence and absence of inhibitor, having same space group and similar unit cell parameters are described. These crystals are grown between pH 5.6 and pH 5.8 and thus are grown at the biologically relevant pH of BACE. This is also close to the optimum pH of the enzyme. Upon soaking the C2 crystal form with a ligand, some reorganization of the molecules in the crystal will take place, resulting in a space group change from C2 to P2₁. The cell dimensions and the packing of the P2 form are closely related to those of the C2 form. Because the BACE crystals are grown at physiologically relevant pH the compounds identified in accordance with the invention would be of more biological relevance. The lead compounds/inhibitors generated may be of higher therapeutic value and would truly reflect the mode of inhibition in vivo, particularly for those compounds that are susceptible to changes in protonation state.

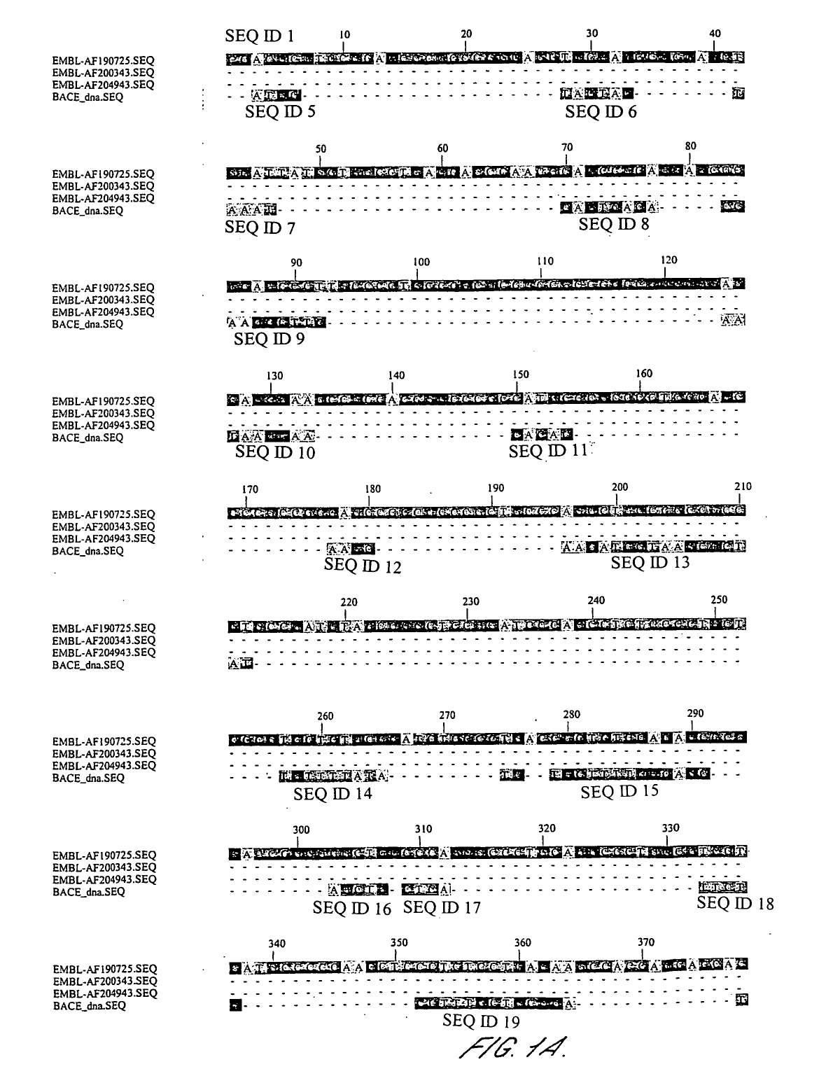

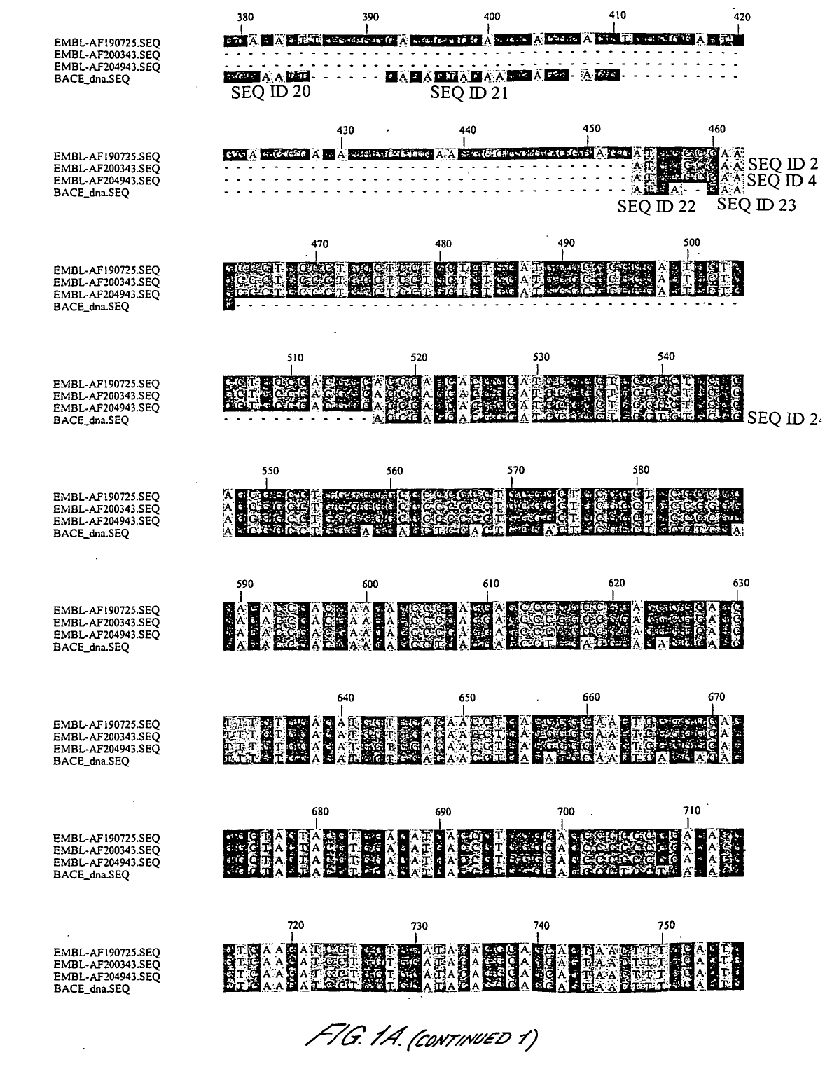

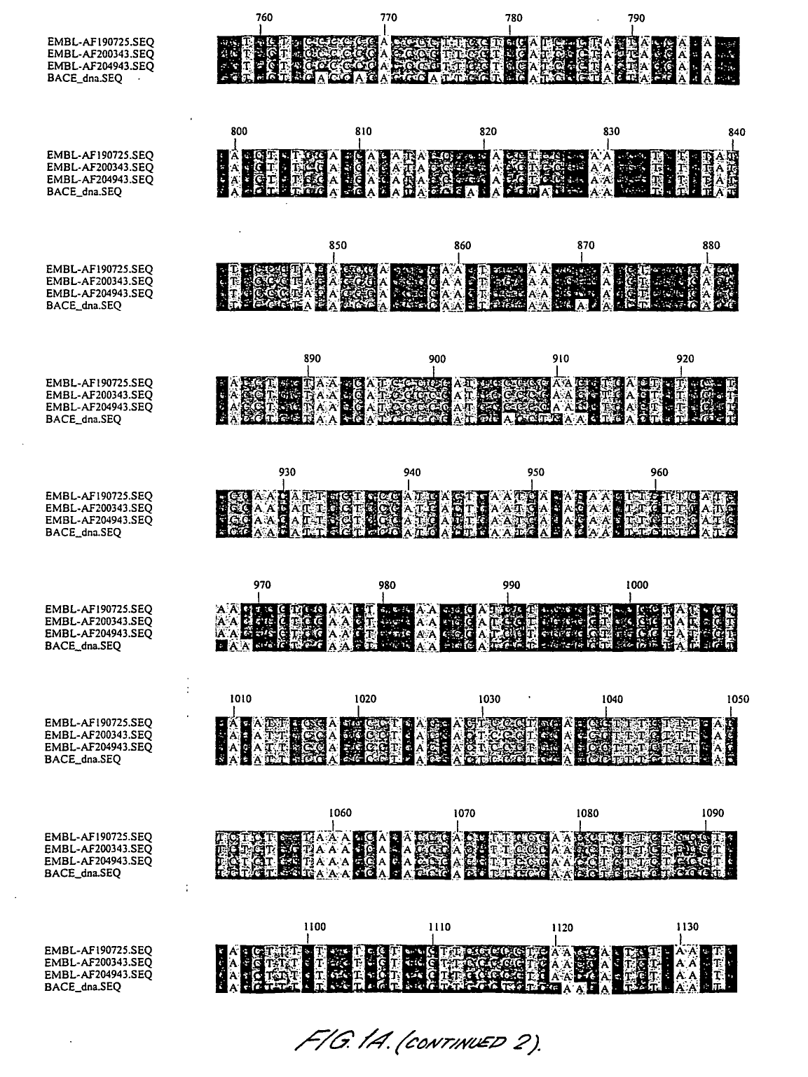

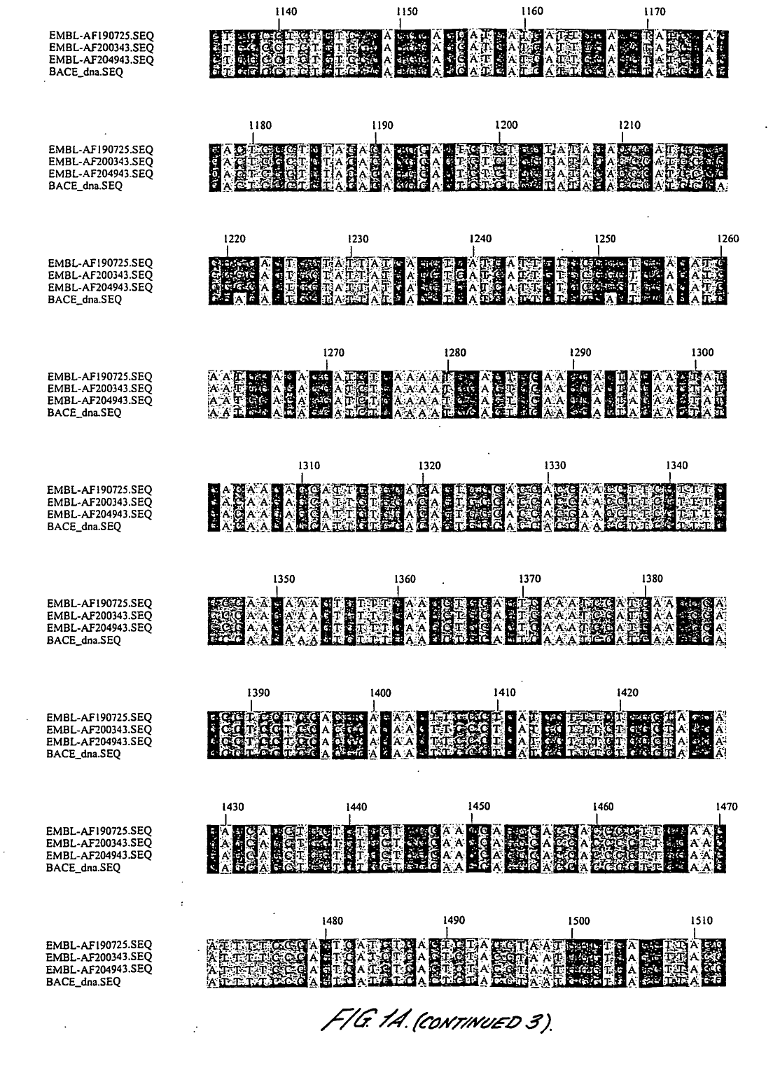

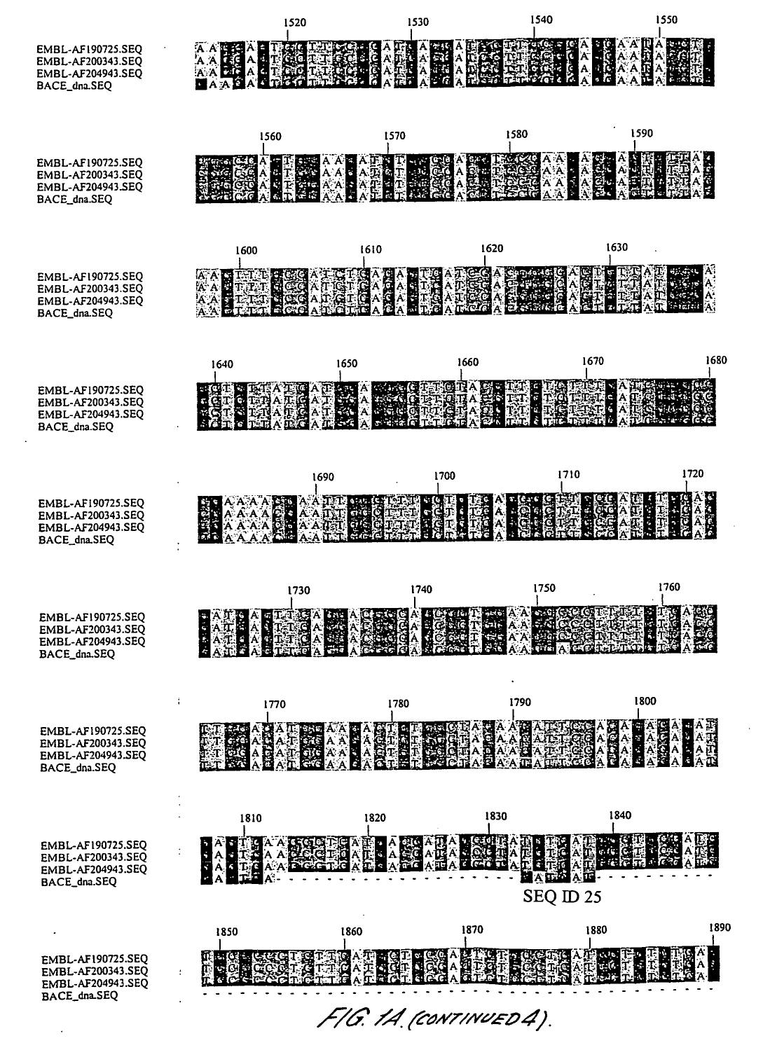

A synthetic gene encoding the pro- and aspartyl protease domains of BACE was constructed (see Example 1). The construct extended from Thr 22 to Ser 453 (numbering refers to the full-length BACE sequence, e.g. Genbank accession P56817, SEQ ID NO:6). In each of the four potential glycosylation sites (Asn-X-Ser/Thr: Asparagines-153, -172, -223 and -354) the Asparagine residue was mutated to Glutamine to prevent glycosylation of the protein. Silent mutations were also introduced into the coding sequence in order to reduce the GC content of the gene (FIG. 1A shows an alignment of the synthetic DNA sequence of the present invention with other wild-type BACE genes). A His₆ peptide tag was added to the C-terminus of the protein sequence to facilitate purification on Nickel agarose (see Example 1).

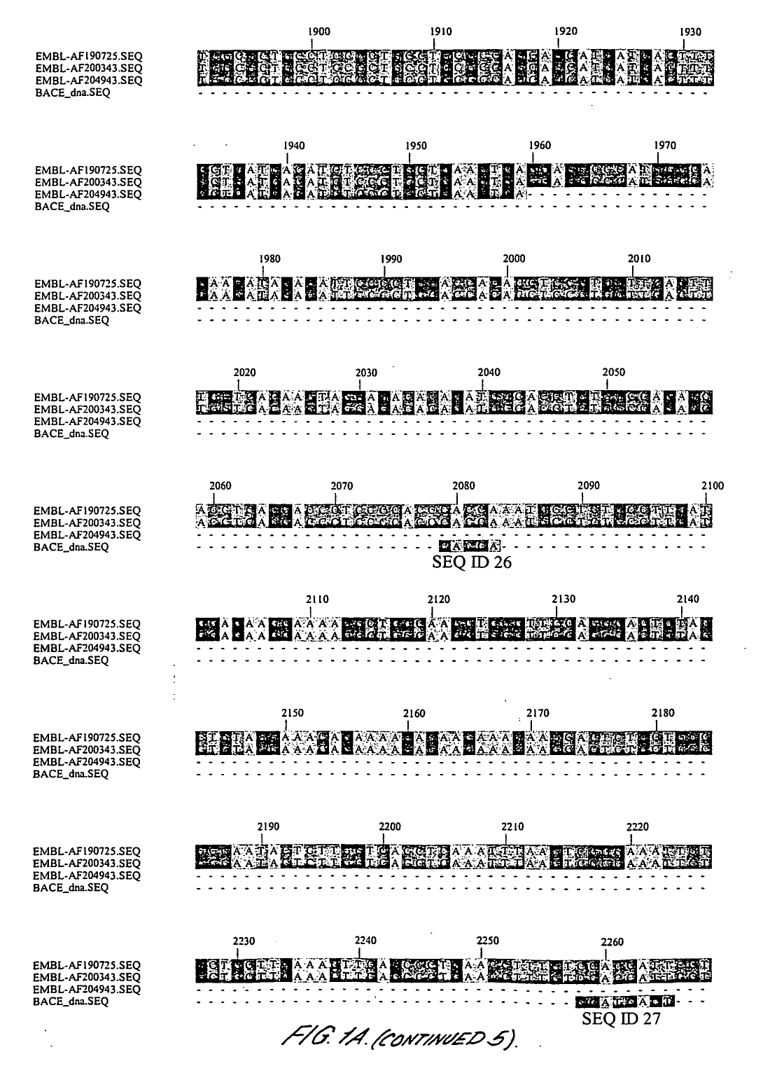

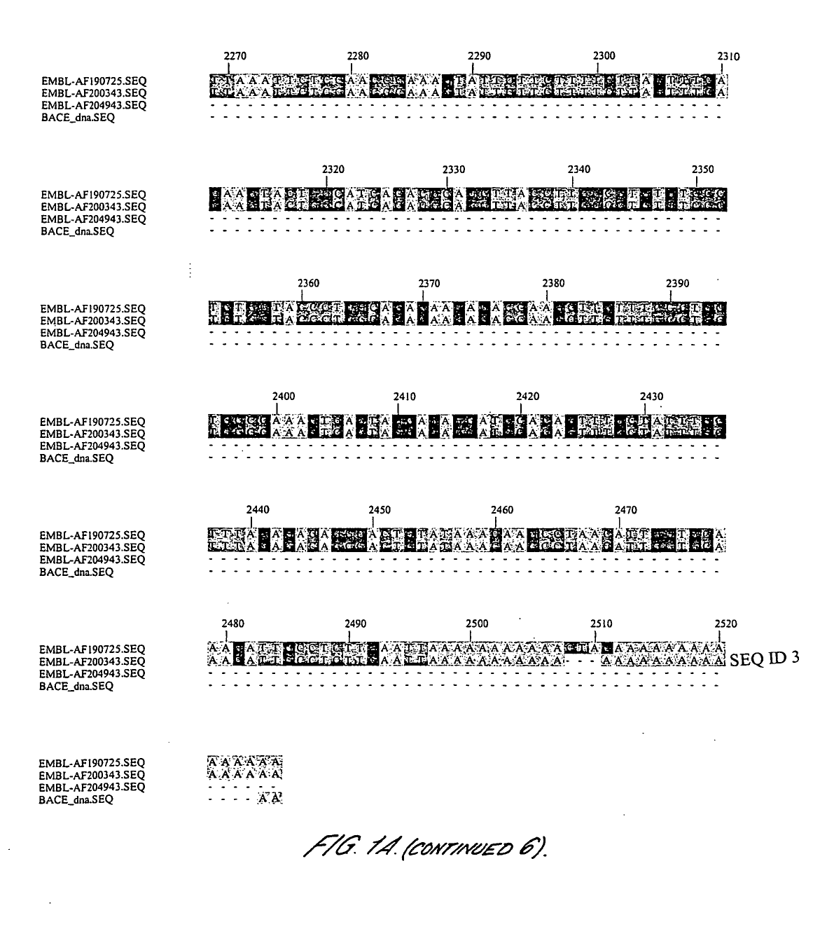

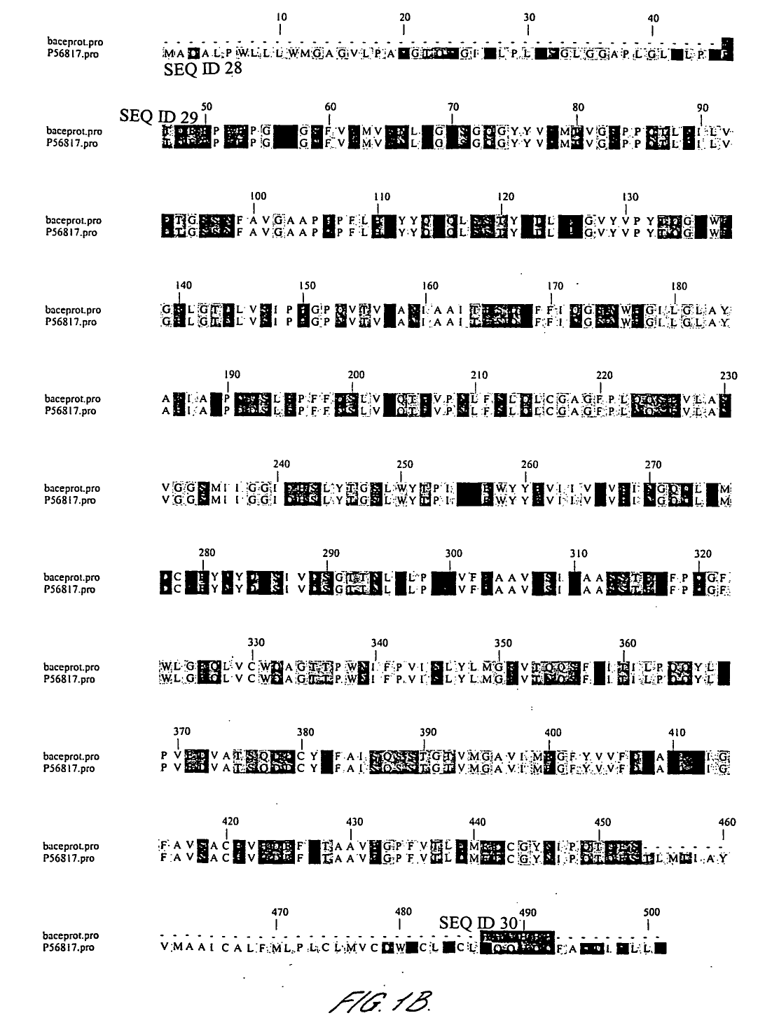

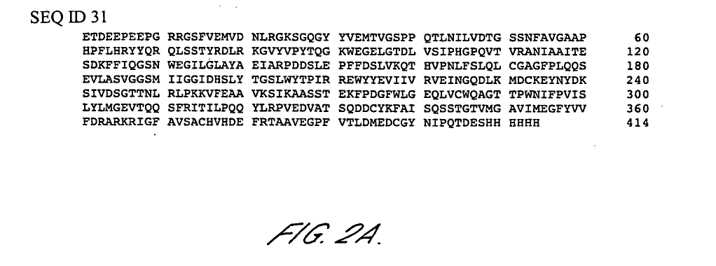

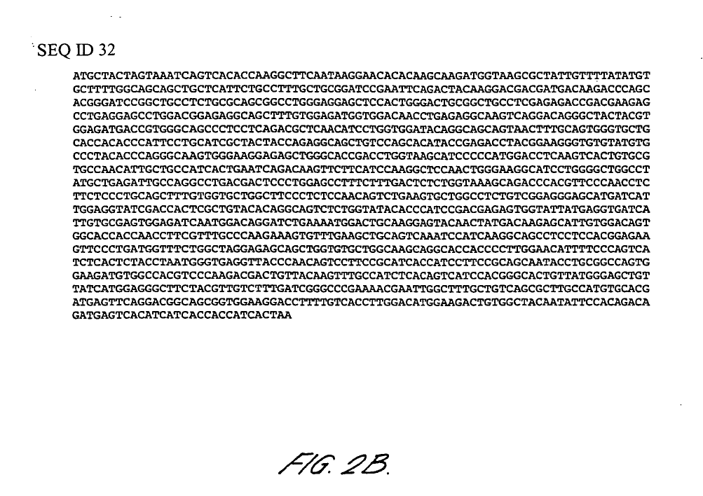

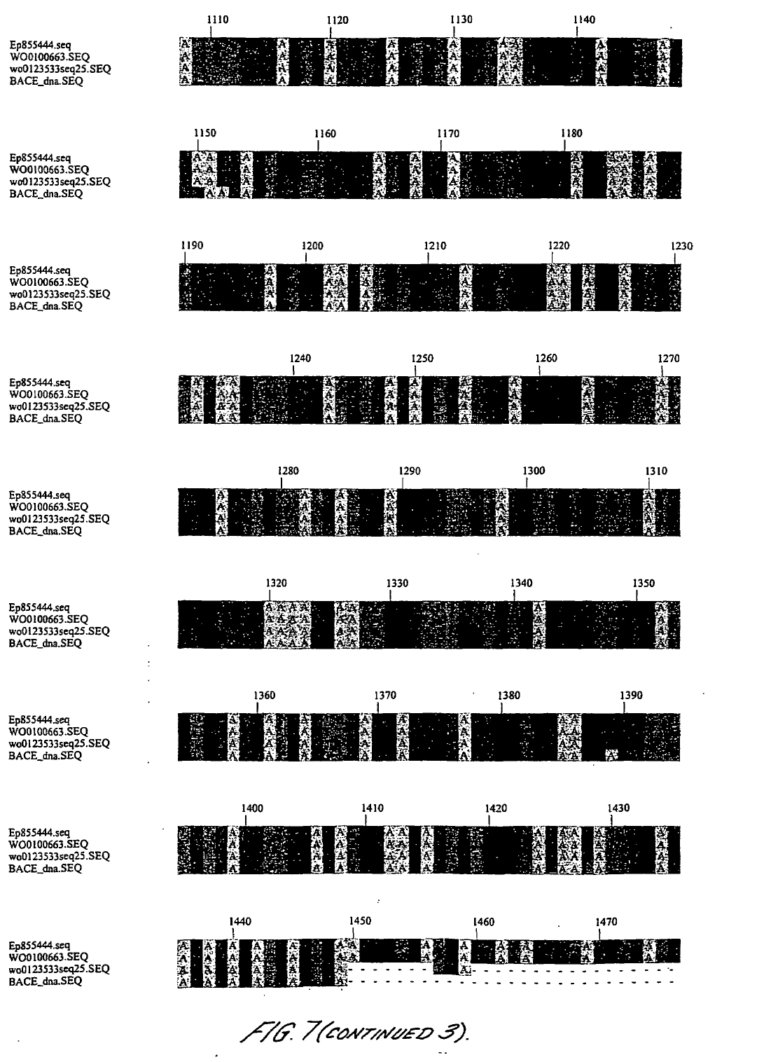

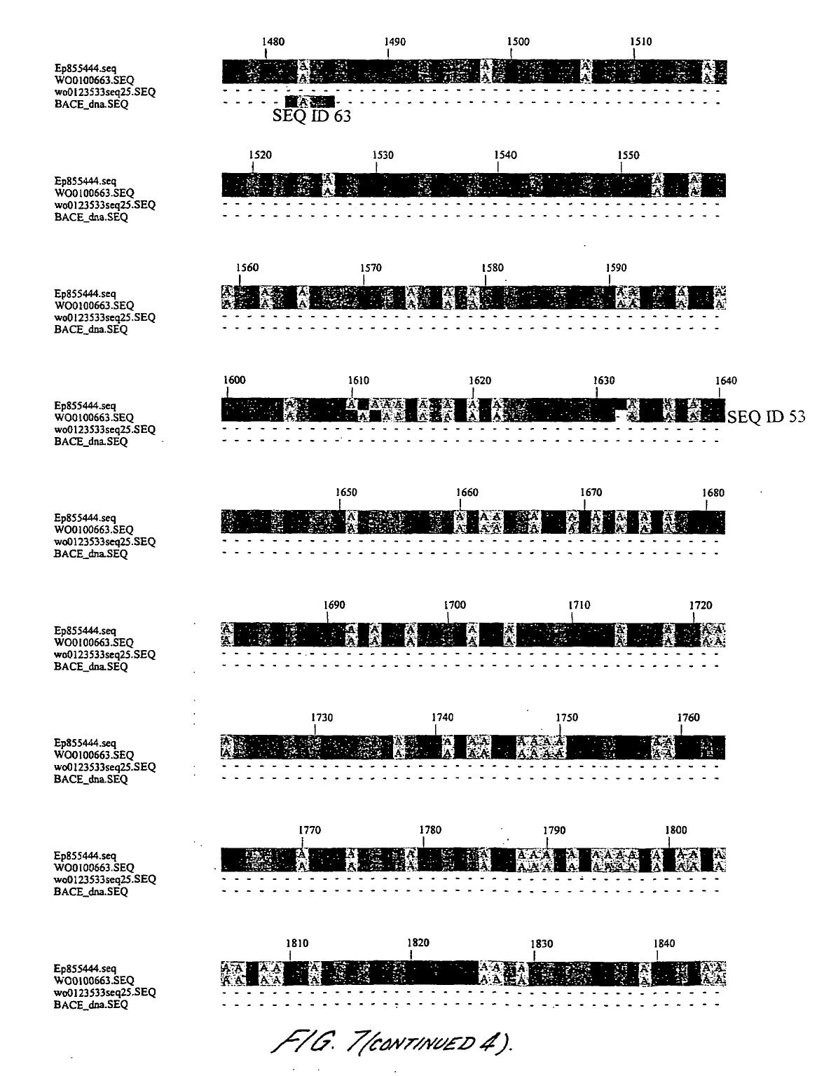

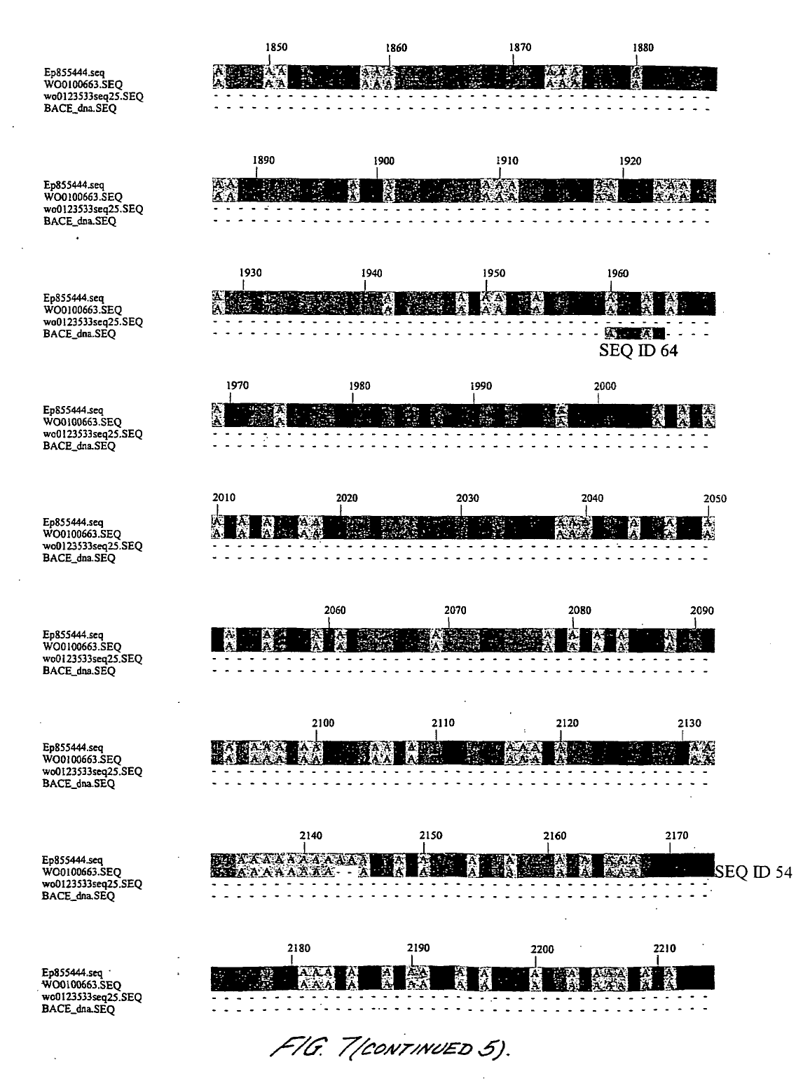

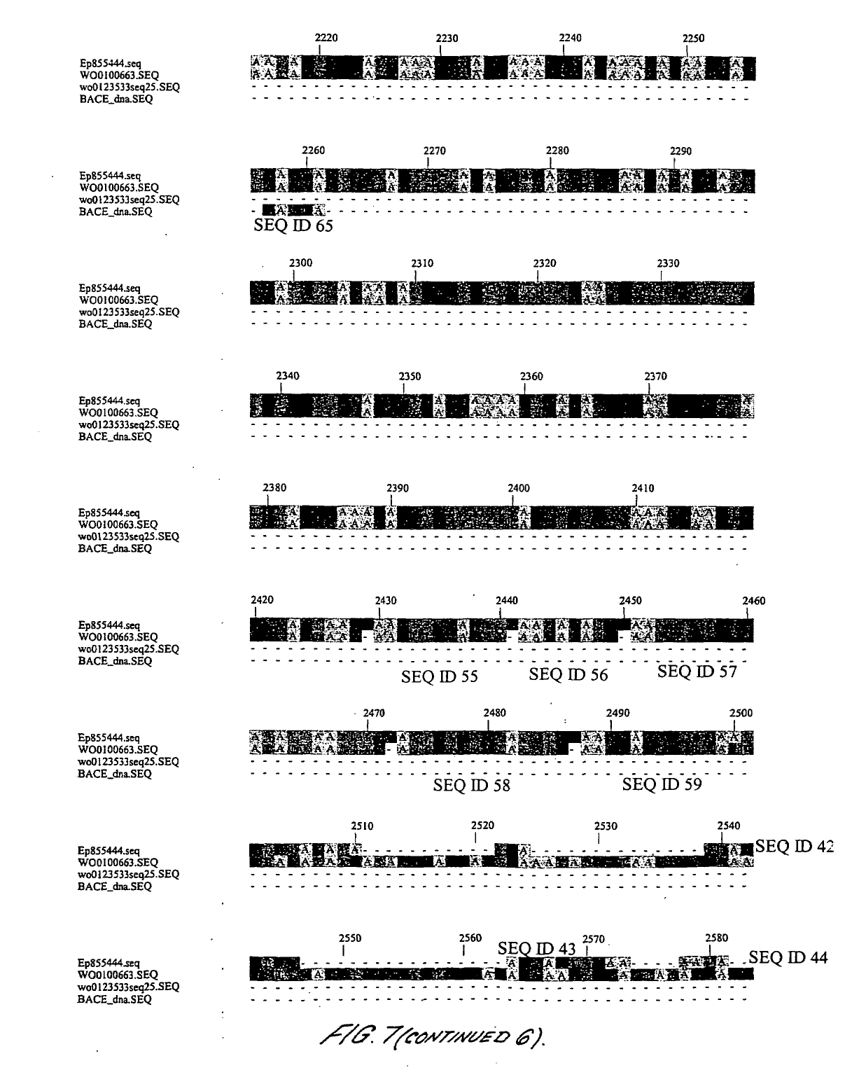

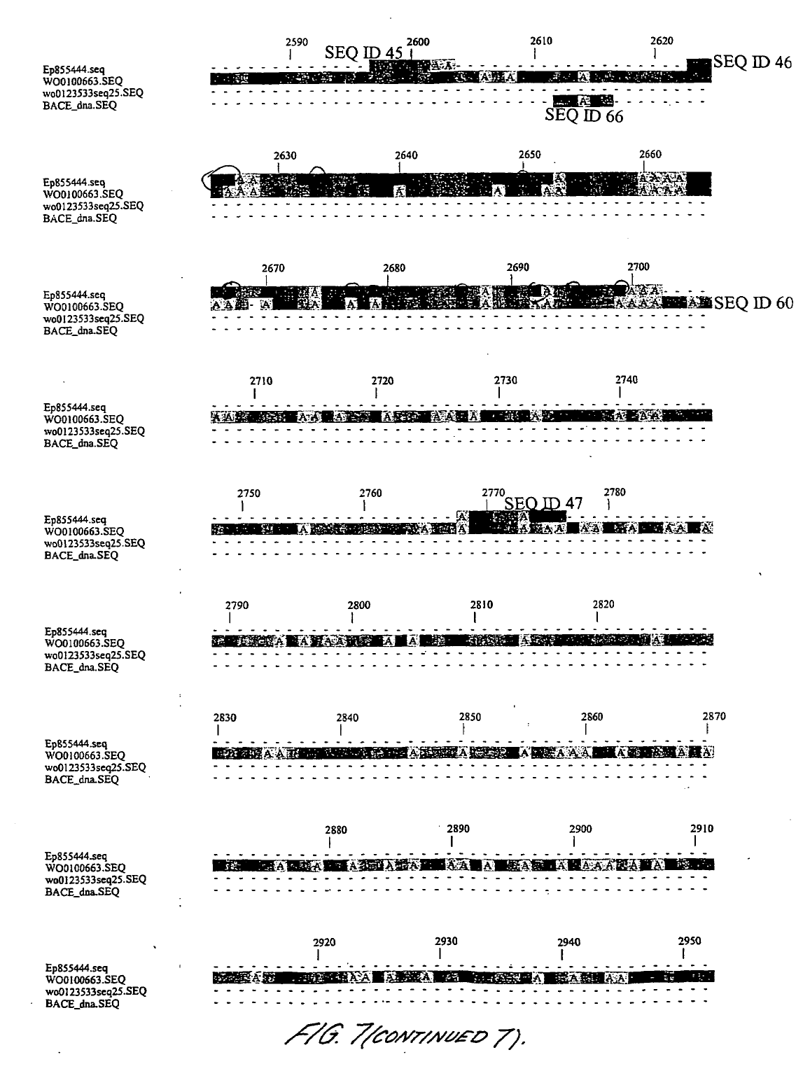

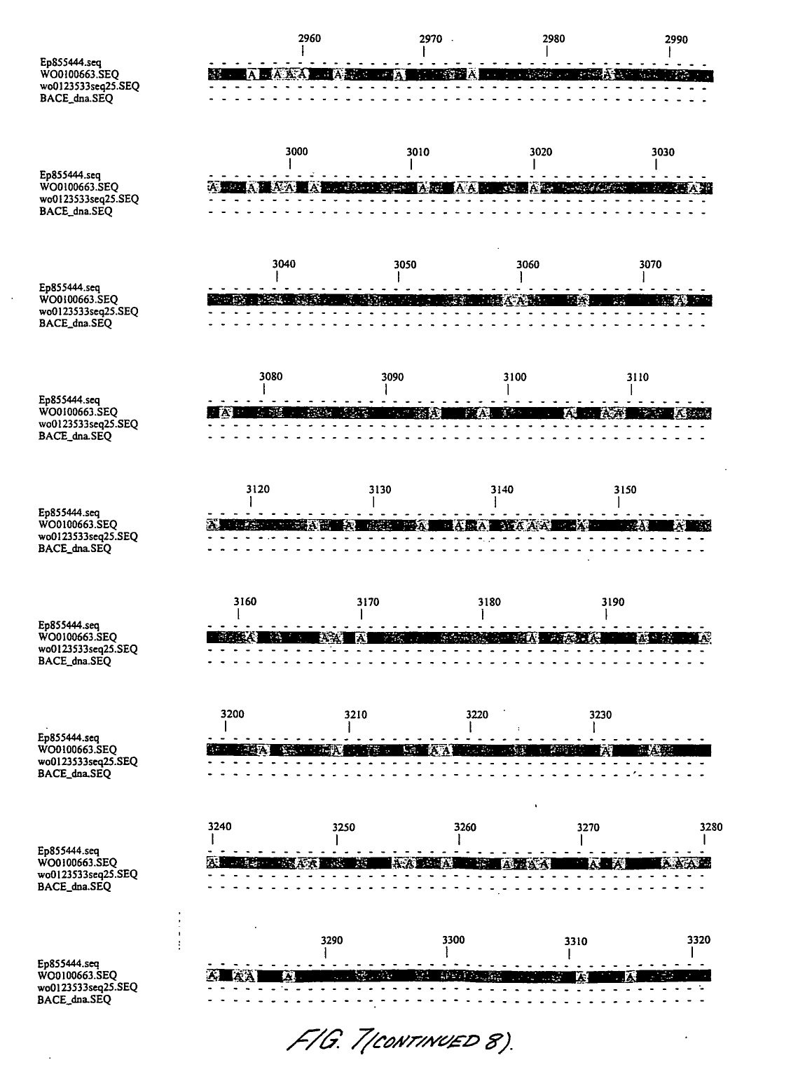

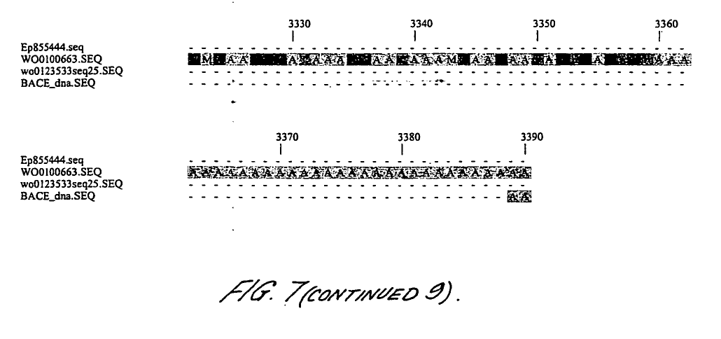

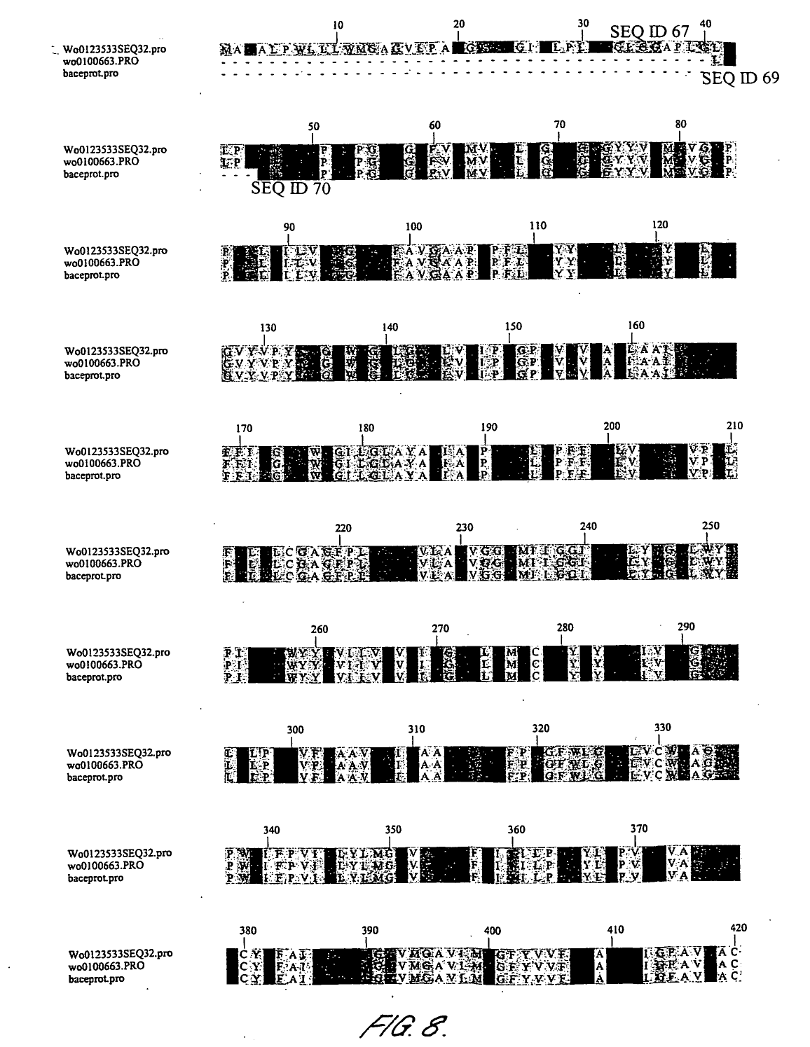

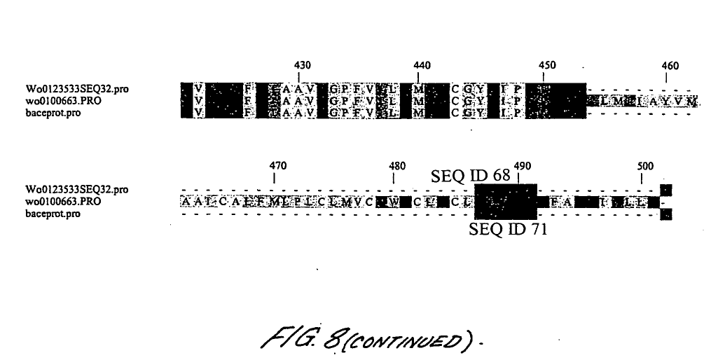

Both forms of the protein could be detected using an anti-His₆ antibody (see FIG. 5); only the unprocessed form containing the pro-peptide was detected using an anti-FLAG antibody. Further changes to the synthetic BACE catalytic domain sequence were the addition of the baculoviral gp67 signal sequence instead of the BACE signal, the addition of a FLAG tag to the N-terminus of the pro-peptide. The gp67 signal sequence increased the secretion of the protein into the cell culture medium, and the FLAG tag was added to allow differentiation between species arising from incomplete pro-peptide cleavage (and to determine if separation is required) (see FIG. 6): Insect cells infected with the BACE baculovirus secreted a mixture of processed and unprocessed BACE into the culture medium. FIG. 2A shows the polypeptide sequence encoded by the synthetic BACE gene.

As mentioned previously, the invention comprehends the use of the inventive BACE proteins in assays or methods for determining inhibitors thereof, e.g., compounds, compositions or active agents or ingredients that bind to BACE, advantageously irreversibly, preferably so as to have a therapeutic effect with respect to AD and other maladies. After determination of a suitable compound, composition, active agent or ingredient that binds to BACE, the compound, composition, active agent or ingredient is then formulated into a composition for administration and is administered to a subject in need thereof. These therapeutics can be administered in known formulations, by known routes of administration, following the teachings of documents cited herein.

It is noted that these therapeutics can be a chemical compound and/or antibody and/or portion thereof or a pharmaceutically acceptable salt and can be administered alone or as an active ingredient in combination with pharmaceutically acceptable carriers, diluents, and vehicles, as well as other active ingredients.

The compounds can be administered orally, subcutaneously or parenterally including intravenous, intraarterial, intramuscular, intraperitoneally, and intranasal administration as well as intrathecal and infusion techniques.

It is noted that humans are treated generally longer than the mice or other experimental animals which treatment has a length proportional to the length of the disease process and drug effectiveness. The doses may be single doses or multiple doses over a period of several days, but single doses are preferred. Thus, one can scale up from animal experiments, e.g., rats, mice, and the like, to humans, by techniques from this disclosure and documents cited herein and the knowledge in the art, without undue experimentation.

The treatment generally has a length proportional to the length of the disease process and drug effectiveness and the patient being treated.

When administering a therapeutic of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various amounts of the other ingredients, as desired.

A pharmacological formulation of the present invention, e.g., comprising a therapeutic compound, can be administered to the patient in an injectable formulation containing any compatible carrier, such as various vehicles, adjuvants, additives, and diluents; or the compounds utilized in the present invention can be administered parenterally to the patient in the form of slow-release subcutaneous implants or targeted delivery systems such as monoclonal antibodies, iontophoretic, polymer matrices, liposomes, and microspheres.

A pharmacological formulation of the compound utilized in the present invention can be administered orally to the patient. Conventional methods such as administering the compounds in tablets, suspensions, solutions, emulsions, capsules, powders, syrups and the like are usable. Known techniques which deliver the compound orally or intravenously and retain the biological activity are preferred.

In one embodiment, a formulation of the present invention can be administered initially, and thereafter maintained by further administration. For instance, a formulation of the invention can be administered in one type of composition and thereafter further administered in a different or the same type of composition. For example, a formulation of the invention can be administered by intravenous injection to bring blood levels to a suitable level. The patient's levels are then maintained by an oral dosage form, although other forms of administration, dependent upon the patient's condition, can be used.

The quantity to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention. Typically, an adjuvant or additive is commonly used as 0.001 to 50 wt % solution in phosphate buffered saline, and the active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %. Of course, for any composition to be administered to an animal or human, and for any particular method of administration, it is preferred to determine therefor: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response, such as by titrations of sera and analysis thereof. Such determinations do not require undue experimentation from the knowledge of the skilled artisan, this disclosure and the documents cited herein. And, the time for sequential administrations can be ascertained without undue experimentation.

Examples of compositions comprising a therapeutic of the invention include liquid preparations for orifice, e.g., oral, nasal, anal, vaginal, peroral, intragastric, mucosal (e.g., perlingual, alveolar, gingival, olfactory or respiratory mucosa) etc., administration such as suspensions, syrups or elixirs; and, preparations for parenteral, subcutaneous, intradermal, intramuscular or intravenous administration (e.g., injectable administration), such as sterile suspensions or emulsions. Such compositions may be in admixture with a suitable carrier, diluent, or excipient such as sterile water, physiological saline, glucose or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Compositions of the invention, are conveniently provided as liquid preparations, e.g., isotonic aqueous solutions, suspensions, emulsions or viscous compositions which may be buffered to a selected pH. If digestive tract absorption is preferred, compositions of the invention can be in the “solid” form of pills, tablets, capsules, caplets and the like, including “solid” preparations which are time-released or which have a liquid filling, e.g., gelatin covered liquid, whereby the gelatin is dissolved in the stomach for delivery to the gut. If nasal or respiratory (mucosal) administration is desired, compositions may be in a form and dispensed by a squeeze spray dispenser, pump dispenser or aerosol dispenser. Aerosols are usually under pressure by means of a hydrocarbon. Pump dispensers can preferably dispense a metered dose or, a dose having a particular particle size.

Compositions of the invention can contain pharmaceutically acceptable flavors and/or colors for rendering them more appealing, especially if they are administered orally. The viscous compositions may be in the form of gels, lotions, ointments, creams and the like (e.g., for transdermal administration) and will typically contain a sufficient amount of a thickening agent so that the viscosity is from about 2500 to 6500 cps, although more viscous compositions, even up to 10,000 cps may be employed. Viscous compositions have a viscosity preferably of 2500 to 5000 cps, since above that range they become more difficult to administer. However, above that range, the compositions can approach solid or gelatin forms which are then easily administered as a swallowed pill for oral ingestion.

Liquid preparations are normally easier to prepare than gels, other viscous compositions, and solid compositions. Additionally, liquid compositions are somewhat more convenient to administer, especially by injection or orally. Viscous compositions, on the other hand, can be formulated within the appropriate viscosity range to provide longer contact periods with mucosa, such as the lining of the stomach or nasal mucosa.

Obviously, the choice of suitable carriers and other additives will depend on the exact route of administration and the nature of the particular dosage form, e.g., liquid dosage form (e.g., whether the composition is to be formulated into a solution, a suspension, gel or another liquid form), or solid dosage form (e.g., whether the composition is to be formulated into a pill, tablet, capsule, caplet, time release form or liquid-filled form).

Solutions, suspensions and gels, normally contain a major amount of water (preferably purified water) in addition to the active compound. Minor amounts of other ingredients such as pH adjusters (e.g., a base such as NaOH), emulsifiers or dispersing agents, buffering agents, preservatives, wetting agents, jelling agents, (e.g., methylcellulose), colors and/or flavors may also be present. The compositions can be isotonic, i.e., it can have the same osmotic pressure as blood and lacrimal fluid.

The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions may be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

A pharmaceutically acceptable preservative can be employed to increase the shelf-life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight although there may be appreciable variation depending upon the agent selected.

Those skilled in the art will recognize that the components of the compositions should be selected to be chemically inert with respect to the active compound. This will present no problem to those skilled in chemical and pharmaceutical principles, or problems can be readily avoided by reference to standard texts or by simple experiments (not involving undue experimentation), from this disclosure and the documents cited herein.

The inventive compositions of this invention are prepared by mixing the ingredients following generally accepted procedures. For example the selected components may be simply mixed in a blender, or other standard device to produce a concentrated mixture which may then be adjusted to the final concentration and viscosity by the addition of water or thickening agent and possibly a buffer to control pH or an additional solute to control tonicity. Generally the pH may be from about 3 to 7.5. Compositions can be administered in dosages and by techniques well known to those skilled in the medical and veterinary arts taking into consideration such factors as the age, sex, weight, and condition of the particular patient, and the composition form used for administration (e.g., solid vs. liquid). Dosages for humans or other mammals can be determined without undue experimentation by the skilled artisan, from this disclosure, the documents cited herein, and the knowledge in the art.

Suitable regimes for initial administration and further doses or for sequential administrations also are variable, may include an initial administration followed by subsequent administrations; but nonetheless, may be ascertained by the skilled artisan, from this disclosure, the documents cited herein, and the knowledge in the art.

Accordingly, the invention comprehends, in further aspects, methods for preparing therapeutic compositions including an active agent, ingredient or compound or BACE inhibitor as from inventive methods herein for ascertaining compounds that bind to and/or inhibit BACE, as well as to methods for inhibiting BACE or the production of Aβ or fragments thereof or treating AD or other maladies.

Furthermore, as discussed herein, the inventive BACE proteins are useful in generating antibodies, which are themselves useful in assays as well as in therapeutics. From documents cited herein, one can readily make and use anti-BACE antibodies and methods for producing monoclonal antibodies are well known to those of ordinary skill in the art, see, e.g., U.S. Pat. Nos. 4,196,265 and 6,221,645. Thus, the BACE proteins of the invention can be used to generate antibodies and the antibodies can be used, without undue experimentation.

The invention will now be further described by the following non-limiting Examples, given by way of illustration.

### EXAMPLES

### Example 1

### Production of BACE in Insect Cells

A. Gene Construction and Cloning

The synthetic BACE catalytic domain sequence was constructed a combination of oligonucleotide synthesis and overlap PCR (Cambridge Bioscience Ltd, Cambridge UK). Mutations were inserted at specific sites within the BACE catalytic domain sequence during synthesis to reduce the GC content of the gene. The synthetic gene was then cut with restriction enzymes Sal1 and Not1 to generate a 1489 bp fragment which was then subcloned into the expression vector pFastBac1 (LifeTechnologies), and the DNA sequence verified by standard DNA sequencing methods (e.g., electrophoresis and automated DNA sequence analysis of the insert).

The cDNA encoding human furin was cut by restriction enzymes to generate a 3216 bp Sma1 Xma1 fragment that was then subcloned into the expression vector pFastBac Dual (LifeTechnologies).

B. Baculovirus Generation and Fermentation

Recombinant baculoviruses were constructed by using the expression vectors of the Bac-to-Bac™ system (LifeTechnologies), according to the manufacturers instructions. Manipulations involving insect cells and baculoviruses were carried out according to standard protocols (King and Possee, 1992).

As it has been shown that coexpression of the prohormone convertase furin increases expression of mature TGF-β in insect cells (Laprise et al., 1998), the effect of furin co-expression on BACE production was evaluated i.e., while the total amount of BACE produced did not increase with furin coexpression, there was a reproducible increase in the fraction of processed protein from about 30% of total BACE up to about 60%; this result is quite surprising and advantageous.

Trichoplusia ni HighFive cells (Invitrogen, Carlsbad Calif., USA) were found to give higher levels of BACE expression than Spodoptera frugiperda Sf9 cells, and were used for all protein production. Protein production was carried out in a 20-30 liter working volume bioreactor (Applikon Dependable Instruments, Schiedam, Netherlands), containing Excell 405 medium (JRH Scientific). Cells were infected at a multiplicity of infection (MOI) of 0.1 of each virus at a cell density of 1.5×10⁶ cells/ml. Glucose concentration was measured during the fermentation and adjusted to maintain the starting concentration. Three days after viral infection the HighFive cells were cleared from the medium by continuous flow centrifugation and the medium was concentrated approximately 30-fold by ultrafiltration.

C. Purification of BACE

The expressed BACE protein was purified by affinity chromatography on nickel agarose resin. Initially, the concentrated medium containing the expressed BACE protein was dialysed overnight against 50 mM sodium phosphate pH 8.0, 50 mM sodium acetate, 300 mM NaCl and 10 ml Ni-NTA agarose resin (Qiagen) and equilibrated in the above buffer. Imidazole (Sigma) was added to a final concentration of 5 mM, Pefabloc (Roche Molecular Biochemicals, Lewes, UK) was added to 0.1 g/L and the sample was mixed gently overnight at 4° C. The nickel agarose resin was then loaded onto an empty column and washed with 50 mM sodium phosphate pH 8.0, 300 mM NaCl until the absorption at 280 nm reached the baseline level of the above-mentioned buffer. The column was then washed with 4 column volumes of 50 mM sodium phosphate pH 8.0, 50 mM NaCl, 15 mM imidazole. The BACE protein was then eluted with a linear imidazole concentration gradient, five column volumes in size, from 50 mM sodium phosphate pH 8.0, 50 mM NaCl to 50 mM sodium phosphate pH 8.0, 50 mM NaCl, 300 mM imidazole, typically resulting in an absorption peak at 280 nm, corresponding to the BACE protein and other co-purified contaminating proteins.

After Nickel chromatography the BACE protein was purified by anion exchange chromatography, fractions corresponding to the BACE protein containing the peak were buffer exchanged on a XK-50 column (Amersham Pharmacia Biotech) containing 200 ml sephacryl S-200, into 25 mM Tris pH 8.1, 5 mM NaCl (Anion loading buffer) and then loaded onto a Resource Q anion exchange column (Amersham Pharmacia Biotech). The protein was eluted with a 35 column volume linear salt gradient from 100% loading buffer to 100% elution buffer (25 mM Tris pH8.1, 400 mM NaCl). Fractions were pooled based on analysis by SDS-PAGE.

The pooled fractions were dialysed against HIC loading buffer: 50 mM Tris pH 8.1, 50 mM NaCl, 0.9 M (NH₄)₂SO₄. The final sample was then loaded onto a HIC column (Source PHE, Amersham Pharmacia Biotech) equilibrated with HIC loading buffer, and washed to a stable baseline with loading buffer. The differentially processed forms of the BACE generated by proteolytic activity were eluted as separate peaks using a 35 column volume gradient from loading buffer to 50 mM Tris pH 8.1, 50 mM NaCl.

Peak fractions containing the required form of BACE protein were pooled based on analysis by SDS-PAGE and dialysed against 50 mM HEPES pH 8.0, 100 mM NaCl, 1 Mm DTT.

The dialysed sample was concentrated to a 12 ml volume and loaded immediately onto a Sephacryl S-200 column (Amersham Pharmacia Biotech) pre-equilibrated with 50 mM HEPES pH 8.0, 100 mM NaCl, 1 Mm DTT and stored at 4° C. after elution.

The purified BACE eluted from the size exclusion column at the position expected for monomeric protein and was monodisperse when subjected to dynamic light scattering. SDS-PAGE showed the representative samples after the different column steps is shown in FIG. 5.

### Example 2

### Crystallization of BACE

A. Crystallization

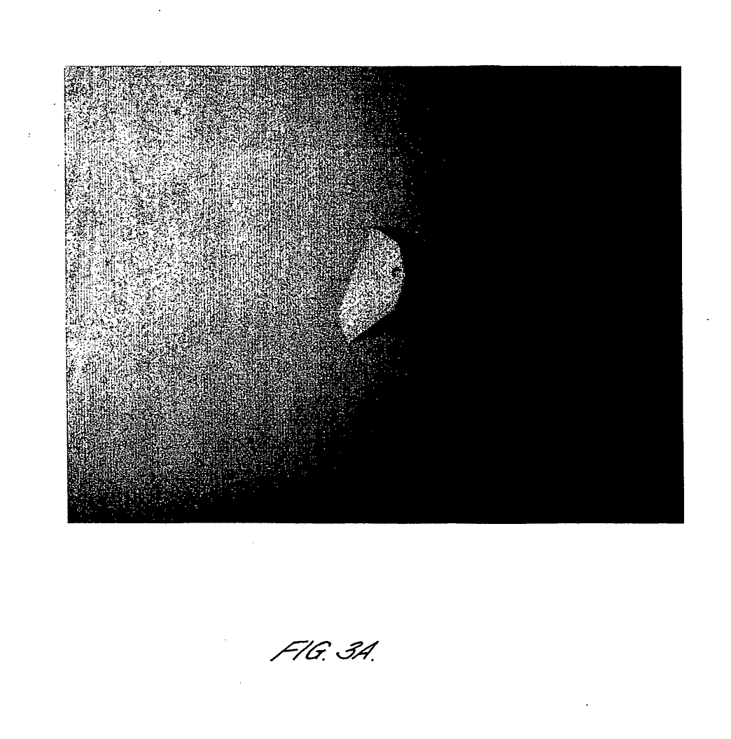

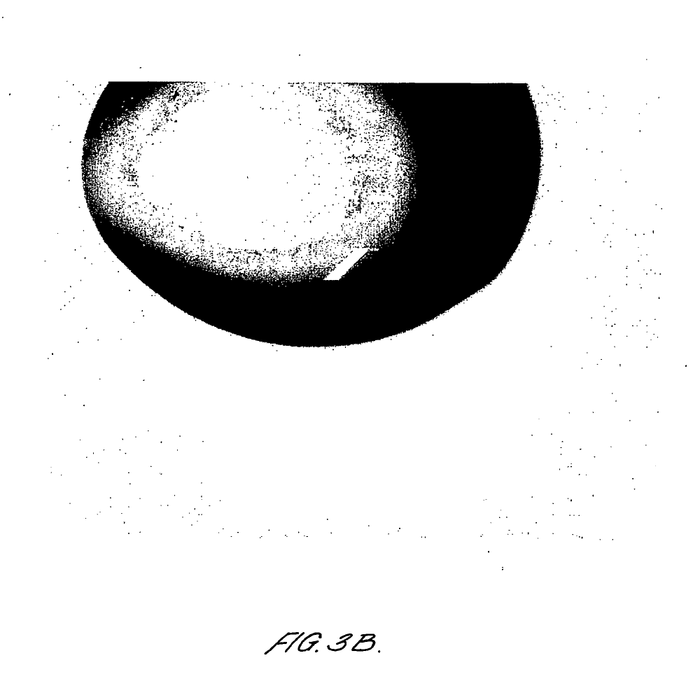

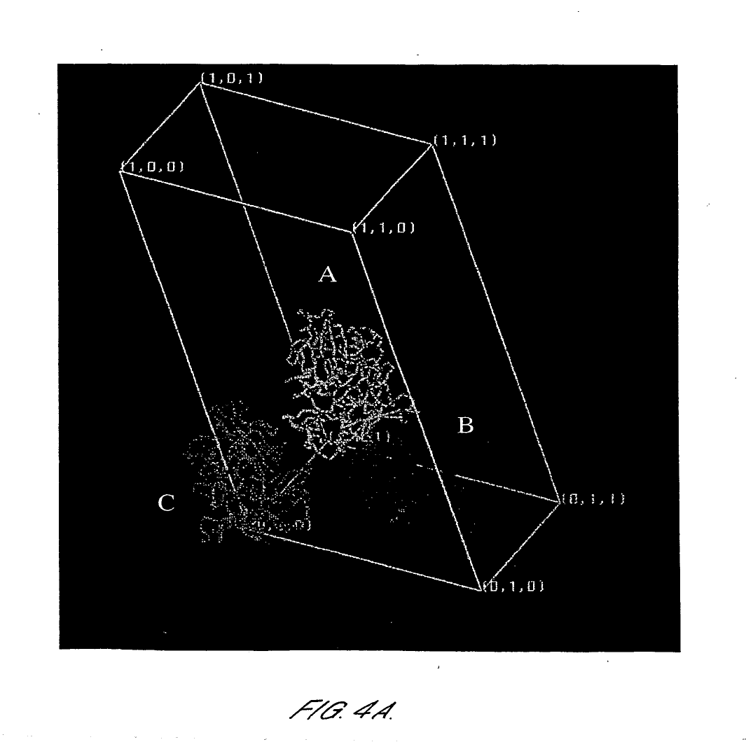

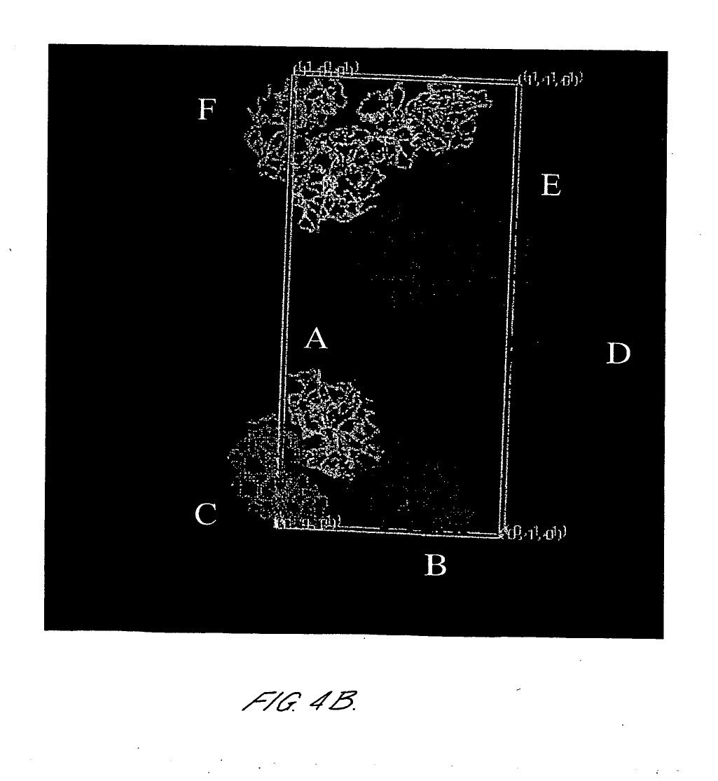

Crystals of BACE were grown by the hanging drop vapor diffusion method, in which 1 μl of protein solution and 1 μl of well solution (100 mM Tri-sodium citrate, pH 5.8, 200 mM ammonium iodide and 18-20% PEG monomethyl ether, 5K) were placed on a cover slip and equilibrated over 1 ml of well solution at 20° C. The protein concentration was 5 mg/ml in 50 mM HEPES, pH 8.0, 150 mM NaCl, 1 mM DTT. Small prismatic crystals appeared after two days and grew to a maximum size of 0.2 mm×0.1 mm×0.1 mm after two weeks. (FIGS. 3A and B).

Crystals of BACE complexed with OM99-2 (Ghosh et al., 2000) were grown using a similar method. BACE, at a concentration of 0.2 mg/ml was mixed with an excess of inhibitor and kept at 4° C. for 1 hour. The BACE protein was then concentrated to 5 mg/ml using a centricon column with a molecular weight cutoff of 10000, and the crystallization drops set up as before. Crystals with the same morphology as the uncomplexed enzyme appeared after two days and grew to a maximum size of 0.25 mm×0.1 mm×0.1 mm.

Both BACE with no inhibitor and BACE in the presence of OM99-2 formed crystals belonging to space group C2. The cell dimensions for the crystals grown in the presence of OM99-2 (FIG. 3A) were a=236.63 Å, b=105.02 Å, c=62.59 Å and β=101.32°, and the asymmetric unit of the crystal contained 3 copies of BACE. The cell dimensions for the crystals grown in the absence of any inhibitor (FIG. 3B) were a=238.3 Å, b=107.4 Å, c=60.4 Å, b=101.89°.

Apo-Soaked Crystal Experimental: Crystals of BACE, grown in the absence of inhibitor as previously described were soaked in a solution of inhibitor for 1 hour. The inhibitor was previously dissolved in DMSO to a concentration of 10 mM and then diluted 1 in 10 in the well solution as previously described. 20 microliters of this was placed in a microbridge, and an apo BACE crystal added to it. The microbridge was sealed and incubated for 3.5 hours.

B. Data Collection and Processing

The structure of BACE as a complex with OM99-2 was solved to 2.6 Å using the method of molecular replacement. Data was collected at 100K on crystals frozen in a solution containing a suitable cryoprotectant. The cryoprotectant solution consisted of 100 mM Tri-sodium citrate, pH 5.8, 200 mM ammonium iodide, 15% PEG monomethyl ether 5K, and 20% PEG 400. The crystal was immersed in the cryoprotectant solution for 30 seconds prior to freezing in liquid nitrogen for the purposes of storage. Data was collected to 2.6 Å on beamline ID14-2 at the European Synchrotron Radiation Facility using a MARCCD detector, with a wavelength of 0.934 Å and processed using D*trek (Pflugrath, J., 1999). The dataset was scaled using SCALA and the intensities converted to structure factors using TRUNCATE, from the CCP4 suite of programs (Collaborative Computing Project, 1994). Statistics for the processed data are listed in Table 1.

TABLE 1
Data collection statistics for BACE crystallized as a complex
with OM99-2.

| Resolution | 2.6 Å |
|---|---|
| Mosaicity | 0.8° |
| Completeness | 95.4% |
| Multiplicity | 1.96 |
| Rmerge | 0.087 |

This table shows that the experimental data used to solve the structure of the BACE/OM99-2 complex was of good quality and sufficient completeness to enable a reliable structure to be derived from it.

Apo-Crystal Experimental: The soaked crystal was then removed, dipped in a solution containing a cryoprotectant mixed with the inhibitor in DMSO, in the same proportions as previously (100 mM Tri-sodium citrate, pH 5.8, 200 mM ammonium iodide, 15% PEG monomethyl ether 5K and 20% PEG 400). The crystal was then frozen as for the OM99-2 crystals and data collected. Data was collected on station ID14-1 at the ESRF using an ADSC detector.

C. Structure Determination and Refinement

The structure of the BACE/OM99-2 complex was solved by molecular replacement using the program AMORE (Navaza, 1994). The molecular replacement solution was not as straightforward application of AMORE. Rather, it involved the use of CCP4, the programs POLARRFN and RFCORR, as well as inventive effort, e.g., to so use this combination and especially to so use RFCORR (Collaborative Computing Project, 1994). The search model was the A chain of 1FKN (Hong et al. 2000) taken from the pdb database (1FKN.pdb); a search radius of 35 Å and a resolution range of 8.0-3.0 Å being used to give a solution with an Rfactor of 0.38 and a correlation coefficient of 0.714. This solution was used as a starting point for refinement using the program REFMAC5, also from the CCP4 suite of programs (Collaborative Computing Project, 1994). The inhibitor, OM99-2 was absent from the initial model, and convincing electron density in the active site of all three copies of BACE was observed in difference Fourier maps. This provided confirmation that the solution to the molecular replacement was correct. Cycles of refinement of the structure were alternated with manual rebuilding of the model using QUANTA (1994, San Diego, Calif.: Molecular Simulations). The N- and C-termini of the molecule were rebuilt, asparagine residues 153, 172, 223 and 354 were remodeled as glutamine residues. The inhibitor molecule was built into the electron density with QUANTA (1994, San Diego, Calif.: Molecular Simulations), and finally the water molecules were added using DenInt (Astex internal software library). Refinement statistics are shown in Table 2 Å.

Data collected with apo crystals soaked with inhibitor were processed using D*Trek (Pflugrath, J., 1999), and the intensities converted to structure factors using TRUNCATE, from the CCP4 suite of programs (Collaborative Computing Project, 1994).

The space group of these crystals has changed from the apo form (space group C2) to P2, with cell dimensions a=62.8, b=106.8 Å, c=227.9 Å and β=93.63°. The statistics are given in Table 2B below

The structure of the soaked BACE/inhibitor complex was solved by molecular replacement using the programs AMORE (Navaza, 1994). The search model was a monomer from the BACE/OM99-2 structure. A search radius of 35 Å and a resolution range of 12-4 Å gave a solution with and Rfactor of 0.421 and a correlation coefficient of 0.638. There are 6 monomers in the asymmetric unit. This solution was used as a starting point for refinement using the programs CNX (1999, San Diego, Calif.: Molecular Simulations) and BUSTER (Bricogne, 1993, Acta Cryst. D49, 37-60). The final refinement statistics are given in Table 2C below.

TABLE 2A
Final refinement statistics for The C2 BACE/OM99-2 complex.

| | |
|---|---|
| Rwork | 0.231 |
| Rfree | 0.312 |
| RMS bond deviation from ideality | 0.022 Å |
| RMS bond angle deviation from ideality | 2.4° |
| Average Bfactor for structure | 53.4 Å² |

This data indicates that the final structure is of good quality, the Rfactors indicating that the refined model has a good agreement with the experimental data. The RMS deviations from ideality indicate that the geometry of the model is good and in agreement with previous data.

TABLE 2B
Statistics for P2₁ BACE/inhibitor complex

| Resolution | 3.0 Å |
|---|---|
| Mosaicity | 0.45 |
| Completeness | 98% |
| Multiplicity | 2.3 |
| Rmerge | 13.8% |

The statistics show the space group has changed from the apo-BACE form (space group C2) to P2₁ with cell dimensions a = 62.8, b = 106.8 Å, c = 227.9 Å and β = 93.63°″

TABLE 2C
Final refinement statistics for P2₁ BACE/inhibitor complex.

| | |
|---|---|
| Rwork | 30.3% |
| Rfree | 34.5% |
| RMS bond deviation from ideality | 0.015 Å |
| RMS bond angle deviation from ideality | 1.027° |
| Average Bfactor for structure | 35.6 Å |

### Results and Discussion

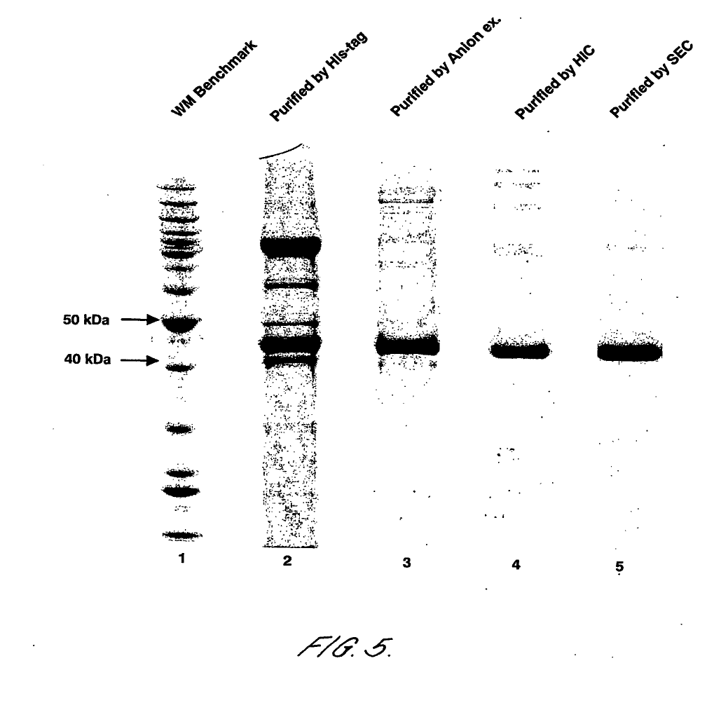

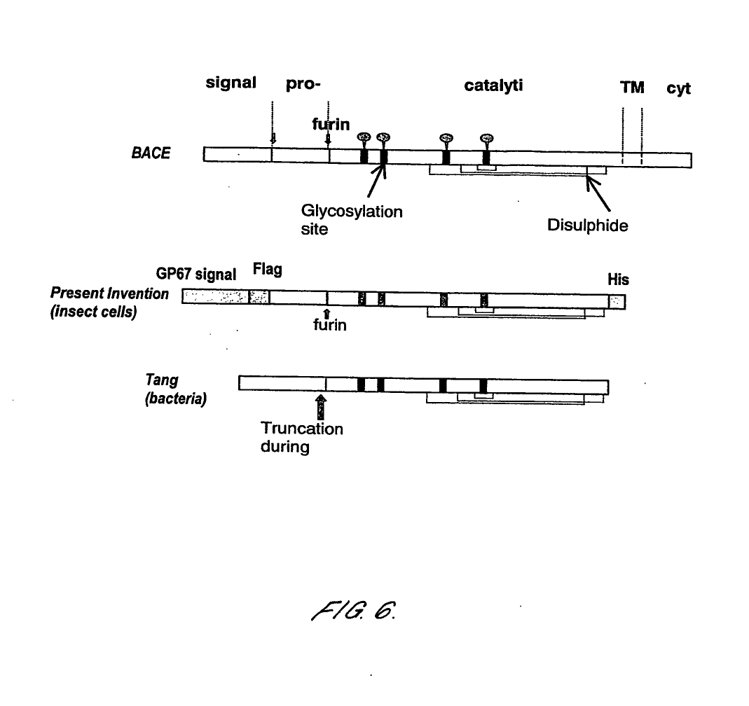

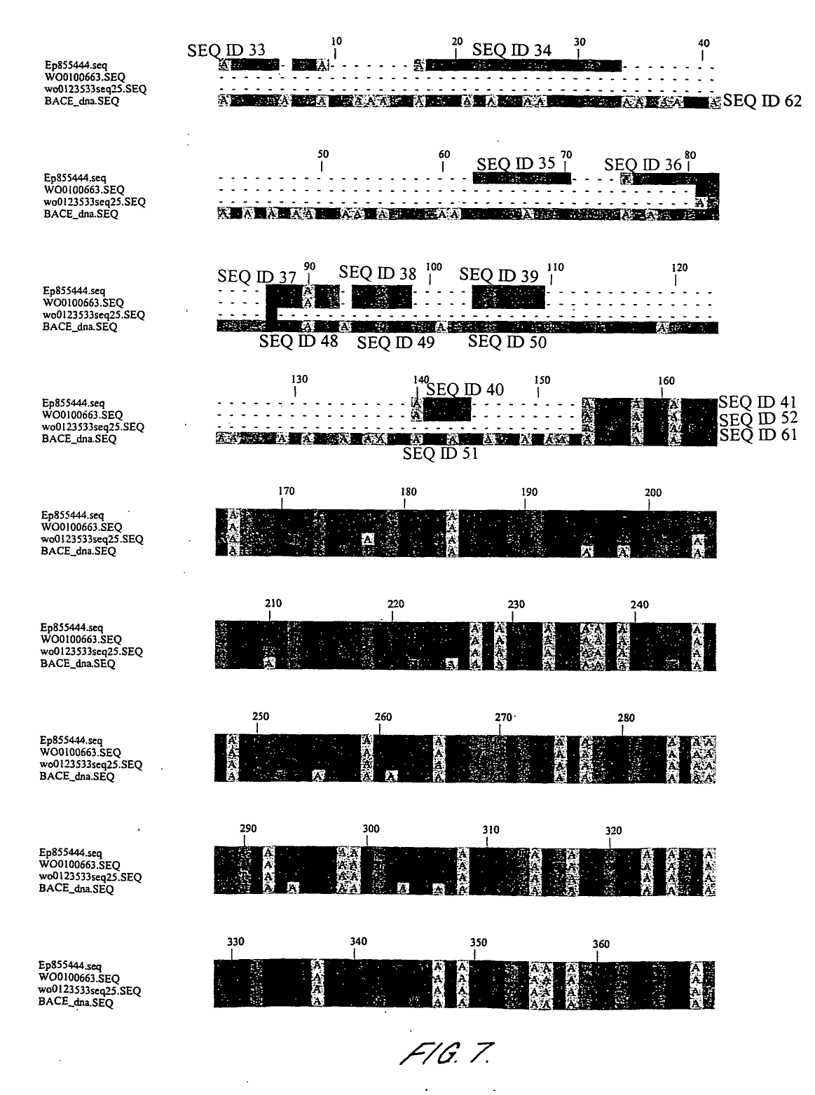

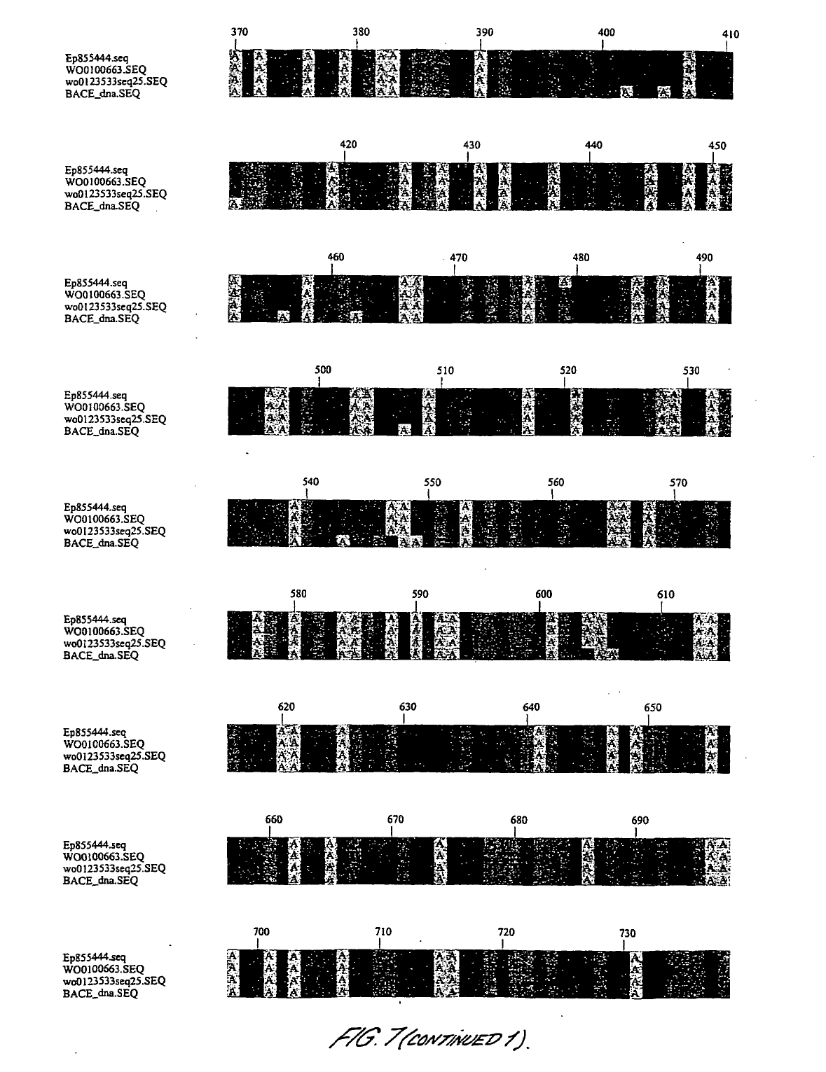

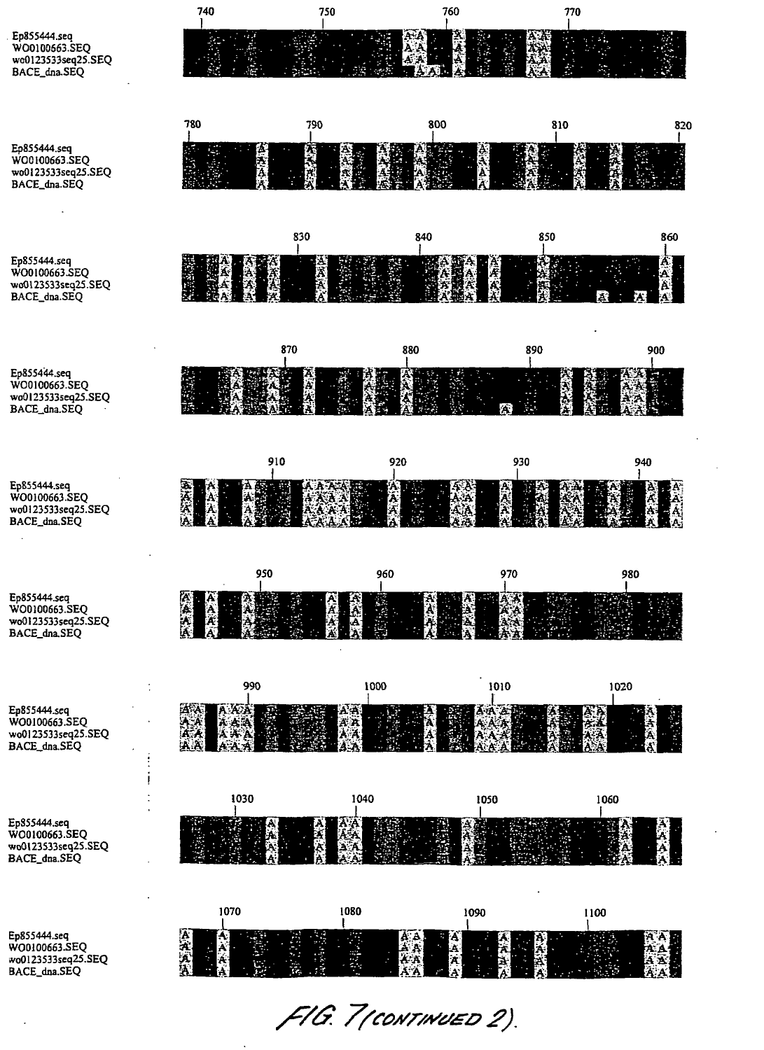

The final model of the C2 crystal structure of BACE/OM99-2 contained 1161 residues in 3 protein molecules, 3 copies of OM99-2 and 183 ordered water molecules, an Rfactor of 0.231 and a free Rfactor of 0.312. The asymmetric unit contained 3 copies of the BACE molecule (FIGS. 4A and B), A, B and C, two of which, B and C, form a dimer related by a non-crystallographic two-fold axis. Molecule A forms a similar dimer with its crystallographically related molecule A in an adjacent asymmetric unit. The positions of residues −2 to 385 of all three independent molecules are well defined by the electron density. There is no evidence of density beyond serine −2 for any of the molecules, and the N-terminus of molecules A and C interact with OD1 of asparagine 98 in symmetry related molecules B and A respectively. The N-terminus of molecule B is in a region of solvent. This lack of electron density for all three molecules indicates that the other residues from the N-terminus have no ordered structure. The electron density at the C-terminus ends with residue Asn 385, with no evidence for further terminal residues for the His-tag.

The bilobal structure of individual molecules of BACE as solved in the C2 crystal form, is essentially the same as that of memapsin 2 as solved in the P2₁ crystal form. The interactions made by the specific mutations are shown in Table 3. These include the N and C-terminus and the Asn [symbol] Gln mutations in each of the three independent units. The mutation of Asn111 to Gln111 appears to be important in the formation of the crystals in that Gln111 of molecule B lies close to the crystallographic two-fold axis and interacts with the symmetry related B:111. Difference electron density was initially seen for OM99-2 in all three molecules, and the inhibitor molecule was fitted to this. Its position was well defined from P4 to P4′ for molecules A and C P4′ was less well defined in molecule B. The active site is open to solvent for molecules B and C, but that of molecule A is partially occluded (close to P4′) by a symmetry related molecule C.

TABLE 3
Interactions made by mutated residues in BACE. Monomers
A B and C are described in column 1 and the residues with
which they interact in column 2.

| Mutated or different | |
|---|---|
| residue (from | Interactions formed by mutated or different |
| Tang sequence) | residue. |
| A: 92 | B: 142, a crystallographic symmetry related lysine |
| A: 111 | No direct interactions |
| A: 162 | No direct interactions |
| A: 293 | No direct interactions |
| B: 92 | No direct interactions |
| B: 111 | B: 111 symmetry related glutamine, lies on 2-fold |
| B: 162 | Close to crystallographic symmetry axis, but no |
| | direct interactions |
| B: 293 | Points towards solvent channel |
| C: 92 | A: 142 a crystallographic symmetry related lysine |
| C: 111 | No direct interactions |
| C: 162 | No direct interactions |
| C: 293 | No direct interactions |
| A: Nterm | Nterm makes interaction with symmetry related |
| | OD1 of Asn98B |
| B: Nterm | Nothing beyond residue ser-2 |
| C: Nterm | N-term makes interaction with symmetry related |
| | OD1 of Asn98A |
| A: Cterm | No interactions |
| B: Cterm | No interactions |
| C: Cterm | No interactions. |

This table shows that most of the mutations made to the BACE enzyme are at positions which do not affect the crystal packing. The major exception is in the case of residue B111, which is shown to interact across the crystallographic two-fold axis with its symmetry related molecule.

TABLE 4
Comparison of Tang/Hong structure with present invention.

| Measurement | Tang/Hong | Present Invention |
|---|---|---|
| Inhibitor OM99-2 | Crystallized in presence of | Crystallized in presence and |
| | inhibitor OM99-2 | absence of OM99-2. |
| Crystallisation conditions | 5 mg/ml protein, 5-fold molar | 6 mg/ml protein, excess |
| | excess of OM99-2 | OM99-2, 0.2M ammonium |
| | 0.2M ammonium sulphate, 22.5% | iodide, 20% PEGMME5K, |
| | PEG8K, 0.1M Na-cacodylate, | 0.1 M tri-sodium citrate, pH |
| | pH 7.4 | 5.6-5.8 |
| Space Group | P2₁ | C2 |
| Cell dimensions(Å) | a = 53.7, b = 85.9, 109.2, beta = 101.4° | a = 236.6, b = 105.0, c = 62.59, |
| | | beta = 101.3° |
| Resolution | 1.9 Å | 2.6 Å |
| pH of crystallization | 7.4 | 5.8 |
| Molecules in asymmetric | 2 | 3 |
| unit | | |
| N-terminus | −28p | −33p |
| C-terminus | No His-tag | His-tag |
| Mutations | None known | Asn→Gln (glycosylation |
| | | sites) |
| Dimer interface | Around N209 | Same dimer interface |
| B: 111 environment | solvent exposed | interact across 2-fold axis. |
| Accessibility of active site | Both molecules accessible | 1 accessible, 1 inaccessible, |
| | | 1 partly accessible |
| OM99-2 binding mode | As described in Science, 2000 | Binding modes similar, but |
| | 290 150-153 | P4′ is more ordered. |

This further demonstrates that the present invention is novel, nonobvious and inventive over the Tang PCT publications and the Hong Science article.

The following Table 5 provides atomic co-ordinates of BACE.

TABLE 5
Atomic co-ordinates.

| | | | |
|---|---|---|---|
| HEADER | ---- | XX-XXX-XX | xxxx |

| | |
|---|---|
| COMPND | --- |

| REMARK | 3 | | | | |
|---|---|---|---|---|---|
| REMARK | 3 | REFINEMENT. | | | |
| REMARK | 3 | PROGRAM : REFMAC 5.0 | | | |
| REMARK | 3 | | | | |
| REMARK | 3 | | | | |
| REMARK | 3 | REFINEMENT TARGET: MAXIMUM LIKELIHOOD | | | |
| REMARK | 3 | | | | |
| REMARK | 3 | DATA USED IN REFINEMENT. | | | |
| REMARK | 3 | RESOLUTION RANGE HIGH | (ANGSTROMS) | : | 2.60 |
| REMARK | 3 | RESOLUTION RANGE LOW | (ANGSTROMS) | : | 119.52 |
| REMARK | 3 | DATA CUTOFF | (SIGMA(F)) | : | NONE |
| REMARK | 3 | COMPLETENESS FOR RANGE | (%) | : | 93.40 |
| REMARK | 3 | NUMBER OF REFLECTIONS | | : | 40971 |
| REMARK | 3 | | | | |

| REMARK | 3 | FIT TO DATA USED IN REFINEMENT. | | | |
|---|---|---|---|---|---|
| REMARK | 3 | CROSS-VALIDATION METHOD | | : | THROUGHOUT |
| REMARK | 3 | FREE R VALUE TEST SET SELECTION | | : | RANDOM |
| REMARK | 3 | R VALUE | (WORKING + TEST SET) | : | 0.23480 |
| REMARK | 3 | R VALUE | (WORKING SET) | : | 0.23064 |
| REMARK | 3 | FREE R VALUE | | : | 0.31230 |

| | | | | | |
|---|---|---|---|---|---|
| REMARK | 3 | FREE R VALUE TEST SET SIZE | (%) | : | 5.1 |
| REMARK | 3 | FREE R VALUE TEST SET COUNT | | : | 2184 |

| REMARK | 3 | | | | |
|---|---|---|---|---|---|
| REMARK | 3 | FIT IN THE HIGHEST RESOLUTION BIN. | | | |
| REMARK | 3 | TOTAL NUMBER OF BINS USED | | : | 20 |
| REMARK | 3 | BIN RESOLUTION RANGE HIGH | | : | 2.601 |
| REMARK | 3 | BIN RESOLUTION RANGE LOW | | : | 2.668 |
| REMARK | 3 | REFLECTION IN BIN | (WORKING SET) | : | 2799 |
| REMARK | 3 | BIN R VALUE | (WORKING SET) | : | 0.282 |

| | | | | |
|---|---|---|---|---|
| REMARK | 3 | BIN FREE R VALUE SET COUNT | : | 145 |
| REMARK | 3 | BIN FREE R VALUE | : | 0.403 |
| REMARK | 3 | | | |

| REMARK | 3 | NUMBER OF NON-HYDROGEN ATOMS USED IN REFINEMENT. | | |
|---|---|---|---|---|
| REMARK | 3 | ALL ATOMS | : | 9531 |

| | | | | | |
|---|---|---|---|---|---|
| REMARK | 3 | | | | |
| REMARK | 3 | B VALUES. | | | |
| REMARK | 3 | FROM WILSON PLOT | (A**2) | : | NULL |
| REMARK | 3 | MEAN B VALUE | (OVERALL, A**2) | : | 50.041 |

| REMARK | 3 | OVERALL ANISOTROPIC B VALUE. | | |
|---|---|---|---|---|
| REMARK | 3 | B11 (A**2) | : | −4.02 |
| REMARK | 3 | B22 (A**2) | : | −0.68 |
| REMARK | 3 | B33 (A**2) | : | 4.60 |
| REMARK | 3 | B12 (A**2) | : | 0.00 |
| REMARK | 3 | B13 (A**2) | : | 0.24 |
| REMARK | 3 | B23 (A**2) | : | 0.00 |

| | | | | | |
|---|---|---|---|---|---|
| REMARK | 3 | | | | |
| REMARK | 3 | ESTIMATED OVERALL COORDINATE ERROR. | | | |
| REMARK | 3 | ESU BASED ON R VALUE | (A) | : | 1.142 |
| REMARK | 3 | ESU BASED ON FREE R VALUE | (A) | : | 0.408 |
| REMARK | 3 | ESU BASED ON MAXIMUM LIKELIHOOD | (A) | : | 0.512 |
| REMARK | 3 | ESU FOR B VALUES BASED ON MAXIMUM LIKELIHOOD | (A**2) | : | 23.746 |
| REMARK | 3 | | | | |

| | | | | |
|---|---|---|---|---|
| REMARK | 3 | CORRELATION COEFFICIENTS. | | |
| REMARK | 3 | CORRELATION COEFFICIENT FO-FC | : | 0.925 |
| REMARK | 3 | CORRELATION COEFFICIENT FO-FC FREE | : | 0.866 |
| REMARK | 3 | | | |

| REMARK | 3 | RMS DEVIATIONS FROM IDEAL VALUES | | COUNT | | RMS | | WEIGHT |
|---|---|---|---|---|---|---|---|---|
| REMARK | 3 | BOND LENGTHS REFINED ATOMS | (A) : | 9582 | ; | 0.022 | ; | 0.021 |
| REMARK | 3 | BOND LENGTHS OTHERS | (A) : | 8445 | ; | 0.001 | ; | 0.020 |

| | | | | | | | | |
|---|---|---|---|---|---|---|---|---|
| REMARK | 3 | BOND ANGLES REFINED ATOMS | (DEGREES) : | 13011 | ; | 2.356 | ; | 1.945 |
| REMARK | 3 | BOND ANGLES OTHERS | (DEGREES) : | 19641 | ; | 0.973 | ; | 3.000 |
| REMARK | 3 | TORSION ANGLES, PERIOD 1 | (DEGREES) : | 1173 | ; | 6.207 | ; | 3.000 |
| REMARK | 3 | TORSION ANGLES, PERIOD 3 | (DEGREES) : | 1607 | ; | 21.762 | ; | 15.000 |

| | | | | | | | | |
|---|---|---|---|---|---|---|---|---|
| REMARK | 3 | CHIRAL-CENTER RESTRAINTS | (A**3) : | 1422 | ; | 0.121 | ; | 0.200 |
| REMARK | 3 | GENERAL PLANES REFINED ATOMS | (A) : | 10704 | ; | 0.003 | ; | 0.020 |
| REMARK | 3 | GENERAL PLANES OTHERS | (A) : | 2010 | ; | 0.001 | ; | 0.020 |

| | | | | | | | | |
|---|---|---|---|---|---|---|---|---|
| REMARK | 3 | NON-BONDED CONTACTS REFINED ATOMS | (A) : | 2679 | ; | 0.339 | ; | 0.300 |
| REMARK | 3 | NON-BONDED CONTACTS OTHERS | (A) : | 9125 | ; | 0.264 | ; | 0.300 |
| REMARK | 3 | NON-BONDED TORSION OTHERS | (A) : | 3 | ; | 0.093 | ; | 0.500 |
| REMARK | 3 | H-BOND (X . . . Y) REFINED ATOMS | (A) : | 834 | ; | 0.226 | ; | 0.500 |
| REMARK | 3 | H-BOND (X . . . Y) OTHERS | (A) : | 35 | ; | 0.161 | ; | 0.500 |
| REMARK | 3 | SYMMETRY VDW REFINED ATOMS | (A) : | 20 | ; | 0.299 | ; | 0.300 |
| REMARK | 3 | SYMMETRY VDW OTHERS | (A) : | 62 | ; | 0.395 | ; | 0.300 |
| REMARK | 3 | SYMMETRY H-BOND REFINED ATOMS | (A) : | 10 | ; | 0.351 | ; | 0.500 |
| REMARK | 3 | SYMMETRY H-BOND OTHERS | (A) : | 2 | ; | 0.229 | ; | 0.500 |
| REMARK | 3 | | | | | | | |

| REMARK | 3 | ISOTROPIC THERMAL FACTOR RESTRAINTS. | | COUNT | | RMS | | WEIGHT |
|---|---|---|---|---|---|---|---|---|
| REMARK | 3 | MAIN-CHAIN BOND REFINED ATOMS | (A**2) : | 5889 | ; | 3.453 | ; | 5.000 |
| REMARK | 3 | MAIN-CHAIN ANGLE REFINED ATOMS | (A**2) : | 9501 | ; | 5.002 | ; | 6.000 |
| REMARK | 3 | SIDE-CHAIN BOND REFINED ATOMS | (A**2) : | 3693 | ; | 4.504 | ; | 6.000 |
| REMARK | 3 | SIDE-CHAIN ANGLE REFINED ATOMS | (A**2) : | 3510 | ; | 6.644 | ; | 7.500 |

| REMARK | 3 | | | | | | | |
|---|---|---|---|---|---|---|---|---|
| REMARK | 3 | NCS RESTRAINTS STATISTICS | | | | | | |
| REMARK | 3 | NUMBER OF NCS GROUPS : NULL | | | | | | |
| REMARK | 3 | | | | | | | |
| REMARK | 3 | | | | | | | |
| REMARK | 3 | TLS DETAILS | | | | | | |
| REMARK | 3 | NUMBER OF TLS GROUPS : NULL | | | | | | |
| REMARK | 3 | | | | | | | |
| REMARK | 3 | | | | | | | |
| REMARK | 3 | BULK SOLVENT MODELLING. | | | | | | |
| REMARK | 3 | METHOD USED: BABINET MODEL WITH MASK | | | | | | |
| REMARK | 3 | PARAMETERS FOR MASK CALCULATION | | | | | | |
| REMARK | 3 | VDW PROBE RADIUS : 1.40 | | | | | | |
| REMARK | 3 | ION PROBE RADIUS : 0.80 | | | | | | |
| REMARK | 3 | SHRINKAGE RADIUS : 0.80 | | | | | | |
| REMARK | 3 | | | | | | | |
| REMARK | 3 | OTHER REFINEMENT REMARKS: | | | | | | |
| REMARK | 3 | HYDROGENS HAVE BEEN ADDED IN THE RIDING POSITIONS | | | | | | |
| REMARK | 3 | | | | | | | |
| CISPEP | 1 | SER | A | 22 | PRO | A | 23 | 0.00 |
| CISPEP | 2 | ARG | A | 128 | PRO | A | 129 | 0.00 |
| CISPEP | 3 | GLY | A | 372 | PRO | A | 373 | 0.00 |
| SSBOND | 1 | CYS | A | 155 | CYS | A | 359 | |
| SSBOND | 2 | CYS | A | 217 | CYS | A | 382 | |
| SSBOND | 3 | CYS | A | 269 | CYS | A | 319 | |
| CISPEP | 4 | SER | B | 22 | PRO | B | 23 | 0.00 |
| CISPEP | 5 | ARG | B | 128 | PRO | B | 129 | 0.00 |
| CISPEP | 6 | GLY | B | 372 | PRO | B | 373 | 0.00 |
| SSBOND | 4 | CYS | B | 155 | CYS | B | 359 | |
| SSBOND | 5 | CYS | B | 217 | CYS | B | 382 | |
| SSBOND | 6 | CYS | B | 269 | CYS | B | 319 | |
| CISPEP | 7 | SER | C | 22 | PRO | C | 23 | 0.00 |
| CISPEP | 8 | ARG | C | 128 | PRO | C | 129 | 0.00 |
| CISPEP | 9 | GLY | C | 372 | PRO | C | 373 | 0.00 |
| SSBOND | 7 | CYS | C | 155 | CYS | C | 359 | |
| SSBOND | 8 | CYS | C | 217 | CYS | C | 382 | |
| SSBOND | 9 | CYS | C | 269 | CYS | C | 319 | |

CRYST1 236.630 105.020 62.590 90.00 101.32 90.00 C 1 2 1

| | | | | |
|---|---|---|---|---|
| SCALE1 | 0.004226 | 0.000000 | 0.000846 | 0.00000 |
| SCALE2 | 0.000000 | 0.009522 | 0.000000 | 0.00000 |
| SCALE3 | 0.000000 | 0.000000 | 0.016294 | 0.00000 |

| | | | | | | | | | | | |
|---|---|---|---|---|---|---|---|---|---|---|---|
| ATOM | 1 | N | SER | A | −2 | 17.150 | −22.147 | 12.324 | 1.00 | 34.66 | N |
| ATOM | 3 | CA | SER | A | −2 | 18.119 | −22.435 | 11.206 | 1.00 | 39.63 | C |
| ATOM | 5 | CB | SER | A | −2 | 17.523 | −22.274 | 9.764 | 1.00 | 43.29 | C |
| ATOM | 8 | OG | SER | A | −2 | 17.135 | −20.963 | 9.336 | 1.00 | 46.72 | O |
| ATOM | 10 | C | SER | A | −2 | 19.507 | −21.833 | 11.428 | 1.00 | 41.17 | C |
| ATOM | 11 | O | SER | A | −2 | 20.479 | −22.532 | 11.183 | 1.00 | 58.68 | O |
| ATOM | 14 | N | PHE | A | −1 | 19.649 | −20.573 | 11.846 | 1.00 | 44.17 | N |
| ATOM | 16 | CA | PHE | A | −1 | 20.910 | −20.078 | 12.436 | 1.00 | 38.71 | C |
| ATOM | 18 | CB | PHE | A | −1 | 21.526 | −19.134 | 11.408 | 1.00 | 40.94 | C |
| ATOM | 21 | CG | PHE | A | −1 | 21.855 | −19.846 | 10.113 | 1.00 | 34.80 | C |
| ATOM | 22 | CD1 | PHE | A | −1 | 20.860 | −20.099 | 9.181 | 1.00 | 33.03 | C |
| ATOM | 24 | CE1 | PHE | A | −1 | 21.162 | −20.772 | 8.020 | 1.00 | 44.01 | C |
| ATOM | 26 | CZ | PHE | A | −1 | 22.473 | −21.212 | 7.783 | 1.00 | 40.67 | C |
| ATOM | 28 | CE2 | PHE | A | −1 | 23.455 | −20.971 | 8.704 | 1.00 | 41.60 | C |
| ATOM | 30 | CD2 | PHE | A | −1 | 23.146 | −20.297 | 9.864 | 1.00 | 41.30 | C |
| ATOM | 32 | C | PHE | A | −1 | 20.809 | −19.447 | 13.873 | 1.00 | 48.71 | C |
| ATOM | 33 | O | PHE | A | −1 | 21.220 | −18.282 | 14.082 | 1.00 | 52.36 | O |
| ATOM | 34 | N | VAL | A | 0 | 20.309 | −20.241 | 14.848 | 1.00 | 50.55 | N |
| ATOM | 36 | CA | VAL | A | 0 | 19.944 | −19.816 | 16.243 | 1.00 | 50.67 | C |
| ATOM | 38 | CB | VAL | A | 0 | 19.252 | −20.960 | 17.037 | 1.00 | 54.02 | C |
| ATOM | 40 | CG1 | VAL | A | 0 | 17.851 | −21.180 | 16.565 | 1.00 | 59.81 | C |
| ATOM | 44 | CG2 | VAL | A | 0 | 20.016 | −22.211 | 16.926 | 1.00 | 54.45 | C |
| ATOM | 48 | C | VAL | A | 0 | 21.047 | −19.362 | 17.171 | 1.00 | 47.63 | C |
| ATOM | 49 | O | VAL | A | 0 | 20.864 | −18.642 | 18.174 | 1.00 | 47.19 | O |
| ATOM | 50 | N | GLU | A | 1 | 22.195 | −19.867 | 16.855 | 1.00 | 47.53 | N |
| ATOM | 52 | CA | GLU | A | 1 | 23.444 | −19.407 | 17.410 | 1.00 | 45.35 | C |
| ATOM | 54 | CB | GLU | A | 1 | 24.489 | −19.994 | 16.474 | 1.00 | 46.47 | C |
| ATOM | 57 | CG | GLU | A | 1 | 23.893 | −20.218 | 15.070 | 1.00 | 37.41 | C |
| ATOM | 60 | CD | GLU | A | 1 | 24.627 | −21.224 | 14.257 | 1.00 | 36.90 | C |
| ATOM | 61 | OE1 | GLU | A | 1 | 23.964 | −21.926 | 13.487 | 1.00 | 29.13 | O |
| ATOM | 62 | OE2 | GLU | A | 1 | 25.868 | −21.304 | 14.382 | 1.00 | 44.61 | O |
| ATOM | 63 | C | GLU | A | 1 | 23.573 | −17.933 | 17.273 | 1.00 | 44.07 | C |
| ATOM | 64 | O | GLU | A | 1 | 23.950 | −17.227 | 18.185 | 1.00 | 47.47 | O |
| ATOM | 65 | N | MET | A | 2 | 23.211 | −17.471 | 16.087 | 1.00 | 48.56 | N |
| ATOM | 67 | CA | MET | A | 2 | 23.536 | −16.135 | 15.628 | 1.00 | 44.26 | C |
| ATOM | 69 | CB | MET | A | 2 | 23.886 | −16.271 | 14.156 | 1.00 | 46.78 | C |
| ATOM | 72 | CG | MET | A | 2 | 24.968 | −17.287 | 13.979 | 1.00 | 46.07 | C |
| ATOM | 75 | SD | MET | A | 2 | 25.968 | −17.107 | 12.614 | 1.00 | 44.85 | S |
| ATOM | 76 | CE | MET | A | 2 | 25.329 | −18.379 | 11.580 | 1.00 | 47.63 | C |
| ATOM | 80 | C | MET | A | 2 | 22.441 | −15.143 | 15.782 | 1.00 | 44.93 | C |
| ATOM | 81 | O | MET | A | 2 | 22.694 | −13.949 | 15.928 | 1.00 | 44.66 | O |
| ATOM | 82 | N | VAL | A | 3 | 21.213 | −15.640 | 15.739 | 1.00 | 44.93 | N |
| ATOM | 84 | CA | VAL | A | 3 | 20.054 | −14.792 | 15.864 | 1.00 | 42.74 | C |
| ATOM | 86 | CB | VAL | A | 3 | 18.787 | −15.658 | 15.886 | 1.00 | 41.67 | C |
| ATOM | 88 | CG1 | VAL | A | 3 | 17.725 | −15.033 | 16.713 | 1.00 | 37.90 | C |
| ATOM | 92 | CG2 | VAL | A | 3 | 18.319 | −15.879 | 14.506 | 1.00 | 37.99 | C |
| ATOM | 96 | C | VAL | A | 3 | 20.178 | −14.009 | 17.153 | 1.00 | 43.63 | C |
| ATOM | 97 | O | VAL | A | 3 | 20.474 | −14.592 | 18.164 | 1.00 | 38.26 | O |
| ATOM | 98 | N | ASP | A | 4 | 19.953 | −12.696 | 17.100 | 1.00 | 45.42 | N |
| ATOM | 100 | CA | ASP | A | 4 | 20.026 | −11.823 | 18.267 | 1.00 | 45.76 | C |
| ATOM | 102 | CB | ASP | A | 4 | 19.330 | −12.445 | 19.482 | 1.00 | 53.20 | C |
| ATOM | 105 | CG | ASP | A | 4 | 17.803 | −12.638 | 19.299 | 1.00 | 65.48 | C |
| ATOM | 106 | OD1 | ASP | A | 4 | 17.222 | −13.367 | 20.153 | 1.00 | 74.58 | O |
| ATOM | 107 | OD2 | ASP | A | 4 | 17.098 | −12.144 | 18.367 | 1.00 | 62.82 | O |
| ATOM | 108 | C | ASP | A | 4 | 21.417 | −11.521 | 18.742 | 1.00 | 45.27 | C |
| ATOM | 109 | O | ASP | A | 4 | 21.561 | −11.253 | 19.923 | 1.00 | 50.54 | O |
| ATOM | 110 | N | ASN | A | 5 | 22.446 | −11.539 | 17.888 | 1.00 | 41.26 | N |
| ATOM | 112 | CA | ASN | A | 5 | 23.800 | −11.339 | 18.387 | 1.00 | 35.44 | C |
| ATOM | 114 | CB | ASN | A | 5 | 24.717 | −12.331 | 17.728 | 1.00 | 37.18 | C |
| ATOM | 117 | CG | ASN | A | 5 | 24.759 | −12.137 | 16.262 | 1.00 | 39.34 | C |
| ATOM | 118 | OD1 | ASN | A | 5 | 23.857 | −11.544 | 15.736 | 1.00 | 38.77 | O |
| ATOM | 119 | ND2 | ASN | A | 5 | 25.811 | −12.618 | 15.586 | 1.00 | 35.75 | N |
| ATOM | 122 | C | ASN | A | 5 | 24.387 | −9.939 | 18.228 | 1.00 | 38.11 | C |
| ATOM | 123 | O | ASN | A | 5 | 25.585 | −9.733 | 18.468 | 1.00 | 37.36 | O |
| ATOM | 124 | N | LEU | A | 6 | 23.549 | −8.980 | 17.833 | 1.00 | 38.41 | N |
| ATOM | 126 | CA | LEU | A | 6 | 23.939 | −7.572 | 17.679 | 1.00 | 33.19 | C |
| ATOM | 128 | CB | LEU | A | 6 | 23.468 | −7.098 | 16.340 | 1.00 | 26.93 | C |
| ATOM | 131 | CG | LEU | A | 6 | 24.086 | −7.474 | 15.007 | 1.00 | 27.79 | C |
| ATOM | 133 | CD1 | LEU | A | 6 | 23.717 | −6.361 | 14.059 | 1.00 | 27.07 | C |
| ATOM | 137 | CD2 | LEU | A | 6 | 25.567 | −7.678 | 14.998 | 1.00 | 22.82 | C |
| ATOM | 141 | C | LEU | A | 6 | 23.293 | −6.664 | 18.756 | 1.00 | 36.63 | C |
| ATOM | 142 | O | LEU | A | 6 | 22.262 | −7.002 | 19.284 | 1.00 | 46.36 | O |
| ATOM | 143 | N | ARG | A | 7 | 23.875 | −5.513 | 19.075 | 1.00 | 41.26 | N |
| ATOM | 145 | CA | ARG | A | 7 | 23.290 | −4.554 | 20.038 | 1.00 | 40.38 | C |
| ATOM | 147 | CB | ARG | A | 7 | 23.833 | −4.740 | 21.451 | 1.00 | 44.61 | C |
| ATOM | 150 | CG | ARG | A | 7 | 23.137 | −5.889 | 22.209 | 1.00 | 57.60 | C |
| ATOM | 153 | CD | ARG | A | 7 | 23.680 | −6.233 | 23.630 | 1.00 | 65.11 | C |
| ATOM | 156 | NE | ARG | A | 7 | 25.141 | −6.399 | 23.667 | 1.00 | 76.98 | N |
| ATOM | 158 | CZ | ARG | A | 7 | 25.947 | −6.088 | 24.706 | 1.00 | 82.43 | C |
| ATOM | 159 | NH1 | ARG | A | 7 | 25.468 | −5.589 | 25.845 | 1.00 | 83.89 | N |
| ATOM | 162 | NH2 | ARG | A | 7 | 27.256 | −6.284 | 24.603 | 1.00 | 82.28 | N |
| ATOM | 165 | C | ARG | A | 7 | 23.627 | −3.196 | 19.506 | 1.00 | 37.09 | C |
| ATOM | 166 | O | ARG | A | 7 | 24.329 | −3.119 | 18.506 | 1.00 | 39.77 | O |
| ATOM | 167 | N | GLY | A | 8 | 23.139 | −2.136 | 20.139 | 1.00 | 41.94 | N |
| ATOM | 169 | CA | GLY | A | 8 | 23.316 | −0.770 | 19.642 | 1.00 | 46.85 | C |
| ATOM | 172 | C | GLY | A | 8 | 22.093 | −0.084 | 18.994 | 1.00 | 53.78 | C |
| ATOM | 173 | O | GLY | A | 8 | 20.926 | −0.485 | 19.254 | 1.00 | 49.83 | O |
| ATOM | 174 | N | LYS | A | 9 | 22.345 | 0.968 | 18.169 | 1.00 | 56.06 | N |
| ATOM | 176 | CA | LYS | A | 9 | 21.272 | 1.585 | 17.372 | 1.00 | 58.92 | C |
| ATOM | 178 | CB | LYS | A | 9 | 20.312 | 2.324 | 18.314 | 1.00 | 65.60 | C |
| ATOM | 181 | CG | LYS | A | 9 | 20.817 | 3.673 | 18.821 | 1.00 | 71.58 | C |
| ATOM | 184 | CD | LYS | A | 9 | 19.785 | 4.339 | 19.712 | 1.00 | 76.80 | C |
| ATOM | 187 | CE | LYS | A | 9 | 20.460 | 5.364 | 20.645 | 1.00 | 81.30 | C |
| ATOM | 190 | NZ | LYS | A | 9 | 19.689 | 5.694 | 21.898 | 1.00 | 79.59 | N |
| ATOM | 194 | C | LYS | A | 9 | 21.662 | 2.497 | 16.157 | 1.00 | 57.10 | C |
| ATOM | 195 | O | LYS | A | 9 | 22.801 | 2.893 | 15.996 | 1.00 | 53.27 | O |
| ATOM | 196 | N | SER | A | 10 | 20.691 | 2.865 | 15.312 | 1.00 | 58.39 | N |
| ATOM | 198 | CA | SER | A | 10 | 21.022 | 3.699 | 14.153 | 1.00 | 59.37 | C |
| ATOM | 200 | CB | SER | A | 10 | 19.791 | 4.146 | 13.417 | 1.00 | 63.36 | C |
| ATOM | 203 | OG | SER | A | 10 | 18.789 | 4.510 | 14.345 | 1.00 | 73.85 | O |
| ATOM | 205 | C | SER | A | 10 | 21.724 | 4.919 | 14.635 | 1.00 | 61.22 | C |
| ATOM | 206 | O | SER | A | 10 | 22.617 | 5.434 | 13.956 | 1.00 | 64.24 | O |
| ATOM | 207 | N | GLY | A | 11 | 21.279 | 5.370 | 15.818 | 1.00 | 60.57 | N |
| ATOM | 209 | CA | GLY | A | 11 | 21.807 | 6.519 | 16.499 | 1.00 | 49.93 | C |
| ATOM | 212 | C | GLY | A | 11 | 23.308 | 6.455 | 16.511 | 1.00 | 53.46 | C |
| ATOM | 213 | O | GLY | A | 11 | 23.952 | 7.390 | 16.020 | 1.00 | 42.23 | O |
| ATOM | 214 | N | GLN | A | 12 | 23.864 | 5.357 | 17.052 | 1.00 | 51.87 | N |
| ATOM | 216 | CA | GLN | A | 12 | 25.313 | 5.236 | 17.206 | 1.00 | 51.01 | C |
| ATOM | 218 | CB | GLN | A | 12 | 25.656 | 5.405 | 18.653 | 1.00 | 50.53 | C |
| ATOM | 221 | CG | GLN | A | 12 | 25.254 | 6.733 | 19.190 | 1.00 | 53.21 | C |
| ATOM | 224 | CD | GLN | A | 12 | 25.455 | 6.803 | 20.686 | 1.00 | 53.35 | C |
| ATOM | 225 | OE1 | GLN | A | 12 | 25.155 | 5.827 | 21.393 | 1.00 | 51.66 | O |
| ATOM | 226 | NE2 | GLN | A | 12 | 25.960 | 7.941 | 21.174 | 1.00 | 44.80 | N |
| ATOM | 229 | C | GLN | A | 12 | 25.939 | 3.922 | 16.774 | 1.00 | 54.07 | C |
| ATOM | 230 | O | GLN | A | 12 | 27.027 | 3.567 | 17.245 | 1.00 | 62.50 | O |
| ATOM | 231 | N | GLY | A | 13 | 25.264 | 3.200 | 15.897 | 1.00 | 49.53 | N |
| ATOM | 233 | CA | GLY | A | 13 | 25.763 | 1.941 | 15.424 | 1.00 | 43.84 | C |
| ATOM | 236 | C | GLY | A | 13 | 25.250 | 0.639 | 16.051 | 1.00 | 44.95 | C |
| ATOM | 237 | O | GLY | A | 13 | 24.875 | 0.584 | 17.216 | 1.00 | 46.26 | O |
| ATOM | 238 | N | TYR | A | 14 | 25.256 | −0.415 | 15.219 | 1.00 | 37.84 | N |
| ATOM | 240 | CA | TYR | A | 14 | 25.070 | −1.777 | 15.613 | 1.00 | 38.80 | C |
| ATOM | 242 | CB | TYR | A | 14 | 24.228 | −2.432 | 14.566 | 1.00 | 43.50 | C |
| ATOM | 245 | CG | TYR | A | 14 | 22.871 | −1.882 | 14.579 | 1.00 | 40.77 | C |
| ATOM | 246 | CD1 | TYR | A | 14 | 22.592 | −0.703 | 13.951 | 1.00 | 43.94 | C |
| ATOM | 248 | CE1 | TYR | A | 14 | 21.372 | −0.202 | 13.967 | 1.00 | 47.36 | C |
| ATOM | 250 | CZ | TYR | A | 14 | 20.411 | −0.876 | 14.617 | 1.00 | 48.18 | C |
| ATOM | 251 | OH | TYR | A | 14 | 19.178 | −0.405 | 14.638 | 1.00 | 49.40 | O |
| ATOM | 253 | CE2 | TYR | A | 14 | 20.659 | −2.045 | 15.246 | 1.00 | 45.91 | C |
| ATOM | 255 | CD2 | TYR | A | 14 | 21.883 | −2.532 | 15.224 | 1.00 | 39.22 | C |
| ATOM | 257 | C | TYR | A | 14 | 26.388 | −2.532 | 15.658 | 1.00 | 39.26 | C |
| ATOM | 258 | O | TYR | A | 14 | 27.167 | −2.474 | 14.703 | 1.00 | 42.82 | O |
| ATOM | 259 | N | TYR | A | 15 | 26.610 | −3.291 | 16.734 | 1.00 | 38.03 | N |
| ATOM | 261 | CA | TYR | A | 15 | 27.905 | −3.917 | 16.966 | 1.00 | 33.51 | C |
| ATOM | 263 | CB | TYR | A | 15 | 28.655 | −2.978 | 17.885 | 1.00 | 35.56 | C |
| ATOM | 266 | CG | TYR | A | 15 | 28.090 | −2.934 | 19.303 | 1.00 | 38.03 | C |
| ATOM | 267 | CD1 | TYR | A | 15 | 28.139 | −4.052 | 20.116 | 1.00 | 40.86 | C |
| ATOM | 269 | CE1 | TYR | A | 15 | 27.641 | −4.030 | 21.388 | 1.00 | 42.66 | C |
| ATOM | 271 | CZ | TYR | A | 15 | 27.086 | −2.892 | 21.882 | 1.00 | 43.41 | C |
| ATOM | 272 | OH | TYR | A | 15 | 26.593 | −2.912 | 23.176 | 1.00 | 40.43 | O |
| ATOM | 274 | CE2 | TYR | A | 15 | 27.023 | −1.757 | 21.096 | 1.00 | 41.62 | C |
| ATOM | 276 | CD2 | TYR | A | 15 | 27.522 | −1.787 | 19.816 | 1.00 | 38.35 | C |
| ATOM | 278 | C | TYR | A | 15 | 27.828 | −5.307 | 17.605 | 1.00 | 32.70 | C |
| ATOM | 279 | O | TYR | A | 15 | 26.901 | −5.608 | 18.296 | 1.00 | 33.91 | O |
| ATOM | 280 | N | VAL | A | 16 | 28.832 | −6.148 | 17.398 | 1.00 | 38.82 | N |
| ATOM | 282 | CA | VAL | A | 16 | 28.763 | −7.554 | 17.781 | 1.00 | 36.77 | C |
| ATOM | 284 | CB | VAL | A | 16 | 28.826 | −8.444 | 16.556 | 1.00 | 41.62 | C |
| ATOM | 286 | CG1 | VAL | A | 16 | 30.280 | −8.691 | 16.154 | 1.00 | 41.85 | C |
| ATOM | 290 | CG2 | VAL | A | 16 | 28.212 | −9.788 | 16.804 | 1.00 | 46.15 | C |
| ATOM | 294 | C | VAL | A | 16 | 29.993 | −7.861 | 18.579 | 1.00 | 37.30 | C |
| ATOM | 295 | O | VAL | A | 16 | 30.995 | −7.188 | 18.388 | 1.00 | 37.00 | O |
| ATOM | 296 | N | GLU | A | 17 | 29.898 | −8.861 | 19.461 | 1.00 | 32.42 | N |
| ATOM | 298 | CA | GLU | A | 17 | 30.974 | −9.315 | 20.309 | 1.00 | 37.70 | C |
| ATOM | 300 | CB | GLU | A | 17 | 30.339 | −10.181 | 21.407 | 1.00 | 41.03 | C |
| ATOM | 303 | CG | GLU | A | 17 | 31.155 | −10.529 | 22.669 | 1.00 | 41.42 | C |
| ATOM | 306 | CD | GLU | A | 17 | 30.339 | −11.380 | 23.676 | 1.00 | 53.52 | C |
| ATOM | 307 | OE1 | GLU | A | 17 | 29.191 | −10.947 | 24.041 | 1.00 | 62.24 | O |
| ATOM | 308 | OE2 | GLU | A | 17 | 30.807 | −12.484 | 24.114 | 1.00 | 47.86 | O |
| ATOM | 309 | C | GLU | A | 17 | 32.000 | −10.162 | 19.538 | 1.00 | 39.46 | C |
| ATOM | 310 | O | GLU | A | 17 | 31.637 | −11.032 | 18.796 | 1.00 | 39.58 | O |
| ATOM | 311 | N | MET | A | 18 | 33.292 | −9.925 | 19.711 | 1.00 | 44.75 | N |
| ATOM | 313 | CA | MET | A | 18 | 34.301 | −10.791 | 19.082 | 1.00 | 41.54 | C |
| ATOM | 315 | CB | MET | A | 18 | 34.838 | −10.160 | 17.802 | 1.00 | 41.81 | C |
| ATOM | 318 | CG | MET | A | 18 | 33.836 | −9.447 | 16.939 | 1.00 | 41.86 | C |
| ATOM | 321 | SD | MET | A | 18 | 34.600 | −8.833 | 15.425 | 1.00 | 50.01 | S |
| ATOM | 322 | CE | MET | A | 18 | 35.054 | −10.282 | 14.687 | 1.00 | 45.99 | C |
| ATOM | 326 | C | MET | A | 18 | 35.466 | −10.864 | 20.038 | 1.00 | 43.74 | C |
| ATOM | 327 | O | MET | A | 18 | 35.596 | −10.003 | 20.877 | 1.00 | 49.03 | O |
| ATOM | 328 | N | THR | A | 19 | 36.325 | −11.868 | 19.937 | 1.00 | 45.52 | N |
| ATOM | 330 | CA | THR | A | 19 | 37.572 | −11.813 | 20.698 | 1.00 | 45.53 | C |
| ATOM | 332 | CB | THR | A | 19 | 37.747 | −13.023 | 21.572 | 1.00 | 45.33 | C |
| ATOM | 334 | OG1 | THR | A | 19 | 38.226 | −14.108 | 20.759 | 1.00 | 50.86 | O |
| ATOM | 336 | CG2 | THR | A | 19 | 36.463 | −13.451 | 22.142 | 1.00 | 40.03 | C |
| ATOM | 340 | C | THR | A | 19 | 38.763 | −11.893 | 19.814 | 1.00 | 45.19 | C |
| ATOM | 341 | O | THR | A | 19 | 38.693 | −12.560 | 18.785 | 1.00 | 47.81 | O |
| ATOM | 342 | N | VAL | A | 20 | 39.865 | −11.267 | 20.243 | 1.00 | 46.54 | N |
| ATOM | 344 | CA | VAL | A | 20 | 41.181 | −11.385 | 19.556 | 1.00 | 46.08 | C |
| ATOM | 346 | CB | VAL | A | 20 | 41.657 | −10.076 | 19.031 | 1.00 | 44.11 | C |
| ATOM | 348 | CG1 | VAL | A | 20 | 40.734 | −9.589 | 17.998 | 1.00 | 51.93 | C |
| ATOM | 352 | CG2 | VAL | A | 20 | 41.687 | −9.058 | 20.110 | 1.00 | 51.31 | C |
| ATOM | 356 | C | VAL | A | 20 | 42.286 | −11.858 | 20.490 | 1.00 | 48.60 | C |
| ATOM | 357 | O | VAL | A | 20 | 42.242 | −11.643 | 21.715 | 1.00 | 49.22 | O |
| ATOM | 358 | N | GLY | A | 21 | 43.293 | −12.505 | 19.944 | 1.00 | 49.38 | N |
| ATOM | 360 | CA | GLY | A | 21 | 44.406 | −12.881 | 20.791 | 1.00 | 51.59 | C |
| ATOM | 363 | C | GLY | A | 21 | 44.282 | −14.129 | 21.652 | 1.00 | 51.96 | C |
| ATOM | 364 | O | GLY | A | 21 | 43.216 | −14.659 | 21.926 | 1.00 | 48.04 | O |
| ATOM | 365 | N | SER | A | 22 | 45.430 | −14.604 | 22.087 | 1.00 | 51.50 | N |
| ATOM | 367 | CA | SER | A | 22 | 45.462 | −15.754 | 22.916 | 1.00 | 54.86 | C |
| ATOM | 369 | CB | SER | A | 22 | 46.238 | −16.846 | 22.176 | 1.00 | 58.86 | C |
| ATOM | 372 | OG | SER | A | 22 | 45.861 | −16.909 | 20.794 | 1.00 | 60.35 | O |
| ATOM | 374 | C | SER | A | 22 | 46.156 | −15.336 | 24.203 | 1.00 | 54.80 | C |
| ATOM | 375 | O | SER | A | 22 | 47.217 | −14.720 | 24.127 | 1.00 | 53.24 | O |
| ATOM | 376 | N | PRO | A | 23 | 45.551 | −15.566 | 25.378 | 1.00 | 54.99 | N |
| ATOM | 377 | CA | PRO | A | 23 | 44.141 | −15.954 | 25.547 | 1.00 | 56.89 | C |
| ATOM | 379 | CB | PRO | A | 23 | 43.945 | −15.868 | 27.056 | 1.00 | 54.92 | C |
| ATOM | 382 | CG | PRO | A | 23 | 45.075 | −15.007 | 27.510 | 1.00 | 52.28 | C |
| ATOM | 385 | CD | PRO | A | 23 | 46.218 | −15.491 | 26.680 | 1.00 | 53.62 | C |
| ATOM | 388 | C | PRO | A | 23 | 43.244 | −14.889 | 24.943 | 1.00 | 56.89 | C |
| ATOM | 389 | O | PRO | A | 23 | 43.808 | −13.858 | 24.604 | 1.00 | 57.85 | O |
| ATOM | 390 | N | PRO | A | 24 | 41.931 | −15.125 | 24.840 | 1.00 | 56.03 | N |
| ATOM | 391 | CA | PRO | A | 24 | 40.963 | −14.220 | 24.217 | 1.00 | 52.69 | C |
| ATOM | 393 | CB | PRO | A | 24 | 39.667 | −15.023 | 24.276 | 1.00 | 52.70 | C |
| ATOM | 396 | CG | PRO | A | 24 | 40.058 | −16.369 | 24.440 | 1.00 | 55.89 | C |
| ATOM | 399 | CD | PRO | A | 24 | 41.259 | −16.335 | 25.325 | 1.00 | 57.99 | C |
| ATOM | 402 | C | PRO | A | 24 | 40.697 | −12.963 | 24.971 | 1.00 | 52.11 | C |
| ATOM | 403 | O | PRO | A | 24 | 40.598 | −13.028 | 26.170 | 1.00 | 56.31 | O |
| ATOM | 404 | N | GLN | A | 25 | 40.558 | −11.856 | 24.261 | 1.00 | 53.37 | N |
| ATOM | 406 | CA | GLN | A | 25 | 40.215 | −10.568 | 24.839 | 1.00 | 54.86 | C |
| ATOM | 408 | CB | GLN | A | 25 | 41.290 | −9.528 | 24.531 | 1.00 | 55.07 | C |
| ATOM | 411 | CG | GLN | A | 25 | 42.627 | −9.938 | 24.959 | 1.00 | 57.87 | C |
| ATOM | 414 | CD | GLN | A | 25 | 43.630 | −8.867 | 24.743 | 1.00 | 56.36 | C |
| ATOM | 415 | OE1 | GLN | A | 25 | 43.283 | −7.691 | 24.741 | 1.00 | 56.87 | O |
| ATOM | 416 | NE2 | GLN | A | 25 | 44.888 | −9.263 | 24.561 | 1.00 | 48.88 | N |
| ATOM | 419 | C | GLN | A | 25 | 38.946 | −10.098 | 24.168 | 1.00 | 54.86 | C |
| ATOM | 420 | O | GLN | A | 25 | 38.936 | −9.799 | 22.968 | 1.00 | 58.85 | O |
| ATOM | 421 | N | THR | A | 26 | 37.883 | −10.006 | 24.944 | 1.00 | 52.85 | N |
| ATOM | 423 | CA | THR | A | 26 | 36.604 | −9.597 | 24.436 | 1.00 | 50.69 | C |
| ATOM | 425 | CB | THR | A | 26 | 35.614 | −9.763 | 25.531 | 1.00 | 49.93 | C |
| ATOM | 427 | OG1 | THR | A | 26 | 35.404 | −11.164 | 25.799 | 1.00 | 53.75 | O |
| ATOM | 429 | CG2 | THR | A | 26 | 34.308 | −9.246 | 25.060 | 1.00 | 46.30 | C |
| ATOM | 433 | C | THR | A | 26 | 36.575 | −8.146 | 24.003 | 1.00 | 51.52 | C |
| ATOM | 434 | O | THR | A | 26 | 37.078 | −7.284 | 24.696 | 1.00 | 59.94 | O |
| ATOM | 435 | N | LEU | A | 27 | 35.982 | −7.856 | 22.858 | 1.00 | 49.68 | N |
| ATOM | 437 | CA | LEU | A | 27 | 35.858 | −6.476 | 22.412 | 1.00 | 39.53 | C |
| ATOM | 439 | CB | LEU | A | 27 | 37.066 | −6.094 | 21.592 | 1.00 | 42.69 | C |
| ATOM | 442 | CG | LEU | A | 27 | 38.456 | −5.850 | 22.241 | 1.00 | 44.73 | C |
| ATOM | 444 | CD1 | LEU | A | 27 | 39.404 | −5.301 | 21.192 | 1.00 | 42.23 | C |
| ATOM | 448 | CD2 | LEU | A | 27 | 38.418 | −4.865 | 23.382 | 1.00 | 44.45 | C |
| ATOM | 452 | C | LEU | A | 27 | 34.615 | −6.390 | 21.574 | 1.00 | 40.04 | C |
| ATOM | 453 | O | LEU | A | 27 | 34.313 | −7.319 | 20.821 | 1.00 | 40.69 | O |
| ATOM | 454 | N | ASN | A | 28 | 33.859 | −5.305 | 21.704 | 1.00 | 43.27 | N |
| ATOM | 456 | CA | ASN | A | 28 | 32.654 | −5.129 | 20.873 | 1.00 | 43.26 | C |
| ATOM | 458 | CB | ASN | A | 28 | 31.509 | −4.465 | 21.654 | 1.00 | 45.19 | C |
| ATOM | 461 | CG | ASN | A | 28 | 30.830 | −5.417 | 22.672 | 1.00 | 44.78 | C |
| ATOM | 462 | OD1 | ASN | A | 28 | 30.936 | −5.234 | 23.888 | 1.00 | 47.19 | O |
| ATOM | 463 | ND2 | ASN | A | 28 | 30.123 | −6.411 | 22.174 | 1.00 | 42.58 | N |
| ATOM | 466 | C | ASN | A | 28 | 33.018 | −4.308 | 19.633 | 1.00 | 45.28 | C |
| ATOM | 467 | O | ASN | A | 28 | 33.476 | −3.172 | 19.775 | 1.00 | 46.40 | O |
| ATOM | 468 | N | ILE | A | 29 | 32.795 | −4.911 | 18.450 | 1.00 | 46.88 | N |
| ATOM | 470 | CA | ILE | A | 29 | 33.115 | −4.400 | 17.094 | 1.00 | 44.20 | C |
| ATOM | 472 | CB | ILE | A | 29 | 33.690 | −5.561 | 16.303 | 1.00 | 43.47 | C |
| ATOM | 474 | CG1 | ILE | A | 29 | 34.737 | −6.291 | 17.121 | 1.00 | 43.10 | C |
| ATOM | 477 | CD1 | ILE | A | 29 | 35.870 | −5.421 | 17.574 | 1.00 | 44.94 | C |
| ATOM | 481 | CG2 | ILE | A | 29 | 34.248 | −5.079 | 14.973 | 1.00 | 46.96 | C |
| ATOM | 485 | C | ILE | A | 29 | 31.942 | −3.874 | 16.222 | 1.00 | 41.20 | C |
| ATOM | 486 | O | ILE | A | 29 | 30.951 | −4.502 | 16.068 | 1.00 | 36.51 | O |
| ATOM | 487 | N | LEU | A | 30 | 32.084 | −2.715 | 15.617 | 1.00 | 44.61 | N |
| ATOM | 489 | CA | LEU | A | 30 | 31.023 | −2.165 | 14.783 | 1.00 | 41.39 | C |
| ATOM | 491 | CB | LEU | A | 30 | 31.284 | −0.684 | 14.562 | 1.00 | 36.72 | C |
| ATOM | 494 | CG | LEU | A | 30 | 30.022 | 0.080 | 14.326 | 1.00 | 38.55 | C |
| ATOM | 496 | OD1 | LEU | A | 30 | 29.865 | 1.229 | 15.255 | 1.00 | 44.29 | C |
| ATOM | 500 | CD2 | LEU | A | 30 | 30.137 | 0.569 | 12.982 | 1.00 | 43.51 | C |
| ATOM | 504 | C | LEU | A | 30 | 30.885 | −2.911 | 13.455 | 1.00 | 40.85 | C |
| ATOM | 505 | O | LEU | A | 30 | 31.851 | −3.397 | 12.897 | 1.00 | 45.44 | O |
| ATOM | 506 | N | VAL | A | 31 | 29.662 | −2.996 | 12.953 | 1.00 | 43.56 | N |
| ATOM | 508 | CA | VAL | A | 31 | 29.362 | −3.774 | 11.762 | 1.00 | 42.56 | C |
| ATOM | 510 | CB | VAL | A | 31 | 28.116 | −4.562 | 12.003 | 1.00 | 44.28 | C |
| ATOM | 512 | CG1 | VAL | A | 31 | 27.424 | −4.814 | 10.739 | 1.00 | 46.25 | C |
| ATOM | 516 | CG2 | VAL | A | 31 | 28.472 | −5.868 | 12.644 | 1.00 | 48.98 | C |
| ATOM | 520 | C | VAL | A | 31 | 29.137 | −2.803 | 10.621 | 1.00 | 42.04 | C |
| ATOM | 521 | O | VAL | A | 31 | 28.088 | −2.172 | 10.588 | 1.00 | 46.43 | O |
| ATOM | 522 | N | ASP | A | 32 | 30.113 | −2.699 | 9.704 | 1.00 | 38.37 | N |
| ATOM | 524 | CA | ASP | A | 32 | 30.170 | −1.655 | 8.699 | 1.00 | 36.43 | C |
| ATOM | 526 | CB | ASP | A | 32 | 31.409 | −0.852 | 8.922 | 1.00 | 41.59 | C |
| ATOM | 529 | CG | ASP | A | 32 | 31.371 | 0.375 | 8.151 | 1.00 | 46.08 | C |
| ATOM | 530 | OD1 | ASP | A | 32 | 30.241 | 0.877 | 8.185 | 1.00 | 58.39 | O |
| ATOM | 531 | OD2 | ASP | A | 32 | 32.299 | 0.905 | 7.467 | 1.00 | 43.43 | O |
| ATOM | 532 | C | ASP | A | 32 | 30.308 | −2.118 | 7.291 | 1.00 | 36.03 | C |
| ATOM | 533 | O | ASP | A | 32 | 31.379 | −2.416 | 6.890 | 1.00 | 36.16 | O |
| ATOM | 534 | N | THR | A | 33 | 29.238 | −2.174 | 6.520 | 1.00 | 35.12 | N |
| ATOM | 536 | CA | THR | A | 33 | 29.341 | −2.680 | 5.164 | 1.00 | 36.24 | C |
| ATOM | 538 | CB | THR | A | 33 | 27.969 | −3.180 | 4.660 | 1.00 | 40.04 | C |
| ATOM | 540 | OG1 | THR | A | 33 | 26.982 | −2.136 | 4.794 | 1.00 | 44.25 | O |
| ATOM | 542 | CG2 | THR | A | 33 | 27.453 | −4.347 | 5.575 | 1.00 | 40.43 | C |
| ATOM | 546 | C | THR | A | 33 | 29.829 | −1.581 | 4.301 | 1.00 | 33.30 | C |
| ATOM | 547 | O | THR | A | 33 | 29.893 | −1.701 | 3.085 | 1.00 | 35.29 | O |
| ATOM | 548 | N | GLY | A | 34 | 30.177 | −0.493 | 4.946 | 1.00 | 31.75 | N |
| ATOM | 550 | CA | GLY | A | 34 | 30.601 | 0.715 | 4.245 | 1.00 | 36.33 | C |
| ATOM | 553 | C | GLY | A | 34 | 32.077 | 0.844 | 4.160 | 1.00 | 39.91 | C |
| ATOM | 554 | O | GLY | A | 34 | 32.531 | 1.605 | 3.314 | 1.00 | 50.07 | O |
| ATOM | 555 | N | SER | A | 35 | 32.814 | 0.107 | 5.004 | 1.00 | 37.10 | N |
| ATOM | 557 | CA | SER | A | 35 | 34.228 | 0.032 | 4.901 | 1.00 | 34.40 | C |
| ATOM | 559 | CB | SER | A | 35 | 34.823 | 0.796 | 6.021 | 1.00 | 36.88 | C |
| ATOM | 562 | OG | SER | A | 35 | 34.960 | −0.016 | 7.136 | 1.00 | 40.38 | O |
| ATOM | 564 | C | SER | A | 35 | 34.785 | −1.406 | 4.922 | 1.00 | 37.08 | C |
| ATOM | 565 | O | SER | A | 35 | 34.045 | −2.332 | 5.176 | 1.00 | 32.07 | O |
| ATOM | 566 | N | SER | A | 36 | 36.104 | −1.546 | 4.668 | 1.00 | 37.61 | N |
| ATOM | 568 | CA | SER | A | 36 | 36.836 | −2.824 | 4.521 | 1.00 | 38.67 | C |
| ATOM | 570 | CB | SER | A | 36 | 37.384 | −2.903 | 3.110 | 1.00 | 39.52 | C |
| ATOM | 573 | OG | SER | A | 36 | 36.354 | −2.718 | 2.156 | 1.00 | 47.74 | O |
| ATOM | 575 | C | SER | A | 36 | 38.079 | −3.054 | 5.406 | 1.00 | 41.95 | C |
| ATOM | 576 | O | SER | A | 36 | 38.978 | −3.850 | 5.074 | 1.00 | 42.44 | O |
| ATOM | 577 | N | ASN | A | 37 | 38.162 | −2.374 | 6.526 | 1.00 | 35.87 | N |
| ATOM | 579 | CA | ASN | A | 37 | 39.299 | −2.563 | 7.336 | 1.00 | 30.94 | C |
| ATOM | 581 | CB | ASN | A | 37 | 39.969 | −1.216 | 7.505 | 1.00 | 36.25 | C |
| ATOM | 584 | CG | ASN | A | 37 | 41.020 | −0.997 | 6.507 | 1.00 | 35.00 | C |
| ATOM | 585 | OD1 | ASN | A | 37 | 40.726 | −0.519 | 5.452 | 1.00 | 41.51 | O |
| ATOM | 586 | ND2 | ASN | A | 37 | 42.275 | −1.379 | 6.819 | 1.00 | 35.89 | N |
| ATOM | 589 | C | ASN | A | 37 | 38.876 | −3.085 | 8.679 | 1.00 | 30.50 | C |
| ATOM | 590 | O | ASN | A | 37 | 37.991 | −2.530 | 9.247 | 1.00 | 34.21 | O |
| ATOM | 591 | N | PHE | A | 38 | 39.478 | −4.157 | 9.184 | 1.00 | 36.14 | N |
| ATOM | 593 | CA | PHE | A | 38 | 39.180 | −4.625 | 10.545 | 1.00 | 36.42 | C |
| ATOM | 595 | CB | PHE | A | 38 | 39.256 | −6.146 | 10.663 | 1.00 | 33.72 | C |
| ATOM | 598 | CG | PHE | A | 38 | 38.886 | −6.678 | 12.040 | 1.00 | 43.00 | C |
| ATOM | 599 | CD1 | PHE | A | 38 | 39.713 | −7.566 | 12.705 | 1.00 | 42.48 | C |
| ATOM | 601 | CE1 | PHE | A | 38 | 39.386 | −8.040 | 13.962 | 1.00 | 47.58 | C |
| ATOM | 603 | CZ | PHE | A | 38 | 38.233 | −7.661 | 14.586 | 1.00 | 42.81 | C |
| ATOM | 605 | CE2 | PHE | A | 38 | 37.394 | −6.785 | 13.954 | 1.00 | 49.46 | C |
| ATOM | 607 | CD2 | PHE | A | 38 | 37.717 | −6.290 | 12.670 | 1.00 | 44.85 | C |
| ATOM | 609 | C | PHE | A | 38 | 40.227 | −3.888 | 11.416 | 1.00 | 40.18 | C |
| ATOM | 610 | O | PHE | A | 38 | 41.406 | −3.760 | 11.017 | 1.00 | 39.66 | O |
| ATOM | 611 | N | ALA | A | 39 | 39.802 | −3.389 | 12.576 | 1.00 | 40.32 | N |
| ATOM | 613 | CA | ALA | A | 39 | 40.656 | −2.515 | 13.403 | 1.00 | 40.28 | C |
| ATOM | 615 | CB | ALA | A | 39 | 40.810 | −1.164 | 12.739 | 1.00 | 40.18 | C |
| ATOM | 619 | C | ALA | A | 39 | 40.020 | −2.343 | 14.757 | 1.00 | 37.73 | C |
| ATOM | 620 | O | ALA | A | 39 | 38.787 | −2.412 | 14.885 | 1.00 | 40.37 | O |
| ATOM | 621 | N | VAL | A | 40 | 40.839 | −2.097 | 15.764 | 1.00 | 38.68 | N |
| ATOM | 623 | CA | VAL | A | 40 | 40.334 | −2.088 | 17.117 | 1.00 | 39.39 | C |
| ATOM | 625 | CB | VAL | A | 40 | 40.369 | −3.507 | 17.636 | 1.00 | 41.17 | C |
| ATOM | 627 | CG1 | VAL | A | 40 | 39.731 | −4.442 | 16.605 | 1.00 | 45.27 | C |
| ATOM | 631 | CG2 | VAL | A | 40 | 41.722 | −4.000 | 17.829 | 1.00 | 46.47 | C |
| ATOM | 635 | C | VAL | A | 40 | 41.080 | −1.078 | 18.005 | 1.00 | 48.51 | C |
| ATOM | 636 | O | VAL | A | 40 | 42.175 | −0.583 | 17.642 | 1.00 | 50.57 | O |
| ATOM | 637 | N | GLY | A | 41 | 40.477 | −0.729 | 19.152 | 1.00 | 47.86 | N |
| ATOM | 639 | CA | GLY | A | 41 | 41.073 | 0.262 | 20.014 | 1.00 | 40.52 | C |
| ATOM | 642 | C | GLY | A | 41 | 42.290 | −0.406 | 20.595 | 1.00 | 44.67 | C |
| ATOM | 643 | O | GLY | A | 41 | 42.140 | −1.387 | 21.332 | 1.00 | 42.27 | O |
| ATOM | 644 | N | ALA | A | 42 | 43.500 | 0.079 | 20.274 | 1.00 | 46.72 | N |
| ATOM | 646 | CA | ALA | A | 42 | 44.724 | −0.467 | 20.914 | 1.00 | 42.69 | C |
| ATOM | 648 | CB | ALA | A | 42 | 45.655 | −0.928 | 19.886 | 1.00 | 42.07 | C |
| ATOM | 652 | C | ALA | A | 42 | 45.444 | 0.477 | 21.883 | 1.00 | 46.86 | C |
| ATOM | 653 | O | ALA | A | 42 | 46.687 | 0.515 | 21.984 | 1.00 | 45.43 | O |
| ATOM | 654 | N | ALA | A | 43 | 44.677 | 1.246 | 22.636 | 1.00 | 50.46 | N |
| ATOM | 656 | CA | ALA | A | 43 | 45.278 | 2.216 | 23.548 | 1.00 | 52.82 | C |
| ATOM | 658 | CB | ALA | A | 43 | 46.320 | 3.073 | 22.854 | 1.00 | 48.10 | C |
| ATOM | 662 | C | ALA | A | 43 | 44.196 | 3.101 | 24.052 | 1.00 | 56.62 | C |
| ATOM | 663 | O | ALA | A | 43 | 43.186 | 3.315 | 23.380 | 1.00 | 57.66 | O |
| ATOM | 664 | N | PRO | A | 44 | 44.446 | 3.628 | 25.238 | 1.00 | 62.73 | N |
| ATOM | 665 | CA | PRO | A | 44 | 43.510 | 4.492 | 25.962 | 1.00 | 59.53 | C |
| ATOM | 667 | CB | PRO | A | 44 | 44.390 | 5.093 | 27.053 | 1.00 | 62.72 | C |
| ATOM | 670 | CG | PRO | A | 44 | 45.835 | 4.754 | 26.603 | 1.00 | 66.12 | C |
| ATOM | 673 | CD | PRO | A | 44 | 45.696 | 3.411 | 26.003 | 1.00 | 65.12 | C |
| ATOM | 676 | C | PRO | A | 44 | 42.905 | 5.606 | 25.132 | 1.00 | 59.62 | C |
| ATOM | 677 | O | PRO | A | 44 | 43.553 | 6.133 | 24.218 | 1.00 | 55.79 | O |
| ATOM | 678 | N | HIS | A | 45 | 41.653 | 5.939 | 25.452 | 1.00 | 54.87 | N |
| ATOM | 680 | CA | HIS | A | 45 | 40.970 | 7.043 | 24.813 | 1.00 | 54.75 | C |
| ATOM | 682 | CB | HIS | A | 45 | 40.782 | 6.831 | 23.332 | 1.00 | 50.69 | C |
| ATOM | 685 | CG | HIS | A | 45 | 39.905 | 7.860 | 22.704 | 1.00 | 47.13 | C |
| ATOM | 686 | ND1 | HIS | A | 45 | 38.634 | 8.126 | 23.167 | 1.00 | 46.73 | N |
| ATOM | 688 | CE1 | HIS | A | 45 | 38.094 | 9.082 | 22.429 | 1.00 | 49.26 | C |
| ATOM | 690 | NE2 | HIS | A | 45 | 38.965 | 9.443 | 21.501 | 1.00 | 40.27 | N |
| ATOM | 692 | CD2 | HIS | A | 45 | 40.108 | 8.692 | 21.654 | 1.00 | 46.25 | C |
| ATOM | 694 | C | HIS | A | 45 | 39.645 | 7.185 | 25.525 | 1.00 | 61.01 | C |
| ATOM | 695 | O | HIS | A | 45 | 38.869 | 6.236 | 25.629 | 1.00 | 68.54 | O |
| ATOM | 696 | N | PRO | A | 46 | 39.368 | 8.398 | 25.976 | 1.00 | 66.55 | N |
| ATOM | 697 | CA | PRO | A | 46 | 38.316 | 8.630 | 26.973 | 1.00 | 67.17 | C |
| ATOM | 699 | CB | PRO | A | 46 | 37.953 | 10.103 | 26.772 | 1.00 | 67.46 | C |
| ATOM | 702 | CG | PRO | A | 46 | 39.078 | 10.729 | 25.902 | 1.00 | 64.56 | C |
| ATOM | 705 | CD | PRO | A | 46 | 39.993 | 9.648 | 25.496 | 1.00 | 65.48 | C |
| ATOM | 708 | C | PRO | A | 46 | 37.104 | 7.770 | 26.736 | 1.00 | 65.38 | C |
| ATOM | 709 | O | PRO | A | 46 | 36.433 | 7.311 | 27.650 | 1.00 | 68.92 | O |
| ATOM | 710 | N | PHE | A | 47 | 36.823 | 7.530 | 25.479 | 1.00 | 62.71 | N |
| ATOM | 712 | CA | PHE | A | 47 | 35.613 | 6.833 | 25.156 | 1.00 | 60.29 | C |
| ATOM | 714 | CB | PHE | A | 47 | 35.137 | 7.330 | 23.806 | 1.00 | 58.61 | C |
| ATOM | 717 | CG | PHE | A | 47 | 34.910 | 8.805 | 23.780 | 1.00 | 57.85 | C |
| ATOM | 718 | CD1 | PHE | A | 47 | 34.809 | 9.490 | 22.583 | 1.00 | 54.09 | C |
| ATOM | 720 | CE1 | PHE | A | 47 | 34.602 | 10.831 | 22.559 | 1.00 | 50.97 | C |
| ATOM | 722 | CZ | PHE | A | 47 | 34.486 | 11.529 | 23.739 | 1.00 | 57.26 | C |
| ATOM | 724 | CE2 | PHE | A | 47 | 34.583 | 10.865 | 24.944 | 1.00 | 60.84 | C |
| ATOM | 726 | CD2 | PHE | A | 47 | 34.792 | 9.507 | 24.965 | 1.00 | 59.30 | C |
| ATOM | 728 | C | PHE | A | 47 | 35.728 | 5.340 | 25.155 | 1.00 | 58.83 | C |
| ATOM | 729 | O | PHE | A | 47 | 34.806 | 4.684 | 24.721 | 1.00 | 61.67 | O |
| ATOM | 730 | N | LEU | A | 48 | 36.829 | 4.774 | 25.625 | 1.00 | 58.46 | N |
| ATOM | 732 | CA | LEU | A | 48 | 36.896 | 3.322 | 25.635 | 1.00 | 61.24 | C |
| ATOM | 734 | CB | LEU | A | 48 | 38.084 | 2.812 | 24.786 | 1.00 | 59.23 | C |
| ATOM | 737 | CG | LEU | A | 48 | 38.037 | 3.121 | 23.271 | 1.00 | 60.91 | C |
| ATOM | 739 | CD1 | LEU | A | 48 | 39.439 | 3.297 | 22.652 | 1.00 | 56.82 | C |
| ATOM | 743 | CD2 | LEU | A | 48 | 37.235 | 2.074 | 22.456 | 1.00 | 61.09 | C |
| ATOM | 747 | C | LEU | A | 48 | 36.928 | 2.709 | 27.037 | 1.00 | 62.47 | C |
| ATOM | 748 | O | LEU | A | 48 | 37.767 | 3.065 | 27.846 | 1.00 | 61.13 | O |
| ATOM | 749 | N | HIS | A | 49 | 35.984 | 1.803 | 27.305 | 1.00 | 65.56 | N |
| ATOM | 751 | CA | HIS | A | 49 | 36.036 | 0.929 | 28.489 | 1.00 | 66.38 | C |
| ATOM | 753 | CB | HIS | A | 49 | 34.791 | 0.037 | 28.642 | 1.00 | 73.64 | C |
| ATOM | 756 | CG | HIS | A | 49 | 33.560 | 0.785 | 29.012 | 1.00 | 78.39 | C |
| ATOM | 757 | ND1 | HIS | A | 49 | 33.631 | 2.037 | 29.570 | 1.00 | 87.79 | N |
| ATOM | 759 | CE1 | HIS | A | 49 | 32.415 | 2.503 | 29.791 | 1.00 | 85.97 | C |
| ATOM | 761 | NE2 | HIS | A | 49 | 31.550 | 1.587 | 29.399 | 1.00 | 84.81 | N |
| ATOM | 763 | CD2 | HIS | A | 49 | 32.241 | 0.494 | 28.908 | 1.00 | 79.17 | C |
| ATOM | 765 | C | HIS | A | 49 | 37.173 | −0.070 | 28.366 | 1.00 | 60.95 | C |
| ATOM | 766 | O | HIS | A | 49 | 37.563 | −0.684 | 29.362 | 1.00 | 60.23 | O |
| ATOM | 767 | N | ARG | A | 50 | 37.669 | −0.304 | 27.149 | 1.00 | 58.11 | N |
| ATOM | 769 | CA | ARG | A | 50 | 38.768 | −1.253 | 26.923 | 1.00 | 55.40 | C |
| ATOM | 771 | CB | ARG | A | 50 | 38.247 | −2.671 | 26.985 | 1.00 | 55.21 | C |
| ATOM | 774 | CG | ARG | A | 50 | 36.757 | −2.807 | 26.821 | 1.00 | 54.36 | C |
| ATOM | 777 | CD | ARG | A | 50 | 36.239 | −4.211 | 27.167 | 1.00 | 48.77 | C |
| ATOM | 780 | NE | ARG | A | 50 | 35.083 | −4.540 | 26.350 | 1.00 | 52.02 | N |
| ATOM | 782 | CZ | ARG | A | 50 | 34.594 | −5.760 | 26.210 | 1.00 | 49.86 | C |
| ATOM | 783 | NH1 | ARG | A | 50 | 35.170 | −6.765 | 26.847 | 1.00 | 49.97 | N |
| ATOM | 786 | NH2 | ARG | A | 50 | 33.529 | −5.971 | 25.440 | 1.00 | 44.75 | N |
| ATOM | 789 | C | ARG | A | 50 | 39.577 | −1.078 | 25.631 | 1.00 | 56.08 | C |
| ATOM | 790 | O | ARG | A | 50 | 39.388 | −0.103 | 24.861 | 1.00 | 55.23 | O |
| ATOM | 791 | N | TYR | A | 51 | 40.506 | −2.013 | 25.413 | 1.00 | 56.91 | N |
| ATOM | 793 | CA | TYR | A | 51 | 41.319 | −2.010 | 24.195 | 1.00 | 57.53 | C |
| ATOM | 795 | CB | TYR | A | 51 | 42.153 | −0.735 | 24.085 | 1.00 | 58.76 | C |
| ATOM | 798 | CG | TYR | A | 51 | 43.220 | −0.483 | 25.148 | 1.00 | 65.10 | C |
| ATOM | 799 | CD1 | TYR | A | 51 | 44.487 | −1.041 | 25.057 | 1.00 | 70.53 | C |
| ATOM | 801 | CE1 | TYR | A | 51 | 45.460 | −0.792 | 26.036 | 1.00 | 68.47 | C |
| ATOM | 803 | CZ | TYR | A | 51 | 45.171 | 0.023 | 27.101 | 1.00 | 64.05 | C |
| ATOM | 804 | OH | TYR | A | 51 | 46.091 | 0.286 | 28.077 | 1.00 | 56.09 | O |
| ATOM | 806 | CE2 | TYR | A | 51 | 43.947 | 0.573 | 27.198 | 1.00 | 65.65 | C |
| ATOM | 808 | CD2 | TYR | A | 51 | 42.971 | 0.328 | 26.222 | 1.00 | 64.80 | C |
| ATOM | 810 | C | TYR | A | 51 | 42.211 | −3.222 | 24.026 | 1.00 | 56.08 | C |
| ATOM | 811 | O | TYR | A | 51 | 42.650 | −3.810 | 24.983 | 1.00 | 63.38 | O |
| ATOM | 812 | N | TYR | A | 52 | 42.456 | −3.600 | 22.783 | 1.00 | 56.18 | N |
| ATOM | 814 | CA | TYR | A | 52 | 43.369 | −4.698 | 22.440 | 1.00 | 54.65 | C |
| ATOM | 816 | CB | TYR | A | 52 | 43.609 | −4.634 | 20.954 | 1.00 | 52.23 | C |
| ATOM | 819 | CG | TYR | A | 52 | 44.309 | −5.802 | 20.342 | 1.00 | 49.44 | C |
| ATOM | 820 | CD1 | TYR | A | 52 | 44.364 | −7.038 | 20.958 | 1.00 | 49.18 | C |
| ATOM | 822 | CE1 | TYR | A | 52 | 45.014 | −8.084 | 20.352 | 1.00 | 44.95 | C |
| ATOM | 824 | CZ | TYR | A | 52 | 45.598 | −7.880 | 19.123 | 1.00 | 40.40 | C |
| ATOM | 825 | OH | TYR | A | 52 | 46.257 | −8.880 | 18.453 | 1.00 | 43.51 | O |
| ATOM | 827 | CE2 | TYR | A | 52 | 45.543 | −6.683 | 18.523 | 1.00 | 36.90 | C |
| ATOM | 829 | CD2 | TYR | A | 52 | 44.906 | −5.663 | 19.117 | 1.00 | 44.53 | C |
| ATOM | 831 | C | TYR | A | 52 | 44.716 | −4.512 | 23.090 | 1.00 | 56.76 | C |
| ATOM | 832 | O | TYR | A | 52 | 45.267 | −3.421 | 23.042 | 1.00 | 61.43 | O |
| ATOM | 833 | N | GLN | A | 53 | 45.269 | −5.562 | 23.694 | 1.00 | 58.49 | N |
| ATOM | 835 | CA | GLN | A | 53 | 46.570 | −5.444 | 24.371 | 1.00 | 50.39 | C |
| ATOM | 837 | CB | GLN | A | 53 | 46.443 | −5.654 | 25.885 | 1.00 | 52.39 | C |
| ATOM | 840 | CG | GLN | A | 53 | 45.728 | −4.466 | 26.574 | 1.00 | 57.22 | C |
| ATOM | 843 | CD | GLN | A | 53 | 45.807 | −4.475 | 28.089 | 1.00 | 59.21 | C |
| ATOM | 844 | OE1 | GLN | A | 53 | 44.778 | −4.523 | 28.763 | 1.00 | 68.21 | O |
| ATOM | 845 | NE2 | GLN | A | 53 | 47.019 | −4.413 | 28.627 | 1.00 | 60.90 | N |
| ATOM | 848 | C | GLN | A | 53 | 47.498 | −6.427 | 23.723 | 1.00 | 48.79 | C |
| ATOM | 849 | O | GLN | A | 53 | 47.660 | −7.571 | 24.144 | 1.00 | 48.67 | O |
| ATOM | 850 | N | ARG | A | 54 | 48.115 | −5.964 | 22.659 | 1.00 | 44.44 | N |
| ATOM | 852 | CA | ARG | A | 54 | 48.994 | −6.809 | 21.908 | 1.00 | 42.47 | C |
| ATOM | 854 | CB | ARG | A | 54 | 49.669 | −5.949 | 20.864 | 1.00 | 42.10 | C |
| ATOM | 857 | CG | ARG | A | 54 | 48.733 | −5.430 | 19.740 | 1.00 | 35.73 | C |
| ATOM | 860 | CD | ARG | A | 54 | 49.465 | −4.449 | 18.787 | 1.00 | 38.01 | C |
| ATOM | 863 | NE | ARG | A | 54 | 49.506 | −3.127 | 19.393 | 1.00 | 46.13 | N |
| ATOM | 865 | CZ | ARG | A | 54 | 50.064 | −2.072 | 18.861 | 1.00 | 54.49 | C |
| ATOM | 866 | NH1 | ARG | A | 54 | 50.646 | −2.176 | 17.682 | 1.00 | 63.14 | N |
| ATOM | 869 | NH2 | ARG | A | 54 | 50.052 | −0.903 | 19.504 | 1.00 | 58.58 | N |
| ATOM | 872 | C | ARG | A | 54 | 50.023 | −7.562 | 22.802 | 1.00 | 46.11 | C |
| ATOM | 873 | O | ARG | A | 54 | 50.297 | −8.750 | 22.623 | 1.00 | 50.14 | O |
| ATOM | 874 | N | GLN | A | 55 | 50.583 | −6.887 | 23.777 | 1.00 | 49.49 | N |
| ATOM | 876 | CA | GLN | A | 55 | 51.570 | −7.530 | 24.638 | 1.00 | 54.83 | C |
| ATOM | 878 | CB | GLN | A | 55 | 52.120 | −6.504 | 25.638 | 1.00 | 56.89 | C |
| ATOM | 881 | CG | GLN | A | 55 | 51.331 | −5.158 | 25.639 | 1.00 | 63.64 | C |
| ATOM | 884 | CD | GLN | A | 55 | 50.389 | −4.991 | 26.815 | 1.00 | 69.23 | C |
| ATOM | 885 | OE1 | GLN | A | 55 | 49.575 | −4.052 | 26.852 | 1.00 | 65.62 | O |
| ATOM | 886 | NE2 | GLN | A | 55 | 50.502 | −5.892 | 27.790 | 1.00 | 75.17 | N |
| ATOM | 889 | C | GLN | A | 55 | 51.054 | −8.780 | 25.379 | 1.00 | 56.17 | C |
| ATOM | 890 | O | GLN | A | 55 | 51.849 | −9.509 | 25.952 | 1.00 | 55.62 | O |
| ATOM | 891 | N | LEU | A | 56 | 49.750 | −9.047 | 25.388 | 1.00 | 55.53 | N |
| ATOM | 893 | CA | LEU | A | 56 | 49.250 | −10.216 | 26.133 | 1.00 | 56.08 | C |
| ATOM | 895 | CB | LEU | A | 56 | 48.009 | −9.835 | 26.904 | 1.00 | 57.00 | C |
| ATOM | 898 | CG | LEU | A | 56 | 48.155 | −8.608 | 27.785 | 1.00 | 61.56 | C |
| ATOM | 900 | CD1 | LEU | A | 56 | 46.968 | −8.502 | 28.711 | 1.00 | 62.62 | C |
| ATOM | 904 | CD2 | LEU | A | 56 | 49.453 | −8.699 | 28.580 | 1.00 | 63.10 | C |
| ATOM | 908 | C | LEU | A | 56 | 48.874 | −11.462 | 25.352 | 1.00 | 56.64 | C |
| ATOM | 909 | O | LEU | A | 56 | 48.488 | −12.472 | 25.953 | 1.00 | 56.22 | O |
| ATOM | 910 | N | SER | A | 57 | 48.965 | −11.396 | 24.029 | 1.00 | 55.33 | N |
| ATOM | 912 | CA | SER | A | 57 | 48.533 | −12.496 | 23.198 | 1.00 | 51.40 | C |
| ATOM | 914 | CB | SER | A | 57 | 47.674 | −12.011 | 22.060 | 1.00 | 50.76 | C |
| ATOM | 917 | OG | SER | A | 57 | 47.423 | −13.069 | 21.154 | 1.00 | 51.82 | O |
| ATOM | 919 | C | SER | A | 57 | 49.768 | −13.087 | 22.633 | 1.00 | 54.74 | C |
| ATOM | 920 | O | SER | A | 57 | 50.673 | −12.386 | 22.229 | 1.00 | 64.50 | O |
| ATOM | 921 | N | SER | A | 58 | 49.827 | −14.384 | 22.582 | 1.00 | 51.36 | N |
| ATOM | 923 | CA | SER | A | 58 | 51.036 | −14.980 | 22.152 | 1.00 | 52.68 | C |
| ATOM | 925 | CB | SER | A | 58 | 51.243 | −16.234 | 22.948 | 1.00 | 55.44 | C |
| ATOM | 928 | OG | SER | A | 58 | 50.240 | −17.142 | 22.588 | 1.00 | 51.95 | O |
| ATOM | 930 | C | SER | A | 58 | 50.913 | −15.342 | 20.717 | 1.00 | 51.45 | C |
| ATOM | 931 | O | SER | A | 58 | 51.810 | −15.932 | 20.154 | 1.00 | 58.22 | O |
| ATOM | 932 | N | THR | A | 59 | 49.790 | −15.005 | 20.122 | 1.00 | 49.52 | N |
| ATOM | 934 | CA | THR | A | 59 | 49.551 | −15.299 | 18.719 | 1.00 | 45.44 | C |
| ATOM | 936 | CB | THR | A | 59 | 48.187 | −15.986 | 18.586 | 1.00 | 43.26 | C |
| ATOM | 938 | OG1 | THR | A | 59 | 47.220 | −15.363 | 19.446 | 1.00 | 40.46 | O |
| ATOM | 940 | CG2 | THR | A | 59 | 48.237 | −17.366 | 19.192 | 1.00 | 43.84 | C |
| ATOM | 944 | C | THR | A | 59 | 49.674 | −13.990 | 17.894 | 1.00 | 48.17 | C |
| ATOM | 945 | O | THR | A | 59 | 49.471 | −13.981 | 16.676 | 1.00 | 49.26 | O |
| ATOM | 946 | N | TYR | A | 60 | 50.023 | −12.884 | 18.556 | 1.00 | 44.42 | N |
| ATOM | 948 | CA | TYR | A | 60 | 50.180 | −11.625 | 17.852 | 1.00 | 45.38 | C |
| ATOM | 950 | CB | TYR | A | 60 | 50.484 | −10.499 | 18.843 | 1.00 | 46.42 | C |
| ATOM | 953 | CG | TYR | A | 60 | 50.852 | −9.182 | 18.198 | 1.00 | 46.69 | C |
| ATOM | 954 | CD1 | TYR | A | 60 | 49.945 | −8.500 | 17.379 | 1.00 | 47.22 | C |
| ATOM | 956 | CE1 | TYR | A | 60 | 50.291 | −7.282 | 16.787 | 1.00 | 47.17 | C |
| ATOM | 958 | CZ | TYR | A | 60 | 51.546 | −6.741 | 17.003 | 1.00 | 44.65 | C |
| ATOM | 959 | OH | TYR | A | 60 | 51.894 | −5.538 | 16.408 | 1.00 | 49.47 | O |
| ATOM | 961 | CE2 | TYR | A | 60 | 52.448 | −7.393 | 17.813 | 1.00 | 41.96 | C |
| ATOM | 963 | CD2 | TYR | A | 60 | 52.101 | −8.604 | 18.408 | 1.00 | 45.55 | C |
| ATOM | 965 | C | TYR | A | 60 | 51.325 | −11.709 | 16.878 | 1.00 | 47.40 | C |
| ATOM | 966 | O | TYR | A | 60 | 52.262 | −12.460 | 17.080 | 1.00 | 51.21 | O |
| ATOM | 967 | N | ARG | A | 61 | 51.249 | −10.942 | 15.804 | 1.00 | 48.35 | N |
| ATOM | 969 | CA | ARG | A | 61 | 52.388 | −10.781 | 14.920 | 1.00 | 41.23 | C |
| ATOM | 971 | CB | ARG | A | 61 | 52.392 | −11.782 | 13.795 | 1.00 | 44.25 | C |
| ATOM | 974 | CG | ARG | A | 61 | 52.548 | −13.205 | 14.213 | 1.00 | 45.20 | C |
| ATOM | 977 | CD | ARG | A | 61 | 52.775 | −14.118 | 13.033 | 1.00 | 46.69 | C |
| ATOM | 980 | NE | ARG | A | 61 | 53.343 | −15.391 | 13.450 | 1.00 | 51.89 | N |
| ATOM | 982 | CZ | ARG | A | 61 | 52.893 | −16.545 | 13.066 | 1.00 | 51.83 | C |
| ATOM | 983 | NH1 | ARG | A | 61 | 51.872 | −16.575 | 12.248 | 1.00 | 59.34 | N |
| ATOM | 986 | NH2 | ARG | A | 61 | 53.448 | −17.668 | 13.485 | 1.00 | 50.67 | N |
| ATOM | 989 | C | ARG | A | 61 | 52.287 | −9.433 | 14.302 | 1.00 | 41.40 | C |
| ATOM | 990 | O | ARG | A | 61 | 51.278 | −9.127 | 13.687 | 1.00 | 38.10 | O |
| ATOM | 991 | N | ASP | A | 62 | 53.374 | −8.681 | 14.477 | 1.00 | 45.24 | N |
| ATOM | 993 | CA | ASP | A | 62 | 53.654 | −7.322 | 13.990 | 1.00 | 43.46 | C |
| ATOM | 995 | CB | ASP | A | 62 | 54.947 | −6.899 | 14.658 | 1.00 | 46.00 | C |
| ATOM | 998 | CG | ASP | A | 62 | 55.158 | −5.405 | 14.673 | 1.00 | 50.50 | C |
| ATOM | 999 | OD1 | ASP | A | 62 | 54.562 | −4.701 | 13.833 | 1.00 | 47.18 | O |
| ATOM | 1000 | OD2 | ASP | A | 62 | 55.940 | −4.872 | 15.520 | 1.00 | 56.56 | O |
| ATOM | 1001 | C | ASP | A | 62 | 53.950 | −7.230 | 12.530 | 1.00 | 38.57 | C |
| ATOM | 1002 | O | ASP | A | 62 | 54.868 | −7.840 | 12.078 | 1.00 | 34.49 | O |
| ATOM | 1003 | N | LEU | A | 63 | 53.204 | −6.429 | 11.784 | 1.00 | 42.04 | N |
| ATOM | 1005 | CA | LEU | A | 63 | 53.464 | −6.332 | 10.355 | 1.00 | 41.63 | C |
| ATOM | 1007 | CB | LEU | A | 63 | 52.131 | −6.216 | 9.586 | 1.00 | 39.89 | C |
| ATOM | 1010 | CG | LEU | A | 63 | 51.262 | −7.490 | 9.430 | 1.00 | 39.13 | C |
| ATOM | 1012 | CD1 | LEU | A | 63 | 49.768 | −7.233 | 9.242 | 1.00 | 38.98 | C |
| ATOM | 1016 | CD2 | LEU | A | 63 | 51.755 | −8.283 | 8.272 | 1.00 | 35.93 | C |
| ATOM | 1020 | C | LEU | A | 63 | 54.414 | −5.180 | 10.024 | 1.00 | 45.54 | C |
| ATOM | 1021 | O | LEU | A | 63 | 54.659 | −4.921 | 8.853 | 1.00 | 45.94 | O |
| ATOM | 1022 | N | ARG | A | 64 | 54.933 | −4.467 | 11.034 | 1.00 | 47.05 | N |
| ATOM | 1024 | CA | ARG | A | 64 | 55.873 | −3.385 | 10.779 | 1.00 | 45.84 | C |
| ATOM | 1026 | CB | ARG | A | 64 | 57.261 | −3.976 | 10.447 | 1.00 | 46.95 | C |
| ATOM | 1029 | CG | ARG | A | 64 | 57.974 | −4.717 | 11.615 | 1.00 | 48.68 | C |
| ATOM | 1032 | CD | ARG | A | 64 | 58.685 | −6.010 | 11.233 | 1.00 | 49.84 | C |
| ATOM | 1035 | NE | ARG | A | 64 | 59.846 | −5.810 | 10.371 | 1.00 | 52.18 | N |
| ATOM | 1037 | CZ | ARG | A | 64 | 60.138 | −6.547 | 9.301 | 1.00 | 58.00 | C |
| ATOM | 1038 | NH1 | ARG | A | 64 | 59.360 | −7.557 | 8.918 | 1.00 | 57.76 | N |
| ATOM | 1041 | NH2 | ARG | A | 64 | 61.220 | −6.268 | 8.594 | 1.00 | 59.82 | N |
| ATOM | 1044 | C | ARG | A | 64 | 55.451 | −2.492 | 9.608 | 1.00 | 51.03 | C |
| ATOM | 1045 | O | ARG | A | 64 | 56.259 | −2.219 | 8.721 | 1.00 | 53.48 | O |
| ATOM | 1046 | N | LYS | A | 65 | 54.198 | −2.035 | 9.584 | 1.00 | 53.47 | N |
| ATOM | 1048 | CA | LYS | A | 65 | 53.686 | −1.175 | 8.489 | 1.00 | 51.33 | C |
| ATOM | 1050 | CB | LYS | A | 65 | 53.329 | −2.050 | 7.229 | 1.00 | 48.60 | C |
| ATOM | 1053 | CG | LYS | A | 65 | 52.131 | −1.667 | 6.219 | 1.00 | 48.91 | C |
| ATOM | 1056 | CD | LYS | A | 65 | 51.911 | −2.702 | 4.881 | 1.00 | 50.16 | C |
| ATOM | 1059 | CE | LYS | A | 65 | 50.792 | −4.019 | 4.900 | 1.00 | 44.00 | C |
| ATOM | 1062 | NZ | LYS | A | 65 | 51.193 | −5.488 | 4.701 | 1.00 | 11.49 | N |
| ATOM | 1066 | C | LYS | A | 65 | 52.519 | −0.436 | 9.136 | 1.00 | 50.12 | C |
| ATOM | 1067 | O | LYS | A | 65 | 51.705 | −1.075 | 9.781 | 1.00 | 56.99 | O |
| ATOM | 1068 | N | GLY | A | 66 | 52.450 | 0.890 | 9.005 | 1.00 | 47.93 | N |
| ATOM | 1070 | CA | GLY | A | 66 | 51.359 | 1.678 | 9.588 | 1.00 | 45.56 | C |
| ATOM | 1073 | C | GLY | A | 66 | 50.196 | 1.971 | 8.628 | 1.00 | 42.47 | C |
| ATOM | 1074 | O | GLY | A | 66 | 50.277 | 1.787 | 7.418 | 1.00 | 40.89 | O |
| ATOM | 1075 | N | VAL | A | 67 | 49.095 | 2.470 | 9.152 | 1.00 | 39.98 | N |
| ATOM | 1077 | CA | VAL | A | 67 | 48.000 | 2.820 | 8.264 | 1.00 | 35.53 | C |
| ATOM | 1079 | CB | VAL | A | 67 | 47.307 | 1.531 | 7.793 | 1.00 | 35.43 | C |
| ATOM | 1081 | CG1 | VAL | A | 67 | 46.878 | 0.663 | 8.984 | 1.00 | 36.95 | C |
| ATOM | 1085 | CG2 | VAL | A | 67 | 46.129 | 1.806 | 6.999 | 1.00 | 33.52 | C |
| ATOM | 1089 | C | VAL | A | 67 | 47.021 | 3.841 | 8.882 | 1.00 | 40.42 | C |
| ATOM | 1090 | O | VAL | A | 67 | 46.885 | 3.969 | 10.118 | 1.00 | 40.64 | O |
| ATOM | 1091 | N | TYR | A | 68 | 46.352 | 4.546 | 7.971 | 1.00 | 45.72 | N |
| ATOM | 1093 | CA | TYR | A | 68 | 45.436 | 5.668 | 8.208 | 1.00 | 46.34 | C |
| ATOM | 1095 | CB | TYR | A | 68 | 46.052 | 6.874 | 7.536 | 1.00 | 51.19 | C |
| ATOM | 1098 | CG | TYR | A | 68 | 45.306 | 8.202 | 7.533 | 1.00 | 61.31 | C |
| ATOM | 1099 | CD1 | TYR | A | 68 | 44.911 | 8.826 | 8.726 | 1.00 | 62.21 | C |
| ATOM | 1101 | CE1 | TYR | A | 68 | 44.266 | 10.043 | 8.702 | 1.00 | 61.39 | C |
| ATOM | 1103 | CZ | TYR | A | 68 | 44.008 | 10.665 | 7.491 | 1.00 | 63.25 | C |
| ATOM | 1104 | OH | TYR | A | 68 | 43.361 | 11.886 | 7.472 | 1.00 | 66.32 | O |
| ATOM | 1106 | CE2 | TYR | A | 68 | 44.390 | 10.080 | 6.298 | 1.00 | 58.19 | C |
| ATOM | 1108 | CD2 | TYR | A | 68 | 45.039 | 8.864 | 6.321 | 1.00 | 61.27 | C |
| ATOM | 1110 | C | TYR | A | 68 | 44.152 | 5.440 | 7.505 | 1.00 | 41.55 | C |
| ATOM | 1111 | O | TYR | A | 68 | 44.130 | 5.185 | 6.335 | 1.00 | 42.23 | O |
| ATOM | 1112 | N | VAL | A | 69 | 43.045 | 5.557 | 8.195 | 1.00 | 45.73 | N |
| ATOM | 1114 | CA | VAL | A | 69 | 41.791 | 5.348 | 7.519 | 1.00 | 43.18 | C |
| ATOM | 1116 | CB | VAL | A | 69 | 41.256 | 3.958 | 7.920 | 1.00 | 46.99 | C |
| ATOM | 1118 | CG1 | VAL | A | 69 | 39.856 | 3.712 | 7.399 | 1.00 | 46.87 | C |
| ATOM | 1122 | CG2 | VAL | A | 69 | 42.216 | 2.897 | 7.399 | 1.00 | 39.84 | C |
| ATOM | 1126 | C | VAL | A | 69 | 40.831 | 6.478 | 7.832 | 1.00 | 37.88 | C |
| ATOM | 1127 | O | VAL | A | 69 | 40.436 | 6.622 | 8.962 | 1.00 | 45.27 | O |
| ATOM | 1128 | N | PRO | A | 70 | 40.497 | 7.286 | 6.823 | 1.00 | 34.02 | N |
| ATOM | 1129 | CA | PRO | A | 70 | 39.507 | 8.345 | 6.917 | 1.00 | 29.99 | C |
| ATOM | 1131 | CB | PRO | A | 70 | 40.071 | 9.345 | 5.934 | 1.00 | 26.73 | C |
| ATOM | 1134 | CG | PRO | A | 70 | 40.414 | 8.518 | 4.800 | 1.00 | 25.36 | C |
| ATOM | 1137 | CD | PRO | A | 70 | 41.047 | 7.267 | 5.456 | 1.00 | 34.53 | C |
| ATOM | 1140 | C | PRO | A | 70 | 38.118 | 7.941 | 6.383 | 1.00 | 35.42 | C |
| ATOM | 1141 | O | PRO | A | 70 | 37.992 | 7.309 | 5.318 | 1.00 | 35.66 | O |
| ATOM | 1142 | N | TYR | A | 71 | 37.067 | 8.315 | 7.102 | 1.00 | 43.55 | N |
| ATOM | 1144 | CA | TYR | A | 71 | 35.699 | 8.076 | 6.626 | 1.00 | 47.40 | C |
| ATOM | 1146 | CB | TYR | A | 71 | 34.943 | 7.305 | 7.684 | 1.00 | 48.07 | C |
| ATOM | 1149 | CG | TYR | A | 71 | 35.651 | 6.025 | 8.159 | 1.00 | 49.56 | C |
| ATOM | 1150 | CD1 | TYR | A | 71 | 35.181 | 4.750 | 7.818 | 1.00 | 49.21 | C |
| ATOM | 1152 | CE1 | TYR | A | 71 | 35.822 | 3.609 | 8.258 | 1.00 | 46.98 | C |
| ATOM | 1154 | CZ | TYR | A | 71 | 36.924 | 3.741 | 9.045 | 1.00 | 44.80 | C |
| ATOM | 1155 | OH | TYR | A | 71 | 37.601 | 2.666 | 9.526 | 1.00 | 46.04 | O |
| ATOM | 1157 | CE2 | TYR | A | 71 | 37.387 | 4.972 | 9.389 | 1.00 | 50.64 | C |
| ATOM | 1159 | CD2 | TYR | A | 71 | 36.756 | 6.097 | 8.949 | 1.00 | 50.58 | C |
| ATOM | 1161 | C | TYR | A | 71 | 34.974 | 9.409 | 6.253 | 1.00 | 51.01 | C |
| ATOM | 1162 | O | TYR | A | 71 | 35.607 | 10.448 | 6.041 | 1.00 | 56.69 | O |
| ATOM | 1163 | N | THR | A | 72 | 33.653 | 9.373 | 6.144 | 1.00 | 54.06 | N |
| ATOM | 1165 | CA | THR | A | 72 | 32.842 | 10.586 | 5.982 | 1.00 | 48.48 | C |
| ATOM | 1167 | CB | THR | A | 72 | 31.462 | 10.140 | 5.541 | 1.00 | 42.95 | C |
| ATOM | 1169 | OG1 | THR | A | 72 | 31.614 | 9.334 | 4.387 | 1.00 | 44.35 | O |
| ATOM | 1171 | CG2 | THR | A | 72 | 30.620 | 11.232 | 4.935 | 1.00 | 46.38 | C |
| ATOM | 1175 | C | THR | A | 72 | 32.834 | 11.426 | 7.291 | 1.00 | 50.73 | C |
| ATOM | 1176 | O | THR | A | 72 | 32.793 | 12.633 | 7.256 | 1.00 | 49.87 | O |
| ATOM | 1177 | N | GLN | A | 73 | 32.873 | 10.790 | 8.446 | 1.00 | 57.02 | N |
| ATOM | 1179 | CA | GLN | A | 73 | 33.020 | 11.507 | 9.717 | 1.00 | 61.36 | C |
| ATOM | 1181 | CB | GLN | A | 73 | 31.704 | 11.705 | 10.489 | 1.00 | 61.87 | C |
| ATOM | 1184 | CG | GLN | A | 73 | 30.932 | 13.008 | 10.237 | 1.00 | 69.07 | C |
| ATOM | 1187 | CD | GLN | A | 73 | 31.616 | 14.250 | 10.830 | 1.00 | 73.52 | C |
| ATOM | 1188 | OE1 | GLN | A | 73 | 31.469 | 14.561 | 12.028 | 1.00 | 73.43 | O |
| ATOM | 1189 | NE2 | GLN | A | 73 | 32.369 | 14.960 | 9.987 | 1.00 | 74.63 | N |
| ATOM | 1192 | C | GLN | A | 73 | 33.930 | 10.654 | 10.576 | 1.00 | 61.53 | C |
| ATOM | 1193 | O | GLN | A | 73 | 33.592 | 9.520 | 10.910 | 1.00 | 63.25 | O |
| ATOM | 1194 | N | GLY | A | 74 | 35.079 | 11.208 | 10.935 | 1.00 | 62.04 | N |
| ATOM | 1196 | CA | GLY | A | 74 | 36.019 | 10.538 | 11.816 | 1.00 | 60.66 | C |
| ATOM | 1199 | C | GLY | A | 74 | 37.207 | 9.927 | 11.093 | 1.00 | 54.01 | C |
| ATOM | 1200 | O | GLY | A | 74 | 37.126 | 9.641 | 9.901 | 1.00 | 47.51 | O |
| ATOM | 1201 | N | LYS | A | 75 | 38.310 | 9.754 | 11.822 | 1.00 | 51.55 | N |
| ATOM | 1203 | CA | LYS | A | 75 | 39.445 | 8.982 | 11.330 | 1.00 | 48.56 | C |
| ATOM | 1205 | CB | LYS | A | 75 | 40.395 | 9.783 | 10.472 | 1.00 | 49.33 | C |
| ATOM | 1208 | CG | LYS | A | 75 | 40.953 | 11.042 | 11.076 | 1.00 | 50.33 | C |
| ATOM | 1211 | CD | LYS | A | 75 | 41.467 | 11.932 | 9.917 | 1.00 | 49.25 | C |
| ATOM | 1214 | CE | LYS | A | 75 | 41.609 | 13.425 | 10.286 | 1.00 | 50.50 | C |
| ATOM | 1217 | NZ | LYS | A | 75 | 43.031 | 13.815 | 10.654 | 1.00 | 42.60 | N |
| ATOM | 1221 | C | LYS | A | 75 | 40.203 | 8.425 | 12.455 | 1.00 | 45.88 | C |
| ATOM | 1222 | O | LYS | A | 75 | 39.958 | 8.795 | 13.599 | 1.00 | 46.47 | O |
| ATOM | 1223 | N | TRP | A | 76 | 41.105 | 7.501 | 12.110 | 1.00 | 46.31 | N |
| ATOM | 1225 | CA | TRP | A | 76 | 42.052 | 6.881 | 13.058 | 1.00 | 47.24 | C |
| ATOM | 1227 | CB | TRP | A | 76 | 41.502 | 5.620 | 13.777 | 1.00 | 41.96 | C |
| ATOM | 1230 | CG | TRP | A | 76 | 40.864 | 4.653 | 12.875 | 1.00 | 40.56 | C |
| ATOM | 1231 | CD1 | TRP | A | 76 | 39.552 | 4.578 | 12.592 | 1.00 | 42.47 | C |
| ATOM | 1233 | NE1 | TRP | A | 76 | 39.304 | 3.557 | 11.706 | 1.00 | 44.26 | N |
| ATOM | 1235 | CE2 | TRP | A | 76 | 40.488 | 2.949 | 11.407 | 1.00 | 40.38 | C |
| ATOM | 1236 | CD2 | TRP | A | 76 | 41.494 | 3.615 | 12.132 | 1.00 | 39.72 | C |
| ATOM | 1237 | CE3 | TRP | A | 76 | 42.814 | 3.176 | 11.996 | 1.00 | 43.23 | C |
| ATOM | 1239 | CZ3 | TRP | A | 76 | 43.082 | 2.116 | 11.157 | 1.00 | 34.09 | C |
| ATOM | 1241 | CH2 | TRP | A | 76 | 42.047 | 1.481 | 10.452 | 1.00 | 40.84 | C |
| ATOM | 1243 | CZ2 | TRP | A | 76 | 40.749 | 1.887 | 10.567 | 1.00 | 37.49 | C |
| ATOM | 1245 | C | TRP | A | 76 | 43.352 | 6.572 | 12.283 | 1.00 | 46.65 | C |
| ATOM | 1246 | O | TRP | A | 76 | 43.376 | 6.593 | 11.058 | 1.00 | 44.54 | O |
| ATOM | 1247 | N | GLU | A | 77 | 44.417 | 6.302 | 13.025 | 1.00 | 44.45 | N |
| ATOM | 1249 | CA | GLU | A | 77 | 45.726 | 6.056 | 12.465 | 1.00 | 49.78 | C |
| ATOM | 1251 | CB | GLU | A | 77 | 46.637 | 7.292 | 12.696 | 1.00 | 54.17 | C |
| ATOM | 1254 | CG | GLU | A | 77 | 45.798 | 8.559 | 12.966 | 1.00 | 63.47 | C |
| ATOM | 1257 | CD | GLU | A | 77 | 46.260 | 9.861 | 12.296 | 1.00 | 65.72 | C |
| ATOM | 1258 | OE1 | GLU | A | 77 | 47.499 | 10.099 | 12.217 | 1.00 | 62.94 | O |
| ATOM | 1259 | OE2 | GLU | A | 77 | 45.341 | 10.642 | 11.855 | 1.00 | 57.77 | O |
| ATOM | 1260 | C | GLU | A | 77 | 46.145 | 4.851 | 13.268 | 1.00 | 48.59 | C |
| ATOM | 1261 | O | GLU | A | 77 | 45.835 | 4.787 | 14.477 | 1.00 | 46.46 | O |
| ATOM | 1262 | N | GLY | A | 78 | 46.814 | 3.881 | 12.643 | 1.00 | 44.59 | N |
| ATOM | 1264 | CA | GLY | A | 78 | 47.148 | 2.670 | 13.387 | 1.00 | 43.33 | C |
| ATOM | 1267 | C | GLY | A | 78 | 48.378 | 1.905 | 12.935 | 1.00 | 45.34 | C |
| ATOM | 1268 | O | GLY | A | 78 | 49.054 | 2.322 | 11.945 | 1.00 | 46.85 | O |
| ATOM | 1269 | N | GLU | A | 79 | 48.635 | 0.805 | 13.657 | 1.00 | 40.38 | N |
| ATOM | 1271 | CA | GLU | A | 79 | 49.702 | −0.144 | 13.391 | 1.00 | 45.02 | C |
| ATOM | 1273 | CB | GLU | A | 79 | 50.631 | −0.248 | 14.675 | 1.00 | 51.34 | C |
| ATOM | 1276 | CG | GLU | A | 79 | 51.478 | 1.000 | 15.101 | 1.00 | 55.46 | C |
| ATOM | 1279 | CD | GLU | A | 79 | 52.047 | 0.912 | 16.565 | 1.00 | 65.73 | C |
| ATOM | 1280 | OE1 | GLU | A | 79 | 52.921 | 1.815 | 17.021 | 1.00 | 64.66 | O |
| ATOM | 1281 | OE2 | GLU | A | 79 | 51.612 | −0.082 | 17.271 | 1.00 | 54.33 | O |
| ATOM | 1282 | C | GLU | A | 79 | 49.162 | −1.565 | 12.952 | 1.00 | 44.43 | C |
| ATOM | 1283 | O | GLU | A | 79 | 48.318 | −2.183 | 13.626 | 1.00 | 39.67 | O |
| ATOM | 1284 | N | LEU | A | 80 | 49.666 | −2.080 | 11.829 | 1.00 | 45.33 | N |
| ATOM | 1286 | CA | LEU | A | 80 | 49.240 | −3.386 | 11.250 | 1.00 | 42.70 | C |
| ATOM | 1288 | CB | LEU | A | 80 | 49.454 | −3.355 | 9.743 | 1.00 | 39.42 | C |
| ATOM | 1291 | CG | LEU | A | 80 | 48.461 | −2.502 | 9.024 | 1.00 | 38.72 | C |
| ATOM | 1293 | CD1 | LEU | A | 80 | 48.671 | −2.614 | 7.556 | 1.00 | 41.48 | C |
| ATOM | 1297 | CD2 | LEU | A | 80 | 47.097 | −2.967 | 9.427 | 1.00 | 43.39 | C |
| ATOM | 1301 | C | LEU | A | 80 | 49.908 | −4.689 | 11.737 | 1.00 | 42.16 | C |
| ATOM | 1302 | O | LEU | A | 80 | 51.133 | −4.789 | 11.947 | 1.00 | 39.15 | O |
| ATOM | 1303 | N | GLY | A | 81 | 49.084 | −5.715 | 11.852 | 1.00 | 38.15 | N |
| ATOM | 1305 | CA | GLY | A | 81 | 49.519 | −6.972 | 12.414 | 1.00 | 40.41 | C |
| ATOM | 1308 | C | GLY | A | 81 | 48.517 | −8.073 | 12.156 | 1.00 | 41.89 | C |
| ATOM | 1309 | O | GLY | A | 81 | 47.680 | −7.948 | 11.282 | 1.00 | 40.94 | O |
| ATOM | 1310 | N | THR | A | 82 | 48.631 | −9.167 | 12.898 | 1.00 | 45.69 | N |
| ATOM | 1312 | CA | THR | A | 82 | 47.663 | −10.265 | 12.824 | 1.00 | 41.72 | C |
| ATOM | 1314 | CB | THR | A | 82 | 48.071 | −11.342 | 11.837 | 1.00 | 41.41 | C |
| ATOM | 1316 | OG1 | THR | A | 82 | 49.268 | −12.009 | 12.302 | 1.00 | 40.17 | O |
| ATOM | 1318 | CG2 | THR | A | 82 | 48.423 | −10.774 | 10.506 | 1.00 | 39.10 | C |
| ATOM | 1322 | C | THR | A | 82 | 47.570 | −10.947 | 14.176 | 1.00 | 42.06 | C |
| ATOM | 1323 | O | THR | A | 82 | 48.416 | −10.779 | 15.077 | 1.00 | 41.59 | O |
| ATOM | 1324 | N | ASP | A | 83 | 46.532 | −11.736 | 14.315 | 1.00 | 39.43 | N |
| ATOM | 1326 | CA | ASP | A | 83 | 46.344 | −12.449 | 15.538 | 1.00 | 41.41 | C |
| ATOM | 1328 | CB | ASP | A | 83 | 46.090 | −11.482 | 16.675 | 1.00 | 40.00 | C |
| ATOM | 1331 | CG | ASP | A | 83 | 46.263 | −12.135 | 18.011 | 1.00 | 44.18 | C |
| ATOM | 1332 | OD1 | ASP | A | 83 | 46.475 | −13.344 | 18.014 | 1.00 | 45.61 | O |
| ATOM | 1333 | OD2 | ASP | A | 83 | 46.225 | −11.551 | 19.111 | 1.00 | 52.07 | O |
| ATOM | 1334 | C | ASP | A | 83 | 45.163 | −13.314 | 15.246 | 1.00 | 44.90 | C |
| ATOM | 1335 | O | ASP | A | 83 | 44.544 | −13.136 | 14.216 | 1.00 | 51.63 | O |
| ATOM | 1336 | N | LEU | A | 84 | 44.847 | −14.260 | 16.110 | 1.00 | 45.58 | N |
| ATOM | 1338 | CA | LEU | A | 84 | 43.699 | −15.139 | 15.877 | 1.00 | 44.62 | C |
| ATOM | 1340 | CB | LEU | A | 84 | 43.951 | −16.446 | 16.629 | 1.00 | 41.76 | C |
| ATOM | 1343 | CG | LEU | A | 84 | 45.078 | −17.361 | 16.133 | 1.00 | 41.81 | C |
| ATOM | 1345 | CD1 | LEU | A | 84 | 45.177 | −18.491 | 17.103 | 1.00 | 46.46 | C |
| ATOM | 1349 | CD2 | LEU | A | 84 | 44.847 | −17.978 | 14.767 | 1.00 | 42.92 | C |
| ATOM | 1353 | C | LEU | A | 84 | 42.374 | −14.468 | 16.315 | 1.00 | 41.20 | C |
| ATOM | 1354 | O | LEU | A | 84 | 42.388 | −13.643 | 17.191 | 1.00 | 52.36 | O |
| ATOM | 1355 | N | VAL | A | 85 | 41.234 | −14.803 | 15.719 | 1.00 | 41.48 | N |
| ATOM | 1357 | CA | VAL | A | 85 | 39.973 | −14.143 | 16.080 | 1.00 | 37.87 | C |
| ATOM | 1359 | CB | VAL | A | 85 | 39.743 | −12.994 | 15.120 | 1.00 | 41.53 | C |
| ATOM | 1361 | CG1 | VAL | A | 85 | 38.437 | −12.345 | 15.380 | 1.00 | 47.66 | C |
| ATOM | 1365 | CG2 | VAL | A | 85 | 40.822 | −11.985 | 15.229 | 1.00 | 41.79 | C |
| ATOM | 1369 | C | VAL | A | 85 | 38.680 | −14.998 | 16.052 | 1.00 | 39.48 | C |
| ATOM | 1370 | O | VAL | A | 85 | 38.448 | −15.737 | 15.125 | 1.00 | 44.43 | O |
| ATOM | 1371 | N | SER | A | 86 | 37.833 | −14.882 | 17.068 | 1.00 | 40.31 | N |
| ATOM | 1373 | CA | SER | A | 86 | 36.564 | −15.608 | 17.085 | 1.00 | 44.63 | C |
| ATOM | 1375 | CB | SER | A | 86 | 36.474 | −16.510 | 18.316 | 1.00 | 41.82 | C |
| ATOM | 1378 | OG | SER | A | 86 | 37.738 | −17.097 | 18.545 | 1.00 | 55.30 | O |
| ATOM | 1380 | C | SER | A | 86 | 35.365 | −14.678 | 17.157 | 1.00 | 45.48 | C |
| ATOM | 1381 | O | SER | A | 86 | 35.525 | −13.486 | 17.463 | 1.00 | 41.16 | O |
| ATOM | 1382 | N | ILE | A | 87 | 34.185 | −15.266 | 16.888 | 1.00 | 43.04 | N |
| ATOM | 1384 | CA | ILE | A | 87 | 32.885 | −14.643 | 17.083 | 1.00 | 41.10 | C |
| ATOM | 1386 | CB | ILE | A | 87 | 32.215 | −14.421 | 15.706 | 1.00 | 42.65 | C |
| ATOM | 1388 | CG1 | ILE | A | 87 | 33.234 | −13.839 | 14.754 | 1.00 | 43.78 | C |
| ATOM | 1391 | CD1 | ILE | A | 87 | 32.624 | −13.331 | 13.496 | 1.00 | 47.44 | C |
| ATOM | 1395 | CG2 | ILE | A | 87 | 31.050 | −13.435 | 15.795 | 1.00 | 39.53 | C |
| ATOM | 1399 | C | ILE | A | 87 | 32.022 | −15.517 | 18.062 | 1.00 | 43.32 | C |
| ATOM | 1400 | O | ILE | A | 87 | 31.287 | −16.462 | 17.663 | 1.00 | 44.61 | O |
| ATOM | 1401 | N | PRO | A | 88 | 32.120 | −15.212 | 19.346 | 1.00 | 41.82 | N |
| ATOM | 1402 | CA | PRO | A | 88 | 31.440 | −16.004 | 20.380 | 1.00 | 45.42 | C |
| ATOM | 1404 | CB | PRO | A | 88 | 31.311 | −15.040 | 21.563 | 1.00 | 46.34 | C |
| ATOM | 1407 | CG | PRO | A | 88 | 32.594 | −14.147 | 21.446 | 1.00 | 46.41 | C |
| ATOM | 1410 | CD | PRO | A | 88 | 32.902 | −14.107 | 19.925 | 1.00 | 46.82 | C |
| ATOM | 1413 | C | PRO | A | 88 | 30.103 | −16.508 | 19.949 | 1.00 | 44.85 | C |
| ATOM | 1414 | O | PRO | A | 88 | 29.867 | −17.685 | 20.208 | 1.00 | 53.97 | O |
| ATOM | 1415 | N | HIS | A | 89 | 29.243 | −15.692 | 19.354 | 1.00 | 46.76 | N |
| ATOM | 1417 | CA | HIS | A | 89 | 27.981 | −16.214 | 18.801 | 1.00 | 50.05 | C |
| ATOM | 1419 | CB | HIS | A | 89 | 26.757 | −15.526 | 19.316 | 1.00 | 48.45 | C |
| ATOM | 1422 | CG | HIS | A | 89 | 26.753 | −15.317 | 20.780 | 1.00 | 55.56 | C |
| ATOM | 1423 | ND1 | HIS | A | 89 | 26.375 | −16.302 | 21.664 | 1.00 | 60.41 | N |
| ATOM | 1425 | CE1 | HIS | A | 89 | 26.460 | −15.830 | 22.899 | 1.00 | 64.25 | C |
| ATOM | 1427 | NE2 | HIS | A | 89 | 26.878 | −14.576 | 22.842 | 1.00 | 59.22 | N |
| ATOM | 1429 | CD2 | HIS | A | 89 | 27.065 | −14.229 | 21.524 | 1.00 | 56.69 | C |
| ATOM | 1431 | C | HIS | A | 89 | 27.958 | −15.966 | 17.339 | 1.00 | 54.03 | C |
| ATOM | 1432 | O | HIS | A | 89 | 27.124 | −15.179 | 16.853 | 1.00 | 55.54 | O |
| ATOM | 1433 | N | GLY | A | 90 | 28.896 | −16.606 | 16.653 | 1.00 | 55.69 | N |
| ATOM | 1435 | CA | GLY | A | 90 | 28.986 | −16.585 | 15.201 | 1.00 | 55.66 | C |
| ATOM | 1438 | C | GLY | A | 90 | 29.407 | −18.001 | 14.865 | 1.00 | 52.28 | C |
| ATOM | 1439 | O | GLY | A | 90 | 29.482 | −18.847 | 15.746 | 1.00 | 62.08 | O |
| ATOM | 1440 | N | PRO | A | 91 | 29.704 | −18.297 | 13.628 | 1.00 | 47.23 | N |
| ATOM | 1441 | CA | PRO | A | 91 | 30.069 | −19.664 | 13.308 | 1.00 | 47.93 | C |
| ATOM | 1443 | CB | PRO | A | 91 | 30.399 | −19.586 | 11.830 | 1.00 | 49.04 | C |
| ATOM | 1446 | CG | PRO | A | 91 | 30.611 | −18.114 | 11.561 | 1.00 | 45.67 | C |
| ATOM | 1449 | CD | PRO | A | 91 | 29.727 | −17.419 | 12.456 | 1.00 | 48.30 | C |
| ATOM | 1452 | C | PRO | A | 91 | 31.248 | −19.980 | 14.206 | 1.00 | 54.34 | C |
| ATOM | 1453 | O | PRO | A | 91 | 32.052 | −19.090 | 14.448 | 1.00 | 56.82 | O |
| ATOM | 1454 | N | GLN | A | 92 | 31.343 | −21.211 | 14.691 | 1.00 | 54.83 | N |
| ATOM | 1456 | CA | GLN | A | 92 | 32.261 | −21.569 | 15.765 | 1.00 | 55.86 | C |
| ATOM | 1458 | CB | GLN | A | 92 | 31.662 | −22.754 | 16.503 | 1.00 | 60.18 | C |
| ATOM | 1461 | CG | GLN | A | 92 | 30.322 | −22.429 | 17.101 | 1.00 | 58.46 | C |
| ATOM | 1464 | CD | GLN | A | 92 | 30.470 | −21.890 | 18.493 | 1.00 | 64.81 | C |
| ATOM | 1465 | OE1 | GLN | A | 92 | 30.847 | −22.631 | 19.418 | 1.00 | 63.91 | O |
| ATOM | 1466 | NE2 | GLN | A | 92 | 30.178 | −20.591 | 18.662 | 1.00 | 67.59 | N |
| ATOM | 1469 | C | GLN | A | 92 | 33.635 | −21.950 | 15.325 | 1.00 | 56.89 | C |
| ATOM | 1470 | O | GLN | A | 92 | 34.036 | −23.111 | 15.411 | 1.00 | 62.48 | O |
| ATOM | 1471 | N | VAL | A | 93 | 34.387 | −20.961 | 14.897 | 1.00 | 55.14 | N |
| ATOM | 1473 | CA | VAL | A | 93 | 35.629 | −21.223 | 14.237 | 1.00 | 50.07 | C |
| ATOM | 1475 | CB | VAL | A | 93 | 35.426 | −21.112 | 12.760 | 1.00 | 51.39 | C |
| ATOM | 1477 | CG1 | VAL | A | 93 | 34.450 | −22.169 | 12.285 | 1.00 | 53.66 | C |
| ATOM | 1481 | CG2 | VAL | A | 93 | 34.902 | −19.715 | 12.424 | 1.00 | 53.54 | C |
| ATOM | 1485 | C | VAL | A | 93 | 36.605 | −20.174 | 14.620 | 1.00 | 54.79 | C |
| ATOM | 1486 | O | VAL | A | 93 | 36.303 | −19.243 | 15.382 | 1.00 | 56.10 | O |
| ATOM | 1487 | N | THR | A | 94 | 37.805 | −20.303 | 14.089 | 1.00 | 53.29 | N |
| ATOM | 1489 | CA | THR | A | 94 | 38.802 | −19.378 | 14.467 | 1.00 | 52.14 | C |
| ATOM | 1491 | CB | THR | A | 94 | 39.464 | −19.960 | 15.690 | 1.00 | 53.16 | C |
| ATOM | 1493 | OG1 | TUR | A | 94 | 38.506 | −19.888 | 16.761 | 1.00 | 53.79 | O |
| ATOM | 1495 | CG2 | THR | A | 94 | 40.645 | −19.093 | 16.197 | 1.00 | 56.24 | C |
| ATOM | 1499 | C | THR | A | 94 | 39.688 | −19.131 | 13.285 | 1.00 | 52.21 | C |
| ATOM | 1500 | O | THR | A | 94 | 40.148 | −20.051 | 12.624 | 1.00 | 61.76 | O |
| ATOM | 1501 | N | VAL | A | 95 | 39.923 | −17.867 | 12.993 | 1.00 | 51.10 | N |
| ATOM | 1503 | CA | VAL | A | 95 | 40.645 | −17.543 | 11.772 | 1.00 | 48.01 | C |
| ATOM | 1505 | CB | VAL | A | 95 | 39.722 | −16.862 | 10.706 | 1.00 | 43.19 | C |
| ATOM | 1507 | CG1 | VAL | A | 95 | 38.322 | −17.270 | 10.858 | 1.00 | 37.56 | C |
| ATOM | 1511 | CG2 | VAL | A | 95 | 39.760 | −15.377 | 10.824 | 1.00 | 47.32 | C |
| ATOM | 1515 | C | VAL | A | 95 | 41.736 | −16.577 | 12.111 | 1.00 | 45.91 | C |
| ATOM | 1516 | O | VAL | A | 95 | 41.608 | −15.811 | 13.065 | 1.00 | 50.89 | O |
| ATOM | 1517 | N | ARG | A | 96 | 42.814 | −16.615 | 11.353 | 1.00 | 41.67 | N |
| ATOM | 1519 | CA | ARG | A | 96 | 43.788 | −15.565 | 11.502 | 1.00 | 43.07 | C |
| ATOM | 1521 | CB | ARG | A | 96 | 45.180 | −16.043 | 11.235 | 1.00 | 41.47 | C |
| ATOM | 1524 | CG | ARG | A | 96 | 46.175 | −14.982 | 11.546 | 1.00 | 41.13 | C |
| ATOM | 1527 | CD | ARG | A | 96 | 47.579 | −15.400 | 11.409 | 1.00 | 43.93 | C |
| ATOM | 1530 | NE | ARG | A | 96 | 48.156 | −15.492 | 12.744 | 1.00 | 52.62 | N |
| ATOM | 1532 | CZ | ARG | A | 96 | 48.450 | −16.621 | 13.334 | 1.00 | 56.20 | C |
| ATOM | 1533 | NH1 | ARG | A | 96 | 48.233 | −17.778 | 12.696 | 1.00 | 59.54 | N |
| ATOM | 1536 | NH2 | ARG | A | 96 | 48.965 | −16.594 | 14.565 | 1.00 | 58.71 | N |
| ATOM | 1539 | C | ARG | A | 96 | 43.441 | −14.460 | 10.512 | 1.00 | 40.12 | C |
| ATOM | 1540 | O | ARG | A | 96 | 43.081 | −14.756 | 9.368 | 1.00 | 46.93 | O |
| ATOM | 1541 | N | ALA | A | 97 | 43.539 | −13.208 | 10.963 | 1.00 | 37.94 | N |
| ATOM | 1543 | CA | ALA | A | 97 | 43.294 | −12.033 | 10.119 | 1.00 | 40.48 | C |
| ATOM | 1545 | CB | ALA | A | 97 | 41.818 | −11.575 | 10.255 | 1.00 | 40.89 | C |
| ATOM | 1549 | C | ALA | A | 97 | 44.193 | −10.823 | 10.362 | 1.00 | 38.63 | C |
| ATOM | 1550 | O | ALA | A | 97 | 44.823 | −10.673 | 11.393 | 1.00 | 40.23 | O |
| ATOM | 1551 | N | ASN | A | 98 | 44.208 | −9.957 | 9.358 | 1.00 | 41.67 | N |
| ATOM | 1553 | CA | ASN | A | 98 | 44.823 | −8.658 | 9.427 | 1.00 | 39.41 | C |
| ATOM | 1555 | CB | ASN | A | 98 | 44.680 | −7.992 | 8.094 | 1.00 | 42.95 | C |
| ATOM | 1558 | CG | ASN | A | 98 | 45.476 | −8.649 | 7.070 | 1.00 | 37.46 | C |
| ATOM | 1559 | OD1 | ASN | A | 98 | 46.593 | −8.980 | 7.320 | 1.00 | 37.53 | O |
| ATOM | 1560 | ND2 | ASN | A | 98 | 44.915 | −8.841 | 5.896 | 1.00 | 52.61 | N |
| ATOM | 1563 | C | ASN | A | 98 | 44.083 | −7.799 | 10.369 | 1.00 | 40.10 | C |
| ATOM | 1564 | O | ASN | A | 98 | 42.863 | −7.864 | 10.434 | 1.00 | 41.02 | O |
| ATOM | 1565 | N | ILE | A | 99 | 44.808 | −6.962 | 11.089 | 1.00 | 44.12 | N |
| ATOM | 1567 | CA | ILE | A | 99 | 44.198 | −6.153 | 12.128 | 1.00 | 44.32 | C |
| ATOM | 1569 | CB | ILE | A | 99 | 44.298 | −6.840 | 13.502 | 1.00 | 41.75 | C |
| ATOM | 1571 | CG1 | ILE | A | 99 | 43.507 | −8.127 | 13.511 | 1.00 | 43.97 | C |
| ATOM | 1574 | CD1 | ILE | A | 99 | 43.588 | −8.918 | 14.872 | 1.00 | 48.22 | C |
| ATOM | 1578 | CG2 | ILE | A | 99 | 43.722 | −5.928 | 14.602 | 1.00 | 45.32 | C |
| ATOM | 1582 | C | ILE | A | 99 | 44.932 | −4.844 | 12.154 | 1.00 | 44.51 | C |
| ATOM | 1583 | O | ILE | A | 99 | 46.082 | −4.777 | 11.783 | 1.00 | 47.40 | O |
| ATOM | 1584 | N | ALA | A | 100 | 44.252 | −3.795 | 12.579 | 1.00 | 45.36 | N |
| ATOM | 1586 | CA | ALA | A | 100 | 44.880 | −2.510 | 12.655 | 1.00 | 41.65 | C |
| ATOM | 1588 | CB | ALA | A | 100 | 44.230 | −1.553 | 11.762 | 1.00 | 41.33 | C |
| ATOM | 1592 | C | ALA | A | 100 | 44.732 | −2.068 | 14.054 | 1.00 | 42.41 | C |
| ATOM | 1593 | O | ALA | A | 100 | 43.612 | −1.861 | 14.531 | 1.00 | 41.57 | O |
| ATOM | 1594 | N | ALA | A | 101 | 45.865 | −1.951 | 14.736 | 1.00 | 40.77 | N |
| ATOM | 1596 | CA | ALA | A | 101 | 45.801 | −1.461 | 16.066 | 1.00 | 37.99 | C |
| ATOM | 1598 | CB | ALA | A | 101 | 46.907 | −1.896 | 16.841 | 1.00 | 44.45 | C |
| ATOM | 1602 | C | ALA | A | 101 | 45.819 | 0.007 | 15.881 | 1.00 | 41.73 | C |
| ATOM | 1603 | O | ALA | A | 101 | 46.801 | 0.611 | 15.432 | 1.00 | 40.15 | O |
| ATOM | 1604 | N | ILE | A | 102 | 44.656 | 0.544 | 16.204 | 1.00 | 42.71 | N |
| ATOM | 1606 | CA | ILE | A | 102 | 44.379 | 1.911 | 16.241 | 1.00 | 39.72 | C |
| ATOM | 1608 | CB | ILE | A | 102 | 43.010 | 2.009 | 16.549 | 1.00 | 44.46 | C |
| ATOM | 1610 | CG1 | ILE | A | 102 | 42.182 | 1.767 | 15.321 | 1.00 | 43.48 | C |
| ATOM | 1613 | CD1 | ILE | A | 102 | 40.768 | 1.961 | 15.644 | 1.00 | 40.40 | C |
| ATOM | 1617 | CG2 | ILE | A | 102 | 42.732 | 3.406 | 17.111 | 1.00 | 48.29 | C |
| ATOM | 1621 | C | ILE | A | 102 | 45.024 | 2.449 | 17.452 | 1.00 | 44.91 | C |
| ATOM | 1622 | O | ILE | A | 102 | 44.777 | 1.856 | 18.522 | 1.00 | 42.30 | O |
| ATOM | 1623 | N | THR | A | 103 | 45.795 | 3.544 | 17.257 | 1.00 | 45.74 | N |
| ATOM | 1625 | CA | THR | A | 103 | 46.583 | 4.269 | 18.265 | 1.00 | 48.02 | C |
| ATOM | 1627 | CB | THR | A | 103 | 48.113 | 4.118 | 17.971 | 1.00 | 48.61 | C |
| ATOM | 1629 | OG1 | THR | A | 103 | 48.365 | 4.329 | 16.578 | 1.00 | 53.38 | O |
| ATOM | 1631 | CG2 | THR | A | 103 | 48.601 | 2.733 | 18.209 | 1.00 | 42.44 | C |
| ATOM | 1635 | C | THR | A | 103 | 46.295 | 5.790 | 18.383 | 1.00 | 48.94 | C |
| ATOM | 1636 | O | THR | A | 103 | 46.547 | 6.358 | 19.407 | 1.00 | 51.09 | O |
| ATOM | 1637 | N | GLU | A | 104 | 45.804 | 6.456 | 17.343 | 1.00 | 54.39 | N |
| ATOM | 1639 | CA | GLU | A | 104 | 45.357 | 7.855 | 17.474 | 1.00 | 56.21 | C |
| ATOM | 1641 | CB | GLU | A | 104 | 46.402 | 8.823 | 16.940 | 1.00 | 61.91 | C |
| ATOM | 1644 | CG | GLU | A | 104 | 47.736 | 8.206 | 16.593 | 1.00 | 66.58 | C |
| ATOM | 1647 | CD | GLU | A | 104 | 48.599 | 9.142 | 15.758 | 1.00 | 71.04 | C |
| ATOM | 1648 | OE1 | GLU | A | 104 | 48.030 | 9.782 | 14.832 | 1.00 | 69.31 | O |
| ATOM | 1649 | OE2 | GLU | A | 104 | 49.833 | 9.228 | 16.037 | 1.00 | 69.74 | O |
| ATOM | 1650 | C | GLU | A | 104 | 44.062 | 8.088 | 16.684 | 1.00 | 53.27 | C |
| ATOM | 1651 | O | GLU | A | 104 | 43.890 | 7.496 | 15.610 | 1.00 | 44.69 | O |
| ATOM | 1652 | N | SER | A | 105 | 43.182 | 8.968 | 17.179 | 1.00 | 52.49 | N |
| ATOM | 1654 | CA | SER | A | 105 | 41.858 | 9.143 | 16.562 | 1.00 | 54.80 | C |
| ATOM | 1656 | CB | SER | A | 105 | 40.954 | 8.049 | 17.093 | 1.00 | 53.62 | C |
| ATOM | 1659 | OG | SER | A | 105 | 41.288 | 7.850 | 18.448 | 1.00 | 51.12 | O |
| ATOM | 1661 | C | SER | A | 105 | 41.136 | 10.454 | 16.809 | 1.00 | 56.75 | C |
| ATOM | 1662 | O | SER | A | 105 | 41.285 | 11.077 | 17.858 | 1.00 | 57.71 | O |
| ATOM | 1663 | N | ASP | A | 106 | 40.301 | 10.829 | 15.841 | 1.00 | 57.97 | N |
| ATOM | 1665 | CA | ASP | A | 106 | 39.596 | 12.109 | 15.846 | 1.00 | 57.67 | C |
| ATOM | 1667 | CB | ASP | A | 106 | 40.343 | 13.113 | 14.941 | 1.00 | 60.33 | C |
| ATOM | 1670 | CG | ASP | A | 106 | 39.862 | 14.562 | 15.098 | 1.00 | 66.40 | C |
| ATOM | 1671 | OD1 | ASP | A | 106 | 39.308 | 14.924 | 16.157 | 1.00 | 70.77 | O |
| ATOM | 1672 | OD2 | ASP | A | 106 | 40.007 | 15.422 | 14.192 | 1.00 | 70.32 | O |
| ATOM | 1673 | C | ASP | A | 106 | 38.136 | 11.950 | 15.391 | 1.00 | 54.92 | C |
| ATOM | 1674 | O | ASP | A | 106 | 37.824 | 11.896 | 14.204 | 1.00 | 46.42 | O |
| ATOM | 1675 | N | LYS | A | 107 | 37.248 | 11.869 | 16.371 | 1.00 | 57.47 | N |
| ATOM | 1677 | CA | LYS | A | 107 | 35.810 | 11.879 | 16.136 | 1.00 | 55.68 | C |
| ATOM | 1679 | CB | LYS | A | 107 | 35.365 | 13.092 | 15.277 | 1.00 | 55.19 | C |
| ATOM | 1682 | CG | LYS | A | 107 | 35.774 | 14.500 | 15.817 | 1.00 | 54.89 | C |
| ATOM | 1685 | CD | LYS | A | 107 | 35.904 | 15.570 | 14.683 | 1.00 | 57.62 | C |
| ATOM | 1688 | CE | LYS | A | 107 | 36.209 | 16.997 | 15.235 | 1.00 | 62.59 | C |
| ATOM | 1691 | NZ | LYS | A | 107 | 35.127 | 18.067 | 15.030 | 1.00 | 61.25 | N |
| ATOM | 1695 | C | LYS | A | 107 | 35.489 | 10.578 | 15.466 | 1.00 | 54.50 | C |
| ATOM | 1696 | O | LYS | A | 107 | 34.661 | 10.520 | 14.550 | 1.00 | 60.65 | O |
| ATOM | 1697 | N | PHE | A | 108 | 36.158 | 9.526 | 15.917 | 1.00 | 48.69 | N |
| ATOM | 1699 | CA | PHE | A | 108 | 35.848 | 8.201 | 15.414 | 1.00 | 47.47 | C |
| ATOM | 1701 | CB | PHE | A | 108 | 37.114 | 7.485 | 14.985 | 1.00 | 46.41 | C |
| ATOM | 1704 | CG | PHE | A | 108 | 36.892 | 6.087 | 14.554 | 1.00 | 44.05 | C |
| ATOM | 1705 | CD1 | PHE | A | 108 | 36.267 | 5.810 | 13.372 | 1.00 | 33.79 | C |
| ATOM | 1707 | CE1 | PHE | A | 108 | 36.073 | 4.549 | 12.988 | 1.00 | 37.34 | C |
| ATOM | 1709 | CZ | PHE | A | 108 | 36.479 | 3.522 | 13.778 | 1.00 | 41.83 | C |
| ATOM | 1711 | CE2 | PHE | A | 108 | 37.111 | 3.773 | 14.959 | 1.00 | 45.59 | C |
| ATOM | 1713 | CD2 | PHE | A | 108 | 37.321 | 5.043 | 15.341 | 1.00 | 45.93 | C |
| ATOM | 1715 | C | PHE | A | 108 | 35.068 | 7.468 | 16.508 | 1.00 | 49.52 | C |
| ATOM | 1716 | O | PHE | A | 108 | 33.900 | 7.133 | 16.347 | 1.00 | 55.52 | O |
| ATOM | 1717 | N | PHE | A | 109 | 35.684 | 7.241 | 17.643 | 1.00 | 49.90 | N |
| ATOM | 1719 | CA | PHE | A | 109 | 34.961 | 6.631 | 18.732 | 1.00 | 51.23 | C |
| ATOM | 1721 | CB | PHE | A | 109 | 35.880 | 6.391 | 19.906 | 1.00 | 53.93 | C |
| ATOM | 1724 | CG | PHE | A | 109 | 36.954 | 5.422 | 19.634 | 1.00 | 57.15 | C |
| ATOM | 1725 | CD1 | PHE | A | 109 | 38.276 | 5.816 | 19.684 | 1.00 | 59.98 | C |
| ATOM | 1727 | CE1 | PHE | A | 109 | 39.280 | 4.917 | 19.436 | 1.00 | 63.80 | C |
| ATOM | 1729 | CZ | PHE | A | 109 | 38.970 | 3.604 | 19.137 | 1.00 | 66.86 | C |
| ATOM | 1731 | CE2 | PHE | A | 109 | 37.643 | 3.200 | 19.088 | 1.00 | 66.40 | C |
| ATOM | 1733 | CD2 | PHE | A | 109 | 36.647 | 4.114 | 19.336 | 1.00 | 61.41 | C |
| ATOM | 1735 | C | PHE | A | 109 | 33.892 | 7.583 | 19.198 | 1.00 | 52.57 | C |
| ATOM | 1736 | O | PHE | A | 109 | 34.087 | 8.778 | 19.130 | 1.00 | 50.58 | O |
| ATOM | 1737 | N | ILE | A | 110 | 32.780 | 7.025 | 19.703 | 1.00 | 57.48 | N |
| ATOM | 1739 | CA | ILE | A | 110 | 31.592 | 7.769 | 20.162 | 1.00 | 51.27 | C |
| ATOM | 1741 | CB | ILE | A | 110 | 30.420 | 7.363 | 19.310 | 1.00 | 49.34 | C |
| ATOM | 1743 | CG1 | ILE | A | 110 | 30.506 | 8.107 | 17.992 | 1.00 | 54.03 | C |
| ATOM | 1746 | CD1 | ILE | A | 110 | 29.249 | 8.085 | 17.198 | 1.00 | 53.87 | C |
| ATOM | 1750 | CG2 | ILE | A | 110 | 29.129 | 7.681 | 19.985 | 1.00 | 54.77 | C |
| ATOM | 1754 | C | ILE | A | 110 | 31.281 | 7.492 | 21.627 | 1.00 | 49.04 | C |
| ATOM | 1755 | O | ILE | A | 110 | 31.108 | 6.355 | 22.046 | 1.00 | 47.53 | O |
| ATOM | 1756 | N | GLN | A | 111 | 31.195 | 8.544 | 22.415 | 1.00 | 53.16 | N |
| ATOM | 1758 | CA | GLN | A | 111 | 31.043 | 8.356 | 23.843 | 1.00 | 56.60 | C |
| ATOM | 1760 | CB | GLN | A | 111 | 30.953 | 9.694 | 24.548 | 1.00 | 58.84 | C |
| ATOM | 1763 | CG | GLN | A | 111 | 30.746 | 9.520 | 26.046 | 1.00 | 62.67 | C |
| ATOM | 1766 | CD | GLN | A | 111 | 31.078 | 10.773 | 26.833 | 1.00 | 66.32 | C |
| ATOM | 1767 | OE1 | GLN | A | 111 | 30.451 | 11.816 | 26.635 | 1.00 | 68.85 | O |
| ATOM | 1768 | NE2 | GLN | A | 111 | 32.068 | 10.677 | 27.724 | 1.00 | 70.23 | N |
| ATOM | 1771 | C | GLN | A | 111 | 29.816 | 7.552 | 24.202 | 1.00 | 57.17 | C |
| ATOM | 1772 | O | GLN | A | 111 | 28.701 | 7.829 | 23.730 | 1.00 | 64.12 | O |
| ATOM | 1773 | N | GLY | A | 112 | 30.012 | 6.560 | 25.043 | 1.00 | 54.22 | N |
| ATOM | 1775 | CA | GLY | A | 112 | 28.893 | 5.803 | 25.547 | 1.00 | 56.70 | C |
| ATOM | 1778 | C | GLY | A | 112 | 28.279 | 4.937 | 24.486 | 1.00 | 58.44 | C |
| ATOM | 1779 | O | GLY | A | 112 | 27.109 | 4.561 | 24.538 | 1.00 | 61.06 | O |
| ATOM | 1780 | N | SER | A | 113 | 29.083 | 4.599 | 23.501 | 1.00 | 61.23 | N |
| ATOM | 1782 | CA | SER | A | 113 | 28.603 | 3.762 | 22.424 | 1.00 | 58.94 | C |
| ATOM | 1784 | CB | SER | A | 113 | 29.408 | 4.080 | 21.174 | 1.00 | 60.39 | C |
| ATOM | 1787 | OG | SER | A | 113 | 30.773 | 4.180 | 21.514 | 1.00 | 59.53 | O |
| ATOM | 1789 | C | SER | A | 113 | 28.687 | 2.272 | 22.768 | 1.00 | 54.01 | C |
| ATOM | 1790 | O | SER | A | 113 | 27.891 | 1.528 | 22.279 | 1.00 | 53.79 | O |
| ATOM | 1791 | N | ASN | A | 114 | 29.642 | 1.879 | 23.613 | 1.00 | 48.58 | N |
| ATOM | 1793 | CA | ASN | A | 114 | 29.882 | 0.505 | 24.026 | 1.00 | 47.72 | C |
| ATOM | 1795 | CB | ASN | A | 114 | 28.660 | −0.222 | 24.481 | 1.00 | 52.15 | C |
| ATOM | 1798 | CG | ASN | A | 114 | 29.015 | −1.423 | 25.395 | 1.00 | 61.51 | C |
| ATOM | 1799 | OD1 | ASN | A | 114 | 29.436 | −1.259 | 26.544 | 1.00 | 64.39 | O |
| ATOM | 1800 | ND2 | ASN | A | 114 | 28.867 | −2.632 | 24.867 | 1.00 | 65.83 | N |
| ATOM | 1803 | C | ASN | A | 114 | 30.664 | −0.364 | 23.028 | 1.00 | 50.29 | C |
| ATOM | 1804 | O | ASN | A | 114 | 30.981 | −1.544 | 23.314 | 1.00 | 51.67 | O |
| ATOM | 1805 | N | TRP | A | 115 | 31.014 | 0.204 | 21.871 | 1.00 | 48.89 | N |
| ATOM | 1807 | CA | TRP | A | 115 | 31.822 | −0.536 | 20.929 | 1.00 | 46.17 | C |
| ATOM | 1809 | CB | TRP | A | 115 | 31.244 | −0.530 | 19.518 | 1.00 | 42.77 | C |
| ATOM | 1812 | CG | TRP | A | 115 | 30.907 | 0.748 | 18.828 | 1.00 | 39.20 | C |
| ATOM | 1813 | CD1 | TRP | A | 115 | 29.722 | 1.338 | 18.787 | 1.00 | 35.62 | C |
| ATOM | 1815 | NE1 | TRP | A | 115 | 29.782 | 2.481 | 18.028 | 1.00 | 36.54 | N |
| ATOM | 1817 | CE2 | TRP | A | 115 | 31.055 | 2.631 | 17.575 | 1.00 | 33.32 | C |
| ATOM | 1818 | CD2 | TRP | A | 115 | 31.783 | 1.553 | 18.053 | 1.00 | 36.65 | C |
| ATOM | 1819 | CE3 | TRP | A | 115 | 33.132 | 1.462 | 17.723 | 1.00 | 37.81 | C |
| ATOM | 1821 | CZ3 | TRP | A | 115 | 33.681 | 2.419 | 16.946 | 1.00 | 32.70 | C |
| ATOM | 1823 | CH2 | TRP | A | 115 | 32.924 | 3.479 | 16.485 | 1.00 | 43.43 | C |
| ATOM | 1825 | CZ2 | TRP | A | 115 | 31.607 | 3.604 | 16.791 | 1.00 | 40.37 | C |
| ATOM | 1827 | C | TRP | A | 115 | 33.277 | −0.054 | 21.024 | 1.00 | 51.28 | C |
| ATOM | 1828 | O | TRP | A | 115 | 33.541 | 0.928 | 21.733 | 1.00 | 54.94 | O |
| ATOM | 1829 | N | GLU | A | 116 | 34.204 | −0.729 | 20.356 | 1.00 | 48.07 | N |
| ATOM | 1831 | CA | GLU | A | 116 | 35.607 | −0.389 | 20.499 | 1.00 | 52.18 | C |
| ATOM | 1833 | CB | GLU | A | 116 | 36.192 | −1.236 | 21.600 | 1.00 | 54.65 | C |
| ATOM | 1836 | CG | GLU | A | 116 | 35.916 | −0.715 | 22.996 | 1.00 | 57.18 | C |
| ATOM | 1839 | CD | GLU | A | 116 | 34.979 | −1.591 | 23.806 | 1.00 | 59.55 | C |
| ATOM | 1840 | OE1 | GLU | A | 116 | 34.289 | −1.014 | 24.701 | 1.00 | 63.96 | O |
| ATOM | 1841 | OE2 | GLU | A | 116 | 34.941 | −2.830 | 23.558 | 1.00 | 53.25 | O |
| ATOM | 1842 | C | GLU | A | 116 | 36.444 | −0.607 | 19.218 | 1.00 | 52.81 | C |
| ATOM | 1843 | O | GLU | A | 116 | 37.686 | −0.501 | 19.227 | 1.00 | 46.45 | O |
| ATOM | 1844 | N | GLY | A | 117 | 35.754 | −0.885 | 18.119 | 1.00 | 48.92 | N |
| ATOM | 1846 | CA | GLY | A | 117 | 36.435 | −1.222 | 16.902 | 1.00 | 47.29 | C |
| ATOM | 1849 | C | GLY | A | 117 | 35.469 | −1.364 | 15.750 | 1.00 | 47.08 | C |
| ATOM | 1850 | O | GLY | A | 117 | 34.257 | −1.112 | 15.798 | 1.00 | 38.00 | O |
| ATOM | 1851 | N | ILE | A | 118 | 35.999 | −1.781 | 14.641 | 1.00 | 43.80 | N |
| ATOM | 1853 | CA | ILE | A | 118 | 35.113 | −1.752 | 13.533 | 1.00 | 39.64 | C |
| ATOM | 1855 | CB | ILE | A | 118 | 35.323 | −0.489 | 12.871 | 1.00 | 33.14 | C |
| ATOM | 1857 | CG1 | ILE | A | 118 | 34.441 | −0.351 | 11.673 | 1.00 | 38.51 | C |
| ATOM | 1860 | CD1 | ILE | A | 118 | 34.304 | 1.118 | 11.253 | 1.00 | 38.06 | C |
| ATOM | 1864 | CG2 | ILE | A | 118 | 36.669 | −0.442 | 12.386 | 1.00 | 41.01 | C |
| ATOM | 1868 | C | ILE | A | 118 | 35.465 | −2.905 | 12.666 | 1.00 | 39.08 | C |
| ATOM | 1869 | O | ILE | A | 118 | 36.608 | −3.210 | 12.482 | 1.00 | 41.16 | O |
| ATOM | 1870 | N | LEU | A | 119 | 34.445 | −3.566 | 12.175 | 1.00 | 39.01 | N |
| ATOM | 1872 | CA | LEU | A | 119 | 34.628 | −4.621 | 11.269 | 1.00 | 38.51 | C |
| ATOM | 1874 | CB | LEU | A | 119 | 33.759 | −5.772 | 11.693 | 1.00 | 39.97 | C |
| ATOM | 1877 | CG | LEU | A | 119 | 33.969 | −6.973 | 10.812 | 1.00 | 40.38 | C |
| ATOM | 1879 | CD1 | LEU | A | 119 | 35.433 | −7.445 | 10.870 | 1.00 | 37.17 | C |
| ATOM | 1883 | CD2 | LEU | A | 119 | 33.036 | −8.028 | 11.273 | 1.00 | 42.73 | C |
| ATOM | 1887 | C | LEU | A | 119 | 34.151 | −4.097 | 9.953 | 1.00 | 38.40 | C |
| ATOM | 1888 | O | LEU | A | 119 | 32.965 | −3.923 | 9.771 | 1.00 | 43.72 | O |
| ATOM | 1889 | N | GLY | A | 120 | 35.059 | −3.794 | 9.039 | 1.00 | 40.26 | N |
| ATOM | 1891 | CA | GLY | A | 120 | 34.655 | −3.388 | 7.696 | 1.00 | 37.27 | C |
| ATOM | 1894 | C | GLY | A | 120 | 34.278 | −4.619 | 6.920 | 1.00 | 35.23 | C |
| ATOM | 1895 | O | GLY | A | 120 | 35.044 | −5.538 | 6.888 | 1.00 | 41.95 | O |
| ATOM | 1896 | N | LEU | A | 121 | 33.116 | −4.697 | 6.293 | 1.00 | 40.05 | N |
| ATOM | 1898 | CA | LEU | A | 121 | 32.866 | −5.891 | 5.473 | 1.00 | 34.58 | C |
| ATOM | 1900 | CB | LEU | A | 121 | 31.626 | −6.622 | 5.917 | 1.00 | 35.45 | C |
| ATOM | 1903 | CG | LEU | A | 121 | 31.436 | −6.831 | 7.393 | 1.00 | 31.35 | C |
| ATOM | 1905 | CD1 | LEU | A | 121 | 29.950 | −6.863 | 7.687 | 1.00 | 28.15 | C |
| ATOM | 1909 | CD2 | LEU | A | 121 | 32.124 | −8.105 | 7.719 | 1.00 | 28.77 | C |
| ATOM | 1913 | C | LEU | A | 121 | 32.704 | −5.635 | 3.984 | 1.00 | 37.29 | C |
| ATOM | 1914 | O | LEU | A | 121 | 32.158 | −6.483 | 3.267 | 1.00 | 42.02 | O |
| ATOM | 1915 | N | ALA | A | 122 | 33.145 | −4.505 | 3.469 | 1.00 | 36.05 | N |
| ATOM | 1917 | CA | ALA | A | 122 | 33.084 | −4.404 | 2.029 | 1.00 | 39.37 | C |
| ATOM | 1919 | CB | ALA | A | 122 | 32.974 | −3.039 | 1.593 | 1.00 | 40.38 | C |
| ATOM | 1923 | C | ALA | A | 122 | 34.289 | −5.090 | 1.398 | 1.00 | 41.97 | C |
| ATOM | 1924 | O | ALA | A | 122 | 35.020 | −5.841 | 2.046 | 1.00 | 40.99 | O |
| ATOM | 1925 | N | TYR | A | 123 | 34.486 | −4.796 | 0.124 | 1.00 | 43.02 | N |
| ATOM | 1927 | CA | TYR | A | 123 | 35.423 | −5.522 | −0.690 | 1.00 | 40.35 | C |
| ATOM | 1929 | CB | TYR | A | 123 | 34.904 | −5.453 | −2.091 | 1.00 | 38.74 | C |
| ATOM | 1932 | CG | TYR | A | 123 | 33.687 | −6.306 | −2.361 | 1.00 | 41.64 | C |
| ATOM | 1933 | CD1 | TYR | A | 123 | 32.453 | −5.755 | −2.557 | 1.00 | 39.22 | C |
| ATOM | 1935 | CE1 | TYR | A | 123 | 31.352 | −6.564 | −2.824 | 1.00 | 34.66 | C |
| ATOM | 1937 | CZ | TYR | A | 123 | 31.487 | −7.899 | −2.896 | 1.00 | 36.31 | C |
| ATOM | 1938 | OH | TYR | A | 123 | 30.392 | −8.724 | −3.167 | 1.00 | 43.22 | O |
| ATOM | 1940 | CE2 | TYR | A | 123 | 32.711 | −8.459 | −2.706 | 1.00 | 43.43 | C |
| ATOM | 1942 | CD2 | TYR | A | 123 | 33.788 | −7.686 | −2.443 | 1.00 | 43.16 | C |
| ATOM | 1944 | C | TYR | A | 123 | 36.758 | −4.856 | −0.586 | 1.00 | 43.96 | C |
| ATOM | 1945 | O | TYR | A | 123 | 36.831 | −3.746 | −0.094 | 1.00 | 43.08 | O |
| ATOM | 1946 | N | ALA | A | 124 | 37.810 | −5.509 | −1.063 | 1.00 | 46.53 | N |
| ATOM | 1948 | CA | ALA | A | 124 | 39.194 | −5.021 | −0.894 | 1.00 | 45.27 | C |
| ATOM | 1950 | CB | ALA | A | 124 | 40.163 | −6.124 | −1.311 | 1.00 | 45.24 | C |
| ATOM | 1954 | C | ALA | A | 124 | 39.597 | −3.728 | −1.616 | 1.00 | 47.88 | C |
| ATOM | 1955 | O | ALA | A | 124 | 40.511 | −2.999 | −1.159 | 1.00 | 47.35 | O |
| ATOM | 1956 | N | GLU | A | 125 | 38.950 | −3.438 | −2.735 | 1.00 | 46.72 | N |
| ATOM | 1958 | CA | GLU | A | 125 | 39.399 | −2.342 | −3.574 | 1.00 | 46.92 | C |
| ATOM | 1960 | CB | GLU | A | 125 | 38.461 | −2.169 | −4.774 | 1.00 | 49.10 | C |
| ATOM | 1963 | CG | GLU | A | 125 | 38.608 | −0.831 | −5.492 | 1.00 | 55.04 | C |
| ATOM | 1966 | CD | GLU | A | 125 | 37.786 | −0.739 | −6.766 | 1.00 | 58.92 | C |
| ATOM | 1967 | OE1 | GLU | A | 125 | 37.095 | −1.730 | −7.135 | 1.00 | 59.91 | O |
| ATOM | 1968 | OE2 | GLU | A | 125 | 37.819 | 0.355 | −7.409 | 1.00 | 68.22 | O |
| ATOM | 1969 | C | GLU | A | 125 | 39.511 | −1.074 | −2.753 | 1.00 | 49.28 | C |
| ATOM | 1970 | O | GLU | A | 125 | 40.486 | −0.298 | −2.858 | 1.00 | 51.06 | O |
| ATOM | 1971 | N | ILE | A | 126 | 38.521 | −0.843 | −1.908 | 1.00 | 46.11 | N |
| ATOM | 1973 | CA | ILE | A | 126 | 38.561 | 0.376 | −1.157 | 1.00 | 43.32 | C |
| ATOM | 1975 | CB | ILE | A | 126 | 37.142 | 0.953 | −0.961 | 1.00 | 47.04 | C |
| ATOM | 1977 | CG1 | ILE | A | 126 | 36.254 | 0.022 | −0.122 | 1.00 | 44.04 | C |
| ATOM | 1980 | CD1 | ILE | A | 126 | 34.965 | 0.642 | 0.383 | 1.00 | 50.45 | C |
| ATOM | 1984 | CG2 | ILE | A | 126 | 36.539 | 1.274 | −2.351 | 1.00 | 46.90 | C |
| ATOM | 1988 | C | ILE | A | 126 | 39.288 | 0.228 | 0.142 | 1.00 | 44.66 | C |
| ATOM | 1989 | O | ILE | A | 126 | 39.146 | 1.085 | 1.007 | 1.00 | 48.22 | O |
| ATOM | 1990 | N | ALA | A | 127 | 40.093 | −0.808 | 0.310 | 1.00 | 44.81 | N |
| ATOM | 1992 | CA | ALA | A | 127 | 40.788 | −0.914 | 1.580 | 1.00 | 48.39 | C |
| ATOM | 1994 | CB | ALA | A | 127 | 40.982 | −2.321 | 1.937 | 1.00 | 50.81 | C |
| ATOM | 1998 | C | ALA | A | 127 | 42.119 | −0.200 | 1.613 | 1.00 | 52.76 | C |
| ATOM | 1999 | O | ALA | A | 127 | 42.699 | 0.145 | 0.574 | 1.00 | 59.40 | O |
| ATOM | 2000 | N | ARG | A | 128 | 42.591 | 0.010 | 2.838 | 1.00 | 51.36 | N |
| ATOM | 2002 | CA | ARG | A | 128 | 43.783 | 0.715 | 3.064 | 1.00 | 51.80 | C |
| ATOM | 2004 | CB | ARG | A | 128 | 43.430 | 1.900 | 3.897 | 1.00 | 56.05 | C |
| ATOM | 2007 | CG | ARG | A | 128 | 42.275 | 2.626 | 3.401 | 1.00 | 59.64 | C |
| ATOM | 2010 | CD | ARG | A | 128 | 42.295 | 2.934 | 1.917 | 1.00 | 69.11 | C |
| ATOM | 2013 | NE | ARG | A | 128 | 41.482 | 4.108 | 1.663 | 1.00 | 79.76 | N |
| ATOM | 2015 | CZ | ARG | A | 128 | 41.839 | 5.318 | 2.018 | 1.00 | 81.91 | C |
| ATOM | 2016 | NH1 | ARG | A | 128 | 43.001 | 5.508 | 2.630 | 1.00 | 80.28 | N |
| ATOM | 2019 | NH2 | ARG | A | 128 | 41.039 | 6.335 | 1.759 | 1.00 | 87.99 | N |
| ATOM | 2022 | C | ARG | A | 128 | 44.831 | −0.135 | 3.828 | 1.00 | 51.97 | C |
| ATOM | 2023 | O | ARG | A | 128 | 44.461 | −0.987 | 4.689 | 1.00 | 49.70 | O |
| ATOM | 2024 | N | PRO | A | 129 | 46.129 | 0.076 | 3.544 | 1.00 | 44.07 | N |
| ATOM | 2025 | CA | PRO | A | 129 | 46.636 | 1.004 | 2.539 | 1.00 | 39.23 | C |
| ATOM | 2027 | CB | PRO | A | 129 | 48.105 | 0.812 | 2.595 | 1.00 | 39.26 | C |
| ATOM | 2030 | CG | PRO | A | 129 | 48.386 | −0.019 | 3.710 | 1.00 | 44.63 | C |
| ATOM | 2033 | CD | PRO | A | 129 | 47.196 | −0.642 | 4.242 | 1.00 | 43.20 | C |
| ATOM | 2036 | C | PRO | A | 129 | 46.254 | 0.674 | 1.113 | 1.00 | 45.48 | C |
| ATOM | 2037 | O | PRO | A | 129 | 46.153 | 1.658 | 0.336 | 1.00 | 46.05 | O |
| ATOM | 2038 | N | ASP | A | 130 | 46.101 | −0.637 | 0.783 | 1.00 | 46.52 | N |
| ATOM | 2040 | CA | ASP | A | 130 | 45.574 | −1.009 | −0.520 | 1.00 | 47.07 | C |
| ATOM | 2042 | CB | ASP | A | 130 | 46.563 | −0.676 | −1.653 | 1.00 | 52.49 | C |
| ATOM | 2045 | CG | ASP | A | 130 | 47.874 | −1.365 | −1.520 | 1.00 | 58.99 | C |
| ATOM | 2046 | OD1 | ASP | A | 130 | 48.849 | −0.917 | −2.187 | 1.00 | 66.76 | O |
| ATOM | 2047 | OD2 | ASP | A | 130 | 48.023 | −2.366 | −0.794 | 1.00 | 64.97 | O |
| ATOM | 2048 | C | ASP | A | 130 | 44.937 | −2.355 | −0.652 | 1.00 | 44.59 | C |
| ATOM | 2049 | O | ASP | A | 130 | 44.877 | −3.167 | 0.272 | 1.00 | 50.22 | O |
| ATOM | 2050 | N | ASP | A | 131 | 44.443 | −2.606 | −1.834 | 1.00 | 41.96 | N |
| ATOM | 2052 | CA | ASP | A | 131 | 43.679 | −3.802 | −2.024 | 1.00 | 45.38 | C |
| ATOM | 2054 | CB | ASP | A | 131 | 43.076 | −3.828 | −3.427 | 1.00 | 45.37 | C |
| ATOM | 2057 | CG | ASP | A | 131 | 44.106 | −4.043 | −4.476 | 1.00 | 54.14 | C |
| ATOM | 2058 | OD1 | ASP | A | 131 | 45.130 | −4.692 | −4.143 | 1.00 | 60.53 | O |
| ATOM | 2059 | OD2 | ASP | A | 131 | 43.983 | −3.593 | −5.648 | 1.00 | 58.51 | O |
| ATOM | 2060 | C | ASP | A | 131 | 44.437 | −5.085 | −1.770 | 1.00 | 41.04 | C |
| ATOM | 2061 | O | ASP | A | 131 | 43.912 | −6.123 | −2.065 | 1.00 | 49.55 | O |
| ATOM | 2062 | N | SER | A | 132 | 45.645 | −5.050 | −1.247 | 1.00 | 39.75 | N |
| ATOM | 2064 | CA | SER | A | 132 | 46.409 | −6.304 | −1.002 | 1.00 | 45.06 | C |
| ATOM | 2066 | CB | SER | A | 132 | 47.788 | −6.174 | −1.605 | 1.00 | 43.25 | C |
| ATOM | 2069 | OG | SER | A | 132 | 48.375 | −4.995 | −1.060 | 1.00 | 53.72 | O |
| ATOM | 2071 | C | SER | A | 132 | 46.582 | −6.591 | 0.489 | 1.00 | 41.26 | C |
| ATOM | 2072 | O | SER | A | 132 | 47.588 | −7.115 | 0.938 | 1.00 | 52.36 | O |
| ATOM | 2073 | N | LEU | A | 133 | 45.584 | −6.162 | 1.242 | 1.00 | 43.42 | N |
| ATOM | 2075 | CA | LEU | A | 133 | 45.558 | −6.239 | 2.687 | 1.00 | 44.69 | C |
| ATOM | 2077 | CB | LEU | A | 133 | 45.773 | −4.923 | 3.419 | 1.00 | 41.73 | C |
| ATOM | 2080 | CG | LEU | A | 133 | 45.518 | −5.228 | 4.908 | 1.00 | 41.79 | C |
| ATOM | 2082 | CD1 | LEU | A | 133 | 46.832 | −5.498 | 5.559 | 1.00 | 39.80 | C |
| ATOM | 2086 | CD2 | LEU | A | 133 | 44.771 | −4.164 | 5.716 | 1.00 | 44.00 | C |
| ATOM | 2090 | C | LEU | A | 133 | 44.166 | −6.677 | 2.929 | 1.00 | 47.10 | C |
| ATOM | 2091 | O | LEU | A | 133 | 43.330 | −5.956 | 3.439 | 1.00 | 51.69 | O |
| ATOM | 2092 | N | GLU | A | 134 | 43.977 | −7.906 | 2.510 | 1.00 | 51.73 | N |
| ATOM | 2094 | CA | GLU | A | 134 | 42.777 | −8.725 | 2.630 | 1.00 | 48.06 | C |
| ATOM | 2096 | CB | GLU | A | 134 | 43.241 | −10.178 | 2.777 | 1.00 | 47.21 | C |
| ATOM | 2099 | CG | GLU | A | 134 | 42.169 | −11.212 | 3.013 | 1.00 | 52.87 | C |
| ATOM | 2102 | CD | GLU | A | 134 | 42.689 | −12.623 | 2.755 | 1.00 | 56.68 | C |
| ATOM | 2103 | OE1 | GLU | A | 134 | 42.980 | −12.874 | 1.569 | 1.00 | 55.42 | O |
| ATOM | 2104 | OE2 | GLU | A | 134 | 42.817 | −13.448 | 3.709 | 1.00 | 55.93 | O |
| ATOM | 2105 | C | GLU | A | 134 | 41.837 | −8.500 | 3.783 | 1.00 | 46.74 | C |
| ATOM | 2106 | O | GLU | A | 134 | 42.237 | −8.575 | 4.940 | 1.00 | 43.54 | O |
| ATOM | 2107 | N | PRO | A | 135 | 40.576 | −8.279 | 3.446 | 1.00 | 40.56 | N |
| ATOM | 2108 | CA | PRO | A | 135 | 39.531 | −8.099 | 4.437 | 1.00 | 37.41 | C |
| ATOM | 2110 | CB | PRO | A | 135 | 38.355 | −7.629 | 3.597 | 1.00 | 37.44 | C |
| ATOM | 2113 | CG | PRO | A | 135 | 38.853 | −7.363 | 2.237 | 1.00 | 40.45 | C |
| ATOM | 2116 | CD | PRO | A | 135 | 40.071 | −8.171 | 2.068 | 1.00 | 42.71 | C |
| ATOM | 2119 | C | PRO | A | 135 | 39.174 | −9.353 | 5.269 | 1.00 | 36.44 | C |
| ATOM | 2120 | O | PRO | A | 135 | 39.366 | −10.450 | 4.767 | 1.00 | 34.69 | O |
| ATOM | 2121 | N | PHE | A | 136 | 38.668 | −9.163 | 6.505 | 1.00 | 35.67 | N |
| ATOM | 2123 | CA | PHE | A | 136 | 38.233 | −10.241 | 7.427 | 1.00 | 31.72 | C |
| ATOM | 2125 | CB | PHE | A | 136 | 37.463 | −9.676 | 8.625 | 1.00 | 27.91 | C |
| ATOM | 2128 | CG | PHE | A | 136 | 36.835 | −10.727 | 9.517 | 1.00 | 28.87 | C |
| ATOM | 2129 | CD1 | PHE | A | 136 | 37.537 | −11.301 | 10.550 | 1.00 | 32.96 | C |
| ATOM | 2131 | CE1 | PHE | A | 136 | 36.966 | −12.267 | 11.382 | 1.00 | 33.11 | C |
| ATOM | 2133 | CZ | PHE | A | 136 | 35.694 | −12.663 | 11.195 | 1.00 | 33.91 | C |
| ATOM | 2135 | CE2 | PHE | A | 136 | 34.968 | −12.111 | 10.175 | 1.00 | 36.07 | C |
| ATOM | 2137 | CD2 | PHE | A | 136 | 35.544 | −11.137 | 9.333 | 1.00 | 37.92 | C |
| ATOM | 2139 | C | PHE | A | 136 | 37.332 | −11.218 | 6.753 | 1.00 | 35.77 | C |
| ATOM | 2140 | O | PHE | A | 136 | 37.649 | −12.376 | 6.668 | 1.00 | 45.44 | O |
| ATOM | 2141 | N | PHE | A | 137 | 36.194 | −10.778 | 6.261 | 1.00 | 40.56 | N |
| ATOM | 2143 | CA | PHE | A | 137 | 35.282 | −11.754 | 5.677 | 1.00 | 43.42 | C |
| ATOM | 2145 | CB | PHE | A | 137 | 34.008 | −11.132 | 5.014 | 1.00 | 43.68 | C |
| ATOM | 2148 | CG | PHE | A | 137 | 32.841 | −12.067 | 5.028 | 1.00 | 41.41 | C |
| ATOM | 2149 | CD1 | PHE | A | 137 | 32.094 | −12.231 | 6.160 | 1.00 | 38.90 | C |
| ATOM | 2151 | CE1 | PHE | A | 137 | 31.051 | −13.107 | 6.179 | 1.00 | 36.47 | C |
| ATOM | 2153 | CZ | PHE | A | 137 | 30.751 | −13.824 | 5.087 | 1.00 | 36.88 | C |
| ATOM | 2155 | CE2 | PHE | A | 137 | 31.470 | −13.682 | 3.958 | 1.00 | 39.68 | C |
| ATOM | 2157 | CD2 | PHE | A | 137 | 32.516 | −12.809 | 3.922 | 1.00 | 43.99 | C |
| ATOM | 2159 | C | PHE | A | 137 | 36.106 | −12.571 | 4.704 | 1.00 | 43.89 | C |
| ATOM | 2160 | O | PHE | A | 137 | 36.159 | −13.774 | 4.828 | 1.00 | 41.80 | O |
| ATOM | 2161 | N | ASP | A | 138 | 36.778 | −11.904 | 3.762 | 1.00 | 47.91 | N |
| ATOM | 2163 | CA | ASP | A | 138 | 37.627 | −12.581 | 2.776 | 1.00 | 47.51 | C |
| ATOM | 2165 | CB | ASP | A | 138 | 38.618 | −11.632 | 2.142 | 1.00 | 51.24 | C |
| ATOM | 2168 | CG | ASP | A | 138 | 38.290 | −11.344 | 0.725 | 1.00 | 54.56 | C |
| ATOM | 2169 | OD1 | ASP | A | 138 | 38.836 | −12.027 | −0.179 | 1.00 | 66.25 | O |
| ATOM | 2170 | OD2 | ASP | A | 138 | 37.492 | −10.434 | 0.419 | 1.00 | 57.65 | O |
| ATOM | 2171 | C | ASP | A | 138 | 38.436 | −13.684 | 3.394 | 1.00 | 47.78 | C |
| ATOM | 2172 | O | ASP | A | 138 | 38.505 | −14.788 | 2.886 | 1.00 | 57.75 | O |
| ATOM | 2173 | N | SER | A | 139 | 39.097 | −13.385 | 4.482 | 1.00 | 46.16 | N |
| ATOM | 2175 | CA | SER | A | 139 | 39.868 | −14.405 | 5.145 | 1.00 | 42.31 | C |
| ATOM | 2177 | CB | SER | A | 139 | 40.721 | −13.740 | 6.204 | 1.00 | 42.45 | C |
| ATOM | 2180 | OG | SER | A | 139 | 41.538 | −12.754 | 5.615 | 1.00 | 35.20 | O |
| ATOM | 2182 | C | SER | A | 139 | 38.967 | −15.421 | 5.797 | 1.00 | 43.01 | C |
| ATOM | 2183 | O | SER | A | 139 | 39.302 | −16.570 | 5.891 | 1.00 | 42.82 | O |
| ATOM | 2184 | N | LEU | A | 140 | 37.801 | −15.016 | 6.279 | 1.00 | 50.41 | N |
| ATOM | 2186 | CA | LEU | A | 140 | 36.939 | −15.973 | 7.006 | 1.00 | 46.59 | C |
| ATOM | 2188 | CB | LEU | A | 140 | 35.697 | −15.302 | 7.483 | 1.00 | 47.69 | C |
| ATOM | 2191 | CG | LEU | A | 140 | 34.729 | −16.175 | 8.248 | 1.00 | 54.03 | C |
| ATOM | 2193 | CD1 | LEU | A | 140 | 35.440 | −16.828 | 9.380 | 1.00 | 56.74 | C |
| ATOM | 2197 | CD2 | LEU | A | 140 | 33.562 | −15.327 | 8.755 | 1.00 | 52.27 | C |
| ATOM | 2201 | C | LEU | A | 140 | 36.541 | −17.129 | 6.139 | 1.00 | 48.27 | C |
| ATOM | 2202 | O | LEU | A | 140 | 36.391 | −18.231 | 6.649 | 1.00 | 48.66 | O |
| ATOM | 2203 | N | VAL | A | 141 | 36.363 | −16.868 | 4.833 | 1.00 | 46.25 | N |
| ATOM | 2205 | CA | VAL | A | 141 | 36.054 | −17.909 | 3.873 | 1.00 | 48.65 | C |
| ATOM | 2207 | CB | VAL | A | 141 | 35.281 | −17.376 | 2.644 | 1.00 | 49.70 | C |
| ATOM | 2209 | CG1 | VAL | A | 141 | 35.044 | −18.555 | 1.662 | 1.00 | 50.68 | C |
| ATOM | 2213 | CG2 | VAL | A | 141 | 33.952 | −16.801 | 3.069 | 1.00 | 50.47 | C |
| ATOM | 2217 | C | VAL | A | 141 | 37.251 | −18.777 | 3.388 | 1.00 | 54.04 | C |
| ATOM | 2218 | O | VAL | A | 141 | 37.121 | −19.989 | 3.354 | 1.00 | 62.33 | O |
| ATOM | 2219 | N | LYS | A | 142 | 38.390 | −18.195 | 3.009 | 1.00 | 53.50 | N |
| ATOM | 2221 | CA | LYS | A | 142 | 39.497 | −18.997 | 2.510 | 1.00 | 51.72 | C |
| ATOM | 2223 | CB | LYS | A | 142 | 40.720 | −18.173 | 2.027 | 1.00 | 51.19 | C |
| ATOM | 2226 | CG | LYS | A | 142 | 40.466 | −16.996 | 1.006 | 1.00 | 55.43 | C |
| ATOM | 2229 | CD | LYS | A | 142 | 41.790 | −16.385 | 0.423 | 1.00 | 59.35 | C |
| ATOM | 2232 | CE | LYS | A | 142 | 41.577 | −15.659 | −0.919 | 1.00 | 61.18 | C |
| ATOM | 2235 | NZ | LYS | A | 142 | 40.212 | −15.043 | −0.953 | 1.00 | 69.14 | N |
| ATOM | 2239 | C | LYS | A | 142 | 39.970 | −20.002 | 3.571 | 1.00 | 52.97 | C |
| ATOM | 2240 | O | LYS | A | 142 | 40.643 | −20.968 | 3.211 | 1.00 | 55.66 | O |
| ATOM | 2241 | N | GLN | A | 143 | 39.634 | −19.800 | 4.851 | 1.00 | 47.49 | N |
| ATOM | 2243 | CA | GLN | A | 143 | 40.189 | −20.638 | 5.937 | 1.00 | 47.98 | C |
| ATOM | 2245 | CB | GLN | A | 143 | 40.818 | −19.777 | 7.058 | 1.00 | 44.95 | C |
| ATOM | 2248 | CG | GLN | A | 143 | 42.180 | −19.165 | 6.780 | 1.00 | 45.74 | C |
| ATOM | 2251 | CD | GLN | A | 143 | 42.522 | −17.951 | 7.691 | 1.00 | 52.26 | C |
| ATOM | 2252 | OE1 | GLN | A | 143 | 42.548 | −18.077 | 8.918 | 1.00 | 55.17 | O |
| ATOM | 2253 | NE2 | GLN | A | 143 | 42.787 | −16.783 | 7.081 | 1.00 | 48.24 | N |
| ATOM | 2256 | C | GLN | A | 143 | 39.190 | −21.579 | 6.594 | 1.00 | 52.56 | C |
| ATOM | 2257 | O | GLN | A | 143 | 39.505 | −22.219 | 7.600 | 1.00 | 57.47 | O |
| ATOM | 2258 | N | THR | A | 144 | 37.989 | −21.675 | 6.041 | 1.00 | 59.44 | N |
| ATOM | 2260 | CA | THR | A | 144 | 36.913 | −22.471 | 6.651 | 1.00 | 58.58 | C |
| ATOM | 2262 | CB | THR | A | 144 | 36.225 | −21.737 | 7.797 | 1.00 | 56.28 | C |
| ATOM | 2264 | OG1 | THR | A | 144 | 35.191 | −20.904 | 7.267 | 1.00 | 60.07 | O |
| ATOM | 2266 | CG2 | THR | A | 144 | 37.139 | −20.769 | 8.490 | 1.00 | 59.12 | C |
| ATOM | 2270 | C | THR | A | 144 | 35.875 | −22.756 | 5.581 | 1.00 | 59.57 | C |
| ATOM | 2271 | O | THR | A | 144 | 36.071 | −22.385 | 4.425 | 1.00 | 61.12 | O |
| ATOM | 2272 | N | HIS | A | 145 | 34.772 | −23.401 | 5.946 | 1.00 | 57.96 | N |
| ATOM | 2274 | CA | HIS | A | 145 | 33.786 | −23.769 | 4.930 | 1.00 | 61.46 | C |
| ATOM | 2276 | CB | HIS | A | 145 | 33.315 | −25.229 | 5.146 | 1.00 | 66.92 | C |
| ATOM | 2279 | CG | HIS | A | 145 | 34.393 | −26.252 | 4.917 | 1.00 | 73.97 | C |
| ATOM | 2280 | ND1 | HIS | A | 145 | 35.092 | −26.844 | 5.950 | 1.00 | 78.11 | N |
| ATOM | 2282 | CE1 | HIS | A | 145 | 35.980 | −27.687 | 5.453 | 1.00 | 77.67 | C |
| ATOM | 2284 | NE2 | HIS | A | 145 | 35.882 | −27.665 | 4.134 | 1.00 | 76.27 | N |
| ATOM | 2286 | CD2 | HIS | A | 145 | 34.897 | −26.777 | 3.774 | 1.00 | 75.10 | C |
| ATOM | 2288 | C | HIS | A | 145 | 32.587 | −22.826 | 4.899 | 1.00 | 57.13 | C |
| ATOM | 2289 | O | HIS | A | 145 | 31.592 | −23.086 | 4.214 | 1.00 | 57.76 | O |
| ATOM | 2290 | N | VAL | A | 146 | 32.679 | −21.728 | 5.629 | 1.00 | 52.17 | N |
| ATOM | 2292 | CA | VAL | A | 146 | 31.539 | −20.847 | 5.769 | 1.00 | 47.04 | C |
| ATOM | 2294 | CB | VAL | A | 146 | 31.849 | −19.669 | 6.681 | 1.00 | 48.57 | C |
| ATOM | 2296 | CG1 | VAL | A | 146 | 30.713 | −18.715 | 6.687 | 1.00 | 48.07 | C |
| ATOM | 2300 | CG2 | VAL | A | 146 | 32.122 | −20.147 | 8.110 | 1.00 | 49.78 | C |
| ATOM | 2304 | C | VAL | A | 146 | 31.247 | −20.301 | 4.426 | 1.00 | 42.53 | C |
| ATOM | 2305 | O | VAL | A | 146 | 32.116 | −19.728 | 3.817 | 1.00 | 45.79 | O |
| ATOM | 2306 | N | PRO | A | 147 | 30.029 | −20.472 | 3.950 | 1.00 | 37.44 | N |
| ATOM | 2307 | CA | PRO | A | 147 | 29.613 | −19.931 | 2.649 | 1.00 | 36.28 | C |
| ATOM | 2309 | CB | PRO | A | 147 | 28.139 | −20.275 | 2.599 | 1.00 | 38.81 | C |
| ATOM | 2312 | CG | PRO | A | 147 | 28.062 | −21.485 | 3.451 | 1.00 | 37.97 | C |
| ATOM | 2315 | CD | PRO | A | 147 | 28.953 | −21.213 | 4.609 | 1.00 | 37.03 | C |
| ATOM | 2318 | C | PRO | A | 147 | 29.793 | −18.397 | 2.535 | 1.00 | 38.62 | C |
| ATOM | 2319 | O | PRO | A | 147 | 29.688 | −17.668 | 3.519 | 1.00 | 36.50 | O |
| ATOM | 2320 | N | ASN | A | 148 | 30.042 | −17.926 | 1.318 | 1.00 | 37.03 | N |
| ATOM | 2322 | CA | ASN | A | 148 | 30.370 | −16.536 | 1.041 | 1.00 | 35.69 | C |
| ATOM | 2324 | CB | ASN | A | 148 | 31.113 | −16.480 | −0.280 | 1.00 | 35.18 | C |
| ATOM | 2327 | CG | ASN | A | 148 | 31.791 | −15.208 | −0.460 | 1.00 | 38.29 | C |
| ATOM | 2328 | OD1 | ASN | A | 148 | 31.990 | −14.492 | 0.507 | 1.00 | 48.87 | O |
| ATOM | 2329 | ND2 | ASN | A | 148 | 32.170 | −14.888 | −1.683 | 1.00 | 45.21 | N |
| ATOM | 2332 | C | ASN | A | 148 | 29.184 | −15.584 | 0.950 | 1.00 | 37.44 | C |
| ATOM | 2333 | O | ASN | A | 148 | 28.928 | −14.994 | −0.110 | 1.00 | 34.38 | O |
| ATOM | 2334 | N | LEU | A | 149 | 28.498 | −15.407 | 2.075 | 1.00 | 39.14 | N |
| ATOM | 2336 | CA | LEU | A | 149 | 27.269 | −14.634 | 2.168 | 1.00 | 37.49 | C |
| ATOM | 2338 | CB | LEU | A | 149 | 26.071 | −15.476 | 1.699 | 1.00 | 38.02 | C |
| ATOM | 2341 | CG | LEU | A | 149 | 24.571 | −15.300 | 2.049 | 1.00 | 45.01 | C |
| ATOM | 2343 | CD1 | LEU | A | 149 | 23.843 | −16.146 | 1.018 | 1.00 | 46.47 | C |
| ATOM | 2347 | CD2 | LEU | A | 149 | 24.051 | −15.748 | 3.449 | 1.00 | 46.97 | C |
| ATOM | 2351 | C | LEU | A | 149 | 27.073 | −14.349 | 3.619 | 1.00 | 41.29 | C |
| ATOM | 2352 | O | LEU | A | 149 | 27.374 | −15.200 | 4.445 | 1.00 | 43.07 | O |
| ATOM | 2353 | N | PHE | A | 150 | 26.597 | −13.160 | 3.957 | 1.00 | 43.88 | N |
| ATOM | 2355 | CA | PHE | A | 150 | 26.128 | −12.896 | 5.313 | 1.00 | 37.90 | C |
| ATOM | 2357 | CB | PHE | A | 150 | 27.151 | −12.168 | 6.146 | 1.00 | 36.97 | C |
| ATOM | 2360 | CG | PHE | A | 150 | 27.514 | −10.855 | 5.616 | 1.00 | 38.62 | C |
| ATOM | 2361 | CD1 | PHE | A | 150 | 28.508 | −10.731 | 4.664 | 1.00 | 43.40 | C |
| ATOM | 2363 | CE1 | PHE | A | 150 | 28.849 | −9.516 | 4.157 | 1.00 | 36.52 | C |
| ATOM | 2365 | CZ | PHE | A | 150 | 28.202 | −8.415 | 4.609 | 1.00 | 39.56 | C |
| ATOM | 2367 | CE2 | PHE | A | 150 | 27.220 | −8.530 | 5.564 | 1.00 | 32.53 | C |
| ATOM | 2369 | CD2 | PHE | A | 150 | 26.885 | −9.737 | 6.055 | 1.00 | 36.36 | C |
| ATOM | 2371 | C | PHE | A | 150 | 24.854 | −12.097 | 5.122 | 1.00 | 41.04 | C |
| ATOM | 2372 | O | PHE | A | 150 | 24.632 | −11.537 | 4.054 | 1.00 | 41.82 | O |
| ATOM | 2373 | N | SER | A | 151 | 23.980 | −12.071 | 6.121 | 1.00 | 44.62 | N |
| ATOM | 2375 | CA | SER | A | 151 | 22.760 | −11.287 | 6.018 | 1.00 | 40.62 | C |
| ATOM | 2377 | CB | SER | A | 151 | 21.553 | −12.207 | 5.931 | 1.00 | 43.08 | C |
| ATOM | 2380 | OG | SER | A | 151 | 21.673 | −13.313 | 6.803 | 1.00 | 53.44 | O |
| ATOM | 2382 | C | SER | A | 151 | 22.708 | −10.401 | 7.238 | 1.00 | 39.25 | C |
| ATOM | 2383 | O | SER | A | 151 | 23.371 | −10.749 | 8.190 | 1.00 | 40.13 | O |
| ATOM | 2384 | N | LEU | A | 152 | 21.985 | −9.260 | 7.214 | 1.00 | 38.62 | N |
| ATOM | 2386 | CA | LEU | A | 152 | 21.755 | −8.448 | 8.435 | 1.00 | 36.45 | C |
| ATOM | 2388 | CB | LEU | A | 152 | 22.588 | −7.166 | 8.446 | 1.00 | 35.08 | C |
| ATOM | 2391 | CG | LEU | A | 152 | 24.121 | −7.237 | 8.489 | 1.00 | 29.92 | C |
| ATOM | 2393 | CD1 | LEU | A | 152 | 24.769 | −5.957 | 8.014 | 1.00 | 28.56 | C |
| ATOM | 2397 | CD2 | LEU | A | 152 | 24.536 | −7.467 | 9.812 | 1.00 | 30.11 | C |
| ATOM | 2401 | C | LEU | A | 152 | 20.305 | −8.042 | 8.672 | 1.00 | 37.58 | C |
| ATOM | 2402 | O | LEU | A | 152 | 19.591 | −7.681 | 7.756 | 1.00 | 49.31 | O |
| ATOM | 2403 | N | GLN | A | 153 | 19.877 | −8.069 | 9.925 | 1.00 | 42.17 | N |
| ATOM | 2405 | CA | GLN | A | 153 | 18.544 | −7.609 | 10.332 | 1.00 | 37.31 | C |
| ATOM | 2407 | CB | GLN | A | 153 | 17.636 | −8.817 | 10.719 | 1.00 | 34.23 | C |
| ATOM | 2410 | CG | GLN | A | 153 | 16.211 | −8.478 | 11.173 | 1.00 | 38.27 | C |
| ATOM | 2413 | CD | GLN | A | 153 | 15.562 | −9.519 | 12.109 | 1.00 | 47.39 | C |
| ATOM | 2414 | OE1 | GLN | A | 153 | 16.047 | −9.755 | 13.231 | 1.00 | 58.66 | O |
| ATOM | 2415 | NE2 | GLN | A | 153 | 14.465 | −10.125 | 11.663 | 1.00 | 39.51 | N |
| ATOM | 2418 | C | GLN | A | 153 | 18.798 | −6.689 | 11.532 | 1.00 | 39.67 | C |
| ATOM | 2419 | O | GLN | A | 153 | 18.980 | −7.157 | 12.638 | 1.00 | 47.11 | O |
| ATOM | 2420 | N | LEU | A | 154 | 18.830 | −5.384 | 11.320 | 1.00 | 42.61 | N |
| ATOM | 2422 | CA | LEU | A | 154 | 19.057 | −4.410 | 12.397 | 1.00 | 42.93 | C |
| ATOM | 2424 | CB | LEU | A | 154 | 19.826 | −3.221 | 11.839 | 1.00 | 45.84 | C |
| ATOM | 2427 | CG | LEU | A | 154 | 21.012 | −3.514 | 10.947 | 1.00 | 40.27 | C |
| ATOM | 2429 | CD1 | LEU | A | 154 | 21.452 | −2.253 | 10.295 | 1.00 | 42.77 | C |
| ATOM | 2433 | CD2 | LEU | A | 154 | 22.137 | −4.067 | 11.759 | 1.00 | 42.65 | C |
| ATOM | 2437 | C | LEU | A | 154 | 17.700 | −3.937 | 12.912 | 1.00 | 42.07 | C |
| ATOM | 2438 | O | LEU | A | 154 | 16.804 | −3.804 | 12.136 | 1.00 | 50.21 | O |
| ATOM | 2439 | N | CYS | A | 155 | 17.538 | −3.646 | 14.191 | 1.00 | 45.97 | N |
| ATOM | 2441 | CA | CYS | A | 155 | 16.180 | −3.612 | 14.763 | 1.00 | 51.90 | C |
| ATOM | 2443 | CB | CYS | A | 155 | 15.927 | −4.865 | 15.662 | 1.00 | 51.86 | C |
| ATOM | 2446 | SG | CYS | A | 155 | 15.554 | −6.528 | 14.975 | 1.00 | 51.81 | S |
| ATOM | 2447 | C | CYS | A | 155 | 15.967 | −2.390 | 15.642 | 1.00 | 57.62 | C |
| ATOM | 2448 | O | CYS | A | 155 | 16.114 | −2.476 | 16.878 | 1.00 | 63.11 | O |
| ATOM | 2449 | N | GLY | A | 156 | 15.631 | −1.257 | 15.041 | 1.00 | 58.85 | N |
| ATOM | 2451 | CA | GLY | A | 156 | 15.380 | −0.072 | 15.830 | 1.00 | 62.65 | C |
| ATOM | 2454 | C | GLY | A | 156 | 14.226 | −0.446 | 16.728 | 1.00 | 66.48 | C |
| ATOM | 2455 | O | GLY | A | 156 | 13.447 | −1.309 | 16.342 | 1.00 | 69.64 | O |
| ATOM | 2456 | N | ALA | A | 157 | 14.088 | 0.144 | 17.910 | 1.00 | 69.44 | N |
| ATOM | 2458 | CA | ALA | A | 157 | 12.896 | −0.169 | 18.704 | 1.00 | 72.46 | C |
| ATOM | 2460 | CB | ALA | A | 157 | 13.304 | −0.588 | 20.111 | 1.00 | 75.06 | C |
| ATOM | 2464 | C | ALA | A | 157 | 11.799 | 0.960 | 18.698 | 1.00 | 73.42 | C |
| ATOM | 2465 | O | ALA | A | 157 | 10.749 | 0.888 | 19.359 | 1.00 | 73.68 | O |
| ATOM | 2466 | N | GLY | A | 158 | 12.015 | 2.000 | 17.926 | 1.00 | 74.47 | N |
| ATOM | 2468 | CA | GLY | A | 158 | 11.010 | 3.043 | 17.837 | 1.00 | 78.58 | C |
| ATOM | 2471 | C | GLY | A | 158 | 10.829 | 3.954 | 19.059 | 1.00 | 82.73 | C |
| ATOM | 2472 | O | GLY | A | 158 | 9.947 | 4.825 | 19.033 | 1.00 | 85.96 | O |
| ATOM | 2473 | N | PHE | A | 159 | 11.633 | 3.774 | 20.115 | 1.00 | 83.55 | N |
| ATOM | 2475 | CA | PHE | A | 159 | 11.564 | 4.626 | 21.313 | 1.00 | 85.05 | C |
| ATOM | 2477 | CB | PHE | A | 159 | 10.558 | 4.054 | 22.277 | 1.00 | 85.74 | C |
| ATOM | 2480 | CG | PHE | A | 159 | 10.993 | 2.748 | 22.855 | 1.00 | 82.94 | C |
| ATOM | 2481 | CD1 | PHE | A | 159 | 10.305 | 1.593 | 22.561 | 1.00 | 82.82 | C |
| ATOM | 2483 | CE1 | PHE | A | 159 | 10.717 | 0.373 | 23.096 | 1.00 | 83.66 | C |
| ATOM | 2485 | CZ | PHE | A | 159 | 11.831 | 0.325 | 23.935 | 1.00 | 82.59 | C |
| ATOM | 2487 | CE2 | PHE | A | 159 | 12.519 | 1.491 | 24.228 | 1.00 | 80.31 | C |
| ATOM | 2489 | CD2 | PHE | A | 159 | 12.099 | 2.683 | 23.686 | 1.00 | 79.87 | C |
| ATOM | 2491 | C | PHE | A | 159 | 12.910 | 4.671 | 22.044 | 1.00 | 87.61 | C |
| ATOM | 2492 | O | PHE | A | 159 | 13.494 | 3.624 | 22.302 | 1.00 | 87.56 | O |
| ATOM | 2493 | N | PRO | A | 160 | 13.383 | 5.862 | 22.418 | 1.00 | 89.61 | N |
| ATOM | 2494 | CA | PRO | A | 160 | 14.743 | 6.031 | 22.954 | 1.00 | 91.86 | C |
| ATOM | 2496 | CB | PRO | A | 160 | 14.714 | 7.423 | 23.610 | 1.00 | 92.67 | C |
| ATOM | 2499 | CG | PRO | A | 160 | 13.501 | 8.102 | 23.127 | 1.00 | 90.33 | C |
| ATOM | 2502 | CD | PRO | A | 160 | 12.656 | 7.139 | 22.372 | 1.00 | 90.15 | C |
| ATOM | 2505 | C | PRO | A | 160 | 15.261 | 5.019 | 23.970 | 1.00 | 91.59 | C |
| ATOM | 2506 | O | PRO | A | 160 | 14.535 | 4.311 | 24.654 | 1.00 | 87.47 | O |
| ATOM | 2507 | N | LEU | A | 161 | 16.582 | 5.002 | 24.046 | 1.00 | 94.49 | N |
| ATOM | 2509 | CA | LEU | A | 161 | 17.318 | 4.103 | 24.906 | 1.00 | 97.03 | C |
| ATOM | 2511 | CB | LEU | A | 161 | 17.885 | 2.918 | 24.078 | 1.00 | 96.28 | C |
| ATOM | 2514 | CG | LEU | A | 161 | 17.017 | 1.707 | 23.671 | 1.00 | 94.82 | C |
| ATOM | 2516 | CD1 | LEU | A | 161 | 17.658 | 0.923 | 22.493 | 1.00 | 93.82 | C |
| ATOM | 2520 | CD2 | LEU | A | 161 | 16.735 | 0.783 | 24.864 | 1.00 | 93.08 | C |
| ATOM | 2524 | C | LEU | A | 161 | 18.475 | 4.906 | 25.503 | 1.00 | 98.82 | C |
| ATOM | 2525 | O | LEU | A | 161 | 18.848 | 5.967 | 24.987 | 1.00 | 98.25 | O |
| ATOM | 2526 | N | GLN | A | 162 | 19.013 | 4.404 | 26.608 | 1.00 | 101.00 | N |
| ATOM | 2528 | CA | GLN | A | 162 | 20.290 | 4.865 | 27.135 | 1.00 | 101.95 | C |
| ATOM | 2530 | CB | GLN | A | 162 | 20.225 | 5.088 | 28.660 | 1.00 | 103.13 | C |
| ATOM | 2533 | CG | GLN | A | 162 | 19.060 | 5.934 | 29.201 | 1.00 | 102.96 | C |
| ATOM | 2536 | CD | GLN | A | 162 | 18.651 | 5.522 | 30.624 | 1.00 | 104.66 | C |
| ATOM | 2537 | OE1 | GLN | A | 162 | 17.703 | 6.076 | 31.190 | 1.00 | 104.03 | O |
| ATOM | 2538 | NE2 | GLN | A | 162 | 19.365 | 4.548 | 31.196 | 1.00 | 103.68 | N |
| ATOM | 2541 | C | GLN | A | 162 | 21.315 | 3.748 | 26.850 | 1.00 | 102.26 | C |
| ATOM | 2542 | O | GLN | A | 162 | 20.958 | 2.612 | 26.522 | 1.00 | 99.57 | O |
| ATOM | 2543 | N | GLN | A | 163 | 22.593 | 4.079 | 26.965 | 1.00 | 104.53 | N |
| ATOM | 2545 | CA | GLN | A | 163 | 23.655 | 3.073 | 26.995 | 1.00 | 105.21 | C |
| ATOM | 2547 | CB | GLN | A | 163 | 24.906 | 3.716 | 27.567 | 1.00 | 105.72 | C |
| ATOM | 2550 | CG | GLN | A | 163 | 26.028 | 2.741 | 27.928 | 1.00 | 105.46 | C |
| ATOM | 2553 | CD | GLN | A | 163 | 27.384 | 3.435 | 27.930 | 1.00 | 103.07 | C |
| ATOM | 2554 | OE1 | GLN | A | 163 | 27.767 | 4.048 | 28.924 | 1.00 | 96.11 | O |
| ATOM | 2555 | NE2 | GLN | A | 163 | 28.105 | 3.345 | 26.814 | 1.00 | 101.46 | N |
| ATOM | 2558 | C | GLN | A | 163 | 23.314 | 1.874 | 27.894 | 1.00 | 105.84 | C |
| ATOM | 2559 | O | GLN | A | 163 | 23.625 | 0.716 | 27.561 | 1.00 | 105.65 | O |
| ATOM | 2560 | N | SER | A | 164 | 22.684 | 2.167 | 29.034 | 1.00 | 105.53 | N |
| ATOM | 2562 | CA | SER | A | 164 | 22.315 | 1.146 | 30.020 | 1.00 | 105.77 | C |
| ATOM | 2564 | CB | SER | A | 164 | 21.632 | 1.798 | 31.227 | 1.00 | 106.94 | C |
| ATOM | 2567 | OG | SER | A | 164 | 21.798 | 1.017 | 32.400 | 1.00 | 107.14 | O |
| ATOM | 2569 | C | SER | A | 164 | 21.335 | 0.192 | 29.374 | 1.00 | 105.64 | C |
| ATOM | 2570 | O | SER | A | 164 | 21.407 | −1.065 | 29.492 | 1.00 | 105.75 | O |
| ATOM | 2571 | N | GLU | A | 165 | 20.403 | 0.835 | 28.682 | 1.00 | 102.30 | N |
| ATOM | 2573 | CA | GLU | A | 165 | 19.381 | 0.105 | 27.976 | 1.00 | 98.16 | C |
| ATOM | 2575 | CB | GLU | A | 165 | 18.355 | 1.082 | 27.415 | 1.00 | 98.15 | C |
| ATOM | 2578 | CG | GLU | A | 165 | 17.997 | 2.126 | 28.479 | 1.00 | 99.91 | C |
| ATOM | 2581 | CD | GLU | A | 165 | 16.584 | 2.687 | 28.403 | 1.00 | 98.66 | C |
| ATOM | 2582 | OE1 | GLU | A | 165 | 15.621 | 1.933 | 28.642 | 1.00 | 94.69 | O |
| ATOM | 2583 | OE2 | GLU | A | 165 | 16.444 | 3.900 | 28.123 | 1.00 | 100.25 | O |
| ATOM | 2584 | C | GLU | A | 165 | 20.072 | −0.690 | 26.891 | 1.00 | 95.52 | C |
| ATOM | 2585 | O | GLU | A | 165 | 19.903 | −1.909 | 26.807 | 1.00 | 92.32 | O |
| ATOM | 2586 | N | VAL | A | 166 | 20.884 | 0.002 | 26.095 | 1.00 | 92.23 | N |
| ATOM | 2588 | CA | VAL | A | 166 | 21.552 | −0.618 | 24.959 | 1.00 | 90.08 | C |
| ATOM | 2590 | CB | VAL | A | 166 | 22.637 | 0.280 | 24.385 | 1.00 | 89.46 | C |
| ATOM | 2592 | CG1 | VAL | A | 166 | 23.997 | −0.471 | 24.238 | 1.00 | 89.54 | C |
| ATOM | 2596 | CG2 | VAL | A | 166 | 22.180 | 0.834 | 23.067 | 1.00 | 89.64 | C |
| ATOM | 2600 | C | VAL | A | 166 | 22.155 | −1.969 | 25.275 | 1.00 | 89.54 | C |
| ATOM | 2601 | O | VAL | A | 166 | 21.868 | −2.949 | 24.587 | 1.00 | 90.45 | O |
| ATOM | 2602 | N | LEU | A | 167 | 22.988 | −2.039 | 26.306 | 1.00 | 89.05 | N |
| ATOM | 2604 | CA | LEU | A | 167 | 23.644 | −3.308 | 26.620 | 1.00 | 88.10 | C |
| ATOM | 2606 | CB | LEU | A | 167 | 24.572 | −3.201 | 27.852 | 1.00 | 87.51 | C |
| ATOM | 2609 | CG | LEU | A | 167 | 25.665 | −2.130 | 27.780 | 1.00 | 89.06 | C |
| ATOM | 2611 | CD1 | LEU | A | 167 | 25.990 | −1.526 | 29.151 | 1.00 | 88.01 | C |
| ATOM | 2615 | CD2 | LEU | A | 167 | 26.921 | −2.670 | 27.122 | 1.00 | 88.50 | C |
| ATOM | 2619 | C | LEU | A | 167 | 22.606 | −4.403 | 26.851 | 1.00 | 84.50 | C |
| ATOM | 2620 | O | LEU | A | 167 | 22.770 | −5.521 | 26.386 | 1.00 | 82.46 | O |
| ATOM | 2621 | N | ALA | A | 168 | 21.533 | −4.071 | 27.554 | 1.00 | 82.79 | N |
| ATOM | 2623 | CA | ALA | A | 168 | 20.565 | −5.083 | 27.974 | 1.00 | 83.99 | C |
| ATOM | 2625 | CB | ALA | A | 168 | 19.807 | −4.567 | 29.200 | 1.00 | 84.06 | C |
| ATOM | 2629 | C | ALA | A | 168 | 19.560 | −5.604 | 26.929 | 1.00 | 84.00 | C |
| ATOM | 2630 | O | ALA | A | 168 | 18.918 | −6.627 | 27.179 | 1.00 | 83.07 | O |
| ATOM | 2631 | N | SER | A | 169 | 19.423 | −4.940 | 25.776 | 1.00 | 82.75 | N |
| ATOM | 2633 | CA | SER | A | 169 | 18.339 | −5.295 | 24.845 | 1.00 | 82.41 | C |
| ATOM | 2635 | CB | SER | A | 169 | 17.199 | −4.258 | 24.968 | 1.00 | 83.85 | C |
| ATOM | 2638 | OG | SER | A | 169 | 16.810 | −4.032 | 26.327 | 1.00 | 80.23 | O |
| ATOM | 2640 | C | SER | A | 169 | 18.735 | −5.521 | 23.349 | 1.00 | 81.77 | C |
| ATOM | 2641 | O | SER | A | 169 | 19.377 | −4.675 | 22.718 | 1.00 | 80.20 | O |
| ATOM | 2642 | N | VAL | A | 170 | 18.303 | −6.674 | 22.816 | 1.00 | 79.25 | N |
| ATOM | 2644 | CA | VAL | A | 170 | 18.634 | −7.170 | 21.469 | 1.00 | 74.78 | C |
| ATOM | 2646 | CB | VAL | A | 170 | 17.885 | −8.506 | 21.198 | 1.00 | 76.32 | C |
| ATOM | 2648 | CG1 | VAL | A | 170 | 18.278 | −9.111 | 19.831 | 1.00 | 75.02 | C |
| ATOM | 2652 | CG2 | VAL | A | 170 | 18.143 | −9.504 | 22.334 | 1.00 | 77.47 | C |
| ATOM | 2656 | C | VAL | A | 170 | 18.329 | −6.185 | 20.326 | 1.00 | 70.08 | C |
| ATOM | 2657 | O | VAL | A | 170 | 17.197 | −5.720 | 20.160 | 1.00 | 70.47 | O |
| ATOM | 2658 | N | GLY | A | 171 | 19.345 | −5.895 | 19.525 | 1.00 | 58.93 | N |
| ATOM | 2660 | CA | GLY | A | 171 | 19.208 | −4.921 | 18.482 | 1.00 | 51.34 | C |
| ATOM | 2663 | C | GLY | A | 171 | 19.305 | −5.525 | 17.115 | 1.00 | 47.82 | C |
| ATOM | 2664 | O | GLY | A | 171 | 19.166 | −4.812 | 16.149 | 1.00 | 51.16 | O |
| ATOM | 2665 | N | GLY | A | 172 | 19.548 | −6.821 | 16.992 | 1.00 | 43.16 | N |
| ATOM | 2667 | CA | GLY | A | 172 | 19.604 | −7.381 | 15.654 | 1.00 | 40.82 | C |
| ATOM | 2670 | C | GLY | A | 172 | 20.369 | −8.678 | 15.473 | 1.00 | 42.31 | C |
| ATOM | 2671 | O | GLY | A | 172 | 20.653 | −9.404 | 16.439 | 1.00 | 39.19 | O |
| ATOM | 2672 | N | SER | A | 173 | 20.705 | −8.960 | 14.214 | 1.00 | 39.65 | N |
| ATOM | 2674 | CA | SER | A | 173 | 21.301 | −10.233 | 13.857 | 1.00 | 37.97 | C |
| ATOM | 2676 | CB | SER | A | 173 | 20.194 | −11.203 | 13.497 | 1.00 | 36.33 | C |
| ATOM | 2679 | OG | SER | A | 173 | 19.416 | −11.435 | 14.634 | 1.00 | 46.54 | O |
| ATOM | 2681 | C | SER | A | 173 | 22.205 | −10.181 | 12.664 | 1.00 | 39.01 | C |
| ATOM | 2682 | O | SER | A | 173 | 21.805 | −9.673 | 11.590 | 1.00 | 36.11 | O |
| ATOM | 2683 | N | MET | A | 174 | 23.411 | −10.705 | 12.853 | 1.00 | 35.19 | N |
| ATOM | 2685 | CA | MET | A | 174 | 24.339 | −10.883 | 11.749 | 1.00 | 40.44 | C |
| ATOM | 2687 | CB | MET | A | 174 | 25.676 | −10.261 | 12.040 | 1.00 | 40.55 | C |
| ATOM | 2690 | CG | MET | A | 174 | 26.481 | −10.176 | 10.760 | 1.00 | 45.60 | C |
| ATOM | 2693 | SD | MET | A | 174 | 28.136 | −9.627 | 10.960 | 1.00 | 47.63 | S |
| ATOM | 2694 | CE | MET | A | 174 | 28.387 | −10.094 | 12.700 | 1.00 | 54.56 | C |
| ATOM | 2698 | C | MET | A | 174 | 24.483 | −12.394 | 11.516 | 1.00 | 41.84 | C |
| ATOM | 2699 | O | MET | A | 174 | 25.013 | −13.111 | 12.356 | 1.00 | 48.31 | O |
| ATOM | 2700 | N | ILE | A | 175 | 24.003 | −12.875 | 10.383 | 1.00 | 38.41 | N |
| ATOM | 2702 | CA | ILE | A | 175 | 23.939 | −14.288 | 10.136 | 1.00 | 37.39 | C |
| ATOM | 2704 | CB | ILE | A | 175 | 22.628 | −14.676 | 9.443 | 1.00 | 39.69 | C |
| ATOM | 2706 | CG1 | ILE | A | 175 | 21.448 | −14.387 | 10.364 | 1.00 | 40.97 | C |
| ATOM | 2709 | CD1 | ILE | A | 175 | 21.449 | −15.213 | 11.578 | 1.00 | 36.79 | C |
| ATOM | 2713 | CG2 | ILE | A | 175 | 22.653 | −16.089 | 9.035 | 1.00 | 35.35 | C |
| ATOM | 2717 | C | ILE | A | 175 | 25.021 | −14.522 | 9.202 | 1.00 | 38.73 | C |
| ATOM | 2718 | O | ILE | A | 175 | 24.892 | −14.246 | 8.037 | 1.00 | 40.30 | O |
| ATOM | 2719 | N | ILE | A | 176 | 26.109 | −15.038 | 9.722 | 1.00 | 42.09 | N |
| ATOM | 2721 | CA | ILE | A | 176 | 27.257 | −15.301 | 8.926 | 1.00 | 37.74 | C |
| ATOM | 2723 | CB | ILE | A | 176 | 28.382 | −15.377 | 9.844 | 1.00 | 35.36 | C |
| ATOM | 2725 | CG1 | ILE | A | 176 | 28.371 | −14.125 | 10.699 | 1.00 | 32.86 | C |
| ATOM | 2728 | CD1 | ILE | A | 176 | 29.740 | −13.602 | 11.051 | 1.00 | 37.77 | C |
| ATOM | 2732 | CG2 | ILE | A | 176 | 29.673 | −15.621 | 9.060 | 1.00 | 38.65 | C |
| ATOM | 2736 | C | ILE | A | 176 | 27.119 | −16.617 | 8.202 | 1.00 | 43.85 | C |
| ATOM | 2737 | O | ILE | A | 176 | 27.178 | −17.681 | 8.802 | 1.00 | 49.67 | O |
| ATOM | 2738 | N | GLY | A | 177 | 26.890 | −16.568 | 6.912 | 1.00 | 44.93 | N |
| ATOM | 2740 | CA | GLY | A | 177 | 27.003 | −17.783 | 6.139 | 1.00 | 45.46 | C |
| ATOM | 2743 | C | GLY | A | 177 | 25.734 | −18.428 | 5.673 | 1.00 | 42.70 | C |
| ATOM | 2744 | O | GLY | A | 177 | 25.751 | −19.498 | 5.054 | 1.00 | 47.24 | O |
| ATOM | 2745 | N | GLY | A | 178 | 24.619 | −17.791 | 5.934 | 1.00 | 40.94 | N |
| ATOM | 2747 | CA | GLY | A | 178 | 23.369 | −18.400 | 5.553 | 1.00 | 38.84 | C |
| ATOM | 2750 | C | GLY | A | 178 | 22.355 | −17.317 | 5.705 | 1.00 | 36.04 | C |
| ATOM | 2751 | O | GLY | A | 178 | 22.754 | −16.229 | 6.066 | 1.00 | 32.65 | O |
| ATOM | 2752 | N | ILE | A | 179 | 21.086 | −17.636 | 5.418 | 1.00 | 38.29 | N |
| ATOM | 2754 | CA | ILE | A | 179 | 19.922 | −16.748 | 5.502 | 1.00 | 36.73 | C |
| ATOM | 2756 | CB | ILE | A | 179 | 19.262 | −16.733 | 4.137 | 1.00 | 38.18 | C |
| ATOM | 2758 | CG1 | ILE | A | 179 | 20.269 | −16.324 | 3.069 | 1.00 | 38.79 | C |
| ATOM | 2761 | CD1 | ILE | A | 179 | 19.789 | −16.538 | 1.644 | 1.00 | 37.73 | C |
| ATOM | 2765 | CG2 | ILE | A | 179 | 18.144 | −15.825 | 4.105 | 1.00 | 40.58 | C |
| ATOM | 2769 | C | ILE | A | 179 | 19.019 | −17.446 | 6.489 | 1.00 | 39.59 | C |
| ATOM | 2770 | O | ILE | A | 179 | 18.914 | −18.666 | 6.450 | 1.00 | 42.86 | O |
| ATOM | 2771 | N | ASP | A | 180 | 18.355 | −16.727 | 7.387 | 1.00 | 43.92 | N |
| ATOM | 2773 | CA | ASP | A | 180 | 17.497 | −17.405 | 8.388 | 1.00 | 40.32 | C |
| ATOM | 2775 | CB | ASP | A | 180 | 17.913 | −17.030 | 9.788 | 1.00 | 42.54 | C |
| ATOM | 2778 | CG | ASP | A | 180 | 16.967 | −17.607 | 10.824 | 1.00 | 45.20 | C |
| ATOM | 2779 | OD1 | ASP | A | 180 | 17.452 | −17.999 | 11.899 | 1.00 | 38.88 | O |
| ATOM | 2780 | OD2 | ASP | A | 180 | 15.717 | −17.695 | 10.630 | 1.00 | 43.52 | O |
| ATOM | 2781 | C | ASP | A | 180 | 16.012 | −17.138 | 8.278 | 1.00 | 38.18 | C |
| ATOM | 2782 | O | ASP | A | 180 | 15.533 | −16.094 | 8.669 | 1.00 | 44.65 | O |
| ATOM | 2783 | N | HIS | A | 181 | 15.272 | −18.105 | 7.782 | 1.00 | 42.63 | N |
| ATOM | 2785 | CA | HIS | A | 181 | 13.864 | −17.928 | 7.490 | 1.00 | 41.71 | C |
| ATOM | 2787 | CB | HIS | A | 181 | 13.320 | −19.226 | 6.973 | 1.00 | 48.30 | C |
| ATOM | 2790 | CG | HIS | A | 181 | 13.911 | −19.569 | 5.657 | 1.00 | 63.31 | C |
| ATOM | 2791 | ND1 | HIS | A | 181 | 13.525 | −18.933 | 4.495 | 1.00 | 74.50 | N |
| ATOM | 2793 | CE1 | HIS | A | 181 | 14.230 | −19.409 | 3.483 | 1.00 | 79.36 | C |
| ATOM | 2795 | NE2 | HIS | A | 181 | 15.065 | −20.328 | 3.950 | 1.00 | 79.27 | N |
| ATOM | 2797 | CD2 | HIS | A | 181 | 14.890 | −20.439 | 5.310 | 1.00 | 71.55 | C |
| ATOM | 2799 | C | HIS | A | 181 | 12.983 | −17.340 | 8.538 | 1.00 | 37.16 | C |
| ATOM | 2800 | O | HIS | A | 181 | 11.899 | −16.873 | 8.232 | 1.00 | 39.58 | O |
| ATOM | 2801 | N | SER | A | 182 | 13.421 | −17.329 | 9.773 | 1.00 | 37.68 | N |
| ATOM | 2803 | CA | SER | A | 182 | 12.603 | −16.703 | 10.793 | 1.00 | 41.51 | C |
| ATOM | 2805 | CB | SER | A | 182 | 12.986 | −17.234 | 12.149 | 1.00 | 42.17 | C |
| ATOM | 2808 | OG | SER | A | 182 | 14.344 | −16.913 | 12.339 | 1.00 | 43.89 | O |
| ATOM | 2810 | C | SER | A | 182 | 12.765 | −15.204 | 10.848 | 1.00 | 39.23 | C |
| ATOM | 2811 | O | SER | A | 182 | 12.013 | −14.548 | 11.554 | 1.00 | 46.71 | O |
| ATOM | 2812 | N | LEU | A | 183 | 13.730 | −14.644 | 10.130 | 1.00 | 38.97 | N |
| ATOM | 2814 | CA | LEU | A | 183 | 13.967 | −13.201 | 10.197 | 1.00 | 35.60 | C |
| ATOM | 2816 | CB | LEU | A | 183 | 15.456 | −12.924 | 10.189 | 1.00 | 33.38 | C |
| ATOM | 2819 | CG | LEU | A | 183 | 16.258 | −13.474 | 11.374 | 1.00 | 31.83 | C |
| ATOM | 2821 | CD1 | LEU | A | 183 | 17.709 | −13.477 | 11.088 | 1.00 | 31.76 | C |
| ATOM | 2825 | CD2 | LEU | A | 183 | 16.069 | −12.666 | 12.607 | 1.00 | 41.82 | C |
| ATOM | 2829 | C | LEU | A | 183 | 13.318 | −12.400 | 9.068 | 1.00 | 38.07 | C |
| ATOM | 2830 | O | LEU | A | 183 | 13.701 | −11.288 | 8.820 | 1.00 | 42.04 | O |
| ATOM | 2831 | N | TYR | A | 184 | 12.336 | −12.956 | 8.367 | 1.00 | 44.43 | N |
| ATOM | 2833 | CA | TYR | A | 184 | 11.639 | −12.209 | 7.320 | 1.00 | 33.34 | C |
| ATOM | 2835 | CB | TYR | A | 184 | 12.516 | −12.087 | 6.063 | 1.00 | 38.72 | C |
| ATOM | 2838 | CG | TYR | A | 184 | 12.775 | −13.281 | 5.134 | 1.00 | 35.86 | C |
| ATOM | 2839 | CD1 | TYR | A | 184 | 13.928 | −14.047 | 5.248 | 1.00 | 43.79 | C |
| ATOM | 2841 | CE1 | TYR | A | 184 | 14.184 | −15.099 | 4.406 | 1.00 | 39.71 | C |
| ATOM | 2843 | CZ | TYR | A | 184 | 13.294 | −15.400 | 3.426 | 1.00 | 40.90 | C |
| ATOM | 2844 | OH | TYR | A | 184 | 13.537 | −16.460 | 2.577 | 1.00 | 45.55 | O |
| ATOM | 2846 | CE2 | TYR | A | 184 | 12.159 | −14.656 | 3.277 | 1.00 | 38.72 | C |
| ATOM | 2848 | CD2 | TYR | A | 184 | 11.911 | −13.599 | 4.127 | 1.00 | 36.47 | C |
| ATOM | 2850 | C | TYR | A | 184 | 10.304 | −12.811 | 7.005 | 1.00 | 36.16 | C |
| ATOM | 2851 | O | TYR | A | 184 | 10.067 | −13.961 | 7.352 | 1.00 | 30.88 | O |
| ATOM | 2852 | N | THR | A | 185 | 9.419 | −12.023 | 6.383 | 1.00 | 37.93 | N |
| ATOM | 2854 | CA | THR | A | 185 | 8.171 | −12.549 | 5.846 | 1.00 | 40.81 | C |
| ATOM | 2856 | CB | THR | A | 185 | 6.922 | −11.913 | 6.441 | 1.00 | 43.15 | C |
| ATOM | 2858 | OG1 | THR | A | 185 | 6.918 | −10.491 | 6.207 | 1.00 | 43.97 | O |
| ATOM | 2860 | CG2 | THR | A | 185 | 6.843 | −12.140 | 7.970 | 1.00 | 42.48 | C |
| ATOM | 2864 | C | THR | A | 185 | 8.152 | −12.271 | 4.378 | 1.00 | 37.63 | C |
| ATOM | 2865 | O | THR | A | 185 | 8.862 | −11.408 | 3.929 | 1.00 | 36.37 | O |
| ATOM | 2866 | N | GLY | A | 186 | 7.321 | −12.992 | 3.637 | 1.00 | 34.86 | N |
| ATOM | 2868 | CA | GLY | A | 186 | 7.221 | −12.791 | 2.205 | 1.00 | 37.11 | C |
| ATOM | 2871 | C | GLY | A | 186 | 8.449 | −13.300 | 1.469 | 1.00 | 38.69 | C |
| ATOM | 2872 | O | GLY | A | 186 | 9.179 | −14.161 | 1.954 | 1.00 | 44.04 | O |
| ATOM | 2873 | N | SER | A | 187 | 8.667 | −12.736 | 0.288 | 1.00 | 41.42 | N |
| ATOM | 2875 | CA | SER | A | 187 | 9.797 | −13.062 | −0.552 | 1.00 | 43.18 | C |
| ATOM | 2877 | CB | SER | A | 187 | 9.346 | −13.063 | −1.989 | 1.00 | 50.00 | C |
| ATOM | 2880 | OG | SER | A | 187 | 8.254 | −13.936 | −2.139 | 1.00 | 54.96 | O |
| ATOM | 2882 | C | SER | A | 187 | 10.934 | −12.059 | −0.443 | 1.00 | 44.84 | C |
| ATOM | 2883 | O | SER | A | 187 | 10.749 | −10.938 | 0.064 | 1.00 | 42.30 | O |
| ATOM | 2884 | N | LEU | A | 188 | 12.094 | −12.514 | −0.943 | 1.00 | 39.28 | N |
| ATOM | 2886 | CA | LEU | A | 188 | 13.369 | −11.810 | −1.031 | 1.00 | 33.66 | C |
| ATOM | 2888 | CB | LEU | A | 188 | 14.482 | −12.824 | −0.789 | 1.00 | 28.67 | C |
| ATOM | 2891 | CG | LEU | A | 188 | 15.028 | −12.884 | 0.614 | 1.00 | 36.42 | C |
| ATOM | 2893 | CD1 | LEU | A | 188 | 15.736 | −14.211 | 0.813 | 1.00 | 40.88 | C |
| ATOM | 2897 | CD2 | LEU | A | 188 | 15.971 | −11.777 | 0.889 | 1.00 | 34.49 | C |
| ATOM | 2901 | C | LEU | A | 188 | 13.515 | −11.417 | −2.474 | 1.00 | 31.52 | C |
| ATOM | 2902 | O | LEU | A | 188 | 13.359 | −12.268 | −3.294 | 1.00 | 41.60 | O |
| ATOM | 2903 | N | TRP | A | 189 | 13.803 | −10.177 | −2.822 | 1.00 | 31.35 | N |
| ATOM | 2905 | CA | TRP | A | 189 | 14.040 | −9.830 | −4.230 | 1.00 | 33.70 | C |
| ATOM | 2907 | CB | TRP | A | 189 | 13.132 | −8.688 | −4.683 | 1.00 | 39.33 | C |
| ATOM | 2910 | CG | TRP | A | 189 | 11.729 | −9.074 | −4.947 | 1.00 | 37.95 | C |
| ATOM | 2911 | CD1 | TRP | A | 189 | 10.779 | −9.313 | −4.033 | 1.00 | 35.87 | C |
| ATOM | 2913 | NE1 | TRP | A | 189 | 9.600 | −9.646 | −4.649 | 1.00 | 34.16 | N |
| ATOM | 2915 | CE2 | TRP | A | 189 | 9.790 | −9.626 | −6.001 | 1.00 | 38.81 | C |
| ATOM | 2916 | CD2 | TRP | A | 189 | 11.125 | −9.267 | −6.224 | 1.00 | 35.31 | C |
| ATOM | 2917 | CE3 | TRP | A | 189 | 11.586 | −9.181 | −7.539 | 1.00 | 35.10 | C |
| ATOM | 2919 | CZ3 | TRP | A | 189 | 10.725 | −9.447 | −8.559 | 1.00 | 43.54 | C |
| ATOM | 2921 | CH2 | TRP | A | 189 | 9.384 | −9.805 | −8.305 | 1.00 | 44.03 | C |
| ATOM | 2923 | CZ2 | TRP | A | 189 | 8.904 | −9.898 | −7.034 | 1.00 | 40.92 | C |
| ATOM | 2925 | C | TRP | A | 189 | 15.493 | −9.361 | −4.344 | 1.00 | 34.18 | C |
| ATOM | 2926 | O | TRP | A | 189 | 15.950 | −8.570 | −3.567 | 1.00 | 34.73 | O |
| ATOM | 2927 | N | TYR | A | 190 | 16.217 | −9.852 | −5.317 | 1.00 | 34.76 | N |
| ATOM | 2929 | CA | TYR | A | 190 | 17.603 | −9.539 | −5.412 | 1.00 | 36.58 | C |
| ATOM | 2931 | CB | TYR | A | 190 | 18.341 | −10.800 | −5.769 | 1.00 | 38.76 | C |
| ATOM | 2934 | CG | TYR | A | 190 | 18.382 | −11.826 | −4.657 | 1.00 | 40.52 | C |
| ATOM | 2935 | CD1 | TYR | A | 190 | 17.485 | −12.875 | −4.609 | 1.00 | 43.16 | C |
| ATOM | 2937 | CE1 | TYR | A | 190 | 17.541 | −13.817 | −3.580 | 1.00 | 43.91 | C |
| ATOM | 2939 | CZ | TYR | A | 190 | 18.506 | −13.685 | −2.615 | 1.00 | 42.77 | C |
| ATOM | 2940 | OH | TYR | A | 190 | 18.608 | −14.594 | −1.566 | 1.00 | 46.77 | O |
| ATOM | 2942 | CE2 | TYR | A | 190 | 19.384 | −12.642 | −2.677 | 1.00 | 35.63 | C |
| ATOM | 2944 | CD2 | TYR | A | 190 | 19.327 | −11.742 | −3.668 | 1.00 | 35.67 | C |
| ATOM | 2946 | C | TYR | A | 190 | 17.886 | −8.451 | −6.430 | 1.00 | 38.30 | C |
| ATOM | 2947 | O | TYR | A | 190 | 17.248 | −8.420 | −7.424 | 1.00 | 34.41 | O |
| ATOM | 2948 | N | THR | A | 191 | 18.850 | −7.567 | −6.133 | 1.00 | 41.18 | N |
| ATOM | 2950 | CA | THR | A | 191 | 19.299 | −6.512 | −7.025 | 1.00 | 37.26 | C |
| ATOM | 2952 | CB | THR | A | 191 | 19.056 | −5.091 | −6.438 | 1.00 | 38.25 | C |
| ATOM | 2954 | OG1 | THR | A | 191 | 19.283 | −4.111 | −7.448 | 1.00 | 41.41 | O |
| ATOM | 2956 | CG2 | THR | A | 191 | 20.057 | −4.689 | −5.419 | 1.00 | 31.07 | C |
| ATOM | 2960 | C | THR | A | 191 | 20.782 | −6.721 | −7.200 | 1.00 | 39.94 | C |
| ATOM | 2961 | O | THR | A | 191 | 21.489 | −7.008 | −6.264 | 1.00 | 41.73 | O |
| ATOM | 2962 | N | PRO | A | 192 | 21.281 | −6.578 | −8.395 | 1.00 | 39.23 | N |
| ATOM | 2963 | CA | PRO | A | 192 | 22.679 | −6.840 | −8.617 | 1.00 | 39.19 | C |
| ATOM | 2965 | CB | PRO | A | 192 | 22.824 | −6.565 | −10.111 | 1.00 | 41.47 | C |
| ATOM | 2968 | CG | PRO | A | 192 | 21.484 | −6.730 | −10.671 | 1.00 | 36.70 | C |
| ATOM | 2971 | CD | PRO | A | 192 | 20.603 | −6.152 | −9.629 | 1.00 | 41.63 | C |
| ATOM | 2974 | C | PRO | A | 192 | 23.568 | −5.885 | −7.860 | 1.00 | 41.91 | C |
| ATOM | 2975 | O | PRO | A | 192 | 23.164 | −4.808 | −7.531 | 1.00 | 43.47 | O |
| ATOM | 2976 | N | ILE | A | 193 | 24.784 | −6.324 | −7.580 | 1.00 | 46.52 | N |
| ATOM | 2978 | CA | ILE | A | 193 | 25.827 | −5.467 | −7.076 | 1.00 | 42.19 | C |
| ATOM | 2980 | CB | ILE | A | 193 | 26.764 | −6.250 | −6.239 | 1.00 | 38.18 | C |
| ATOM | 2982 | CG1 | ILE | A | 193 | 26.159 | −6.585 | −4.906 | 1.00 | 41.41 | C |
| ATOM | 2985 | CD1 | ILE | A | 193 | 27.036 | −7.645 | −4.107 | 1.00 | 45.77 | C |
| ATOM | 2989 | CG2 | ILE | A | 193 | 27.961 | −5.447 | −5.994 | 1.00 | 42.22 | C |
| ATOM | 2993 | C | ILE | A | 193 | 26.603 | −5.049 | −8.313 | 1.00 | 44.50 | C |
| ATOM | 2994 | O | ILE | A | 193 | 27.213 | −5.903 | −8.984 | 1.00 | 44.15 | O |
| ATOM | 2995 | N | ARG | A | 194 | 26.596 | −3.762 | −8.626 | 1.00 | 44.93 | N |
| ATOM | 2997 | CA | ARG | A | 194 | 27.234 | −3.288 | −9.846 | 1.00 | 47.47 | C |
| ATOM | 2999 | CB | ARG | A | 194 | 26.904 | −1.813 | −10.032 | 1.00 | 52.04 | C |
| ATOM | 3002 | CG | ARG | A | 194 | 27.273 | −1.190 | −11.424 | 1.00 | 54.84 | C |
| ATOM | 3005 | CD | ARG | A | 194 | 27.414 | 0.291 | −11.320 | 1.00 | 51.97 | C |
| ATOM | 3008 | NE | ARG | A | 194 | 27.329 | 1.055 | −12.548 | 1.00 | 49.01 | N |
| ATOM | 3010 | CZ | ARG | A | 194 | 27.591 | 2.367 | −12.600 | 1.00 | 45.93 | C |
| ATOM | 3011 | NH1 | ARG | A | 194 | 27.960 | 3.039 | −11.509 | 1.00 | 41.67 | N |
| ATOM | 3014 | NH2 | ARG | A | 194 | 27.492 | 3.018 | −13.742 | 1.00 | 51.03 | N |
| ATOM | 3017 | C | ARG | A | 194 | 28.763 | −3.469 | −9.869 | 1.00 | 53.38 | C |
| ATOM | 3018 | O | ARG | A | 194 | 29.331 | −3.747 | −10.924 | 1.00 | 57.83 | O |
| ATOM | 3019 | N | ARG | A | 195 | 29.433 | −3.341 | −8.723 | 1.00 | 49.23 | N |
| ATOM | 3021 | CA | ARG | A | 195 | 30.887 | −3.378 | −8.698 | 1.00 | 49.56 | C |
| ATOM | 3023 | CB | ARG | A | 195 | 31.470 | −1.973 | −8.965 | 1.00 | 49.15 | C |
| ATOM | 3026 | CG | ARG | A | 195 | 32.684 | −1.929 | −9.832 | 1.00 | 45.49 | C |
| ATOM | 3029 | CD | ARG | A | 195 | 33.888 | −1.211 | −9.229 | 1.00 | 54.35 | C |
| ATOM | 3032 | NE | ARG | A | 195 | 33.658 | 0.142 | −8.774 | 1.00 | 52.34 | N |
| ATOM | 3034 | CZ | ARG | A | 195 | 34.625 | 0.982 | −8.430 | 1.00 | 58.59 | C |
| ATOM | 3035 | NH1 | ARG | A | 195 | 35.892 | 0.624 | −8.476 | 1.00 | 59.01 | N |
| ATOM | 3038 | NH2 | ARG | A | 195 | 34.335 | 2.210 | −8.029 | 1.00 | 62.23 | N |
| ATOM | 3041 | C | ARG | A | 195 | 31.317 | −3.740 | −7.314 | 1.00 | 51.05 | C |
| ATOM | 3042 | O | ARG | A | 195 | 30.795 | −3.169 | −6.362 | 1.00 | 44.37 | O |
| ATOM | 3043 | N | GLU | A | 196 | 32.281 | −4.648 | −7.173 | 1.00 | 52.25 | N |
| ATOM | 3045 | CA | GLU | A | 196 | 32.723 | −4.988 | −5.831 | 1.00 | 53.43 | C |
| ATOM | 3047 | CB | GLU | A | 196 | 33.192 | −6.461 | −5.735 | 1.00 | 57.30 | C |
| ATOM | 3050 | CG | GLU | A | 196 | 32.048 | −7.476 | −5.923 | 1.00 | 59.93 | C |
| ATOM | 3053 | CD | GLU | A | 196 | 32.485 | −8.961 | −5.919 | 1.00 | 65.83 | C |
| ATOM | 3054 | OE1 | GLU | A | 196 | 32.977 | −9.443 | −6.966 | 1.00 | 59.79 | O |
| ATOM | 3055 | OE2 | GLU | A | 196 | 32.327 | −9.670 | −4.885 | 1.00 | 64.02 | O |
| ATOM | 3056 | C | GLU | A | 196 | 33.758 | −3.977 | −5.341 | 1.00 | 47.93 | C |
| ATOM | 3057 | O | GLU | A | 196 | 34.933 | −4.143 | −5.563 | 1.00 | 45.07 | O |
| ATOM | 3058 | N | TRP | A | 197 | 33.274 | −2.911 | −4.694 | 1.00 | 44.26 | N |
| ATOM | 3060 | CA | TRP | A | 197 | 34.115 | −1.929 | −4.021 | 1.00 | 39.52 | C |
| ATOM | 3062 | CB | TRP | A | 197 | 34.594 | −0.730 | −4.922 | 1.00 | 45.44 | C |
| ATOM | 3065 | CG | TRP | A | 197 | 33.660 | 0.246 | −5.517 | 1.00 | 40.87 | C |
| ATOM | 3066 | CD1 | TRP | A | 197 | 32.438 | 0.002 | −5.994 | 1.00 | 49.82 | C |
| ATOM | 3068 | NE1 | TRP | A | 197 | 31.860 | 1.157 | −6.468 | 1.00 | 45.72 | N |
| ATOM | 3070 | CE2 | TRP | A | 197 | 32.731 | 2.186 | −6.302 | 1.00 | 42.13 | C |
| ATOM | 3071 | CD2 | TRP | A | 197 | 33.885 | 1.649 | −5.705 | 1.00 | 45.88 | C |
| ATOM | 3072 | CE3 | TRP | A | 197 | 34.945 | 2.509 | −5.427 | 1.00 | 46.01 | C |
| ATOM | 3074 | CZ3 | TRP | A | 197 | 34.813 | 3.842 | −5.746 | 1.00 | 47.92 | C |
| ATOM | 3076 | CH2 | TRP | A | 197 | 33.651 | 4.330 | −6.338 | 1.00 | 50.66 | C |
| ATOM | 3078 | CZ2 | TRP | A | 197 | 32.600 | 3.515 | −6.626 | 1.00 | 43.28 | C |
| ATOM | 3080 | C | TRP | A | 197 | 33.334 | −1.576 | −2.764 | 1.00 | 38.50 | C |
| ATOM | 3081 | O | TRP | A | 197 | 33.528 | −2.184 | −1.733 | 1.00 | 49.55 | O |
| ATOM | 3082 | N | TYR | A | 198 | 32.472 | −0.599 | −2.786 | 1.00 | 38.10 | N |
| ATOM | 3084 | CA | TYR | A | 198 | 31.466 | −0.531 | −1.734 | 1.00 | 37.22 | C |
| ATOM | 3086 | CB | TYR | A | 198 | 30.770 | 0.851 | −1.643 | 1.00 | 35.77 | C |
| ATOM | 3089 | CG | TYR | A | 198 | 31.675 | 2.028 | −1.405 | 1.00 | 36.02 | C |
| ATOM | 3090 | CD1 | TYR | A | 198 | 31.813 | 2.562 | −0.142 | 1.00 | 38.94 | C |
| ATOM | 3092 | CE1 | TYR | A | 198 | 32.633 | 3.626 | 0.094 | 1.00 | 39.66 | C |
| ATOM | 3094 | CZ | TYR | A | 198 | 33.343 | 4.193 | −0.948 | 1.00 | 44.31 | C |
| ATOM | 3095 | OH | TYR | A | 198 | 34.174 | 5.269 | −0.701 | 1.00 | 46.07 | O |
| ATOM | 3097 | CE2 | TYR | A | 198 | 33.225 | 3.681 | −2.233 | 1.00 | 44.86 | C |
| ATOM | 3099 | CD2 | TYR | A | 198 | 32.386 | 2.595 | −2.448 | 1.00 | 42.57 | C |
| ATOM | 3101 | C | TYR | A | 198 | 30.433 | −1.521 | −2.214 | 1.00 | 33.99 | C |
| ATOM | 3102 | O | TYR | A | 198 | 30.574 | −2.022 | −3.287 | 1.00 | 30.84 | O |
| ATOM | 3103 | N | TYR | A | 199 | 29.405 | −1.802 | −1.418 | 1.00 | 38.45 | N |
| ATOM | 3105 | CA | TYR | A | 199 | 28.256 | −2.557 | −1.900 | 1.00 | 37.49 | C |
| ATOM | 3107 | CB | TYR | A | 199 | 27.490 | −3.208 | −0.728 | 1.00 | 36.73 | C |
| ATOM | 3110 | CG | TYR | A | 199 | 28.233 | −4.329 | −0.047 | 1.00 | 32.48 | C |
| ATOM | 3111 | CD1 | TYR | A | 199 | 28.787 | −4.165 | 1.205 | 1.00 | 33.92 | C |
| ATOM | 3113 | CE1 | TYR | A | 199 | 29.453 | −5.141 | 1.822 | 1.00 | 32.06 | C |
| ATOM | 3115 | CZ | TYR | A | 199 | 29.573 | −6.299 | 1.193 | 1.00 | 32.26 | C |
| ATOM | 3116 | OH | TYR | A | 199 | 30.243 | −7.369 | 1.769 | 1.00 | 34.61 | O |
| ATOM | 3118 | CE2 | TYR | A | 199 | 29.024 | −6.485 | −0.047 | 1.00 | 33.09 | C |
| ATOM | 3120 | CD2 | TYR | A | 199 | 28.372 | −5.519 | −0.644 | 1.00 | 33.21 | C |
| ATOM | 3122 | C | TYR | A | 199 | 27.368 | −1.525 | −2.645 | 1.00 | 42.41 | C |
| ATOM | 3123 | O | TYR | A | 199 | 26.418 | −0.978 | −2.075 | 1.00 | 45.22 | O |
| ATOM | 3124 | N | GLU | A | 200 | 27.702 | −1.272 | −3.910 | 1.00 | 43.18 | N |
| ATOM | 3126 | CA | GLU | A | 200 | 27.054 | −0.284 | −4.777 | 1.00 | 43.16 | C |
| ATOM | 3128 | CB | GLU | A | 200 | 28.089 | 0.193 | −5.809 | 1.00 | 46.83 | C |
| ATOM | 3131 | CG | GLU | A | 200 | 27.626 | 1.383 | −6.660 | 1.00 | 52.95 | C |
| ATOM | 3134 | CD | GLU | A | 200 | 28.337 | 1.543 | −7.977 | 1.00 | 51.92 | C |
| ATOM | 3135 | OE1 | GLU | A | 200 | 29.496 | 1.164 | −8.085 | 1.00 | 56.62 | O |
| ATOM | 3136 | OE2 | GLU | A | 200 | 27.715 | 2.055 | −8.901 | 1.00 | 54.72 | O |
| ATOM | 3137 | C | GLU | A | 200 | 25.857 | −0.816 | −5.577 | 1.00 | 44.00 | C |
| ATOM | 3138 | O | GLU | A | 200 | 25.993 | −1.789 | −6.319 | 1.00 | 48.61 | O |
| ATOM | 3139 | N | VAL | A | 201 | 24.691 | −0.193 | −5.481 | 1.00 | 37.72 | N |
| ATOM | 3141 | CA | VAL | A | 201 | 23.583 | −0.713 | −6.250 | 1.00 | 38.06 | C |
| ATOM | 3143 | CB | VAL | A | 201 | 22.598 | −1.332 | −5.374 | 1.00 | 33.81 | C |
| ATOM | 3145 | CG1 | VAL | A | 201 | 23.127 | −2.629 | −4.926 | 1.00 | 36.02 | C |
| ATOM | 3149 | CG2 | VAL | A | 201 | 22.411 | −0.506 | −4.261 | 1.00 | 33.46 | C |
| ATOM | 3153 | C | VAL | A | 201 | 22.911 | 0.371 | −7.006 | 1.00 | 36.84 | C |
| ATOM | 3154 | O | VAL | A | 201 | 23.235 | 1.474 | −6.781 | 1.00 | 46.66 | O |
| ATOM | 3155 | N | ILE | A | 202 | 21.975 | 0.073 | −7.877 | 1.00 | 41.86 | N |
| ATOM | 3157 | CA | ILE | A | 202 | 21.271 | 1.122 | −8.619 | 1.00 | 45.62 | C |
| ATOM | 3159 | CB | ILE | A | 202 | 21.435 | 0.898 | −9.997 | 1.00 | 47.37 | C |
| ATOM | 3161 | CG1 | ILE | A | 202 | 22.924 | 0.945 | −10.223 | 1.00 | 50.21 | C |
| ATOM | 3164 | CD1 | ILE | A | 202 | 23.240 | 1.610 | −11.515 | 1.00 | 50.75 | C |
| ATOM | 3168 | CG2 | ILE | A | 202 | 20.619 | 2.021 | −10.700 | 1.00 | 47.14 | C |
| ATOM | 3172 | C | ILE | A | 202 | 19.779 | 1.335 | −8.517 | 1.00 | 46.10 | C |
| ATOM | 3173 | O | ILE | A | 202 | 19.001 | 0.401 | −8.788 | 1.00 | 46.34 | O |
| ATOM | 3174 | N | ILE | A | 203 | 19.438 | 2.595 | −8.195 | 1.00 | 44.21 | N |
| ATOM | 3176 | CA | ILE | A | 203 | 18.092 | 3.089 | −7.882 | 1.00 | 41.01 | C |
| ATOM | 3178 | CB | ILE | A | 203 | 18.195 | 4.290 | −6.931 | 1.00 | 42.75 | C |
| ATOM | 3180 | CG1 | ILE | A | 203 | 18.885 | 3.856 | −5.629 | 1.00 | 36.25 | C |
| ATOM | 3183 | CD1 | ILE | A | 203 | 18.487 | 4.666 | −4.472 | 1.00 | 37.62 | C |
| ATOM | 3187 | CG2 | ILE | A | 203 | 16.754 | 4.972 | −6.754 | 1.00 | 42.00 | C |
| ATOM | 3191 | C | ILE | A | 203 | 17.508 | 3.646 | −9.096 | 1.00 | 43.71 | C |
| ATOM | 3192 | O | ILE | A | 203 | 18.164 | 4.384 | −9.797 | 1.00 | 45.90 | O |
| ATOM | 3193 | N | VAL | A | 204 | 16.259 | 3.369 | −9.374 | 1.00 | 43.96 | N |
| ATOM | 3195 | CA | VAL | A | 204 | 15.807 | 3.811 | −10.662 | 1.00 | 43.46 | C |
| ATOM | 3197 | CB | VAL | A | 204 | 15.743 | 2.596 | −11.563 | 1.00 | 47.55 | C |
| ATOM | 3199 | CG1 | VAL | A | 204 | 17.031 | 1.774 | −11.395 | 1.00 | 51.23 | C |
| ATOM | 3203 | CG2 | VAL | A | 204 | 14.676 | 1.721 | −11.133 | 1.00 | 52.39 | C |
| ATOM | 3207 | C | VAL | A | 204 | 14.525 | 4.610 | −10.624 | 1.00 | 42.03 | C |
| ATOM | 3208 | O | VAL | A | 204 | 14.075 | 5.093 | −11.639 | 1.00 | 46.48 | O |
| ATOM | 3209 | N | ARG | A | 205 | 13.944 | 4.739 | −9.445 | 1.00 | 40.50 | N |
| ATOM | 3211 | CA | ARG | A | 205 | 12.755 | 5.532 | −9.254 | 1.00 | 41.19 | C |
| ATOM | 3213 | CB | ARG | A | 205 | 11.567 | 4.685 | −9.609 | 1.00 | 44.75 | C |
| ATOM | 3216 | CG | ARG | A | 205 | 10.235 | 5.385 | −9.532 | 1.00 | 49.25 | C |
| ATOM | 3219 | CD | ARG | A | 205 | 9.191 | 4.670 | −10.422 | 1.00 | 55.42 | C |
| ATOM | 3222 | NE | ARG | A | 205 | 7.775 | 4.754 | −10.007 | 1.00 | 57.55 | N |
| ATOM | 3224 | CZ | ARG | A | 205 | 6.805 | 5.190 | −10.797 | 1.00 | 56.49 | C |
| ATOM | 3225 | NH1 | ARG | A | 205 | 7.092 | 5.604 | −12.017 | 1.00 | 54.80 | N |
| ATOM | 3228 | NH2 | ARG | A | 205 | 5.550 | 5.215 | −10.377 | 1.00 | 61.53 | N |
| ATOM | 3231 | C | ARG | A | 205 | 12.674 | 5.953 | −7.799 | 1.00 | 38.57 | C |
| ATOM | 3232 | O | ARG | A | 205 | 13.264 | 5.307 | −7.003 | 1.00 | 40.72 | O |
| ATOM | 3233 | N | VAL | A | 206 | 11.961 | 7.032 | −7.462 | 1.00 | 39.00 | N |
| ATOM | 3235 | CA | VAL | A | 206 | 11.709 | 7.408 | −6.072 | 1.00 | 35.25 | C |
| ATOM | 3237 | CB | VAL | A | 206 | 12.689 | 8.391 | −5.629 | 1.00 | 38.80 | C |
| ATOM | 3239 | CG1 | VAL | A | 206 | 12.331 | 8.891 | −4.228 | 1.00 | 40.25 | C |
| ATOM | 3243 | CG2 | VAL | A | 206 | 14.073 | 7.800 | −5.636 | 1.00 | 45.84 | C |
| ATOM | 3247 | C | VAL | A | 206 | 10.285 | 8.022 | −5.864 | 1.00 | 41.79 | C |
| ATOM | 3248 | O | VAL | A | 206 | 9.881 | 9.004 | −6.540 | 1.00 | 41.15 | O |
| ATOM | 3249 | N | GLU | A | 207 | 9.521 | 7.462 | −4.919 | 1.00 | 43.20 | N |
| ATOM | 3251 | CA | GLU | A | 207 | 8.124 | 7.883 | −4.684 | 1.00 | 39.16 | C |
| ATOM | 3253 | CB | GLU | A | 207 | 7.169 | 6.726 | −4.872 | 1.00 | 41.81 | C |
| ATOM | 3256 | CG | GLU | A | 207 | 6.651 | 6.501 | −6.293 | 1.00 | 42.92 | C |
| ATOM | 3259 | CD | GLU | A | 207 | 5.854 | 5.214 | −6.417 | 1.00 | 41.92 | C |
| ATOM | 3260 | OE1 | GLU | A | 207 | 5.320 | 4.691 | −5.383 | 1.00 | 41.67 | O |
| ATOM | 3261 | OE2 | GLU | A | 207 | 5.772 | 4.738 | −7.560 | 1.00 | 41.81 | O |
| ATOM | 3262 | C | GLU | A | 207 | 7.891 | 8.349 | −3.299 | 1.00 | 39.38 | C |
| ATOM | 3263 | O | GLU | A | 207 | 8.440 | 7.804 | −2.362 | 1.00 | 40.98 | O |
| ATOM | 3264 | N | ILE | A | 208 | 7.056 | 9.361 | −3.150 | 1.00 | 42.71 | N |
| ATOM | 3266 | CA | ILE | A | 208 | 6.682 | 9.842 | −1.819 | 1.00 | 40.22 | C |
| ATOM | 3268 | CB | ILE | A | 208 | 7.109 | 11.296 | −1.692 | 1.00 | 40.78 | C |
| ATOM | 3270 | CG1 | ILE | A | 208 | 8.634 | 11.379 | −1.893 | 1.00 | 42.36 | C |
| ATOM | 3273 | CD1 | ILE | A | 208 | 9.464 | 11.215 | −0.613 | 1.00 | 45.04 | C |
| ATOM | 3277 | CG2 | ILE | A | 208 | 6.718 | 11.934 | −0.350 | 1.00 | 42.70 | C |
| ATOM | 3281 | C | ILE | A | 208 | 5.189 | 9.584 | −1.877 | 1.00 | 41.38 | C |
| ATOM | 3282 | O | ILE | A | 208 | 4.511 | 10.062 | −2.777 | 1.00 | 48.60 | O |
| ATOM | 3283 | N | ASN | A | 209 | 4.672 | 8.792 | −0.955 | 1.00 | 43.14 | N |
| ATOM | 3285 | CA | ASN | A | 209 | 3.297 | 8.295 | −1.071 | 1.00 | 45.99 | C |
| ATOM | 3287 | CB | ASN | A | 209 | 2.273 | 9.238 | −0.482 | 1.00 | 45.44 | C |
| ATOM | 3290 | CG | ASN | A | 209 | 2.294 | 9.251 | 1.014 | 1.00 | 50.98 | C |
| ATOM | 3291 | OD1 | ASN | A | 209 | 3.237 | 8.778 | 1.628 | 1.00 | 61.21 | O |
| ATOM | 3292 | ND2 | ASN | A | 209 | 1.253 | 9.813 | 1.619 | 1.00 | 50.57 | N |
| ATOM | 3295 | C | ASN | A | 209 | 2.848 | 8.010 | −2.487 | 1.00 | 46.05 | C |
| ATOM | 3296 | O | ASN | A | 209 | 1.808 | 8.469 | −2.879 | 1.00 | 48.81 | O |
| ATOM | 3297 | N | GLY | A | 210 | 3.626 | 7.269 | −3.259 | 1.00 | 45.99 | N |
| ATOM | 3299 | CA | GLY | A | 210 | 3.168 | 6.840 | −4.550 | 1.00 | 39.71 | C |
| ATOM | 3302 | C | GLY | A | 210 | 3.521 | 7.817 | −5.634 | 1.00 | 44.70 | C |
| ATOM | 3303 | O | GLY | A | 210 | 3.523 | 7.484 | −6.817 | 1.00 | 52.94 | O |
| ATOM | 3304 | N | GLN | A | 211 | 3.832 | 9.040 | −5.259 | 1.00 | 48.14 | N |
| ATOM | 3306 | CA | GLN | A | 211 | 4.019 | 10.081 | −6.260 | 1.00 | 48.41 | C |
| ATOM | 3308 | CB | GLN | A | 211 | 3.696 | 11.450 | −5.650 | 1.00 | 47.90 | C |
| ATOM | 3311 | CG | GLN | A | 211 | 4.021 | 12.543 | −6.672 | 1.00 | 51.74 | C |
| ATOM | 3314 | CD | GLN | A | 211 | 3.464 | 13.875 | −6.326 | 1.00 | 48.04 | C |
| ATOM | 3315 | OE1 | GLN | A | 211 | 2.904 | 14.051 | −5.243 | 1.00 | 55.33 | O |
| ATOM | 3316 | NE2 | GLN | A | 211 | 3.609 | 14.829 | −7.242 | 1.00 | 43.28 | N |
| ATOM | 3319 | C | GLN | A | 211 | 5.442 | 10.132 | −6.820 | 1.00 | 44.36 | C |
| ATOM | 3320 | O | GLN | A | 211 | 6.328 | 10.489 | −6.090 | 1.00 | 40.60 | O |
| ATOM | 3321 | N | ASP | A | 212 | 5.638 | 9.788 | −8.096 | 1.00 | 48.43 | N |
| ATOM | 3323 | CA | ASP | A | 212 | 6.964 | 9.822 | −8.751 | 1.00 | 47.85 | C |
| ATOM | 3325 | CB | ASP | A | 212 | 6.864 | 9.644 | −10.277 | 1.00 | 49.02 | C |
| ATOM | 3328 | CG | ASP | A | 212 | 8.224 | 9.660 | −10.981 | 1.00 | 56.05 | C |
| ATOM | 3329 | OD1 | ASP | A | 212 | 9.140 | 10.315 | −10.471 | 1.00 | 65.69 | O |
| ATOM | 3330 | OD2 | ASP | A | 212 | 8.493 | 9.068 | −12.061 | 1.00 | 58.44 | O |
| ATOM | 3331 | C | ASP | A | 212 | 7.572 | 11.160 | −8.472 | 1.00 | 46.75 | C |
| ATOM | 3332 | O | ASP | A | 212 | 6.936 | 12.160 | −8.670 | 1.00 | 44.58 | O |
| ATOM | 3333 | N | LEU | A | 213 | 8.809 | 11.141 | −7.994 | 1.00 | 49.84 | N |
| ATOM | 3335 | CA | LEU | A | 213 | 9.594 | 12.334 | −7.729 | 1.00 | 49.00 | C |
| ATOM | 3337 | CB | LEU | A | 213 | 10.787 | 11.993 | −6.864 | 1.00 | 42.74 | C |
| ATOM | 3340 | CG | LEU | A | 213 | 11.249 | 13.197 | −6.091 | 1.00 | 38.09 | C |
| ATOM | 3342 | CD1 | LEU | A | 213 | 10.262 | 13.552 | −5.061 | 1.00 | 34.83 | C |
| ATOM | 3346 | CD2 | LEU | A | 213 | 12.551 | 12.820 | −5.448 | 1.00 | 44.89 | C |
| ATOM | 3350 | C | LEU | A | 213 | 10.102 | 12.901 | −9.036 | 1.00 | 51.86 | C |
| ATOM | 3351 | O | LEU | A | 213 | 10.526 | 14.046 | −9.102 | 1.00 | 60.24 | O |
| ATOM | 3352 | N | LYS | A | 214 | 10.103 | 12.091 | −10.077 | 1.00 | 50.59 | N |
| ATOM | 3354 | CA | LYS | A | 214 | 10.268 | 12.667 | −11.351 | 1.00 | 54.70 | C |
| ATOM | 3356 | CB | LYS | A | 214 | 9.120 | 13.673 | −11.542 | 1.00 | 58.84 | C |
| ATOM | 3359 | CG | LYS | A | 214 | 8.713 | 13.941 | −12.984 | 1.00 | 63.68 | C |
| ATOM | 3362 | CD | LYS | A | 214 | 7.904 | 12.796 | −13.625 | 1.00 | 65.33 | C |
| ATOM | 3365 | CE | LYS | A | 214 | 8.338 | 12.533 | −15.096 | 1.00 | 65.28 | C |
| ATOM | 3368 | NZ | LYS | A | 214 | 7.187 | 12.419 | −16.076 | 1.00 | 59.61 | N |
| ATOM | 3372 | C | LYS | A | 214 | 11.567 | 13.392 | −11.355 | 1.00 | 54.17 | C |
| ATOM | 3373 | O | LYS | A | 214 | 11.617 | 14.604 | −11.251 | 1.00 | 59.68 | O |
| ATOM | 3374 | N | MET | A | 215 | 12.632 | 12.628 | −11.413 | 1.00 | 56.61 | N |
| ATOM | 3376 | CA | MET | A | 215 | 13.932 | 13.165 | −11.741 | 1.00 | 57.04 | C |
| ATOM | 3378 | CB | MET | A | 215 | 14.810 | 13.356 | −10.511 | 1.00 | 57.19 | C |
| ATOM | 3381 | CG | MET | A | 215 | 14.273 | 14.458 | −9.634 | 1.00 | 60.76 | C |
| ATOM | 3384 | SD | MET | A | 215 | 15.283 | 14.900 | −8.238 | 1.00 | 63.39 | S |
| ATOM | 3385 | CE | MET | A | 215 | 16.644 | 15.670 | −9.212 | 1.00 | 63.56 | C |
| ATOM | 3389 | C | MET | A | 215 | 14.534 | 12.189 | −12.723 | 1.00 | 55.93 | C |
| ATOM | 3390 | O | MET | A | 215 | 14.070 | 11.083 | −12.896 | 1.00 | 51.96 | O |
| ATOM | 3391 | N | ASP | A | 216 | 15.562 | 12.614 | −13.410 | 1.00 | 58.57 | N |
| ATOM | 3393 | CA | ASP | A | 216 | 16.199 | 11.718 | −14.331 | 1.00 | 62.39 | C |
| ATOM | 3395 | CB | ASP | A | 216 | 17.142 | 12.500 | −15.225 | 1.00 | 64.34 | C |
| ATOM | 3398 | CG | ASP | A | 216 | 18.371 | 11.744 | −15.547 | 1.00 | 62.34 | C |
| ATOM | 3399 | OD1 | ASP | A | 216 | 18.562 | 10.659 | −14.958 | 1.00 | 64.91 | O |
| ATOM | 3400 | OD2 | ASP | A | 216 | 19.194 | 12.167 | −16.384 | 1.00 | 69.70 | O |
| ATOM | 3401 | C | ASP | A | 216 | 16.917 | 10.736 | −13.424 | 1.00 | 63.11 | C |
| ATOM | 3402 | O | ASP | A | 216 | 17.563 | 11.142 | −12.458 | 1.00 | 62.81 | O |
| ATOM | 3403 | N | CYS | A | 217 | 16.824 | 9.446 | −13.708 | 1.00 | 64.18 | N |
| ATOM | 3405 | CA | CYS | A | 217 | 17.358 | 8.520 | −12.735 | 1.00 | 66.46 | C |
| ATOM | 3407 | CB | CYS | A | 217 | 17.022 | 7.056 | −13.051 | 1.00 | 66.11 | C |
| ATOM | 3410 | SG | CYS | A | 217 | 17.799 | 6.334 | −14.479 | 1.00 | 72.28 | S |
| ATOM | 3411 | C | CYS | A | 217 | 18.822 | 8.755 | −12.482 | 1.00 | 65.55 | C |
| ATOM | 3412 | O | CYS | A | 217 | 19.283 | 8.527 | −11.381 | 1.00 | 69.85 | O |
| ATOM | 3413 | N | LYS | A | 218 | 19.547 | 9.255 | −13.474 | 1.00 | 68.81 | N |
| ATOM | 3415 | CA | LYS | A | 218 | 20.992 | 9.436 | −13.327 | 1.00 | 70.13 | C |
| ATOM | 3417 | CB | LYS | A | 218 | 21.618 | 10.160 | −14.554 | 1.00 | 73.78 | C |
| ATOM | 3420 | CG | LYS | A | 218 | 22.335 | 9.229 | −15.609 | 1.00 | 76.00 | C |
| ATOM | 3423 | CD | LYS | A | 218 | 23.033 | 9.987 | −16.810 | 1.00 | 74.40 | C |
| ATOM | 3426 | CE | LYS | A | 218 | 23.953 | 9.050 | −17.717 | 1.00 | 75.92 | C |
| ATOM | 3429 | NZ | LYS | A | 218 | 23.299 | 7.995 | −18.636 | 1.00 | 68.28 | N |
| ATOM | 3433 | C | LYS | A | 218 | 21.288 | 10.204 | −12.055 | 1.00 | 66.58 | C |
| ATOM | 3434 | O | LYS | A | 218 | 22.340 | 10.059 | −11.456 | 1.00 | 68.40 | O |
| ATOM | 3435 | N | GLU | A | 219 | 20.347 | 11.014 | −11.621 | 1.00 | 61.73 | N |
| ATOM | 3437 | CA | GLU | A | 219 | 20.604 | 11.871 | −10.482 | 1.00 | 61.25 | C |
| ATOM | 3439 | CB | GLU | A | 219 | 19.634 | 13.041 | −10.528 | 1.00 | 60.32 | C |
| ATOM | 3442 | CG | GLU | A | 219 | 19.746 | 13.887 | −11.782 | 1.00 | 65.08 | C |
| ATOM | 3445 | CD | GLU | A | 219 | 20.977 | 14.781 | −11.827 | 1.00 | 65.35 | C |
| ATOM | 3446 | OE1 | GLU | A | 219 | 21.905 | 14.598 | −10.998 | 1.00 | 62.03 | O |
| ATOM | 3447 | OE2 | GLU | A | 219 | 21.001 | 15.673 | −12.717 | 1.00 | 68.30 | O |
| ATOM | 3448 | C | GLU | A | 219 | 20.493 | 11.211 | −9.113 | 1.00 | 60.55 | C |
| ATOM | 3449 | O | GLU | A | 219 | 20.976 | 11.755 | −8.113 | 1.00 | 60.04 | O |
| ATOM | 3450 | N | TYR | A | 220 | 19.848 | 10.055 | −9.051 | 1.00 | 58.43 | N |
| ATOM | 3452 | CA | TYR | A | 220 | 19.599 | 9.427 | −7.771 | 1.00 | 54.98 | C |
| ATOM | 3454 | CB | TYR | A | 220 | 18.533 | 8.374 | −7.903 | 1.00 | 56.27 | C |
| ATOM | 3457 | CG | TYR | A | 220 | 17.175 | 8.915 | −8.250 | 1.00 | 51.16 | C |
| ATOM | 3458 | CD1 | TYR | A | 220 | 16.460 | 8.385 | −9.284 | 1.00 | 47.88 | C |
| ATOM | 3460 | CE1 | TYR | A | 220 | 15.232 | 8.859 | −9.601 | 1.00 | 53.59 | C |
| ATOM | 3462 | CZ | TYR | A | 220 | 14.696 | 9.898 | −8.866 | 1.00 | 54.76 | C |
| ATOM | 3463 | OH | TYR | A | 220 | 13.443 | 10.384 | −9.181 | 1.00 | 59.59 | O |
| ATOM | 3465 | CE2 | TYR | A | 220 | 15.390 | 10.431 | −7.834 | 1.00 | 45.89 | C |
| ATOM | 3467 | CD2 | TYR | A | 220 | 16.619 | 9.943 | −7.529 | 1.00 | 48.33 | C |
| ATOM | 3469 | C | TYR | A | 220 | 20.844 | 8.778 | −7.324 | 1.00 | 55.05 | C |
| ATOM | 3470 | O | TYR | A | 220 | 21.101 | 8.684 | −6.135 | 1.00 | 58.11 | O |
| ATOM | 3471 | N | ASN | A | 221 | 21.616 | 8.340 | −8.310 | 1.00 | 56.80 | N |
| ATOM | 3473 | CA | ASN | A | 221 | 22.886 | 7.658 | −8.110 | 1.00 | 56.10 | C |
| ATOM | 3475 | CB | ASN | A | 221 | 22.874 | 6.380 | −8.928 | 1.00 | 51.38 | C |
| ATOM | 3478 | CG | ASN | A | 221 | 21.838 | 5.430 | −8.435 | 1.00 | 54.95 | C |
| ATOM | 3479 | OD1 | ASN | A | 221 | 21.923 | 4.970 | −7.294 | 1.00 | 53.01 | O |
| ATOM | 3480 | ND2 | ASN | A | 221 | 20.830 | 5.136 | −9.272 | 1.00 | 50.76 | N |
| ATOM | 3483 | C | ASN | A | 221 | 24.068 | 8.511 | −8.521 | 1.00 | 59.72 | C |
| ATOM | 3484 | O | ASN | A | 221 | 25.123 | 7.989 | −8.925 | 1.00 | 63.63 | O |
| ATOM | 3485 | N | TYR | A | 222 | 23.909 | 9.823 | −8.393 | 1.00 | 63.40 | N |
| ATOM | 3487 | CA | TYR | A | 222 | 24.919 | 10.707 | −8.917 | 1.00 | 63.06 | C |
| ATOM | 3489 | CB | TYR | A | 222 | 24.506 | 12.178 | −9.007 | 1.00 | 68.66 | C |
| ATOM | 3492 | CG | TYR | A | 222 | 25.696 | 13.048 | −9.334 | 1.00 | 67.88 | C |
| ATOM | 3493 | CD1 | TYR | A | 222 | 26.577 | 12.678 | −10.333 | 1.00 | 72.26 | C |
| ATOM | 3495 | CE1 | TYR | A | 222 | 27.666 | 13.446 | −10.632 | 1.00 | 77.81 | C |
| ATOM | 3497 | CZ | TYR | A | 222 | 27.889 | 14.606 | −9.925 | 1.00 | 79.75 | C |
| ATOM | 3498 | OH | TYR | A | 222 | 28.992 | 15.374 | −10.239 | 1.00 | 83.62 | O |
| ATOM | 3500 | CE2 | TYR | A | 222 | 27.018 | 14.993 | −8.917 | 1.00 | 75.27 | C |
| ATOM | 3502 | CD2 | TYR | A | 222 | 25.942 | 14.216 | −8.632 | 1.00 | 69.85 | C |
| ATOM | 3504 | C | TYR | A | 222 | 26.139 | 10.534 | −8.091 | 1.00 | 61.93 | C |
| ATOM | 3505 | O | TYR | A | 222 | 26.223 | 10.993 | −6.934 | 1.00 | 51.08 | O |
| ATOM | 3506 | N | ASP | A | 223 | 27.055 | 9.829 | −8.771 | 1.00 | 65.98 | N |
| ATOM | 3508 | CA | ASP | A | 223 | 28.389 | 9.416 | −8.349 | 1.00 | 58.22 | C |
| ATOM | 3510 | CB | ASP | A | 223 | 29.017 | 10.429 | −7.382 | 1.00 | 58.25 | C |
| ATOM | 3513 | CG | ASP | A | 223 | 29.709 | 9.795 | −6.239 | 1.00 | 64.15 | C |
| ATOM | 3514 | OD1 | ASP | A | 223 | 28.983 | 9.249 | −5.377 | 1.00 | 75.60 | O |
| ATOM | 3515 | OD2 | ASP | A | 223 | 30.956 | 9.791 | −6.110 | 1.00 | 56.47 | O |
| ATOM | 3516 | C | ASP | A | 223 | 28.238 | 7.959 | −7.885 | 1.00 | 56.27 | C |
| ATOM | 3517 | O | ASP | A | 223 | 28.790 | 7.052 | −8.514 | 1.00 | 48.60 | O |
| ATOM | 3518 | N | LYS | A | 224 | 27.463 | 7.710 | −6.829 | 1.00 | 60.43 | N |
| ATOM | 3520 | CA | LYS | A | 224 | 27.203 | 6.317 | −6.389 | 1.00 | 58.39 | C |
| ATOM | 3522 | CB | LYS | A | 224 | 28.440 | 5.692 | −5.784 | 1.00 | 55.03 | C |
| ATOM | 3525 | CG | LYS | A | 224 | 28.722 | 6.175 | −4.403 | 1.00 | 53.98 | C |
| ATOM | 3528 | CD | LYS | A | 224 | 30.039 | 5.591 | −3.910 | 1.00 | 51.13 | C |
| ATOM | 3531 | CE | LYS | A | 224 | 30.576 | 6.430 | −2.777 | 1.00 | 50.78 | C |
| ATOM | 3534 | NZ | LYS | A | 224 | 30.830 | 7.806 | −3.279 | 1.00 | 52.27 | N |
| ATOM | 3538 | C | LYS | A | 224 | 26.093 | 6.132 | −5.382 | 1.00 | 57.12 | C |
| ATOM | 3539 | O | LYS | A | 224 | 25.621 | 7.085 | −4.735 | 1.00 | 59.55 | O |
| ATOM | 3540 | N | SER | A | 225 | 25.697 | 4.870 | −5.246 | 1.00 | 56.45 | N |
| ATOM | 3542 | CA | SER | A | 225 | 24.636 | 4.461 | −4.316 | 1.00 | 52.86 | C |
| ATOM | 3544 | CB | SER | A | 225 | 23.338 | 4.274 | −5.076 | 1.00 | 50.03 | C |
| ATOM | 3547 | OG | SER | A | 225 | 22.661 | 5.516 | −5.099 | 1.00 | 52.69 | O |
| ATOM | 3549 | C | SER | A | 225 | 25.040 | 3.197 | −3.570 | 1.00 | 48.24 | C |
| ATOM | 3550 | O | SER | A | 225 | 25.422 | 2.202 | −4.180 | 1.00 | 48.80 | O |
| ATOM | 3551 | N | ILE | A | 226 | 24.969 | 3.241 | −2.244 | 1.00 | 44.93 | N |
| ATOM | 3553 | CA | ILE | A | 226 | 25.441 | 2.125 | −1.454 | 1.00 | 37.58 | C |
| ATOM | 3555 | CB | ILE | A | 226 | 26.875 | 2.379 | −1.017 | 1.00 | 39.22 | C |
| ATOM | 3557 | CG1 | ILE | A | 226 | 26.923 | 3.507 | −0.005 | 1.00 | 37.98 | C |
| ATOM | 3560 | CD1 | ILE | A | 226 | 28.301 | 3.756 | 0.555 | 1.00 | 34.23 | C |
| ATOM | 3564 | CG2 | ILE | A | 226 | 27.756 | 2.690 | −2.212 | 1.00 | 38.02 | C |
| ATOM | 3568 | C | ILE | A | 226 | 24.632 | 1.817 | −0.239 | 1.00 | 38.95 | C |
| ATOM | 3569 | O | ILE | A | 226 | 23.774 | 2.567 | 0.185 | 1.00 | 47.23 | O |
| ATOM | 3570 | N | VAL | A | 227 | 24.920 | 0.635 | 0.310 | 1.00 | 40.07 | N |
| ATOM | 3572 | CA | VAL | A | 227 | 24.282 | 0.137 | 1.528 | 1.00 | 39.95 | C |
| ATOM | 3574 | CB | VAL | A | 227 | 23.835 | −1.340 | 1.378 | 1.00 | 36.39 | C |
| ATOM | 3576 | CG1 | VAL | A | 227 | 23.223 | −1.863 | 2.677 | 1.00 | 42.20 | C |
| ATOM | 3580 | CG2 | VAL | A | 227 | 22.852 | −1.446 | 0.279 | 1.00 | 32.66 | C |
| ATOM | 3584 | C | VAL | A | 227 | 25.298 | 0.175 | 2.644 | 1.00 | 41.52 | C |
| ATOM | 3585 | O | VAL | A | 227 | 26.400 | −0.407 | 2.485 | 1.00 | 31.57 | O |
| ATOM | 3586 | N | ASP | A | 228 | 24.951 | 0.813 | 3.768 | 1.00 | 42.83 | N |
| ATOM | 3588 | CA | ASP | A | 228 | 25.934 | 0.901 | 4.892 | 1.00 | 44.00 | C |
| ATOM | 3590 | CB | ASP | A | 228 | 26.926 | 2.001 | 4.665 | 1.00 | 47.22 | C |
| ATOM | 3593 | CG | ASP | A | 228 | 27.484 | 2.542 | 5.943 | 1.00 | 55.34 | C |
| ATOM | 3594 | OD1 | ASP | A | 228 | 27.322 | 1.886 | 6.990 | 1.00 | 60.37 | O |
| ATOM | 3595 | OD2 | ASP | A | 228 | 28.093 | 3.631 | 6.003 | 1.00 | 65.03 | O |
| ATOM | 3596 | C | ASP | A | 228 | 25.364 | 1.037 | 6.287 | 1.00 | 43.99 | C |
| ATOM | 3597 | O | ASP | A | 228 | 24.829 | 2.037 | 6.705 | 1.00 | 43.43 | O |
| ATOM | 3598 | N | SER | A | 229 | 25.535 | −0.057 | 6.977 | 1.00 | 45.99 | N |
| ATOM | 3600 | CA | SER | A | 229 | 25.113 | −0.268 | 8.307 | 1.00 | 43.77 | C |
| ATOM | 3602 | CB | SER | A | 229 | 25.400 | −1.715 | 8.589 | 1.00 | 43.80 | C |
| ATOM | 3605 | OG | SER | A | 229 | 26.786 | −1.836 | 8.462 | 1.00 | 43.49 | O |
| ATOM | 3607 | C | SER | A | 229 | 25.987 | 0.478 | 9.228 | 1.00 | 39.36 | C |
| ATOM | 3608 | O | SER | A | 229 | 25.806 | 0.377 | 10.394 | 1.00 | 35.35 | O |
| ATOM | 3609 | N | GLY | A | 230 | 26.982 | 1.181 | 8.705 | 1.00 | 40.62 | N |
| ATOM | 3611 | CA | GLY | A | 230 | 27.805 | 2.105 | 9.509 | 1.00 | 37.16 | C |
| ATOM | 3614 | C | GLY | A | 230 | 27.388 | 3.543 | 9.319 | 1.00 | 38.50 | C |
| ATOM | 3615 | O | GLY | A | 230 | 28.040 | 4.500 | 9.740 | 1.00 | 38.84 | O |
| ATOM | 3616 | N | THR | A | 231 | 26.265 | 3.690 | 8.657 | 1.00 | 36.83 | N |
| ATOM | 3618 | CA | THR | A | 231 | 25.672 | 4.981 | 8.477 | 1.00 | 36.29 | C |
| ATOM | 3620 | CB | THR | A | 231 | 25.795 | 5.339 | 7.037 | 1.00 | 36.20 | C |
| ATOM | 3622 | OG1 | THR | A | 231 | 27.141 | 5.728 | 6.829 | 1.00 | 32.63 | O |
| ATOM | 3624 | CG2 | THR | A | 231 | 24.980 | 6.570 | 6.709 | 1.00 | 33.65 | C |
| ATOM | 3628 | C | THR | A | 231 | 24.231 | 4.977 | 8.968 | 1.00 | 36.96 | C |
| ATOM | 3629 | O | THR | A | 231 | 23.479 | 4.036 | 8.702 | 1.00 | 36.51 | O |
| ATOM | 3630 | N | THR | A | 232 | 23.868 | 6.048 | 9.672 | 1.00 | 39.63 | N |
| ATOM | 3632 | CA | THR | A | 232 | 22.586 | 6.197 | 10.378 | 1.00 | 44.48 | C |
| ATOM | 3634 | CB | THR | A | 232 | 22.776 | 7.362 | 11.370 | 1.00 | 47.87 | C |
| ATOM | 3636 | OG1 | THR | A | 232 | 23.819 | 7.053 | 12.305 | 1.00 | 58.32 | O |
| ATOM | 3638 | CG2 | THR | A | 232 | 21.553 | 7.610 | 12.240 | 1.00 | 52.16 | C |
| ATOM | 3642 | C | THR | A | 232 | 21.384 | 6.554 | 9.507 | 1.00 | 46.65 | C |
| ATOM | 3643 | O | THR | A | 232 | 20.315 | 5.954 | 9.593 | 1.00 | 48.65 | O |
| ATOM | 3644 | N | ASN | A | 233 | 21.590 | 7.547 | 8.663 | 1.00 | 47.89 | N |
| ATOM | 3646 | CA | ASN | A | 233 | 20.523 | 8.231 | 7.983 | 1.00 | 47.43 | C |
| ATOM | 3648 | CB | ASN | A | 233 | 20.878 | 9.715 | 7.971 | 1.00 | 48.12 | C |
| ATOM | 3651 | CG | ASN | A | 233 | 20.435 | 10.435 | 9.198 | 1.00 | 43.02 | C |
| ATOM | 3652 | OD1 | ASN | A | 233 | 20.365 | 9.883 | 10.276 | 1.00 | 38.96 | O |
| ATOM | 3653 | ND2 | ASN | A | 233 | 20.145 | 11.703 | 9.035 | 1.00 | 50.70 | N |
| ATOM | 3656 | C | ASN | A | 233 | 20.451 | 7.803 | 6.572 | 1.00 | 48.67 | C |
| ATOM | 3657 | O | ASN | A | 233 | 21.360 | 7.093 | 6.131 | 1.00 | 51.81 | O |
| ATOM | 3658 | N | LEU | A | 234 | 19.403 | 8.234 | 5.856 | 1.00 | 45.12 | N |
| ATOM | 3660 | CA | LEU | A | 234 | 19.333 | 7.999 | 4.413 | 1.00 | 46.15 | C |
| ATOM | 3662 | CB | LEU | A | 234 | 17.912 | 7.787 | 3.895 | 1.00 | 46.39 | C |
| ATOM | 3665 | CG | LEU | A | 234 | 17.655 | 7.385 | 2.424 | 1.00 | 46.15 | C |
| ATOM | 3667 | CD1 | LEU | A | 234 | 16.562 | 8.293 | 1.862 | 1.00 | 47.55 | C |
| ATOM | 3671 | CD2 | LEU | A | 234 | 18.868 | 7.415 | 1.522 | 1.00 | 47.77 | C |
| ATOM | 3675 | C | LEU | A | 234 | 19.842 | 9.286 | 3.867 | 1.00 | 45.80 | C |
| ATOM | 3676 | O | LEU | A | 234 | 19.245 | 10.301 | 4.146 | 1.00 | 51.40 | O |
| ATOM | 3677 | N | ARG | A | 235 | 20.940 | 9.231 | 3.104 | 1.00 | 47.98 | N |
| ATOM | 3679 | CA | ARG | A | 235 | 21.555 | 10.393 | 2.452 | 1.00 | 41.53 | C |
| ATOM | 3681 | CB | ARG | A | 235 | 23.058 | 10.339 | 2.514 | 1.00 | 40.87 | C |
| ATOM | 3684 | CG | ARG | A | 235 | 23.653 | 9.849 | 3.761 | 1.00 | 41.40 | C |
| ATOM | 3687 | CD | ARG | A | 235 | 23.645 | 10.825 | 4.775 | 1.00 | 46.10 | C |
| ATOM | 3690 | NE | ARG | A | 235 | 24.981 | 11.103 | 5.305 | 1.00 | 52.10 | N |
| ATOM | 3692 | CZ | ARG | A | 235 | 25.469 | 12.342 | 5.380 | 1.00 | 54.49 | C |
| ATOM | 3693 | NH1 | ARG | A | 235 | 26.649 | 12.577 | 5.870 | 1.00 | 51.49 | N |
| ATOM | 3696 | NH2 | ARG | A | 235 | 24.751 | 13.366 | 4.949 | 1.00 | 62.47 | N |
| ATOM | 3699 | C | ARG | A | 235 | 21.284 | 10.362 | 0.984 | 1.00 | 42.47 | C |
| ATOM | 3700 | O | ARG | A | 235 | 21.322 | 9.234 | 0.354 | 1.00 | 35.71 | O |
| ATOM | 3701 | N | LEU | A | 236 | 21.054 | 11.575 | 0.446 | 1.00 | 42.38 | N |
| ATOM | 3703 | CA | LEU | A | 236 | 20.755 | 11.710 | −0.959 | 1.00 | 44.15 | C |
| ATOM | 3705 | CB | LEU | A | 236 | 19.287 | 12.033 | −1.112 | 1.00 | 46.30 | C |
| ATOM | 3708 | CG | LEU | A | 236 | 18.420 | 11.070 | −0.325 | 1.00 | 46.08 | C |
| ATOM | 3710 | CD1 | LEU | A | 236 | 17.143 | 11.742 | 0.192 | 1.00 | 50.37 | C |
| ATOM | 3714 | CD2 | LEU | A | 236 | 18.143 | 9.851 | −1.201 | 1.00 | 46.39 | C |
| ATOM | 3718 | C | LEU | A | 236 | 21.574 | 12.774 | −1.606 | 1.00 | 46.18 | C |
| ATOM | 3719 | O | LEU | A | 236 | 21.974 | 13.760 | −1.003 | 1.00 | 56.50 | O |
| ATOM | 3720 | N | PRO | A | 237 | 21.861 | 12.556 | −2.850 | 1.00 | 43.50 | N |
| ATOM | 3721 | CA | PRO | A | 237 | 22.474 | 13.586 | −3.669 | 1.00 | 48.09 | C |
| ATOM | 3723 | CB | PRO | A | 237 | 22.378 | 13.003 | −5.075 | 1.00 | 50.44 | C |
| ATOM | 3726 | CG | PRO | A | 237 | 22.263 | 11.517 | −4.873 | 1.00 | 47.91 | C |
| ATOM | 3729 | CD | PRO | A | 237 | 21.677 | 11.287 | −3.541 | 1.00 | 43.62 | C |
| ATOM | 3732 | C | PRO | A | 237 | 21.657 | 14.854 | −3.533 | 1.00 | 47.59 | C |
| ATOM | 3733 | O | PRO | A | 237 | 20.443 | 14.793 | −3.621 | 1.00 | 56.42 | O |
| ATOM | 3734 | N | LYS | A | 238 | 22.324 | 15.977 | −3.340 | 1.00 | 54.44 | N |
| ATOM | 3736 | CA | LYS | A | 238 | 21.710 | 17.262 | −2.966 | 1.00 | 59.43 | C |
| ATOM | 3738 | CB | LYS | A | 238 | 22.759 | 18.345 | −3.006 | 1.00 | 65.19 | C |
| ATOM | 3741 | CG | LYS | A | 238 | 22.458 | 19.459 | −2.056 | 1.00 | 68.51 | C |
| ATOM | 3744 | CD | LYS | A | 238 | 21.616 | 20.524 | −2.702 | 1.00 | 69.31 | C |
| ATOM | 3747 | CE | LYS | A | 238 | 21.206 | 21.491 | −1.626 | 1.00 | 74.63 | C |
| ATOM | 3750 | NZ | LYS | A | 238 | 21.148 | 20.760 | −0.296 | 1.00 | 72.38 | N |
| ATOM | 3754 | C | LYS | A | 238 | 20.530 | 17.740 | −3.770 | 1.00 | 60.04 | C |
| ATOM | 3755 | O | LYS | A | 238 | 19.535 | 18.188 | −3.218 | 1.00 | 62.70 | O |
| ATOM | 3756 | N | LYS | A | 239 | 20.645 | 17.681 | −5.078 | 1.00 | 61.99 | N |
| ATOM | 3758 | CA | LYS | A | 239 | 19.502 | 17.942 | −5.889 | 1.00 | 62.06 | C |
| ATOM | 3760 | CB | LYS | A | 239 | 19.803 | 17.627 | −7.373 | 1.00 | 65.93 | C |
| ATOM | 3763 | CG | LYS | A | 239 | 19.794 | 18.830 | −8.411 | 1.00 | 69.24 | C |
| ATOM | 3766 | CD | LYS | A | 239 | 21.071 | 18.808 | −9.350 | 1.00 | 71.82 | C |
| ATOM | 3769 | CE | LYS | A | 239 | 20.782 | 18.915 | −10.877 | 1.00 | 73.55 | C |
| ATOM | 3772 | NZ | LYS | A | 239 | 21.914 | 18.411 | −11.760 | 1.00 | 70.78 | N |
| ATOM | 3776 | C | LYS | A | 239 | 18.444 | 16.999 | −5.285 | 1.00 | 64.24 | C |
| ATOM | 3777 | O | LYS | A | 239 | 17.406 | 17.455 | −4.781 | 1.00 | 68.73 | O |
| ATOM | 3778 | N | VAL | A | 240 | 18.705 | 15.691 | −5.278 | 1.00 | 58.91 | N |
| ATOM | 3780 | CA | VAL | A | 240 | 17.635 | 14.751 | −4.914 | 1.00 | 62.19 | C |
| ATOM | 3782 | CB | VAL | A | 240 | 18.864 | 13.284 | −4.909 | 1.00 | 65.97 | C |
| ATOM | 3784 | CG1 | VAL | A | 240 | 16.867 | 12.392 | −4.847 | 1.00 | 66.12 | C |
| ATOM | 3788 | CG2 | VAL | A | 240 | 18.913 | 12.947 | −6.147 | 1.00 | 68.39 | C |
| ATOM | 3792 | C | VAL | A | 240 | 17.034 | 15.079 | −3.556 | 1.00 | 58.68 | C |
| ATOM | 3793 | O | VAL | A | 240 | 15.811 | 15.151 | −3.379 | 1.00 | 54.40 | O |
| ATOM | 3794 | N | PHE | A | 241 | 17.906 | 15.282 | −2.587 | 1.00 | 56.41 | N |
| ATOM | 3796 | CA | PHE | A | 241 | 17.445 | 15.677 | −1.278 | 1.00 | 52.63 | C |
| ATOM | 3798 | CB | PHE | A | 241 | 18.635 | 15.966 | −0.376 | 1.00 | 50.48 | C |
| ATOM | 3801 | CG | PHE | A | 241 | 18.276 | 16.566 | 0.957 | 1.00 | 50.54 | C |
| ATOM | 3802 | CD1 | PHE | A | 241 | 17.615 | 15.854 | 1.945 | 1.00 | 53.71 | C |
| ATOM | 3804 | CE1 | PHE | A | 241 | 17.303 | 16.447 | 3.181 | 1.00 | 54.61 | C |
| ATOM | 3806 | CZ | PHE | A | 241 | 17.662 | 17.775 | 3.427 | 1.00 | 55.24 | C |
| ATOM | 3808 | CE2 | PHE | A | 241 | 18.322 | 18.488 | 2.440 | 1.00 | 55.17 | C |
| ATOM | 3810 | CD2 | PHE | A | 241 | 18.624 | 17.870 | 1.219 | 1.00 | 57.01 | C |
| ATOM | 3812 | C | PHE | A | 241 | 16.561 | 16.885 | −1.477 | 1.00 | 51.84 | C |
| ATOM | 3813 | O | PHE | A | 241 | 15.439 | 16.937 | −0.971 | 1.00 | 47.71 | O |
| ATOM | 3814 | N | GLU | A | 242 | 17.031 | 17.857 | −2.245 | 1.00 | 53.89 | N |
| ATOM | 3816 | CA | GLU | A | 242 | 16.239 | 19.085 | −2.353 | 1.00 | 56.53 | C |
| ATOM | 3818 | CB | GLU | A | 242 | 16.797 | 20.110 | −3.362 | 1.00 | 59.79 | C |
| ATOM | 3821 | CG | GLU | A | 242 | 17.432 | 21.353 | −2.697 | 1.00 | 66.19 | C |
| ATOM | 3824 | CD | GLU | A | 242 | 17.130 | 21.454 | −1.185 | 1.00 | 74.63 | C |
| ATOM | 3825 | OE1 | GLU | A | 242 | 15.931 | 21.492 | −0.789 | 1.00 | 79.35 | O |
| ATOM | 3826 | OE2 | GLU | A | 242 | 18.091 | 21.487 | −0.372 | 1.00 | 76.40 | O |
| ATOM | 3827 | C | GLU | A | 242 | 14.790 | 18.768 | −2.638 | 1.00 | 53.63 | C |
| ATOM | 3828 | O | GLU | A | 242 | 13.937 | 19.203 | −1.880 | 1.00 | 45.24 | O |
| ATOM | 3829 | N | ALA | A | 243 | 14.536 | 17.997 | −3.709 | 1.00 | 52.72 | N |
| ATOM | 3831 | CA | ALA | A | 243 | 13.180 | 17.599 | −4.140 | 1.00 | 50.49 | C |
| ATOM | 3833 | CB | ALA | A | 243 | 13.258 | 16.838 | −5.428 | 1.00 | 51.48 | C |
| ATOM | 3837 | C | ALA | A | 243 | 12.383 | 16.781 | −3.120 | 1.00 | 51.02 | C |
| ATOM | 3838 | O | ALA | A | 243 | 11.259 | 17.157 | −2.763 | 1.00 | 54.64 | O |
| ATOM | 3839 | N | ALA | A | 244 | 12.949 | 15.670 | −2.653 | 1.00 | 46.51 | N |
| ATOM | 3841 | CA | ALA | A | 244 | 12.262 | 14.833 | −1.670 | 1.00 | 41.75 | C |
| ATOM | 3843 | CB | ALA | A | 244 | 13.173 | 13.694 | −1.156 | 1.00 | 37.69 | C |
| ATOM | 3847 | C | ALA | A | 244 | 11.792 | 15.630 | −0.490 | 1.00 | 44.76 | C |
| ATOM | 3848 | O | ALA | A | 244 | 10.658 | 15.496 | −0.084 | 1.00 | 47.71 | O |
| ATOM | 3849 | N | VAL | A | 245 | 12.646 | 16.470 | 0.083 | 1.00 | 52.14 | N |
| ATOM | 3851 | CA | VAL | A | 245 | 12.279 | 17.094 | 1.348 | 1.00 | 53.69 | C |
| ATOM | 3853 | CB | VAL | A | 245 | 13.325 | 18.100 | 1.854 | 1.00 | 53.93 | C |
| ATOM | 3855 | CG1 | VAL | A | 245 | 12.824 | 18.785 | 3.121 | 1.00 | 53.73 | C |
| ATOM | 3859 | CG2 | VAL | A | 245 | 14.659 | 17.433 | 2.106 | 1.00 | 58.55 | C |
| ATOM | 3863 | C | VAL | A | 245 | 10.976 | 17.810 | 1.141 | 1.00 | 52.68 | C |
| ATOM | 3864 | O | VAL | A | 245 | 10.072 | 17.775 | 1.976 | 1.00 | 57.49 | O |
| ATOM | 3865 | N | LYS | A | 246 | 10.904 | 18.444 | −0.015 | 1.00 | 49.32 | N |
| ATOM | 3867 | CA | LYS | A | 246 | 9.816 | 19.327 | −0.367 | 1.00 | 54.20 | C |
| ATOM | 3869 | CB | LYS | A | 246 | 10.200 | 19.988 | −1.684 | 1.00 | 58.55 | C |
| ATOM | 3872 | CG | LYS | A | 246 | 11.763 | 19.868 | −1.943 | 1.00 | 63.23 | C |
| ATOM | 3875 | CD | LYS | A | 246 | 12.640 | 21.041 | −1.337 | 1.00 | 70.71 | C |
| ATOM | 3878 | CE | LYS | A | 246 | 13.228 | 20.765 | 0.055 | 1.00 | 70.11 | C |
| ATOM | 3881 | NZ | LYS | A | 246 | 13.337 | 22.015 | 0.860 | 1.00 | 70.79 | N |
| ATOM | 3885 | C | LYS | A | 246 | 8.550 | 18.497 | −0.458 | 1.00 | 56.50 | C |
| ATOM | 3886 | O | LYS | A | 246 | 7.635 | 18.653 | 0.345 | 1.00 | 62.61 | O |
| ATOM | 3887 | N | SER | A | 247 | 8.510 | 17.574 | −1.417 | 1.00 | 53.80 | N |
| ATOM | 3889 | CA | SER | A | 247 | 7.381 | 16.674 | −1.534 | 1.00 | 45.07 | C |
| ATOM | 3891 | CB | SER | A | 247 | 7.699 | 15.537 | −2.496 | 1.00 | 45.02 | C |
| ATOM | 3894 | OG | SER | A | 247 | 6.636 | 14.587 | −2.462 | 1.00 | 43.14 | O |
| ATOM | 3896 | C | SER | A | 247 | 7.010 | 16.123 | −0.162 | 1.00 | 38.07 | C |
| ATOM | 3897 | O | SER | A | 247 | 5.842 | 15.925 | 0.137 | 1.00 | 47.02 | O |
| ATOM | 3898 | N | ILE | A | 248 | 8.008 | 15.906 | 0.685 | 1.00 | 35.51 | N |
| ATOM | 3900 | CA | ILE | A | 248 | 7.738 | 15.379 | 2.012 | 1.00 | 44.04 | C |
| ATOM | 3902 | CB | ILE | A | 248 | 9.068 | 14.787 | 2.672 | 1.00 | 45.65 | C |
| ATOM | 3904 | CG1 | ILE | A | 248 | 9.363 | 13.394 | 2.080 | 1.00 | 38.16 | C |
| ATOM | 3907 | CD1 | ILE | A | 248 | 10.760 | 12.863 | 2.337 | 1.00 | 33.98 | C |
| ATOM | 3911 | CG2 | ILE | A | 248 | 8.970 | 14.588 | 4.187 | 1.00 | 46.92 | C |
| ATOM | 3915 | C | ILE | A | 248 | 6.955 | 16.401 | 2.846 | 1.00 | 51.15 | C |
| ATOM | 3916 | O | ILE | A | 248 | 6.052 | 16.024 | 3.606 | 1.00 | 56.50 | O |
| ATOM | 3917 | N | LYS | A | 249 | 7.281 | 17.689 | 2.671 | 1.00 | 53.92 | N |
| ATOM | 3919 | CA | LYS | A | 249 | 6.627 | 18.796 | 3.389 | 1.00 | 54.59 | C |
| ATOM | 3921 | CB | LYS | A | 249 | 7.303 | 20.147 | 3.071 | 1.00 | 58.74 | C |
| ATOM | 3924 | CG | LYS | A | 249 | 8.585 | 20.554 | 3.850 | 1.00 | 58.89 | C |
| ATOM | 3927 | CD | LYS | A | 249 | 9.099 | 21.880 | 3.249 | 1.00 | 58.57 | C |
| ATOM | 3930 | CE | LYS | A | 249 | 10.483 | 22.330 | 3.774 | 1.00 | 61.39 | C |
| ATOM | 3933 | NZ | LYS | A | 249 | 11.350 | 22.849 | 2.647 | 1.00 | 57.83 | N |
| ATOM | 3937 | C | LYS | A | 249 | 5.177 | 18.935 | 2.964 | 1.00 | 54.92 | C |
| ATOM | 3938 | O | LYS | A | 249 | 4.246 | 18.932 | 3.790 | 1.00 | 54.00 | O |
| ATOM | 3939 | N | ALA | A | 250 | 5.003 | 19.083 | 1.656 | 1.00 | 49.70 | N |
| ATOM | 3941 | CA | ALA | A | 250 | 3.688 | 19.218 | 1.060 | 1.00 | 52.00 | C |
| ATOM | 3943 | CB | ALA | A | 250 | 3.786 | 19.096 | −0.465 | 1.00 | 53.40 | C |
| ATOM | 3947 | C | ALA | A | 250 | 2.773 | 18.163 | 1.636 | 1.00 | 53.09 | C |
| ATOM | 3948 | O | ALA | A | 250 | 1.602 | 18.407 | 1.922 | 1.00 | 60.66 | O |
| ATOM | 3949 | N | ALA | A | 251 | 3.344 | 16.980 | 1.805 | 1.00 | 57.05 | N |
| ATOM | 3951 | CA | ALA | A | 251 | 2.639 | 15.818 | 2.338 | 1.00 | 55.70 | C |
| ATOM | 3953 | CB | ALA | A | 251 | 3.420 | 14.558 | 1.986 | 1.00 | 53.52 | C |
| ATOM | 3957 | C | ALA | A | 251 | 2.404 | 15.877 | 3.856 | 1.00 | 58.05 | C |
| ATOM | 3958 | O | ALA | A | 251 | 1.461 | 15.323 | 4.372 | 1.00 | 62.89 | O |
| ATOM | 3959 | N | SER | A | 252 | 3.281 | 16.529 | 4.590 | 1.00 | 59.77 | N |
| ATOM | 3961 | CA | SER | A | 252 | 3.147 | 16.572 | 6.034 | 1.00 | 56.96 | C |
| ATOM | 3963 | CB | SER | A | 252 | 4.562 | 16.585 | 6.606 | 1.00 | 59.16 | C |
| ATOM | 3966 | OG | SER | A | 252 | 5.524 | 16.922 | 5.592 | 1.00 | 58.00 | O |
| ATOM | 3968 | C | SER | A | 252 | 2.413 | 17.837 | 6.419 | 1.00 | 56.47 | C |
| ATOM | 3969 | O | SER | A | 252 | 2.110 | 18.098 | 7.575 | 1.00 | 51.08 | O |
| ATOM | 3970 | N | SER | A | 253 | 2.089 | 18.610 | 5.401 | 1.00 | 59.91 | N |
| ATOM | 3972 | CA | SER | A | 253 | 1.690 | 20.003 | 5.568 | 1.00 | 59.18 | C |
| ATOM | 3974 | CB | SER | A | 253 | 1.581 | 20.643 | 4.173 | 1.00 | 57.63 | C |
| ATOM | 3977 | OG | SER | A | 253 | 1.316 | 22.026 | 4.257 | 1.00 | 55.74 | O |
| ATOM | 3979 | C | SER | A | 253 | 0.484 | 20.373 | 6.441 | 1.00 | 60.11 | C |
| ATOM | 3980 | O | SER | A | 253 | 0.075 | 21.526 | 6.428 | 1.00 | 59.14 | O |
| ATOM | 3981 | N | THR | A | 254 | −0.099 | 19.451 | 7.200 | 1.00 | 60.35 | N |
| ATOM | 3983 | CA | THR | A | 254 | −1.147 | 19.897 | 8.117 | 1.00 | 60.80 | C |
| ATOM | 3985 | CB | THR | A | 254 | −2.318 | 18.951 | 8.293 | 1.00 | 60.71 | C |
| ATOM | 3987 | OG1 | THR | A | 254 | −1.905 | 17.797 | 9.043 | 1.00 | 56.78 | O |
| ATOM | 3989 | CG2 | THR | A | 254 | −2.858 | 18.465 | 6.955 | 1.00 | 60.72 | C |
| ATOM | 3993 | C | THR | A | 254 | −0.473 | 20.059 | 9.435 | 1.00 | 64.58 | C |
| ATOM | 3994 | O | THR | A | 254 | −1.121 | 20.164 | 10.478 | 1.00 | 68.02 | O |
| ATOM | 3995 | N | GLU | A | 255 | 0.848 | 20.043 | 9.385 | 1.00 | 66.30 | N |
| ATOM | 3997 | CA | GLU | A | 255 | 1.626 | 20.400 | 10.537 | 1.00 | 69.56 | C |
| ATOM | 3999 | CB | GLU | A | 255 | 1.998 | 19.192 | 11.398 | 1.00 | 70.30 | C |
| ATOM | 4002 | CG | GLU | A | 255 | 0.804 | 18.717 | 12.245 | 1.00 | 70.88 | C |
| ATOM | 4005 | CD | GLU | A | 255 | 1.164 | 18.170 | 13.624 | 1.00 | 72.29 | C |
| ATOM | 4006 | OE1 | GLU | A | 255 | 2.307 | 18.397 | 14.115 | 1.00 | 79.50 | O |
| ATOM | 4007 | OE2 | GLU | A | 255 | 0.285 | 17.519 | 14.229 | 1.00 | 62.93 | O |
| ATOM | 4008 | C | GLU | A | 255 | 2.810 | 21.164 | 10.000 | 1.00 | 72.57 | C |
| ATOM | 4009 | O | GLU | A | 255 | 3.248 | 20.966 | 8.852 | 1.00 | 74.27 | O |
| ATOM | 4010 | N | LYS | A | 256 | 3.298 | 22.083 | 10.818 | 1.00 | 72.10 | N |
| ATOM | 4012 | CA | LYS | A | 256 | 4.408 | 22.901 | 10.411 | 1.00 | 68.65 | C |
| ATOM | 4014 | CB | LYS | A | 256 | 4.017 | 24.356 | 10.254 | 1.00 | 69.61 | C |
| ATOM | 4017 | CG | LYS | A | 256 | 3.138 | 24.580 | 9.037 | 1.00 | 76.54 | C |
| ATOM | 4020 | CD | LYS | A | 256 | 3.813 | 24.086 | 7.737 | 1.00 | 79.94 | C |
| ATOM | 4023 | CE | LYS | A | 256 | 3.261 | 24.791 | 6.471 | 1.00 | 79.27 | C |
| ATOM | 4026 | NZ | LYS | A | 256 | 2.321 | 23.945 | 5.676 | 1.00 | 79.72 | N |
| ATOM | 4030 | C | LYS | A | 256 | 5.429 | 22.704 | 11.462 | 1.00 | 64.96 | C |
| ATOM | 4031 | O | LYS | A | 256 | 5.097 | 22.569 | 12.658 | 1.00 | 51.53 | O |
| ATOM | 4032 | N | PHE | A | 257 | 6.664 | 22.674 | 10.972 | 1.00 | 61.73 | N |
| ATOM | 4034 | CA | PHE | A | 257 | 7.826 | 22.397 | 11.762 | 1.00 | 60.99 | C |
| ATOM | 4036 | CB | PHE | A | 257 | 8.370 | 21.026 | 11.354 | 1.00 | 65.88 | C |
| ATOM | 4039 | CG | PHE | A | 257 | 7.338 | 19.922 | 11.397 | 1.00 | 67.36 | C |
| ATOM | 4040 | CD1 | PHE | A | 257 | 6.875 | 19.347 | 10.232 | 1.00 | 71.73 | C |
| ATOM | 4042 | CE1 | PHE | A | 257 | 5.931 | 18.341 | 10.278 | 1.00 | 72.34 | C |
| ATOM | 4044 | CZ | PHE | A | 257 | 5.447 | 17.903 | 11.503 | 1.00 | 71.58 | C |
| ATOM | 4046 | CE2 | PHE | A | 257 | 5.910 | 18.476 | 12.667 | 1.00 | 68.20 | C |
| ATOM | 4048 | CD2 | PHE | A | 257 | 6.840 | 19.469 | 12.611 | 1.00 | 66.34 | C |
| ATOM | 4050 | C | PHE | A | 257 | 8.811 | 23.466 | 11.392 | 1.00 | 59.35 | C |
| ATOM | 4051 | O | PHE | A | 257 | 8.716 | 24.049 | 10.333 | 1.00 | 58.22 | O |
| ATOM | 4052 | N | PRO | A | 258 | 9.766 | 23.724 | 12.257 | 1.00 | 61.91 | N |
| ATOM | 4053 | CA | PRO | A | 258 | 10.792 | 24.731 | 11.983 | 1.00 | 65.38 | C |
| ATOM | 4055 | CB | PRO | A | 258 | 11.351 | 25.053 | 13.358 | 1.00 | 66.17 | C |
| ATOM | 4058 | CG | PRO | A | 258 | 10.803 | 23.979 | 14.309 | 1.00 | 64.85 | C |
| ATOM | 4061 | CD | PRO | A | 258 | 9.949 | 23.054 | 13.546 | 1.00 | 61.29 | C |
| ATOM | 4064 | C | PRO | A | 258 | 11.875 | 24.094 | 11.144 | 1.00 | 69.63 | C |
| ATOM | 4065 | O | PRO | A | 258 | 12.156 | 22.912 | 11.335 | 1.00 | 67.19 | O |
| ATOM | 4066 | N | ASP | A | 259 | 12.485 | 24.858 | 10.248 | 1.00 | 74.52 | N |
| ATOM | 4068 | CA | ASP | A | 259 | 13.450 | 24.293 | 9.308 | 1.00 | 76.21 | C |
| ATOM | 4070 | CB | ASP | A | 259 | 14.038 | 25.411 | 8.459 | 1.00 | 78.78 | C |
| ATOM | 4073 | CG | ASP | A | 259 | 12.952 | 26.362 | 7.934 | 1.00 | 85.56 | C |
| ATOM | 4074 | OD1 | ASP | A | 259 | 12.355 | 26.110 | 6.853 | 1.00 | 85.56 | O |
| ATOM | 4075 | OD2 | ASP | A | 259 | 12.604 | 27.384 | 8.566 | 1.00 | 94.62 | O |
| ATOM | 4076 | C | ASP | A | 259 | 14.514 | 23.433 | 10.009 | 1.00 | 75.34 | C |
| ATOM | 4077 | O | ASP | A | 259 | 14.811 | 22.315 | 9.560 | 1.00 | 77.54 | O |
| ATOM | 4078 | N | GLY | A | 260 | 15.056 | 23.927 | 11.119 | 1.00 | 68.42 | N |
| ATOM | 4080 | CA | GLY | A | 260 | 16.038 | 23.173 | 11.867 | 1.00 | 65.64 | C |
| ATOM | 4083 | C | GLY | A | 260 | 15.782 | 21.681 | 11.757 | 1.00 | 64.85 | C |
| ATOM | 4084 | O | GLY | A | 260 | 16.705 | 20.864 | 11.545 | 1.00 | 65.50 | O |
| ATOM | 4085 | N | PHE | A | 261 | 14.512 | 21.331 | 11.912 | 1.00 | 62.03 | N |
| ATOM | 4087 | CA | PHE | A | 261 | 14.039 | 19.953 | 11.778 | 1.00 | 60.14 | C |
| ATOM | 4089 | CB | PHE | A | 261 | 12.545 | 19.893 | 12.146 | 1.00 | 59.84 | C |
| ATOM | 4092 | CG | PHE | A | 261 | 11.901 | 18.542 | 11.976 | 1.00 | 53.88 | C |
| ATOM | 4093 | CD1 | PHE | A | 261 | 12.040 | 17.584 | 12.931 | 1.00 | 50.78 | C |
| ATOM | 4095 | CE1 | PHE | A | 261 | 11.450 | 16.369 | 12.790 | 1.00 | 51.53 | C |
| ATOM | 4097 | CZ | PHE | A | 261 | 10.699 | 16.096 | 11.676 | 1.00 | 55.50 | C |
| ATOM | 4099 | CE2 | PHE | A | 261 | 10.542 | 17.043 | 10.705 | 1.00 | 51.48 | C |
| ATOM | 4101 | CD2 | PHE | A | 261 | 11.140 | 18.260 | 10.859 | 1.00 | 55.00 | C |
| ATOM | 4103 | C | PHE | A | 261 | 14.271 | 19.432 | 10.364 | 1.00 | 60.30 | C |
| ATOM | 4104 | O | PHE | A | 261 | 14.921 | 18.396 | 10.165 | 1.00 | 62.84 | O |
| ATOM | 4105 | N | TRP | A | 262 | 13.778 | 20.132 | 9.363 | 1.00 | 52.97 | N |
| ATOM | 4107 | CA | TRP | A | 262 | 13.930 | 19.551 | 8.071 | 1.00 | 54.85 | C |
| ATOM | 4109 | CB | TRP | A | 262 | 13.154 | 20.339 | 7.017 | 1.00 | 56.23 | C |
| ATOM | 4112 | CG | TRP | A | 262 | 11.652 | 20.135 | 7.269 | 1.00 | 61.23 | C |
| ATOM | 4113 | CD1 | TRP | A | 262 | 10.780 | 21.050 | 7.761 | 1.00 | 66.49 | C |
| ATOM | 4115 | NE1 | TRP | A | 262 | 9.525 | 20.510 | 7.874 | 1.00 | 68.50 | N |
| ATOM | 4117 | CE2 | TRP | A | 262 | 9.565 | 19.213 | 7.447 | 1.00 | 67.88 | C |
| ATOM | 4118 | CD2 | TRP | A | 262 | 10.889 | 18.941 | 7.064 | 1.00 | 61.30 | C |
| ATOM | 4119 | CE3 | TRP | A | 262 | 11.188 | 17.669 | 6.586 | 1.00 | 64.17 | C |
| ATOM | 4121 | CZ3 | TRP | A | 262 | 10.177 | 16.728 | 6.510 | 1.00 | 63.85 | C |
| ATOM | 4123 | CH2 | TRP | A | 262 | 8.879 | 17.034 | 6.896 | 1.00 | 66.57 | C |
| ATOM | 4125 | CZ2 | TRP | A | 262 | 8.547 | 18.266 | 7.365 | 1.00 | 66.36 | C |
| ATOM | 4127 | C | TRP | A | 262 | 15.391 | 19.391 | 7.785 | 1.00 | 53.56 | C |
| ATOM | 4128 | O | TRP | A | 262 | 15.769 | 18.849 | 6.757 | 1.00 | 56.81 | O |
| ATOM | 4129 | N | LEU | A | 263 | 16.220 | 19.839 | 8.725 | 1.00 | 58.44 | N |
| ATOM | 4131 | CA | LEU | A | 263 | 17.677 | 19.898 | 8.531 | 1.00 | 58.98 | C |
| ATOM | 4133 | CB | LEU | A | 263 | 18.170 | 21.276 | 8.866 | 1.00 | 57.08 | C |
| ATOM | 4136 | CG | LEU | A | 263 | 18.031 | 22.222 | 7.709 | 1.00 | 59.71 | C |
| ATOM | 4138 | CD1 | LEU | A | 263 | 18.483 | 23.577 | 8.206 | 1.00 | 59.96 | C |
| ATOM | 4142 | CD2 | LEU | A | 263 | 18.875 | 21.732 | 6.527 | 1.00 | 60.81 | C |
| ATOM | 4146 | C | LEU | A | 263 | 18.468 | 18.983 | 9.398 | 1.00 | 62.54 | C |
| ATOM | 4147 | O | LEU | A | 263 | 19.686 | 19.128 | 9.526 | 1.00 | 61.58 | O |
| ATOM | 4148 | N | GLY | A | 264 | 17.778 | 18.046 | 10.012 | 1.00 | 67.37 | N |
| ATOM | 4150 | CA | GLY | A | 264 | 18.436 | 17.118 | 10.893 | 1.00 | 70.18 | C |
| ATOM | 4153 | C | GLY | A | 264 | 18.788 | 17.817 | 12.179 | 1.00 | 72.26 | C |
| ATOM | 4154 | O | GLY | A | 264 | 18.910 | 17.143 | 13.194 | 1.00 | 76.04 | O |
| ATOM | 4155 | N | GLU | A | 265 | 18.927 | 19.149 | 12.149 | 1.00 | 71.01 | N |
| ATOM | 4157 | CA | GLU | A | 265 | 19.437 | 19.880 | 13.311 | 1.00 | 71.40 | C |
| ATOM | 4159 | CB | GLU | A | 265 | 19.648 | 21.383 | 13.034 | 1.00 | 71.95 | C |
| ATOM | 4162 | CG | GLU | A | 265 | 20.982 | 21.698 | 12.353 | 1.00 | 74.37 | C |
| ATOM | 4165 | CD | GLU | A | 265 | 20.958 | 22.955 | 11.471 | 1.00 | 74.89 | C |
| ATOM | 4166 | OE1 | GLU | A | 265 | 21.757 | 23.022 | 10.500 | 1.00 | 63.91 | O |
| ATOM | 4167 | OE2 | GLU | A | 265 | 20.142 | 23.876 | 11.745 | 1.00 | 80.45 | O |
| ATOM | 4168 | C | GLU | A | 265 | 18.592 | 19.716 | 14.547 | 1.00 | 70.60 | C |
| ATOM | 4169 | O | GLU | A | 265 | 19.099 | 19.216 | 15.542 | 1.00 | 72.28 | O |
| ATOM | 4170 | N | GLN | A | 266 | 17.322 | 20.118 | 14.516 | 1.00 | 68.36 | N |
| ATOM | 4172 | CA | GLN | A | 266 | 16.574 | 20.094 | 15.769 | 1.00 | 68.13 | C |
| ATOM | 4174 | CB | GLN | A | 266 | 16.036 | 21.431 | 16.254 | 1.00 | 71.81 | C |
| ATOM | 4177 | CG | GLN | A | 266 | 15.288 | 22.275 | 15.282 | 1.00 | 73.46 | C |
| ATOM | 4180 | CD | GLN | A | 266 | 15.211 | 23.702 | 15.831 | 1.00 | 80.14 | C |
| ATOM | 4181 | OE1 | GLN | A | 266 | 16.098 | 24.530 | 15.578 | 1.00 | 78.16 | O |
| ATOM | 4182 | NE2 | GLN | A | 266 | 14.163 | 23.982 | 16.604 | 1.00 | 81.99 | N |
| ATOM | 4185 | C | GLN | A | 266 | 15.495 | 19.132 | 15.770 | 1.00 | 65.19 | C |
| ATOM | 4186 | O | GLN | A | 266 | 14.952 | 18.739 | 14.761 | 1.00 | 68.32 | O |
| ATOM | 4187 | N | LEU | A | 267 | 15.179 | 18.751 | 16.973 | 1.00 | 62.13 | N |
| ATOM | 4189 | CA | LEU | A | 267 | 14.400 | 17.604 | 17.135 | 1.00 | 60.81 | C |
| ATOM | 4191 | CB | LEU | A | 267 | 14.990 | 16.849 | 18.335 | 1.00 | 60.23 | C |
| ATOM | 4194 | CG | LEU | A | 267 | 14.662 | 17.271 | 19.724 | 1.00 | 56.50 | C |
| ATOM | 4196 | CD1 | LEU | A | 267 | 13.130 | 17.340 | 19.756 | 1.00 | 54.80 | C |
| ATOM | 4200 | CD2 | LEU | A | 267 | 15.234 | 16.210 | 20.656 | 1.00 | 52.66 | C |
| ATOM | 4204 | C | LEU | A | 267 | 12.988 | 18.098 | 17.205 | 1.00 | 60.54 | C |
| ATOM | 4205 | O | LEU | A | 267 | 12.771 | 19.279 | 17.110 | 1.00 | 67.47 | O |
| ATOM | 4206 | N | VAL | A | 268 | 12.013 | 17.214 | 17.315 | 1.00 | 67.35 | N |
| ATOM | 4208 | CA | VAL | A | 268 | 10.617 | 17.635 | 17.398 | 1.00 | 64.13 | C |
| ATOM | 4210 | CB | VAL | A | 268 | 9.915 | 17.517 | 16.071 | 1.00 | 64.12 | C |
| ATOM | 4212 | CG1 | VAL | A | 268 | 8.431 | 17.553 | 16.280 | 1.00 | 64.85 | C |
| ATOM | 4216 | CG2 | VAL | A | 268 | 10.332 | 18.629 | 15.148 | 1.00 | 68.52 | C |
| ATOM | 4220 | C | VAL | A | 268 | 9.895 | 16.719 | 18.348 | 1.00 | 65.02 | C |
| ATOM | 4221 | O | VAL | A | 268 | 10.339 | 15.595 | 18.595 | 1.00 | 67.72 | O |
| ATOM | 4222 | N | CYS | A | 269 | 8.774 | 17.185 | 18.876 | 1.00 | 64.09 | N |
| ATOM | 4224 | CA | CYS | A | 269 | 8.045 | 16.373 | 19.822 | 1.00 | 64.62 | C |
| ATOM | 4226 | CB | CYS | A | 269 | 8.506 | 16.794 | 21.203 | 1.00 | 66.31 | C |
| ATOM | 4229 | SG | CYS | A | 269 | 10.286 | 17.212 | 21.240 | 1.00 | 69.02 | S |
| ATOM | 4230 | C | CYS | A | 269 | 6.523 | 16.452 | 19.735 | 1.00 | 64.55 | C |
| ATOM | 4231 | O | CYS | A | 269 | 5.943 | 17.350 | 19.089 | 1.00 | 67.95 | O |
| ATOM | 4232 | N | TRP | A | 270 | 5.899 | 15.479 | 20.395 | 1.00 | 61.04 | N |
| ATOM | 4234 | CA | TRP | A | 270 | 4.444 | 15.334 | 20.512 | 1.00 | 61.84 | C |
| ATOM | 4236 | CB | TRP | A | 270 | 3.764 | 14.554 | 19.371 | 1.00 | 56.93 | C |
| ATOM | 4239 | CG | TRP | A | 270 | 3.867 | 15.143 | 17.967 | 1.00 | 58.64 | C |
| ATOM | 4240 | CD1 | TRP | A | 270 | 3.041 | 16.067 | 17.388 | 1.00 | 59.48 | C |
| ATOM | 4242 | NE1 | TRP | A | 270 | 3.452 | 16.344 | 16.105 | 1.00 | 54.79 | N |
| ATOM | 4244 | CE2 | TRP | A | 270 | 4.562 | 15.588 | 15.825 | 1.00 | 57.15 | C |
| ATOM | 4245 | CD2 | TRP | A | 270 | 4.848 | 14.816 | 16.971 | 1.00 | 56.20 | C |
| ATOM | 4246 | CE3 | TRP | A | 270 | 5.948 | 13.956 | 16.939 | 1.00 | 49.93 | C |
| ATOM | 4248 | CZ3 | TRP | A | 270 | 6.708 | 13.889 | 15.796 | 1.00 | 55.27 | C |
| ATOM | 4250 | CH2 | TRP | A | 270 | 6.395 | 14.667 | 14.667 | 1.00 | 58.79 | C |
| ATOM | 4252 | CZ2 | TRP | A | 270 | 5.331 | 15.517 | 14.662 | 1.00 | 55.58 | C |
| ATOM | 4254 | C | TRP | A | 270 | 4.288 | 14.549 | 21.813 | 1.00 | 64.90 | C |
| ATOM | 4255 | O | TRP | A | 270 | 5.212 | 13.830 | 22.231 | 1.00 | 64.95 | O |
| ATOM | 4256 | N | GLN | A | 271 | 3.112 | 14.664 | 22.427 | 1.00 | 64.50 | N |
| ATOM | 4258 | CA | GLN | A | 271 | 2.891 | 14.202 | 23.791 | 1.00 | 59.95 | C |
| ATOM | 4260 | CB | GLN | A | 271 | 1.547 | 14.688 | 24.328 | 1.00 | 58.83 | C |
| ATOM | 4263 | CG | GLN | A | 271 | 1.117 | 16.045 | 23.773 | 1.00 | 65.33 | C |
| ATOM | 4266 | CD | GLN | A | 271 | 1.086 | 16.093 | 22.229 | 1.00 | 63.95 | C |
| ATOM | 4267 | OE1 | GLN | A | 271 | 0.909 | 15.057 | 21.559 | 1.00 | 70.15 | O |
| ATOM | 4268 | NE2 | GLN | A | 271 | 1.256 | 17.287 | 21.674 | 1.00 | 54.26 | N |
| ATOM | 4271 | C | GLN | A | 271 | 2.931 | 12.731 | 23.697 | 1.00 | 56.78 | C |
| ATOM | 4272 | O | GLN | A | 271 | 2.634 | 12.186 | 22.647 | 1.00 | 57.25 | O |
| ATOM | 4273 | N | ALA | A | 272 | 3.274 | 12.068 | 24.774 | 1.00 | 55.14 | N |
| ATOM | 4275 | CA | ALA | A | 272 | 3.457 | 10.637 | 24.671 | 1.00 | 58.98 | C |
| ATOM | 4277 | CB | ALA | A | 272 | 3.430 | 9.985 | 26.025 | 1.00 | 59.97 | C |
| ATOM | 4281 | C | ALA | A | 272 | 2.422 | 10.009 | 23.754 | 1.00 | 59.71 | C |
| ATOM | 4282 | O | ALA | A | 272 | 1.365 | 10.587 | 23.482 | 1.00 | 57.10 | O |
| ATOM | 4283 | N | GLY | A | 273 | 2.782 | 8.821 | 23.271 | 1.00 | 62.02 | N |
| ATOM | 4285 | CA | GLY | A | 273 | 1.952 | 7.965 | 22.431 | 1.00 | 59.67 | C |
| ATOM | 4288 | C | GLY | A | 273 | 0.981 | 8.622 | 21.490 | 1.00 | 58.98 | C |
| ATOM | 4289 | O | GLY | A | 273 | 0.009 | 7.967 | 21.115 | 1.00 | 61.66 | O |
| ATOM | 4290 | N | THR | A | 274 | 1.243 | 9.878 | 21.113 | 1.00 | 57.29 | N |
| ATOM | 4292 | CA | THR | A | 274 | 0.351 | 10.661 | 20.258 | 1.00 | 57.22 | C |
| ATOM | 4294 | CB | THR | A | 274 | −0.108 | 11.911 | 21.023 | 1.00 | 63.52 | C |
| ATOM | 4296 | OG1 | THR | A | 274 | 1.035 | 12.634 | 21.513 | 1.00 | 63.82 | O |
| ATOM | 4298 | CG2 | THR | A | 274 | −0.897 | 11.537 | 22.291 | 1.00 | 65.12 | C |
| ATOM | 4302 | C | THR | A | 274 | 0.994 | 11.128 | 18.943 | 1.00 | 54.80 | C |
| ATOM | 4303 | O | THR | A | 274 | 0.493 | 12.032 | 18.256 | 1.00 | 49.79 | O |
| ATOM | 4304 | N | THR | A | 275 | 2.104 | 10.534 | 18.560 | 1.00 | 50.99 | N |
| ATOM | 4306 | CA | THR | A | 275 | 2.712 | 11.010 | 17.345 | 1.00 | 50.86 | C |
| ATOM | 4308 | CB | THR | A | 275 | 4.033 | 10.303 | 17.107 | 1.00 | 53.40 | C |
| ATOM | 4310 | OG1 | THR | A | 275 | 4.793 | 10.248 | 18.331 | 1.00 | 53.69 | O |
| ATOM | 4312 | CG2 | THR | A | 275 | 4.901 | 11.074 | 16.111 | 1.00 | 48.79 | C |
| ATOM | 4316 | C | THR | A | 275 | 1.736 | 10.679 | 16.226 | 1.00 | 52.73 | C |
| ATOM | 4317 | O | THR | A | 275 | 1.230 | 9.560 | 16.129 | 1.00 | 55.77 | O |
| ATOM | 4318 | N | PRO | A | 276 | 1.456 | 11.656 | 15.399 | 1.00 | 48.39 | N |
| ATOM | 4319 | CA | PRO | A | 276 | 0.602 | 11.493 | 14.226 | 1.00 | 50.13 | C |
| ATOM | 4321 | CB | PRO | A | 276 | 0.223 | 12.950 | 13.952 | 1.00 | 50.02 | C |
| ATOM | 4324 | CG | PRO | A | 276 | 1.513 | 13.620 | 14.213 | 1.00 | 50.60 | C |
| ATOM | 4327 | CD | PRO | A | 276 | 1.877 | 13.055 | 15.554 | 1.00 | 53.12 | C |
| ATOM | 4330 | C | PRO | A | 276 | 1.265 | 10.862 | 12.974 | 1.00 | 42.85 | C |
| ATOM | 4331 | O | PRO | A | 276 | 1.501 | 11.537 | 11.980 | 1.00 | 39.08 | O |
| ATOM | 4332 | N | TRP | A | 277 | 1.527 | 9.567 | 12.997 | 1.00 | 46.18 | N |
| ATOM | 4334 | CA | TRP | A | 277 | 2.246 | 8.919 | 11.879 | 1.00 | 46.30 | C |
| ATOM | 4336 | CB | TRP | A | 277 | 2.339 | 7.449 | 12.146 | 1.00 | 41.35 | C |
| ATOM | 4339 | CG | TRP | A | 277 | 3.154 | 7.098 | 13.355 | 1.00 | 41.81 | C |
| ATOM | 4340 | CD1 | TRP | A | 277 | 2.696 | 6.545 | 14.489 | 1.00 | 45.45 | C |
| ATOM | 4342 | NE1 | TRP | A | 277 | 3.726 | 6.346 | 15.376 | 1.00 | 46.12 | N |
| ATOM | 4344 | CE2 | TRP | A | 277 | 4.886 | 6.784 | 14.809 | 1.00 | 40.67 | C |
| ATOM | 4345 | CD2 | TRP | A | 277 | 4.565 | 7.263 | 13.538 | 1.00 | 42.28 | C |
| ATOM | 4346 | CE3 | TRP | A | 277 | 5.596 | 7.775 | 12.752 | 1.00 | 45.83 | C |
| ATOM | 4348 | CZ3 | TRP | A | 277 | 6.872 | 7.785 | 13.257 | 1.00 | 41.25 | C |
| ATOM | 4350 | CH2 | TRP | A | 277 | 7.153 | 7.296 | 14.526 | 1.00 | 37.21 | C |
| ATOM | 4352 | CZ2 | TRP | A | 277 | 6.185 | 6.798 | 15.316 | 1.00 | 40.09 | C |
| ATOM | 4354 | C | TRP | A | 277 | 1.603 | 9.129 | 10.492 | 1.00 | 51.02 | C |
| ATOM | 4355 | O | TRP | A | 277 | 2.252 | 9.510 | 9.497 | 1.00 | 55.36 | O |
| ATOM | 4356 | N | ASN | A | 278 | 0.311 | 8.883 | 10.450 | 1.00 | 51.19 | N |
| ATOM | 4358 | CA | ASN | A | 278 | −0.467 | 9.067 | 9.265 | 1.00 | 46.14 | C |
| ATOM | 4360 | CB | ASN | A | 278 | −1.925 | 8.947 | 9.660 | 1.00 | 51.01 | C |
| ATOM | 4363 | CG | ASN | A | 278 | −2.446 | 10.207 | 10.318 | 1.00 | 53.16 | C |
| ATOM | 4364 | OD1 | ASN | A | 278 | −2.406 | 11.277 | 9.728 | 1.00 | 56.07 | O |
| ATOM | 4365 | ND2 | ASN | A | 278 | −2.929 | 10.084 | 11.540 | 1.00 | 43.67 | N |
| ATOM | 4368 | C | ASN | A | 278 | −0.250 | 10.367 | 8.505 | 1.00 | 47.14 | C |
| ATOM | 4369 | O | ASN | A | 278 | −0.443 | 10.409 | 7.281 | 1.00 | 55.65 | O |
| ATOM | 4370 | N | ILE | A | 279 | 0.154 | 11.459 | 9.136 | 1.00 | 45.86 | N |
| ATOM | 4372 | CA | ILE | A | 279 | 0.262 | 12.647 | 8.283 | 1.00 | 40.58 | C |
| ATOM | 4374 | CB | ILE | A | 279 | 0.201 | 13.912 | 9.019 | 1.00 | 41.95 | C |
| ATOM | 4376 | CG1 | ILE | A | 279 | 1.508 | 14.087 | 9.747 | 1.00 | 44.28 | C |
| ATOM | 4379 | CD1 | ILE | A | 279 | 1.810 | 15.503 | 10.041 | 1.00 | 48.63 | C |
| ATOM | 4383 | CG2 | ILE | A | 279 | −1.078 | 13.936 | 9.940 | 1.00 | 40.98 | C |
| ATOM | 4387 | C | ILE | A | 279 | 1.502 | 12.647 | 7.511 | 1.00 | 41.25 | C |
| ATOM | 4388 | O | ILE | A | 279 | 1.571 | 13.405 | 6.527 | 1.00 | 41.55 | O |
| ATOM | 4389 | N | PHE | A | 280 | 2.494 | 11.830 | 7.932 | 1.00 | 42.95 | N |
| ATOM | 4391 | CA | PHE | A | 280 | 3.734 | 11.696 | 7.155 | 1.00 | 43.36 | C |
| ATOM | 4393 | CB | PHE | A | 280 | 4.914 | 11.371 | 8.031 | 1.00 | 47.33 | C |
| ATOM | 4396 | CG | PHE | A | 280 | 5.285 | 12.438 | 9.006 | 1.00 | 48.46 | C |
| ATOM | 4397 | CD1 | PHE | A | 280 | 6.155 | 13.457 | 8.641 | 1.00 | 52.35 | C |
| ATOM | 4399 | CE1 | PHE | A | 280 | 6.512 | 14.437 | 9.555 | 1.00 | 55.06 | C |
| ATOM | 4401 | CZ | PHE | A | 280 | 5.997 | 14.385 | 10.859 | 1.00 | 53.18 | C |
| ATOM | 4403 | CE2 | PHE | A | 280 | 5.131 | 13.352 | 11.213 | 1.00 | 48.87 | C |
| ATOM | 4405 | CD2 | PHE | A | 280 | 4.791 | 12.401 | 10.296 | 1.00 | 47.29 | C |
| ATOM | 4407 | C | PHE | A | 280 | 3.596 | 10.572 | 6.116 | 1.00 | 43.82 | C |
| ATOM | 4408 | O | PHE | A | 280 | 2.937 | 9.572 | 6.344 | 1.00 | 46.62 | O |
| ATOM | 4409 | N | PRO | A | 281 | 4.236 | 10.726 | 4.978 | 1.00 | 41.27 | N |
| ATOM | 4410 | CA | PRO | A | 281 | 4.127 | 9.768 | 3.874 | 1.00 | 35.61 | C |
| ATOM | 4412 | CB | PRO | A | 281 | 4.376 | 10.655 | 2.660 | 1.00 | 36.29 | C |
| ATOM | 4415 | CG | PRO | A | 281 | 5.171 | 11.767 | 3.144 | 1.00 | 35.91 | C |
| ATOM | 4418 | CD | PRO | A | 281 | 5.116 | 11.847 | 4.657 | 1.00 | 37.67 | C |
| ATOM | 4421 | C | PRO | A | 281 | 5.217 | 8.679 | 3.868 | 1.00 | 39.20 | C |
| ATOM | 4422 | O | PRO | A | 281 | 6.162 | 8.792 | 4.650 | 1.00 | 35.73 | O |
| ATOM | 4423 | N | VAL | A | 282 | 5.072 | 7.679 | 2.981 | 1.00 | 36.56 | N |
| ATOM | 4425 | CA | VAL | A | 282 | 5.987 | 6.562 | 2.819 | 1.00 | 35.89 | C |
| ATOM | 4427 | CB | VAL | A | 282 | 5.215 | 5.282 | 2.365 | 1.00 | 39.74 | C |
| ATOM | 4429 | CG1 | VAL | A | 282 | 4.205 | 4.818 | 3.377 | 1.00 | 36.87 | C |
| ATOM | 4433 | CG2 | VAL | A | 282 | 4.500 | 5.499 | 1.028 | 1.00 | 45.22 | C |
| ATOM | 4437 | C | VAL | A | 282 | 7.030 | 6.872 | 1.714 | 1.00 | 40.69 | C |
| ATOM | 4438 | O | VAL | A | 282 | 6.797 | 7.738 | 0.873 | 1.00 | 36.67 | O |
| ATOM | 4439 | N | ILE | A | 283 | 8.156 | 6.136 | 1.705 | 1.00 | 37.84 | N |
| ATOM | 4441 | CA | ILE | A | 283 | 9.241 | 6.356 | 0.712 | 1.00 | 38.96 | C |
| ATOM | 4443 | CB | ILE | A | 283 | 10.411 | 7.053 | 1.374 | 1.00 | 38.64 | C |
| ATOM | 4445 | CG1 | ILE | A | 283 | 9.963 | 8.318 | 2.070 | 1.00 | 44.14 | C |
| ATOM | 4448 | CD1 | ILE | A | 283 | 11.109 | 9.046 | 2.658 | 1.00 | 48.84 | C |
| ATOM | 4452 | CG2 | ILE | A | 283 | 11.481 | 7.314 | 0.311 | 1.00 | 29.11 | C |
| ATOM | 4456 | C | ILE | A | 283 | 9.899 | 5.255 | −0.053 | 1.00 | 41.40 | C |
| ATOM | 4457 | O | ILE | A | 283 | 10.851 | 4.668 | 0.441 | 1.00 | 62.61 | O |
| ATOM | 4458 | N | SER | A | 284 | 9.483 | 5.062 | −1.293 | 1.00 | 44.38 | N |
| ATOM | 4460 | CA | SER | A | 284 | 9.856 | 3.861 | −2.002 | 1.00 | 41.93 | C |
| ATOM | 4462 | CB | SER | A | 284 | 8.683 | 3.407 | −2.819 | 1.00 | 42.78 | C |
| ATOM | 4465 | OG | SER | A | 284 | 7.555 | 3.197 | −1.991 | 1.00 | 44.75 | O |
| ATOM | 4467 | C | SER | A | 284 | 11.040 | 4.075 | −2.897 | 1.00 | 44.34 | C |
| ATOM | 4468 | O | SER | A | 284 | 11.022 | 4.922 | −3.788 | 1.00 | 46.55 | O |
| ATOM | 4469 | N | LEU | A | 285 | 12.090 | 3.315 | −2.656 | 1.00 | 41.54 | N |
| ATOM | 4471 | CA | LEU | A | 285 | 13.223 | 3.384 | −3.521 | 1.00 | 37.02 | C |
| ATOM | 4473 | CB | LEU | A | 285 | 14.491 | 3.276 | −2.717 | 1.00 | 35.95 | C |
| ATOM | 4476 | CG | LEU | A | 285 | 14.805 | 4.402 | −1.704 | 1.00 | 38.01 | C |
| ATOM | 4478 | CD1 | LEU | A | 285 | 16.196 | 4.144 | −1.122 | 1.00 | 33.26 | C |
| ATOM | 4482 | CD2 | LEU | A | 285 | 14.762 | 5.845 | −2.251 | 1.00 | 33.22 | C |
| ATOM | 4486 | C | LEU | A | 285 | 13.043 | 2.195 | −4.451 | 1.00 | 38.43 | C |
| ATOM | 4487 | O | LEU | A | 285 | 12.880 | 1.086 | −3.978 | 1.00 | 43.06 | O |
| ATOM | 4488 | N | TYR | A | 286 | 13.017 | 2.399 | −5.759 | 1.00 | 32.94 | N |
| ATOM | 4490 | CA | TYR | A | 286 | 12.855 | 1.262 | −6.641 | 1.00 | 39.45 | C |
| ATOM | 4492 | CB | TYR | A | 286 | 12.115 | 1.611 | −7.910 | 1.00 | 40.17 | C |
| ATOM | 4495 | CG | TYR | A | 286 | 10.614 | 1.743 | −7.827 | 1.00 | 37.69 | C |
| ATOM | 4496 | CD1 | TYR | A | 286 | 10.025 | 2.618 | −6.958 | 1.00 | 35.61 | C |
| ATOM | 4498 | CE1 | TYR | A | 286 | 8.690 | 2.751 | −6.894 | 1.00 | 39.01 | C |
| ATOM | 4500 | CZ | TYR | A | 286 | 7.894 | 1.996 | −7.713 | 1.00 | 40.22 | C |
| ATOM | 4501 | OH | TYR | A | 286 | 6.518 | 2.137 | −7.632 | 1.00 | 32.05 | O |
| ATOM | 4503 | CE2 | TYR | A | 286 | 8.476 | 1.130 | −8.584 | 1.00 | 39.77 | C |
| ATOM | 4505 | CD2 | TYR | A | 286 | 9.821 | 1.023 | −8.648 | 1.00 | 35.63 | C |
| ATOM | 4507 | C | TYR | A | 286 | 14.207 | 0.832 | −7.119 | 1.00 | 36.96 | C |
| ATOM | 4508 | O | TYR | A | 286 | 14.990 | 1.654 | −7.483 | 1.00 | 41.56 | O |
| ATOM | 4509 | N | LEU | A | 287 | 14.465 | −0.461 | −7.177 | 1.00 | 37.56 | N |
| ATOM | 4511 | CA | LEU | A | 287 | 15.786 | −0.941 | −7.545 | 1.00 | 36.13 | C |
| ATOM | 4513 | CB | LEU | A | 287 | 16.385 | −1.695 | −6.391 | 1.00 | 37.50 | C |
| ATOM | 4516 | CG | LEU | A | 287 | 16.513 | −1.001 | −5.061 | 1.00 | 40.67 | C |
| ATOM | 4518 | CD1 | LEU | A | 287 | 16.957 | −2.079 | −4.008 | 1.00 | 42.47 | C |
| ATOM | 4522 | CD2 | LEU | A | 287 | 17.507 | 0.090 | −5.144 | 1.00 | 37.99 | C |
| ATOM | 4526 | C | LEU | A | 287 | 15.760 | −1.869 | −8.700 | 1.00 | 32.81 | C |
| ATOM | 4527 | O | LEU | A | 287 | 14.789 | −2.561 | −8.889 | 1.00 | 42.24 | O |
| ATOM | 4528 | N | MET | A | 288 | 16.849 | −1.897 | −9.447 | 1.00 | 37.48 | N |
| ATOM | 4530 | CA | MET | A | 288 | 17.040 | −2.777 | −10.632 | 1.00 | 43.22 | C |
| ATOM | 4532 | CB | MET | A | 288 | 18.349 | −2.356 | −11.349 | 1.00 | 49.18 | C |
| ATOM | 4535 | CG | MET | A | 288 | 19.046 | −3.419 | −12.208 | 1.00 | 53.21 | C |
| ATOM | 4538 | SD | MET | A | 288 | 20.373 | −2.706 | −13.301 | 1.00 | 68.95 | S |
| ATOM | 4539 | CE | MET | A | 288 | 19.993 | −0.904 | −13.311 | 1.00 | 59.14 | C |
| ATOM | 4543 | C | MET | A | 288 | 17.104 | −4.269 | −10.276 | 1.00 | 42.94 | C |
| ATOM | 4544 | O | MET | A | 288 | 17.771 | −4.671 | −9.328 | 1.00 | 46.50 | O |
| ATOM | 4545 | N | GLY | A | 289 | 16.446 | −5.098 | −11.055 | 1.00 | 40.57 | N |
| ATOM | 4547 | CA | GLY | A | 289 | 16.290 | −6.485 | −10.682 | 1.00 | 43.24 | C |
| ATOM | 4550 | C | GLY | A | 289 | 17.409 | −7.294 | −11.225 | 1.00 | 48.62 | C |
| ATOM | 4551 | O | GLY | A | 289 | 18.320 | −6.726 | −11.761 | 1.00 | 57.55 | O |
| ATOM | 4552 | N | GLU | A | 290 | 17.366 | −8.607 | −11.061 | 1.00 | 54.59 | N |
| ATOM | 4554 | CA | GLU | A | 290 | 18.352 | −9.464 | −11.692 | 1.00 | 60.08 | C |
| ATOM | 4556 | CB | GLU | A | 290 | 18.531 | −10.762 | −10.927 | 1.00 | 61.43 | C |
| ATOM | 4559 | CG | GLU | A | 290 | 19.091 | −10.548 | −9.538 | 1.00 | 61.36 | C |
| ATOM | 4562 | CD | GLU | A | 290 | 19.931 | −11.718 | −9.077 | 1.00 | 61.64 | C |
| ATOM | 4563 | OE1 | GLU | A | 290 | 19.365 | −12.576 | −8.331 | 1.00 | 47.44 | O |
| ATOM | 4564 | OE2 | GLU | A | 290 | 21.142 | −11.741 | −9.471 | 1.00 | 55.78 | O |
| ATOM | 4565 | C | GLU | A | 290 | 17.928 | −9.808 | −13.091 | 1.00 | 63.07 | C |
| ATOM | 4566 | O | GLU | A | 290 | 18.755 | −10.232 | −13.887 | 1.00 | 67.93 | O |
| ATOM | 4567 | N | VAL | A | 291 | 16.643 | −9.655 | −13.398 | 1.00 | 65.43 | N |
| ATOM | 4569 | CA | VAL | A | 291 | 16.189 | −9.910 | −14.769 | 1.00 | 66.54 | C |
| ATOM | 4571 | CB | VAL | A | 291 | 14.926 | −10.790 | −14.824 | 1.00 | 64.54 | C |
| ATOM | 4573 | CG1 | VAL | A | 291 | 14.666 | −11.196 | −16.218 | 1.00 | 66.22 | C |
| ATOM | 4577 | CG2 | VAL | A | 291 | 15.116 | −12.044 | −14.013 | 1.00 | 66.62 | C |
| ATOM | 4581 | C | VAL | A | 291 | 16.027 | −8.615 | −15.607 | 1.00 | 64.98 | C |
| ATOM | 4582 | O | VAL | A | 291 | 15.564 | −7.590 | −15.135 | 1.00 | 67.86 | O |
| ATOM | 4583 | N | THR | A | 292 | 16.432 | −8.687 | −16.862 | 1.00 | 64.83 | N |
| ATOM | 4585 | CA | THR | A | 292 | 16.387 | −7.554 | −17.776 | 1.00 | 67.55 | C |
| ATOM | 4587 | CB | THR | A | 292 | 16.783 | −8.068 | −19.139 | 1.00 | 69.56 | C |
| ATOM | 4589 | OG1 | THR | A | 292 | 17.890 | −8.965 | −18.992 | 1.00 | 74.68 | O |
| ATOM | 4591 | CG2 | THR | A | 292 | 17.325 | −6.980 | −19.982 | 1.00 | 70.93 | C |
| ATOM | 4595 | C | THR | A | 292 | 15.015 | −6.878 | −17.870 | 1.00 | 67.32 | C |
| ATOM | 4596 | O | THR | A | 292 | 14.002 | −7.532 | −18.098 | 1.00 | 64.26 | O |
| ATOM | 4597 | N | GLN | A | 293 | 14.989 | −5.562 | −17.705 | 1.00 | 65.20 | N |
| ATOM | 4599 | CA | GLN | A | 293 | 13.735 | −4.852 | −17.700 | 1.00 | 65.67 | C |
| ATOM | 4601 | CB | GLN | A | 293 | 12.749 | −5.543 | −18.648 | 1.00 | 68.34 | C |
| ATOM | 4604 | CG | GLN | A | 293 | 12.971 | −5.013 | −19.979 | 1.00 | 69.90 | C |
| ATOM | 4607 | CD | GLN | A | 293 | 13.563 | −3.662 | −19.780 | 1.00 | 73.82 | C |
| ATOM | 4608 | OE1 | GLN | A | 293 | 14.704 | −3.406 | −20.178 | 1.00 | 76.41 | O |
| ATOM | 4609 | NE2 | GLN | A | 293 | 12.804 | −2.784 | −19.105 | 1.00 | 70.84 | N |
| ATOM | 4612 | C | GLN | A | 293 | 13.057 | −4.736 | −16.364 | 1.00 | 63.03 | C |
| ATOM | 4613 | O | GLN | A | 293 | 12.284 | −3.803 | −16.127 | 1.00 | 67.21 | O |
| ATOM | 4614 | N | GLN | A | 294 | 13.367 | −5.661 | −15.484 | 1.00 | 57.71 | N |
| ATOM | 4616 | CA | GLN | A | 294 | 12.562 | −5.859 | −14.307 | 1.00 | 56.14 | C |
| ATOM | 4618 | CB | GLN | A | 294 | 12.735 | −7.332 | −13.870 | 1.00 | 56.19 | C |
| ATOM | 4621 | CG | GLN | A | 294 | 11.579 | −7.951 | −13.090 | 1.00 | 56.15 | C |
| ATOM | 4624 | CD | GLN | A | 294 | 11.247 | −9.407 | −13.475 | 1.00 | 55.02 | C |
| ATOM | 4625 | OE1 | GLN | A | 294 | 10.962 | −10.255 | −12.596 | 1.00 | 46.01 | O |
| ATOM | 4626 | NE2 | GLN | A | 294 | 11.271 | −9.693 | −14.777 | 1.00 | 49.71 | N |
| ATOM | 4629 | C | GLN | A | 294 | 13.045 | −4.905 | −13.257 | 1.00 | 51.59 | C |
| ATOM | 4630 | O | GLN | A | 294 | 14.132 | −4.397 | −13.369 | 1.00 | 50.97 | O |
| ATOM | 4631 | N | SER | A | 295 | 12.229 | −4.625 | −12.260 | 1.00 | 47.06 | N |
| ATOM | 4633 | CA | SER | A | 295 | 12.740 | −3.950 | −11.063 | 1.00 | 48.50 | C |
| ATOM | 4635 | CB | SER | A | 295 | 12.882 | −2.483 | −11.321 | 1.00 | 42.42 | C |
| ATOM | 4638 | OG | SER | A | 295 | 11.655 | −2.067 | −11.834 | 1.00 | 48.16 | O |
| ATOM | 4640 | C | SER | A | 295 | 11.773 | −4.171 | −9.895 | 1.00 | 46.17 | C |
| ATOM | 4641 | O | SER | A | 295 | 10.676 | −4.726 | −10.059 | 1.00 | 48.16 | O |
| ATOM | 4642 | N | PHE | A | 296 | 12.146 | −3.729 | −8.721 | 1.00 | 37.72 | N |
| ATOM | 4644 | CA | PHE | A | 296 | 11.236 | −3.922 | −7.615 | 1.00 | 39.89 | C |
| ATOM | 4646 | CB | PHE | A | 296 | 11.533 | −5.236 | −6.978 | 1.00 | 39.36 | C |
| ATOM | 4649 | CG | PHE | A | 296 | 12.886 | −5.286 | −6.295 | 1.00 | 32.98 | C |
| ATOM | 4650 | CD1 | PHE | A | 296 | 13.035 | −4.863 | −5.018 | 1.00 | 37.10 | C |
| ATOM | 4652 | CE1 | PHE | A | 296 | 14.225 | −4.924 | −4.406 | 1.00 | 36.18 | C |
| ATOM | 4654 | CZ | PHE | A | 296 | 15.301 | −5.395 | −5.076 | 1.00 | 38.32 | C |
| ATOM | 4656 | CE2 | PHE | A | 296 | 15.172 | −5.807 | −6.332 | 1.00 | 36.15 | C |
| ATOM | 4658 | CD2 | PHE | A | 296 | 13.969 | −5.760 | −6.939 | 1.00 | 29.00 | C |
| ATOM | 4660 | C | PHE | A | 296 | 11.426 | −2.840 | −6.592 | 1.00 | 39.38 | C |
| ATOM | 4661 | O | PHE | A | 296 | 12.431 | −2.151 | −6.656 | 1.00 | 41.20 | O |
| ATOM | 4662 | N | ARG | A | 297 | 10.485 | −2.688 | −5.654 | 1.00 | 37.55 | N |
| ATOM | 4664 | CA | ARG | A | 297 | 10.624 | −1.626 | −4.641 | 1.00 | 38.59 | C |
| ATOM | 4666 | CB | ARG | A | 297 | 9.581 | −0.566 | −4.833 | 1.00 | 38.15 | C |
| ATOM | 4669 | CG | ARG | A | 297 | 8.264 | −0.977 | −4.508 | 1.00 | 37.91 | C |
| ATOM | 4672 | CD | ARG | A | 297 | 7.234 | −0.020 | −5.035 | 1.00 | 42.44 | C |
| ATOM | 4675 | NE | ARG | A | 297 | 5.889 | −0.478 | −4.612 | 1.00 | 45.80 | N |
| ATOM | 4677 | CZ | ARG | A | 297 | 4.753 | −0.271 | −5.262 | 1.00 | 40.89 | C |
| ATOM | 4678 | NH1 | ARG | A | 297 | 4.710 | −0.368 | −6.409 | 1.00 | 50.48 | N |
| ATOM | 4681 | NH2 | ARG | A | 297 | 3.650 | −0.712 | −4.767 | 1.00 | 38.31 | N |
| ATOM | 4684 | C | ARG | A | 297 | 10.560 | −1.944 | −3.197 | 1.00 | 34.05 | C |
| ATOM | 4685 | O | ARG | A | 297 | 9.901 | −2.904 | −2.761 | 1.00 | 38.82 | O |
| ATOM | 4686 | N | ILE | A | 298 | 11.224 | −1.092 | −2.432 | 1.00 | 34.02 | N |
| ATOM | 4688 | CA | ILE | A | 298 | 11.194 | −1.240 | −0.978 | 1.00 | 33.72 | C |
| ATOM | 4690 | CB | ILE | A | 298 | 12.584 | −1.357 | −0.399 | 1.00 | 35.84 | C |
| ATOM | 4692 | CG1 | ILE | A | 298 | 13.586 | −0.429 | −1.006 | 1.00 | 40.04 | C |
| ATOM | 4695 | CD1 | ILE | A | 298 | 15.017 | −0.629 | −0.399 | 1.00 | 38.54 | C |
| ATOM | 4699 | CG2 | ILE | A | 298 | 13.234 | −2.643 | −0.863 | 1.00 | 40.77 | C |
| ATOM | 4703 | C | ILE | A | 298 | 10.474 | −0.050 | −0.493 | 1.00 | 33.09 | C |
| ATOM | 4704 | O | ILE | A | 298 | 10.772 | 1.009 | −0.956 | 1.00 | 35.93 | O |
| ATOM | 4705 | N | THR | A | 299 | 9.508 | −0.206 | 0.402 | 1.00 | 38.13 | N |
| ATOM | 4707 | CA | THR | A | 299 | 8.788 | 0.958 | 0.930 | 1.00 | 39.87 | C |
| ATOM | 4709 | CB | THR | A | 299 | 7.268 | 0.829 | 0.737 | 1.00 | 41.38 | C |
| ATOM | 4711 | OG1 | THR | A | 299 | 6.955 | 0.743 | −0.658 | 1.00 | 41.84 | O |
| ATOM | 4713 | CG2 | THR | A | 299 | 6.519 | 2.079 | 1.271 | 1.00 | 38.29 | C |
| ATOM | 4717 | C | THR | A | 299 | 9.017 | 1.219 | 2.412 | 1.00 | 39.18 | C |
| ATOM | 4718 | O | THR | A | 299 | 8.719 | 0.376 | 3.182 | 1.00 | 42.23 | O |
| ATOM | 4719 | N | ILE | A | 300 | 9.561 | 2.371 | 2.818 | 1.00 | 45.04 | N |
| ATOM | 4721 | CA | ILE | A | 300 | 9.712 | 2.602 | 4.257 | 1.00 | 43.86 | C |
| ATOM | 4723 | CB | ILE | A | 300 | 11.112 | 3.118 | 4.717 | 1.00 | 43.97 | C |
| ATOM | 4725 | CG1 | ILE | A | 300 | 11.584 | 4.378 | 4.011 | 1.00 | 45.01 | C |
| ATOM | 4728 | CD1 | ILE | A | 300 | 12.548 | 5.177 | 4.939 | 1.00 | 40.08 | C |
| ATOM | 4732 | CG2 | ILE | A | 300 | 12.167 | 2.091 | 4.499 | 1.00 | 47.96 | C |
| ATOM | 4736 | C | ILE | A | 300 | 8.619 | 3.494 | 4.769 | 1.00 | 43.59 | C |
| ATOM | 4737 | O | ILE | A | 300 | 7.878 | 4.094 | 4.001 | 1.00 | 44.78 | O |
| ATOM | 4738 | N | LEU | A | 301 | 8.538 | 3.556 | 6.089 | 1.00 | 41.32 | N |
| ATOM | 4740 | CA | LEU | A | 301 | 7.515 | 4.294 | 6.798 | 1.00 | 39.77 | C |
| ATOM | 4742 | CB | LEU | A | 301 | 6.837 | 3.315 | 7.759 | 1.00 | 34.77 | C |
| ATOM | 4745 | CG | LEU | A | 301 | 6.052 | 2.205 | 7.119 | 1.00 | 30.44 | C |
| ATOM | 4747 | CD1 | LEU | A | 301 | 5.467 | 1.524 | 8.188 | 1.00 | 30.86 | C |
| ATOM | 4751 | CD2 | LEU | A | 301 | 4.968 | 2.698 | 6.252 | 1.00 | 34.38 | C |
| ATOM | 4755 | C | LEU | A | 301 | 8.106 | 5.431 | 7.632 | 1.00 | 39.51 | C |
| ATOM | 4756 | O | LEU | A | 301 | 9.292 | 5.472 | 7.850 | 1.00 | 44.43 | O |
| ATOM | 4757 | N | PRO | A | 302 | 7.286 | 6.338 | 8.142 | 1.00 | 38.79 | N |
| ATOM | 4758 | CA | PRO | A | 302 | 7.819 | 7.393 | 9.001 | 1.00 | 36.78 | C |
| ATOM | 4760 | CB | PRO | A | 302 | 6.590 | 8.187 | 9.424 | 1.00 | 37.00 | C |
| ATOM | 4763 | CG | PRO | A | 302 | 5.410 | 7.604 | 8.714 | 1.00 | 36.36 | C |
| ATOM | 4766 | CD | PRO | A | 302 | 5.832 | 6.424 | 7.958 | 1.00 | 36.66 | C |
| ATOM | 4769 | C | PRO | A | 302 | 8.510 | 6.797 | 10.226 | 1.00 | 38.02 | C |
| ATOM | 4770 | O | PRO | A | 302 | 9.516 | 7.327 | 10.642 | 1.00 | 33.13 | O |
| ATOM | 4771 | N | GLN | A | 303 | 7.995 | 5.719 | 10.811 | 1.00 | 38.55 | N |
| ATOM | 4773 | CA | GLN | A | 303 | 8.699 | 5.129 | 11.945 | 1.00 | 36.57 | C |
| ATOM | 4775 | CB | GLN | A | 303 | 8.003 | 3.902 | 12.457 | 1.00 | 35.69 | C |
| ATOM | 4778 | CG | GLN | A | 303 | 6.751 | 4.170 | 13.195 | 1.00 | 42.25 | C |
| ATOM | 4781 | CD | GLN | A | 303 | 5.602 | 4.274 | 12.297 | 1.00 | 34.75 | C |
| ATOM | 4782 | OE1 | GLN | A | 303 | 5.772 | 4.591 | 11.130 | 1.00 | 46.30 | O |
| ATOM | 4783 | NE2 | GLN | A | 303 | 4.416 | 4.026 | 12.820 | 1.00 | 42.67 | N |
| ATOM | 4786 | C | GLN | A | 303 | 10.113 | 4.672 | 11.588 | 1.00 | 42.43 | C |
| ATOM | 4787 | O | GLN | A | 303 | 10.807 | 4.115 | 12.404 | 1.00 | 44.66 | O |
| ATOM | 4788 | N | GLN | A | 304 | 10.554 | 4.872 | 10.373 | 1.00 | 44.05 | N |
| ATOM | 4790 | CA | GLN | A | 304 | 11.919 | 4.525 | 10.089 | 1.00 | 43.84 | C |
| ATOM | 4792 | CB | GLN | A | 304 | 12.025 | 3.506 | 8.939 | 1.00 | 44.50 | C |
| ATOM | 4795 | CG | GLN | A | 304 | 11.789 | 2.069 | 9.464 | 1.00 | 40.62 | C |
| ATOM | 4798 | CD | GLN | A | 304 | 10.436 | 1.517 | 9.204 | 1.00 | 41.50 | C |
| ATOM | 4799 | OE1 | GLN | A | 304 | 9.915 | 0.808 | 10.039 | 1.00 | 46.70 | O |
| ATOM | 4800 | NE2 | GLN | A | 304 | 9.849 | 1.830 | 8.050 | 1.00 | 43.88 | N |
| ATOM | 4803 | C | GLN | A | 304 | 12.735 | 5.769 | 9.864 | 1.00 | 43.47 | C |
| ATOM | 4804 | O | GLN | A | 304 | 13.806 | 5.890 | 10.460 | 1.00 | 57.31 | O |
| ATOM | 4805 | N | TYR | A | 305 | 12.253 | 6.709 | 9.056 | 1.00 | 43.84 | N |
| ATOM | 4807 | CA | TYR | A | 305 | 13.030 | 7.931 | 8.789 | 1.00 | 42.11 | C |
| ATOM | 4809 | CB | TYR | A | 305 | 12.741 | 8.542 | 7.383 | 1.00 | 42.55 | C |
| ATOM | 4812 | CG | TYR | A | 305 | 11.329 | 9.026 | 7.065 | 1.00 | 38.35 | C |
| ATOM | 4813 | CD1 | TYR | A | 305 | 10.846 | 10.230 | 7.553 | 1.00 | 39.92 | C |
| ATOM | 4815 | CE1 | TYR | A | 305 | 9.579 | 10.679 | 7.245 | 1.00 | 38.06 | C |
| ATOM | 4817 | CZ | TYR | A | 305 | 8.745 | 9.931 | 6.432 | 1.00 | 44.35 | C |
| ATOM | 4818 | OH | TYR | A | 305 | 7.445 | 10.380 | 6.121 | 1.00 | 47.26 | O |
| ATOM | 4820 | CE2 | TYR | A | 305 | 9.206 | 8.737 | 5.934 | 1.00 | 41.55 | C |
| ATOM | 4822 | CD2 | TYR | A | 305 | 10.502 | 8.296 | 6.252 | 1.00 | 39.60 | C |
| ATOM | 4824 | C | TYR | A | 305 | 12.920 | 8.991 | 9.901 | 1.00 | 42.17 | C |
| ATOM | 4825 | O | TYR | A | 305 | 13.587 | 10.034 | 9.828 | 1.00 | 47.00 | O |
| ATOM | 4826 | N | LEU | A | 306 | 12.096 | 8.727 | 10.910 | 1.00 | 38.60 | N |
| ATOM | 4828 | CA | LEU | A | 306 | 11.947 | 9.618 | 12.065 | 1.00 | 42.86 | C |
| ATOM | 4830 | CB | LEU | A | 306 | 10.473 | 9.989 | 12.345 | 1.00 | 38.48 | C |
| ATOM | 4833 | CG | LEU | A | 306 | 9.755 | 11.015 | 11.422 | 1.00 | 39.42 | C |
| ATOM | 4835 | CD1 | LEU | A | 306 | 8.401 | 11.473 | 11.981 | 1.00 | 36.26 | C |
| ATOM | 4839 | CD2 | LEU | A | 306 | 10.561 | 12.258 | 11.132 | 1.00 | 36.89 | C |
| ATOM | 4843 | C | LEU | A | 306 | 12.538 | 8.831 | 13.236 | 1.00 | 48.77 | C |
| ATOM | 4844 | O | LEU | A | 306 | 11.887 | 7.927 | 13.807 | 1.00 | 51.36 | O |
| ATOM | 4845 | N | ARG | A | 307 | 13.770 | 9.181 | 13.589 | 1.00 | 47.61 | N |
| ATOM | 4847 | CA | ARG | A | 307 | 14.523 | 8.445 | 14.583 | 1.00 | 47.50 | C |
| ATOM | 4849 | CB | ARG | A | 307 | 15.977 | 8.359 | 14.079 | 1.00 | 46.68 | C |
| ATOM | 4852 | CG | ARG | A | 307 | 17.104 | 8.267 | 15.066 | 1.00 | 41.90 | C |
| ATOM | 4855 | CD | ARG | A | 307 | 18.410 | 8.260 | 14.346 | 1.00 | 41.76 | C |
| ATOM | 4858 | NE | ARG | A | 307 | 19.209 | 9.451 | 14.580 | 1.00 | 42.84 | N |
| ATOM | 4860 | CZ | ARG | A | 307 | 19.635 | 9.809 | 15.767 | 1.00 | 42.66 | C |
| ATOM | 4861 | NH1 | ARG | A | 307 | 19.325 | 9.068 | 16.827 | 1.00 | 44.80 | N |
| ATOM | 4864 | NH2 | ARG | A | 307 | 20.373 | 10.891 | 15.905 | 1.00 | 41.35 | N |
| ATOM | 4867 | C | ARG | A | 307 | 14.391 | 9.070 | 15.969 | 1.00 | 52.43 | C |
| ATOM | 4868 | O | ARG | A | 307 | 14.582 | 10.268 | 16.145 | 1.00 | 51.41 | O |
| ATOM | 4869 | N | PRO | A | 308 | 14.052 | 8.249 | 16.959 | 1.00 | 57.65 | N |
| ATOM | 4870 | CA | PRO | A | 308 | 13.891 | 8.738 | 18.339 | 1.00 | 55.79 | C |
| ATOM | 4872 | CB | PRO | A | 308 | 13.330 | 7.543 | 19.096 | 1.00 | 57.01 | C |
| ATOM | 4875 | CG | PRO | A | 308 | 13.086 | 6.449 | 18.086 | 1.00 | 57.54 | C |
| ATOM | 4878 | CD | PRO | A | 308 | 13.797 | 6.803 | 16.819 | 1.00 | 53.58 | C |
| ATOM | 4881 | C | PRO | A | 308 | 15.184 | 9.100 | 19.017 | 1.00 | 60.31 | C |
| ATOM | 4882 | O | PRO | A | 308 | 16.192 | 8.452 | 18.810 | 1.00 | 58.95 | O |
| ATOM | 4883 | N | VAL | A | 309 | 15.129 | 10.142 | 19.841 | 1.00 | 64.67 | N |
| ATOM | 4885 | CA | VAL | A | 309 | 16.216 | 10.523 | 20.718 | 1.00 | 59.88 | C |
| ATOM | 4887 | CB | VAL | A | 309 | 16.826 | 11.759 | 20.274 | 1.00 | 57.52 | C |
| ATOM | 4889 | CG1 | VAL | A | 309 | 16.931 | 11.787 | 18.790 | 1.00 | 58.97 | C |
| ATOM | 4893 | CG2 | VAL | A | 309 | 15.968 | 12.873 | 20.747 | 1.00 | 60.32 | C |
| ATOM | 4897 | C | VAL | A | 309 | 15.562 | 10.936 | 22.006 | 1.00 | 63.55 | C |
| ATOM | 4898 | O | VAL | A | 309 | 14.369 | 11.169 | 22.030 | 1.00 | 65.36 | O |
| ATOM | 4899 | N | GLU | A | 310 | 16.327 | 11.070 | 23.074 | 1.00 | 73.08 | N |
| ATOM | 4901 | CA | GLU | A | 310 | 15.760 | 11.539 | 24.338 | 1.00 | 80.08 | C |
| ATOM | 4903 | CB | GLU | A | 310 | 16.753 | 11.379 | 25.475 | 1.00 | 81.78 | C |
| ATOM | 4906 | CG | GLU | A | 310 | 16.107 | 11.808 | 26.784 | 1.00 | 86.56 | C |
| ATOM | 4909 | CD | GLU | A | 310 | 14.633 | 11.391 | 26.845 | 1.00 | 90.07 | C |
| ATOM | 4910 | OE1 | GLU | A | 310 | 14.305 | 10.227 | 26.469 | 1.00 | 87.88 | O |
| ATOM | 4911 | OE2 | GLU | A | 310 | 13.795 | 12.227 | 27.261 | 1.00 | 91.15 | O |
| ATOM | 4912 | C | GLU | A | 310 | 15.310 | 13.005 | 24.359 | 1.00 | 82.88 | C |
| ATOM | 4913 | O | GLU | A | 310 | 16.115 | 13.897 | 24.106 | 1.00 | 83.61 | O |
| ATOM | 4914 | N | ASP | A | 311 | 14.039 | 13.256 | 24.686 | 1.00 | 86.59 | N |
| ATOM | 4916 | CA | ASP | A | 311 | 13.540 | 14.636 | 24.758 | 1.00 | 88.79 | C |
| ATOM | 4918 | CB | ASP | A | 311 | 12.066 | 14.716 | 25.204 | 1.00 | 89.65 | C |
| ATOM | 4921 | CG | ASP | A | 311 | 11.606 | 16.165 | 25.563 | 1.00 | 89.46 | C |
| ATOM | 4922 | OD1 | ASP | A | 311 | 12.281 | 17.161 | 25.205 | 1.00 | 89.51 | O |
| ATOM | 4923 | OD2 | ASP | A | 311 | 10.557 | 16.393 | 26.211 | 1.00 | 88.07 | O |
| ATOM | 4924 | C | ASP | A | 311 | 14.414 | 15.373 | 25.737 | 1.00 | 89.45 | C |
| ATOM | 4925 | O | ASP | A | 311 | 14.741 | 14.867 | 26.813 | 1.00 | 87.75 | O |
| ATOM | 4926 | N | VAL | A | 312 | 14.782 | 16.582 | 25.353 | 1.00 | 93.56 | N |
| ATOM | 4928 | CA | VAL | A | 312 | 15.717 | 17.361 | 26.138 | 1.00 | 96.45 | C |
| ATOM | 4930 | CB | VAL | A | 312 | 15.534 | 18.893 | 25.873 | 1.00 | 95.52 | C |
| ATOM | 4932 | CG1 | VAL | A | 312 | 16.767 | 19.678 | 26.302 | 1.00 | 96.45 | C |
| ATOM | 4936 | CG2 | VAL | A | 312 | 15.280 | 19.146 | 24.380 | 1.00 | 95.45 | C |
| ATOM | 4940 | C | VAL | A | 312 | 15.614 | 16.978 | 27.629 | 1.00 | 98.94 | C |
| ATOM | 4941 | O | VAL | A | 312 | 16.541 | 16.375 | 28.178 | 1.00 | 100.66 | O |
| ATOM | 4942 | N | ALA | A | 313 | 14.480 | 17.277 | 28.263 | 1.00 | 101.21 | N |
| ATOM | 4944 | CA | ALA | A | 313 | 14.360 | 17.156 | 29.723 | 1.00 | 101.91 | C |
| ATOM | 4946 | CB | ALA | A | 313 | 13.530 | 18.320 | 30.281 | 1.00 | 102.46 | C |
| ATOM | 4950 | C | ALA | A | 313 | 13.798 | 15.850 | 30.241 | 1.00 | 103.33 | C |
| ATOM | 4951 | O | ALA | A | 313 | 13.100 | 15.844 | 31.264 | 1.00 | 103.69 | O |
| ATOM | 4952 | N | THR | A | 314 | 14.094 | 14.747 | 29.550 | 1.00 | 103.85 | N |
| ATOM | 4954 | CA | THR | A | 314 | 13.656 | 13.428 | 30.005 | 1.00 | 102.71 | C |
| ATOM | 4956 | CB | THR | A | 314 | 14.576 | 12.945 | 31.136 | 1.00 | 103.86 | C |
| ATOM | 4958 | OG1 | THR | A | 314 | 14.595 | 13.910 | 32.195 | 1.00 | 101.42 | O |
| ATOM | 4960 | CG2 | THR | A | 314 | 16.027 | 12.918 | 30.655 | 1.00 | 104.20 | C |
| ATOM | 4964 | C | THR | A | 314 | 12.227 | 13.662 | 30.456 | 1.00 | 101.12 | C |
| ATOM | 4965 | O | THR | A | 314 | 11.733 | 13.087 | 31.435 | 1.00 | 99.40 | O |
| ATOM | 4966 | N | SER | A | 315 | 11.600 | 14.554 | 29.681 | 1.00 | 100.26 | N |
| ATOM | 4968 | CA | SER | A | 315 | 10.272 | 15.102 | 29.911 | 1.00 | 97.93 | C |
| ATOM | 4970 | CB | SER | A | 315 | 10.176 | 16.571 | 29.468 | 1.00 | 98.22 | C |
| ATOM | 4973 | OG | SER | A | 315 | 9.684 | 16.683 | 28.145 | 1.00 | 97.07 | O |
| ATOM | 4975 | C | SER | A | 315 | 9.481 | 14.178 | 29.038 | 1.00 | 94.99 | C |
| ATOM | 4976 | O | SER | A | 315 | 10.031 | 13.632 | 28.084 | 1.00 | 94.86 | O |
| ATOM | 4977 | N | GLN | A | 316 | 8.199 | 14.003 | 29.309 | 1.00 | 92.82 | N |
| ATOM | 4979 | CA | GLN | A | 316 | 7.575 | 12.760 | 28.827 | 1.00 | 92.34 | C |
| ATOM | 4981 | CB | GLN | A | 316 | 6.562 | 12.202 | 29.847 | 1.00 | 93.31 | C |
| ATOM | 4984 | CG | GLN | A | 316 | 7.167 | 11.182 | 30.838 | 1.00 | 96.96 | C |
| ATOM | 4987 | CD | GLN | A | 316 | 6.165 | 10.697 | 31.905 | 1.00 | 100.71 | C |
| ATOM | 4988 | OE1 | GLN | A | 316 | 4.983 | 11.049 | 31.855 | 1.00 | 104.74 | O |
| ATOM | 4989 | NE2 | GLN | A | 316 | 6.641 | 9.900 | 32.862 | 1.00 | 96.70 | N |
| ATOM | 4992 | C | GLN | A | 316 | 6.981 | 12.858 | 27.449 | 1.00 | 86.40 | C |
| ATOM | 4993 | O | GLN | A | 316 | 5.893 | 12.387 | 27.200 | 1.00 | 87.16 | O |
| ATOM | 4994 | N | ASP | A | 317 | 7.727 | 13.465 | 26.549 | 1.00 | 81.89 | N |
| ATOM | 4996 | CA | ASP | A | 317 | 7.241 | 13.689 | 25.213 | 1.00 | 82.26 | C |
| ATOM | 4998 | CB | ASP | A | 317 | 7.243 | 15.194 | 24.971 | 1.00 | 84.30 | C |
| ATOM | 5001 | CG | ASP | A | 317 | 6.705 | 15.965 | 26.178 | 1.00 | 89.01 | C |
| ATOM | 5002 | OD1 | ASP | A | 317 | 5.730 | 15.458 | 26.802 | 1.00 | 91.04 | O |
| ATOM | 5003 | OD2 | ASP | A | 317 | 7.188 | 17.060 | 26.578 | 1.00 | 87.88 | O |
| ATOM | 5004 | C | ASP | A | 317 | 8.072 | 12.925 | 24.173 | 1.00 | 78.89 | C |
| ATOM | 5005 | O | ASP | A | 317 | 9.277 | 13.134 | 24.036 | 1.00 | 81.83 | O |
| ATOM | 5006 | N | ASP | A | 318 | 7.428 | 12.025 | 23.439 | 1.00 | 73.81 | N |
| ATOM | 5008 | CA | ASP | A | 318 | 8.155 | 11.262 | 22.451 | 1.00 | 66.66 | C |
| ATOM | 5010 | CB | ASP | A | 318 | 7.241 | 10.236 | 21.763 | 1.00 | 64.21 | C |
| ATOM | 5013 | CG | ASP | A | 318 | 6.808 | 9.090 | 22.706 | 1.00 | 64.94 | C |
| ATOM | 5014 | OD1 | ASP | A | 318 | 5.736 | 8.463 | 22.486 | 1.00 | 68.15 | O |
| ATOM | 5015 | OD2 | ASP | A | 318 | 7.470 | 8.740 | 23.698 | 1.00 | 61.61 | O |
| ATOM | 5016 | C | ASP | A | 318 | 8.666 | 12.307 | 21.485 | 1.00 | 61.08 | C |
| ATOM | 5017 | O | ASP | A | 318 | 7.859 | 12.973 | 20.848 | 1.00 | 60.70 | O |
| ATOM | 5018 | N | CYS | A | 319 | 9.990 | 12.464 | 21.414 | 1.00 | 55.63 | N |
| ATOM | 5020 | CA | CYS | A | 319 | 10.655 | 13.418 | 20.497 | 1.00 | 55.86 | C |
| ATOM | 5022 | CB | CYS | A | 319 | 11.595 | 14.339 | 21.302 | 1.00 | 56.79 | C |
| ATOM | 5025 | SG | CYS | A | 319 | 10.863 | 15.611 | 22.418 | 1.00 | 63.91 | S |
| ATOM | 5026 | C | CYS | A | 319 | 11.458 | 12.689 | 19.373 | 1.00 | 51.54 | C |
| ATOM | 5027 | O | CYS | A | 319 | 11.816 | 11.533 | 19.532 | 1.00 | 51.66 | O |
| ATOM | 5028 | N | TYR | A | 320 | 11.761 | 13.350 | 18.252 | 1.00 | 52.64 | N |
| ATOM | 5030 | CA | TYR | A | 320 | 12.442 | 12.673 | 17.126 | 1.00 | 52.76 | C |
| ATOM | 5032 | CB | TYR | A | 320 | 11.419 | 11.973 | 16.190 | 1.00 | 54.35 | C |
| ATOM | 5035 | CG | TYR | A | 320 | 10.480 | 10.999 | 16.836 | 1.00 | 48.89 | C |
| ATOM | 5036 | CD1 | TYR | A | 320 | 9.245 | 11.395 | 17.280 | 1.00 | 51.58 | C |
| ATOM | 5038 | CE1 | TYR | A | 320 | 8.367 | 10.513 | 17.879 | 1.00 | 51.79 | C |
| ATOM | 5040 | CZ | TYR | A | 320 | 8.724 | 9.195 | 18.030 | 1.00 | 59.86 | C |
| ATOM | 5041 | OH | TYR | A | 320 | 7.873 | 8.275 | 18.620 | 1.00 | 65.37 | O |
| ATOM | 5043 | CE2 | TYR | A | 320 | 9.952 | 8.770 | 17.589 | 1.00 | 61.76 | C |
| ATOM | 5045 | CD2 | TYR | A | 320 | 10.828 | 9.691 | 16.988 | 1.00 | 58.71 | C |
| ATOM | 5047 | C | TYR | A | 320 | 13.207 | 13.621 | 16.224 | 1.00 | 49.27 | C |
| ATOM | 5048 | O | TYR | A | 320 | 12.864 | 14.785 | 16.151 | 1.00 | 48.00 | O |
| ATOM | 5049 | N | LYS | A | 321 | 14.208 | 13.094 | 15.517 | 1.00 | 47.06 | N |
| ATOM | 5051 | CA | LYS | A | 321 | 15.025 | 13.851 | 14.547 | 1.00 | 49.46 | C |
| ATOM | 5053 | CB | LYS | A | 321 | 16.515 | 13.796 | 14.895 | 1.00 | 48.67 | C |
| ATOM | 5056 | CG | LYS | A | 321 | 17.182 | 15.141 | 15.141 | 1.00 | 53.31 | C |
| ATOM | 5059 | CD | LYS | A | 321 | 18.714 | 15.093 | 14.895 | 1.00 | 53.64 | C |
| ATOM | 5062 | CE | LYS | A | 321 | 19.429 | 16.349 | 15.512 | 1.00 | 55.80 | C |
| ATOM | 5065 | NZ | LYS | A | 321 | 20.917 | 16.178 | 15.790 | 1.00 | 57.93 | N |
| ATOM | 5069 | C | LYS | A | 321 | 14.857 | 13.255 | 13.163 | 1.00 | 43.54 | C |
| ATOM | 5070 | O | LYS | A | 321 | 14.681 | 12.069 | 13.016 | 1.00 | 49.60 | O |
| ATOM | 5071 | N | PHE | A | 322 | 14.947 | 14.097 | 12.161 | 1.00 | 41.25 | N |
| ATOM | 5073 | CA | PHE | A | 322 | 14.655 | 13.750 | 10.791 | 1.00 | 43.43 | C |
| ATOM | 5075 | CB | PHE | A | 322 | 14.199 | 15.061 | 10.075 | 1.00 | 45.65 | C |
| ATOM | 5078 | CG | PHE | A | 322 | 13.807 | 14.910 | 8.629 | 1.00 | 41.86 | C |
| ATOM | 5079 | CD1 | PHE | A | 322 | 12.758 | 14.134 | 8.257 | 1.00 | 41.79 | C |
| ATOM | 5081 | CE1 | PHE | A | 322 | 12.427 | 14.000 | 6.960 | 1.00 | 37.29 | C |
| ATOM | 5083 | CZ | PHE | A | 322 | 13.125 | 14.653 | 5.982 | 1.00 | 40.05 | C |
| ATOM | 5085 | CE2 | PHE | A | 322 | 14.158 | 15.436 | 6.310 | 1.00 | 42.54 | C |
| ATOM | 5087 | CD2 | PHE | A | 322 | 14.509 | 15.566 | 7.642 | 1.00 | 46.49 | C |
| ATOM | 5089 | C | PHE | A | 322 | 15.961 | 13.138 | 10.239 | 1.00 | 48.97 | C |
| ATOM | 5090 | O | PHE | A | 322 | 16.981 | 13.817 | 10.014 | 1.00 | 48.91 | O |
| ATOM | 5091 | N | ALA | A | 323 | 15.922 | 11.831 | 10.036 | 1.00 | 42.08 | N |
| ATOM | 5093 | CA | ALA | A | 323 | 17.072 | 11.115 | 9.597 | 1.00 | 39.45 | C |
| ATOM | 5095 | CB | ALA | A | 323 | 17.011 | 9.753 | 10.228 | 1.00 | 38.16 | C |
| ATOM | 5099 | C | ALA | A | 323 | 17.297 | 11.012 | 8.047 | 1.00 | 42.70 | C |
| ATOM | 5100 | O | ALA | A | 323 | 17.863 | 10.006 | 7.562 | 1.00 | 40.41 | O |
| ATOM | 5101 | N | ILE | A | 324 | 16.914 | 12.027 | 7.268 | 1.00 | 39.06 | N |
| ATOM | 5103 | CA | ILE | A | 324 | 17.177 | 11.971 | 5.799 | 1.00 | 40.46 | C |
| ATOM | 5105 | CB | ILE | A | 324 | 15.911 | 12.049 | 4.976 | 1.00 | 35.33 | C |
| ATOM | 5107 | CG1 | ILE | A | 324 | 15.063 | 10.826 | 5.158 | 1.00 | 41.82 | C |
| ATOM | 5110 | CD1 | ILE | A | 324 | 13.800 | 10.854 | 4.283 | 1.00 | 45.15 | C |
| ATOM | 5114 | CG2 | ILE | A | 324 | 16.241 | 12.092 | 3.553 | 1.00 | 39.72 | C |
| ATOM | 5118 | C | ILE | A | 324 | 18.000 | 13.179 | 5.453 | 1.00 | 41.45 | C |
| ATOM | 5119 | O | ILE | A | 324 | 17.491 | 14.268 | 5.571 | 1.00 | 47.75 | O |
| ATOM | 5120 | N | SER | A | 325 | 19.244 | 13.022 | 5.011 | 1.00 | 45.67 | N |
| ATOM | 5122 | CA | SER | A | 325 | 20.148 | 14.181 | 4.894 | 1.00 | 47.83 | C |
| ATOM | 5124 | CB | SER | A | 325 | 21.194 | 14.129 | 5.977 | 1.00 | 51.02 | C |
| ATOM | 5127 | OG | SER | A | 325 | 21.991 | 12.978 | 5.786 | 1.00 | 60.15 | O |
| ATOM | 5129 | C | SER | A | 325 | 20.831 | 14.230 | 3.573 | 1.00 | 44.39 | C |
| ATOM | 5130 | O | SER | A | 325 | 20.699 | 13.316 | 2.780 | 1.00 | 51.62 | O |
| ATOM | 5131 | N | GLN | A | 326 | 21.579 | 15.286 | 3.320 | 1.00 | 49.08 | N |
| ATOM | 5133 | CA | GLN | A | 326 | 22.095 | 15.527 | 1.954 | 1.00 | 50.41 | C |
| ATOM | 5135 | CB | GLN | A | 326 | 21.980 | 17.004 | 1.575 | 1.00 | 50.65 | C |
| ATOM | 5138 | CG | GLN | A | 326 | 22.818 | 17.900 | 2.410 | 1.00 | 59.27 | C |
| ATOM | 5141 | CD | GLN | A | 326 | 22.308 | 19.336 | 2.379 | 1.00 | 69.60 | C |
| ATOM | 5142 | OE1 | GLN | A | 326 | 22.805 | 20.165 | 1.608 | 1.00 | 81.67 | O |
| ATOM | 5143 | NE2 | GLN | A | 326 | 21.315 | 19.636 | 3.215 | 1.00 | 74.53 | N |
| ATOM | 5146 | C | GLN | A | 326 | 23.522 | 15.036 | 1.838 | 1.00 | 47.73 | C |
| ATOM | 5147 | O | GLN | A | 326 | 24.139 | 14.823 | 2.858 | 1.00 | 39.28 | O |
| ATOM | 5148 | N | SER | A | 327 | 24.036 | 14.838 | 0.616 | 1.00 | 50.50 | N |
| ATOM | 5150 | CA | SER | A | 327 | 25.349 | 14.191 | 0.407 | 1.00 | 48.29 | C |
| ATOM | 5152 | CB | SER | A | 327 | 25.170 | 12.660 | 0.588 | 1.00 | 50.33 | C |
| ATOM | 5155 | OG | SER | A | 327 | 26.109 | 11.808 | −0.130 | 1.00 | 44.83 | O |
| ATOM | 5157 | C | SER | A | 327 | 26.057 | 14.402 | −0.934 | 1.00 | 54.98 | C |
| ATOM | 5158 | O | SER | A | 327 | 25.440 | 14.385 | −2.005 | 1.00 | 60.63 | O |
| ATOM | 5159 | N | SER | A | 328 | 27.375 | 14.538 | −0.843 | 1.00 | 58.53 | N |
| ATOM | 5161 | CA | SER | A | 328 | 28.228 | 14.891 | −1.967 | 1.00 | 59.21 | C |
| ATOM | 5163 | CB | SER | A | 328 | 29.294 | 15.862 | −1.479 | 1.00 | 59.74 | C |
| ATOM | 5166 | OG | SER | A | 328 | 30.068 | 15.264 | −0.446 | 1.00 | 58.39 | O |
| ATOM | 5168 | C | SER | A | 328 | 28.901 | 13.650 | −2.507 | 1.00 | 63.61 | C |
| ATOM | 5169 | O | SER | A | 328 | 29.458 | 13.656 | −3.630 | 1.00 | 62.54 | O |
| ATOM | 5170 | N | THR | A | 329 | 28.861 | 12.588 | −1.696 | 1.00 | 60.68 | N |
| ATOM | 5172 | CA | THR | A | 329 | 29.372 | 11.305 | −2.132 | 1.00 | 58.73 | C |
| ATOM | 5174 | CB | THR | A | 329 | 30.254 | 10.683 | −1.073 | 1.00 | 59.15 | C |
| ATOM | 5176 | OG1 | THR | A | 329 | 29.635 | 10.794 | 0.212 | 1.00 | 56.80 | O |
| ATOM | 5178 | CG2 | THR | A | 329 | 31.542 | 11.477 | −0.972 | 1.00 | 61.68 | C |
| ATOM | 5182 | C | THR | A | 329 | 28.280 | 10.328 | −2.543 | 1.00 | 55.97 | C |
| ATOM | 5183 | O | THR | A | 329 | 28.473 | 9.113 | −2.470 | 1.00 | 61.73 | O |
| ATOM | 5184 | N | GLY | A | 330 | 27.131 | 10.843 | −2.965 | 1.00 | 52.78 | N |
| ATOM | 5186 | CA | GLY | A | 330 | 26.102 | 9.994 | −3.558 | 1.00 | 47.81 | C |
| ATOM | 5189 | C | GLY | A | 330 | 25.072 | 9.508 | −2.565 | 1.00 | 40.66 | C |
| ATOM | 5190 | O | GLY | A | 330 | 25.057 | 9.994 | −1.446 | 1.00 | 41.70 | O |
| ATOM | 5191 | N | THR | A | 331 | 24.242 | 8.545 | −2.962 | 1.00 | 37.29 | N |
| ATOM | 5193 | CA | THR | A | 331 | 23.170 | 8.072 | −2.094 | 1.00 | 37.86 | C |
| ATOM | 5195 | CB | THR | A | 331 | 22.068 | 7.313 | −2.904 | 1.00 | 39.26 | C |
| ATOM | 5197 | OG1 | THR | A | 331 | 21.479 | 8.139 | −3.916 | 1.00 | 39.31 | O |
| ATOM | 5199 | CG2 | THR | A | 331 | 20.888 | 7.049 | −2.051 | 1.00 | 42.31 | C |
| ATOM | 5203 | C | THR | A | 331 | 23.756 | 7.124 | −1.078 | 1.00 | 39.73 | C |
| ATOM | 5204 | O | THR | A | 331 | 24.660 | 6.353 | −1.407 | 1.00 | 44.56 | O |
| ATOM | 5205 | N | VAL | A | 332 | 23.258 | 7.178 | 0.154 | 1.00 | 40.26 | N |
| ATOM | 5207 | CA | VAL | A | 332 | 23.641 | 6.187 | 1.152 | 1.00 | 35.90 | C |
| ATOM | 5209 | CB | VAL | A | 332 | 24.604 | 6.690 | 2.229 | 1.00 | 40.57 | C |
| ATOM | 5211 | CG1 | VAL | A | 332 | 24.912 | 5.525 | 3.187 | 1.00 | 39.73 | C |
| ATOM | 5215 | CG2 | VAL | A | 332 | 25.881 | 7.177 | 1.612 | 1.00 | 41.22 | C |
| ATOM | 5219 | C | VAL | A | 332 | 22.424 | 5.582 | 1.853 | 1.00 | 42.39 | C |
| ATOM | 5220 | O | VAL | A | 332 | 21.729 | 6.293 | 2.599 | 1.00 | 41.60 | O |
| ATOM | 5221 | N | MET | A | 333 | 22.203 | 4.285 | 1.594 | 1.00 | 37.82 | N |
| ATOM | 5223 | CA | MET | A | 333 | 21.117 | 3.471 | 2.170 | 1.00 | 36.93 | C |
| ATOM | 5225 | CB | MET | A | 333 | 20.723 | 2.317 | 1.246 | 1.00 | 37.72 | C |
| ATOM | 5228 | CG | MET | A | 333 | 20.166 | 2.782 | −0.085 | 1.00 | 41.56 | C |
| ATOM | 5231 | SD | MET | A | 333 | 19.579 | 1.455 | −1.132 | 1.00 | 50.56 | S |
| ATOM | 5232 | CE | MET | A | 333 | 18.013 | 1.284 | −0.388 | 1.00 | 55.20 | C |
| ATOM | 5236 | C | MET | A | 333 | 21.588 | 2.919 | 3.503 | 1.00 | 41.46 | C |
| ATOM | 5237 | O | MET | A | 333 | 22.205 | 1.777 | 3.611 | 1.00 | 39.46 | O |
| ATOM | 5238 | N | GLY | A | 334 | 21.291 | 3.762 | 4.508 | 1.00 | 40.49 | N |
| ATOM | 5240 | CA | GLY | A | 334 | 21.812 | 3.648 | 5.846 | 1.00 | 38.05 | C |
| ATOM | 5243 | C | GLY | A | 334 | 20.941 | 2.902 | 6.789 | 1.00 | 38.82 | C |
| ATOM | 5244 | O | GLY | A | 334 | 19.921 | 2.335 | 6.417 | 1.00 | 44.15 | O |
| ATOM | 5245 | N | ALA | A | 335 | 21.359 | 2.914 | 8.045 | 1.00 | 38.69 | N |
| ATOM | 5247 | CA | ALA | A | 335 | 20.658 | 2.169 | 9.044 | 1.00 | 38.89 | C |
| ATOM | 5249 | CB | ALA | A | 335 | 21.021 | 2.632 | 10.403 | 1.00 | 39.90 | C |
| ATOM | 5253 | C | ALA | A | 335 | 19.193 | 2.369 | 8.825 | 1.00 | 42.45 | C |
| ATOM | 5254 | O | ALA | A | 335 | 18.353 | 1.432 | 8.827 | 1.00 | 41.48 | O |
| ATOM | 5255 | N | VAL | A | 336 | 18.830 | 3.606 | 8.611 | 1.00 | 43.44 | N |
| ATOM | 5257 | CA | VAL | A | 336 | 17.425 | 3.755 | 8.643 | 1.00 | 46.66 | C |
| ATOM | 5259 | CB | VAL | A | 336 | 16.973 | 5.203 | 8.808 | 1.00 | 49.40 | C |
| ATOM | 5261 | CG1 | VAL | A | 336 | 17.623 | 5.737 | 10.156 | 1.00 | 55.39 | C |
| ATOM | 5265 | CG2 | VAL | A | 336 | 17.340 | 6.070 | 7.645 | 1.00 | 52.52 | C |
| ATOM | 5269 | C | VAL | A | 336 | 16.921 | 2.865 | 7.543 | 1.00 | 46.80 | C |
| ATOM | 5270 | O | VAL | A | 336 | 16.221 | 1.924 | 7.810 | 1.00 | 48.30 | O |
| ATOM | 5271 | N | ILE | A | 337 | 17.318 | 3.049 | 6.309 | 1.00 | 45.47 | N |
| ATOM | 5273 | CA | ILE | A | 337 | 16.677 | 2.216 | 5.338 | 1.00 | 36.74 | C |
| ATOM | 5275 | CB | ILE | A | 337 | 17.298 | 2.390 | 3.978 | 1.00 | 39.78 | C |
| ATOM | 5277 | CG1 | ILE | A | 337 | 17.151 | 3.822 | 3.500 | 1.00 | 36.27 | C |
| ATOM | 5280 | CD1 | ILE | A | 337 | 15.779 | 4.199 | 3.294 | 1.00 | 43.14 | C |
| ATOM | 5284 | CG2 | ILE | A | 337 | 16.671 | 1.450 | 2.978 | 1.00 | 34.84 | C |
| ATOM | 5288 | C | ILE | A | 337 | 16.885 | 0.804 | 5.806 | 1.00 | 39.51 | C |
| ATOM | 5289 | O | ILE | A | 337 | 16.013 | −0.096 | 5.623 | 1.00 | 35.70 | O |
| ATOM | 5290 | N | MET | A | 338 | 18.031 | 0.511 | 6.397 | 1.00 | 38.60 | N |
| ATOM | 5292 | CA | MET | A | 338 | 18.215 | −0.928 | 6.662 | 1.00 | 39.01 | C |
| ATOM | 5294 | CB | MET | A | 338 | 19.673 | −1.259 | 7.028 | 1.00 | 37.15 | C |
| ATOM | 5297 | CG | MET | A | 338 | 20.587 | −1.321 | 5.812 | 1.00 | 38.15 | C |
| ATOM | 5300 | SD | MET | A | 338 | 22.320 | −1.770 | 6.100 | 1.00 | 49.00 | S |
| ATOM | 5301 | CE | MET | A | 338 | 22.236 | −3.500 | 6.563 | 1.00 | 47.85 | C |
| ATOM | 5305 | C | MET | A | 338 | 17.197 | −1.470 | 7.680 | 1.00 | 36.06 | C |
| ATOM | 5306 | O | MET | A | 338 | 16.768 | −2.596 | 7.615 | 1.00 | 40.65 | O |
| ATOM | 5307 | N | GLU | A | 339 | 16.780 | −0.644 | 8.610 | 1.00 | 43.64 | N |
| ATOM | 5309 | CA | GLU | A | 339 | 15.981 | −1.092 | 9.755 | 1.00 | 43.34 | C |
| ATOM | 5311 | CB | GLU | A | 339 | 15.922 | 0.036 | 10.811 | 1.00 | 44.54 | C |
| ATOM | 5314 | CG | GLU | A | 339 | 16.905 | −0.101 | 11.984 | 1.00 | 50.03 | C |
| ATOM | 5317 | CD | GLU | A | 339 | 17.134 | 1.179 | 12.854 | 1.00 | 50.19 | C |
| ATOM | 5318 | OE1 | GLU | A | 339 | 18.297 | 1.508 | 13.284 | 1.00 | 50.60 | O |
| ATOM | 5319 | OE2 | GLU | A | 339 | 16.170 | 1.853 | 13.133 | 1.00 | 52.29 | O |
| ATOM | 5320 | C | GLU | A | 339 | 14.593 | −1.616 | 9.375 | 1.00 | 44.44 | C |
| ATOM | 5321 | O | GLU | A | 339 | 13.825 | −2.079 | 10.235 | 1.00 | 48.83 | O |
| ATOM | 5322 | N | GLY | A | 340 | 14.257 | −1.572 | 8.096 | 1.00 | 37.01 | N |
| ATOM | 5324 | CA | GLY | A | 340 | 13.003 | −2.140 | 7.664 | 1.00 | 35.04 | C |
| ATOM | 5327 | C | GLY | A | 340 | 13.074 | −3.539 | 7.035 | 1.00 | 37.57 | C |
| ATOM | 5328 | O | GLY | A | 340 | 12.067 | −4.268 | 6.954 | 1.00 | 30.82 | O |
| ATOM | 5329 | N | PHE | A | 341 | 14.251 | −3.954 | 6.584 | 1.00 | 36.28 | N |
| ATOM | 5331 | CA | PHE | A | 341 | 14.284 | −5.201 | 5.860 | 1.00 | 37.94 | C |
| ATOM | 5333 | CB | PHE | A | 341 | 14.549 | −4.833 | 4.467 | 1.00 | 35.78 | C |
| ATOM | 5336 | CG | PHE | A | 341 | 13.721 | −3.727 | 4.036 | 1.00 | 36.57 | C |
| ATOM | 5337 | CD1 | PHE | A | 341 | 14.141 | −2.449 | 4.214 | 1.00 | 42.48 | C |
| ATOM | 5339 | CE1 | PHE | A | 341 | 13.364 | −1.430 | 3.810 | 1.00 | 45.68 | C |
| ATOM | 5341 | CZ | PHE | A | 341 | 12.165 | −1.691 | 3.237 | 1.00 | 43.50 | C |
| ATOM | 5343 | CE2 | PHE | A | 341 | 11.735 | −2.983 | 3.058 | 1.00 | 39.45 | C |
| ATOM | 5345 | CD2 | PHE | A | 341 | 12.493 | −3.971 | 3.451 | 1.00 | 39.09 | C |
| ATOM | 5347 | C | PHE | A | 341 | 15.287 | −6.189 | 6.323 | 1.00 | 37.29 | C |
| ATOM | 5348 | O | PHE | A | 341 | 16.019 | −5.890 | 7.200 | 1.00 | 41.19 | O |
| ATOM | 5349 | N | TYR | A | 342 | 15.294 | −7.383 | 5.745 | 1.00 | 38.28 | N |
| ATOM | 5351 | CA | TYR | A | 342 | 16.295 | −8.381 | 6.079 | 1.00 | 39.65 | C |
| ATOM | 5353 | CB | TYR | A | 342 | 15.710 | −9.774 | 6.073 | 1.00 | 40.40 | C |
| ATOM | 5356 | CG | TYR | A | 342 | 16.664 | −10.890 | 6.407 | 1.00 | 41.95 | C |
| ATOM | 5357 | CD1 | TYR | A | 342 | 17.625 | −10.727 | 7.340 | 1.00 | 43.94 | C |
| ATOM | 5359 | CE1 | TYR | A | 342 | 18.488 | −11.731 | 7.651 | 1.00 | 48.72 | C |
| ATOM | 5361 | CZ | TYR | A | 342 | 18.405 | −12.947 | 7.036 | 1.00 | 49.33 | C |
| ATOM | 5362 | OH | TYR | A | 342 | 19.319 | −13.922 | 7.408 | 1.00 | 51.44 | O |
| ATOM | 5364 | CE2 | TYR | A | 342 | 17.442 | −13.159 | 6.091 | 1.00 | 43.68 | C |
| ATOM | 5366 | CD2 | TYR | A | 342 | 16.573 | −12.122 | 5.784 | 1.00 | 47.27 | C |
| ATOM | 5368 | C | TYR | A | 342 | 17.119 | −8.182 | 4.875 | 1.00 | 36.83 | C |
| ATOM | 5369 | O | TYR | A | 342 | 16.568 | −8.334 | 3.816 | 1.00 | 38.58 | O |
| ATOM | 5370 | N | VAL | A | 343 | 18.398 | −7.818 | 4.992 | 1.00 | 32.01 | N |
| ATOM | 5372 | CA | VAL | A | 343 | 19.202 | −7.554 | 3.788 | 1.00 | 31.56 | C |
| ATOM | 5374 | CB | VAL | A | 343 | 20.074 | −6.367 | 3.980 | 1.00 | 35.24 | C |
| ATOM | 5376 | CG1 | VAL | A | 343 | 20.737 | −5.970 | 2.673 | 1.00 | 32.39 | C |
| ATOM | 5380 | CG2 | VAL | A | 343 | 19.278 | −5.211 | 4.529 | 1.00 | 41.31 | C |
| ATOM | 5384 | C | VAL | A | 343 | 20.130 | −8.701 | 3.611 | 1.00 | 29.86 | C |
| ATOM | 5385 | O | VAL | A | 343 | 20.695 | −9.101 | 4.562 | 1.00 | 31.04 | O |
| ATOM | 5386 | N | VAL | A | 344 | 20.316 | −9.232 | 2.414 | 1.00 | 33.91 | N |
| ATOM | 5388 | CA | VAL | A | 344 | 21.212 | −10.337 | 2.216 | 1.00 | 28.64 | C |
| ATOM | 5390 | CB | VAL | A | 344 | 20.420 | −11.348 | 1.500 | 1.00 | 33.00 | C |
| ATOM | 5392 | CG1 | VAL | A | 344 | 21.253 | −12.622 | 1.272 | 1.00 | 39.15 | C |
| ATOM | 5396 | CG2 | VAL | A | 344 | 19.173 | −11.671 | 2.320 | 1.00 | 32.27 | C |
| ATOM | 5400 | C | VAL | A | 344 | 22.433 | −10.004 | 1.360 | 1.00 | 32.31 | C |
| ATOM | 5401 | O | VAL | A | 344 | 22.298 | −9.878 | 0.166 | 1.00 | 36.99 | O |
| ATOM | 5402 | N | PHE | A | 345 | 23.621 | −9.871 | 1.945 | 1.00 | 36.90 | N |
| ATOM | 5404 | CA | PHE | A | 345 | 24.822 | −9.592 | 1.141 | 1.00 | 36.18 | C |
| ATOM | 5406 | CB | PHE | A | 345 | 25.861 | −8.841 | 1.954 | 1.00 | 38.02 | C |
| ATOM | 5409 | CG | PHE | A | 345 | 25.449 | −7.408 | 2.249 | 1.00 | 37.44 | C |
| ATOM | 5410 | CD1 | PHE | A | 345 | 25.889 | −6.348 | 1.501 | 1.00 | 42.15 | C |
| ATOM | 5412 | CE1 | PHE | A | 345 | 25.487 | −5.139 | 1.797 | 1.00 | 40.37 | C |
| ATOM | 5414 | CZ | PHE | A | 345 | 24.621 | −4.914 | 2.850 | 1.00 | 36.09 | C |
| ATOM | 5416 | CE2 | PHE | A | 345 | 24.186 | −5.924 | 3.581 | 1.00 | 34.23 | C |
| ATOM | 5418 | CD2 | PHE | A | 345 | 24.597 | −7.177 | 3.279 | 1.00 | 32.71 | C |
| ATOM | 5420 | C | PHE | A | 345 | 25.443 | −10.824 | 0.522 | 1.00 | 36.14 | C |
| ATOM | 5421 | O | PHE | A | 345 | 26.486 | −11.317 | 0.958 | 1.00 | 39.03 | O |
| ATOM | 5422 | N | ASP | A | 346 | 24.785 | −11.298 | −0.525 | 1.00 | 35.28 | N |
| ATOM | 5424 | CA | ASP | A | 346 | 25.157 | −12.498 | −1.257 | 1.00 | 33.32 | C |
| ATOM | 5426 | CB | ASP | A | 346 | 23.865 | −12.940 | −1.938 | 1.00 | 43.05 | C |
| ATOM | 5429 | CG | ASP | A | 346 | 23.993 | −14.184 | −2.742 | 1.00 | 44.26 | C |
| ATOM | 5430 | OD1 | ASP | A | 346 | 25.122 | −14.682 | −2.972 | 1.00 | 49.09 | O |
| ATOM | 5431 | OD2 | ASP | A | 346 | 22.961 | −14.707 | −3.196 | 1.00 | 44.53 | O |
| ATOM | 5432 | C | ASP | A | 346 | 26.310 | −12.270 | −2.247 | 1.00 | 37.90 | C |
| ATOM | 5433 | O | ASP | A | 346 | 26.156 | −12.107 | −3.481 | 1.00 | 32.28 | O |
| ATOM | 5434 | N | ARG | A | 347 | 27.498 | −12.256 | −1.659 | 1.00 | 40.60 | N |
| ATOM | 5436 | CA | ARG | A | 347 | 28.764 | −12.187 | −2.385 | 1.00 | 37.22 | C |
| ATOM | 5438 | CB | ARG | A | 347 | 29.896 | −12.280 | −1.348 | 1.00 | 40.87 | C |
| ATOM | 5441 | CG | ARG | A | 347 | 29.903 | −11.047 | −0.457 | 1.00 | 34.79 | C |
| ATOM | 5444 | CD | ARG | A | 347 | 30.720 | −11.142 | 0.739 | 1.00 | 35.83 | C |
| ATOM | 5447 | NE | ARG | A | 347 | 32.086 | −11.477 | 0.381 | 1.00 | 46.81 | N |
| ATOM | 5449 | CZ | ARG | A | 347 | 33.161 | −10.768 | 0.700 | 1.00 | 48.24 | C |
| ATOM | 5450 | NH1 | ARG | A | 347 | 33.029 | −9.658 | 1.397 | 1.00 | 55.18 | N |
| ATOM | 5453 | NH2 | ARG | A | 347 | 34.372 | −11.171 | 0.317 | 1.00 | 45.55 | N |
| ATOM | 5456 | C | ARG | A | 347 | 28.920 | −13.265 | −3.429 | 1.00 | 36.71 | C |
| ATOM | 5457 | O | ARG | A | 347 | 29.227 | −12.993 | −4.573 | 1.00 | 45.36 | O |
| ATOM | 5456 | N | ALA | A | 348 | 28.699 | −14.501 | −3.041 | 1.00 | 42.24 | N |
| ATOM | 5460 | CA | ALA | A | 348 | 28.835 | −15.651 | −3.960 | 1.00 | 46.74 | C |
| ATOM | 5462 | CB | ALA | A | 348 | 28.168 | −16.883 | −3.369 | 1.00 | 49.53 | C |
| ATOM | 5466 | C | ALA | A | 348 | 28.256 | −15.440 | −5.301 | 1.00 | 43.78 | C |
| ATOM | 5467 | O | ALA | A | 348 | 28.804 | −15.893 | −6.279 | 1.00 | 51.15 | O |
| ATOM | 5468 | N | ARG | A | 349 | 27.114 | −14.772 | −5.347 | 1.00 | 50.73 | N |
| ATOM | 5470 | CA | ARG | A | 349 | 26.435 | −14.531 | −6.606 | 1.00 | 45.15 | C |
| ATOM | 5472 | CB | ARG | A | 349 | 25.008 | −15.048 | −6.522 | J..00 | 46.17 | C |
| ATOM | 5475 | CG | ARG | A | 349 | 25.021 | −16.582 | −6.566 | 1.00 | 50.92 | C |
| ATOM | 5478 | CD | ARG | A | 349 | 23.678 | −17.332 | −6.431 | 1.00 | 56.96 | C |
| ATOM | 5481 | NE | ARG | A | 349 | 22.731 | −17.167 | −7.546 | 1.00 | 58.75 | N |
| ATOM | 5483 | CZ | ARG | A | 349 | 21.525 | −17.757 | −7.576 | 1.00 | 59.56 | C |
| ATOM | 5484 | NH1 | ARG | A | 349 | 21.140 | −18.544 | −6.575 | 1.00 | 56.83 | N |
| ATOM | 5487 | NH2 | ARG | A | 349 | 20.699 | −17.576 | −8.596 | 1.00 | 57.83 | N |
| ATOM | 5490 | C | ARG | A | 349 | 26.515 | −13.088 | −7.031 | 1.00 | 46.61 | C |
| ATOM | 5491 | O | ARG | A | 349 | 26.054 | −12.752 | −8.115 | 1.00 | 48.78 | O |
| ATOM | 5492 | N | LYS | A | 350 | 27.108 | −12.227 | −6.198 | 1.00 | 43.49 | N |
| ATOM | 5494 | CA | LYS | A | 350 | 27.257 | −10.824 | −6.565 | 1.00 | 39.81 | C |
| ATOM | 5496 | CB | LYS | A | 350 | 27.981 | −10.696 | −7.901 | 1.00 | 42.63 | C |
| ATOM | 5499 | CG | LYS | A | 350 | 28.391 | −9.252 | −8.257 | 1.00 | 54.21 | C |
| ATOM | 5502 | CD | LYS | A | 350 | 29.456 | −9.124 | −9.427 | 1.00 | 58.86 | C |
| ATOM | 5505 | CE | LYS | A | 350 | 29.595 | −7.616 | −9.867 | 1.00 | 62.75 | C |
| ATOM | 5508 | NZ | LYS | A | 350 | 30.563 | −7.261 | −10.973 | 1.00 | 61.28 | N |
| ATOM | 5512 | C | LYS | A | 350 | 25.878 | −10.169 | −6.591 | 1.00 | 42.26 | C |
| ATOM | 5513 | O | LYS | A | 350 | 25.434 | −9.629 | −7.620 | 1.00 | 36.83 | O |
| ATOM | 5514 | N | ARG | A | 351 | 25.198 | −10.229 | −5.431 | 1.00 | 43.25 | N |
| ATOM | 5516 | CA | ARG | A | 351 | 23.853 | −9.649 | −5.285 | 1.00 | 39.90 | C |
| ATOM | 5518 | CB | ARG | A | 351 | 22.823 | −10.612 | −5.867 | 1.00 | 38.47 | C |
| ATOM | 5521 | CG | ARG | A | 351 | 22.635 | −11.942 | −5.173 | 1.00 | 40.60 | C |
| ATOM | 5524 | CD | ARG | A | 351 | 21.811 | −12.900 | −6.063 | 1.00 | 43.93 | C |
| ATOM | 5527 | NE | ARG | A | 351 | 21.546 | −14.178 | −5.418 | 1.00 | 50.64 | N |
| ATOM | 5529 | CZ | ARG | A | 351 | 20.398 | −14.872 | −5.490 | 1.00 | 44.16 | C |
| ATOM | 5530 | NH1 | ARG | A | 351 | 19.376 | −14.454 | −6.184 | 1.00 | 30.21 | N |
| ATOM | 5533 | NH2 | ARG | A | 351 | 20.298 | −16.009 | −4.839 | 1.00 | 45.65 | N |
| ATOM | 5536 | C | ARG | A | 351 | 23.449 | −9.270 | −3.872 | 1.00 | 34.43 | C |
| ATOM | 5537 | O | ARG | A | 351 | 23.928 | −9.841 | −2.946 | 1.00 | 40.67 | O |
| ATOM | 5538 | N | ILE | A | 352 | 22.562 | −8.292 | −3.711 | 1.00 | 34.43 | N |
| ATOM | 5540 | CA | ILE | A | 352 | 21.995 | −7.958 | −2.397 | 1.00 | 28.97 | C |
| ATOM | 5542 | CB | ILE | A | 352 | 22.232 | −6.539 | −2.051 | 1.00 | 30.73 | C |
| ATOM | 5544 | CG1 | ILE | A | 352 | 23.729 | −6.288 | −2.019 | 1.00 | 31.97 | C |
| ATOM | 5547 | CD1 | ILE | A | 352 | 24.027 | −4.969 | −2.366 | 1.00 | 34.88 | C |
| ATOM | 5551 | CG2 | ILE | A | 352 | 21.688 | −6.255 | −0.674 | 1.00 | 32.36 | C |
| ATOM | 5555 | C | ILE | A | 352 | 20.527 | −8.181 | −2.397 | 1.00 | 31.19 | C |
| ATOM | 5556 | O | ILE | A | 352 | 19.848 | −7.740 | −3.309 | 1.00 | 31.73 | O |
| ATOM | 5557 | N | GLY | A | 353 | 20.032 | −8.875 | −1.372 | 1.00 | 36.35 | N |
| ATOM | 5559 | CA | GLY | A | 353 | 18.629 | −9.239 | −1.311 | 1.00 | 37.89 | C |
| ATOM | 5562 | C | GLY | A | 353 | 17.850 | −8.451 | −0.291 | 1.00 | 38.37 | C |
| ATOM | 5563 | O | GLY | A | 353 | 18.393 | −8.137 | 0.759 | 1.00 | 42.00 | O |
| ATOM | 5564 | N | PHE | A | 354 | 16.586 | −8.143 | −0.592 | 1.00 | 37.07 | N |
| ATOM | 5566 | CA | PHE | A | 354 | 15.730 | −7.427 | 0.352 | 1.00 | 31.53 | C |
| ATOM | 5568 | CB | PHE | A | 354 | 15.379 | −5.999 | −0.164 | 1.00 | 29.79 | C |
| ATOM | 5571 | CG | PHE | A | 354 | 16.557 | −5.063 | −0.308 | 1.00 | 26.28 | C |
| ATOM | 5572 | CD1 | PHE | A | 354 | 17.323 | −5.078 | −1.450 | 1.00 | 32.33 | C |
| ATOM | 5574 | CE1 | PHE | A | 354 | 18.424 | −4.209 | −1.598 | 1.00 | 34.84 | C |
| ATOM | 5576 | CZ | PHE | A | 354 | 18.742 | −3.320 | −0.581 | 1.00 | 35.73 | C |
| ATOM | 5578 | CE2 | PHE | A | 354 | 17.971 | −3.287 | 0.571 | 1.00 | 27.61 | C |
| ATOM | 5580 | CD2 | PHE | A | 354 | 16.885 | −4.148 | 0.704 | 1.00 | 28.09 | C |
| ATOM | 5582 | C | PHE | A | 354 | 14.415 | −8.191 | 0.649 | 1.00 | 31.75 | C |
| ATOM | 5583 | O | PHE | A | 354 | 13.752 | −8.665 | −0.273 | 1.00 | 35.59 | O |
| ATOM | 5584 | N | ALA | A | 355 | 14.065 | −8.308 | 1.934 | 1.00 | 26.00 | N |
| ATOM | 5586 | CA | ALA | A | 355 | 12.707 | −8.676 | 2.345 | 1.00 | 25.47 | C |
| ATOM | 5588 | CB | ALA | A | 355 | 12.652 | −10.035 | 2.579 | 1.00 | 28.36 | C |
| ATOM | 5592 | C | ALA | A | 355 | 12.222 | −7.961 | 3.602 | 1.00 | 30.38 | C |
| ATOM | 5593 | O | ALA | A | 355 | 13.009 | −7.387 | 4.333 | 1.00 | 31.09 | O |
| ATOM | 5594 | N | VAL | A | 356 | 10.912 | −7.991 | 3.860 | 1.00 | 35.80 | N |
| ATOM | 5596 | CA | VAL | A | 356 | 10.326 | −7.206 | 4.958 | 1.00 | 37.43 | C |
| ATOM | 5598 | CB | VAL | A | 356 | 8.800 | −7.269 | 4.990 | 1.00 | 36.49 | C |
| ATOM | 5600 | CG1 | VAL | A | 356 | 8.258 | −6.199 | 5.891 | 1.00 | 41.38 | C |
| ATOM | 5604 | CG2 | VAL | A | 356 | 8.253 | −7.092 | 3.695 | 1.00 | 35.72 | C |
| ATOM | 5608 | C | VAL | A | 356 | 10.735 | −7.852 | 6.243 | 1.00 | 41.52 | C |
| ATOM | 5609 | O | VAL | A | 356 | 10.683 | −9.066 | 6.331 | 1.00 | 37.93 | O |
| ATOM | 5610 | N | SER | A | 357 | 11.104 | −7.051 | 7.242 | 1.00 | 43.38 | N |
| ATOM | 5612 | CA | SER | A | 357 | 11.551 | −7.584 | 8.506 | 1.00 | 39.52 | C |
| ATOM | 5614 | CB | SER | A | 357 | 12.327 | −6.514 | 9.235 | 1.00 | 45.00 | C |
| ATOM | 5617 | OG | SER | A | 357 | 13.156 | −7.090 | 10.258 | 1.00 | 55.64 | O |
| ATOM | 5619 | C | SER | A | 357 | 10.410 | −8.000 | 9.368 | 1.00 | 45.19 | C |
| ATOM | 5620 | O | SER | A | 357 | 9.356 | −7.417 | 9.314 | 1.00 | 52.71 | O |
| ATOM | 5621 | N | ALA | A | 358 | 10.599 | −9.023 | 10.193 | 1.00 | 51.62 | N |
| ATOM | 5623 | CA | ALA | A | 358 | 9.558 | −9.402 | 11.143 | 1.00 | 47.19 | C |
| ATOM | 5625 | CB | ALA | A | 358 | 9.455 | −10.874 | 11.270 | 1.00 | 44.33 | C |
| ATOM | 5629 | C | ALA | A | 358 | 9.858 | −8.814 | 12.486 | 1.00 | 53.26 | C |
| ATOM | 5630 | O | ALA | A | 358 | 9.324 | −9.302 | 13.472 | 1.00 | 58.72 | O |
| ATOM | 5631 | N | CYS | A | 359 | 10.720 | −7.790 | 12.540 | 1.00 | 54.45 | N |
| ATOM | 5633 | CA | CYS | A | 359 | 10.962 | −7.065 | 13.779 | 1.00 | 52.85 | C |
| ATOM | 5635 | CB | CYS | A | 359 | 12.325 | −7.455 | 14.429 | 1.00 | 55.72 | C |
| ATOM | 5638 | SG | CYS | A | 359 | 13.911 | −6.785 | 13.755 | 1.00 | 65.10 | S |
| ATOM | 5639 | C | CYS | A | 359 | 10.793 | −5.543 | 13.583 | 1.00 | 53.76 | C |
| ATOM | 5640 | O | CYS | A | 359 | 11.047 | −4.789 | 14.519 | 1.00 | 60.53 | O |
| ATOM | 5641 | N | HIS | A | 360 | 10.332 | −5.075 | 12.412 | 1.00 | 45.54 | N |
| ATOM | 5643 | CA | HIS | A | 360 | 10.265 | −3.631 | 12.161 | 1.00 | 38.19 | C |
| ATOM | 5645 | CB | HIS | A | 360 | 10.274 | −3.291 | 10.655 | 1.00 | 31.06 | C |
| ATOM | 5648 | CG | HIS | A | 360 | 8.951 | −3.409 | 9.972 | 1.00 | 34.23 | C |
| ATOM | 5649 | ND1 | HIS | A | 360 | 8.422 | −4.617 | 9.566 | 1.00 | 43.85 | N |
| ATOM | 5651 | CE1 | HIS | A | 360 | 7.259 | −4.425 | 8.979 | 1.00 | 30.40 | C |
| ATOM | 5653 | NE2 | HIS | A | 360 | 7.010 | −3.130 | 8.992 | 1.00 | 36.44 | N |
| ATOM | 5655 | CD2 | HIS | A | 360 | 8.051 | −2.475 | 9.607 | 1.00 | 29.91 | C |
| ATOM | 5657 | C | HIS | A | 360 | 9.170 | −2.820 | 12.921 | 1.00 | 41.87 | C |
| ATOM | 5658 | O | HIS | A | 360 | 8.085 | −3.281 | 13.248 | 1.00 | 47.13 | O |
| ATOM | 5659 | N | VAL | A | 361 | 9.488 | −1.576 | 13.194 | 1.00 | 36.30 | N |
| ATOM | 5661 | CA | VAL | A | 361 | 8.626 | −0.768 | 13.971 | 1.00 | 36.06 | C |
| ATOM | 5663 | CB | VAL | A | 361 | 9.448 | 0.396 | 14.601 | 1.00 | 33.69 | C |
| ATOM | 5665 | CG1 | VAL | A | 361 | 8.519 | 1.388 | 15.203 | 1.00 | 33.28 | C |
| ATOM | 5669 | CG2 | VAL | A | 361 | 10.371 | −0.136 | 15.590 | 1.00 | 30.28 | C |
| ATOM | 5673 | C | VAL | A | 361 | 7.498 | −0.219 | 13.100 | 1.00 | 35.37 | C |
| ATOM | 5674 | O | VAL | A | 361 | 7.749 | 0.397 | 12.095 | 1.00 | 28.73 | O |
| ATOM | 5675 | N | HIS | A | 362 | 6.262 | −0.453 | 13.489 | 1.00 | 38.93 | N |
| ATOM | 5677 | CA | HIS | A | 362 | 5.119 | 0.046 | 12.726 | 1.00 | 43.25 | C |
| ATOM | 5679 | CB | HIS | A | 362 | 4.939 | −0.771 | 11.462 | 1.00 | 43.67 | C |
| ATOM | 5682 | CG | HIS | A | 362 | 4.555 | −2.203 | 11.705 | 1.00 | 46.38 | C |
| ATOM | 5683 | ND1 | HIS | A | 362 | 5.479 | −3.210 | 11.850 | 1.00 | 49.03 | N |
| ATOM | 5685 | CE1 | HIS | A | 362 | 4.855 | −4.363 | 12.037 | 1.00 | 50.56 | C |
| ATOM | 5687 | NE2 | HIS | A | 362 | 3.558 | −4.143 | 12.013 | 1.00 | 45.23 | N |
| ATOM | 5689 | CD2 | HIS | A | 362 | 3.345 | −2.799 | 11.810 | 1.00 | 51.21 | C |
| ATOM | 5691 | C | HIS | A | 362 | 3.831 | 0.124 | 13.587 | 1.00 | 44.78 | C |
| ATOM | 5692 | O | HIS | A | 362 | 3.871 | −0.147 | 14.749 | 1.00 | 49.93 | O |
| ATOM | 5693 | N | ASP | A | 363 | 2.704 | 0.524 | 13.028 | 1.00 | 48.91 | N |
| ATOM | 5695 | CA | ASP | A | 363 | 1.412 | 0.508 | 13.729 | 1.00 | 48.67 | C |
| ATOM | 5697 | CB | ASP | A | 363 | 0.779 | 1.835 | 13.601 | 1.00 | 46.80 | C |
| ATOM | 5700 | CG | ASP | A | 363 | 0.798 | 2.252 | 12.190 | 1.00 | 56.05 | C |
| ATOM | 5701 | OD1 | ASP | A | 363 | 0.064 | 1.570 | 11.427 | 1.00 | 62.04 | O |
| ATOM | 5702 | OD2 | ASP | A | 363 | 1.530 | 3.170 | 11.732 | 1.00 | 53.10 | O |
| ATOM | 5703 | C | ASP | A | 363 | 0.583 | −0.390 | 12.839 | 1.00 | 50.55 | C |
| ATOM | 5704 | O | ASP | A | 363 | 1.082 | −0.877 | 11.824 | 1.00 | 49.82 | O |
| ATOM | 5705 | N | GLU | A | 364 | −0.698 | −0.564 | 13.161 | 1.00 | 51.52 | N |
| ATOM | 5707 | CA | GLU | A | 364 | −1.506 | −1.526 | 12.443 | 1.00 | 50.07 | C |
| ATOM | 5709 | CB | GLU | A | 364 | −2.705 | −1.964 | 13.242 | 1.00 | 54.11 | C |
| ATOM | 5712 | CG | GLU | A | 364 | −3.727 | −0.871 | 13.464 | 1.00 | 56.03 | C |
| ATOM | 5715 | CD | GLU | A | 364 | −5.070 | −1.412 | 13.986 | 1.00 | 64.46 | C |
| ATOM | 5716 | OE1 | GLU | A | 364 | −6.040 | −0.598 | 13.967 | 1.00 | 76.03 | O |
| ATOM | 5717 | OE2 | GLU | A | 364 | −5.182 | −2.618 | 14.399 | 1.00 | 46.64 | O |
| ATOM | 5718 | C | GLU | A | 364 | −1.989 | −1.015 | 11.144 | 1.00 | 47.66 | C |
| ATOM | 5719 | O | GLU | A | 364 | −2.524 | −1.783 | 10.362 | 1.00 | 47.48 | O |
| ATOM | 5720 | N | PHE | A | 365 | −1.799 | 0.259 | 10.884 | 1.00 | 41.55 | N |
| ATOM | 5722 | CA | PHE | A | 365 | −2.309 | 0.801 | 9.651 | 1.00 | 44.15 | C |
| ATOM | 5724 | CB | PHE | A | 365 | −2.603 | 2.213 | 9.951 | 1.00 | 42.84 | C |
| ATOM | 5727 | CG | PHE | A | 365 | −3.640 | 2.376 | 10.974 | 1.00 | 42.11 | C |
| ATOM | 5728 | CD1 | PHE | A | 365 | −3.391 | 3.047 | 12.126 | 1.00 | 43.74 | C |
| ATOM | 5730 | CE1 | PHE | A | 365 | −4.332 | 3.195 | 13.033 | 1.00 | 43.17 | C |
| ATOM | 5732 | CZ | PHE | A | 365 | −5.532 | 2.685 | 12.820 | 1.00 | 47.95 | C |
| ATOM | 5734 | CE2 | PHE | A | 365 | −5.793 | 2.006 | 11.673 | 1.00 | 50.59 | C |
| ATOM | 5736 | CD2 | PHE | A | 365 | −4.869 | 1.858 | 10.771 | 1.00 | 47.37 | C |
| ATOM | 5738 | C | PHE | A | 365 | −1.462 | 0.662 | 8.321 | 1.00 | 46.25 | C |
| ATOM | 5739 | O | PHE | A | 365 | −2.031 | 0.487 | 7.227 | 1.00 | 49.35 | O |
| ATOM | 5740 | N | ARG | A | 366 | −0.143 | 0.745 | 8.428 | 1.00 | 42.85 | N |
| ATOM | 5742 | CA | ARG | A | 366 | 0.796 | 0.645 | 7.309 | 1.00 | 41.91 | C |
| ATOM | 5744 | CB | ARG | A | 366 | 1.398 | 1.997 | 6.985 | 1.00 | 40.96 | C |
| ATOM | 5747 | CG | ARG | A | 366 | 0.536 | 2.993 | 6.424 | 1.00 | 40.90 | C |
| ATOM | 5750 | CD | ARG | A | 366 | 1.341 | 4.073 | 5.811 | 1.00 | 40.88 | C |
| ATOM | 5753 | NE | ARG | A | 366 | 1.839 | 4.979 | 6.843 | 1.00 | 40.97 | N |
| ATOM | 5755 | CZ | ARG | A | 366 | 1.893 | 6.275 | 6.661 | 1.00 | 40.91 | C |
| ATOM | 5756 | NH1 | ARG | A | 366 | 1.476 | 6.743 | 5.493 | 1.00 | 36.36 | N |
| ATOM | 5759 | NH2 | ARG | A | 366 | 2.346 | 7.096 | 7.609 | 1.00 | 40.33 | N |
| ATOM | 5762 | C | ARG | A | 366 | 2.061 | −0.090 | 7.758 | 1.00 | 41.93 | C |
| ATOM | 5763 | O | ARG | A | 366 | 2.491 | 0.129 | 8.894 | 1.00 | 41.59 | O |
| ATOM | 5764 | N | THR | A | 367 | 2.677 | −0.865 | 6.847 | 1.00 | 37.72 | N |
| ATOM | 5766 | CA | THR | A | 367 | 3.839 | −1.681 | 7.138 | 1.00 | 33.65 | C |
| ATOM | 5768 | CB | THR | A | 367 | 3.469 | −3.107 | 7.143 | 1.00 | 42.32 | C |
| ATOM | 5770 | OG1 | THR | A | 367 | 4.646 | −3.854 | 6.891 | 1.00 | 43.84 | O |
| ATOM | 5772 | CG2 | THR | A | 367 | 2.528 | −3.460 | 5.969 | 1.00 | 43.37 | C |
| ATOM | 5776 | C | THR | A | 367 | 4.827 | −1.507 | 6.066 | 1.00 | 32.72 | C |
| ATOM | 5777 | O | THR | A | 367 | 4.465 | −1.157 | 4.970 | 1.00 | 34.98 | O |
| ATOM | 5778 | N | ALA | A | 368 | 6.110 | −1.702 | 6.359 | 1.00 | 39.92 | N |
| ATOM | 5780 | CA | ALA | A | 368 | 7.135 | −1.504 | 5.307 | 1.00 | 40.50 | C |
| ATOM | 5782 | CB | ALA | A | 368 | 8.485 | −1.608 | 5.870 | 1.00 | 38.72 | C |
| ATOM | 5786 | C | ALA | A | 368 | 6.910 | −2.529 | 4.180 | 1.00 | 43.33 | C |
| ATOM | 5787 | O | ALA | A | 368 | 6.181 | −3.498 | 4.382 | 1.00 | 47.27 | O |
| ATOM | 5788 | N | ALA | A | 369 | 7.494 | −2.369 | 2.998 | 1.00 | 41.85 | N |
| ATOM | 5790 | CA | ALA | A | 369 | 7.115 | −3.346 | 1.966 | 1.00 | 38.56 | C |
| ATOM | 5792 | CB | ALA | A | 369 | 5.832 | −2.891 | 1.277 | 1.00 | 40.40 | C |
| ATOM | 5796 | C | ALA | A | 369 | 8.162 | −3.635 | 0.928 | 1.00 | 29.85 | C |
| ATOM | 5797 | O | ALA | A | 369 | 9.095 | −2.854 | 0.730 | 1.00 | 36.35 | O |
| ATOM | 5798 | N | VAL | A | 370 | 7.966 | −4.769 | 0.274 | 1.00 | 25.96 | N |
| ATOM | 5800 | CA | VAL | A | 370 | 8.831 | −5.260 | −0.769 | 1.00 | 29.57 | C |
| ATOM | 5802 | CB | VAL | A | 370 | 9.849 | −6.185 | −0.229 | 1.00 | 26.20 | C |
| ATOM | 5804 | CG1 | VAL | A | 370 | 10.757 | −6.632 | −1.321 | 1.00 | 32.44 | C |
| ATOM | 5808 | CG2 | VAL | A | 370 | 10.689 | −5.482 | 0.827 | 1.00 | 30.95 | C |
| ATOM | 5812 | C | VAL | A | 370 | 7.929 | −5.941 | −1.796 | 1.00 | 31.92 | C |
| ATOM | 5813 | O | VAL | A | 370 | 7.225 | −6.866 | −1.490 | 1.00 | 36.53 | O |
| ATOM | 5814 | N | GLU | A | 371 | 7.945 | −5.452 | −3.029 | 1.00 | 33.29 | N |
| ATOM | 5816 | CA | GLU | A | 371 | 6.979 | −5.876 | −3.989 | 1.00 | 28.61 | C |
| ATOM | 5818 | CB | GLU | A | 371 | 5.769 | −4.939 | −3.899 | 1.00 | 32.04 | C |
| ATOM | 5821 | CG | GLU | A | 371 | 5.266 | −4.723 | −2.470 | 1.00 | 38.80 | C |
| ATOM | 5824 | CD | GLU | A | 371 | 4.374 | −3.505 | −2.287 | 1.00 | 44.41 | C |
| ATOM | 5825 | OE1 | GLU | A | 371 | 3.119 | −3.685 | −2.230 | 1.00 | 55.91 | O |
| ATOM | 5826 | OE2 | GLU | A | 371 | 4.919 | −2.370 | −2.177 | 1.00 | 45.34 | O |
| ATOM | 5827 | C | GLU | A | 371 | 7.625 | −5.792 | −5.338 | 1.00 | 34.43 | C |
| ATOM | 5828 | O | GLU | A | 371 | 8.335 | −4.857 | −5.587 | 1.00 | 31.86 | O |
| ATOM | 5829 | N | GLY | A | 372 | 7.410 | −6.797 | −6.201 | 1.00 | 41.45 | N |
| ATOM | 5831 | CA | GLY | A | 372 | 7.862 | −6.730 | −7.585 | 1.00 | 44.38 | C |
| ATOM | 5834 | C | GLY | A | 372 | 7.151 | −7.793 | −8.402 | 1.00 | 49.60 | C |
| ATOM | 5835 | O | GLY | A | 372 | 6.383 | −8.580 | −7.851 | 1.00 | 48.56 | O |
| ATOM | 5836 | N | PRO | A | 373 | 7.383 | −7.862 | −9.709 | 1.00 | 50.59 | N |
| ATOM | 5837 | CA | PRO | A | 373 | 8.248 | −6.984 | −10.495 | 1.00 | 49.61 | C |
| ATOM | 5839 | CB | PRO | A | 373 | 8.726 | −7.915 | −11.588 | 1.00 | 51.13 | C |
| ATOM | 5842 | CG | PRO | A | 373 | 7.732 | −9.025 | −11.666 | 1.00 | 50.04 | C |
| ATOM | 5845 | CD | PRO | A | 373 | 6.799 | −8.918 | −10.531 | 1.00 | 50.00 | C |
| ATOM | 5848 | C | PRO | A | 373 | 7.502 | −5.877 | −11.212 | 1.00 | 51.84 | C |
| ATOM | 5849 | O | PRO | A | 373 | 6.324 | −6.033 | −11.428 | 1.00 | 59.43 | O |
| ATOM | 5850 | N | PHE | A | 374 | 8.177 | −4.797 | −11.584 | 1.00 | 53.98 | N |
| ATOM | 5852 | CA | PHE | A | 374 | 7.578 | −3.700 | −12.360 | 1.00 | 55.44 | C |
| ATOM | 5854 | CB | PHE | A | 374 | 7.709 | −2.369 | −11.615 | 1.00 | 53.01 | C |
| ATOM | 5857 | CG | PHE | A | 374 | 7.163 | −2.396 | −10.227 | 1.00 | 51.03 | C |
| ATOM | 5858 | CD1 | PHE | A | 374 | 7.959 | −2.743 | −9.179 | 1.00 | 47.22 | C |
| ATOM | 5860 | CE1 | PHE | A | 374 | 7.448 | −2.766 | −7.895 | 1.00 | 52.78 | C |
| ATOM | 5862 | CZ | PHE | A | 374 | 6.132 | −2.437 | −7.669 | 1.00 | 52.67 | C |
| ATOM | 5864 | CE2 | PHE | A | 374 | 5.326 | −2.083 | −8.737 | 1.00 | 49.05 | C |
| ATOM | 5866 | CD2 | PHE | A | 374 | 5.840 | −2.063 | −9.987 | 1.00 | 49.61 | C |
| ATOM | 5868 | C | PHE | A | 374 | 8.342 | −3.543 | −13.656 | 1.00 | 57.87 | C |
| ATOM | 5869 | O | PHE | A | 374 | 9.536 | −3.729 | −13.651 | 1.00 | 59.75 | O |
| ATOM | 5870 | N | VAL | A | 375 | 7.692 | −3.183 | −14.762 | 1.00 | 68.38 | N |
| ATOM | 5872 | CA | VAL | A | 375 | 8.440 | −3.007 | −16.020 | 1.00 | 72.14 | C |
| ATOM | 5874 | CB | VAL | A | 375 | 7.752 | −3.656 | −17.217 | 1.00 | 73.03 | C |
| ATOM | 5876 | CG1 | VAL | A | 375 | 8.601 | −3.444 | −18.453 | 1.00 | 71.54 | C |
| ATOM | 5880 | CG2 | VAL | A | 375 | 7.514 | −5.160 | −16.966 | 1.00 | 72.18 | C |
| ATOM | 5884 | C | VAL | A | 375 | 8.757 | −1.541 | −16.347 | 1.00 | 76.96 | C |
| ATOM | 5885 | O | VAL | A | 375 | 7.909 | −0.792 | −16.840 | 1.00 | 79.60 | O |
| ATOM | 5886 | N | THR | A | 376 | 10.008 | −1.161 | −16.073 | 1.00 | 85.14 | N |
| ATOM | 5888 | CA | THR | A | 376 | 10.529 | 0.219 | −16.248 | 1.00 | 88.23 | C |
| ATOM | 5890 | CB | THR | A | 376 | 11.255 | 0.740 | −14.901 | 1.00 | 89.17 | C |
| ATOM | 5892 | OG1 | THR | A | 376 | 11.057 | −0.171 | −13.804 | 1.00 | 90.48 | O |
| ATOM | 5894 | CG2 | THR | A | 376 | 10.674 | 1.992 | −14.352 | 1.00 | 86.17 | C |
| ATOM | 5898 | C | THR | A | 376 | 11.560 | 0.063 | −17.389 | 1.00 | 88.70 | C |
| ATOM | 5899 | O | THR | A | 376 | 11.864 | −1.086 | −17.791 | 1.00 | 87.38 | O |
| ATOM | 5900 | N | LEU | A | 377 | 12.114 | 1.155 | −17.922 | 1.00 | 87.83 | N |
| ATOM | 5902 | CA | LEU | A | 377 | 13.111 | 0.969 | −18.996 | 1.00 | 88.50 | C |
| ATOM | 5904 | CB | LEU | A | 377 | 12.510 | 1.234 | −20.409 | 1.00 | 90.35 | C |
| ATOM | 5907 | CG | LEU | A | 377 | 11.904 | 0.243 | −21.438 | 1.00 | 89.96 | C |
| ATOM | 5909 | CD1 | LEU | A | 377 | 12.034 | 0.935 | −22.783 | 1.00 | 89.80 | C |
| ATOM | 5913 | CD2 | LEU | A | 377 | 12.520 | −1.159 | −21.579 | 1.00 | 90.81 | C |
| ATOM | 5917 | C | LEU | A | 377 | 14.475 | 1.712 | −18.873 | 1.00 | 89.75 | C |
| ATOM | 5918 | O | LEU | A | 377 | 14.792 | 2.450 | −17.889 | 1.00 | 82.52 | O |
| ATOM | 5919 | N | ASP | A | 378 | 15.258 | 1.480 | −19.937 | 1.00 | 92.45 | N |
| ATOM | 5921 | CA | ASP | A | 378 | 16.640 | 1.894 | −20.006 | 1.00 | 95.71 | C |
| ATOM | 5923 | CB | ASP | A | 378 | 17.018 | 2.767 | −21.238 | 1.00 | 98.28 | C |
| ATOM | 5926 | CG | ASP | A | 378 | 17.513 | 1.894 | −22.480 | 1.00 | 102.00 | C |
| ATOM | 5927 | OD1 | ASP | A | 378 | 17.062 | 2.143 | −23.628 | 1.00 | 101.56 | O |
| ATOM | 5928 | OD2 | ASP | A | 378 | 18.342 | 0.937 | −22.402 | 1.00 | 102.38 | O |
| ATOM | 5929 | C | ASP | A | 378 | 16.922 | 2.405 | −18.610 | 1.00 | 94.33 | C |
| ATOM | 5930 | O | ASP | A | 378 | 17.057 | 3.589 | −18.295 | 1.00 | 96.66 | O |
| ATOM | 5931 | N | MET | A | 379 | 16.945 | 1.359 | −17.798 | 1.00 | 91.54 | N |
| ATOM | 5933 | CA | MET | A | 379 | 17.353 | 1.328 | −16.436 | 1.00 | 87.87 | C |
| ATOM | 5935 | CB | MET | A | 379 | 16.910 | −0.019 | −15.863 | 1.00 | 83.99 | C |
| ATOM | 5938 | CG | MET | A | 379 | 15.410 | −0.082 | −15.499 | 1.00 | 78.36 | C |
| ATOM | 5941 | SD | MET | A | 379 | 15.163 | −1.374 | −14.259 | 1.00 | 66.38 | S |
| ATOM | 5942 | CE | MET | A | 379 | 15.285 | −2.746 | −15.258 | 1.00 | 67.19 | C |
| ATOM | 5946 | C | MET | A | 379 | 18.885 | 1.495 | −16.353 | 1.00 | 89.93 | C |
| ATOM | 5947 | O | MET | A | 379 | 19.336 | 2.602 | −15.998 | 1.00 | 89.20 | O |
| ATOM | 5948 | N | GLU | A | 380 | 19.666 | 0.432 | −16.667 | 1.00 | 90.93 | N |
| ATOM | 5950 | CA | GLU | A | 380 | 21.180 | 0.455 | −16.602 | 1.00 | 93.24 | C |
| ATOM | 5952 | CB | GLU | A | 380 | 21.820 | −0.615 | −17.575 | 1.00 | 92.65 | C |
| ATOM | 5955 | CG | GLU | A | 380 | 23.205 | −1.231 | −17.244 | 1.00 | 91.21 | C |
| ATOM | 5958 | CD | GLU | A | 380 | 23.355 | −1.819 | −15.836 | 1.00 | 93.45 | C |
| ATOM | 5959 | OE1 | GLU | A | 380 | 23.037 | −3.027 | −15.615 | 1.00 | 84.68 | O |
| ATOM | 5960 | OE2 | GLU | A | 380 | 23.811 | −1.058 | −14.941 | 1.00 | 94.42 | O |
| ATOM | 5961 | C | GLU | A | 380 | 21.762 | 1.922 | −16.785 | 1.00 | 93.77 | C |
| ATOM | 5962 | O | GLU | A | 380 | 22.744 | 2.305 | −16.108 | 1.00 | 94.04 | O |
| ATOM | 5963 | N | ASP | A | 381 | 21.142 | 2.717 | −17.684 | 1.00 | 92.40 | N |
| ATOM | 5965 | CA | ASP | A | 381 | 21.428 | 4.156 | −17.845 | 1.00 | 89.14 | C |
| ATOM | 5967 | CB | ASP | A | 381 | 20.206 | 4.883 | −18.410 | 1.00 | 90.34 | C |
| ATOM | 5970 | CG | ASP | A | 381 | 19.817 | 4.397 | −19.774 | 1.00 | 96.59 | C |
| ATOM | 5971 | OD1 | ASP | A | 381 | 20.728 | 4.002 | −20.536 | 1.00 | 101.01 | O |
| ATOM | 5972 | OD2 | ASP | A | 381 | 18.627 | 4.380 | −20.175 | 1.00 | 96.62 | O |
| ATOM | 5973 | C | ASP | A | 381 | 21.691 | 4.860 | −16.531 | 1.00 | 83.94 | C |
| ATOM | 5974 | O | ASP | A | 381 | 22.594 | 5.688 | −16.416 | 1.00 | 87.02 | O |
| ATOM | 5975 | N | CYS | A | 382 | 20.883 | 4.521 | −15.540 | 1.00 | 74.79 | N |
| ATOM | 5977 | CA | CYS | A | 382 | 20.891 | 5.209 | −14.273 | 1.00 | 66.78 | C |
| ATOM | 5979 | CB | CYS | A | 382 | 19.874 | 4.543 | −13.367 | 1.00 | 67.02 | C |
| ATOM | 5982 | SG | CYS | A | 382 | 18.314 | 4.339 | −14.255 | 1.00 | 65.45 | S |
| ATOM | 5983 | C | CYS | A | 382 | 22.273 | 5.125 | −13.730 | 1.00 | 62.15 | C |
| ATOM | 5984 | O | CYS | A | 382 | 22.585 | 5.663 | −12.700 | 1.00 | 59.92 | O |
| ATOM | 5985 | N | GLY | A | 383 | 23.123 | 4.433 | −14.447 | 1.00 | 61.83 | N |
| ATOM | 5987 | CA | GLY | A | 383 | 24.478 | 4.336 | −14.011 | 1.00 | 66.26 | C |
| ATOM | 5990 | C | GLY | A | 383 | 25.143 | 5.670 | −14.225 | 1.00 | 69.13 | C |
| ATOM | 5991 | O | GLY | A | 383 | 25.005 | 6.281 | −15.266 | 1.00 | 70.50 | O |
| ATOM | 5992 | N | TYR | A | 384 | 25.854 | 6.144 | −13.226 | 1.00 | 73.69 | N |
| ATOM | 5994 | CA | TYR | A | 384 | 26.709 | 7.277 | −13.454 | 1.00 | 77.96 | C |
| ATOM | 5996 | CB | TYR | A | 384 | 26.793 | 8.188 | −12.245 | 1.00 | 81.61 | C |
| ATOM | 5999 | CG | TYR | A | 384 | 28.007 | 9.089 | −12.283 | 1.00 | 85.12 | C |
| ATOM | 6000 | CD1 | TYR | A | 384 | 27.941 | 10.364 | −12.854 | 1.00 | 88.82 | C |
| ATOM | 6002 | CE1 | TYR | A | 384 | 29.048 | 11.194 | −12.893 | 1.00 | 87.19 | C |
| ATOM | 6004 | CZ | TYR | A | 384 | 30.239 | 10.752 | −12.358 | 1.00 | 90.20 | C |
| ATOM | 6005 | OH | TYR | A | 384 | 31.364 | 11.553 | −12.375 | 1.00 | 89.10 | O |
| ATOM | 6007 | CE2 | TYR | A | 384 | 30.320 | 9.490 | −11.790 | 1.00 | 90.57 | C |
| ATOM | 6009 | CD2 | TYR | A | 384 | 29.208 | 8.670 | −11.757 | 1.00 | 84.83 | C |
| ATOM | 6011 | C | TYR | A | 384 | 28.056 | 6.649 | −13.746 | 1.00 | 77.71 | C |
| ATOM | 6012 | O | TYR | A | 384 | 28.429 | 5.664 | −13.131 | 1.00 | 75.56 | O |
| ATOM | 6013 | N | ASN | A | 385 | 28.780 | 7.220 | −14.691 | 1.00 | 80.96 | N |
| ATOM | 6015 | CA | ASN | A | 385 | 30.051 | 6.662 | −15.125 | 1.00 | 82.76 | C |
| ATOM | 6017 | CB | ASN | A | 385 | 29.941 | 6.194 | −16.592 | 1.00 | 84.72 | C |
| ATOM | 6020 | CG | ASN | A | 385 | 29.453 | 4.740 | −16.736 | 1.00 | 87.65 | C |
| ATOM | 6021 | OD1 | ASN | A | 385 | 30.172 | 3.790 | −16.404 | 1.00 | 85.93 | O |
| ATOM | 6022 | ND2 | ASN | A | 385 | 28.236 | 4.570 | −17.258 | 1.00 | 88.07 | N |
| ATOM | 6025 | C | ASN | A | 385 | 31.164 | 7.705 | −14.990 | 1.00 | 83.09 | C |
| ATOM | 6026 | O | ASN | A | 385 | 30.936 | 8.930 | −14.965 | 1.00 | 81.57 | O |
| ATOM | 6027 | OXT | ASN | A | 385 | 32.333 | 7.337 | −14.908 | 1.00 | 82.17 | O |
| ATOM | 6028 | N | SER | B | −2 | 27.206 | 72.118 | 43.679 | 1.00 | 55.48 | N |
| ATOM | 6030 | CA | SER | B | −2 | 27.761 | 73.467 | 43.412 | 1.00 | 51.63 | C |
| ATOM | 6032 | CB | SER | B | −2 | 29.098 | 73.250 | 42.712 | 1.00 | 52.68 | C |
| ATOM | 6035 | OG | SER | B | −2 | 29.217 | 71.863 | 42.380 | 1.00 | 49.83 | O |
| ATOM | 6037 | C | SER | B | −2 | 26.787 | 74.421 | 42.623 | 1.00 | 54.62 | C |
| ATOM | 6038 | O | SER | B | −2 | 26.955 | 75.629 | 42.639 | 1.00 | 56.38 | O |
| ATOM | 6041 | N | PHE | B | −1 | 25.784 | 73.900 | 41.916 | 1.00 | 57.53 | N |
| ATOM | 6043 | CA | PHE | B | −1 | 24.690 | 74.760 | 41.393 | 1.00 | 57.72 | C |
| ATOM | 6045 | CB | PHE | B | −1 | 24.532 | 74.456 | 39.920 | 1.00 | 56.01 | C |
| ATOM | 6048 | CG | PHE | B | −1 | 25.795 | 74.655 | 39.141 | 1.00 | 51.92 | C |
| ATOM | 6049 | CD1 | PHE | B | −1 | 26.898 | 73.854 | 39.389 | 1.00 | 50.49 | C |
| ATOM | 6051 | CE1 | PHE | B | −1 | 28.085 | 74.025 | 38.686 | 1.00 | 55.85 | C |
| ATOM | 6053 | CZ | PHE | B | −1 | 28.179 | 75.017 | 37.725 | 1.00 | 54.01 | C |
| ATOM | 6055 | CE2 | PHE | B | −1 | 27.077 | 75.840 | 37.470 | 1.00 | 55.50 | C |
| ATOM | 6057 | CD2 | PHE | B | −1 | 25.888 | 75.657 | 38.182 | 1.00 | 50.57 | C |
| ATOM | 6059 | C | PHE | B | −1 | 23.294 | 74.634 | 42.061 | 1.00 | 61.06 | C |
| ATOM | 6060 | O | PHE | B | −1 | 22.332 | 74.340 | 41.364 | 1.00 | 68.12 | O |
| ATOM | 6061 | N | VAL | B | 0 | 23.176 | 74.848 | 43.380 | 1.00 | 62.33 | N |
| ATOM | 6063 | CA | VAL | B | 0 | 21.896 | 74.751 | 44.116 | 1.00 | 58.16 | C |
| ATOM | 6065 | CB | VAL | B | 0 | 21.965 | 75.382 | 45.528 | 1.00 | 61.92 | C |
| ATOM | 6067 | CG1 | VAL | B | 0 | 22.579 | 74.434 | 46.564 | 1.00 | 61.26 | C |
| ATOM | 6071 | CG2 | VAL | B | 0 | 22.697 | 76.717 | 45.480 | 1.00 | 64.85 | C |
| ATOM | 6075 | C | VAL | B | 0 | 20.728 | 75.507 | 43.571 | 1.00 | 59.05 | C |
| ATOM | 6076 | O | VAL | B | 0 | 19.588 | 75.100 | 43.748 | 1.00 | 64.61 | O |
| ATOM | 6077 | N | GLU | B | 1 | 20.973 | 76.632 | 42.925 | 1.00 | 61.72 | N |
| ATOM | 6079 | CA | GLU | B | 1 | 19.857 | 77.434 | 42.461 | 1.00 | 57.96 | C |
| ATOM | 6081 | CB | GLU | B | 1 | 20.322 | 78.617 | 41.603 | 1.00 | 57.40 | C |
| ATOM | 6084 | CG | GLU | B | 1 | 21.725 | 79.136 | 41.914 | 1.00 | 58.84 | C |
| ATOM | 6087 | CD | GLU | B | 1 | 22.786 | 78.540 | 41.002 | 1.00 | 60.23 | C |
| ATOM | 6088 | OE1 | GLU | B | 1 | 22.369 | 77.908 | 40.015 | 1.00 | 67.79 | O |
| ATOM | 6089 | OE2 | GLU | B | 1 | 24.023 | 78.696 | 41.244 | 1.00 | 55.89 | O |
| ATOM | 6090 | C | GLU | B | 1 | 19.026 | 76.490 | 41.626 | 1.00 | 57.15 | C |
| ATOM | 6091 | O | GLU | B | 1 | 17.785 | 76.566 | 41.632 | 1.00 | 51.98 | O |
| ATOM | 6092 | N | MET | B | 2 | 19.747 | 75.587 | 40.937 | 1.00 | 51.73 | N |
| ATOM | 6094 | CA | MET | B | 2 | 19.194 | 74.706 | 39.908 | 1.00 | 53.66 | C |
| ATOM | 6096 | CB | MET | B | 2 | 20.186 | 74.649 | 38.749 | 1.00 | 53.83 | C |
| ATOM | 6099 | CG | MET | B | 2 | 20.424 | 76.043 | 38.232 | 1.00 | 54.04 | C |
| ATOM | 6102 | SD | MET | B | 2 | 21.390 | 76.126 | 36.827 | 1.00 | 61.26 | S |
| ATOM | 6103 | CE | MET | B | 2 | 22.774 | 75.563 | 37.449 | 1.00 | 61.16 | C |
| ATOM | 6107 | C | MET | B | 2 | 18.775 | 73.312 | 40.298 | 1.00 | 53.70 | C |
| ATOM | 6108 | O | MET | B | 2 | 17.937 | 72.707 | 39.652 | 1.00 | 60.40 | O |
| ATOM | 6109 | N | VAL | B | 3 | 19.344 | 72.792 | 41.355 | 1.00 | 55.79 | N |
| ATOM | 6111 | CA | VAL | B | 3 | 18.997 | 71.464 | 41.806 | 1.00 | 54.02 | C |
| ATOM | 6113 | CB | VAL | B | 3 | 19.858 | 71.128 | 43.026 | 1.00 | 55.97 | C |
| ATOM | 6115 | CG1 | VAL | B | 3 | 19.458 | 69.812 | 43.622 | 1.00 | 56.80 | C |
| ATOM | 6119 | CG2 | VAL | B | 3 | 21.330 | 71.152 | 42.649 | 1.00 | 57.96 | C |
| ATOM | 6123 | C | VAL | B | 3 | 17.517 | 71.392 | 42.216 | 1.00 | 52.70 | C |
| ATOM | 6124 | O | VAL | B | 3 | 16.984 | 72.320 | 42.812 | 1.00 | 54.10 | O |
| ATOM | 6125 | N | ASP | B | 4 | 16.860 | 70.285 | 41.897 | 1.00 | 54.43 | N |
| ATOM | 6127 | CA | ASP | B | 4 | 15.474 | 70.048 | 42.309 | 1.00 | 58.27 | C |
| ATOM | 6129 | CB | ASP | B | 4 | 15.255 | 70.401 | 43.793 | 1.00 | 62.53 | C |
| ATOM | 6132 | CG | ASP | B | 4 | 15.485 | 69.206 | 44.726 | 1.00 | 71.31 | C |
| ATOM | 6133 | OD1 | ASP | B | 4 | 15.328 | 69.381 | 45.965 | 1.00 | 76.06 | O |
| ATOM | 6134 | OD2 | ASP | B | 4 | 15.822 | 68.060 | 44.317 | 1.00 | 75.48 | O |
| ATOM | 6135 | C | ASP | B | 4 | 14.543 | 70.851 | 41.443 | 1.00 | 53.53 | C |
| ATOM | 6136 | O | ASP | B | 4 | 13.461 | 71.276 | 41.875 | 1.00 | 51.34 | O |
| ATOM | 6137 | N | ASN | B | 5 | 14.978 | 71.054 | 40.209 | 1.00 | 50.18 | N |
| ATOM | 6139 | CA | ASN | B | 5 | 14.233 | 71.893 | 39.290 | 1.00 | 44.49 | C |
| ATOM | 6141 | CB | ASN | B | 5 | 15.122 | 72.909 | 38.651 | 1.00 | 42.71 | C |
| ATOM | 6144 | CG | ASN | B | 5 | 16.020 | 72.302 | 37.633 | 1.00 | 48.45 | C |
| ATOM | 6145 | OD1 | ASN | B | 5 | 15.983 | 71.073 | 37.445 | 1.00 | 41.96 | O |
| ATOM | 6146 | ND2 | ASN | B | 5 | 16.850 | 73.156 | 36.946 | 1.00 | 38.24 | N |
| ATOM | 6149 | C | ASN | B | 5 | 13.525 | 71.157 | 38.209 | 1.00 | 42.49 | C |
| ATOM | 6150 | O | ASN | B | 5 | 12.928 | 71.804 | 37.403 | 1.00 | 52.24 | O |
| ATOM | 6151 | N | LEU | B | 6 | 13.559 | 69.826 | 38.171 | 1.00 | 42.05 | N |
| ATOM | 6153 | CA | LEU | B | 6 | 12.673 | 69.114 | 37.261 | 1.00 | 39.02 | C |
| ATOM | 6155 | CB | LEU | B | 6 | 13.329 | 67.894 | 36.675 | 1.00 | 35.91 | C |
| ATOM | 6158 | CG | LEU | B | 6 | 14.619 | 68.130 | 35.905 | 1.00 | 35.63 | C |
| ATOM | 6160 | CD1 | LEU | B | 6 | 15.044 | 66.856 | 35.207 | 1.00 | 35.19 | C |
| ATOM | 6164 | CD2 | LEU | B | 6 | 14.462 | 69.193 | 34.893 | 1.00 | 29.54 | C |
| ATOM | 6168 | C | LEU | B | 6 | 11.446 | 68.653 | 37.996 | 1.00 | 42.53 | C |
| ATOM | 6169 | O | LEU | B | 6 | 11.394 | 68.726 | 39.205 | 1.00 | 53.16 | O |
| ATOM | 6170 | N | ARG | B | 7 | 10.436 | 68.228 | 37.245 | 1.00 | 42.75 | N |
| ATOM | 6172 | CA | ARG | B | 7 | 9.325 | 67.471 | 37.771 | 1.00 | 38.99 | C |
| ATOM | 6174 | CB | ARG | B | 7 | 8.103 | 68.326 | 37.963 | 1.00 | 41.20 | C |
| ATOM | 6177 | CG | ARG | B | 7 | 8.306 | 69.383 | 39.081 | 1.00 | 48.07 | C |
| ATOM | 6180 | CD | ARG | B | 7 | 7.066 | 70.215 | 39.470 | 1.00 | 48.13 | C |
| ATOM | 6183 | NE | ARG | B | 7 | 6.508 | 70.882 | 38.289 | 1.00 | 63.74 | N |
| ATOM | 6185 | CZ | ARG | B | 7 | 5.417 | 71.659 | 38.262 | 1.00 | 67.03 | C |
| ATOM | 6186 | NH1 | ARG | B | 7 | 4.720 | 71.907 | 39.367 | 1.00 | 73.42 | N |
| ATOM | 6189 | NH2 | ARG | B | 7 | 5.021 | 72.192 | 37.118 | 1.00 | 66.22 | N |
| ATOM | 6192 | C | ARG | B | 7 | 9.095 | 66.395 | 36.744 | 1.00 | 41.31 | C |
| ATOM | 6193 | O | ARG | B | 7 | 9.743 | 66.393 | 35.697 | 1.00 | 42.80 | O |
| ATOM | 6194 | N | GLY | B | 8 | 8.213 | 65.451 | 37.037 | 1.00 | 44.24 | N |
| ATOM | 6196 | CA | GLY | B | 8 | 7.775 | 64.510 | 36.017 | 1.00 | 44.58 | C |
| ATOM | 6199 | C | GLY | B | 8 | 8.126 | 63.080 | 36.314 | 1.00 | 52.57 | C |
| ATOM | 6200 | O | GLY | B | 8 | 8.464 | 62.734 | 37.457 | 1.00 | 54.12 | O |
| ATOM | 6201 | N | LYS | B | 9 | 8.059 | 62.251 | 35.270 | 1.00 | 55.79 | N |
| ATOM | 6203 | CA | LYS | B | 9 | 8.331 | 60.829 | 35.404 | 1.00 | 58.69 | C |
| ATOM | 6205 | CB | LYS | B | 9 | 7.136 | 60.163 | 36.102 | 1.00 | 63.35 | C |
| ATOM | 6208 | CG | LYS | B | 9 | 5.746 | 60.383 | 35.457 | 1.00 | 64.18 | C |
| ATOM | 6211 | CD | LYS | B | 9 | 4.812 | 59.234 | 35.916 | 1.00 | 65.46 | C |
| ATOM | 6214 | CE | LYS | B | 9 | 3.321 | 59.481 | 35.622 | 1.00 | 64.84 | C |
| ATOM | 6217 | NZ | LYS | B | 9 | 2.630 | 60.116 | 36.794 | 1.00 | 61.17 | N |
| ATOM | 6221 | C | LYS | B | 9 | 8.661 | 60.089 | 34.086 | 1.00 | 59.28 | C |
| ATOM | 6222 | O | LYS | B | 9 | 8.631 | 60.659 | 32.990 | 1.00 | 57.59 | O |
| ATOM | 6223 | N | SER | B | 10 | 8.974 | 58.805 | 34.211 | 1.00 | 58.73 | N |
| ATOM | 6225 | CA | SER | B | 10 | 9.357 | 57.968 | 33.073 | 1.00 | 61.81 | C |
| ATOM | 6227 | CB | SER | B | 10 | 9.764 | 56.630 | 33.631 | 1.00 | 62.78 | C |
| ATOM | 6230 | OG | SER | B | 10 | 8.858 | 56.310 | 34.679 | 1.00 | 64.36 | O |
| ATOM | 6232 | C | SER | B | 10 | 8.256 | 57.723 | 32.029 | 1.00 | 62.89 | C |
| ATOM | 6233 | O | SER | B | 10 | 8.509 | 57.692 | 30.820 | 1.00 | 63.54 | O |
| ATOM | 6234 | N | GLY | B | 11 | 7.034 | 57.522 | 32.497 | 1.00 | 60.46 | N |
| ATOM | 6236 | CA | GLY | B | 11 | 5.913 | 57.328 | 31.607 | 1.00 | 57.59 | C |
| ATOM | 6239 | C | GLY | B | 11 | 5.615 | 58.594 | 30.850 | 1.00 | 56.83 | C |
| ATOM | 6240 | O | GLY | B | 11 | 5.172 | 58.582 | 29.710 | 1.00 | 59.77 | O |
| ATOM | 6241 | N | GLN | B | 12 | 5.855 | 59.731 | 31.456 | 1.00 | 57.32 | N |
| ATOM | 6243 | CA | GLN | B | 12 | 5.495 | 60.920 | 30.714 | 1.00 | 58.21 | C |
| ATOM | 6245 | CB | GLN | B | 12 | 4.324 | 61.552 | 31.404 | 1.00 | 60.77 | C |
| ATOM | 6248 | CG | GLN | B | 12 | 3.241 | 60.485 | 31.587 | 1.00 | 61.87 | C |
| ATOM | 6251 | CD | GLN | B | 12 | 1.931 | 61.070 | 31.972 | 1.00 | 63.35 | C |
| ATOM | 6252 | OE1 | GLN | B | 12 | 1.866 | 61.968 | 32.838 | 1.00 | 64.47 | O |
| ATOM | 6253 | NE2 | GLN | B | 12 | 0.862 | 60.578 | 31.346 | 1.00 | 65.23 | N |
| ATOM | 6256 | C | GLN | B | 12 | 6.566 | 61.922 | 30.368 | 1.00 | 57.52 | C |
| ATOM | 6257 | O | GLN | B | 12 | 6.322 | 62.740 | 29.514 | 1.00 | 55.14 | O |
| ATOM | 6258 | N | GLY | B | 13 | 7.747 | 61.848 | 30.973 | 1.00 | 55.44 | N |
| ATOM | 6260 | CA | GLY | B | 13 | 8.778 | 62.837 | 30.689 | 1.00 | 52.72 | C |
| ATOM | 6263 | C | GLY | B | 13 | 9.039 | 63.777 | 31.852 | 1.00 | 51.10 | C |
| ATOM | 6264 | O | GLY | B | 13 | 8.229 | 63.885 | 32.783 | 1.00 | 47.37 | O |
| ATOM | 6265 | N | TYR | B | 14 | 10.180 | 64.450 | 31.775 | 1.00 | 47.23 | N |
| ATOM | 6267 | CA | TYR | B | 14 | 10.685 | 65.341 | 32.818 | 1.00 | 48.86 | C |
| ATOM | 6269 | CB | TYR | B | 14 | 12.079 | 64.874 | 33.266 | 1.00 | 47.74 | C |
| ATOM | 6272 | CG | TYR | B | 14 | 12.061 | 63.570 | 34.037 | 1.00 | 45.03 | C |
| ATOM | 6273 | CD1 | TYR | B | 14 | 12.346 | 62.368 | 33.443 | 1.00 | 45.11 | C |
| ATOM | 6275 | CE1 | TYR | B | 14 | 12.314 | 61.184 | 34.204 | 1.00 | 44.50 | C |
| ATOM | 6277 | CZ | TYR | B | 14 | 11.978 | 61.258 | 35.556 | 1.00 | 45.22 | C |
| ATOM | 6278 | OH | TYR | B | 14 | 11.908 | 60.155 | 36.388 | 1.00 | 45.51 | O |
| ATOM | 6280 | CE2 | TYR | B | 14 | 11.702 | 62.446 | 36.117 | 1.00 | 37.21 | C |
| ATOM | 6282 | CD2 | TYR | B | 14 | 11.741 | 63.566 | 35.378 | 1.00 | 43.77 | C |
| ATOM | 6284 | C | TYR | B | 14 | 10.776 | 66.739 | 32.217 | 1.00 | 49.55 | C |
| ATOM | 6285 | O | TYR | B | 14 | 11.293 | 66.888 | 31.111 | 1.00 | 52.45 | O |
| ATOM | 6286 | N | TYR | B | 15 | 10.286 | 67.759 | 32.924 | 1.00 | 45.83 | N |
| ATOM | 6288 | CA | TYR | B | 15 | 10.233 | 69.079 | 32.339 | 1.00 | 39.66 | C |
| ATOM | 6290 | CB | TYR | B | 15 | 8.817 | 69.345 | 31.844 | 1.00 | 39.42 | C |
| ATOM | 6293 | CG | TYR | B | 15 | 7.835 | 69.394 | 32.978 | 1.00 | 40.57 | C |
| ATOM | 6294 | CD1 | TYR | B | 15 | 7.743 | 70.510 | 33.798 | 1.00 | 43.59 | C |
| ATOM | 6296 | CE1 | TYR | B | 15 | 6.869 | 70.561 | 34.824 | 1.00 | 34.88 | C |
| ATOM | 6298 | CZ | TYR | B | 15 | 6.065 | 69.496 | 35.062 | 1.00 | 37.24 | C |
| ATOM | 6299 | OH | TYR | B | 15 | 5.168 | 69.511 | 36.110 | 1.00 | 47.03 | O |
| ATOM | 6301 | CE2 | TYR | B | 15 | 6.130 | 68.390 | 34.273 | 1.00 | 41.16 | C |
| ATOM | 6303 | CD2 | TYR | B | 15 | 7.008 | 68.340 | 33.240 | 1.00 | 42.00 | C |
| ATOM | 6305 | C | TYR | B | 15 | 10.686 | 70.194 | 33.276 | 1.00 | 40.31 | C |
| ATOM | 6306 | O | TYR | B | 15 | 10.679 | 70.062 | 34.478 | 1.00 | 37.96 | O |
| ATOM | 6307 | N | VAL | B | 16 | 11.069 | 71.312 | 32.676 | 1.00 | 43.44 | N |
| ATOM | 6309 | CA | VAL | B | 16 | 11.673 | 72.427 | 33.369 | 1.00 | 42.50 | C |
| ATOM | 6311 | CB | VAL | B | 16 | 13.141 | 72.669 | 32.925 | 1.00 | 40.97 | C |
| ATOM | 6313 | CG1 | VAL | B | 16 | 13.240 | 73.090 | 31.465 | 1.00 | 36.56 | C |
| ATOM | 6317 | CG2 | VAL | B | 16 | 13.724 | 73.752 | 33.699 | 1.00 | 45.84 | C |
| ATOM | 6321 | C | VAL | B | 16 | 10.836 | 73.605 | 32.964 | 1.00 | 46.17 | C |
| ATOM | 6322 | O | VAL | B | 16 | 10.405 | 73.700 | 31.818 | 1.00 | 44.78 | O |
| ATOM | 6323 | N | GLU | B | 17 | 10.609 | 74.497 | 33.910 | 1.00 | 47.27 | N |
| ATOM | 6325 | CA | GLU | B | 17 | 9.788 | 75.649 | 33.701 | 1.00 | 48.45 | C |
| ATOM | 6327 | CB | GLU | B | 17 | 9.314 | 76.118 | 35.056 | 1.00 | 50.22 | C |
| ATOM | 6330 | CG | GLU | B | 17 | 8.748 | 77.518 | 35.050 | 1.00 | 49.63 | C |
| ATOM | 6333 | CD | GLU | B | 17 | 8.451 | 78.008 | 36.443 | 1.00 | 52.98 | C |
| ATOM | 6334 | OE1 | GLU | B | 17 | 7.420 | 77.598 | 37.020 | 1.00 | 56.17 | O |
| ATOM | 6335 | OE2 | GLU | B | 17 | 9.251 | 78.812 | 36.957 | 1.00 | 62.26 | O |
| ATOM | 6336 | C | GLU | B | 17 | 10.587 | 76.771 | 33.070 | 1.00 | 49.62 | C |
| ATOM | 6337 | O | GLU | B | 17 | 11.605 | 77.156 | 33.603 | 1.00 | 58.28 | O |
| ATOM | 6338 | N | MET | B | 18 | 10.130 | 77.308 | 31.949 | 1.00 | 52.00 | N |
| ATOM | 6340 | CA | MET | B | 18 | 10.821 | 78.430 | 31.301 | 1.00 | 52.60 | C |
| ATOM | 6342 | CB | MET | B | 18 | 11.435 | 77.960 | 29.976 | 1.00 | 52.70 | C |
| ATOM | 6345 | CG | MET | B | 18 | 12.598 | 77.016 | 30.128 | 1.00 | 55.57 | C |
| ATOM | 6348 | SD | MET | B | 18 | 13.010 | 76.172 | 28.583 | 1.00 | 59.46 | S |
| ATOM | 6349 | CE | MET | B | 18 | 14.275 | 77.236 | 27.930 | 1.00 | 52.14 | C |
| ATOM | 6353 | C | MET | B | 18 | 9.874 | 79.625 | 31.035 | 1.00 | 50.66 | C |
| ATOM | 6354 | O | MET | B | 18 | 8.647 | 79.496 | 31.067 | 1.00 | 49.38 | O |
| ATOM | 6355 | N | THR | B | 19 | 10.452 | 80.786 | 30.780 | 1.00 | 49.10 | N |
| ATOM | 6357 | CA | THR | B | 19 | 9.650 | 81.918 | 30.382 | 1.00 | 51.16 | C |
| ATOM | 6359 | CB | THR | B | 19 | 9.716 | 83.092 | 31.355 | 1.00 | 50.92 | C |
| ATOM | 6361 | OG1 | THR | B | 19 | 10.956 | 83.832 | 31.217 | 1.00 | 46.48 | O |
| ATOM | 6363 | CG2 | THR | B | 19 | 9.658 | 82.591 | 32.767 | 1.00 | 52.58 | C |
| ATOM | 6367 | C | THR | B | 19 | 10.084 | 82.426 | 29.041 | 1.00 | 55.56 | C |
| ATOM | 6368 | O | THR | B | 19 | 11.269 | 82.597 | 28.768 | 1.00 | 55.44 | O |
| ATOM | 6369 | N | VAL | B | 20 | 9.090 | 82.685 | 28.214 | 1.00 | 57.24 | N |
| ATOM | 6371 | CA | VAL | B | 20 | 9.307 | 83.161 | 26.880 | 1.00 | 54.55 | C |
| ATOM | 6373 | CB | VAL | B | 20 | 8.940 | 82.067 | 25.827 | 1.00 | 54.27 | C |
| ATOM | 6375 | CG1 | VAL | B | 20 | 9.851 | 80.829 | 25.982 | 1.00 | 54.44 | C |
| ATOM | 6379 | CG2 | VAL | B | 20 | 7.504 | 81.630 | 25.953 | 1.00 | 55.00 | C |
| ATOM | 6383 | C | VAL | B | 20 | 8.377 | 84.325 | 26.771 | 1.00 | 55.62 | C |
| ATOM | 6384 | O | VAL | B | 20 | 7.234 | 84.209 | 27.224 | 1.00 | 55.85 | O |
| ATOM | 6385 | N | GLY | B | 21 | 8.875 | 85.444 | 26.223 | 1.00 | 56.40 | N |
| ATOM | 6387 | CA | GLY | B | 21 | 8.066 | 86.620 | 25.916 | 1.00 | 54.12 | C |
| ATOM | 6390 | C | GLY | B | 21 | 8.292 | 87.912 | 26.694 | 1.00 | 52.80 | C |
| ATOM | 6391 | O | GLY | B | 21 | 8.913 | 87.910 | 27.737 | 1.00 | 45.49 | O |
| ATOM | 6392 | N | SER | B | 22 | 7.772 | 89.020 | 26.147 | 1.00 | 52.16 | N |
| ATOM | 6394 | CA | SER | B | 22 | 7.729 | 90.308 | 26.811 | 1.00 | 45.94 | C |
| ATOM | 6396 | CB | SER | B | 22 | 8.740 | 91.218 | 26.200 | 1.00 | 48.74 | C |
| ATOM | 6399 | OG | SER | B | 22 | 10.028 | 90.606 | 26.262 | 1.00 | 58.03 | O |
| ATOM | 6401 | C | SER | B | 22 | 6.328 | 90.918 | 26.679 | 1.00 | 51.64 | C |
| ATOM | 6402 | O | SER | B | 22 | 5.916 | 91.373 | 25.610 | 1.00 | 48.63 | O |
| ATOM | 6403 | N | PRO | B | 23 | 5.587 | 90.895 | 27.784 | 1.00 | 51.99 | N |
| ATOM | 6404 | CA | PRO | B | 23 | 6.088 | 90.276 | 29.019 | 1.00 | 51.30 | C |
| ATOM | 6406 | CB | PRO | B | 23 | 4.902 | 90.437 | 29.964 | 1.00 | 54.52 | C |
| ATOM | 6409 | CG | PRO | B | 23 | 3.759 | 90.424 | 29.043 | 1.00 | 50.06 | C |
| ATOM | 6412 | CD | PRO | B | 23 | 4.226 | 91.436 | 27.979 | 1.00 | 49.63 | C |
| ATOM | 6415 | C | PRO | B | 23 | 6.368 | 88.800 | 28.881 | 1.00 | 47.00 | C |
| ATOM | 6416 | O | PRO | B | 23 | 5.860 | 88.229 | 27.944 | 1.00 | 48.90 | O |
| ATOM | 6417 | N | PRO | B | 24 | 7.169 | 88.249 | 29.794 | 1.00 | 53.61 | N |
| ATOM | 6418 | CA | PRO | B | 24 | 7.415 | 86.787 | 30.020 | 1.00 | 54.15 | C |
| ATOM | 6420 | CB | PRO | B | 24 | 8.155 | 86.804 | 31.345 | 1.00 | 51.32 | C |
| ATOM | 6423 | CG | PRO | B | 24 | 8.902 | 88.094 | 31.318 | 1.00 | 50.72 | C |
| ATOM | 6426 | CD | PRO | B | 24 | 7.995 | 89.088 | 30.680 | 1.00 | 53.02 | C |
| ATOM | 6429 | C | PRO | B | 24 | 6.260 | 85.781 | 30.261 | 1.00 | 55.63 | C |
| ATOM | 6430 | O | PRO | B | 24 | 5.567 | 85.900 | 31.270 | 1.00 | 54.27 | O |
| ATOM | 6431 | N | GLN | B | 25 | 6.097 | 84.775 | 29.392 | 1.00 | 57.58 | N |
| ATOM | 6433 | CA | GLN | B | 25 | 5.025 | 83.775 | 29.524 | 1.00 | 56.58 | C |
| ATOM | 6435 | CB | GLN | B | 25 | 4.404 | 83.453 | 28.153 | 1.00 | 58.02 | C |
| ATOM | 6438 | CG | GLN | B | 25 | 3.196 | 84.297 | 27.785 | 1.00 | 61.35 | C |
| ATOM | 6441 | CD | GLN | B | 25 | 2.729 | 84.086 | 26.353 | 1.00 | 63.09 | C |
| ATOM | 6442 | OE1 | GLN | B | 25 | 2.293 | 82.981 | 25.990 | 1.00 | 69.50 | O |
| ATOM | 6443 | NE2 | GLN | B | 25 | 2.802 | 85.135 | 25.543 | 1.00 | 54.51 | N |
| ATOM | 6446 | C | GLN | B | 25 | 5.604 | 82.496 | 30.116 | 1.00 | 58.86 | C |
| ATOM | 6447 | O | GLN | B | 25 | 6.574 | 81.987 | 29.559 | 1.00 | 57.53 | O |
| ATOM | 6448 | N | THR | B | 26 | 5.036 | 82.000 | 31.228 | 1.00 | 52.25 | N |
| ATOM | 6450 | CA | THR | B | 26 | 5.497 | 80.785 | 31.866 | 1.00 | 49.89 | C |
| ATOM | 6452 | CB | THR | B | 26 | 5.043 | 80.761 | 33.304 | 1.00 | 51.53 | C |
| ATOM | 6454 | OG1 | THR | B | 26 | 5.882 | 81.592 | 34.127 | 1.00 | 48.51 | O |
| ATOM | 6456 | CG2 | THR | B | 26 | 5.233 | 79.353 | 33.886 | 1.00 | 49.37 | C |
| ATOM | 6460 | C | THR | B | 26 | 4.970 | 79.505 | 31.224 | 1.00 | 51.74 | C |
| ATOM | 6461 | O | THR | B | 26 | 3.772 | 79.321 | 31.123 | 1.00 | 55.36 | O |
| ATOM | 6462 | N | LEU | B | 27 | 5.861 | 78.603 | 30.808 | 1.00 | 52.47 | N |
| ATOM | 6464 | CA | LEU | B | 27 | 5.440 | 77.291 | 30.296 | 1.00 | 52.98 | C |
| ATOM | 6466 | CB | LEU | B | 27 | 5.447 | 77.292 | 28.768 | 1.00 | 53.80 | C |
| ATOM | 6469 | CG | LEU | B | 27 | 4.766 | 78.485 | 28.088 | 1.00 | 55.67 | C |
| ATOM | 6471 | CD1 | LEU | B | 27 | 5.163 | 78.522 | 26.640 | 1.00 | 52.91 | C |
| ATOM | 6475 | CD2 | LEU | B | 27 | 3.248 | 78.447 | 28.216 | 1.00 | 52.46 | C |
| ATOM | 6479 | C | LEU | B | 27 | 6.344 | 76.152 | 30.825 | 1.00 | 54.38 | C |
| ATOM | 6480 | O | LEU | B | 27 | 7.492 | 76.415 | 31.190 | 1.00 | 55.85 | O |
| ATOM | 6481 | N | ASN | B | 28 | 5.809 | 74.922 | 30.863 | 1.00 | 51.66 | N |
| ATOM | 6483 | CA | ASN | B | 28 | 6.518 | 73.711 | 31.292 | 1.00 | 55.85 | C |
| ATOM | 6485 | CB | ASN | B | 28 | 5.526 | 72.700 | 31.940 | 1.00 | 58.80 | C |
| ATOM | 6488 | CG | ASN | B | 28 | 5.260 | 72.950 | 33.449 | 1.00 | 57.89 | C |
| ATOM | 6489 | OD1 | ASN | B | 28 | 4.145 | 72.685 | 33.961 | 1.00 | 43.49 | O |
| ATOM | 6490 | ND2 | ASN | B | 28 | 6.273 | 73.440 | 34.152 | 1.00 | 52.67 | N |
| ATOM | 6493 | C | ASN | B | 28 | 7.119 | 73.035 | 30.037 | 1.00 | 54.00 | C |
| ATOM | 6494 | O | ASN | B | 28 | 6.411 | 72.858 | 29.040 | 1.00 | 55.20 | O |
| ATOM | 6495 | N | ILE | B | 29 | 8.385 | 72.602 | 30.082 | 1.00 | 49.11 | N |
| ATOM | 6497 | CA | ILE | B | 29 | 9.090 | 72.101 | 28.877 | 1.00 | 41.75 | C |
| ATOM | 6499 | CB | ILE | B | 29 | 10.096 | 73.201 | 28.430 | 1.00 | 41.61 | C |
| ATOM | 6501 | CG1 | ILE | B | 29 | 9.403 | 74.521 | 28.502 | 1.00 | 36.87 | C |
| ATOM | 6504 | CD1 | ILE | B | 29 | 8.620 | 74.882 | 27.272 | 1.00 | 35.29 | C |
| ATOM | 6508 | CG2 | ILE | B | 29 | 10.627 | 72.989 | 27.001 | 1.00 | 45.92 | C |
| ATOM | 6512 | C | ILE | B | 29 | 9.834 | 70.738 | 29.020 | 1.00 | 41.21 | C |
| ATOM | 6513 | O | ILE | B | 29 | 10.821 | 70.657 | 29.735 | 1.00 | 40.42 | O |
| ATOM | 6514 | N | LEU | B | 30 | 9.363 | 69.679 | 28.344 | 1.00 | 40.94 | N |
| ATOM | 6516 | CA | LEU | B | 30 | 10.056 | 68.371 | 28.308 | 1.00 | 42.00 | C |
| ATOM | 6518 | CB | LEU | B | 30 | 9.450 | 67.507 | 27.215 | 1.00 | 41.64 | C |
| ATOM | 6521 | CG | LEU | B | 30 | 9.943 | 66.065 | 27.050 | 1.00 | 41.51 | C |
| ATOM | 6523 | CD1 | LEU | B | 30 | 8.966 | 65.054 | 27.427 | 1.00 | 38.45 | C |
| ATOM | 6527 | CD2 | LEU | B | 30 | 10.258 | 65.805 | 25.617 | 1.00 | 51.30 | C |
| ATOM | 6531 | C | LEU | B | 30 | 11.529 | 68.607 | 27.974 | 1.00 | 46.49 | C |
| ATOM | 6532 | O | LEU | B | 30 | 11.849 | 69.519 | 27.220 | 1.00 | 47.11 | O |
| ATOM | 6533 | N | VAL | B | 31 | 12.414 | 67.781 | 28.527 | 1.00 | 46.41 | N |
| ATOM | 6535 | CA | VAL | B | 31 | 13.858 | 67.880 | 28.288 | 1.00 | 47.73 | C |
| ATOM | 6537 | CB | VAL | B | 31 | 14.633 | 67.834 | 29.620 | 1.00 | 48.19 | C |
| ATOM | 6539 | CG1 | VAL | B | 31 | 16.047 | 67.314 | 29.431 | 1.00 | 52.74 | C |
| ATOM | 6543 | CG2 | VAL | B | 31 | 14.661 | 69.187 | 30.226 | 1.00 | 47.82 | C |
| ATOM | 6547 | C | VAL | B | 31 | 14.251 | 66.709 | 27.422 | 1.00 | 49.66 | C |
| ATOM | 6548 | O | VAL | B | 31 | 14.369 | 65.614 | 27.935 | 1.00 | 50.43 | O |
| ATOM | 6549 | N | ASP | B | 32 | 14.488 | 66.937 | 26.122 | 1.00 | 50.95 | N |
| ATOM | 6551 | CA | ASP | B | 32 | 14.599 | 65.825 | 25.165 | 1.00 | 50.66 | C |
| ATOM | 6553 | CB | ASP | B | 32 | 13.547 | 66.011 | 24.085 | 1.00 | 46.26 | C |
| ATOM | 6556 | CG | ASP | B | 32 | 13.417 | 64.802 | 23.209 | 1.00 | 46.83 | C |
| ATOM | 6557 | OD1 | ASP | B | 32 | 12.601 | 63.952 | 23.614 | 1.00 | 57.65 | O |
| ATOM | 6558 | OD2 | ASP | B | 32 | 14.037 | 64.597 | 22.118 | 1.00 | 28.18 | O |
| ATOM | 6559 | C | ASP | B | 32 | 15.937 | 65.627 | 24.483 | 1.00 | 50.93 | C |
| ATOM | 6560 | O | ASP | B | 32 | 16.137 | 66.095 | 23.372 | 1.00 | 56.78 | O |
| ATOM | 6561 | N | THR | B | 33 | 16.854 | 64.901 | 25.091 | 1.00 | 44.66 | N |
| ATOM | 6563 | CA | THR | B | 33 | 18.135 | 64.814 | 24.467 | 1.00 | 41.11 | C |
| ATOM | 6565 | CB | THR | B | 33 | 19.151 | 64.207 | 25.419 | 1.00 | 44.39 | C |
| ATOM | 6567 | OG1 | THR | B | 33 | 18.649 | 62.989 | 25.966 | 1.00 | 47.50 | O |
| ATOM | 6569 | CG2 | THR | B | 33 | 19.298 | 65.077 | 26.643 | 1.00 | 45.76 | C |
| ATOM | 6573 | C | THR | B | 33 | 18.075 | 64.056 | 23.159 | 1.00 | 43.39 | C |
| ATOM | 6574 | O | THR | B | 33 | 19.101 | 63.882 | 22.521 | 1.00 | 45.31 | O |
| ATOM | 6575 | N | GLY | B | 34 | 16.891 | 63.610 | 22.749 | 1.00 | 43.77 | N |
| ATOM | 6577 | CA | GLY | B | 34 | 16.739 | 62.822 | 21.528 | 1.00 | 46.99 | C |
| ATOM | 6580 | C | GLY | B | 34 | 16.380 | 63.629 | 20.282 | 1.00 | 49.16 | C |
| ATOM | 6581 | O | GLY | B | 34 | 16.324 | 63.093 | 19.151 | 1.00 | 50.21 | O |
| ATOM | 6582 | N | SER | B | 35 | 16.151 | 64.924 | 20.486 | 1.00 | 50.26 | N |
| ATOM | 6584 | CA | SER | B | 35 | 15.776 | 65.838 | 19.394 | 1.00 | 47.28 | C |
| ATOM | 6586 | CB | SER | B | 35 | 14.281 | 66.025 | 19.398 | 1.00 | 43.53 | C |
| ATOM | 6589 | OG | SER | B | 35 | 13.916 | 66.661 | 20.582 | 1.00 | 41.18 | O |
| ATOM | 6591 | C | SER | B | 35 | 16.491 | 67.207 | 19.504 | 1.00 | 43.05 | C |
| ATOM | 6592 | O | SER | B | 35 | 17.273 | 67.446 | 20.440 | 1.00 | 43.12 | O |
| ATOM | 6593 | N | SER | B | 36 | 16.238 | 68.094 | 18.549 | 1.00 | 37.14 | N |
| ATOM | 6595 | CA | SER | B | 36 | 16.982 | 69.332 | 18.512 | 1.00 | 36.80 | C |
| ATOM | 6597 | CB | SER | B | 36 | 18.137 | 69.180 | 17.502 | 1.00 | 40.35 | C |
| ATOM | 6600 | OG | SER | B | 36 | 19.229 | 68.365 | 18.021 | 1.00 | 36.18 | O |
| ATOM | 6602 | C | SER | B | 36 | 16.182 | 70.629 | 18.297 | 1.00 | 42.36 | C |
| ATOM | 6603 | O | SER | B | 36 | 16.739 | 71.718 | 18.339 | 1.00 | 49.70 | O |
| ATOM | 6604 | N | ASN | B | 37 | 14.879 | 70.560 | 18.107 | 1.00 | 45.23 | N |
| ATOM | 6606 | CA | ASN | B | 37 | 14.135 | 71.783 | 17.906 | 1.00 | 44.85 | C |
| ATOM | 6608 | CB | ASN | B | 37 | 13.080 | 71.617 | 16.845 | 1.00 | 43.43 | C |
| ATOM | 6611 | CG | ASN | B | 37 | 13.659 | 71.592 | 15.492 | 1.00 | 49.01 | C |
| ATOM | 6612 | OD1 | ASN | B | 37 | 14.303 | 70.615 | 15.126 | 1.00 | 53.37 | O |
| ATOM | 6613 | ND2 | ASN | B | 37 | 13.450 | 72.678 | 14.715 | 1.00 | 47.23 | N |
| ATOM | 6616 | C | ASN | B | 37 | 13.432 | 72.151 | 19.164 | 1.00 | 43.89 | C |
| ATOM | 6617 | O | ASN | B | 37 | 12.955 | 71.279 | 19.843 | 1.00 | 48.92 | O |
| ATOM | 6618 | N | PHE | B | 38 | 13.379 | 73.447 | 19.454 | 1.00 | 39.62 | N |
| ATOM | 6620 | CA | PHE | B | 38 | 12.666 | 73.984 | 20.588 | 1.00 | 37.09 | C |
| ATOM | 6622 | CB | PHE | B | 38 | 13.518 | 75.084 | 21.189 | 1.00 | 34.12 | C |
| ATOM | 6625 | CG | PHE | B | 38 | 12.915 | 75.809 | 22.291 | 1.00 | 36.88 | C |
| ATOM | 6626 | CD1 | PHE | B | 38 | 13.190 | 77.128 | 22.451 | 1.00 | 43.04 | C |
| ATOM | 6628 | CE1 | PHE | B | 38 | 12.639 | 77.847 | 23.480 | 1.00 | 43.52 | C |
| ATOM | 6630 | CZ | PHE | B | 38 | 11.810 | 77.261 | 24.366 | 1.00 | 45.02 | C |
| ATOM | 6632 | CE2 | PHE | B | 38 | 11.519 | 75.950 | 24.235 | 1.00 | 51.64 | C |
| ATOM | 6634 | CD2 | PHE | B | 38 | 12.082 | 75.213 | 23.185 | 1.00 | 52.30 | C |
| ATOM | 6636 | C | PHE | B | 38 | 11.313 | 74.445 | 20.010 | 1.00 | 40.81 | C |
| ATOM | 6637 | O | PHE | B | 38 | 11.241 | 75.206 | 19.018 | 1.00 | 42.46 | O |
| ATOM | 6638 | N | ALA | B | 39 | 10.238 | 73.940 | 20.607 | 1.00 | 40.42 | N |
| ATOM | 6640 | CA | ALA | B | 39 | 8.903 | 74.198 | 20.105 | 1.00 | 44.90 | C |
| ATOM | 6642 | CB | ALA | B | 39 | 8.473 | 73.061 | 19.113 | 1.00 | 45.36 | C |
| ATOM | 6646 | C | ALA | B | 39 | 7.889 | 74.308 | 21.212 | 1.00 | 41.28 | C |
| ATOM | 6647 | O | ALA | B | 39 | 7.961 | 73.617 | 22.213 | 1.00 | 44.60 | O |
| ATOM | 6648 | N | VAL | B | 40 | 6.929 | 75.177 | 21.046 | 1.00 | 40.49 | N |
| ATOM | 6650 | CA | VAL | B | 40 | 5.885 | 75.186 | 22.027 | 1.00 | 49.83 | C |
| ATOM | 6652 | CB | VAL | B | 40 | 6.020 | 76.354 | 22.997 | 1.00 | 49.55 | C |
| ATOM | 6654 | CG1 | VAL | B | 40 | 7.345 | 76.332 | 23.631 | 1.00 | 46.17 | C |
| ATOM | 6658 | CG2 | VAL | B | 40 | 5.830 | 77.657 | 22.280 | 1.00 | 54.69 | C |
| ATOM | 6662 | C | VAL | B | 40 | 4.496 | 75.202 | 21.404 | 1.00 | 51.60 | C |
| ATOM | 6663 | O | VAL | B | 40 | 4.209 | 75.904 | 20.435 | 1.00 | 56.20 | O |
| ATOM | 6664 | N | GLY | B | 41 | 3.629 | 74.394 | 21.958 | 1.00 | 50.81 | N |
| ATOM | 6666 | CA | GLY | B | 41 | 2.235 | 74.528 | 21.608 | 1.00 | 54.35 | C |
| ATOM | 6669 | C | GLY | B | 41 | 1.861 | 76.000 | 21.658 | 1.00 | 54.27 | C |
| ATOM | 6670 | O | GLY | B | 41 | 2.139 | 76.715 | 22.646 | 1.00 | 50.40 | O |
| ATOM | 6671 | N | ALA | B | 42 | 1.208 | 76.439 | 20.585 | 1.00 | 55.45 | N |
| ATOM | 6673 | CA | ALA | B | 42 | 0.864 | 77.840 | 20.394 | 1.00 | 57.72 | C |
| ATOM | 6675 | CB | ALA | B | 42 | 1.661 | 78.375 | 19.208 | 1.00 | 61.49 | C |
| ATOM | 6679 | C | ALA | B | 42 | −0.620 | 78.089 | 20.158 | 1.00 | 59.93 | C |
| ATOM | 6680 | O | ALA | B | 42 | −1.041 | 79.239 | 19.956 | 1.00 | 54.51 | O |
| ATOM | 6681 | N | ALA | B | 43 | −1.422 | 77.028 | 20.205 | 1.00 | 59.25 | N |
| ATOM | 6683 | CA | ALA | B | 43 | −2.838 | 77.148 | 19.904 | 1.00 | 58.08 | C |
| ATOM | 6685 | CB | ALA | B | 43 | −3.069 | 76.951 | 18.403 | 1.00 | 57.09 | C |
| ATOM | 6689 | C | ALA | B | 43 | −3.577 | 76.084 | 20.669 | 1.00 | 57.28 | C |
| ATOM | 6690 | O | ALA | B | 43 | −3.047 | 75.011 | 20.869 | 1.00 | 57.71 | O |
| ATOM | 6691 | N | PRO | B | 44 | −4.809 | 76.358 | 21.067 | 1.00 | 58.13 | N |
| ATOM | 6692 | CA | PRO | B | 44 | −5.568 | 75.407 | 21.856 | 1.00 | 55.97 | C |
| ATOM | 6694 | CB | PRO | B | 44 | −7.009 | 75.819 | 21.550 | 1.00 | 57.92 | C |
| ATOM | 6697 | CG | PRO | B | 44 | −6.845 | 76.827 | 20.407 | 1.00 | 57.30 | C |
| ATOM | 6700 | CD | PRO | B | 44 | −5.628 | 77.554 | 20.804 | 1.00 | 57.17 | C |
| ATOM | 6703 | C | PRO | B | 44 | −5.339 | 74.012 | 21.388 | 1.00 | 55.26 | C |
| ATOM | 6704 | O | PRO | B | 44 | −5.058 | 73.798 | 20.199 | 1.00 | 55.54 | O |
| ATOM | 6705 | N | HIS | B | 45 | −5.458 | 73.083 | 22.330 | 1.00 | 51.68 | N |
| ATOM | 6707 | CA | HIS | B | 45 | −5.380 | 71.662 | 22.074 | 1.00 | 49.63 | C |
| ATOM | 6709 | CB | HIS | B | 45 | −3.960 | 71.199 | 21.920 | 1.00 | 47.73 | C |
| ATOM | 6712 | CG | HIS | B | 45 | −3.854 | 69.743 | 21.624 | 1.00 | 47.10 | C |
| ATOM | 6713 | ND1 | HIS | B | 45 | −3.661 | 68.805 | 22.613 | 1.00 | 47.83 | N |
| ATOM | 6715 | CE1 | HIS | B | 45 | −3.628 | 67.602 | 22.062 | 1.00 | 48.34 | C |
| ATOM | 6717 | NE2 | HIS | B | 45 | −3.798 | 67.728 | 20.755 | 1.00 | 39.59 | N |
| ATOM | 6719 | CD2 | HIS | B | 45 | −3.932 | 69.058 | 20.454 | 1.00 | 37.38 | C |
| ATOM | 6721 | C | HIS | B | 45 | −5.990 | 71.052 | 23.311 | 1.00 | 54.88 | C |
| ATOM | 6722 | O | HIS | B | 45 | −5.850 | 71.599 | 24.387 | 1.00 | 61.00 | O |
| ATOM | 6723 | N | PRO | B | 46 | −6.670 | 69.930 | 23.205 | 1.00 | 58.61 | N |
| ATOM | 6724 | CA | PRO | B | 46 | −7.391 | 69.427 | 24.372 | 1.00 | 58.25 | C |
| ATOM | 6726 | CB | PRO | B | 46 | −8.093 | 68.157 | 23.840 | 1.00 | 56.69 | C |
| ATOM | 6729 | CG | PRO | B | 46 | −8.000 | 68.181 | 22.353 | 1.00 | 55.11 | C |
| ATOM | 6732 | CD | PRO | B | 46 | −6.815 | 69.046 | 22.032 | 1.00 | 60.22 | C |
| ATOM | 6735 | C | PRO | B | 46 | −6.491 | 69.075 | 25.575 | 1.00 | 57.99 | C |
| ATOM | 6736 | O | PRO | B | 46 | −6.953 | 68.992 | 26.716 | 1.00 | 56.13 | O |
| ATOM | 6737 | N | PHE | B | 47 | −5.206 | 68.871 | 25.356 | 1.00 | 55.88 | N |
| ATOM | 6739 | CA | PHE | B | 47 | −4.396 | 68.376 | 26.470 | 1.00 | 51.81 | C |
| ATOM | 6741 | CB | PHE | B | 47 | −3.499 | 67.274 | 25.982 | 1.00 | 44.88 | C |
| ATOM | 6744 | CG | PHE | B | 47 | −4.234 | 66.044 | 25.604 | 1.00 | 46.77 | C |
| ATOM | 6745 | CD1 | PHE | B | 47 | −3.640 | 65.093 | 24.782 | 1.00 | 49.70 | C |
| ATOM | 6747 | CE1 | PHE | B | 47 | −4.315 | 63.949 | 24.429 | 1.00 | 48.35 | C |
| ATOM | 6749 | CZ | PHE | B | 47 | −5.596 | 63.746 | 24.901 | 1.00 | 43.45 | C |
| ATOM | 6751 | CE2 | PHE | B | 47 | −6.205 | 64.682 | 25.729 | 1.00 | 36.92 | C |
| ATOM | 6753 | CD2 | PHE | B | 47 | −5.530 | 65.822 | 26.070 | 1.00 | 43.75 | C |
| ATOM | 6755 | C | PHE | B | 47 | −3.532 | 69.385 | 27.152 | 1.00 | 53.07 | C |
| ATOM | 6756 | O | PHE | B | 47 | −2.763 | 69.015 | 28.046 | 1.00 | 56.38 | O |
| ATOM | 6757 | N | LEU | B | 48 | −3.661 | 70.647 | 26.760 | 1.00 | 48.06 | N |
| ATOM | 6759 | CA | LEU | B | 48 | −2.719 | 71.620 | 27.207 | 1.00 | 49.14 | C |
| ATOM | 6761 | CB | LEU | B | 48 | −2.107 | 72.333 | 26.014 | 1.00 | 50.40 | C |
| ATOM | 6764 | CG | LEU | B | 48 | −1.214 | 71.667 | 24.990 | 1.00 | 47.31 | C |
| ATOM | 6766 | CD1 | LEU | B | 48 | −1.252 | 72.578 | 23.755 | 1.00 | 47.58 | C |
| ATOM | 6770 | CD2 | LEU | B | 48 | −0.179 | 71.567 | 25.528 | 1.00 | 48.26 | C |
| ATOM | 6774 | C | LEU | B | 48 | −3.453 | 72.636 | 27.955 | 1.00 | 53.07 | C |
| ATOM | 6775 | O | LEU | B | 48 | −4.383 | 73.194 | 27.396 | 1.00 | 55.21 | O |
| ATOM | 6776 | N | HIS | B | 49 | −3.033 | 72.940 | 29.181 | 1.00 | 57.93 | N |
| ATOM | 6778 | CA | HIS | B | 49 | −3.757 | 73.897 | 30.015 | 1.00 | 56.76 | C |
| ATOM | 6780 | CB | HIS | B | 49 | −3.451 | 73.706 | 31.497 | 1.00 | 61.80 | C |
| ATOM | 6783 | CG | HIS | B | 49 | −4.070 | 72.466 | 32.048 | 1.00 | 67.51 | C |
| ATOM | 6784 | ND1 | HIS | B | 49 | −5.274 | 71.984 | 31.583 | 1.00 | 65.77 | N |
| ATOM | 6786 | CE1 | HIS | B | 49 | −5.579 | 70.872 | 32.226 | 1.00 | 68.88 | C |
| ATOM | 6788 | NE2 | HIS | B | 49 | −4.615 | 70.615 | 33.092 | 1.00 | 72.14 | N |
| ATOM | 6790 | CD2 | HIS | B | 49 | −3.654 | 71.596 | 32.999 | 1.00 | 70.39 | C |
| ATOM | 6792 | C | HIS | B | 49 | −3.484 | 75.276 | 29.617 | 1.00 | 55.32 | C |
| ATOM | 6793 | O | HIS | B | 49 | −4.365 | 76.100 | 29.747 | 1.00 | 54.19 | O |
| ATOM | 6794 | N | ARG | B | 50 | −2.278 | 75.563 | 29.139 | 1.00 | 57.08 | N |
| ATOM | 6796 | CA | ARG | B | 50 | −1.920 | 76.933 | 28.702 | 1.00 | 52.78 | C |
| ATOM | 6798 | CB | ARG | B | 50 | −1.133 | 77.660 | 29.796 | 1.00 | 48.76 | C |
| ATOM | 6801 | CG | ARG | B | 50 | −0.218 | 76.713 | 30.591 | 1.00 | 53.36 | C |
| ATOM | 6804 | CD | ARG | B | 50 | 0.743 | 77.384 | 31.593 | 1.00 | 54.71 | C |
| ATOM | 6807 | NE | ARG | B | 50 | 1.853 | 76.474 | 31.868 | 1.00 | 61.19 | N |
| ATOM | 6809 | CZ | ARG | B | 50 | 2.737 | 76.603 | 32.838 | 1.00 | 59.08 | C |
| ATOM | 6810 | NH1 | ARG | B | 50 | 2.693 | 77.609 | 33.688 | 1.00 | 60.26 | N |
| ATOM | 6813 | NH2 | ARG | B | 50 | 3.681 | 75.703 | 32.952 | 1.00 | 66.01 | N |
| ATOM | 6816 | C | ARG | B | 50 | −1.078 | 76.790 | 27.438 | 1.00 | 54.68 | C |
| ATOM | 6817 | O | ARG | B | 50 | −0.699 | 75.669 | 27.081 | 1.00 | 57.99 | O |
| ATOM | 6818 | N | TYR | B | 51 | −0.791 | 77.896 | 26.746 | 1.00 | 55.14 | N |
| ATOM | 6820 | CA | TYR | B | 51 | 0.040 | 77.836 | 25.534 | 1.00 | 51.39 | C |
| ATOM | 6822 | CB | TYR | B | 51 | −0.692 | 77.265 | 24.296 | 1.00 | 52.11 | C |
| ATOM | 6825 | CG | TYR | B | 51 | −1.939 | 77.986 | 23.819 | 1.00 | 50.59 | C |
| ATOM | 6826 | CD1 | TYR | B | 51 | −1.872 | 79.145 | 23.080 | 1.00 | 53.23 | C |
| ATOM | 6828 | CE1 | TYR | B | 51 | −3.007 | 79.776 | 22.659 | 1.00 | 54.06 | C |
| ATOM | 6830 | CZ | TYR | B | 51 | −4.238 | 79.243 | 22.977 | 1.00 | 56.68 | C |
| ATOM | 6831 | OH | TYR | B | 51 | −5.428 | 79.833 | 22.574 | 1.00 | 55.76 | O |
| ATOM | 6833 | CE2 | TYR | B | 51 | −4.311 | 78.099 | 23.699 | 1.00 | 56.57 | C |
| ATOM | 6835 | CD2 | TYR | B | 51 | −3.178 | 77.485 | 24.107 | 1.00 | 55.59 | C |
| ATOM | 6837 | C | TYR | B | 51 | 0.654 | 79.155 | 25.198 | 1.00 | 49.19 | C |
| ATOM | 6838 | O | TYR | B | 51 | 0.394 | 80.160 | 25.825 | 1.00 | 48.94 | O |
| ATOM | 6839 | N | TYR | B | 52 | 1.503 | 79.151 | 24.193 | 1.00 | 47.59 | N |
| ATOM | 6841 | CA | TYR | B | 52 | 2.182 | 80.378 | 23.852 | 1.00 | 50.52 | C |
| ATOM | 6843 | CB | TYR | B | 52 | 3.496 | 80.019 | 23.199 | 1.00 | 44.85 | C |
| ATOM | 6846 | CG | TYR | B | 52 | 4.382 | 81.161 | 22.853 | 1.00 | 43.08 | C |
| ATOM | 6847 | CD1 | TYR | B | 52 | 4.498 | 82.273 | 23.656 | 1.00 | 45.53 | C |
| ATOM | 6849 | CE1 | TYR | B | 52 | 5.355 | 83.314 | 23.297 | 1.00 | 43.96 | C |
| ATOM | 6851 | CZ | TYR | B | 52 | 6.080 | 83.202 | 22.134 | 1.00 | 41.86 | C |
| ATOM | 6852 | OH | TYR | B | 52 | 6.952 | 84.165 | 21.695 | 1.00 | 54.44 | O |
| ATOM | 6854 | CE2 | TYR | B | 52 | 5.969 | 82.123 | 21.357 | 1.00 | 43.09 | C |
| ATOM | 6856 | CD2 | TYR | B | 52 | 5.138 | 81.113 | 21.705 | 1.00 | 46.61 | C |
| ATOM | 6858 | C | TYR | B | 52 | 1.327 | 81.241 | 22.914 | 1.00 | 53.39 | C |
| ATOM | 6859 | O | TYR | B | 52 | 0.897 | 80.763 | 21.840 | 1.00 | 55.11 | O |
| ATOM | 6860 | N | GLN | B | 53 | 1.066 | 82.488 | 23.312 | 1.00 | 49.66 | N |
| ATOM | 6862 | CA | GLN | B | 53 | 0.350 | 83.402 | 22.424 | 1.00 | 53.39 | C |
| ATOM | 6864 | CB | GLN | B | 53 | −0.928 | 83.978 | 23.029 | 1.00 | 57.67 | C |
| ATOM | 6867 | CG | GLN | B | 53 | −1.673 | 83.154 | 24.073 | 1.00 | 58.49 | C |
| ATOM | 6870 | CD | GLN | B | 53 | −2.674 | 84.056 | 24.766 | 1.00 | 63.66 | C |
| ATOM | 6871 | OE1 | GLN | B | 53 | −2.341 | 84.707 | 25.784 | 1.00 | 62.18 | O |
| ATOM | 6872 | NE2 | GLN | B | 53 | −3.891 | 84.146 | 24.200 | 1.00 | 60.51 | N |
| ATOM | 6875 | C | GLN | B | 53 | 1.254 | 84.569 | 21.988 | 1.00 | 54.38 | C |
| ATOM | 6876 | O | GLN | B | 53 | 1.569 | 85.510 | 22.750 | 1.00 | 49.25 | O |
| ATOM | 6877 | N | ARG | B | 54 | 1.657 | 84.494 | 20.735 | 1.00 | 50.22 | N |
| ATOM | 6879 | CA | ARG | B | 54 | 2.547 | 85.458 | 20.202 | 1.00 | 45.95 | C |
| ATOM | 6881 | CB | ARG | B | 54 | 2.907 | 85.045 | 18.778 | 1.00 | 45.10 | C |
| ATOM | 6884 | CG | ARG | B | 54 | 3.655 | 83.672 | 18.783 | 1.00 | 39.53 | C |
| ATOM | 6887 | CD | ARG | B | 54 | 3.679 | 82.890 | 17.474 | 1.00 | 42.75 | C |
| ATOM | 6890 | NE | ARG | B | 54 | 2.387 | 82.353 | 17.079 | 1.00 | 47.37 | N |
| ATOM | 6892 | CZ | ARG | B | 54 | 2.139 | 81.788 | 15.910 | 1.00 | 45.51 | C |
| ATOM | 6893 | NH1 | ARG | B | 54 | 3.100 | 81.680 | 15.024 | 1.00 | 51.53 | N |
| ATOM | 6896 | NH2 | ARG | B | 54 | 0.931 | 81.330 | 15.618 | 1.00 | 42.27 | N |
| ATOM | 6899 | C | ARG | B | 54 | 1.939 | 86.836 | 20.386 | 1.00 | 52.82 | C |
| ATOM | 6900 | O | ARG | B | 54 | 2.439 | 87.560 | 21.225 | 1.00 | 59.82 | O |
| ATOM | 6901 | N | GLN | B | 55 | 0.855 | 87.196 | 19.686 | 1.00 | 57.85 | N |
| ATOM | 6903 | CA | GLN | B | 55 | 0.303 | 88.572 | 19.736 | 1.00 | 57.16 | C |
| ATOM | 6905 | CB | GLN | B | 55 | −1.194 | 88.640 | 19.347 | 1.00 | 62.44 | C |
| ATOM | 6908 | CG | GLN | B | 55 | −1.469 | 88.563 | 17.810 | 1.00 | 70.53 | C |
| ATOM | 6911 | CD | GLN | B | 55 | −2.978 | 88.687 | 17.382 | 1.00 | 79.43 | C |
| ATOM | 6912 | OE1 | GLN | B | 55 | −3.369 | 89.660 | 16.701 | 1.00 | 80.66 | O |
| ATOM | 6913 | NE2 | GLN | B | 55 | −3.802 | 87.692 | 17.764 | 1.00 | 79.42 | N |
| ATOM | 6916 | C | GLN | B | 55 | 0.533 | 89.312 | 21.058 | 1.00 | 57.92 | C |
| ATOM | 6917 | O | GLN | B | 55 | 0.360 | 90.526 | 21.117 | 1.00 | 56.14 | O |
| ATOM | 6918 | N | LEU | B | 56 | 0.930 | 88.622 | 22.121 | 1.00 | 55.89 | N |
| ATOM | 6920 | CA | LEU | B | 56 | 1.213 | 89.336 | 23.370 | 1.00 | 58.18 | C |
| ATOM | 6922 | CB | LEU | B | 56 | 0.712 | 88.524 | 24.540 | 1.00 | 60.42 | C |
| ATOM | 6925 | CG | LEU | B | 56 | −0.721 | 88.029 | 24.402 | 1.00 | 62.37 | C |
| ATOM | 6927 | CD1 | LEU | B | 56 | −1.296 | 87.699 | 25.797 | 1.00 | 63.41 | C |
| ATOM | 6931 | CD2 | LEU | B | 56 | −1.598 | 89.054 | 23.676 | 1.00 | 62.35 | C |
| ATOM | 6935 | C | LEU | B | 56 | 2.666 | 89.723 | 23.690 | 1.00 | 60.41 | C |
| ATOM | 6936 | O | LEU | B | 56 | 2.861 | 90.367 | 24.719 | 1.00 | 60.04 | O |
| ATOM | 6937 | N | SER | B | 57 | 3.663 | 89.348 | 22.857 | 1.00 | 57.76 | N |
| ATOM | 6939 | CA | SER | B | 57 | 5.071 | 89.701 | 23.116 | 1.00 | 54.64 | C |
| ATOM | 6941 | CB | SER | B | 57 | 5.991 | 88.475 | 23.117 | 1.00 | 56.81 | C |
| ATOM | 6944 | OG | SER | B | 57 | 7.344 | 88.844 | 23.423 | 1.00 | 54.08 | O |
| ATOM | 6946 | C | SER | B | 57 | 5.661 | 90.710 | 22.153 | 1.00 | 53.95 | C |
| ATOM | 6947 | O | SER | B | 57 | 5.658 | 90.533 | 20.942 | 1.00 | 55.46 | O |
| ATOM | 6948 | N | SER | B | 58 | 6.218 | 91.755 | 22.738 | 1.00 | 52.39 | N |
| ATOM | 6950 | CA | SER | B | 58 | 6.758 | 92.873 | 22.011 | 1.00 | 46.14 | C |
| ATOM | 6952 | CB | SER | B | 58 | 6.820 | 94.070 | 22.947 | 1.00 | 47.29 | C |
| ATOM | 6955 | OG | SER | B | 58 | 7.567 | 93.825 | 24.116 | 1.00 | 47.06 | O |
| ATOM | 6957 | C | SER | B | 58 | 8.114 | 92.536 | 21.420 | 1.00 | 47.84 | C |
| ATOM | 6958 | O | SER | B | 58 | 8.576 | 93.177 | 20.478 | 1.00 | 47.48 | O |
| ATOM | 6959 | N | THR | B | 59 | 8.747 | 91.510 | 21.965 | 1.00 | 48.33 | N |
| ATOM | 6961 | CA | THR | B | 59 | 10.006 | 91.008 | 21.429 | 1.00 | 42.92 | C |
| ATOM | 6963 | CB | THR | B | 59 | 10.885 | 90.549 | 22.559 | 1.00 | 42.65 | C |
| ATOM | 6965 | OG1 | THR | B | 59 | 10.128 | 89.795 | 23.523 | 1.00 | 49.24 | O |
| ATOM | 6967 | CG2 | THR | B | 59 | 11.359 | 91.709 | 23.347 | 1.00 | 45.57 | C |
| ATOM | 6971 | C | THR | B | 59 | 9.812 | 89.853 | 20.405 | 1.00 | 45.53 | C |
| ATOM | 6972 | O | THR | B | 59 | 10.800 | 89.319 | 19.883 | 1.00 | 42.19 | O |
| ATOM | 6973 | N | TYR | B | 60 | 8.566 | 89.465 | 20.113 | 1.00 | 44.25 | N |
| ATOM | 6975 | CA | TYR | B | 60 | 8.312 | 88.440 | 19.089 | 1.00 | 42.52 | C |
| ATOM | 6977 | CB | TYR | B | 60 | 6.854 | 88.060 | 19.079 | 1.00 | 42.61 | C |
| ATOM | 6980 | CG | TYR | B | 60 | 6.444 | 87.188 | 17.912 | 1.00 | 39.56 | C |
| ATOM | 6981 | CD1 | TYR | B | 60 | 6.853 | 85.902 | 17.825 | 1.00 | 39.91 | C |
| ATOM | 6983 | CE1 | TYR | B | 60 | 6.478 | 85.088 | 16.766 | 1.00 | 37.80 | C |
| ATOM | 6985 | CZ | TYR | B | 60 | 5.693 | 85.576 | 15.787 | 1.00 | 41.24 | C |
| ATOM | 6986 | OH | TYR | B | 60 | 5.341 | 84.743 | 14.726 | 1.00 | 49.70 | O |
| ATOM | 6988 | CE2 | TYR | B | 60 | 5.263 | 86.875 | 15.855 | 1.00 | 38.20 | C |
| ATOM | 6990 | CD2 | TYR | B | 60 | 5.636 | 87.661 | 16.909 | 1.00 | 40.65 | C |
| ATOM | 6992 | C | TYR | B | 60 | 8.593 | 88.944 | 17.691 | 1.00 | 47.70 | C |
| ATOM | 6993 | O | TYR | B | 60 | 8.100 | 89.986 | 17.310 | 1.00 | 45.23 | O |
| ATOM | 6994 | N | ARG | B | 61 | 9.376 | 88.195 | 16.913 | 1.00 | 53.11 | N |
| ATOM | 6996 | CA | ARG | B | 61 | 9.536 | 88.482 | 15.488 | 1.00 | 47.96 | C |
| ATOM | 6998 | CB | ARG | B | 61 | 10.866 | 89.084 | 15.173 | 1.00 | 48.51 | C |
| ATOM | 7001 | CG | ARG | B | 61 | 11.328 | 90.014 | 16.186 | 1.00 | 47.51 | C |
| ATOM | 7004 | CD | ARG | B | 61 | 12.777 | 90.163 | 16.129 | 1.00 | 46.49 | C |
| ATOM | 7007 | NE | ARG | B | 61 | 13.175 | 91.480 | 16.568 | 1.00 | 53.97 | N |
| ATOM | 7009 | CZ | ARG | B | 61 | 14.425 | 91.808 | 16.746 | 1.00 | 57.00 | C |
| ATOM | 7010 | NH1 | ARG | B | 61 | 15.338 | 90.893 | 16.529 | 1.00 | 62.31 | N |
| ATOM | 7013 | NH2 | ARG | B | 61 | 14.778 | 93.015 | 17.141 | 1.00 | 55.13 | N |
| ATOM | 7016 | C | ARG | B | 61 | 9.346 | 87.181 | 14.730 | 1.00 | 48.27 | C |
| ATOM | 7017 | O | ARG | B | 61 | 9.609 | 86.115 | 15.238 | 1.00 | 52.99 | O |
| ATOM | 7018 | N | ASP | B | 62 | 8.910 | 87.304 | 13.496 | 1.00 | 44.77 | N |
| ATOM | 7020 | CA | ASP | B | 62 | 8.336 | 86.217 | 12.776 | 1.00 | 46.62 | C |
| ATOM | 7022 | CB | ASP | B | 62 | 6.934 | 86.722 | 12.384 | 1.00 | 49.17 | C |
| ATOM | 7025 | CG | ASP | B | 62 | 6.177 | 85.815 | 11.468 | 1.00 | 53.87 | C |
| ATOM | 7026 | OD1 | ASP | B | 62 | 6.754 | 84.805 | 11.004 | 1.00 | 55.89 | O |
| ATOM | 7027 | OD2 | ASP | B | 62 | 4.965 | 86.076 | 11.152 | 1.00 | 54.79 | O |
| ATOM | 7028 | C | ASP | B | 62 | 9.225 | 85.892 | 11.608 | 1.00 | 49.58 | C |
| ATOM | 7029 | O | ASP | B | 62 | 9.620 | 86.763 | 10.849 | 1.00 | 55.62 | O |
| ATOM | 7030 | N | LEU | B | 63 | 9.559 | 84.624 | 11.453 | 1.00 | 45.53 | N |
| ATOM | 7032 | CA | LEU | B | 63 | 10.464 | 84.255 | 10.394 | 1.00 | 42.20 | C |
| ATOM | 7034 | CB | LEU | B | 63 | 11.360 | 83.083 | 10.835 | 1.00 | 41.42 | C |
| ATOM | 7037 | CG | LEU | B | 63 | 12.548 | 83.387 | 11.771 | 1.00 | 34.55 | C |
| ATOM | 7039 | CD1 | LEU | B | 63 | 12.847 | 82.271 | 12.775 | 1.00 | 36.00 | C |
| ATOM | 7043 | CD2 | LEU | B | 63 | 13.742 | 83.634 | 10.972 | 1.00 | 36.18 | C |
| ATOM | 7047 | C | LEU | B | 63 | 9.736 | 83.913 | 9.112 | 1.00 | 45.11 | C |
| ATOM | 7048 | O | LEU | B | 63 | 10.388 | 83.773 | 8.099 | 1.00 | 51.74 | O |
| ATOM | 7049 | N | ARG | B | 64 | 8.407 | 83.778 | 9.132 | 1.00 | 45.09 | N |
| ATOM | 7051 | CA | ARG | B | 64 | 7.627 | 83.437 | 7.918 | 1.00 | 45.81 | C |
| ATOM | 7053 | CB | ARG | B | 64 | 7.621 | 84.590 | 6.932 | 1.00 | 42.03 | C |
| ATOM | 7056 | CG | ARG | B | 64 | 8.317 | 85.837 | 7.448 | 1.00 | 48.52 | C |
| ATOM | 7059 | CD | ARG | B | 64 | 7.647 | 87.151 | 7.097 | 1.00 | 42.44 | C |
| ATOM | 7062 | NE | ARG | B | 64 | 8.116 | 87.704 | 5.832 | 1.00 | 45.11 | N |
| ATOM | 7064 | CZ | ARG | B | 64 | 9.098 | 88.601 | 5.721 | 1.00 | 47.61 | C |
| ATOM | 7065 | NH1 | ARG | B | 64 | 9.754 | 89.057 | 6.786 | 1.00 | 49.17 | N |
| ATOM | 7068 | NH2 | ARG | B | 64 | 9.431 | 89.044 | 4.531 | 1.00 | 47.35 | N |
| ATOM | 7071 | C | ARG | B | 64 | 8.161 | 82.208 | 7.176 | 1.00 | 48.40 | C |
| ATOM | 7072 | O | ARG | B | 64 | 8.654 | 82.321 | 6.041 | 1.00 | 50.59 | O |
| ATOM | 7073 | N | LYS | B | 65 | 8.050 | 81.058 | 7.832 | 1.00 | 44.70 | N |
| ATOM | 7075 | CA | LYS | B | 65 | 8.589 | 79.783 | 7.378 | 1.00 | 42.44 | C |
| ATOM | 7077 | CB | LYS | B | 65 | 10.110 | 79.799 | 7.680 | 1.00 | 42.17 | C |
| ATOM | 7080 | CG | LYS | B | 65 | 11.080 | 78.794 | 6.970 | 1.00 | 46.99 | C |
| ATOM | 7083 | CD | LYS | B | 65 | 12.568 | 78.665 | 7.710 | 1.00 | 41.67 | C |
| ATOM | 7086 | CE | LYS | B | 65 | 13.207 | 80.010 | 8.378 | 1.00 | 31.79 | C |
| ATOM | 7089 | NZ | LYS | B | 65 | 14.690 | 79.980 | 8.772 | 1.00 | 2.00 | N |
| ATOM | 7093 | C | LYS | B | 65 | 7.817 | 78.802 | 8.271 | 1.00 | 40.97 | C |
| ATOM | 7094 | O | LYS | B | 65 | 7.650 | 79.069 | 9.459 | 1.00 | 39.90 | O |
| ATOM | 7095 | N | GLY | B | 66 | 7.299 | 77.707 | 7.711 | 1.00 | 45.11 | N |
| ATOM | 7097 | CA | GLY | B | 66 | 6.632 | 76.656 | 8.487 | 1.00 | 37.19 | C |
| ATOM | 7100 | C | GLY | B | 66 | 7.643 | 75.541 | 8.684 | 1.00 | 43.52 | C |
| ATOM | 7101 | O | GLY | B | 66 | 8.638 | 75.469 | 7.970 | 1.00 | 43.64 | O |
| ATOM | 7102 | N | VAL | B | 67 | 7.398 | 74.674 | 9.652 | 1.00 | 48.15 | N |
| ATOM | 7104 | CA | VAL | B | 67 | 8.300 | 73.575 | 9.969 | 1.00 | 47.91 | C |
| ATOM | 7106 | CB | VAL | B | 67 | 9.347 | 74.093 | 10.970 | 1.00 | 46.06 | C |
| ATOM | 7108 | CG1 | VAL | B | 67 | 8.684 | 74.740 | 12.148 | 1.00 | 53.01 | C |
| ATOM | 7112 | CG2 | VAL | B | 67 | 10.242 | 73.023 | 11.460 | 1.00 | 45.35 | C |
| ATOM | 7116 | C | VAL | B | 67 | 7.531 | 72.375 | 10.546 | 1.00 | 48.93 | C |
| ATOM | 7117 | O | VAL | B | 67 | 6.604 | 72.553 | 11.306 | 1.00 | 50.43 | O |
| ATOM | 7118 | N | TYR | B | 68 | 7.920 | 71.159 | 10.173 | 1.00 | 54.33 | N |
| ATOM | 7120 | CA | TYR | B | 68 | 7.307 | 69.912 | 10.681 | 1.00 | 54.23 | C |
| ATOM | 7122 | CB | TYR | B | 68 | 6.674 | 69.180 | 9.496 | 1.00 | 58.61 | C |
| ATOM | 7125 | CG | TYR | B | 68 | 6.194 | 67.743 | 9.740 | 1.00 | 62.41 | C |
| ATOM | 7126 | CD1 | TYR | B | 68 | 5.151 | 67.465 | 10.624 | 1.00 | 65.98 | C |
| ATOM | 7128 | CE1 | TYR | B | 68 | 4.709 | 66.183 | 10.833 | 1.00 | 65.58 | C |
| ATOM | 7130 | CZ | TYR | B | 68 | 5.303 | 65.145 | 10.156 | 1.00 | 70.65 | C |
| ATOM | 7131 | OH | TYR | B | 68 | 4.874 | 63.855 | 10.350 | 1.00 | 76.70 | O |
| ATOM | 7133 | CE2 | TYR | B | 68 | 6.330 | 65.379 | 9.274 | 1.00 | 68.48 | C |
| ATOM | 7135 | CD2 | TYR | B | 68 | 6.766 | 66.677 | 9.067 | 1.00 | 68.01 | C |
| ATOM | 7137 | C | TYR | B | 68 | 8.354 | 68.969 | 11.289 | 1.00 | 52.15 | C |
| ATOM | 7138 | O | TYR | B | 68 | 9.446 | 68.840 | 10.735 | 1.00 | 51.11 | O |
| ATOM | 7139 | N | VAL | B | 69 | 8.028 | 68.308 | 12.407 | 1.00 | 48.81 | N |
| ATOM | 7141 | CA | VAL | B | 69 | 8.931 | 67.350 | 13.043 | 1.00 | 41.24 | C |
| ATOM | 7143 | CB | VAL | B | 69 | 9.433 | 67.888 | 14.381 | 1.00 | 46.72 | C |
| ATOM | 7145 | CG1 | VAL | B | 69 | 10.525 | 66.959 | 14.976 | 1.00 | 50.12 | C |
| ATOM | 7149 | CG2 | VAL | B | 69 | 9.951 | 69.297 | 14.276 | 1.00 | 44.49 | C |
| ATOM | 7153 | C | VAL | B | 69 | 8.254 | 66.073 | 13.454 | 1.00 | 42.06 | C |
| ATOM | 7154 | O | VAL | B | 69 | 7.427 | 66.130 | 14.335 | 1.00 | 49.53 | O |
| ATOM | 7155 | N | PRO | B | 70 | 8.562 | 64.923 | 12.851 | 1.00 | 44.13 | N |
| ATOM | 7156 | CA | PRO | B | 70 | 8.124 | 63.637 | 13.369 | 1.00 | 40.92 | C |
| ATOM | 7158 | CB | PRO | B | 70 | 8.103 | 62.804 | 12.106 | 1.00 | 42.89 | C |
| ATOM | 7161 | CG | PRO | B | 70 | 9.343 | 63.146 | 11.506 | 1.00 | 41.09 | C |
| ATOM | 7164 | CD | PRO | B | 70 | 9.306 | 64.697 | 11.594 | 1.00 | 47.61 | C |
| ATOM | 7167 | C | PRO | B | 70 | 9.149 | 62.940 | 14.268 | 1.00 | 49.11 | C |
| ATOM | 7168 | O | PRO | B | 70 | 10.376 | 62.995 | 14.009 | 1.00 | 49.67 | O |
| ATOM | 7169 | N | TYR | B | 71 | 8.645 | 62.251 | 15.290 | 1.00 | 50.47 | N |
| ATOM | 7171 | CA | TYR | B | 71 | 9.476 | 61.426 | 16.149 | 1.00 | 53.44 | C |
| ATOM | 7173 | CB | TYR | B | 71 | 9.170 | 61.786 | 17.587 | 1.00 | 53.95 | C |
| ATOM | 7176 | CG | TYR | B | 71 | 9.281 | 63.268 | 17.848 | 1.00 | 54.07 | C |
| ATOM | 7177 | CD1 | TYR | B | 71 | 10.380 | 63.799 | 18.509 | 1.00 | 54.07 | C |
| ATOM | 7179 | CE1 | TYR | B | 71 | 10.474 | 65.154 | 18.746 | 1.00 | 54.76 | C |
| ATOM | 7181 | CZ | TYR | B | 71 | 9.475 | 65.989 | 18.322 | 1.00 | 49.10 | C |
| ATOM | 7182 | OH | TYR | B | 71 | 9.564 | 67.326 | 18.547 | 1.00 | 49.20 | O |
| ATOM | 7184 | CE2 | TYR | B | 71 | 8.397 | 65.494 | 17.673 | 1.00 | 52.91 | C |
| ATOM | 7186 | CD2 | TYR | B | 71 | 8.299 | 64.135 | 17.437 | 1.00 | 55.62 | C |
| ATOM | 7188 | C | TYR | B | 71 | 9.220 | 59.920 | 15.888 | 1.00 | 57.24 | C |
| ATOM | 7189 | O | TYR | B | 71 | 8.666 | 59.564 | 14.848 | 1.00 | 61.01 | O |
| ATOM | 7190 | N | THR | B | 72 | 9.633 | 59.045 | 16.816 | 1.00 | 56.65 | N |
| ATOM | 7192 | CA | THR | B | 72 | 9.338 | 57.601 | 16.757 | 1.00 | 49.69 | C |
| ATOM | 7194 | CB | THR | B | 72 | 10.143 | 56.812 | 17.843 | 1.00 | 45.99 | C |
| ATOM | 7196 | OG1 | THR | B | 72 | 11.515 | 56.708 | 17.485 | 1.00 | 41.99 | O |
| ATOM | 7198 | CG2 | THR | B | 72 | 9.736 | 55.355 | 17.907 | 1.00 | 48.51 | C |
| ATOM | 7202 | C | THR | B | 72 | 7.843 | 57.406 | 17.025 | 1.00 | 51.02 | C |
| ATOM | 7203 | O | THR | B | 72 | 7.208 | 56.549 | 16.431 | 1.00 | 52.84 | O |
| ATOM | 7204 | N | GLN | B | 73 | 7.303 | 58.198 | 17.946 | 1.00 | 48.27 | N |
| ATOM | 7206 | CA | GLN | B | 73 | 5.875 | 58.203 | 18.263 | 1.00 | 48.31 | C |
| ATOM | 7208 | CB | GLN | B | 73 | 5.581 | 57.409 | 19.545 | 1.00 | 51.35 | C |
| ATOM | 7211 | CG | GLN | B | 73 | 5.749 | 55.888 | 19.423 | 1.00 | 56.02 | C |
| ATOM | 7214 | CD | GLN | B | 73 | 4.857 | 55.255 | 18.344 | 1.00 | 61.90 | C |
| ATOM | 7215 | OE1 | GLN | B | 73 | 3.792 | 55.792 | 16.018 | 1.00 | 68.29 | O |
| ATOM | 7216 | NE2 | GLN | B | 73 | 5.295 | 54.124 | 17.783 | 1.00 | 62.95 | N |
| ATOM | 7219 | C | GLN | B | 73 | 5.437 | 59.654 | 18.470 | 1.00 | 49.42 | C |
| ATOM | 7220 | O | GLN | B | 73 | 5.747 | 60.278 | 19.508 | 1.00 | 46.54 | O |
| ATOM | 7221 | N | GLY | B | 74 | 4.722 | 60.214 | 17.499 | 1.00 | 48.91 | N |
| ATOM | 7223 | CA | GLY | B | 74 | 4.305 | 61.606 | 17.608 | 1.00 | 49.47 | C |
| ATOM | 7226 | C | GLY | B | 74 | 4.933 | 62.541 | 16.584 | 1.00 | 50.49 | C |
| ATOM | 7227 | O | GLY | B | 74 | 5.998 | 62.255 | 16.005 | 1.00 | 50.02 | O |
| ATOM | 7228 | N | LYS | B | 75 | 4.266 | 63.674 | 16.375 | 1.00 | 50.53 | N |
| ATOM | 7230 | CA | LYS | B | 75 | 4.660 | 64.627 | 15.342 | 1.00 | 52.28 | C |
| ATOM | 7232 | CB | LYS | B | 75 | 4.490 | 63.974 | 13.978 | 1.00 | 54.30 | C |
| ATOM | 7235 | CG | LYS | B | 75 | 3.047 | 63.970 | 13.545 | 1.00 | 57.04 | C |
| ATOM | 7238 | CD | LYS | B | 75 | 2.877 | 63.711 | 12.065 | 1.00 | 62.75 | C |
| ATOM | 7241 | CE | LYS | B | 75 | 3.286 | 62.309 | 11.659 | 1.00 | 69.74 | C |
| ATOM | 7244 | NZ | LYS | B | 75 | 3.245 | 62.144 | 10.151 | 1.00 | 75.75 | N |
| ATOM | 7248 | C | LYS | B | 75 | 3.767 | 65.863 | 15.374 | 1.00 | 48.92 | C |
| ATOM | 7249 | O | LYS | B | 75 | 2.624 | 65.773 | 15.785 | 1.00 | 50.04 | O |
| ATOM | 7250 | N | TRP | B | 76 | 4.288 | 67.005 | 14.939 | 1.00 | 47.21 | N |
| ATOM | 7252 | CA | TRP | B | 76 | 3.529 | 68.264 | 14.876 | 1.00 | 45.93 | C |
| ATOM | 7254 | CB | TRP | B | 76 | 3.623 | 69.023 | 16.212 | 1.00 | 44.91 | C |
| ATOM | 7257 | CG | TRP | B | 76 | 5.006 | 69.104 | 16.657 | 1.00 | 44.21 | C |
| ATOM | 7258 | CD1 | TRP | B | 76 | 5.635 | 68.312 | 17.578 | 1.00 | 48.52 | C |
| ATOM | 7260 | NE1 | TRP | B | 76 | 6.950 | 68.697 | 17.720 | 1.00 | 44.12 | N |
| ATOM | 7262 | CE2 | TRP | B | 76 | 7.190 | 69.744 | 16.878 | 1.00 | 40.53 | C |
| ATOM | 7263 | CD2 | TRP | B | 76 | 5.985 | 70.027 | 16.194 | 1.00 | 46.96 | C |
| ATOM | 7264 | CE3 | TRP | B | 76 | 5.967 | 71.072 | 15.266 | 1.00 | 44.10 | C |
| ATOM | 7266 | CZ3 | TRP | B | 76 | 7.090 | 71.766 | 15.055 | 1.00 | 33.58 | C |
| ATOM | 7268 | CH2 | TRP | B | 76 | 8.269 | 71.452 | 15.754 | 1.00 | 44.67 | C |
| ATOM | 7270 | CZ2 | TRP | B | 76 | 8.324 | 70.442 | 16.665 | 1.00 | 38.84 | C |
| ATOM | 7272 | C | TRP | B | 76 | 4.014 | 69.191 | 13.734 | 1.00 | 48.89 | C |
| ATOM | 7273 | O | TRP | B | 76 | 5.038 | 68.975 | 13.069 | 1.00 | 42.88 | O |
| ATOM | 7274 | N | GLU | B | 77 | 3.263 | 70.251 | 13.522 | 1.00 | 52.51 | N |
| ATOM | 7276 | CA | GLU | B | 77 | 3.588 | 71.211 | 12.483 | 1.00 | 54.19 | C |
| ATOM | 7278 | CB | GLU | B | 77 | 2.687 | 71.014 | 11.221 | 1.00 | 56.34 | C |
| ATOM | 7281 | CG | GLU | B | 77 | 2.928 | 71.973 | 10.024 | 1.00 | 65.54 | C |
| ATOM | 7284 | CD | GLU | B | 77 | 3.069 | 71.317 | 8.600 | 1.00 | 75.14 | C |
| ATOM | 7285 | OE1 | GLU | B | 77 | 2.692 | 70.105 | 8.385 | 1.00 | 75.56 | O |
| ATOM | 7286 | OE2 | GLU | B | 77 | 3.577 | 72.039 | 7.665 | 1.00 | 68.89 | O |
| ATOM | 7287 | C | GLU | B | 77 | 3.390 | 72.532 | 13.201 | 1.00 | 48.29 | C |
| ATOM | 7288 | O | GLU | B | 77 | 2.470 | 72.671 | 14.038 | 1.00 | 50.80 | O |
| ATOM | 7289 | N | GLY | B | 78 | 4.249 | 73.495 | 12.906 | 1.00 | 39.02 | N |
| ATOM | 7291 | CA | GLY | B | 78 | 4.116 | 74.794 | 13.527 | 1.00 | 38.17 | C |
| ATOM | 7294 | C | GLY | B | 78 | 4.560 | 75.872 | 12.574 | 1.00 | 41.86 | C |
| ATOM | 7295 | O | GLY | B | 78 | 4.622 | 75.629 | 11.360 | 1.00 | 42.61 | O |
| ATOM | 7296 | N | GLU | B | 79 | 4.865 | 77.040 | 13.150 | 1.00 | 40.98 | N |
| ATOM | 7298 | CA | GLU | B | 79 | 5.353 | 78.217 | 12.458 | 1.00 | 40.07 | C |
| ATOM | 7300 | CB | GLU | B | 79 | 4.182 | 79.179 | 12.341 | 1.00 | 45.01 | C |
| ATOM | 7303 | CG | GLU | B | 79 | 3.271 | 78.881 | 11.161 | 1.00 | 48.35 | C |
| ATOM | 7306 | CD | GLU | B | 79 | 1.879 | 79.445 | 11.376 | 1.00 | 59.96 | C |
| ATOM | 7307 | OE1 | GLU | B | 79 | 1.026 | 79.356 | 10.435 | 1.00 | 58.74 | O |
| ATOM | 7308 | OE2 | GLU | B | 79 | 1.656 | 79.975 | 12.506 | 1.00 | 62.96 | O |
| ATOM | 7309 | C | GLU | B | 79 | 6.534 | 78.904 | 13.206 | 1.00 | 39.29 | C |
| ATOM | 7310 | O | GLU | B | 79 | 6.387 | 79.304 | 14.373 | 1.00 | 42.71 | O |
| ATOM | 7311 | N | LEU | B | 80 | 7.685 | 79.057 | 12.520 | 1.00 | 40.28 | N |
| ATOM | 7313 | CA | LEU | B | 80 | 8.955 | 79.568 | 13.102 | 1.00 | 36.41 | C |
| ATOM | 7315 | CB | LEU | B | 80 | 10.154 | 79.219 | 12.177 | 1.00 | 36.64 | C |
| ATOM | 7318 | CG | LEU | B | 80 | 10.738 | 77.781 | 12.076 | 1.00 | 37.91 | C |
| ATOM | 7320 | CD1 | LEU | B | 80 | 11.526 | 77.548 | 10.745 | 1.00 | 39.77 | C |
| ATOM | 7324 | CD2 | LEU | B | 80 | 11.649 | 77.344 | 13.193 | 1.00 | 36.07 | C |
| ATOM | 7328 | C | LEU | B | 80 | 8.966 | 81.088 | 13.467 | 1.00 | 33.54 | C |
| ATOM | 7329 | O | LEU | B | 80 | 8.254 | 81.878 | 12.905 | 1.00 | 32.98 | O |
| ATOM | 7330 | N | GLY | B | 81 | 9.775 | 81.482 | 14.431 | 1.00 | 32.51 | N |
| ATOM | 7332 | CA | GLY | B | 81 | 9.853 | 82.869 | 14.860 | 1.00 | 36.03 | C |
| ATOM | 7335 | C | GLY | B | 81 | 11.029 | 82.934 | 15.848 | 1.00 | 42.04 | C |
| ATOM | 7336 | O | GLY | B | 81 | 11.757 | 81.938 | 15.970 | 1.00 | 29.06 | O |
| ATOM | 7337 | N | THR | B | 82 | 11.223 | 84.089 | 16.504 | 1.00 | 41.12 | N |
| ATOM | 7339 | CA | THR | B | 82 | 12.122 | 84.225 | 17.659 | 1.00 | 44.53 | C |
| ATOM | 7341 | CB | THR | B | 82 | 13.486 | 84.776 | 17.289 | 1.00 | 41.56 | C |
| ATOM | 7343 | OG1 | THR | B | 82 | 13.438 | 86.212 | 17.236 | 1.00 | 37.05 | O |
| ATOM | 7345 | CG2 | THR | B | 82 | 13.898 | 84.298 | 15.882 | 1.00 | 44.94 | C |
| ATOM | 7349 | C | THR | B | 82 | 11.512 | 65.157 | 18.726 | 1.00 | 47.70 | C |
| ATOM | 7350 | O | THR | B | 82 | 10.689 | 86.007 | 18.424 | 1.00 | 48.47 | O |
| ATOM | 7351 | N | ASP | B | 83 | 11.949 | 84.980 | 19.971 | 1.00 | 49.65 | N |
| ATOM | 7353 | CA | ASP | B | 83 | 11.536 | 85.805 | 21.078 | 1.00 | 43.80 | C |
| ATOM | 7355 | CB | ASP | B | 83 | 10.137 | 85.412 | 21.497 | 1.00 | 45.31 | C |
| ATOM | 7358 | CG | ASP | B | 83 | 9.416 | 86.517 | 22.208 | 1.00 | 48.10 | C |
| ATOM | 7359 | OD1 | ASP | B | 83 | 10.063 | 87.357 | 22.873 | 1.00 | 43.35 | O |
| ATOM | 7360 | OD2 | ASP | B | 83 | 8.174 | 86.617 | 22.144 | 1.00 | 55.68 | O |
| ATOM | 7361 | C | ASP | B | 83 | 12.515 | 85.545 | 22.220 | 1.00 | 44.30 | C |
| ATOM | 7362 | O | ASP | B | 83 | 13.317 | 84.618 | 22.164 | 1.00 | 42.87 | O |
| ATOM | 7363 | N | LEU | B | 84 | 12.431 | 86.359 | 23.264 | 1.00 | 44.69 | N |
| ATOM | 7365 | CA | LEU | B | 84 | 13.305 | 86.237 | 24.412 | 1.00 | 44.31 | C |
| ATOM | 7367 | CB | LEU | B | 84 | 13.298 | 87.556 | 25.184 | 1.00 | 42.41 | C |
| ATOM | 7370 | CG | LEU | B | 84 | 14.000 | 88.765 | 24.573 | 1.00 | 46.49 | C |
| ATOM | 7372 | CD1 | LEU | B | 84 | 14.187 | 89.925 | 25.584 | 1.00 | 45.21 | C |
| ATOM | 7376 | CD2 | LEU | B | 84 | 15.355 | 88.390 | 23.988 | 1.00 | 47.47 | C |
| ATOM | 7380 | C | LEU | B | 84 | 12.880 | 85.069 | 25.317 | 1.00 | 43.92 | C |
| ATOM | 7381 | O | LEU | B | 84 | 11.702 | 84.796 | 25.459 | 1.00 | 43.23 | O |
| ATOM | 7382 | N | VAL | B | 85 | 13.859 | 84.407 | 25.939 | 1.00 | 48.76 | N |
| ATOM | 7384 | CA | VAL | B | 85 | 13.639 | 83.210 | 26.747 | 1.00 | 48.52 | C |
| ATOM | 7386 | CB | VAL | B | 85 | 13.957 | 81.977 | 25.854 | 1.00 | 49.65 | C |
| ATOM | 7388 | CG1 | VAL | B | 85 | 13.559 | 80.668 | 26.519 | 1.00 | 54.85 | C |
| ATOM | 7392 | CG2 | VAL | B | 85 | 13.231 | 82.099 | 24.546 | 1.00 | 45.28 | C |
| ATOM | 7396 | C | VAL | B | 85 | 14.484 | 83.123 | 28.044 | 1.00 | 48.30 | C |
| ATOM | 7397 | O | VAL | B | 85 | 15.712 | 83.170 | 27.981 | 1.00 | 45.59 | O |
| ATOM | 7398 | N | SER | B | 86 | 13.825 | 83.000 | 29.206 | 1.00 | 49.13 | N |
| ATOM | 7400 | CA | SER | B | 86 | 14.515 | 82.778 | 30.497 | 1.00 | 50.27 | C |
| ATOM | 7402 | CB | SER | B | 86 | 14.143 | 83.844 | 31.493 | 1.00 | 52.28 | C |
| ATOM | 7405 | OG | SER | B | 86 | 13.971 | 85.077 | 30.826 | 1.00 | 61.45 | O |
| ATOM | 7407 | C | SER | B | 86 | 14.229 | 81.445 | 31.163 | 1.00 | 49.71 | C |
| ATOM | 7408 | O | SER | B | 86 | 13.262 | 80.765 | 30.859 | 1.00 | 54.75 | O |
| ATOM | 7409 | N | ILE | B | 87 | 15.095 | 81.078 | 32.089 | 1.00 | 52.73 | N |
| ATOM | 7411 | CA | ILE | B | 87 | 14.987 | 79.820 | 32.822 | 1.00 | 52.37 | C |
| ATOM | 7413 | CB | ILE | B | 87 | 16.176 | 78.977 | 32.494 | 1.00 | 50.27 | C |
| ATOM | 7415 | CG1 | ILE | B | 87 | 16.194 | 78.704 | 30.987 | 1.00 | 55.91 | C |
| ATOM | 7418 | CD1 | ILE | B | 87 | 17.502 | 78.078 | 30.465 | 1.00 | 57.49 | C |
| ATOM | 7422 | CG2 | ILE | B | 87 | 16.117 | 77.687 | 33.268 | 1.00 | 52.97 | C |
| ATOM | 7426 | C | ILE | B | 87 | 14.957 | 80.130 | 34.316 | 1.00 | 53.74 | C |
| ATOM | 7427 | O | ILE | B | 87 | 15.994 | 80.160 | 34.966 | 1.00 | 56.92 | O |
| ATOM | 7428 | N | PRO | B | 88 | 13.764 | 80.351 | 34.856 | 1.00 | 54.08 | N |
| ATOM | 7429 | CA | PRO | B | 88 | 13.610 | 80.874 | 36.215 | 1.00 | 55.87 | C |
| ATOM | 7431 | CB | PRO | B | 88 | 12.110 | 80.664 | 36.510 | 1.00 | 54.68 | C |
| ATOM | 7434 | CG | PRO | B | 88 | 11.476 | 80.768 | 35.191 | 1.00 | 51.31 | C |
| ATOM | 7437 | CD | PRO | B | 88 | 12.456 | 80.123 | 34.227 | 1.00 | 53.64 | C |
| ATOM | 7440 | C | PRO | B | 88 | 14.441 | 80.173 | 37.222 | 1.00 | 53.06 | C |
| ATOM | 7441 | O | PRO | B | 88 | 14.892 | 80.795 | 38.153 | 1.00 | 61.22 | O |
| ATOM | 7442 | N | HIS | B | 89 | 14.636 | 78.890 | 37.056 | 1.00 | 55.41 | N |
| ATOM | 7444 | CA | HIS | B | 89 | 15.464 | 78.155 | 38.000 | 1.00 | 57.00 | C |
| ATOM | 7446 | CB | HIS | B | 89 | 14.649 | 77.063 | 38.654 | 1.00 | 57.60 | C |
| ATOM | 7449 | CG | HIS | B | 89 | 13.583 | 77.600 | 39.539 | 1.00 | 62.81 | C |
| ATOM | 7450 | ND1 | HIS | B | 89 | 13.871 | 78.340 | 40.670 | 1.00 | 65.83 | N |
| ATOM | 7452 | CE1 | HIS | B | 89 | 12.738 | 78.695 | 41.251 | 1.00 | 69.42 | C |
| ATOM | 7454 | NE2 | HIS | B | 89 | 11.729 | 78.218 | 40.536 | 1.00 | 66.12 | N |
| ATOM | 7456 | CD2 | HIS | B | 89 | 12.232 | 77.532 | 39.456 | 1.00 | 60.92 | C |
| ATOM | 7458 | C | HIS | B | 89 | 16.689 | 77.559 | 37.354 | 1.00 | 56.54 | C |
| ATOM | 7459 | O | HIS | B | 89 | 17.155 | 76.516 | 37.779 | 1.00 | 57.96 | O |
| ATOM | 7460 | N | GLY | B | 90 | 17.200 | 78.221 | 36.323 | 1.00 | 57.33 | N |
| ATOM | 7462 | CA | GLY | B | 90 | 18.440 | 77.811 | 35.697 | 1.00 | 58.75 | C |
| ATOM | 7465 | C | GLY | B | 90 | 19.429 | 78.892 | 36.074 | 1.00 | 60.53 | C |
| ATOM | 7466 | O | GLY | B | 90 | 19.312 | 79.504 | 37.142 | 1.00 | 61.84 | O |
| ATOM | 7467 | N | PRO | B | 91 | 20.414 | 79.142 | 35.224 | 1.00 | 60.10 | N |
| ATOM | 7468 | CA | PRO | B | 91 | 21.300 | 80.262 | 35.461 | 1.00 | 57.74 | C |
| ATOM | 7470 | CB | PRO | B | 91 | 22.367 | 80.061 | 34.393 | 1.00 | 57.95 | C |
| ATOM | 7473 | CG | PRO | B | 91 | 21.614 | 79.508 | 33.279 | 1.00 | 56.85 | C |
| ATOM | 7476 | CD | PRO | B | 91 | 20.778 | 78.440 | 33.977 | 1.00 | 59.95 | C |
| ATOM | 7479 | C | PRO | B | 91 | 20.377 | 81.434 | 35.169 | 1.00 | 63.13 | C |
| ATOM | 7480 | O | PRO | B | 91 | 19.180 | 81.167 | 34.938 | 1.00 | 60.03 | O |
| ATOM | 7481 | N | GLN | B | 92 | 20.873 | 82.666 | 35.162 | 1.00 | 63.92 | N |
| ATOM | 7483 | CA | GLN | B | 92 | 19.996 | 83.819 | 35.023 | 1.00 | 66.19 | C |
| ATOM | 7485 | CB | GLN | B | 92 | 19.937 | 84.573 | 36.346 | 1.00 | 71.25 | C |
| ATOM | 7488 | CG | GLN | B | 92 | 19.439 | 83.713 | 37.525 | 1.00 | 73.31 | C |
| ATOM | 7491 | CD | GLN | B | 92 | 17.921 | 83.718 | 37.685 | 1.00 | 76.42 | C |
| ATOM | 7492 | OE1 | GLN | B | 92 | 17.345 | 82.717 | 38.150 | 1.00 | 76.72 | O |
| ATOM | 7493 | NE2 | GLN | B | 92 | 17.269 | 84.834 | 37.311 | 1.00 | 72.82 | N |
| ATOM | 7496 | C | GLN | B | 92 | 20.453 | 84.734 | 33.903 | 1.00 | 69.00 | C |
| ATOM | 7497 | O | GLN | B | 92 | 21.150 | 85.732 | 34.104 | 1.00 | 67.78 | O |
| ATOM | 7498 | N | VAL | B | 93 | 20.038 | 84.373 | 32.707 | 1.00 | 70.27 | N |
| ATOM | 7500 | CA | VAL | B | 93 | 20.402 | 85.094 | 31.520 | 1.00 | 70.88 | C |
| ATOM | 7502 | CB | VAL | B | 93 | 21.394 | 84.265 | 30.700 | 1.00 | 71.94 | C |
| ATOM | 7504 | CG1 | VAL | B | 93 | 22.762 | 84.286 | 31.339 | 1.00 | 72.94 | C |
| ATOM | 7508 | CG2 | VAL | B | 93 | 20.874 | 82.801 | 30.523 | 1.00 | 72.84 | C |
| ATOM | 7512 | C | VAL | B | 93 | 19.151 | 85.148 | 30.710 | 1.00 | 69.30 | C |
| ATOM | 7513 | O | VAL | B | 93 | 18.311 | 84.268 | 30.889 | 1.00 | 70.51 | O |
| ATOM | 7514 | N | THR | B | 94 | 18.997 | 86.150 | 29.841 | 1.00 | 65.09 | N |
| ATOM | 7516 | CA | THR | B | 94 | 17.947 | 86.039 | 28.832 | 1.00 | 65.10 | C |
| ATOM | 7518 | CB | THR | B | 94 | 17.092 | 87.296 | 28.739 | 1.00 | 68.17 | C |
| ATOM | 7520 | OG1 | THR | B | 94 | 16.798 | 87.798 | 30.049 | 1.00 | 65.92 | O |
| ATOM | 7522 | CG2 | THR | B | 94 | 15.731 | 86.972 | 28.133 | 1.00 | 72.53 | C |
| ATOM | 7526 | C | THR | B | 94 | 18.602 | 85.783 | 27.479 | 1.00 | 64.42 | C |
| ATOM | 7527 | O | THR | B | 94 | 19.532 | 86.496 | 27.088 | 1.00 | 69.43 | O |
| ATOM | 7528 | N | VAL | B | 95 | 18.140 | 84.772 | 26.756 | 1.00 | 57.14 | N |
| ATOM | 7530 | CA | VAL | B | 95 | 18.729 | 84.515 | 25.456 | 1.00 | 53.12 | C |
| ATOM | 7532 | CB | VAL | B | 95 | 19.394 | 83.121 | 25.323 | 1.00 | 50.84 | C |
| ATOM | 7534 | CG1 | VAL | B | 95 | 19.700 | 82.566 | 26.659 | 1.00 | 54.68 | C |
| ATOM | 7538 | CG2 | VAL | B | 95 | 18.555 | 82.158 | 24.577 | 1.00 | 46.33 | C |
| ATOM | 7542 | C | VAL | B | 95 | 17.635 | 84.620 | 24.454 | 1.00 | 52.68 | C |
| ATOM | 7543 | O | VAL | B | 95 | 16.475 | 84.377 | 24.770 | 1.00 | 47.03 | O |
| ATOM | 7544 | N | ARG | B | 96 | 18.010 | 84.998 | 23.242 | 1.00 | 50.05 | N |
| ATOM | 7546 | CA | ARG | B | 96 | 17.048 | 85.078 | 22.187 | 1.00 | 47.77 | C |
| ATOM | 7548 | CB | ARG | B | 96 | 17.343 | 86.245 | 21.248 | 1.00 | 45.70 | C |
| ATOM | 7551 | CG | ARG | B | 96 | 16.445 | 86.196 | 20.020 | 1.00 | 41.87 | C |
| ATOM | 7554 | CD | ARG | B | 96 | 16.267 | 87.486 | 19.279 | 1.00 | 44.39 | C |
| ATOM | 7557 | NE | ARG | B | 96 | 15.656 | 88.527 | 20.105 | 1.00 | 45.56 | N |
| ATOM | 7559 | CZ | ARG | B | 96 | 14.448 | 89.021 | 19.914 | 1.00 | 41.74 | C |
| ATOM | 7560 | NH1 | ARG | B | 96 | 13.712 | 88.593 | 18.920 | 1.00 | 37.61 | N |
| ATOM | 7563 | NH2 | ARG | B | 96 | 13.977 | 89.956 | 20.726 | 1.00 | 48.72 | N |
| ATOM | 7566 | C | ARG | B | 96 | 17.247 | 83.796 | 21.457 | 1.00 | 47.04 | C |
| ATOM | 7567 | O | ARG | B | 96 | 18.390 | 83.416 | 21.245 | 1.00 | 51.41 | O |
| ATOM | 7568 | N | ALA | B | 97 | 16.177 | 83.108 | 21.071 | 1.00 | 44.16 | N |
| ATOM | 7570 | CA | ALA | B | 97 | 16.390 | 81.906 | 20.291 | 1.00 | 43.51 | C |
| ATOM | 7572 | CB | ALA | B | 97 | 16.628 | 80.760 | 21.208 | 1.00 | 41.66 | C |
| ATOM | 7576 | C | ALA | B | 97 | 15.262 | 81.580 | 19.331 | 1.00 | 42.64 | C |
| ATOM | 7577 | O | ALA | B | 97 | 14.192 | 82.200 | 19.417 | 1.00 | 45.18 | O |
| ATOM | 7578 | N | ASN | B | 98 | 15.538 | 80.622 | 18.420 | 1.00 | 37.65 | N |
| ATOM | 7580 | CA | ASN | B | 98 | 14.564 | 80.108 | 17.495 | 1.00 | 37.82 | C |
| ATOM | 7582 | CB | ASN | B | 98 | 15.180 | 79.190 | 16.478 | 1.00 | 38.29 | C |
| ATOM | 7585 | CG | ASN | B | 98 | 16.227 | 79.845 | 15.654 | 1.00 | 41.90 | C |
| ATOM | 7586 | OD1 | ASN | B | 98 | 15.963 | 80.774 | 14.861 | 1.00 | 51.82 | O |
| ATOM | 7587 | ND2 | ASN | B | 98 | 17.441 | 79.362 | 15.803 | 1.00 | 44.46 | N |
| ATOM | 7590 | C | ASN | B | 98 | 13.573 | 79.313 | 18.299 | 1.00 | 39.41 | C |
| ATOM | 7591 | O | ASN | B | 98 | 13.936 | 78.699 | 19.292 | 1.00 | 41.27 | O |
| ATOM | 7592 | N | ILE | B | 99 | 12.325 | 79.318 | 17.851 | 1.00 | 44.08 | N |
| ATOM | 7594 | CA | ILE | B | 99 | 11.187 | 78.736 | 18.576 | 1.00 | 43.13 | C |
| ATOM | 7596 | CB | ILE | B | 99 | 10.469 | 79.758 | 19.384 | 1.00 | 40.30 | C |
| ATOM | 7598 | CG1 | ILE | B | 99 | 11.428 | 80.624 | 20.138 | 1.00 | 39.86 | C |
| ATOM | 7601 | CD1 | ILE | B | 99 | 10.726 | 81.360 | 21.242 | 1.00 | 36.21 | C |
| ATOM | 7605 | CG2 | ILE | B | 99 | 9.611 | 79.058 | 20.375 | 1.00 | 45.93 | C |
| ATOM | 7609 | C | ILE | B | 99 | 10.139 | 78.281 | 17.626 | 1.00 | 43.82 | C |
| ATOM | 7610 | O | ILE | B | 99 | 9.810 | 78.974 | 16.701 | 1.00 | 48.75 | O |
| ATOM | 7611 | N | ALA | B | 100 | 9.566 | 77.132 | 17.846 | 1.00 | 42.61 | N |
| ATOM | 7613 | CA | ALA | B | 100 | 8.610 | 76.737 | 16.883 | 1.00 | 43.98 | C |
| ATOM | 7615 | CB | ALA | B | 100 | 8.915 | 75.337 | 16.354 | 1.00 | 44.52 | C |
| ATOM | 7619 | C | ALA | B | 100 | 7.296 | 76.786 | 17.546 | 1.00 | 46.44 | C |
| ATOM | 7620 | O | ALA | B | 100 | 7.033 | 76.009 | 18.472 | 1.00 | 47.34 | O |
| ATOM | 7621 | N | ALA | B | 101 | 6.466 | 77.717 | 17.082 | 1.00 | 50.00 | N |
| ATOM | 7623 | CA | ALA | B | 101 | 5.076 | 77.790 | 17.525 | 1.00 | 44.40 | C |
| ATOM | 7625 | CB | ALA | B | 101 | 4.525 | 79.039 | 17.114 | 1.00 | 43.26 | C |
| ATOM | 7629 | C | ALA | B | 101 | 4.297 | 76.616 | 16.899 | 1.00 | 47.07 | C |
| ATOM | 7630 | O | ALA | B | 101 | 4.063 | 76.581 | 15.683 | 1.00 | 42.89 | O |
| ATOM | 7631 | N | ILE | B | 102 | 3.921 | 75.644 | 17.735 | 1.00 | 49.02 | N |
| ATOM | 7633 | CA | ILE | B | 102 | 3.167 | 74.464 | 17.301 | 1.00 | 46.91 | C |
| ATOM | 7635 | CB | ILE | B | 102 | 3.290 | 73.356 | 18.372 | 1.00 | 45.95 | C |
| ATOM | 7637 | CG1 | ILE | B | 102 | 4.715 | 72.820 | 18.475 | 1.00 | 47.92 | C |
| ATOM | 7640 | CD1 | ILE | B | 102 | 4.885 | 71.372 | 19.159 | 1.00 | 46.19 | C |
| ATOM | 7644 | CG2 | ILE | B | 102 | 2.340 | 72.210 | 18.062 | 1.00 | 47.95 | C |
| ATOM | 7648 | C | ILE | B | 102 | 1.655 | 74.792 | 17.051 | 1.00 | 44.85 | C |
| ATOM | 7649 | O | ILE | B | 102 | 0.976 | 75.227 | 17.958 | 1.00 | 42.16 | O |
| ATOM | 7650 | N | THR | B | 103 | 1.139 | 74.584 | 15.836 | 1.00 | 43.80 | N |
| ATOM | 7652 | CA | THR | B | 103 | −0.294 | 74.826 | 15.563 | 1.00 | 44.87 | C |
| ATOM | 7654 | CB | THR | B | 103 | −0.531 | 75.962 | 14.515 | 1.00 | 46.18 | C |
| ATOM | 7656 | OG1 | THR | B | 103 | −0.215 | 75.503 | 13.195 | 1.00 | 45.05 | O |
| ATOM | 7658 | CG2 | THR | B | 103 | 0.392 | 77.169 | 14.729 | 1.00 | 43.61 | C |
| ATOM | 7662 | C | THR | B | 103 | −1.088 | 73.582 | 15.122 | 1.00 | 45.54 | C |
| ATOM | 7663 | O | THR | B | 103 | −2.287 | 73.675 | 14.931 | 1.00 | 46.12 | O |
| ATOM | 7664 | N | GLU | B | 104 | −0.422 | 72.437 | 14.960 | 1.00 | 49.61 | N |
| ATOM | 7666 | CA | GLU | B | 104 | −1.069 | 71.158 | 14.595 | 1.00 | 51.72 | C |
| ATOM | 7668 | CB | GLU | B | 104 | −1.337 | 71.025 | 13.092 | 1.00 | 51.86 | C |
| ATOM | 7671 | CG | GLU | B | 104 | −2.389 | 71.919 | 12.460 | 1.00 | 58.09 | C |
| ATOM | 7674 | CD | GLU | B | 104 | −2.627 | 71.491 | 11.014 | 1.00 | 67.47 | C |
| ATOM | 7675 | OE1 | GLU | B | 104 | −2.393 | 70.288 | 10.764 | 1.00 | 78.27 | O |
| ATOM | 7676 | OE2 | GLU | B | 104 | −3.018 | 72.299 | 10.124 | 1.00 | 66.28 | O |
| ATOM | 7677 | C | GLU | B | 104 | −0.142 | 70.005 | 15.006 | 1.00 | 53.53 | C |
| ATOM | 7678 | O | GLU | B | 104 | 1.094 | 70.106 | 14.908 | 1.00 | 52.56 | O |
| ATOM | 7679 | N | SER | B | 105 | −0.726 | 68.903 | 15.452 | 1.00 | 54.92 | N |
| ATOM | 7681 | CA | SER | B | 105 | 0.068 | 67.817 | 16.038 | 1.00 | 54.07 | C |
| ATOM | 7683 | CB | SER | B | 105 | 0.436 | 68.122 | 17.491 | 1.00 | 53.33 | C |
| ATOM | 7686 | OG | SER | B | 105 | −0.746 | 68.387 | 18.230 | 1.00 | 54.72 | O |
| ATOM | 7688 | C | SER | B | 105 | −0.697 | 66.547 | 16.057 | 1.00 | 53.09 | C |
| ATOM | 7689 | O | SER | B | 105 | −1.903 | 66.512 | 15.853 | 1.00 | 53.09 | O |
| ATOM | 7690 | N | ASP | B | 106 | 0.021 | 65.483 | 16.321 | 1.00 | 51.65 | N |
| ATOM | 7692 | CA | ASP | B | 106 | −0.589 | 64.196 | 16.327 | 1.00 | 53.94 | C |
| ATOM | 7694 | CB | ASP | B | 106 | −0.600 | 63.656 | 14.878 | 1.00 | 56.95 | C |
| ATOM | 7697 | CG | ASP | B | 106 | −1.127 | 62.219 | 14.767 | 1.00 | 61.31 | C |
| ATOM | 7698 | OD1 | ASP | B | 106 | −0.982 | 61.455 | 15.749 | 1.00 | 68.69 | O |
| ATOM | 7699 | OD2 | ASP | B | 106 | −1.685 | 61.764 | 13.742 | 1.00 | 50.04 | O |
| ATOM | 7700 | C | ASP | B | 106 | 0.215 | 63.336 | 17.289 | 1.00 | 52.73 | C |
| ATOM | 7701 | O | ASP | B | 106 | 1.407 | 63.125 | 17.096 | 1.00 | 53.01 | O |
| ATOM | 7702 | N | LYS | B | 107 | −0.437 | 62.864 | 18.341 | 1.00 | 54.52 | N |
| ATOM | 7704 | CA | LYS | B | 107 | 0.169 | 61.898 | 19.239 | 1.00 | 56.18 | C |
| ATOM | 7706 | CB | LYS | B | 107 | 0.376 | 60.568 | 18.484 | 1.00 | 56.99 | C |
| ATOM | 7709 | CG | LYS | B | 107 | −0.897 | 59.700 | 18.297 | 1.00 | 62.79 | C |
| ATOM | 7712 | CD | LYS | B | 107 | −0.759 | 58.662 | 17.101 | 1.00 | 67.69 | C |
| ATOM | 7715 | CE | LYS | B | 107 | −1.476 | 57.266 | 17.326 | 1.00 | 70.59 | C |
| ATOM | 7718 | NZ | LYS | B | 107 | −0.573 | 56.024 | 17.628 | 1.00 | 61.25 | N |
| ATOM | 7722 | C | LYS | B | 107 | 1.496 | 62.465 | 19.798 | 1.00 | 56.67 | C |
| ATOM | 7723 | O | LYS | B | 107 | 2.496 | 61.745 | 19.939 | 1.00 | 57.97 | O |
| ATOM | 7724 | N | PHE | B | 108 | 1.485 | 63.757 | 20.131 | 1.00 | 54.71 | N |
| ATOM | 7726 | CA | PHE | B | 108 | 2.675 | 64.447 | 20.607 | 1.00 | 50.93 | C |
| ATOM | 7728 | CB | PHE | B | 108 | 2.881 | 65.761 | 19.867 | 1.00 | 49.68 | C |
| ATOM | 7731 | CG | PHE | B | 108 | 4.076 | 66.495 | 20.318 | 1.00 | 44.97 | C |
| ATOM | 7732 | CD1 | PHE | B | 108 | 5.317 | 65.919 | 20.213 | 1.00 | 49.71 | C |
| ATOM | 7734 | CE1 | PHE | B | 108 | 6.406 | 66.559 | 20.618 | 1.00 | 42.72 | C |
| ATOM | 7736 | CZ | PHE | B | 108 | 6.278 | 67.791 | 21.148 | 1.00 | 46.15 | C |
| ATOM | 7738 | CE2 | PHE | B | 108 | 5.061 | 68.387 | 21.259 | 1.00 | 42.41 | C |
| ATOM | 7740 | CD2 | PHE | B | 108 | 3.973 | 67.741 | 20.846 | 1.00 | 46.38 | C |
| ATOM | 7742 | C | PHE | B | 108 | 2.524 | 64.744 | 22.058 | 1.00 | 51.85 | C |
| ATOM | 7743 | O | PHE | B | 108 | 3.193 | 64.143 | 22.905 | 1.00 | 56.62 | O |
| ATOM | 7744 | N | PHE | B | 109 | 1.630 | 65.673 | 22.347 | 1.00 | 48.78 | N |
| ATOM | 7746 | CA | PHE | B | 109 | 1.324 | 66.029 | 23.717 | 1.00 | 49.69 | C |
| ATOM | 7748 | CB | PHE | B | 109 | 0.313 | 67.149 | 23.711 | 1.00 | 44.88 | C |
| ATOM | 7751 | CG | PHE | B | 109 | 0.729 | 68.284 | 22.858 | 1.00 | 44.87 | C |
| ATOM | 7752 | CD1 | PHE | B | 109 | −0.031 | 68.687 | 21.773 | 1.00 | 47.55 | C |
| ATOM | 7754 | CE1 | PHE | B | 109 | 0.373 | 69.771 | 20.978 | 1.00 | 46.68 | C |
| ATOM | 7756 | CZ | PHE | B | 109 | 1.541 | 70.428 | 21.267 | 1.00 | 44.40 | C |
| ATOM | 7758 | CE2 | PHE | B | 109 | 2.319 | 70.019 | 22.352 | 1.00 | 46.41 | C |
| ATOM | 7760 | CD2 | PHE | B | 109 | 1.909 | 68.958 | 23.132 | 1.00 | 48.50 | C |
| ATOM | 7762 | C | PHE | B | 109 | 0.752 | 64.780 | 24.364 | 1.00 | 53.27 | C |
| ATOM | 7763 | O | PHE | B | 109 | 0.386 | 63.840 | 23.661 | 1.00 | 53.57 | O |
| ATOM | 7764 | N | ILE | B | 110 | 0.677 | 64.764 | 25.690 | 1.00 | 57.18 | N |
| ATOM | 7766 | CA | ILE | B | 110 | 0.196 | 63.588 | 26.417 | 1.00 | 58.89 | C |
| ATOM | 7768 | CB | ILE | B | 110 | 1.353 | 62.759 | 26.974 | 1.00 | 61.55 | C |
| ATOM | 7770 | CG1 | ILE | B | 110 | 2.074 | 62.075 | 25.832 | 1.00 | 60.21 | C |
| ATOM | 7773 | CD1 | ILE | B | 110 | 3.487 | 61.911 | 26.109 | 1.00 | 60.39 | C |
| ATOM | 7777 | CG2 | ILE | B | 110 | 0.839 | 61.703 | 27.968 | 1.00 | 62.33 | C |
| ATOM | 7781 | C | ILE | B | 110 | −0.672 | 64.038 | 27.547 | 1.00 | 59.34 | C |
| ATOM | 7782 | O | ILE | B | 110 | −0.390 | 65.028 | 28.221 | 1.00 | 64.24 | O |
| ATOM | 7783 | N | GLN | B | 111 | −1.728 | 63.286 | 27.758 | 1.00 | 63.84 | N |
| ATOM | 7785 | CA | GLN | B | 111 | −2.744 | 63.660 | 28.706 | 1.00 | 65.98 | C |
| ATOM | 7787 | CB | GLN | B | 111 | −3.941 | 62.742 | 28.513 | 1.00 | 66.79 | C |
| ATOM | 7790 | CG | GLN | B | 111 | −5.248 | 63.200 | 29.097 | 1.00 | 70.55 | C |
| ATOM | 7793 | CD | GLN | B | 111 | −6.356 | 62.229 | 28.697 | 1.00 | 72.04 | C |
| ATOM | 7794 | OE1 | GLN | B | 111 | −6.292 | 61.033 | 29.023 | 1.00 | 74.63 | O |
| ATOM | 7795 | NE2 | GLN | B | 111 | −7.354 | 62.724 | 27.979 | 1.00 | 73.54 | N |
| ATOM | 7798 | C | GLN | B | 111 | −2.185 | 63.584 | 30.112 | 1.00 | 67.32 | C |
| ATOM | 7799 | O | GLN | B | 111 | −1.659 | 62.542 | 30.546 | 1.00 | 65.83 | O |
| ATOM | 7800 | N | GLY | B | 112 | −2.289 | 64.725 | 30.796 | 1.00 | 66.19 | N |
| ATOM | 7802 | CA | GLY | B | 112 | −1.840 | 64.879 | 32.169 | 1.00 | 67.48 | C |
| ATOM | 7805 | C | GLY | B | 112 | −0.336 | 65.050 | 32.368 | 1.00 | 65.65 | C |
| ATOM | 7806 | O | GLY | B | 112 | 0.120 | 65.257 | 33.492 | 1.00 | 68.14 | O |
| ATOM | 7807 | N | SER | B | 113 | 0.442 | 64.979 | 31.292 | 1.00 | 61.80 | N |
| ATOM | 7809 | CA | SER | B | 113 | 1.894 | 65.125 | 31.398 | 1.00 | 55.68 | C |
| ATOM | 7811 | CB | SER | B | 113 | 2.554 | 64.991 | 30.038 | 1.00 | 57.97 | C |
| ATOM | 7814 | OG | SER | B | 113 | 2.336 | 66.161 | 29.268 | 1.00 | 60.71 | O |
| ATOM | 7816 | C | SER | B | 113 | 2.335 | 66.437 | 31.994 | 1.00 | 48.28 | C |
| ATOM | 7817 | O | SER | B | 113 | 3.442 | 66.539 | 32.408 | 1.00 | 50.44 | O |
| ATOM | 7818 | N | ASN | B | 114 | 1.470 | 67.436 | 32.018 | 1.00 | 49.56 | N |
| ATOM | 7820 | CA | ASN | B | 114 | 1.752 | 68.743 | 32.593 | 1.00 | 47.18 | C |
| ATOM | 7822 | CB | ASN | B | 114 | 2.214 | 68.678 | 34.050 | 1.00 | 48.80 | C |
| ATOM | 7825 | CG | ASN | B | 114 | 1.907 | 69.976 | 34.796 | 1.00 | 50.93 | C |
| ATOM | 7826 | OD1 | ASN | B | 114 | 0.966 | 70.685 | 34.445 | 1.00 | 58.75 | O |
| ATOM | 7827 | ND2 | ASN | B | 114 | 2.688 | 70.294 | 35.807 | 1.00 | 52.05 | N |
| ATOM | 7830 | C | ASN | B | 114 | 2.746 | 69.569 | 31.814 | 1.00 | 47.99 | C |
| ATOM | 7831 | O | ASN | B | 114 | 3.077 | 70.715 | 32.203 | 1.00 | 47.48 | O |
| ATOM | 7832 | N | TRP | B | 115 | 3.266 | 69.060 | 30.717 | 1.00 | 45.35 | N |
| ATOM | 7834 | CA | TRP | B | 115 | 4.122 | 70.007 | 30.031 | 1.00 | 46.51 | C |
| ATOM | 7836 | CB | TRP | B | 115 | 5.503 | 69.497 | 29.653 | 1.00 | 38.21 | C |
| ATOM | 7839 | CG | TRP | B | 115 | 5.485 | 68.207 | 29.182 | 1.00 | 37.44 | C |
| ATOM | 7840 | CD1 | TRP | B | 115 | 5.538 | 67.090 | 29.927 | 1.00 | 45.91 | C |
| ATOM | 7842 | NE1 | TRP | B | 115 | 5.494 | 65.971 | 29.131 | 1.00 | 39.59 | N |
| ATOM | 7844 | CE2 | TRP | B | 115 | 5.411 | 66.381 | 27.830 | 1.00 | 39.30 | C |
| ATOM | 7845 | CD2 | TRP | B | 115 | 5.400 | 67.782 | 27.830 | 1.00 | 38.29 | C |
| ATOM | 7846 | CE3 | TRP | B | 115 | 5.325 | 68.447 | 26.611 | 1.00 | 36.20 | C |
| ATOM | 7848 | CZ3 | TRP | B | 115 | 5.254 | 67.738 | 25.483 | 1.00 | 32.01 | C |
| ATOM | 7850 | CH2 | TRP | B | 115 | 5.264 | 66.346 | 25.501 | 1.00 | 42.78 | C |
| ATOM | 7852 | CZ2 | TRP | B | 115 | 5.339 | 65.648 | 26.675 | 1.00 | 44.67 | C |
| ATOM | 7854 | C | TRP | B | 115 | 3.374 | 70.652 | 28.866 | 1.00 | 54.62 | C |
| ATOM | 7855 | O | TRP | B | 115 | 2.215 | 70.325 | 28.614 | 1.00 | 56.05 | O |
| ATOM | 7856 | N | GLU | B | 116 | 4.026 | 71.577 | 28.175 | 1.00 | 54.99 | N |
| ATOM | 7858 | CA | GLU | B | 116 | 3.410 | 72.257 | 27.038 | 1.00 | 52.06 | C |
| ATOM | 7860 | CB | GLU | B | 116 | 2.804 | 73.591 | 27.465 | 1.00 | 49.66 | C |
| ATOM | 7863 | CG | GLU | B | 116 | 1.880 | 73.473 | 28.675 | 1.00 | 57.34 | C |
| ATOM | 7866 | CD | GLU | B | 116 | 2.407 | 74.182 | 29.918 | 1.00 | 59.29 | C |
| ATOM | 7867 | OE1 | GLU | B | 116 | 1.937 | 73.833 | 31.026 | 1.00 | 65.68 | O |
| ATOM | 7868 | OE2 | GLU | B | 116 | 3.274 | 75.085 | 29.812 | 1.00 | 58.66 | O |
| ATOM | 7869 | C | GLU | B | 116 | 4.427 | 72.514 | 25.938 | 1.00 | 45.98 | C |
| ATOM | 7870 | O | GLU | B | 116 | 4.116 | 73.152 | 24.968 | 1.00 | 42.04 | O |
| ATOM | 7871 | N | GLY | B | 117 | 5.631 | 71.996 | 26.073 | 1.00 | 40.56 | N |
| ATOM | 7873 | CA | GLY | B | 117 | 6.659 | 72.302 | 25.096 | 1.00 | 45.15 | C |
| ATOM | 7876 | C | GLY | B | 117 | 7.792 | 71.319 | 25.176 | 1.00 | 44.40 | C |
| ATOM | 7877 | O | GLY | B | 117 | 7.805 | 70.461 | 26.074 | 1.00 | 47.93 | O |
| ATOM | 7878 | N | ILE | B | 118 | 8.725 | 71.426 | 24.236 | 1.00 | 40.44 | N |
| ATOM | 7880 | CA | ILE | B | 118 | 9.871 | 70.519 | 24.180 | 1.00 | 36.21 | C |
| ATOM | 7882 | CB | ILE | B | 118 | 9.674 | 69.514 | 23.086 | 1.00 | 35.00 | C |
| ATOM | 7884 | CG1 | ILE | B | 118 | 10.464 | 68.278 | 23.385 | 1.00 | 33.44 | C |
| ATOM | 7887 | CD1 | ILE | B | 118 | 10.403 | 67.207 | 22.303 | 1.00 | 34.03 | C |
| ATOM | 7891 | CG2 | ILE | B | 118 | 10.205 | 70.063 | 21.781 | 1.00 | 39.24 | C |
| ATOM | 7895 | C | ILE | B | 118 | 11.143 | 71.282 | 23.877 | 1.00 | 41.42 | C |
| ATOM | 7896 | O | ILE | B | 118 | 11.182 | 72.221 | 23.076 | 1.00 | 44.83 | O |
| ATOM | 7897 | N | LEU | B | 119 | 12.222 | 70.866 | 24.487 | 1.00 | 41.80 | N |
| ATOM | 7899 | CA | LEU | B | 119 | 13.436 | 71.619 | 24.365 | 1.00 | 39.40 | C |
| ATOM | 7901 | CB | LEU | B | 119 | 13.746 | 72.308 | 25.689 | 1.00 | 39.02 | C |
| ATOM | 7904 | CG | LEU | B | 119 | 15.223 | 72.554 | 25.963 | 1.00 | 48.91 | C |
| ATOM | 7906 | CD1 | LEU | B | 119 | 15.713 | 73.515 | 24.940 | 1.00 | 46.92 | C |
| ATOM | 7910 | CD2 | LEU | B | 119 | 15.530 | 73.056 | 27.398 | 1.00 | 50.56 | C |
| ATOM | 7914 | C | LEU | B | 119 | 14.367 | 70.537 | 24.053 | 1.00 | 38.35 | C |
| ATOM | 7915 | O | LEU | B | 119 | 14.783 | 69.813 | 24.919 | 1.00 | 43.76 | O |
| ATOM | 7916 | N | GLY | B | 120 | 14.670 | 70.384 | 22.787 | 1.00 | 40.04 | N |
| ATOM | 7918 | CA | GLY | B | 120 | 15.509 | 69.293 | 22.383 | 1.00 | 39.07 | C |
| ATOM | 7921 | C | GLY | B | 120 | 16.882 | 69.824 | 22.631 | 1.00 | 40.82 | C |
| ATOM | 7922 | O | GLY | B | 120 | 17.085 | 71.007 | 22.343 | 1.00 | 40.62 | O |
| ATOM | 7923 | N | LEU | B | 121 | 17.802 | 68.996 | 23.150 | 1.00 | 35.37 | N |
| ATOM | 7925 | CA | LEU | B | 121 | 19.142 | 69.463 | 23.439 | 1.00 | 33.34 | C |
| ATOM | 7927 | CB | LEU | B | 121 | 19.468 | 69.229 | 24.895 | 1.00 | 34.88 | C |
| ATOM | 7930 | CG | LEU | B | 121 | 18.605 | 69.997 | 25.920 | 1.00 | 41.96 | C |
| ATOM | 7932 | CD1 | LEU | B | 121 | 18.186 | 69.169 | 27.155 | 1.00 | 38.40 | C |
| ATOM | 7936 | CD2 | LEU | B | 121 | 19.280 | 71.249 | 26.382 | 1.00 | 41.64 | C |
| ATOM | 7940 | C | LEU | B | 121 | 20.169 | 68.759 | 22.554 | 1.00 | 38.26 | C |
| ATOM | 7941 | O | LEU | B | 121 | 21.348 | 68.835 | 22.820 | 1.00 | 37.48 | O |
| ATOM | 7942 | N | ALA | B | 122 | 19.740 | 68.086 | 21.489 | 1.00 | 40.79 | N |
| ATOM | 7944 | CA | ALA | B | 122 | 20.697 | 67.353 | 20.674 | 1.00 | 42.59 | C |
| ATOM | 7946 | CB | ALA | B | 122 | 20.045 | 66.267 | 19.949 | 1.00 | 49.22 | C |
| ATOM | 7950 | C | ALA | B | 122 | 21.322 | 68.323 | 19.728 | 1.00 | 45.18 | C |
| ATOM | 7951 | O | ALA | B | 122 | 21.205 | 69.515 | 19.938 | 1.00 | 45.85 | O |
| ATOM | 7952 | N | TYR | B | 123 | 22.006 | 67.848 | 18.697 | 1.00 | 47.39 | N |
| ATOM | 7954 | CA | TYR | B | 123 | 22.763 | 68.790 | 17.870 | 1.00 | 48.54 | C |
| ATOM | 7956 | CB | TYR | B | 123 | 24.118 | 68.208 | 17.447 | 1.00 | 50.84 | C |
| ATOM | 7959 | CG | TYR | B | 123 | 25.038 | 67.830 | 18.578 | 1.00 | 50.51 | C |
| ATOM | 7960 | CD1 | TYR | B | 123 | 24.905 | 66.616 | 19.214 | 1.00 | 52.07 | C |
| ATOM | 7962 | CE1 | TYR | B | 123 | 25.759 | 66.249 | 20.268 | 1.00 | 56.08 | C |
| ATOM | 7964 | CZ | TYR | B | 123 | 26.759 | 67.115 | 20.679 | 1.00 | 57.64 | C |
| ATOM | 7965 | OH | TYR | B | 123 | 27.589 | 66.754 | 21.710 | 1.00 | 62.04 | O |
| ATOM | 7967 | CE2 | TYR | B | 123 | 26.917 | 68.341 | 20.054 | 1.00 | 58.63 | C |
| ATOM | 7969 | CD2 | TYR | B | 123 | 26.051 | 68.692 | 19.001 | 1.00 | 56.37 | C |
| ATOM | 7971 | C | TYR | B | 123 | 21.979 | 69.223 | 16.661 | 1.00 | 43.80 | C |
| ATOM | 7972 | O | TYR | B | 123 | 20.957 | 68.637 | 16.350 | 1.00 | 50.79 | O |
| ATOM | 7973 | N | ALA | B | 124 | 22.477 | 70.250 | 15.982 | 1.00 | 43.43 | N |
| ATOM | 7975 | CA | ALA | B | 124 | 21.793 | 70.841 | 14.838 | 1.00 | 43.69 | C |
| ATOM | 7977 | CB | ALA | B | 124 | 22.566 | 72.036 | 14.319 | 1.00 | 46.07 | C |
| ATOM | 7981 | C | ALA | B | 124 | 21.612 | 69.869 | 13.717 | 1.00 | 42.56 | C |
| ATOM | 7982 | O | ALA | B | 124 | 20.594 | 69.882 | 13.067 | 1.00 | 42.47 | O |
| ATOM | 7983 | N | GLU | B | 125 | 22.582 | 69.006 | 13.476 | 1.00 | 44.82 | N |
| ATOM | 7985 | CA | GLU | B | 125 | 22.469 | 68.163 | 12.296 | 1.00 | 47.99 | C |
| ATOM | 7987 | CB | GLU | B | 125 | 23.515 | 67.059 | 12.235 | 1.00 | 49.37 | C |
| ATOM | 7990 | CG | GLU | B | 125 | 22.957 | 65.812 | 11.577 | 1.00 | 57.34 | C |
| ATOM | 7993 | CD | GLU | B | 125 | 23.551 | 65.493 | 10.224 | 1.00 | 64.77 | C |
| ATOM | 7994 | OE1 | GLU | B | 125 | 24.491 | 64.674 | 10.186 | 1.00 | 71.72 | O |
| ATOM | 7995 | OE2 | GLU | B | 125 | 23.068 | 66.040 | 9.201 | 1.00 | 74.39 | O |
| ATOM | 7996 | C | GLU | B | 125 | 21.107 | 67.553 | 12.132 | 1.00 | 44.92 | C |
| ATOM | 7997 | O | GLU | B | 125 | 20.708 | 67.270 | 11.008 | 1.00 | 47.91 | O |
| ATOM | 7998 | N | ILE | B | 126 | 20.381 | 67.329 | 13.219 | 1.00 | 43.01 | N |
| ATOM | 8000 | CA | ILE | B | 126 | 19.116 | 66.641 | 13.067 | 1.00 | 40.58 | C |
| ATOM | 8002 | CB | ILE | B | 126 | 19.078 | 65.412 | 13.969 | 1.00 | 39.84 | C |
| ATOM | 8004 | CG1 | ILE | B | 126 | 18.870 | 65.776 | 15.413 | 1.00 | 43.52 | C |
| ATOM | 8007 | CD1 | ILE | B | 126 | 18.662 | 64.549 | 16.288 | 1.00 | 44.92 | C |
| ATOM | 8011 | CG2 | ILE | B | 126 | 20.372 | 64.751 | 13.908 | 1.00 | 40.38 | C |
| ATOM | 8015 | C | ILE | B | 126 | 17.962 | 67.565 | 13.315 | 1.00 | 42.42 | C |
| ATOM | 8016 | O | ILE | B | 126 | 16.823 | 67.138 | 13.565 | 1.00 | 40.90 | O |
| ATOM | 8017 | N | ALA | B | 127 | 18.250 | 68.853 | 13.236 | 1.00 | 42.86 | N |
| ATOM | 8019 | CA | ALA | B | 127 | 17.202 | 69.836 | 13.404 | 1.00 | 42.99 | C |
| ATOM | 8021 | CB | ALA | B | 127 | 17.753 | 71.130 | 13.922 | 1.00 | 44.98 | C |
| ATOM | 8025 | C | ALA | B | 127 | 16.525 | 70.059 | 12.091 | 1.00 | 41.47 | C |
| ATOM | 8026 | O | ALA | B | 127 | 17.105 | 69.882 | 11.050 | 1.00 | 39.97 | O |
| ATOM | 8027 | N | ARG | B | 128 | 15.272 | 70.444 | 12.165 | 1.00 | 44.79 | N |
| ATOM | 8029 | CA | ARG | B | 128 | 14.517 | 70.755 | 10.990 | 1.00 | 43.13 | C |
| ATOM | 8031 | CB | ARG | B | 128 | 13.282 | 69.894 | 10.960 | 1.00 | 42.01 | C |
| ATOM | 8034 | CG | ARG | B | 128 | 13.574 | 68.400 | 11.101 | 1.00 | 49.12 | C |
| ATOM | 8037 | CD | ARG | B | 128 | 13.536 | 67.609 | 9.795 | 1.00 | 52.69 | C |
| ATOM | 8040 | NE | ARG | B | 128 | 13.330 | 66.174 | 10.019 | 1.00 | 58.75 | N |
| ATOM | 8042 | CZ | ARG | B | 128 | 12.511 | 65.403 | 9.306 | 1.00 | 61.67 | C |
| ATOM | 8043 | NH1 | ARG | B | 128 | 11.809 | 65.915 | 8.308 | 1.00 | 58.06 | N |
| ATOM | 8046 | NH2 | ARG | B | 128 | 12.396 | 64.110 | 9.592 | 1.00 | 62.03 | N |
| ATOM | 8049 | C | ARG | B | 128 | 14.154 | 72.208 | 11.134 | 1.00 | 41.86 | C |
| ATOM | 8050 | O | ARG | B | 128 | 14.131 | 72.729 | 12.258 | 1.00 | 39.07 | O |
| ATOM | 8051 | N | PRO | B | 129 | 13.883 | 72.863 | 10.013 | 1.00 | 42.22 | N |
| ATOM | 8052 | CA | PRO | B | 129 | 13.958 | 72.233 | 8.694 | 1.00 | 43.56 | C |
| ATOM | 8054 | CB | PRO | B | 129 | 13.390 | 73.274 | 7.755 | 1.00 | 45.93 | C |
| ATOM | 8057 | CG | PRO | B | 129 | 12.704 | 74.249 | 8.633 | 1.00 | 47.55 | C |
| ATOM | 8060 | CD | PRO | B | 129 | 13.465 | 74.266 | 9.924 | 1.00 | 43.06 | C |
| ATOM | 8063 | C | PRO | B | 129 | 15.348 | 71.893 | 8.290 | 1.00 | 45.94 | C |
| ATOM | 8064 | O | PRO | B | 129 | 15.524 | 71.066 | 7.396 | 1.00 | 49.70 | O |
| ATOM | 8065 | N | ASP | B | 130 | 16.340 | 72.488 | 8.929 | 1.00 | 51.65 | N |
| ATOM | 8067 | CA | ASP | B | 130 | 17.715 | 72.086 | 8.642 | 1.00 | 53.34 | C |
| ATOM | 8069 | CB | ASP | B | 130 | 18.119 | 72.654 | 7.330 | 1.00 | 55.91 | C |
| ATOM | 8072 | CG | ASP | B | 130 | 18.180 | 74.138 | 7.387 | 1.00 | 61.07 | C |
| ATOM | 8073 | OD1 | ASP | B | 130 | 17.510 | 74.791 | 6.545 | 1.00 | 72.80 | O |
| ATOM | 8074 | OD2 | ASP | B | 130 | 18.856 | 74.738 | 8.265 | 1.00 | 52.07 | O |
| ATOM | 8075 | C | ASP | B | 130 | 18.748 | 72.613 | 9.600 | 1.00 | 55.45 | C |
| ATOM | 8076 | O | ASP | B | 130 | 18.515 | 73.579 | 10.342 | 1.00 | 58.60 | O |
| ATOM | 8077 | N | ASP | B | 131 | 19.918 | 71.989 | 9.529 | 1.00 | 53.37 | N |
| ATOM | 8079 | CA | ASP | B | 131 | 21.056 | 72.356 | 10.347 | 1.00 | 52.86 | C |
| ATOM | 8081 | CB | ASP | B | 131 | 22.321 | 71.615 | 9.849 | 1.00 | 56.83 | C |
| ATOM | 8084 | CG | ASP | B | 131 | 22.898 | 72.170 | 8.499 | 1.00 | 67.88 | C |
| ATOM | 8085 | OD1 | ASP | B | 131 | 22.369 | 73.164 | 7.913 | 1.00 | 71.66 | O |
| ATOM | 8086 | OD2 | ASP | B | 131 | 23.911 | 71.651 | 7.950 | 1.00 | 67.67 | O |
| ATOM | 8087 | C | ASP | B | 131 | 21.294 | 73.855 | 10.486 | 1.00 | 47.33 | C |
| ATOM | 8088 | O | ASP | B | 131 | 22.103 | 74.261 | 11.276 | 1.00 | 55.44 | O |
| ATOM | 8089 | N | SER | B | 132 | 20.599 | 74.692 | 9.743 | 1.00 | 49.22 | N |
| ATOM | 8091 | CA | SER | B | 132 | 20.799 | 76.144 | 9.863 | 1.00 | 51.59 | C |
| ATOM | 8093 | CB | SER | B | 132 | 20.431 | 76.880 | 8.576 | 1.00 | 56.09 | C |
| ATOM | 8096 | OG | SER | B | 132 | 19.064 | 77.316 | 8.672 | 1.00 | 56.61 | O |
| ATOM | 8098 | C | SER | B | 132 | 19.910 | 76.773 | 10.877 | 1.00 | 48.16 | C |
| ATOM | 8099 | O | SER | B | 132 | 19.967 | 77.966 | 11.030 | 1.00 | 48.29 | O |
| ATOM | 8100 | N | LEU | B | 133 | 19.044 | 75.996 | 11.514 | 1.00 | 51.70 | N |
| ATOM | 8102 | CA | LEU | B | 133 | 18.210 | 76.511 | 12.594 | 1.00 | 48.34 | C |
| ATOM | 8104 | CB | LEU | B | 133 | 16.784 | 75.967 | 12.530 | 1.00 | 50.22 | C |
| ATOM | 8107 | CG | LEU | B | 133 | 15.867 | 76.770 | 13.479 | 1.00 | 50.22 | C |
| ATOM | 8109 | CD1 | LEU | B | 133 | 15.291 | 77.914 | 12.692 | 1.00 | 51.60 | C |
| ATOM | 8113 | CD2 | LEU | B | 133 | 14.749 | 75.979 | 14.133 | 1.00 | 46.25 | C |
| ATOM | 8117 | C | LEU | B | 133 | 18.841 | 76.165 | 13.947 | 1.00 | 48.97 | C |
| ATOM | 8118 | O | LEU | B | 133 | 18.549 | 75.164 | 14.561 | 1.00 | 55.14 | O |
| ATOM | 8119 | N | GLU | B | 134 | 19.728 | 77.021 | 14.398 | 1.00 | 51.87 | N |
| ATOM | 8121 | CA | GLU | B | 134 | 20.411 | 76.871 | 15.673 | 1.00 | 49.82 | C |
| ATOM | 8123 | CB | GLU | B | 134 | 21.132 | 78.206 | 15.930 | 1.00 | 52.58 | C |
| ATOM | 8126 | CG | GLU | B | 134 | 21.942 | 78.394 | 17.194 | 1.00 | 57.11 | C |
| ATOM | 8129 | CD | GLU | B | 134 | 22.920 | 79.568 | 17.051 | 1.00 | 62.70 | C |
| ATOM | 8130 | OE1 | GLU | B | 134 | 23.903 | 79.441 | 16.281 | 1.00 | 65.85 | O |
| ATOM | 8131 | OE2 | GLU | B | 134 | 22.717 | 80.622 | 17.696 | 1.00 | 63.04 | O |
| ATOM | 8132 | C | GLU | B | 134 | 19.462 | 76.499 | 16.822 | 1.00 | 46.15 | C |
| ATOM | 8133 | O | GLU | B | 134 | 18.509 | 77.230 | 17.070 | 1.00 | 40.78 | O |
| ATOM | 8134 | N | PRO | B | 135 | 19.735 | 75.368 | 17.494 | 1.00 | 39.43 | N |
| ATOM | 8135 | CA | PRO | B | 135 | 19.040 | 74.917 | 18.710 | 1.00 | 40.62 | C |
| ATOM | 8137 | CB | PRO | B | 135 | 19.597 | 73.515 | 18.944 | 1.00 | 39.60 | C |
| ATOM | 8140 | CG | PRO | B | 135 | 20.409 | 73.187 | 17.783 | 1.00 | 40.53 | C |
| ATOM | 8143 | CD | PRO | B | 135 | 20.750 | 74.409 | 17.082 | 1.00 | 41.45 | C |
| ATOM | 8146 | C | PRO | B | 135 | 19.301 | 75.722 | 19.982 | 1.00 | 39.91 | C |
| ATOM | 8147 | O | PRO | B | 135 | 20.327 | 76.366 | 20.169 | 1.00 | 42.53 | O |
| ATOM | 8148 | N | PHE | B | 136 | 18.361 | 75.638 | 20.906 | 1.00 | 44.02 | N |
| ATOM | 8150 | CA | PHE | B | 136 | 18.396 | 76.470 | 22.119 | 1.00 | 44.27 | C |
| ATOM | 8152 | CB | PHE | B | 136 | 17.279 | 76.100 | 23.088 | 1.00 | 41.68 | C |
| ATOM | 8155 | CG | PHE | B | 136 | 17.309 | 76.929 | 24.293 | 1.00 | 39.04 | C |
| ATOM | 8156 | CD1 | PHE | B | 136 | 16.639 | 78.098 | 24.334 | 1.00 | 40.01 | C |
| ATOM | 8158 | CE1 | PHE | B | 136 | 16.683 | 78.881 | 25.442 | 1.00 | 43.44 | C |
| ATOM | 8160 | CZ | PHE | B | 136 | 17.417 | 78.488 | 26.517 | 1.00 | 46.05 | C |
| ATOM | 8162 | CE2 | PHE | B | 136 | 18.113 | 77.300 | 26.477 | 1.00 | 43.71 | C |
| ATOM | 8164 | CD2 | PHE | B | 136 | 18.056 | 76.545 | 25.374 | 1.00 | 44.61 | C |
| ATOM | 8166 | C | PHE | B | 136 | 19.672 | 76.460 | 22.926 | 1.00 | 42.84 | C |
| ATOM | 8167 | O | PHE | B | 136 | 20.091 | 77.478 | 23.427 | 1.00 | 44.70 | O |
| ATOM | 8168 | N | PHE | B | 137 | 20.282 | 75.304 | 23.088 | 1.00 | 44.28 | N |
| ATOM | 8170 | CA | PHE | B | 137 | 21.496 | 75.261 | 23.888 | 1.00 | 45.73 | C |
| ATOM | 8172 | CB | PHE | B | 137 | 21.819 | 73.830 | 24.336 | 1.00 | 46.94 | C |
| ATOM | 8175 | CG | PHE | B | 137 | 22.697 | 73.789 | 25.521 | 1.00 | 45.95 | C |
| ATOM | 8176 | CD1 | PHE | B | 137 | 22.194 | 73.993 | 26.756 | 1.00 | 47.44 | C |
| ATOM | 8178 | CE1 | PHE | B | 137 | 23.015 | 73.972 | 27.830 | 1.00 | 47.99 | C |
| ATOM | 8180 | CZ | PHE | B | 137 | 24.338 | 73.756 | 27.680 | 1.00 | 36.10 | C |
| ATOM | 8182 | CE2 | PHE | B | 137 | 24.842 | 73.561 | 26.482 | 1.00 | 37.75 | C |
| ATOM | 8184 | CD2 | PHE | B | 137 | 24.041 | 73.575 | 25.394 | 1.00 | 45.95 | C |
| ATOM | 8186 | C | PHE | B | 137 | 22.696 | 75.938 | 23.216 | 1.00 | 43.32 | C |
| ATOM | 8187 | O | PHE | B | 137 | 23.468 | 76.599 | 23.887 | 1.00 | 42.97 | O |
| ATOM | 8188 | N | ASP | B | 138 | 22.843 | 75.783 | 21.895 | 1.00 | 44.54 | N |
| ATOM | 8190 | CA | ASP | B | 138 | 23.891 | 76.481 | 21.156 | 1.00 | 37.01 | C |
| ATOM | 8192 | CB | ASP | B | 138 | 23.736 | 76.253 | 19.706 | 1.00 | 35.76 | C |
| ATOM | 8195 | CG | ASP | B | 138 | 24.247 | 74.993 | 19.282 | 1.00 | 41.96 | C |
| ATOM | 8196 | OD1 | ASP | B | 138 | 25.187 | 75.024 | 18.467 | 1.00 | 61.10 | O |
| ATOM | 8197 | OD2 | ASP | B | 138 | 23.796 | 73.908 | 19.672 | 1.00 | 56.89 | O |
| ATOM | 8198 | C | ASP | B | 138 | 23.788 | 77.995 | 21.316 | 1.00 | 43.61 | C |
| ATOM | 8199 | O | ASP | B | 138 | 24.743 | 78.650 | 21.697 | 1.00 | 47.84 | O |
| ATOM | 8200 | N | SER | B | 139 | 22.626 | 78.554 | 20.991 | 1.00 | 46.65 | N |
| ATOM | 8202 | CA | SER | B | 139 | 22.433 | 79.979 | 21.115 | 1.00 | 48.26 | C |
| ATOM | 8204 | CB | SER | B | 139 | 21.004 | 80.383 | 20.742 | 1.00 | 51.54 | C |
| ATOM | 8207 | OG | SER | B | 139 | 20.647 | 79.872 | 19.469 | 1.00 | 51.53 | O |
| ATOM | 8209 | C | SER | B | 139 | 22.691 | 80.372 | 22.526 | 1.00 | 48.43 | C |
| ATOM | 8210 | O | SER | B | 139 | 23.245 | 81.423 | 22.774 | 1.00 | 49.51 | O |
| ATOM | 8211 | N | LEU | B | 140 | 22.286 | 79.537 | 23.470 | 1.00 | 52.62 | N |
| ATOM | 8213 | CA | LEU | B | 140 | 22.480 | 79.895 | 24.874 | 1.00 | 56.48 | C |
| ATOM | 8215 | CB | LEU | B | 140 | 21.930 | 78.831 | 25.813 | 1.00 | 56.51 | C |
| ATOM | 8218 | CG | LEU | B | 140 | 22.375 | 79.194 | 27.234 | 1.00 | 54.72 | C |
| ATOM | 8220 | CD1 | LEU | B | 140 | 21.590 | 80.369 | 27.719 | 1.00 | 55.46 | C |
| ATOM | 8224 | CD2 | LEU | B | 140 | 22.222 | 78.095 | 28.210 | 1.00 | 57.48 | C |
| ATOM | 8228 | C | LEU | B | 140 | 23.955 | 80.086 | 25.211 | 1.00 | 57.93 | C |
| ATOM | 8229 | O | LEU | B | 140 | 24.333 | 81.037 | 25.897 | 1.00 | 59.89 | O |
| ATOM | 8230 | N | VAL | B | 141 | 24.793 | 79.168 | 24.751 | 1.00 | 55.68 | N |
| ATOM | 8232 | CA | VAL | B | 141 | 26.196 | 79.300 | 25.021 | 1.00 | 54.58 | C |
| ATOM | 8234 | CB | VAL | B | 141 | 26.883 | 78.025 | 24.692 | 1.00 | 58.69 | C |
| ATOM | 8236 | CG1 | VAL | B | 141 | 28.370 | 78.251 | 24.440 | 1.00 | 58.03 | C |
| ATOM | 8240 | CG2 | VAL | B | 141 | 26.664 | 77.039 | 25.839 | 1.00 | 61.23 | C |
| ATOM | 8244 | C | VAL | B | 141 | 26.822 | 80.452 | 24.243 | 1.00 | 57.01 | C |
| ATOM | 8245 | O | VAL | B | 141 | 27.587 | 81.229 | 24.817 | 1.00 | 58.33 | O |
| ATOM | 8246 | N | LYS | B | 142 | 26.517 | 80.585 | 22.948 | 1.00 | 54.66 | N |
| ATOM | 8248 | CA | LYS | B | 142 | 27.109 | 81.677 | 22.188 | 1.00 | 51.01 | C |
| ATOM | 8250 | CB | LYS | B | 142 | 26.710 | 81.694 | 20.673 | 1.00 | 47.84 | C |
| ATOM | 8253 | CG | LYS | B | 142 | 27.455 | 80.681 | 19.723 | 1.00 | 48.68 | C |
| ATOM | 8256 | CD | LYS | B | 142 | 26.743 | 80.426 | 18.329 | 1.00 | 54.09 | C |
| ATOM | 8259 | CE | LYS | B | 142 | 27.275 | 79.184 | 17.438 | 1.00 | 59.89 | C |
| ATOM | 8262 | NZ | LYS | B | 142 | 26.633 | 77.779 | 17.577 | 1.00 | 63.41 | N |
| ATOM | 8266 | C | LYS | B | 142 | 26.755 | 82.966 | 22.952 | 1.00 | 49.96 | C |
| ATOM | 8267 | O | LYS | B | 142 | 27.616 | 83.791 | 23.156 | 1.00 | 61.21 | O |
| ATOM | 8268 | N | GLN | B | 143 | 25.521 | 83.104 | 23.424 | 1.00 | 50.78 | N |
| ATOM | 8270 | CA | GLN | B | 143 | 24.976 | 84.369 | 24.029 | 1.00 | 49.37 | C |
| ATOM | 8272 | CB | GLN | B | 143 | 23.414 | 84.421 | 23.829 | 1.00 | 45.79 | C |
| ATOM | 8275 | CG | GLN | B | 143 | 22.945 | 84.825 | 22.372 | 1.00 | 47.31 | C |
| ATOM | 8278 | CD | GLN | B | 143 | 21.414 | 84.810 | 22.107 | 1.00 | 47.17 | C |
| ATOM | 8279 | OE1 | GLN | B | 143 | 20.610 | 85.248 | 22.936 | 1.00 | 50.01 | O |
| ATOM | 8280 | NE2 | GLN | B | 143 | 21.028 | 84.304 | 20.937 | 1.00 | 42.57 | N |
| ATOM | 8283 | C | GLN | B | 143 | 25.276 | 84.719 | 25.510 | 1.00 | 49.46 | C |
| ATOM | 8284 | O | GLN | B | 143 | 24.919 | 85.789 | 25.961 | 1.00 | 53.59 | O |
| ATOM | 8285 | N | THR | B | 144 | 25.887 | 83.831 | 26.275 | 1.00 | 54.02 | N |
| ATOM | 8287 | CA | THR | B | 144 | 26.271 | 84.125 | 27.662 | 1.00 | 56.45 | C |
| ATOM | 8289 | CB | THR | B | 144 | 25.218 | 83.587 | 28.639 | 1.00 | 58.45 | C |
| ATOM | 8291 | OG1 | THR | B | 144 | 25.574 | 82.273 | 29.043 | 1.00 | 57.95 | O |
| ATOM | 8293 | CG2 | THR | B | 144 | 23.847 | 83.426 | 27.968 | 1.00 | 57.06 | C |
| ATOM | 8297 | C | THR | B | 144 | 27.659 | 83.476 | 27.925 | 1.00 | 62.87 | C |
| ATOM | 8298 | O | THR | B | 144 | 28.354 | 83.092 | 26.977 | 1.00 | 65.39 | O |
| ATOM | 8299 | N | HIS | B | 145 | 28.091 | 83.348 | 29.174 | 1.00 | 65.98 | N |
| ATOM | 8301 | CA | HIS | B | 145 | 29.387 | 82.706 | 29.445 | 1.00 | 74.26 | C |
| ATOM | 8303 | CB | HIS | B | 145 | 30.175 | 83.559 | 30.474 | 1.00 | 80.56 | C |
| ATOM | 8306 | CG | HIS | B | 145 | 30.447 | 84.988 | 30.036 | 1.00 | 86.80 | C |
| ATOM | 8307 | ND1 | HIS | B | 145 | 29.853 | 86.089 | 30.629 | 1.00 | 87.60 | N |
| ATOM | 8309 | CE1 | HIS | B | 145 | 30.285 | 87.194 | 30.045 | 1.00 | 87.90 | C |
| ATOM | 8311 | NE2 | HIS | B | 145 | 31.142 | 86.856 | 29.099 | 1.00 | 89.90 | N |
| ATOM | 8313 | CD2 | HIS | B | 145 | 31.263 | 85.486 | 29.073 | 1.00 | 88.35 | C |
| ATOM | 8315 | C | HIS | B | 145 | 29.243 | 81.228 | 29.905 | 1.00 | 71.71 | C |
| ATOM | 8316 | O | HIS | B | 145 | 30.212 | 80.600 | 30.363 | 1.00 | 71.89 | O |
| ATOM | 8317 | N | VAL | B | 146 | 28.031 | 80.682 | 29.754 | 1.00 | 66.58 | N |
| ATOM | 8319 | CA | VAL | B | 146 | 27.678 | 79.350 | 30.230 | 1.00 | 58.14 | C |
| ATOM | 8321 | CB | VAL | B | 146 | 26.233 | 79.064 | 29.905 | 1.00 | 59.42 | C |
| ATOM | 8323 | CG1 | VAL | B | 146 | 25.936 | 77.582 | 29.997 | 1.00 | 62.47 | C |
| ATOM | 6327 | CG2 | VAL | B | 146 | 25.357 | 79.864 | 30.838 | 1.00 | 57.89 | C |
| ATOM | 8331 | C | VAL | B | 146 | 28.537 | 78.328 | 29.562 | 1.00 | 54.48 | C |
| ATOM | 8332 | O | VAL | B | 146 | 28.531 | 78.205 | 28.337 | 1.00 | 59.51 | O |
| ATOM | 8333 | N | PRO | B | 147 | 29.288 | 77.580 | 30.359 | 1.00 | 51.96 | N |
| ATOM | 8334 | CA | PRO | B | 147 | 30.138 | 76.562 | 29.777 | 1.00 | 49.03 | C |
| ATOM | 8336 | CB | PRO | B | 147 | 30.757 | 75.883 | 30.985 | 1.00 | 46.35 | C |
| ATOM | 8339 | CG | PRO | B | 147 | 30.725 | 76.871 | 32.008 | 1.00 | 44.84 | C |
| ATOM | 8342 | CD | PRO | B | 147 | 29.404 | 77.576 | 31.827 | 1.00 | 51.63 | C |
| ATOM | 8345 | C | PRO | B | 147 | 29.250 | 75.614 | 29.019 | 1.00 | 51.04 | C |
| ATOM | 8346 | O | PRO | B | 147 | 28.120 | 75.312 | 29.390 | 1.00 | 56.78 | O |
| ATOM | 8347 | N | ASN | B | 148 | 29.782 | 75.143 | 27.923 | 1.00 | 45.46 | N |
| ATOM | 8349 | CA | ASN | B | 148 | 29.006 | 74.373 | 27.015 | 1.00 | 41.55 | C |
| ATOM | 8351 | CB | ASN | B | 148 | 29.683 | 74.441 | 25.664 | 1.00 | 43.45 | C |
| ATOM | 8354 | CG | ASN | B | 148 | 28.986 | 73.682 | 24.627 | 1.00 | 41.45 | C |
| ATOM | 8355 | OD1 | ASN | B | 148 | 27.770 | 73.450 | 24.682 | 1.00 | 44.14 | O |
| ATOM | 8356 | ND2 | ASN | B | 148 | 29.744 | 73.296 | 23.627 | 1.00 | 37.99 | N |
| ATOM | 8359 | C | ASN | B | 148 | 28.991 | 72.993 | 27.538 | 1.00 | 43.05 | C |
| ATOM | 8360 | O | ASN | B | 148 | 29.608 | 72.076 | 26.973 | 1.00 | 40.55 | O |
| ATOM | 8361 | N | LEU | B | 149 | 28.283 | 72.847 | 28.648 | 1.00 | 42.80 | N |
| ATOM | 8363 | CA | LEU | B | 149 | 28.132 | 71.521 | 29.273 | 1.00 | 41.70 | C |
| ATOM | 8365 | CB | LEU | B | 149 | 29.275 | 71.263 | 30.278 | 1.00 | 39.84 | C |
| ATOM | 8368 | CG | LEU | B | 149 | 29.338 | 69.945 | 31.031 | 1.00 | 41.12 | C |
| ATOM | 8370 | CD1 | LEU | B | 149 | 30.675 | 69.702 | 31.551 | 1.00 | 39.27 | C |
| ATOM | 8374 | CD2 | LEU | B | 149 | 28.368 | 69.844 | 32.185 | 1.00 | 43.77 | C |
| ATOM | 8378 | C | LEU | B | 149 | 26.812 | 71.556 | 30.000 | 1.00 | 42.18 | C |
| ATOM | 8379 | O | LEU | B | 149 | 26.391 | 72.623 | 30.409 | 1.00 | 48.04 | O |
| ATOM | 8380 | N | PHE | B | 150 | 26.128 | 70.428 | 30.161 | 1.00 | 37.65 | N |
| ATOM | 8382 | CA | PHE | B | 150 | 24.982 | 70.422 | 31.068 | 1.00 | 39.61 | C |
| ATOM | 8384 | CB | PHE | B | 150 | 23.718 | 70.954 | 30.396 | 1.00 | 40.04 | C |
| ATOM | 8387 | CG | PHE | B | 150 | 23.235 | 70.093 | 29.272 | 1.00 | 41.92 | C |
| ATOM | 8388 | CD1 | PHE | B | 150 | 23.411 | 70.479 | 27.963 | 1.00 | 40.21 | C |
| ATOM | 8390 | CE1 | PHE | B | 150 | 22.976 | 69.678 | 26.938 | 1.00 | 37.50 | C |
| ATOM | 8392 | CZ | PHE | B | 150 | 22.352 | 68.486 | 27.193 | 1.00 | 45.37 | C |
| ATOM | 8394 | CE2 | PHE | B | 150 | 22.165 | 68.070 | 28.490 | 1.00 | 40.66 | C |
| ATOM | 8396 | CD2 | PHE | B | 150 | 22.608 | 68.878 | 29.526 | 1.00 | 38.93 | C |
| ATOM | 8398 | C | PHE | B | 150 | 24.829 | 68.982 | 31.584 | 1.00 | 42.19 | C |
| ATOM | 8399 | O | PHE | B | 150 | 25.195 | 68.043 | 30.883 | 1.00 | 41.76 | O |
| ATOM | 8400 | N | SER | B | 151 | 24.321 | 68.798 | 32.803 | 1.00 | 37.28 | N |
| ATOM | 8402 | CA | SER | B | 151 | 24.135 | 67.438 | 33.313 | 1.00 | 42.07 | C |
| ATOM | 8404 | CB | SER | B | 151 | 25.051 | 67.208 | 34.517 | 1.00 | 38.99 | C |
| ATOM | 8407 | OG | SER | B | 151 | 25.053 | 68.364 | 35.324 | 1.00 | 34.21 | O |
| ATOM | 8409 | C | SER | B | 151 | 22.671 | 67.218 | 33.728 | 1.00 | 43.06 | C |
| ATOM | 8410 | O | SER | B | 151 | 21.970 | 68.169 | 34.071 | 1.00 | 45.97 | O |
| ATOM | 8411 | N | LEU | B | 152 | 22.207 | 65.979 | 33.719 | 1.00 | 40.15 | N |
| ATOM | 8413 | CA | LEU | B | 152 | 20.832 | 65.747 | 34.122 | 1.00 | 40.34 | C |
| ATOM | 8415 | CB | LEU | B | 152 | 19.975 | 65.313 | 32.938 | 1.00 | 42.16 | C |
| ATOM | 8418 | CG | LEU | B | 152 | 19.590 | 66.186 | 31.756 | 1.00 | 38.97 | C |
| ATOM | 8420 | CD1 | LEU | B | 152 | 18.685 | 65.352 | 30.911 | 1.00 | 40.87 | C |
| ATOM | 8424 | CD2 | LEU | B | 152 | 18.885 | 67.410 | 32.159 | 1.00 | 38.15 | C |
| ATOM | 8428 | C | LEU | B | 152 | 20.743 | 64.628 | 35.107 | 1.00 | 39.44 | C |
| ATOM | 8429 | O | LEU | B | 152 | 21.228 | 63.573 | 34.843 | 1.00 | 35.13 | O |
| ATOM | 8430 | N | GLN | B | 153 | 20.108 | 64.855 | 36.242 | 1.00 | 43.22 | N |
| ATOM | 8432 | CA | GLN | B | 153 | 19.824 | 63.772 | 37.180 | 1.00 | 42.34 | C |
| ATOM | 8434 | CB | GLN | B | 153 | 20.216 | 64.202 | 38.577 | 1.00 | 41.36 | C |
| ATOM | 8437 | CG | GLN | B | 153 | 20.061 | 63.157 | 39.636 | 1.00 | 43.66 | C |
| ATOM | 8440 | CD | GLN | B | 153 | 19.775 | 63.765 | 41.000 | 1.00 | 46.84 | C |
| ATOM | 8441 | OE1 | GLN | B | 153 | 18.981 | 64.710 | 41.126 | 1.00 | 51.70 | O |
| ATOM | 8442 | NE2 | GLN | B | 153 | 20.437 | 63.238 | 42.022 | 1.00 | 51.83 | N |
| ATOM | 8445 | C | GLN | B | 153 | 18.317 | 63.555 | 37.191 | 1.00 | 44.67 | C |
| ATOM | 8446 | O | GLN | B | 153 | 17.613 | 64.327 | 37.822 | 1.00 | 47.27 | O |
| ATOM | 8447 | N | LEU | B | 154 | 17.803 | 62.538 | 36.510 | 1.00 | 45.13 | N |
| ATOM | 8449 | CA | LEU | B | 154 | 16.364 | 62.281 | 36.551 | 1.00 | 40.93 | C |
| ATOM | 8451 | CB | LEU | B | 154 | 15.907 | 61.599 | 35.270 | 1.00 | 37.85 | C |
| ATOM | 8454 | CG | LEU | B | 154 | 16.262 | 62.308 | 33.959 | 1.00 | 35.17 | C |
| ATOM | 8456 | CD1 | LEU | B | 154 | 15.572 | 61.588 | 32.748 | 1.00 | 32.97 | C |
| ATOM | 8460 | CD2 | LEU | B | 154 | 15.890 | 63.766 | 34.016 | 1.00 | 30.94 | C |
| ATOM | 8464 | C | LEU | B | 154 | 16.081 | 61.330 | 37.707 | 1.00 | 49.47 | C |
| ATOM | 8465 | O | LEU | B | 154 | 16.811 | 60.349 | 37.889 | 1.00 | 54.75 | O |
| ATOM | 8466 | N | CYS | B | 155 | 15.044 | 61.606 | 38.490 | 1.00 | 52.56 | N |
| ATOM | 8468 | CA | CYS | B | 155 | 14.695 | 60.747 | 39.622 | 1.00 | 56.72 | C |
| ATOM | 8470 | CB | CYS | B | 155 | 14.662 | 61.539 | 40.934 | 1.00 | 57.35 | C |
| ATOM | 8473 | SG | CYS | B | 155 | 16.265 | 62.120 | 41.545 | 1.00 | 67.57 | S |
| ATOM | 8474 | C | CYS | B | 155 | 13.353 | 60.028 | 39.473 | 1.00 | 58.59 | C |
| ATOM | 8475 | O | CYS | B | 155 | 12.271 | 60.650 | 39.467 | 1.00 | 57.85 | O |
| ATOM | 8476 | N | GLY | B | 156 | 13.417 | 58.707 | 39.352 | 1.00 | 56.91 | N |
| ATOM | 8478 | CA | GLY | B | 156 | 12.205 | 57.930 | 39.443 | 1.00 | 58.92 | C |
| ATOM | 8481 | C | GLY | B | 156 | 11.860 | 57.937 | 40.923 | 1.00 | 58.63 | C |
| ATOM | 8482 | O | GLY | B | 156 | 12.749 | 58.009 | 41.741 | 1.00 | 56.21 | O |
| ATOM | 8483 | N | ALA | B | 157 | 10.591 | 57.864 | 41.288 | 1.00 | 62.17 | N |
| ATOM | 8485 | CA | ALA | B | 157 | 10.255 | 57.778 | 42.709 | 1.00 | 64.46 | C |
| ATOM | 8487 | CB | ALA | B | 157 | 9.080 | 58.650 | 43.026 | 1.00 | 63.92 | C |
| ATOM | 8491 | C | ALA | B | 157 | 10.022 | 56.338 | 43.211 | 1.00 | 66.10 | C |
| ATOM | 8492 | O | ALA | B | 157 | 9.966 | 56.114 | 44.426 | 1.00 | 64.25 | O |
| ATOM | 8493 | N | GLY | B | 158 | 9.883 | 55.383 | 42.284 | 1.00 | 66.93 | N |
| ATOM | 8495 | CA | GLY | B | 158 | 9.804 | 53.966 | 42.636 | 1.00 | 69.42 | C |
| ATOM | 8498 | C | GLY | B | 158 | 8.530 | 53.537 | 43.351 | 1.00 | 70.66 | C |
| ATOM | 8499 | O | GLY | B | 158 | 8.531 | 52.749 | 44.322 | 1.00 | 69.74 | O |
| ATOM | 8500 | N | PHE | B | 159 | 7.441 | 54.080 | 42.826 | 1.00 | 70.60 | N |
| ATOM | 8502 | CA | PHE | B | 159 | 6.078 | 53.846 | 43.273 | 1.00 | 71.27 | C |
| ATOM | 8504 | CB | PHE | B | 159 | 5.883 | 53.920 | 44.813 | 1.00 | 69.71 | C |
| ATOM | 8507 | CG | PHE | B | 159 | 6.184 | 55.263 | 45.427 | 1.00 | 64.66 | C |
| ATOM | 8508 | CD1 | PHE | B | 159 | 7.228 | 55.397 | 46.334 | 1.00 | 61.90 | C |
| ATOM | 8510 | CE1 | PHE | B | 159 | 7.521 | 56.634 | 46.919 | 1.00 | 65.05 | C |
| ATOM | 8512 | CZ | PHE | B | 159 | 6.751 | 57.756 | 46.599 | 1.00 | 68.53 | C |
| ATOM | 8514 | CE2 | PHE | B | 159 | 5.690 | 57.631 | 45.694 | 1.00 | 67.06 | C |
| ATOM | 8516 | CD2 | PHE | B | 159 | 5.414 | 56.383 | 45.115 | 1.00 | 66.54 | C |
| ATOM | 8518 | C | PHE | B | 159 | 5.311 | 54.914 | 42.502 | 1.00 | 71.04 | C |
| ATOM | 6519 | O | PHE | B | 159 | 5.851 | 55.974 | 42.212 | 1.00 | 68.13 | O |
| ATOM | 8520 | N | PRO | B | 160 | 4.064 | 54.657 | 42.157 | 1.00 | 71.26 | N |
| ATOM | 8521 | CA | PRO | B | 160 | 3.361 | 55.586 | 41.283 | 1.00 | 72.10 | C |
| ATOM | 8523 | CB | PRO | B | 160 | 2.102 | 54.812 | 40.899 | 1.00 | 72.89 | C |
| ATOM | 8526 | CG | PRO | B | 160 | 2.325 | 53.429 | 41.341 | 1.00 | 71.93 | C |
| ATOM | 8529 | CD | PRO | B | 160 | 3.218 | 53.519 | 42.533 | 1.00 | 69.92 | C |
| ATOM | 8532 | C | PRO | B | 160 | 2.959 | 56.894 | 41.919 | 1.00 | 71.65 | C |
| ATOM | 8533 | O | PRO | B | 160 | 2.888 | 57.045 | 43.136 | 1.00 | 72.02 | O |
| ATOM | 8534 | N | LEU | B | 161 | 2.703 | 57.841 | 41.039 | 1.00 | 71.52 | N |
| ATOM | 8536 | CA | LEU | B | 161 | 2.108 | 59.104 | 41.403 | 1.00 | 75.59 | C |
| ATOM | 8538 | CB | LEU | B | 161 | 3.160 | 60.196 | 41.575 | 1.00 | 77.46 | C |
| ATOM | 8541 | CG | LEU | B | 161 | 4.493 | 59.938 | 42.269 | 1.00 | 78.23 | C |
| ATOM | 8543 | CD1 | LEU | B | 161 | 5.608 | 60.398 | 41.338 | 1.00 | 78.97 | C |
| ATOM | 8547 | CD2 | LEU | B | 161 | 4.570 | 60.663 | 43.611 | 1.00 | 75.00 | C |
| ATOM | 8551 | C | LEU | B | 161 | 1.177 | 59.500 | 40.252 | 1.00 | 76.43 | C |
| ATOM | 8552 | O | LEU | B | 161 | 1.492 | 59.322 | 39.071 | 1.00 | 79.99 | O |
| ATOM | 8553 | N | GLN | B | 162 | 0.016 | 60.022 | 40.584 | 1.00 | 75.26 | N |
| ATOM | 8555 | CA | GLN | B | 162 | -0.853 | 60.579 | 39.563 | 1.00 | 75.96 | C |
| ATOM | 8557 | CB | GLN | B | 162 | -2.266 | 60.698 | 40.126 | 1.00 | 75.84 | C |
| ATOM | 8560 | CG | GLN | B | 162 | -2.314 | 61.141 | 41.592 | 1.00 | 72.01 | C |
| ATOM | 8563 | CD | GLN | B | 162 | -1.628 | 62.471 | 41.822 | 1.00 | 71.95 | C |
| ATOM | 8564 | OE1 | GLN | B | 162 | -0.588 | 62.767 | 41.223 | 1.00 | 69.09 | O |
| ATOM | 8565 | NE2 | GLN | B | 162 | -2.203 | 63.281 | 42.696 | 1.00 | 72.77 | N |
| ATOM | 8568 | C | GLN | B | 162 | -0.290 | 61.961 | 39.102 | 1.00 | 77.71 | C |
| ATOM | 8569 | O | GLN | B | 162 | 0.933 | 62.153 | 39.019 | 1.00 | 79.30 | O |
| ATOM | 8570 | N | GLN | B | 163 | -1.163 | 62.919 | 38.811 | 1.00 | 74.89 | N |
| ATOM | 8572 | CA | GLN | B | 163 | -0.706 | 64.215 | 38.347 | 1.00 | 76.31 | C |
| ATOM | 8574 | CB | GLN | B | 163 | -1.665 | 64.799 | 37.275 | 1.00 | 74.58 | C |
| ATOM | 8577 | CG | GLN | B | 163 | -1.457 | 66.276 | 36.955 | 1.00 | 75.98 | C |
| ATOM | 8580 | CD | GLN | B | 163 | -1.774 | 66.679 | 35.491 | 1.00 | 78.50 | C |
| ATOM | 8581 | OE1 | GLN | B | 163 | -2.869 | 66.428 | 34.982 | 1.00 | 73.62 | O |
| ATOM | 8582 | NE2 | GLN | B | 163 | -0.797 | 67.316 | 34.828 | 1.00 | 77.14 | N |
| ATOM | 8585 | C | GLN | B | 163 | -0.497 | 65.139 | 39.566 | 1.00 | 78.42 | C |
| ATOM | 8586 | O | GLN | B | 163 | 0.544 | 65.807 | 39.667 | 1.00 | 84.37 | O |
| ATOM | 8587 | N | SER | B | 164 | -1.452 | 65.154 | 40.501 | 1.00 | 76.38 | N |
| ATOM | 8589 | CA | SER | B | 164 | -1.369 | 66.023 | 41.690 | 1.00 | 73.78 | C |
| ATOM | 8591 | CB | SER | B | 164 | -2.442 | 65.635 | 42.714 | 1.00 | 71.78 | C |
| ATOM | 8594 | OG | SER | B | 164 | -2.807 | 66.743 | 43.533 | 1.00 | 71.79 | O |
| ATOM | 8596 | C | SER | B | 164 | -0.014 | 65.965 | 42.395 | 1.00 | 76.38 | C |
| ATOM | 8597 | O | SER | B | 164 | 0.467 | 66.969 | 42.942 | 1.00 | 71.19 | O |
| ATOM | 8598 | N | GLU | B | 165 | 0.590 | 64.778 | 42.378 | 1.00 | 78.65 | N |
| ATOM | 8600 | CA | GLU | B | 165 | 1.805 | 64.521 | 43.125 | 1.00 | 82.23 | C |
| ATOM | 8602 | CB | GLU | B | 165 | 1.948 | 63.017 | 43.344 | 1.00 | 86.01 | C |
| ATOM | 8605 | CG | GLU | B | 165 | 0.848 | 62.399 | 44.201 | 1.00 | 88.14 | C |
| ATOM | 8608 | CD | GLU | B | 165 | 1.063 | 60.911 | 44.445 | 1.00 | 90.26 | C |
| ATOM | 8609 | OE1 | GLU | B | 165 | 2.025 | 60.560 | 45.159 | 1.00 | 87.30 | O |
| ATOM | 8610 | OE2 | GLU | B | 165 | 0.266 | 60.089 | 43.928 | 1.00 | 95.66 | O |
| ATOM | 8611 | C | GLU | B | 165 | 3.053 | 65.082 | 42.436 | 1.00 | 81.15 | C |
| ATOM | 8612 | O | GLU | B | 165 | 3.975 | 65.580 | 43.096 | 1.00 | 76.40 | O |
| ATOM | 8613 | N | VAL | B | 166 | 3.080 | 64.989 | 41.111 | 1.00 | 79.46 | N |
| ATOM | 8615 | CA | VAL | B | 166 | 4.196 | 65.532 | 40.338 | 1.00 | 77.43 | C |
| ATOM | 8617 | CB | VAL | B | 166 | 4.063 | 65.124 | 38.829 | 1.00 | 75.19 | C |
| ATOM | 8619 | CG1 | VAL | B | 166 | 4.836 | 66.023 | 37.922 | 1.00 | 77.04 | C |
| ATOM | 8623 | CG2 | VAL | B | 166 | 4.538 | 63.703 | 38.634 | 1.00 | 74.61 | C |
| ATOM | 8627 | C | VAL | B | 166 | 4.353 | 67.060 | 40.568 | 1.00 | 75.47 | C |
| ATOM | 8628 | O | VAL | B | 166 | 5.468 | 67.551 | 40.621 | 1.00 | 75.66 | O |
| ATOM | 8629 | N | LEU | B | 167 | 3.259 | 67.804 | 40.727 | 1.00 | 75.60 | N |
| ATOM | 8631 | CA | LEU | B | 167 | 3.348 | 69.261 | 40.931 | 1.00 | 75.68 | C |
| ATOM | 8633 | CB | LEU | B | 167 | 1.977 | 69.929 | 40.772 | 1.00 | 76.77 | C |
| ATOM | 8636 | CG | LEU | B | 167 | 1.248 | 69.774 | 39.430 | 1.00 | 80.17 | C |
| ATOM | 8638 | CD1 | LEU | B | 167 | −0.170 | 70.365 | 39.474 | 1.00 | 77.26 | C |
| ATOM | 8642 | CD2 | LEU | B | 167 | 2.047 | 70.399 | 38.282 | 1.00 | 82.87 | C |
| ATOM | 8646 | C | LEU | B | 167 | 3.887 | 69.644 | 42.302 | 1.00 | 73.27 | C |
| ATOM | 8647 | O | LEU | B | 167 | 4.593 | 70.643 | 42.476 | 1.00 | 69.74 | O |
| ATOM | 8648 | N | ALA | B | 168 | 3.543 | 68.848 | 43.288 | 1.00 | 73.73 | N |
| ATOM | 8650 | CA | ALA | B | 168 | 3.962 | 69.147 | 44.636 | 1.00 | 77.27 | C |
| ATOM | 8652 | CB | ALA | B | 168 | 2.949 | 68.585 | 45.638 | 1.00 | 75.98 | C |
| ATOM | 8656 | C | ALA | B | 168 | 5.353 | 68.596 | 44.921 | 1.00 | 78.89 | C |
| ATOM | 8657 | O | ALA | B | 168 | 5.995 | 69.005 | 45.889 | 1.00 | 84.58 | O |
| ATOM | 8658 | N | SER | B | 169 | 5.832 | 67.688 | 44.083 | 1.00 | 76.97 | N |
| ATOM | 8660 | CA | SER | B | 169 | 7.094 | 67.029 | 44.373 | 1.00 | 77.52 | C |
| ATOM | 8662 | CB | SER | B | 169 | 6.858 | 65.549 | 44.637 | 1.00 | 80.08 | C |
| ATOM | 8665 | OG | SER | B | 169 | 5.612 | 65.396 | 45.312 | 1.00 | 83.47 | O |
| ATOM | 8667 | C | SER | B | 169 | 8.122 | 67.204 | 43.285 | 1.00 | 75.54 | C |
| ATOM | 8668 | O | SER | B | 169 | 7.817 | 67.604 | 42.162 | 1.00 | 69.75 | O |
| ATOM | 8669 | N | VAL | B | 170 | 9.355 | 66.890 | 43.658 | 1.00 | 74.47 | N |
| ATOM | 8671 | CA | VAL | B | 170 | 10.511 | 67.097 | 42.805 | 1.00 | 73.73 | C |
| ATOM | 8673 | CB | VAL | B | 170 | 11.777 | 67.421 | 43.675 | 1.00 | 72.68 | C |
| ATOM | 8675 | CG1 | VAL | B | 170 | 13.078 | 67.140 | 42.959 | 1.00 | 74.83 | C |
| ATOM | 8679 | CG2 | VAL | B | 170 | 11.755 | 68.881 | 44.098 | 1.00 | 72.94 | C |
| ATOM | 8683 | C | VAL | B | 170 | 10.654 | 65.893 | 41.861 | 1.00 | 71.65 | C |
| ATOM | 8684 | O | VAL | B | 170 | 9.950 | 64.866 | 42.012 | 1.00 | 75.35 | O |
| ATOM | 8685 | N | GLY | B | 171 | 11.525 | 66.035 | 40.863 | 1.00 | 64.10 | N |
| ATOM | 8687 | CA | GLY | B | 171 | 11.730 | 64.987 | 39.890 | 1.00 | 57.27 | C |
| ATOM | 8690 | C | GLY | B | 171 | 13.146 | 64.918 | 39.419 | 1.00 | 52.94 | C |
| ATOM | 8691 | O | GLY | B | 171 | 13.434 | 64.116 | 38.563 | 1.00 | 50.16 | O |
| ATOM | 8692 | N | GLY | B | 172 | 14.024 | 65.757 | 39.959 | 1.00 | 48.11 | N |
| ATOM | 8694 | CA | GLY | B | 172 | 15.412 | 65.704 | 39.591 | 1.00 | 48.37 | C |
| ATOM | 8697 | C | GLY | B | 172 | 15.975 | 67.066 | 39.249 | 1.00 | 51.80 | C |
| ATOM | 8698 | O | GLY | B | 172 | 15.357 | 68.102 | 39.524 | 1.00 | 54.56 | O |
| ATOM | 8699 | N | SER | B | 173 | 17.167 | 67.063 | 38.658 | 1.00 | 48.83 | N |
| ATOM | 8701 | CA | SER | B | 173 | 17.796 | 68.288 | 38.259 | 1.00 | 43.89 | C |
| ATOM | 8703 | CB | SER | B | 173 | 19.000 | 68.557 | 39.113 | 1.00 | 42.65 | C |
| ATOM | 8706 | OG | SER | B | 173 | 18.603 | 69.033 | 40.373 | 1.00 | 49.32 | O |
| ATOM | 8708 | C | SER | B | 173 | 18.297 | 68.264 | 36.864 | 1.00 | 44.21 | C |
| ATOM | 8709 | O | SER | B | 173 | 18.544 | 67.207 | 36.273 | 1.00 | 45.21 | O |
| ATOM | 8710 | N | MET | B | 174 | 18.432 | 69.476 | 36.356 | 1.00 | 43.20 | N |
| ATOM | 8712 | CA | MET | B | 174 | 19.138 | 69.725 | 35.148 | 1.00 | 43.58 | C |
| ATOM | 8714 | CB | MET | B | 174 | 18.184 | 70.144 | 34.044 | 1.00 | 43.84 | C |
| ATOM | 8717 | CG | MET | B | 174 | 18.971 | 70.650 | 32.883 | 1.00 | 46.91 | C |
| ATOM | 8720 | SD | MET | B | 174 | 18.074 | 70.998 | 31.472 | 1.00 | 53.60 | S |
| ATOM | 8721 | CE | MET | B | 174 | 17.365 | 72.553 | 31.872 | 1.00 | 56.75 | C |
| ATOM | 8725 | C | MET | B | 174 | 20.132 | 70.872 | 35.471 | 1.00 | 45.69 | C |
| ATOM | 8726 | O | MET | B | 174 | 19.721 | 72.028 | 35.575 | 1.00 | 45.35 | O |
| ATOM | 8727 | N | ILE | B | 175 | 21.418 | 70.577 | 35.650 | 1.00 | 41.84 | N |
| ATOM | 8729 | CA | ILE | B | 175 | 22.376 | 71.663 | 35.853 | 1.00 | 46.48 | C |
| ATOM | 8731 | CB | ILE | B | 175 | 23.548 | 71.295 | 36.811 | 1.00 | 47.95 | C |
| ATOM | 8733 | CG1 | ILE | B | 175 | 23.105 | 70.444 | 37.989 | 1.00 | 46.56 | C |
| ATOM | 8736 | CD1 | ILE | B | 175 | 22.250 | 71.153 | 38.942 | 1.00 | 46.76 | C |
| ATOM | 8740 | CG2 | ILE | B | 175 | 24.168 | 72.567 | 37.289 | 1.00 | 45.43 | C |
| ATOM | 8744 | C | ILE | B | 175 | 22.986 | 72.113 | 34.524 | 1.00 | 44.88 | C |
| ATOM | 8745 | O | ILE | B | 175 | 23.615 | 71.333 | 33.802 | 1.00 | 48.63 | O |
| ATOM | 8746 | N | ILE | B | 176 | 22.815 | 73.385 | 34.216 | 1.00 | 51.15 | N |
| ATOM | 8748 | CA | ILE | B | 176 | 23.223 | 73.942 | 32.922 | 1.00 | 51.24 | C |
| ATOM | 8750 | CB | ILE | B | 176 | 22.198 | 74.980 | 32.470 | 1.00 | 51.72 | C |
| ATOM | 8752 | CG1 | ILE | B | 176 | 20.833 | 74.369 | 32.300 | 1.00 | 45.30 | C |
| ATOM | 8755 | CD1 | ILE | B | 176 | 20.454 | 74.362 | 30.860 | 1.00 | 49.64 | C |
| ATOM | 8759 | CG2 | ILE | B | 176 | 22.621 | 75.606 | 31.135 | 1.00 | 53.65 | C |
| ATOM | 8763 | C | ILE | B | 176 | 24.558 | 74.647 | 33.020 | 1.00 | 50.93 | C |
| ATOM | 8764 | O | ILE | B | 176 | 24.648 | 75.704 | 33.620 | 1.00 | 52.76 | O |
| ATOM | 8765 | N | GLY | B | 177 | 25.584 | 74.065 | 32.413 | 1.00 | 53.67 | N |
| ATOM | 8767 | CA | GLY | B | 177 | 26.921 | 74.625 | 32.462 | 1.00 | 52.15 | C |
| ATOM | 8770 | C | GLY | B | 177 | 27.839 | 74.139 | 33.561 | 1.00 | 48.67 | C |
| ATOM | 8771 | O | GLY | B | 177 | 28.832 | 74.776 | 33.795 | 1.00 | 52.76 | O |
| ATOM | 8772 | N | GLY | B | 178 | 27.538 | 73.029 | 34.224 | 1.00 | 51.24 | N |
| ATOM | 8774 | CA | GLY | B | 178 | 28.406 | 72.536 | 35.293 | 1.00 | 51.26 | C |
| ATOM | 8777 | C | GLY | B | 178 | 28.016 | 71.184 | 35.886 | 1.00 | 52.43 | C |
| ATOM | 8778 | O | GLY | B | 178 | 27.068 | 70.577 | 35.440 | 1.00 | 59.00 | O |
| ATOM | 8779 | N | ILE | B | 179 | 28.730 | 70.717 | 36.903 | 1.00 | 50.07 | N |
| ATOM | 8781 | CA | ILE | B | 179 | 28.446 | 69.433 | 37.512 | 1.00 | 45.67 | C |
| ATOM | 8783 | CB | ILE | B | 179 | 29.610 | 68.519 | 37.252 | 1.00 | 47.32 | C |
| ATOM | 8785 | CG1 | ILE | B | 179 | 29.842 | 68.357 | 35.761 | 1.00 | 49.07 | C |
| ATOM | 8788 | CD1 | ILE | B | 179 | 31.133 | 67.633 | 35.429 | 1.00 | 45.35 | C |
| ATOM | 8792 | CG2 | ILE | B | 179 | 29.350 | 67.174 | 37.842 | 1.00 | 48.87 | C |
| ATOM | 8796 | C | ILE | B | 179 | 28.271 | 69.601 | 39.024 | 1.00 | 46.31 | C |
| ATOM | 8797 | O | ILE | B | 179 | 29.115 | 70.203 | 39.691 | 1.00 | 48.40 | O |
| ATOM | 8798 | N | ASP | B | 180 | 27.199 | 69.060 | 39.581 | 1.00 | 45.06 | N |
| ATOM | 8800 | CA | ASP | B | 180 | 26.925 | 69.265 | 40.996 | 1.00 | 46.20 | C |
| ATOM | 8802 | CB | ASP | B | 180 | 25.489 | 69.733 | 41.193 | 1.00 | 48.62 | C |
| ATOM | 8805 | CG | ASP | B | 180 | 25.183 | 70.143 | 42.644 | 1.00 | 50.85 | C |
| ATOM | 8806 | OD1 | ASP | B | 180 | 25.059 | 71.353 | 42.922 | 1.00 | 46.55 | O |
| ATOM | 8807 | OD2 | ASP | B | 180 | 25.034 | 69.324 | 43.579 | 1.00 | 57.84 | O |
| ATOM | 8808 | C | ASP | B | 180 | 27.169 | 68.037 | 41.828 | 1.00 | 46.31 | C |
| ATOM | 8809 | O | ASP | B | 180 | 26.308 | 67.217 | 41.996 | 1.00 | 52.16 | O |
| ATOM | 8810 | N | HIS | B | 181 | 28.377 | 67.942 | 42.351 | 1.00 | 51.89 | N |
| ATOM | 8812 | CA | HIS | B | 181 | 28.831 | 66.897 | 43.277 | 1.00 | 50.52 | C |
| ATOM | 8814 | CB | HIS | B | 181 | 29.990 | 67.496 | 44.061 | 1.00 | 50.18 | C |
| ATOM | 8817 | CG | HIS | B | 181 | 31.190 | 67.731 | 43.220 | 1.00 | 60.85 | C |
| ATOM | 8818 | ND1 | HIS | B | 181 | 32.309 | 68.400 | 43.673 | 1.00 | 67.21 | N |
| ATOM | 8820 | CE1 | HIS | B | 181 | 33.207 | 68.450 | 42.704 | 1.00 | 70.69 | C |
| ATOM | 8822 | NE2 | HIS | B | 181 | 32.708 | 67.841 | 41.639 | 1.00 | 75.54 | N |
| ATOM | 8824 | CD2 | HIS | B | 181 | 31.447 | 67.381 | 41.936 | 1.00 | 67.45 | C |
| ATOM | 8826 | C | HIS | B | 181 | 27.858 | 66.226 | 44.276 | 1.00 | 48.76 | C |
| ATOM | 8827 | O | HIS | B | 181 | 28.090 | 65.086 | 44.671 | 1.00 | 50.74 | O |
| ATOM | 8828 | N | SER | B | 182 | 26.797 | 66.891 | 44.718 | 1.00 | 47.39 | N |
| ATOM | 8830 | CA | SER | B | 182 | 25.899 | 66.216 | 45.653 | 1.00 | 47.06 | C |
| ATOM | 8832 | CB | SER | B | 182 | 25.005 | 67.213 | 46.359 | 1.00 | 44.50 | C |
| ATOM | 8835 | OG | SER | B | 182 | 24.325 | 67.978 | 45.398 | 1.00 | 49.42 | O |
| ATOM | 8837 | C | SER | B | 182 | 25.041 | 65.202 | 44.909 | 1.00 | 44.21 | C |
| ATOM | 8838 | O | SER | B | 182 | 24.473 | 64.259 | 45.490 | 1.00 | 42.19 | O |
| ATOM | 8839 | N | LEU | B | 183 | 24.941 | 65.377 | 43.611 | 1.00 | 35.22 | N |
| ATOM | 8841 | CA | LEU | B | 183 | 24.017 | 64.548 | 42.910 | 1.00 | 34.72 | C |
| ATOM | 8843 | CB | LEU | B | 183 | 23.541 | 65.242 | 41.625 | 1.00 | 37.22 | C |
| ATOM | 8846 | CG | LEU | B | 183 | 23.060 | 66.693 | 41.814 | 1.00 | 35.27 | C |
| ATOM | 8848 | CD1 | LEU | B | 183 | 22.723 | 67.333 | 40.490 | 1.00 | 35.13 | C |
| ATOM | 8852 | CD2 | LEU | B | 183 | 21.845 | 66.772 | 42.757 | 1.00 | 39.21 | C |
| ATOM | 8856 | C | LEU | B | 183 | 24.581 | 63.170 | 42.659 | 1.00 | 34.54 | C |
| ATOM | 8857 | O | LEU | B | 183 | 23.893 | 62.348 | 42.140 | 1.00 | 32.68 | O |
| ATOM | 8858 | N | TYR | B | 184 | 25.814 | 62.867 | 43.032 | 1.00 | 40.53 | N |
| ATOM | 8860 | CA | TYR | B | 184 | 26.304 | 61.495 | 42.751 | 1.00 | 40.17 | C |
| ATOM | 8862 | CB | TYR | B | 184 | 26.806 | 61.441 | 41.320 | 1.00 | 42.80 | C |
| ATOM | 8865 | CG | TYR | B | 184 | 28.109 | 62.182 | 41.128 | 1.00 | 39.74 | C |
| ATOM | 8866 | CD1 | TYR | B | 184 | 28.111 | 63.548 | 40.992 | 1.00 | 37.66 | C |
| ATOM | 8868 | CE1 | TYR | B | 184 | 29.253 | 64.256 | 40.803 | 1.00 | 37.61 | C |
| ATOM | 8870 | CZ | TYR | B | 184 | 30.456 | 63.621 | 40.763 | 1.00 | 47.41 | C |
| ATOM | 8871 | OH | TYR | B | 184 | 31.576 | 64.426 | 40.581 | 1.00 | 55.00 | O |
| ATOM | 8873 | CE2 | TYR | B | 184 | 30.520 | 62.236 | 40.906 | 1.00 | 43.25 | C |
| ATOM | 8875 | CD2 | TYR | B | 184 | 29.322 | 61.520 | 41.083 | 1.00 | 41.71 | C |
| ATOM | 8877 | C | TYR | B | 184 | 27.436 | 60.952 | 43.634 | 1.00 | 41.79 | C |
| ATOM | 8878 | O | TYR | B | 184 | 28.095 | 61.684 | 44.365 | 1.00 | 46.68 | O |
| ATOM | 8879 | N | THR | B | 185 | 27.686 | 59.655 | 43.536 | 1.00 | 43.21 | N |
| ATOM | 8881 | CA | THR | B | 185 | 28.744 | 59.045 | 44.328 | 1.00 | 44.21 | C |
| ATOM | 8883 | CB | THR | B | 185 | 28.163 | 58.164 | 45.366 | 1.00 | 44.37 | C |
| ATOM | 8885 | OG1 | THR | B | 185 | 27.282 | 57.235 | 44.714 | 1.00 | 55.13 | O |
| ATOM | 8887 | CG2 | THR | B | 185 | 27.257 | 58.969 | 46.275 | 1.00 | 43.63 | C |
| ATOM | 8891 | C | THR | B | 185 | 29.676 | 58.215 | 43.499 | 1.00 | 43.25 | C |
| ATOM | 8892 | O | THR | B | 185 | 29.280 | 57.701 | 42.428 | 1.00 | 41.22 | O |
| ATOM | 8893 | N | GLY | B | 186 | 30.912 | 58.084 | 44.009 | 1.00 | 36.99 | N |
| ATOM | 8895 | CA | GLY | B | 186 | 31.934 | 57.353 | 43.311 | 1.00 | 37.15 | C |
| ATOM | 8898 | C | GLY | B | 186 | 32.359 | 58.174 | 42.111 | 1.00 | 39.52 | C |
| ATOM | 8899 | O | GLY | B | 186 | 31.993 | 59.309 | 41.970 | 1.00 | 41.98 | O |
| ATOM | 8900 | N | SER | B | 187 | 33.106 | 57.606 | 41.199 | 1.00 | 41.91 | N |
| ATOM | 8902 | CA | SER | B | 187 | 33.702 | 58.455 | 40.213 | 1.00 | 43.54 | C |
| ATOM | 8904 | CB | SER | B | 187 | 35.132 | 57.957 | 39.931 | 1.00 | 49.17 | C |
| ATOM | 8907 | OG | SER | B | 187 | 35.905 | 57.864 | 41.143 | 1.00 | 51.70 | O |
| ATOM | 8909 | C | SER | B | 187 | 32.906 | 58.552 | 38.968 | 1.00 | 38.74 | C |
| ATOM | 8910 | O | SER | B | 187 | 32.171 | 57.665 | 38.677 | 1.00 | 44.27 | O |
| ATOM | 8911 | N | LEU | B | 188 | 33.054 | 59.649 | 38.236 | 1.00 | 36.60 | N |
| ATOM | 8913 | CA | LEU | B | 188 | 32.451 | 59.779 | 36.908 | 1.00 | 36.77 | C |
| ATOM | 8915 | CB | LEU | B | 188 | 32.452 | 61.247 | 36.480 | 1.00 | 29.29 | C |
| ATOM | 8918 | CG | LEU | B | 188 | 31.170 | 62.059 | 36.695 | 1.00 | 26.22 | C |
| ATOM | 8920 | CD1 | LEU | B | 188 | 31.400 | 63.517 | 36.419 | 1.00 | 31.52 | C |
| ATOM | 8924 | CD2 | LEU | B | 188 | 30.078 | 61.647 | 35.819 | 1.00 | 28.00 | C |
| ATOM | 8928 | C | LEU | B | 188 | 33.233 | 59.037 | 35.839 | 1.00 | 37.01 | C |
| ATOM | 8929 | O | LEU | B | 188 | 34.379 | 59.346 | 35.689 | 1.00 | 42.16 | O |
| ATOM | 8930 | N | TRP | B | 189 | 32.622 | 58.095 | 35.096 | 1.00 | 44.14 | N |
| ATOM | 8932 | CA | TRP | B | 189 | 33.225 | 57.450 | 33.888 | 1.00 | 40.20 | C |
| ATOM | 8934 | CB | TRP | B | 189 | 33.043 | 55.957 | 33.873 | 1.00 | 42.63 | C |
| ATOM | 8937 | CG | TRP | B | 189 | 33.929 | 55.247 | 34.839 | 1.00 | 44.06 | C |
| ATOM | 8938 | CD1 | TRP | B | 189 | 33.638 | 54.972 | 36.099 | 1.00 | 38.39 | C |
| ATOM | 8940 | NE1 | TRP | B | 189 | 34.685 | 54.319 | 36.695 | 1.00 | 45.18 | N |
| ATOM | 8942 | CE2 | TRP | B | 189 | 35.687 | 54.158 | 35.787 | 1.00 | 44.17 | C |
| ATOM | 8943 | CD2 | TRP | B | 189 | 35.248 | 54.733 | 34.600 | 1.00 | 44.91 | C |
| ATOM | 8944 | CE3 | TRP | B | 189 | 36.102 | 54.699 | 33.487 | 1.00 | 53.36 | C |
| ATOM | 8946 | CZ3 | TRP | B | 189 | 37.351 | 54.090 | 33.618 | 1.00 | 52.20 | C |
| ATOM | 8948 | CH2 | TRP | B | 189 | 37.752 | 53.528 | 34.833 | 1.00 | 50.78 | C |
| ATOM | 8950 | CZ2 | TRP | B | 189 | 36.938 | 53.549 | 35.923 | 1.00 | 50.20 | C |
| ATOM | 8952 | C | TRP | B | 189 | 32.610 | 57.975 | 32.572 | 1.00 | 43.79 | C |
| ATOM | 8953 | O | TRP | B | 189 | 31.391 | 58.184 | 32.480 | 1.00 | 46.26 | O |
| ATOM | 8954 | N | TYR | B | 190 | 33.469 | 58.151 | 31.560 | 1.00 | 42.68 | N |
| ATOM | 8956 | CA | TYR | B | 190 | 33.154 | 58.828 | 30.296 | 1.00 | 39.84 | C |
| ATOM | 8958 | CB | TYR | B | 190 | 34.052 | 60.070 | 30.215 | 1.00 | 36.42 | C |
| ATOM | 8961 | CG | TYR | B | 190 | 33.685 | 61.270 | 31.080 | 1.00 | 33.81 | C |
| ATOM | 8962 | CD1 | TYR | B | 190 | 34.442 | 61.624 | 32.172 | 1.00 | 37.07 | C |
| ATOM | 8964 | CE1 | TYR | B | 190 | 34.111 | 62.703 | 32.935 | 1.00 | 42.72 | C |
| ATOM | 8966 | CZ | TYR | B | 190 | 32.992 | 63.450 | 32.590 | 1.00 | 45.10 | C |
| ATOM | 8967 | OH | TYR | B | 190 | 32.601 | 64.565 | 33.313 | 1.00 | 53.55 | O |
| ATOM | 8969 | CE2 | TYR | B | 190 | 32.255 | 63.102 | 31.516 | 1.00 | 30.96 | C |
| ATOM | 8971 | CD2 | TYR | B | 190 | 32.597 | 62.043 | 30.775 | 1.00 | 33.25 | C |
| ATOM | 8973 | C | TYR | B | 190 | 33.386 | 58.066 | 28.964 | 1.00 | 38.96 | C |
| ATOM | 8974 | O | TYR | B | 190 | 34.524 | 57.748 | 28.621 | 1.00 | 51.45 | O |
| ATOM | 8975 | N | THR | B | 191 | 32.312 | 57.822 | 28.210 | 1.00 | 47.13 | N |
| ATOM | 8977 | CA | THR | B | 191 | 32.398 | 57.242 | 26.864 | 1.00 | 47.27 | C |
| ATOM | 8979 | CB | THR | B | 191 | 31.143 | 56.433 | 26.566 | 1.00 | 47.24 | C |
| ATOM | 8981 | OG1 | THR | B | 191 | 31.383 | 55.461 | 25.550 | 1.00 | 50.31 | O |
| ATOM | 8983 | CG2 | THR | B | 191 | 30.100 | 57.286 | 25.966 | 1.00 | 48.46 | C |
| ATOM | 8987 | C | THR | B | 191 | 32.464 | 58.404 | 25.872 | 1.00 | 51.71 | C |
| ATOM | 8988 | O | THR | B | 191 | 31.924 | 59.462 | 26.120 | 1.00 | 56.58 | O |
| ATOM | 8989 | N | PRO | B | 192 | 33.116 | 58.236 | 24.741 | 1.00 | 54.66 | N |
| ATOM | 8990 | CA | PRO | B | 192 | 33.183 | 59.339 | 23.783 | 1.00 | 53.51 | C |
| ATOM | 8992 | CB | PRO | B | 192 | 34.249 | 58.903 | 22.788 | 1.00 | 53.85 | C |
| ATOM | 8995 | CG | PRO | B | 192 | 34.716 | 57.512 | 23.244 | 1.00 | 56.15 | C |
| ATOM | 8998 | CD | PRO | B | 192 | 33.802 | 57.022 | 24.282 | 1.00 | 54.11 | C |
| ATOM | 9001 | C | PRO | B | 192 | 31.875 | 59.435 | 23.058 | 1.00 | 53.29 | C |
| ATOM | 9002 | O | PRO | B | 192 | 31.105 | 58.500 | 23.175 | 1.00 | 48.91 | O |
| ATOM | 9003 | N | ILE | B | 193 | 31.654 | 60.547 | 22.351 | 1.00 | 53.07 | N |
| ATOM | 9005 | CA | ILE | B | 193 | 30.506 | 60.753 | 21.490 | 1.00 | 47.51 | C |
| ATOM | 9007 | CB | ILE | B | 193 | 29.967 | 62.149 | 21.619 | 1.00 | 53.03 | C |
| ATOM | 9009 | CG1 | ILE | B | 193 | 29.103 | 62.310 | 22.871 | 1.00 | 53.04 | C |
| ATOM | 9012 | CD1 | ILE | B | 193 | 28.952 | 63.742 | 23.319 | 1.00 | 53.15 | C |
| ATOM | 9016 | CG2 | ILE | B | 193 | 29.109 | 62.457 | 20.421 | 1.00 | 53.72 | C |
| ATOM | 9020 | C | ILE | B | 193 | 31.029 | 60.603 | 20.091 | 1.00 | 50.01 | C |
| ATOM | 9021 | O | ILE | B | 193 | 31.857 | 61.413 | 19.615 | 1.00 | 45.74 | O |
| ATOM | 9022 | N | ARG | B | 194 | 30.519 | 59.566 | 19.442 | 1.00 | 49.20 | N |
| ATOM | 9024 | CA | ARG | B | 194 | 30.954 | 59.093 | 18.139 | 1.00 | 48.45 | C |
| ATOM | 9026 | CB | ARG | B | 194 | 30.161 | 57.826 | 17.861 | 1.00 | 46.41 | C |
| ATOM | 9029 | CG | ARG | B | 194 | 30.797 | 56.851 | 16.901 | 1.00 | 47.20 | C |
| ATOM | 9032 | CD | ARG | B | 194 | 29.735 | 56.067 | 16.153 | 1.00 | 43.61 | C |
| ATOM | 9035 | NE | ARG | B | 194 | 30.326 | 55.148 | 15.205 | 1.00 | 44.33 | N |
| ATOM | 9037 | CZ | ARG | B | 194 | 29.624 | 54.477 | 14.301 | 1.00 | 45.08 | C |
| ATOM | 9038 | NH1 | ARG | B | 194 | 28.331 | 54.639 | 14.241 | 1.00 | 42.10 | N |
| ATOM | 9041 | NH2 | ARG | B | 194 | 30.207 | 53.647 | 13.452 | 1.00 | 44.38 | N |
| ATOM | 9044 | C | ARG | B | 194 | 30.740 | 60.071 | 16.978 | 1.00 | 55.22 | C |
| ATOM | 9045 | O | ARG | B | 194 | 31.503 | 60.047 | 15.987 | 1.00 | 56.72 | O |
| ATOM | 9046 | N | ARG | B | 195 | 29.696 | 60.906 | 17.113 | 1.00 | 57.00 | N |
| ATOM | 9048 | CA | ARG | B | 195 | 29.244 | 61.865 | 16.087 | 1.00 | 53.97 | C |
| ATOM | 9050 | CB | ARG | B | 195 | 28.412 | 61.171 | 15.017 | 1.00 | 55.76 | C |
| ATOM | 9053 | CG | ARG | B | 195 | 28.550 | 61.762 | 13.630 | 1.00 | 60.64 | C |
| ATOM | 9056 | CD | ARG | B | 195 | 27.234 | 61.923 | 12.849 | 1.00 | 60.09 | C |
| ATOM | 9059 | NE | ARG | B | 195 | 26.847 | 60.696 | 12.164 | 1.00 | 64.82 | N |
| ATOM | 9061 | CZ | ARG | B | 195 | 26.074 | 60.632 | 11.079 | 1.00 | 66.70 | C |
| ATOM | 9062 | NH1 | ARG | B | 195 | 25.572 | 61.735 | 10.520 | 1.00 | 66.60 | N |
| ATOM | 9065 | NH2 | ARG | B | 195 | 25.799 | 59.444 | 10.549 | 1.00 | 67.97 | N |
| ATOM | 9068 | C | ARG | B | 195 | 28.326 | 62.857 | 16.769 | 1.00 | 52.68 | C |
| ATOM | 9069 | O | ARG | B | 195 | 27.499 | 62.483 | 17.605 | 1.00 | 48.21 | O |
| ATOM | 9070 | N | GLU | B | 196 | 28.455 | 64.127 | 16.440 | 1.00 | 48.78 | N |
| ATOM | 9072 | CA | GLU | B | 196 | 27.569 | 65.071 | 17.046 | 1.00 | 48.32 | C |
| ATOM | 9074 | CB | GLU | B | 196 | 28.199 | 66.421 | 17.091 | 1.00 | 47.83 | C |
| ATOM | 9077 | CG | GLU | B | 196 | 29.399 | 66.401 | 17.960 | 1.00 | 48.63 | C |
| ATOM | 9080 | CD | GLU | B | 196 | 30.197 | 67.634 | 17.756 | 1.00 | 47.85 | C |
| ATOM | 9081 | OE1 | GLU | B | 196 | 31.098 | 67.524 | 16.899 | 1.00 | 45.46 | O |
| ATOM | 9082 | OE2 | GLU | B | 196 | 29.909 | 68.667 | 18.442 | 1.00 | 49.47 | O |
| ATOM | 9083 | C | GLU | B | 196 | 26.332 | 65.193 | 16.240 | 1.00 | 48.04 | C |
| ATOM | 9084 | O | GLU | B | 196 | 26.225 | 66.119 | 15.433 | 1.00 | 51.46 | O |
| ATOM | 9085 | N | TRP | B | 197 | 25.402 | 64.269 | 16.438 | 1.00 | 43.41 | N |
| ATOM | 9087 | CA | TRP | B | 197 | 24.115 | 64.382 | 15.786 | 1.00 | 42.70 | C |
| ATOM | 9089 | CB | TRP | B | 197 | 24.013 | 63.512 | 14.546 | 1.00 | 42.18 | C |
| ATOM | 9092 | CG | TRP | B | 197 | 24.166 | 62.015 | 14.631 | 1.00 | 44.56 | C |
| ATOM | 9093 | CD1 | TRP | B | 197 | 25.015 | 61.311 | 15.385 | 1.00 | 48.17 | C |
| ATOM | 9095 | NE1 | TRP | B | 197 | 24.855 | 59.961 | 15.166 | 1.00 | 44.01 | N |
| ATOM | 9097 | CE2 | TRP | B | 197 | 23.874 | 59.782 | 14.237 | 1.00 | 41.09 | C |
| ATOM | 9098 | CD2 | TRP | B | 197 | 23.417 | 61.054 | 13.872 | 1.00 | 47.74 | C |
| ATOM | 9099 | CE3 | TRP | B | 197 | 22.389 | 61.153 | 12.917 | 1.00 | 49.53 | C |
| ATOM | 9101 | CZ3 | TRP | B | 197 | 21.865 | 59.993 | 12.371 | 1.00 | 47.08 | C |
| ATOM | 9103 | CH2 | TRP | B | 197 | 22.353 | 58.744 | 12.763 | 1.00 | 51.98 | C |
| ATOM | 9105 | CZ2 | TRP | B | 197 | 23.361 | 58.622 | 13.696 | 1.00 | 41.61 | C |
| ATOM | 9107 | C | TRP | B | 197 | 23.112 | 64.138 | 16.888 | 1.00 | 41.55 | C |
| ATOM | 9108 | O | TRP | B | 197 | 22.746 | 65.084 | 17.582 | 1.00 | 42.56 | O |
| ATOM | 9109 | N | TYR | B | 198 | 22.672 | 62.900 | 17.075 | 1.00 | 42.61 | N |
| ATOM | 9111 | CA | TYR | B | 198 | 22.025 | 62.545 | 18.329 | 1.00 | 41.33 | C |
| ATOM | 9113 | CB | TYR | B | 198 | 21.459 | 61.157 | 18.244 | 1.00 | 41.19 | C |
| ATOM | 9116 | CG | TYR | B | 198 | 20.254 | 61.024 | 17.390 | 1.00 | 39.49 | C |
| ATOM | 9117 | CD1 | TYR | B | 198 | 19.012 | 61.248 | 17.911 | 1.00 | 39.50 | C |
| ATOM | 9119 | CE1 | TYR | B | 198 | 17.901 | 61.130 | 17.152 | 1.00 | 38.38 | C |
| ATOM | 9121 | CZ | TYR | B | 198 | 18.010 | 60.768 | 15.848 | 1.00 | 37.08 | C |
| ATOM | 9122 | OH | TYR | B | 198 | 16.880 | 60.654 | 15.099 | 1.00 | 33.61 | O |
| ATOM | 9124 | CE2 | TYR | B | 198 | 19.229 | 60.528 | 15.293 | 1.00 | 42.60 | C |
| ATOM | 9126 | CD2 | TYR | B | 198 | 20.356 | 60.661 | 16.074 | 1.00 | 43.81 | C |
| ATOM | 9128 | C | TYR | B | 198 | 23.182 | 62.519 | 19.341 | 1.00 | 40.43 | C |
| ATOM | 9129 | O | TYR | B | 198 | 24.272 | 62.908 | 18.991 | 1.00 | 47.49 | O |
| ATOM | 9130 | N | TYR | B | 199 | 22.981 | 62.088 | 20.584 | 1.00 | 38.21 | N |
| ATOM | 9132 | CA | TYR | B | 199 | 24.128 | 61.846 | 21.436 | 1.00 | 35.93 | C |
| ATOM | 9134 | CB | TYR | B | 199 | 23.802 | 62.177 | 22.865 | 1.00 | 39.90 | C |
| ATOM | 9137 | CG | TYR | B | 199 | 23.751 | 63.668 | 23.174 | 1.00 | 43.36 | C |
| ATOM | 9138 | CD1 | TYR | B | 199 | 22.570 | 64.349 | 23.130 | 1.00 | 39.75 | C |
| ATOM | 9140 | CE1 | TYR | B | 199 | 22.500 | 65.679 | 23.408 | 1.00 | 42.44 | C |
| ATOM | 9142 | CZ | TYR | B | 199 | 23.616 | 66.367 | 23.741 | 1.00 | 46.22 | C |
| ATOM | 9143 | OH | TYR | B | 199 | 23.499 | 67.707 | 24.010 | 1.00 | 44.65 | O |
| ATOM | 9145 | CE2 | TYR | B | 199 | 24.832 | 65.726 | 23.794 | 1.00 | 48.47 | C |
| ATOM | 9147 | CD2 | TYR | B | 199 | 24.894 | 64.379 | 23.509 | 1.00 | 44.07 | C |
| ATOM | 9149 | C | TYR | B | 199 | 24.366 | 60.365 | 21.226 | 1.00 | 42.71 | C |
| ATOM | 9150 | O | TYR | B | 199 | 23.584 | 59.507 | 21.710 | 1.00 | 43.87 | O |
| ATOM | 9151 | N | GLU | B | 200 | 25.422 | 60.033 | 20.481 | 1.00 | 43.77 | N |
| ATOM | 9153 | CA | GLU | B | 200 | 25.573 | 58.653 | 19.995 | 1.00 | 42.26 | C |
| ATOM | 9155 | CB | GLU | B | 200 | 25.609 | 58.566 | 18.475 | 1.00 | 37.46 | C |
| ATOM | 9158 | CG | GLU | B | 200 | 25.766 | 57.159 | 17.955 | 1.00 | 37.24 | C |
| ATOM | 9161 | CD | GLU | B | 200 | 26.297 | 57.077 | 16.511 | 1.00 | 51.42 | C |
| ATOM | 9162 | OE1 | GLU | B | 200 | 26.309 | 58.100 | 15.756 | 1.00 | 59.01 | O |
| ATOM | 9163 | OE2 | GLU | B | 200 | 26.707 | 55.969 | 16.101 | 1.00 | 50.91 | O |
| ATOM | 9164 | C | GLU | B | 200 | 26.810 | 58.089 | 20.527 | 1.00 | 46.93 | C |
| ATOM | 9165 | O | GLU | B | 200 | 27.810 | 58.766 | 20.484 | 1.00 | 48.35 | O |
| ATOM | 9166 | N | VAL | B | 201 | 26.703 | 56.841 | 21.021 | 1.00 | 52.84 | N |
| ATOM | 9168 | CA | VAL | B | 201 | 27.773 | 56.090 | 21.669 | 1.00 | 48.82 | C |
| ATOM | 9170 | CB | VAL | B | 201 | 27.515 | 55.969 | 23.163 | 1.00 | 51.19 | C |
| ATOM | 9172 | CG1 | VAL | B | 201 | 27.484 | 57.332 | 23.864 | 1.00 | 51.45 | C |
| ATOM | 9176 | CG2 | VAL | B | 201 | 26.230 | 55.280 | 23.383 | 1.00 | 48.19 | C |
| ATOM | 9180 | C | VAL | B | 201 | 27.783 | 54.659 | 21.166 | 1.00 | 50.01 | C |
| ATOM | 9181 | O | VAL | B | 201 | 26.965 | 54.283 | 20.341 | 1.00 | 54.12 | O |
| ATOM | 9182 | N | ILE | B | 202 | 28.701 | 53.858 | 21.702 | 1.00 | 49.72 | N |
| ATOM | 9184 | CA | ILE | B | 202 | 28.945 | 52.497 | 21.257 | 1.00 | 45.16 | C |
| ATOM | 9186 | CB | ILE | B | 202 | 30.262 | 52.442 | 20.484 | 1.00 | 46.37 | C |
| ATOM | 9188 | CG1 | ILE | B | 202 | 30.075 | 52.858 | 19.028 | 1.00 | 47.26 | C |
| ATOM | 9191 | CD1 | ILE | B | 202 | 31.097 | 53.824 | 18.585 | 1.00 | 51.48 | C |
| ATOM | 9195 | CG2 | ILE | B | 202 | 30.727 | 51.098 | 20.431 | 1.00 | 39.79 | C |
| ATOM | 9199 | C | ILE | B | 202 | 29.052 | 51.546 | 22.401 | 1.00 | 42.02 | C |
| ATOM | 9200 | O | ILE | B | 202 | 29.706 | 51.788 | 23.359 | 1.00 | 44.16 | O |
| ATOM | 9201 | N | ILE | B | 203 | 28.429 | 50.423 | 22.252 | 1.00 | 42.56 | N |
| ATOM | 9203 | CA | ILE | B | 203 | 28.259 | 49.497 | 23.327 | 1.00 | 45.31 | C |
| ATOM | 9205 | CB | ILE | B | 203 | 26.823 | 49.150 | 23.567 | 1.00 | 43.41 | C |
| ATOM | 9207 | CG1 | ILE | B | 203 | 26.040 | 50.325 | 24.088 | 1.00 | 41.62 | C |
| ATOM | 9210 | CD1 | ILE | B | 203 | 24.600 | 49.972 | 24.158 | 1.00 | 42.16 | C |
| ATOM | 9214 | CG2 | ILE | B | 203 | 26.780 | 48.073 | 24.599 | 1.00 | 44.28 | C |
| ATOM | 9218 | C | ILE | B | 203 | 28.888 | 48.250 | 22.866 | 1.00 | 49.14 | C |
| ATOM | 9219 | O | ILE | B | 203 | 28.537 | 47.712 | 21.806 | 1.00 | 47.82 | O |
| ATOM | 9220 | N | VAL | B | 204 | 29.782 | 47.762 | 23.696 | 1.00 | 49.32 | N |
| ATOM | 9222 | CA | VAL | B | 204 | 30.610 | 46.689 | 23.294 | 1.00 | 48.88 | C |
| ATOM | 9224 | CB | VAL | B | 204 | 31.996 | 47.004 | 23.720 | 1.00 | 45.35 | C |
| ATOM | 9226 | CG1 | VAL | B | 204 | 32.441 | 48.273 | 23.012 | 1.00 | 49.69 | C |
| ATOM | 9230 | CG2 | VAL | B | 204 | 32.055 | 47.203 | 25.163 | 1.00 | 44.93 | C |
| ATOM | 9234 | C | VAL | B | 204 | 30.179 | 45.382 | 23.866 | 1.00 | 51.35 | C |
| ATOM | 9235 | O | VAL | B | 204 | 30.565 | 44.349 | 23.337 | 1.00 | 57.30 | O |
| ATOM | 9236 | N | ARG | B | 205 | 29.392 | 45.397 | 24.941 | 1.00 | 50.88 | N |
| ATOM | 9238 | CA | ARG | B | 205 | 29.044 | 44.122 | 25.568 | 1.00 | 52.74 | C |
| ATOM | 9240 | CB | ARG | B | 205 | 30.299 | 43.498 | 26.174 | 1.00 | 52.53 | C |
| ATOM | 9243 | CG | ARG | B | 205 | 30.064 | 42.536 | 27.318 | 1.00 | 54.44 | C |
| ATOM | 9246 | CD | ARG | B | 205 | 31.354 | 41.863 | 27.754 | 1.00 | 56.13 | C |
| ATOM | 9249 | NE | ARG | B | 205 | 31.296 | 41.266 | 29.087 | 1.00 | 59.87 | N |
| ATOM | 9251 | CZ | ARG | B | 205 | 31.742 | 40.048 | 29.378 | 1.00 | 59.78 | C |
| ATOM | 9252 | NH1 | ARG | B | 205 | 32.275 | 39.296 | 28.429 | 1.00 | 58.07 | N |
| ATOM | 9255 | NH2 | ARG | B | 205 | 31.657 | 39.585 | 30.618 | 1.00 | 61.85 | N |
| ATOM | 9258 | C | ARG | B | 205 | 27.953 | 44.177 | 26.605 | 1.00 | 50.44 | C |
| ATOM | 9259 | O | ARG | B | 205 | 28.004 | 44.985 | 27.512 | 1.00 | 60.70 | O |
| ATOM | 9260 | N | VAL | B | 206 | 26.972 | 43.298 | 26.455 | 1.00 | 48.83 | N |
| ATOM | 9262 | CA | VAL | B | 206 | 25.846 | 43.189 | 27.373 | 1.00 | 48.98 | C |
| ATOM | 9264 | CB | VAL | B | 206 | 24.544 | 43.113 | 26.609 | 1.00 | 47.56 | C |
| ATOM | 9266 | CG1 | VAL | B | 206 | 23.429 | 42.948 | 27.571 | 1.00 | 51.41 | C |
| ATOM | 9270 | CG2 | VAL | B | 206 | 24.310 | 44.358 | 25.719 | 1.00 | 47.81 | C |
| ATOM | 9274 | C | VAL | B | 206 | 25.931 | 41.888 | 28.204 | 1.00 | 49.62 | C |
| ATOM | 9275 | O | VAL | B | 206 | 26.324 | 40.839 | 27.707 | 1.00 | 51.17 | O |
| ATOM | 9276 | N | GLU | B | 207 | 25.549 | 41.972 | 29.466 | 1.00 | 48.53 | N |
| ATOM | 9278 | CA | GLU | B | 207 | 25.511 | 40.846 | 30.346 | 1.00 | 50.25 | C |
| ATOM | 9280 | CB | GLU | B | 207 | 26.588 | 40.933 | 31.415 | 1.00 | 50.16 | C |
| ATOM | 9283 | CG | GLU | B | 207 | 27.966 | 40.533 | 30.976 | 1.00 | 49.74 | C |
| ATOM | 9286 | CD | GLU | B | 207 | 28.971 | 40.942 | 32.003 | 1.00 | 49.93 | C |
| ATOM | 9287 | OE1 | GLU | B | 207 | 28.551 | 41.333 | 33.115 | 1.00 | 53.30 | O |
| ATOM | 9288 | OE2 | GLU | B | 207 | 30.168 | 40.887 | 31.697 | 1.00 | 51.43 | O |
| ATOM | 9289 | C | GLU | B | 207 | 24.194 | 40.846 | 31.063 | 1.00 | 53.82 | C |
| ATOM | 9290 | O | GLU | B | 207 | 23.625 | 41.879 | 31.314 | 1.00 | 53.60 | O |
| ATOM | 9291 | N | ILE | B | 208 | 23.715 | 39.664 | 31.408 | 1.00 | 56.66 | N |
| ATOM | 9293 | CA | ILE | B | 208 | 22.535 | 39.565 | 32.224 | 1.00 | 54.83 | C |
| ATOM | 9295 | CB | ILE | B | 208 | 21.422 | 38.952 | 31.434 | 1.00 | 55.18 | C |
| ATOM | 9297 | CG1 | ILE | B | 208 | 21.118 | 39.839 | 30.212 | 1.00 | 56.27 | C |
| ATOM | 9300 | CD1 | ILE | B | 208 | 20.393 | 41.124 | 30.503 | 1.00 | 49.77 | C |
| ATOM | 9304 | CG2 | ILE | B | 208 | 20.205 | 38.785 | 32.309 | 1.00 | 57.22 | C |
| ATOM | 9308 | C | ILE | B | 208 | 22.955 | 38.724 | 33.429 | 1.00 | 57.88 | C |
| ATOM | 9309 | O | ILE | B | 208 | 23.347 | 37.560 | 33.302 | 1.00 | 57.17 | O |
| ATOM | 9310 | N | ASN | B | 209 | 22.883 | 39.337 | 34.603 | 1.00 | 54.93 | N |
| ATOM | 9312 | CA | ASN | B | 209 | 23.319 | 38.694 | 35.835 | 1.00 | 56.25 | C |
| ATOM | 9314 | CB | ASN | B | 209 | 22.270 | 37.722 | 36.372 | 1.00 | 54.61 | C |
| ATOM | 9317 | CG | ASN | B | 209 | 21.639 | 38.210 | 37.663 | 1.00 | 55.46 | C |
| ATOM | 9318 | OD1 | ASN | B | 209 | 21.992 | 39.277 | 38.187 | 1.00 | 50.67 | O |
| ATOM | 9319 | ND2 | ASN | B | 209 | 20.695 | 37.428 | 38.189 | 1.00 | 53.95 | N |
| ATOM | 9322 | C | ASN | B | 209 | 24.668 | 38.015 | 35.668 | 1.00 | 52.94 | C |
| ATOM | 9323 | O | ASN | B | 209 | 24.881 | 36.887 | 36.076 | 1.00 | 47.18 | O |
| ATOM | 9324 | N | GLY | B | 210 | 25.581 | 38.741 | 35.055 | 1.00 | 51.75 | N |
| ATOM | 9326 | CA | GLY | B | 210 | 26.943 | 38.274 | 34.887 | 1.00 | 47.80 | C |
| ATOM | 9329 | C | GLY | B | 210 | 27.140 | 37.488 | 33.626 | 1.00 | 46.54 | C |
| ATOM | 9330 | O | GLY | B | 210 | 28.279 | 37.328 | 33.187 | 1.00 | 40.63 | O |
| ATOM | 9331 | N | GLN | B | 211 | 26.035 | 37.004 | 33.051 | 1.00 | 50.65 | N |
| ATOM | 9333 | CA | GLN | B | 211 | 26.080 | 36.138 | 31.863 | 1.00 | 53.20 | C |
| ATOM | 9335 | CB | GLN | B | 211 | 24.983 | 35.077 | 31.888 | 1.00 | 52.67 | C |
| ATOM | 9338 | CG | GLN | B | 211 | 24.850 | 34.430 | 30.515 | 1.00 | 63.08 | C |
| ATOM | 9341 | CD | GLN | B | 211 | 24.441 | 32.964 | 30.550 | 1.00 | 65.01 | C |
| ATOM | 9342 | OE1 | GLN | B | 211 | 23.518 | 32.591 | 31.263 | 1.00 | 67.35 | O |
| ATOM | 9343 | NE2 | GLN | B | 211 | 25.123 | 32.142 | 29.767 | 1.00 | 70.29 | N |
| ATOM | 9346 | C | GLN | B | 211 | 25.986 | 36.886 | 30.522 | 1.00 | 54.37 | C |
| ATOM | 9347 | O | GLN | B | 211 | 24.946 | 37.452 | 30.180 | 1.00 | 54.93 | O |
| ATOM | 9348 | N | ASP | B | 212 | 27.098 | 36.852 | 29.788 | 1.00 | 52.76 | N |
| ATOM | 9350 | CA | ASP | B | 212 | 27.269 | 37.451 | 28.466 | 1.00 | 56.79 | C |
| ATOM | 9352 | CB | ASP | B | 212 | 28.662 | 37.038 | 27.957 | 1.00 | 54.29 | C |
| ATOM | 9355 | CG | ASP | B | 212 | 29.016 | 37.650 | 26.623 | 1.00 | 63.19 | C |
| ATOM | 9356 | OD1 | ASP | B | 212 | 28.177 | 38.399 | 26.067 | 1.00 | 70.42 | O |
| ATOM | 9357 | OD2 | ASP | B | 212 | 30.120 | 37.448 | 26.044 | 1.00 | 67.39 | O |
| ATOM | 9358 | C | ASP | B | 212 | 26.189 | 37.063 | 27.435 | 1.00 | 61.19 | C |
| ATOM | 9359 | O | ASP | B | 212 | 25.844 | 35.877 | 27.280 | 1.00 | 54.41 | O |
| ATOM | 9360 | N | LEU | B | 213 | 25.680 | 38.092 | 26.738 | 1.00 | 62.11 | N |
| ATOM | 9362 | CA | LEU | B | 213 | 24.673 | 37.937 | 25.688 | 1.00 | 61.19 | C |
| ATOM | 9364 | CB | LEU | B | 213 | 24.155 | 39.306 | 25.248 | 1.00 | 62.39 | C |
| ATOM | 9367 | CG | LEU | B | 213 | 22.628 | 39.359 | 25.155 | 1.00 | 58.53 | C |
| ATOM | 9369 | CD1 | LEU | B | 213 | 22.077 | 38.183 | 25.895 | 1.00 | 57.55 | C |
| ATOM | 9373 | CD2 | LEU | B | 213 | 22.041 | 40.662 | 25.697 | 1.00 | 55.91 | C |
| ATOM | 9377 | C | LEU | B | 213 | 25.319 | 37.210 | 24.534 | 1.00 | 63.18 | C |
| ATOM | 9378 | O | LYS | B | 213 | 24.671 | 36.470 | 23.794 | 1.00 | 57.02 | O |
| ATOM | 9379 | N | LYS | B | 214 | 26.619 | 37.447 | 24.387 | 1.00 | 66.53 | N |
| ATOM | 9381 | CA | LYS | B | 214 | 27.466 | 36.603 | 23.539 | 1.00 | 70.91 | C |
| ATOM | 9383 | CB | LYS | B | 214 | 27.309 | 35.139 | 23.941 | 1.00 | 69.78 | C |
| ATOM | 9386 | CG | LYS | B | 214 | 28.397 | 34.242 | 23.410 | 1.00 | 71.78 | C |
| ATOM | 9389 | CD | LYS | B | 214 | 28.266 | 32.838 | 23.992 | 1.00 | 72.93 | C |
| ATOM | 9392 | CE | LYS | B | 214 | 28.927 | 31.796 | 23.116 | 1.00 | 70.34 | C |
| ATOM | 9395 | NZ | LYS | B | 214 | 27.903 | 31.146 | 22.264 | 1.00 | 64.34 | N |
| ATOM | 9399 | C | LYS | B | 214 | 27.143 | 36.780 | 22.072 | 1.00 | 73.22 | C |
| ATOM | 9400 | O | LYS | B | 214 | 27.246 | 35.850 | 21.251 | 1.00 | 66.62 | O |
| ATOM | 9401 | N | MET | B | 215 | 26.757 | 38.011 | 21.774 | 1.00 | 75.18 | N |
| ATOM | 9403 | CA | MET | B | 215 | 26.432 | 38.407 | 20.440 | 1.00 | 73.26 | C |
| ATOM | 9405 | CB | MET | B | 215 | 25.290 | 39.408 | 20.449 | 1.00 | 72.49 | C |
| ATOM | 9408 | CG | MET | B | 215 | 23.998 | 38.728 | 20.268 | 1.00 | 72.86 | C |
| ATOM | 9411 | SD | MET | B | 215 | 22.683 | 39.755 | 20.640 | 1.00 | 75.68 | S |
| ATOM | 9412 | CE | MET | B | 215 | 21.602 | 39.194 | 19.427 | 1.00 | 75.77 | C |
| ATOM | 9416 | C | MET | B | 215 | 27.642 | 39.092 | 19.959 | 1.00 | 73.98 | C |
| ATOM | 9417 | O | MET | B | 215 | 28.518 | 39.477 | 20.760 | 1.00 | 71.14 | O |
| ATOM | 9418 | N | ASP | B | 216 | 27.691 | 39.237 | 18.644 | 1.00 | 72.78 | N |
| ATOM | 9420 | CA | ASP | B | 216 | 28.719 | 40.037 | 18.037 | 1.00 | 71.14 | C |
| ATOM | 9422 | CB | ASP | B | 216 | 28.767 | 39.744 | 16.540 | 1.00 | 71.31 | C |
| ATOM | 9425 | CG | ASP | B | 216 | 29.577 | 40.749 | 15.771 | 1.00 | 74.43 | C |
| ATOM | 9426 | OD1 | ASP | B | 216 | 30.271 | 41.594 | 16.394 | 1.00 | 80.47 | O |
| ATOM | 9427 | OD2 | ASP | B | 216 | 29.584 | 40.763 | 14.524 | 1.00 | 72.09 | O |
| ATOM | 9428 | C | ASP | B | 216 | 28.186 | 41.415 | 18.401 | 1.00 | 68.29 | C |
| ATOM | 9429 | O | ASP | B | 216 | 26.983 | 41.655 | 18.318 | 1.00 | 62.61 | O |
| ATOM | 9430 | N | CYS | B | 217 | 29.061 | 42.308 | 18.831 | 1.00 | 68.51 | N |
| ATOM | 9432 | CA | CYS | B | 217 | 28.625 | 43.609 | 19.316 | 1.00 | 68.58 | C |
| ATOM | 9434 | CB | CYS | B | 217 | 29.795 | 44.285 | 19.994 | 1.00 | 68.63 | C |
| ATOM | 9437 | SG | CYS | B | 217 | 31.286 | 44.299 | 18.997 | 1.00 | 74.46 | S |
| ATOM | 9438 | C | CYS | B | 217 | 28.007 | 44.565 | 18.282 | 1.00 | 70.29 | C |
| ATOM | 9439 | O | CYS | B | 217 | 27.287 | 45.509 | 18.640 | 1.00 | 68.93 | O |
| ATOM | 9440 | N | LYS | B | 218 | 28.244 | 44.334 | 17.002 | 1.00 | 71.63 | N |
| ATOM | 9442 | CA | LYS | B | 218 | 27.750 | 45.284 | 16.014 | 1.00 | 70.37 | C |
| ATOM | 9444 | CB | LYS | B | 218 | 28.259 | 44.933 | 14.611 | 1.00 | 71.99 | C |
| ATOM | 9447 | CG | LYS | B | 218 | 29.041 | 46.076 | 13.943 | 1.00 | 76.53 | C |
| ATOM | 9450 | CD | LYS | B | 218 | 29.452 | 45.730 | 12.496 | 1.00 | 80.60 | C |
| ATOM | 9453 | CE | LYS | B | 218 | 30.493 | 46.715 | 11.896 | 1.00 | 83.08 | C |
| ATOM | 9456 | NZ | LYS | B | 218 | 31.690 | 46.955 | 12.763 | 1.00 | 84.11 | N |
| ATOM | 9460 | C | LYS | B | 218 | 26.229 | 45.322 | 16.063 | 1.00 | 66.79 | C |
| ATOM | 9461 | O | LYS | B | 218 | 25.578 | 46.202 | 15.509 | 1.00 | 63.62 | O |
| ATOM | 9462 | N | GLU | B | 219 | 25.659 | 44.352 | 16.743 | 1.00 | 64.08 | N |
| ATOM | 9464 | CA | GLU | B | 219 | 24.222 | 44.244 | 16.800 | 1.00 | 66.34 | C |
| ATOM | 9466 | CB | GLU | B | 219 | 23.839 | 42.798 | 17.059 | 1.00 | 69.58 | C |
| ATOM | 9469 | CG | GLU | B | 219 | 24.551 | 41.798 | 16.166 | 1.00 | 71.88 | C |
| ATOM | 9472 | CD | GLU | B | 219 | 23.800 | 41.529 | 14.875 | 1.00 | 78.40 | C |
| ATOM | 9473 | OE1 | GLU | B | 219 | 22.637 | 42.002 | 14.721 | 1.00 | 72.86 | O |
| ATOM | 9474 | OE2 | GLU | B | 219 | 24.389 | 40.835 | 14.009 | 1.00 | 85.55 | O |
| ATOM | 9475 | C | GLU | B | 219 | 23.691 | 45.065 | 17.931 | 1.00 | 65.05 | C |
| ATOM | 9476 | O | GLU | B | 219 | 22.528 | 45.438 | 17.961 | 1.00 | 63.97 | O |
| ATOM | 9477 | N | TYR | B | 220 | 24.550 | 45.342 | 18.888 | 1.00 | 65.15 | N |
| ATOM | 9479 | CA | TYR | B | 220 | 24.080 | 45.993 | 20.070 | 1.00 | 62.09 | C |
| ATOM | 9481 | CB | TYR | B | 220 | 25.134 | 45.900 | 21.179 | 1.00 | 62.12 | C |
| ATOM | 9484 | CG | TYR | B | 220 | 25.409 | 44.536 | 21.845 | 1.00 | 56.86 | C |
| ATOM | 9485 | CD1 | TYR | B | 220 | 26.716 | 44.158 | 22.123 | 1.00 | 60.11 | C |
| ATOM | 9487 | CE1 | TYR | B | 220 | 27.019 | 42.952 | 22.738 | 1.00 | 59.02 | C |
| ATOM | 9489 | CZ | TYR | B | 220 | 26.013 | 42.097 | 23.090 | 1.00 | 60.03 | C |
| ATOM | 9490 | OH | TYR | B | 220 | 26.387 | 40.915 | 23.694 | 1.00 | 65.94 | O |
| ATOM | 9492 | CE2 | TYR | B | 220 | 24.689 | 42.438 | 22.840 | 1.00 | 57.10 | C |
| ATOM | 9494 | CD2 | TYR | B | 220 | 24.396 | 43.658 | 22.216 | 1.00 | 55.80 | C |
| ATOM | 9496 | C | TYR | B | 220 | 23.817 | 47.430 | 19.628 | 1.00 | 61.17 | C |
| ATOM | 9497 | O | TYR | B | 220 | 22.944 | 48.103 | 20.158 | 1.00 | 64.67 | O |
| ATOM | 9498 | N | ASN | B | 221 | 24.569 | 47.880 | 18.628 | 1.00 | 59.63 | N |
| ATOM | 9500 | CA | ASN | B | 221 | 24.486 | 49.244 | 18.114 | 1.00 | 56.38 | C |
| ATOM | 9502 | CB | ASN | B | 221 | 25.865 | 49.780 | 18.037 | 1.00 | 53.93 | C |
| ATOM | 9505 | CG | ASN | B | 221 | 26.510 | 49.843 | 19.322 | 1.00 | 51.48 | C |
| ATOM | 9506 | OD1 | ASN | B | 221 | 26.001 | 50.434 | 20.257 | 1.00 | 51.50 | O |
| ATOM | 9507 | ND2 | ASN | B | 221 | 27.686 | 49.245 | 19.405 | 1.00 | 56.63 | N |
| ATOM | 9510 | C | ASN | B | 221 | 23.971 | 49.285 | 16.692 | 1.00 | 58.77 | C |
| ATOM | 9511 | O | ASN | B | 221 | 24.232 | 50.236 | 15.890 | 1.00 | 59.98 | O |
| ATOM | 9512 | N | TYR | B | 222 | 23.235 | 48.249 | 16.362 | 1.00 | 59.18 | N |
| ATOM | 9514 | CA | TYR | B | 222 | 22.870 | 48.096 | 14.998 | 1.00 | 60.10 | C |
| ATOM | 9516 | CB | TYR | B | 222 | 22.291 | 46.743 | 14.649 | 1.00 | 64.25 | C |
| ATOM | 9519 | CG | TYR | B | 222 | 21.579 | 46.790 | 13.336 | 1.00 | 65.11 | C |
| ATOM | 9520 | CD1 | TYR | B | 222 | 22.008 | 47.632 | 12.328 | 1.00 | 67.45 | C |
| ATOM | 9522 | CE1 | TYR | B | 222 | 21.356 | 47.679 | 11.135 | 1.00 | 68.44 | C |
| ATOM | 9524 | CZ | TYR | B | 222 | 20.261 | 46.875 | 10.941 | 1.00 | 68.04 | C |
| ATOM | 9525 | OH | TYR | B | 222 | 19.605 | 46.913 | 9.760 | 1.00 | 72.86 | O |
| ATOM | 9527 | CE2 | TYR | B | 222 | 19.814 | 46.037 | 11.916 | 1.00 | 65.98 | C |
| ATOM | 9529 | CD2 | TYR | B | 222 | 20.470 | 45.999 | 13.105 | 1.00 | 66.41 | C |
| ATOM | 9531 | C | TYR | B | 222 | 21.922 | 49.157 | 14.671 | 1.00 | 57.69 | C |
| ATOM | 9532 | O | TYR | B | 222 | 20.737 | 49.117 | 14.993 | 1.00 | 57.52 | O |
| ATOM | 9533 | N | ASP | B | 223 | 22.571 | 50.101 | 14.023 | 1.00 | 58.59 | N |
| ATOM | 9535 | CA | ASP | B | 223 | 22.058 | 51.250 | 13.316 | 1.00 | 57.20 | C |
| ATOM | 9537 | CB | ASP | B | 223 | 20.511 | 51.264 | 13.178 | 1.00 | 62.18 | C |
| ATOM | 9540 | CG | ASP | B | 223 | 19.929 | 52.514 | 12.350 | 1.00 | 65.70 | C |
| ATOM | 9541 | OD1 | ASP | B | 223 | 19.045 | 53.192 | 12.919 | 1.00 | 72.63 | O |
| ATOM | 9542 | OD2 | ASP | B | 223 | 20.243 | 52.884 | 11.172 | 1.00 | 60.60 | O |
| ATOM | 9543 | C | ASP | B | 223 | 22.694 | 52.313 | 14.142 | 1.00 | 52.54 | C |
| ATOM | 9544 | O | ASP | B | 223 | 23.514 | 53.049 | 13.659 | 1.00 | 53.53 | O |
| ATOM | 9545 | N | LYS | B | 224 | 22.364 | 52.357 | 15.416 | 1.00 | 55.48 | N |
| ATOM | 9547 | CA | LYS | B | 224 | 22.905 | 53.373 | 16.312 | 1.00 | 53.90 | C |
| ATOM | 9549 | CB | LYS | B | 224 | 22.306 | 54.743 | 16.002 | 1.00 | 55.04 | C |
| ATOM | 9552 | CG | LYS | B | 224 | 20.794 | 54.865 | 16.179 | 1.00 | 52.13 | C |
| ATOM | 9555 | CD | LYS | B | 224 | 20.386 | 56.306 | 15.817 | 1.00 | 47.46 | C |
| ATOM | 9558 | CE | LYS | B | 224 | 18.962 | 56.435 | 15.312 | 1.00 | 47.23 | C |
| ATOM | 9561 | NZ | LYS | B | 224 | 18.922 | 56.181 | 13.825 | 1.00 | 47.81 | N |
| ATOM | 9565 | C | LYS | B | 224 | 22.640 | 53.044 | 17.752 | 1.00 | 55.74 | C |
| ATOM | 9566 | O | LYS | B | 224 | 22.165 | 51.982 | 18.073 | 1.00 | 56.88 | O |
| ATOM | 9567 | N | SER | B | 225 | 22.974 | 53.966 | 18.632 | 1.00 | 59.75 | N |
| ATOM | 9569 | CA | SER | B | 225 | 22.735 | 53.775 | 20.046 | 1.00 | 58.65 | C |
| ATOM | 9571 | CB | SER | B | 225 | 23.820 | 52.902 | 20.666 | 1.00 | 58.31 | C |
| ATOM | 9574 | OG | SER | B | 225 | 23.410 | 51.542 | 20.653 | 1.00 | 64.32 | O |
| ATOM | 9576 | C | SER | B | 225 | 22.795 | 55.133 | 20.625 | 1.00 | 51.40 | C |
| ATOM | 9577 | O | SER | B | 225 | 23.838 | 55.743 | 20.585 | 1.00 | 52.21 | O |
| ATOM | 9578 | N | ILE | B | 226 | 21.681 | 55.609 | 21.159 | 1.00 | 48.97 | N |
| ATOM | 9580 | CA | ILE | B | 226 | 21.652 | 56.966 | 21.677 | 1.00 | 47.18 | C |
| ATOM | 9582 | CB | ILE | B | 226 | 20.782 | 57.870 | 20.775 | 1.00 | 42.41 | C |
| ATOM | 9584 | CG1 | ILE | B | 226 | 19.421 | 57.276 | 20.563 | 1.00 | 36.20 | C |
| ATOM | 9587 | CD1 | ILE | B | 226 | 18.355 | 58.275 | 20.220 | 1.00 | 36.28 | C |
| ATOM | 9591 | CG2 | ILE | B | 226 | 21.425 | 58.015 | 19.433 | 1.00 | 45.19 | C |
| ATOM | 9595 | C | ILE | B | 226 | 21.191 | 57.023 | 23.126 | 1.00 | 45.97 | C |
| ATOM | 9596 | O | ILE | B | 226 | 20.610 | 56.051 | 23.598 | 1.00 | 43.09 | O |
| ATOM | 9597 | N | VAL | B | 227 | 21.491 | 58.152 | 23.797 | 1.00 | 43.58 | N |
| ATOM | 9599 | CA | VAL | B | 227 | 21.061 | 58.457 | 25.177 | 1.00 | 44.91 | C |
| ATOM | 9601 | CB | VAL | B | 227 | 22.211 | 59.070 | 26.001 | 1.00 | 40.77 | C |
| ATOM | 9603 | CG1 | VAL | B | 227 | 21.726 | 59.493 | 27.313 | 1.00 | 40.80 | C |
| ATOM | 9607 | CG2 | VAL | B | 227 | 23.360 | 58.096 | 26.187 | 1.00 | 38.82 | C |
| ATOM | 9611 | C | VAL | B | 227 | 19.926 | 59.511 | 25.104 | 1.00 | 48.95 | C |
| ATOM | 9612 | O | VAL | B | 227 | 20.150 | 60.617 | 24.596 | 1.00 | 50.25 | O |
| ATOM | 9613 | N | ASP | B | 228 | 18.730 | 59.205 | 25.624 | 1.00 | 44.84 | N |
| ATOM | 9615 | CA | ASP | B | 228 | 17.565 | 60.043 | 25.336 | 1.00 | 40.47 | C |
| ATOM | 9617 | CB | ASP | B | 228 | 16.936 | 59.514 | 24.036 | 1.00 | 38.92 | C |
| ATOM | 9620 | CG | ASP | B | 228 | 15.690 | 60.286 | 23.615 | 1.00 | 45.59 | C |
| ATOM | 9621 | OD1 | ASP | B | 228 | 15.409 | 61.367 | 24.161 | 1.00 | 41.67 | O |
| ATOM | 9622 | OD2 | ASP | B | 228 | 14.903 | 59.871 | 22.744 | 1.00 | 55.53 | O |
| ATOM | 9623 | C | ASP | B | 228 | 16.512 | 60.133 | 26.470 | 1.00 | 43.36 | C |
| ATOM | 9624 | O | ASP | B | 228 | 15.725 | 59.217 | 26.694 | 1.00 | 38.56 | O |
| ATOM | 9625 | N | SER | B | 229 | 16.488 | 61.267 | 27.164 | 1.00 | 47.36 | N |
| ATOM | 9627 | CA | SER | B | 229 | 15.612 | 61.464 | 28.310 | 1.00 | 47.52 | C |
| ATOM | 9629 | CB | SER | B | 229 | 16.147 | 62.621 | 29.118 | 1.00 | 47.91 | C |
| ATOM | 9632 | OG | SER | B | 229 | 16.818 | 63.473 | 28.231 | 1.00 | 51.18 | O |
| ATOM | 9634 | C | SER | B | 229 | 14.158 | 61.708 | 27.934 | 1.00 | 49.38 | C |
| ATOM | 9635 | O | SER | B | 229 | 13.330 | 61.994 | 28.798 | 1.00 | 54.96 | O |
| ATOM | 9636 | N | GLY | B | 230 | 13.854 | 61.632 | 26.644 | 1.00 | 48.92 | N |
| ATOM | 9638 | CA | GLY | B | 230 | 12.481 | 61.753 | 26.176 | 1.00 | 45.57 | C |
| ATOM | 9641 | C | GLY | B | 230 | 11.935 | 60.429 | 25.637 | 1.00 | 48.29 | C |
| ATOM | 9642 | O | GLY | B | 230 | 10.833 | 60.372 | 25.085 | 1.00 | 40.94 | O |
| ATOM | 9643 | N | THR | B | 231 | 12.735 | 59.372 | 25.785 | 1.00 | 45.26 | N |
| ATOM | 9645 | CA | THR | B | 231 | 12.303 | 58.035 | 25.516 | 1.00 | 43.47 | C |
| ATOM | 9647 | CB | THR | B | 231 | 13.349 | 57.260 | 24.743 | 1.00 | 44.17 | C |
| ATOM | 9649 | OG1 | THR | B | 231 | 13.221 | 57.526 | 23.350 | 1.00 | 52.04 | O |
| ATOM | 9651 | CG2 | THR | B | 231 | 13.075 | 55.769 | 24.829 | 1.00 | 43.12 | C |
| ATOM | 9655 | C | THR | B | 231 | 12.171 | 57.381 | 26.865 | 1.00 | 43.15 | C |
| ATOM | 9656 | O | THR | B | 231 | 13.088 | 57.379 | 27.628 | 1.00 | 49.59 | O |
| ATOM | 9657 | N | THR | B | 232 | 11.031 | 56.804 | 27.149 | 1.00 | 45.08 | N |
| ATOM | 9659 | CA | THR | B | 232 | 10.801 | 56.103 | 28.393 | 1.00 | 46.07 | C |
| ATOM | 9661 | CB | THR | B | 232 | 9.344 | 55.581 | 28.375 | 1.00 | 43.69 | C |
| ATOM | 9663 | OG1 | THR | B | 232 | 8.475 | 56.653 | 27.964 | 1.00 | 49.53 | O |
| ATOM | 9665 | CG2 | THR | B | 232 | 8.867 | 55.276 | 29.748 | 1.00 | 43.76 | C |
| ATOM | 9669 | C | THR | B | 232 | 11.759 | 54.905 | 28.630 | 1.00 | 49.16 | C |
| ATOM | 9670 | O | THR | B | 232 | 12.563 | 54.922 | 29.567 | 1.00 | 51.59 | O |
| ATOM | 9671 | N | ASN | B | 233 | 11.660 | 53.899 | 27.757 | 1.00 | 51.81 | N |
| ATOM | 9673 | CA | ASN | B | 233 | 12.327 | 52.604 | 27.879 | 1.00 | 50.18 | C |
| ATOM | 9675 | CB | ASN | B | 233 | 11.655 | 51.566 | 26.967 | 1.00 | 52.42 | C |
| ATOM | 9678 | CG | ASN | B | 233 | 10.276 | 51.148 | 27.383 | 1.00 | 52.34 | C |
| ATOM | 9679 | OD1 | ASN | B | 233 | 9.680 | 51.677 | 28.321 | 1.00 | 50.82 | O |
| ATOM | 9680 | ND2 | ASN | B | 233 | 9.750 | 50.153 | 26.650 | 1.00 | 52.61 | N |
| ATOM | 9683 | C | ASN | B | 233 | 13.729 | 52.350 | 27.416 | 1.00 | 47.14 | C |
| ATOM | 9684 | O | ASN | B | 233 | 14.259 | 52.963 | 26.555 | 1.00 | 46.63 | O |
| ATOM | 9685 | N | LEU | B | 234 | 14.306 | 51.355 | 28.019 | 1.00 | 47.70 | N |
| ATOM | 9687 | CA | LEU | B | 234 | 15.430 | 50.768 | 27.402 | 1.00 | 51.03 | C |
| ATOM | 9689 | CB | LEU | B | 234 | 15.903 | 49.591 | 28.256 | 1.00 | 47.48 | C |
| ATOM | 9692 | CG | LEU | B | 234 | 17.388 | 49.421 | 28.108 | 1.00 | 44.91 | C |
| ATOM | 9694 | CD1 | LEU | B | 234 | 17.926 | 48.199 | 28.788 | 1.00 | 47.69 | C |
| ATOM | 9698 | CD2 | LEU | B | 234 | 17.643 | 49.365 | 26.676 | 1.00 | 49.15 | C |
| ATOM | 9702 | C | LEU | B | 234 | 14.716 | 50.251 | 26.142 | 1.00 | 53.66 | C |
| ATOM | 9703 | O | LEU | B | 234 | 13.639 | 49.694 | 26.270 | 1.00 | 57.67 | O |
| ATOM | 9704 | N | ARG | B | 235 | 15.262 | 50.431 | 24.945 | 1.00 | 54.47 | N |
| ATOM | 9706 | CA | ARG | B | 235 | 14.628 | 49.892 | 23.749 | 1.00 | 50.55 | C |
| ATOM | 9708 | CB | ARG | B | 235 | 14.146 | 50.948 | 22.782 | 1.00 | 54.70 | C |
| ATOM | 9711 | CG | ARG | B | 235 | 13.090 | 51.958 | 23.232 | 1.00 | 60.77 | C |
| ATOM | 9714 | CD | ARG | B | 235 | 11.610 | 51.498 | 23.022 | 1.00 | 63.38 | C |
| ATOM | 9717 | NE | ARG | B | 235 | 10.905 | 52.094 | 21.882 | 1.00 | 65.81 | N |
| ATOM | 9719 | CZ | ARG | B | 235 | 10.487 | 51.422 | 20.806 | 1.00 | 68.02 | C |
| ATOM | 9720 | NH1 | ARG | B | 235 | 9.854 | 52.062 | 19.844 | 1.00 | 70.13 | N |
| ATOM | 9723 | NH2 | ARG | B | 235 | 10.703 | 50.121 | 20.678 | 1.00 | 68.31 | N |
| ATOM | 9726 | C | ARG | B | 235 | 15.799 | 49.326 | 23.069 | 1.00 | 52.73 | C |
| ATOM | 9727 | O | ARG | B | 235 | 16.794 | 50.033 | 22.960 | 1.00 | 51.89 | O |
| ATOM | 9728 | N | LEU | B | 236 | 15.697 | 48.083 | 22.591 | 1.00 | 53.67 | N |
| ATOM | 9730 | CA | LEU | B | 236 | 16.816 | 47.414 | 21.951 | 1.00 | 51.18 | C |
| ATOM | 9732 | CB | LEU | B | 236 | 17.336 | 46.273 | 22.857 | 1.00 | 54.12 | C |
| ATOM | 9735 | CG | LEU | B | 236 | 17.606 | 46.565 | 24.346 | 1.00 | 54.84 | C |
| ATOM | 9737 | CD1 | LEU | B | 236 | 17.445 | 45.294 | 25.170 | 1.00 | 59.47 | C |
| ATOM | 9741 | CD2 | LEU | B | 236 | 18.988 | 47.171 | 24.608 | 1.00 | 57.80 | C |
| ATOM | 9745 | C | LEU | B | 236 | 16.472 | 46.882 | 20.555 | 1.00 | 49.66 | C |
| ATOM | 9746 | O | LEU | B | 236 | 15.320 | 46.574 | 20.244 | 1.00 | 51.23 | O |
| ATOM | 9747 | N | PRO | B | 237 | 17.516 | 46.802 | 19.740 | 1.00 | 49.55 | N |
| ATOM | 9748 | CA | PRO | B | 237 | 17.528 | 46.266 | 18.364 | 1.00 | 53.89 | C |
| ATOM | 9750 | CB | PRO | B | 237 | 19.037 | 46.236 | 18.036 | 1.00 | 52.34 | C |
| ATOM | 9753 | CG | PRO | B | 237 | 19.634 | 47.291 | 18.888 | 1.00 | 51.34 | C |
| ATOM | 9756 | CD | PRO | B | 237 | 18.837 | 47.302 | 20.146 | 1.00 | 49.53 | C |
| ATOM | 9759 | C | PRO | B | 237 | 17.003 | 44.846 | 18.178 | 1.00 | 55.60 | C |
| ATOM | 9760 | O | PRO | B | 237 | 17.524 | 43.944 | 18.794 | 1.00 | 51.46 | O |
| ATOM | 9761 | N | LYS | B | 238 | 16.027 | 44.660 | 17.297 | 1.00 | 62.69 | N |
| ATOM | 9763 | CA | LYS | B | 238 | 15.321 | 43.386 | 17.149 | 1.00 | 62.37 | C |
| ATOM | 9765 | CB | LYS | B | 238 | 14.847 | 43.178 | 15.702 | 1.00 | 63.90 | C |
| ATOM | 9768 | CG | LYS | B | 238 | 14.017 | 41.850 | 15.476 | 1.00 | 67.48 | C |
| ATOM | 9771 | CD | LYS | B | 238 | 12.652 | 41.826 | 16.191 | 1.00 | 70.44 | C |
| ATOM | 9774 | CE | LYS | B | 238 | 12.375 | 40.445 | 16.872 | 1.00 | 75.03 | C |
| ATOM | 9777 | NZ | LYS | B | 238 | 10.906 | 40.068 | 17.069 | 1.00 | 74.40 | N |
| ATOM | 9781 | C | LYS | B | 238 | 16.015 | 42.134 | 17.728 | 1.00 | 60.78 | C |
| ATOM | 9782 | O | LYS | B | 238 | 15.417 | 41.472 | 18.563 | 1.00 | 63.50 | O |
| ATOM | 9783 | N | LYS | B | 239 | 17.242 | 41.808 | 17.318 | 1.00 | 62.12 | N |
| ATOM | 9785 | CA | LYS | B | 239 | 17.976 | 40.613 | 17.832 | 1.00 | 62.88 | C |
| ATOM | 9787 | CB | LYS | B | 239 | 19.245 | 40.375 | 17.004 | 1.00 | 63.86 | C |
| ATOM | 9790 | CG | LYS | B | 239 | 19.081 | 39.310 | 15.932 | 1.00 | 71.17 | C |
| ATOM | 9793 | CD | LYS | B | 239 | 20.316 | 39.144 | 15.035 | 1.00 | 73.03 | C |
| ATOM | 9796 | CE | LYS | B | 239 | 20.418 | 37.713 | 14.440 | 1.00 | 74.08 | C |
| ATOM | 9799 | NZ | LYS | B | 239 | 20.797 | 37.688 | 12.972 | 1.00 | 69.81 | N |
| ATOM | 9803 | C | LYS | B | 239 | 18.422 | 40.587 | 19.317 | 1.00 | 63.28 | C |
| ATOM | 9804 | O | LYS | B | 239 | 18.587 | 39.508 | 19.896 | 1.00 | 62.95 | O |
| ATOM | 9805 | N | VAL | B | 240 | 18.627 | 41.760 | 19.915 | 1.00 | 58.01 | N |
| ATOM | 9807 | CA | VAL | B | 240 | 19.131 | 41.875 | 21.269 | 1.00 | 54.79 | C |
| ATOM | 9809 | CB | VAL | B | 240 | 19.827 | 43.225 | 21.427 | 1.00 | 58.03 | C |
| ATOM | 9811 | CG1 | VAL | B | 240 | 20.256 | 43.484 | 22.875 | 1.00 | 57.45 | C |
| ATOM | 9815 | CG2 | VAL | B | 240 | 21.047 | 43.292 | 20.450 | 1.00 | 57.36 | C |
| ATOM | 9819 | C | VAL | B | 240 | 17.964 | 41.707 | 22.220 | 1.00 | 56.00 | C |
| ATOM | 9820 | O | VAL | B | 240 | 17.980 | 40.890 | 23.143 | 1.00 | 52.22 | O |
| ATOM | 9821 | N | PHE | B | 241 | 16.928 | 42.488 | 22.012 | 1.00 | 55.23 | N |
| ATOM | 9823 | CA | PHE | B | 241 | 15.725 | 42.212 | 22.742 | 1.00 | 53.80 | C |
| ATOM | 9825 | CB | PHE | B | 241 | 14.545 | 42.796 | 22.032 | 1.00 | 51.30 | C |
| ATOM | 9828 | CG | PHE | B | 241 | 13.300 | 42.770 | 22.822 | 1.00 | 47.65 | C |
| ATOM | 9829 | CD1 | PHE | B | 241 | 13.154 | 43.590 | 23.937 | 1.00 | 48.68 | C |
| ATOM | 9831 | CE1 | PHE | B | 241 | 11.997 | 43.588 | 24.677 | 1.00 | 50.08 | C |
| ATOM | 9833 | CZ | PHE | B | 241 | 10.952 | 42.755 | 24.315 | 1.00 | 51.83 | C |
| ATOM | 9835 | CE2 | PHE | B | 241 | 11.085 | 41.930 | 23.193 | 1.00 | 54.34 | C |
| ATOM | 9837 | CD2 | PHE | B | 241 | 12.267 | 41.953 | 22.451 | 1.00 | 52.82 | C |
| ATOM | 9839 | C | PHE | B | 241 | 15.546 | 40.714 | 22.793 | 1.00 | 55.05 | C |
| ATOM | 9840 | O | PHE | B | 241 | 15.660 | 40.121 | 23.852 | 1.00 | 58.72 | O |
| ATOM | 9841 | N | GLU | B | 242 | 15.296 | 40.080 | 21.651 | 1.00 | 58.90 | N |
| ATOM | 9843 | CA | GLU | B | 242 | 14.915 | 38.650 | 21.665 | 1.00 | 61.77 | C |
| ATOM | 9845 | CB | GLU | B | 242 | 14.771 | 38.033 | 20.255 | 1.00 | 64.23 | C |
| ATOM | 9848 | CG | GLU | B | 242 | 13.434 | 38.214 | 19.518 | 1.00 | 66.72 | C |
| ATOM | 9851 | CD | GLU | B | 242 | 12.187 | 37.837 | 20.319 | 1.00 | 73.25 | C |
| ATOM | 9852 | OE1 | GLU | B | 242 | 11.766 | 36.659 | 20.267 | 1.00 | 74.25 | O |
| ATOM | 9853 | OE2 | GLU | B | 242 | 11.605 | 38.724 | 20.997 | 1.00 | 80.65 | O |
| ATOM | 9854 | C | GLU | B | 242 | 15.946 | 37.859 | 22.473 | 1.00 | 60.51 | C |
| ATOM | 9855 | O | GLU | B | 242 | 15.606 | 37.063 | 23.317 | 1.00 | 61.16 | O |
| ATOM | 9856 | N | ALA | B | 243 | 17.221 | 38.094 | 22.222 | 1.00 | 59.55 | N |
| ATOM | 9858 | CA | ALA | B | 243 | 18.246 | 37.412 | 22.981 | 1.00 | 54.76 | C |
| ATOM | 9860 | CB | ALA | B | 243 | 19.628 | 37.910 | 22.546 | 1.00 | 50.14 | C |
| ATOM | 9864 | C | ALA | B | 243 | 18.056 | 37.628 | 24.480 | 1.00 | 57.05 | C |
| ATOM | 9865 | O | ALA | B | 243 | 18.157 | 36.691 | 25.272 | 1.00 | 60.16 | O |
| ATOM | 9866 | N | ALA | B | 244 | 17.764 | 38.868 | 24.872 | 1.00 | 60.67 | N |
| ATOM | 9868 | CA | ALA | B | 244 | 17.847 | 39.252 | 26.277 | 1.00 | 56.99 | C |
| ATOM | 9870 | CB | ALA | B | 244 | 17.983 | 40.721 | 26.396 | 1.00 | 54.73 | C |
| ATOM | 9874 | C | ALA | B | 244 | 16.670 | 38.756 | 27.039 | 1.00 | 57.52 | C |
| ATOM | 9875 | O | ALA | B | 244 | 16.814 | 38.027 | 28.022 | 1.00 | 66.61 | O |
| ATOM | 9876 | N | VAL | B | 245 | 15.497 | 39.121 | 26.578 | 1.00 | 58.39 | N |
| ATOM | 9878 | CA | VAL | B | 245 | 14.264 | 38.628 | 27.182 | 1.00 | 57.61 | C |
| ATOM | 9880 | CB | VAL | B | 245 | 13.084 | 38.947 | 26.272 | 1.00 | 55.77 | C |
| ATOM | 9882 | CG1 | VAL | B | 245 | 11.921 | 38.046 | 26.582 | 1.00 | 60.16 | C |
| ATOM | 9886 | CG2 | VAL | B | 245 | 12.682 | 40.395 | 26.425 | 1.00 | 55.68 | C |
| ATOM | 9890 | C | VAL | B | 245 | 14.276 | 37.104 | 27.445 | 1.00 | 56.76 | C |
| ATOM | 9891 | O | VAL | B | 245 | 13.735 | 36.630 | 28.457 | 1.00 | 58.98 | O |
| ATOM | 9892 | N | LYS | B | 246 | 14.863 | 36.289 | 26.581 | 1.00 | 54.47 | N |
| ATOM | 9894 | CA | LYS | B | 246 | 14.782 | 34.895 | 26.954 | 1.00 | 59.19 | C |
| ATOM | 9896 | CB | LYS | B | 246 | 15.091 | 33.852 | 25.848 | 1.00 | 58.81 | C |
| ATOM | 9899 | CG | LYS | B | 246 | 16.412 | 33.840 | 25.110 | 1.00 | 66.67 | C |
| ATOM | 9902 | CD | LYS | B | 246 | 16.771 | 32.349 | 24.732 | 1.00 | 73.69 | C |
| ATOM | 9905 | CE | LYS | B | 246 | 17.214 | 32.133 | 23.273 | 1.00 | 77.68 | C |
| ATOM | 9908 | NZ | LYS | B | 246 | 17.712 | 30.722 | 23.053 | 1.00 | 78.54 | N |
| ATOM | 9912 | C | LYS | B | 246 | 15.610 | 34.750 | 28.221 | 1.00 | 60.14 | C |
| ATOM | 9913 | O | LYS | B | 246 | 15.229 | 34.049 | 29.153 | 1.00 | 59.34 | O |
| ATOM | 9914 | N | SER | B | 247 | 16.715 | 35.484 | 28.265 | 1.00 | 59.55 | N |
| ATOM | 9916 | CA | SER | B | 247 | 17.663 | 35.354 | 29.355 | 1.00 | 58.72 | C |
| ATOM | 9918 | CB | SER | B | 247 | 18.952 | 36.101 | 29.017 | 1.00 | 57.44 | C |
| ATOM | 9921 | OG | SER | B | 247 | 20.044 | 35.581 | 29.739 | 1.00 | 59.12 | O |
| ATOM | 9923 | C | SER | B | 247 | 17.082 | 35.830 | 30.676 | 1.00 | 58.15 | C |
| ATOM | 9924 | O | SER | B | 247 | 17.282 | 35.207 | 31.709 | 1.00 | 61.22 | O |
| ATOM | 9925 | N | ILE | B | 248 | 16.339 | 36.915 | 30.632 | 1.00 | 55.00 | N |
| ATOM | 9927 | CA | ILE | B | 248 | 15.793 | 37.501 | 31.837 | 1.00 | 53.84 | C |
| ATOM | 9929 | CB | ILE | B | 248 | 15.123 | 38.845 | 31.474 | 1.00 | 53.06 | C |
| ATOM | 9931 | CG1 | ILE | B | 248 | 16.196 | 39.788 | 30.939 | 1.00 | 51.58 | C |
| ATOM | 9934 | CD1 | ILE | B | 248 | 15.656 | 41.103 | 30.457 | 1.00 | 55.81 | C |
| ATOM | 9938 | CG2 | ILE | B | 248 | 14.381 | 39.461 | 32.661 | 1.00 | 52.52 | C |
| ATOM | 9942 | C | ILE | B | 248 | 14.821 | 36.536 | 32.460 | 1.00 | 54.27 | C |
| ATOM | 9943 | O | ILE | B | 248 | 14.720 | 36.477 | 33.668 | 1.00 | 51.87 | O |
| ATOM | 9944 | N | LYS | B | 249 | 14.114 | 35.784 | 31.619 | 1.00 | 58.95 | N |
| ATOM | 9946 | CA | LYS | B | 249 | 13.080 | 34.863 | 32.076 | 1.00 | 63.35 | C |
| ATOM | 9948 | CB | LYS | B | 249 | 12.386 | 34.163 | 30.895 | 1.00 | 67.38 | C |
| ATOM | 9951 | CG | LYS | B | 249 | 11.566 | 35.076 | 29.969 | 1.00 | 71.83 | C |
| ATOM | 9954 | CD | LYS | B | 249 | 10.893 | 34.193 | 28.921 | 1.00 | 72.34 | C |
| ATOM | 9957 | CE | LYS | B | 249 | 10.025 | 34.974 | 27.965 | 1.00 | 74.28 | C |
| ATOM | 9960 | NZ | LYS | B | 249 | 10.188 | 34.448 | 26.585 | 1.00 | 75.13 | N |
| ATOM | 9964 | C | LYS | B | 249 | 13.828 | 33.829 | 32.836 | 1.00 | 64.05 | C |
| ATOM | 9965 | O | LYS | B | 249 | 13.596 | 33.579 | 34.028 | 1.00 | 66.66 | O |
| ATOM | 9966 | N | ALA | B | 250 | 14.755 | 33.235 | 32.098 | 1.00 | 60.44 | N |
| ATOM | 9968 | CA | ALA | B | 250 | 15.572 | 32.211 | 32.626 | 1.00 | 52.39 | C |
| ATOM | 9970 | CB | ALA | B | 250 | 16.787 | 32.061 | 31.812 | 1.00 | 47.35 | C |
| ATOM | 9974 | C | ALA | B | 250 | 15.897 | 32.687 | 33.993 | 1.00 | 53.08 | C |
| ATOM | 9975 | O | ALA | B | 250 | 15.492 | 32.096 | 34.986 | 1.00 | 60.98 | O |
| ATOM | 9976 | N | ALA | B | 251 | 16.602 | 33.792 | 34.071 | 1.00 | 55.04 | N |
| ATOM | 9978 | CA | ALA | B | 251 | 17.098 | 34.209 | 35.367 | 1.00 | 52.66 | C |
| ATOM | 9980 | CB | ALA | B | 251 | 17.817 | 35.549 | 35.273 | 1.00 | 50.20 | C |
| ATOM | 9984 | C | ALA | B | 251 | 15.943 | 34.276 | 36.333 | 1.00 | 52.91 | C |
| ATOM | 9985 | O | ALA | B | 251 | 16.084 | 33.908 | 37.476 | 1.00 | 63.90 | O |
| ATOM | 9986 | N | SER | B | 252 | 14.786 | 34.706 | 35.865 | 1.00 | 51.39 | N |
| ATOM | 9988 | CA | SER | B | 252 | 13.655 | 34.931 | 36.738 | 1.00 | 47.50 | C |
| ATOM | 9990 | CB | SER | B | 252 | 12.838 | 36.112 | 36.195 | 1.00 | 45.09 | C |
| ATOM | 9993 | OG | SER | B | 252 | 12.937 | 36.176 | 34.777 | 1.00 | 44.57 | O |
| ATOM | 9995 | C | SER | B | 252 | 12.744 | 33.741 | 36.858 | 1.00 | 46.38 | C |
| ATOM | 9996 | O | SER | B | 252 | 11.696 | 33.817 | 37.506 | 1.00 | 52.86 | O |
| ATOM | 9997 | N | SER | B | 253 | 13.095 | 32.626 | 36.257 | 1.00 | 46.89 | N |
| ATOM | 9999 | CA | SER | B | 253 | 12.113 | 31.515 | 36.239 | 1.00 | 51.70 | C |
| ATOM | 10001 | CB | SER | B | 253 | 12.694 | 30.258 | 35.673 | 1.00 | 49.29 | C |
| ATOM | 10004 | OG | SER | B | 253 | 12.622 | 29.350 | 36.744 | 1.00 | 50.36 | O |
| ATOM | 10006 | C | SER | B | 253 | 11.493 | 31.064 | 37.584 | 1.00 | 50.23 | C |
| ATOM | 10007 | O | SER | B | 253 | 10.549 | 30.335 | 37.570 | 1.00 | 48.06 | O |
| ATOM | 10008 | N | THR | B | 254 | 11.992 | 31.448 | 38.743 | 1.00 | 54.64 | N |
| ATOM | 10010 | CA | THR | B | 254 | 11.303 | 30.979 | 39.944 | 1.00 | 55.86 | C |
| ATOM | 10012 | CB | THR | B | 254 | 11.876 | 31.518 | 41.285 | 1.00 | 55.22 | C |
| ATOM | 10014 | OG1 | THR | B | 254 | 12.383 | 32.851 | 41.131 | 1.00 | 59.23 | O |
| ATOM | 10016 | CG2 | THR | B | 254 | 13.081 | 30.702 | 41.690 | 1.00 | 57.98 | C |
| ATOM | 10020 | C | THR | B | 254 | 9.853 | 31.304 | 39.850 | 1.00 | 56.31 | C |
| ATOM | 10021 | O | THR | B | 254 | 9.067 | 30.740 | 40.587 | 1.00 | 58.63 | O |
| ATOM | 10022 | N | GLU | B | 255 | 9.496 | 32.222 | 38.954 | 1.00 | 63.22 | N |
| ATOM | 10024 | CA | GLU | B | 255 | 8.081 | 32.520 | 38.661 | 1.00 | 64.71 | C |
| ATOM | 10026 | CB | GLU | B | 255 | 7.691 | 33.884 | 39.210 | 1.00 | 64.18 | C |
| ATOM | 10029 | CG | GLU | B | 255 | 7.814 | 33.921 | 40.721 | 1.00 | 65.62 | C |
| ATOM | 10032 | CD | GLU | B | 255 | 6.988 | 35.013 | 41.364 | 1.00 | 65.81 | C |
| ATOM | 10033 | OE1 | GLU | B | 255 | 5.941 | 35.412 | 40.801 | 1.00 | 65.88 | O |
| ATOM | 10034 | OE2 | GLU | B | 255 | 7.398 | 35.458 | 42.452 | 1.00 | 67.44 | O |
| ATOM | 10035 | C | GLU | B | 255 | 7.843 | 32.474 | 37.155 | 1.00 | 65.92 | C |
| ATOM | 10036 | O | GLU | B | 255 | 8.761 | 32.231 | 36.374 | 1.00 | 67.96 | O |
| ATOM | 10037 | N | LYS | B | 256 | 6.611 | 32.686 | 36.737 | 1.00 | 64.15 | N |
| ATOM | 10039 | CA | LYS | B | 256 | 6.347 | 32.771 | 35.319 | 1.00 | 62.25 | C |
| ATOM | 10041 | CB | LYS | B | 256 | 5.753 | 31.487 | 34.780 | 1.00 | 65.89 | C |
| ATOM | 10044 | CG | LYS | B | 256 | 6.795 | 30.358 | 34.631 | 1.00 | 70.21 | C |
| ATOM | 10047 | CD | LYS | B | 256 | 7.580 | 30.472 | 33.304 | 1.00 | 71.31 | C |
| ATOM | 10050 | CE | LYS | B | 256 | 8.688 | 29.418 | 33.197 | 1.00 | 70.80 | C |
| ATOM | 10053 | NZ | LYS | B | 256 | 9.037 | 28.832 | 34.535 | 1.00 | 71.41 | N |
| ATOM | 10057 | C | LYS | B | 256 | 5.414 | 33.918 | 35.091 | 1.00 | 61.03 | C |
| ATOM | 10058 | O | LYS | B | 256 | 4.620 | 34.277 | 35.977 | 1.00 | 57.24 | O |
| ATOM | 10059 | N | PHE | B | 257 | 5.522 | 34.500 | 33.903 | 1.00 | 57.37 | N |
| ATOM | 10061 | CA | PHE | B | 257 | 4.700 | 35.632 | 33.563 | 1.00 | 58.14 | C |
| ATOM | 10063 | CB | PHE | B | 257 | 5.557 | 36.904 | 33.635 | 1.00 | 57.69 | C |
| ATOM | 10066 | CG | PHE | B | 257 | 6.262 | 37.095 | 34.971 | 1.00 | 52.26 | C |
| ATOM | 10067 | CD1 | PHE | B | 257 | 7.620 | 36.875 | 35.098 | 1.00 | 55.74 | C |
| ATOM | 10069 | CE1 | PHE | B | 257 | 8.248 | 37.044 | 36.298 | 1.00 | 55.86 | C |
| ATOM | 10071 | CZ | PHE | B | 257 | 7.530 | 37.444 | 37.391 | 1.00 | 56.14 | C |
| ATOM | 10073 | CE2 | PHE | B | 257 | 6.194 | 37.666 | 37.274 | 1.00 | 54.82 | C |
| ATOM | 10075 | CD2 | PHE | B | 257 | 5.569 | 37.490 | 36.073 | 1.00 | 49.91 | C |
| ATOM | 10077 | C | PHE | B | 257 | 4.089 | 35.397 | 32.177 | 1.00 | 61.13 | C |
| ATOM | 10078 | O | PHE | B | 257 | 4.687 | 34.705 | 31.356 | 1.00 | 60.35 | O |
| ATOM | 10079 | N | PRO | B | 258 | 2.902 | 35.964 | 31.934 | 1.00 | 64.44 | N |
| ATOM | 10080 | CA | PRO | B | 258 | 2.175 | 35.834 | 30.651 | 1.00 | 67.15 | C |
| ATOM | 10082 | CB | PRO | B | 258 | 0.765 | 36.286 | 31.018 | 1.00 | 65.96 | C |
| ATOM | 10085 | CG | PRO | B | 258 | 0.946 | 37.246 | 32.161 | 1.00 | 63.58 | C |
| ATOM | 10088 | CD | PRO | B | 258 | 2.172 | 36.804 | 32.906 | 1.00 | 63.13 | C |
| ATOM | 10091 | C | PRO | B | 258 | 2.740 | 36.758 | 29.561 | 1.00 | 71.79 | C |
| ATOM | 10092 | O | PRO | B | 258 | 2.885 | 37.952 | 29.830 | 1.00 | 76.81 | O |
| ATOM | 10093 | N | ASP | B | 259 | 3.010 | 36.250 | 28.357 | 1.00 | 73.38 | N |
| ATOM | 10095 | CA | ASP | B | 259 | 3.820 | 36.980 | 27.356 | 1.00 | 75.86 | C |
| ATOM | 10097 | CB | ASP | B | 259 | 3.890 | 36.221 | 26.029 | 1.00 | 77.62 | C |
| ATOM | 10100 | CG | ASP | B | 259 | 3.803 | 34.701 | 26.222 | 1.00 | 78.97 | C |
| ATOM | 10101 | OD1 | ASP | B | 259 | 4.731 | 33.953 | 25.816 | 1.00 | 64.34 | O |
| ATOM | 10102 | OD2 | ASP | B | 259 | 2.814 | 34.178 | 26.786 | 1.00 | 80.20 | O |
| ATOM | 10103 | C | ASP | B | 259 | 3.443 | 38.429 | 27.102 | 1.00 | 76.48 | C |
| ATOM | 10104 | O | ASP | B | 259 | 4.216 | 39.183 | 26.500 | 1.00 | 74.95 | O |
| ATOM | 10105 | N | GLY | B | 260 | 2.259 | 38.822 | 27.548 | 1.00 | 75.19 | N |
| ATOM | 10107 | CA | GLY | B | 260 | 1.905 | 40.221 | 27.489 | 1.00 | 72.78 | C |
| ATOM | 10110 | C | GLY | B | 260 | 2.926 | 40.966 | 28.316 | 1.00 | 68.86 | C |
| ATOM | 10111 | O | GLY | B | 260 | 3.429 | 41.998 | 27.885 | 1.00 | 70.54 | O |
| ATOM | 10112 | N | PHE | B | 261 | 3.239 | 40.425 | 29.495 | 1.00 | 65.94 | N |
| ATOM | 10114 | CA | PHE | B | 261 | 4.187 | 41.062 | 30.408 | 1.00 | 61.43 | C |
| ATOM | 10116 | CB | PHE | B | 261 | 4.317 | 40.311 | 31.709 | 1.00 | 56.82 | C |
| ATOM | 10119 | CG | PHE | B | 261 | 5.391 | 40.871 | 32.570 | 1.00 | 54.73 | C |
| ATOM | 10120 | CD1 | PHE | B | 261 | 5.126 | 41.944 | 33.382 | 1.00 | 51.57 | C |
| ATOM | 10122 | CE1 | PHE | B | 261 | 6.106 | 42.488 | 34.188 | 1.00 | 51.52 | C |
| ATOM | 10124 | CZ | PHE | B | 261 | 7.365 | 41.961 | 34.166 | 1.00 | 57.72 | C |
| ATOM | 10126 | CE2 | PHE | B | 261 | 7.658 | 40.876 | 33.345 | 1.00 | 57.74 | C |
| ATOM | 10128 | CD2 | PHE | B | 261 | 6.676 | 40.343 | 32.550 | 1.00 | 59.54 | C |
| ATOM | 10130 | C | PHE | B | 261 | 5.603 | 41.195 | 29.876 | 1.00 | 57.86 | C |
| ATOM | 10131 | O | PHE | B | 261 | 6.386 | 41.981 | 30.377 | 1.00 | 68.09 | O |
| ATOM | 10132 | N | TRP | B | 262 | 5.954 | 40.389 | 28.892 | 1.00 | 56.00 | N |
| ATOM | 10134 | CA | TRP | B | 262 | 7.253 | 40.518 | 28.263 | 1.00 | 56.28 | C |
| ATOM | 10136 | CB | TRP | B | 262 | 7.932 | 39.170 | 28.049 | 1.00 | 54.41 | C |
| ATOM | 10139 | CG | TRP | B | 262 | 8.136 | 38.278 | 29.238 | 1.00 | 55.03 | C |
| ATOM | 10140 | CD1 | TRP | B | 262 | 7.407 | 37.184 | 29.525 | 1.00 | 52.81 | C |
| ATOM | 10142 | NE1 | TRP | B | 262 | 7.866 | 36.578 | 30.669 | 1.00 | 58.02 | N |
| ATOM | 10144 | CE2 | TRP | B | 262 | 8.931 | 37.281 | 31.156 | 1.00 | 56.77 | C |
| ATOM | 10145 | CD2 | TRP | B | 262 | 9.135 | 38.379 | 30.276 | 1.00 | 63.26 | C |
| ATOM | 10146 | CE3 | TRP | B | 262 | 10.180 | 39.274 | 30.552 | 1.00 | 54.90 | C |
| ATOM | 10148 | CZ3 | TRP | B | 262 | 10.970 | 39.046 | 31.685 | 1.00 | 55.79 | C |
| ATOM | 10150 | CH2 | TRP | B | 262 | 10.733 | 37.955 | 32.537 | 1.00 | 54.57 | C |
| ATOM | 10152 | CZ2 | TRP | B | 262 | 9.717 | 37.063 | 32.288 | 1.00 | 53.00 | C |
| ATOM | 10154 | C | TRP | B | 262 | 7.104 | 41.282 | 26.932 | 1.00 | 55.99 | C |
| ATOM | 10155 | O | TRP | B | 262 | 8.062 | 41.510 | 26.231 | 1.00 | 59.38 | O |
| ATOM | 10156 | N | LEU | B | 263 | 5.907 | 41.686 | 26.565 | 1.00 | 57.34 | N |
| ATOM | 10158 | CA | LEU | B | 263 | 5.811 | 42.625 | 25.461 | 1.00 | 55.32 | C |
| ATOM | 10160 | CB | LEU | B | 263 | 4.939 | 42.116 | 24.311 | 1.00 | 55.06 | C |
| ATOM | 10163 | CG | LEU | B | 263 | 5.272 | 40.716 | 23.756 | 1.00 | 50.87 | C |
| ATOM | 10165 | CD1 | LEU | B | 263 | 4.008 | 40.071 | 23.221 | 1.00 | 53.49 | C |
| ATOM | 10169 | CD2 | LEU | B | 263 | 6.315 | 40.711 | 22.680 | 1.00 | 50.09 | C |
| ATOM | 10173 | C | LEU | B | 263 | 5.282 | 43.907 | 26.099 | 1.00 | 57.11 | C |
| ATOM | 10174 | O | LEU | B | 263 | 4.669 | 44.749 | 25.449 | 1.00 | 62.05 | O |
| ATOM | 10175 | N | GLY | B | 264 | 5.520 | 44.039 | 27.400 | 1.00 | 58.91 | N |
| ATOM | 10177 | CA | GLY | B | 264 | 5.227 | 45.268 | 28.129 | 1.00 | 59.68 | C |
| ATOM | 10180 | C | GLY | B | 264 | 3.855 | 45.865 | 27.896 | 1.00 | 59.63 | C |
| ATOM | 10181 | O | GLY | B | 264 | 3.643 | 47.054 | 28.142 | 1.00 | 64.20 | O |
| ATOM | 10182 | N | GLU | B | 265 | 2.918 | 45.042 | 27.441 | 1.00 | 60.98 | N |
| ATOM | 10184 | CA | GLU | B | 265 | 1.547 | 45.480 | 27.189 | 1.00 | 64.98 | C |
| ATOM | 10186 | CB | GLU | B | 265 | 0.878 | 44.484 | 26.249 | 1.00 | 66.91 | C |
| ATOM | 10189 | CG | GLU | B | 265 | 0.967 | 44.902 | 24.795 | 1.00 | 74.60 | C |
| ATOM | 10192 | CD | GLU | B | 265 | 0.904 | 43.720 | 23.835 | 1.00 | 81.19 | C |
| ATOM | 10193 | OE1 | GLU | B | 265 | 1.023 | 43.968 | 22.603 | 1.00 | 80.62 | O |
| ATOM | 10194 | OE2 | GLU | B | 265 | 0.734 | 42.556 | 24.311 | 1.00 | 85.40 | O |
| ATOM | 10195 | C | GLU | B | 265 | 0.674 | 45.595 | 28.433 | 1.00 | 66.06 | C |
| ATOM | 10196 | O | GLU | B | 265 | −0.178 | 46.471 | 28.534 | 1.00 | 67.79 | O |
| ATOM | 10197 | N | GLN | B | 266 | 0.890 | 44.677 | 29.368 | 1.00 | 65.41 | N |
| ATOM | 10199 | CA | GLN | B | 266 | 0.029 | 44.521 | 30.539 | 1.00 | 66.17 | C |
| ATOM | 10201 | CB | GLN | B | 266 | −0.860 | 43.299 | 30.402 | 1.00 | 66.49 | C |
| ATOM | 10204 | CG | GLN | B | 266 | −0.090 | 42.024 | 30.448 | 1.00 | 66.76 | C |
| ATOM | 10207 | CD | GLN | B | 266 | −0.932 | 40.851 | 30.044 | 1.00 | 71.05 | C |
| ATOM | 10208 | OE1 | GLN | B | 266 | −0.926 | 40.453 | 28.881 | 1.00 | 78.94 | O |
| ATOM | 10209 | NE2 | GLN | B | 266 | −1.660 | 40.282 | 30.996 | 1.00 | 65.67 | N |
| ATOM | 10212 | C | GLN | B | 266 | 0.880 | 44.359 | 31.773 | 1.00 | 66.63 | C |
| ATOM | 10213 | O | GLN | B | 266 | 1.998 | 43.881 | 31.714 | 1.00 | 61.37 | O |
| ATOM | 10214 | N | LEU | B | 267 | 0.335 | 44.745 | 32.910 | 1.00 | 68.01 | N |
| ATOM | 10216 | CA | LEU | B | 267 | 1.196 | 44.910 | 34.055 | 1.00 | 66.65 | C |
| ATOM | 10218 | CB | LEU | B | 267 | 0.782 | 46.202 | 34.780 | 1.00 | 64.75 | C |
| ATOM | 10221 | CG | LEU | B | 267 | −0.664 | 46.456 | 35.117 | 1.00 | 61.97 | C |
| ATOM | 10223 | CD1 | LEU | B | 267 | −0.953 | 45.596 | 36.275 | 1.00 | 65.54 | C |
| ATOM | 10227 | CD2 | LEU | B | 267 | −0.883 | 47.886 | 35.487 | 1.00 | 62.67 | C |
| ATOM | 10231 | C | LEU | B | 267 | 1.305 | 43.674 | 34.947 | 1.00 | 66.02 | C |
| ATOM | 10232 | O | LEU | B | 267 | 0.742 | 42.633 | 34.643 | 1.00 | 65.04 | O |
| ATOM | 10233 | N | VAL | B | 268 | 2.062 | 43.779 | 36.031 | 1.00 | 66.18 | N |
| ATOM | 10235 | CA | VAL | B | 268 | 2.201 | 42.665 | 36.966 | 1.00 | 63.11 | C |
| ATOM | 10237 | CB | VAL | B | 268 | 3.526 | 42.035 | 36.844 | 1.00 | 63.47 | C |
| ATOM | 10239 | CG1 | VAL | B | 268 | 3.584 | 40.817 | 37.674 | 1.00 | 65.01 | C |
| ATOM | 10243 | CG2 | VAL | B | 268 | 3.741 | 41.689 | 35.434 | 1.00 | 67.71 | C |
| ATOM | 10247 | C | VAL | B | 268 | 2.089 | 43.146 | 38.376 | 1.00 | 62.57 | C |
| ATOM | 10248 | O | VAL | B | 268 | 2.535 | 44.227 | 38.723 | 1.00 | 64.30 | O |
| ATOM | 10249 | N | CYS | B | 269 | 1.498 | 42.320 | 39.204 | 1.00 | 62.51 | N |
| ATOM | 10251 | CA | CYS | B | 269 | 1.189 | 42.742 | 40.543 | 1.00 | 62.84 | C |
| ATOM | 10253 | CB | CYS | B | 269 | −0.334 | 42.937 | 40.667 | 1.00 | 64.89 | C |
| ATOM | 10256 | SG | CYS | B | 269 | −0.978 | 44.253 | 39.612 | 1.00 | 68.14 | S |
| ATOM | 10257 | C | CYS | B | 269 | 1.677 | 41.778 | 41.610 | 1.00 | 58.60 | C |
| ATOM | 10258 | O | CYS | B | 269 | 1.707 | 40.556 | 41.420 | 1.00 | 57.72 | O |
| ATOM | 10259 | N | TRP | B | 270 | 2.040 | 42.374 | 42.731 | 1.00 | 50.46 | N |
| ATOM | 10261 | CA | TRP | B | 270 | 2.483 | 41.669 | 43.896 | 1.00 | 53.90 | C |
| ATOM | 10263 | CB | TRP | B | 270 | 3.996 | 41.833 | 44.087 | 1.00 | 54.89 | C |
| ATOM | 10266 | CG | TRP | B | 270 | 4.740 | 40.871 | 43.267 | 1.00 | 55.99 | C |
| ATOM | 10267 | CD1 | TRP | B | 270 | 4.829 | 39.543 | 43.480 | 1.00 | 57.11 | C |
| ATOM | 10269 | NE1 | TRP | B | 270 | 5.601 | 38.961 | 42.503 | 1.00 | 61.98 | N |
| ATOM | 10271 | CE2 | TRP | B | 270 | 6.026 | 39.929 | 41.633 | 1.00 | 61.92 | C |
| ATOM | 10272 | CD2 | TRP | B | 270 | 5.500 | 41.145 | 42.081 | 1.00 | 57.92 | C |
| ATOM | 10273 | CE3 | TRP | B | 270 | 5.793 | 42.297 | 41.355 | 1.00 | 53.37 | C |
| ATOM | 10275 | CZ3 | TRP | B | 270 | 6.571 | 42.199 | 40.241 | 1.00 | 53.54 | C |
| ATOM | 10277 | CH2 | TRP | B | 270 | 7.083 | 40.982 | 39.812 | 1.00 | 56.68 | C |
| ATOM | 10279 | CZ2 | TRP | B | 270 | 6.825 | 39.834 | 40.490 | 1.00 | 64.93 | C |
| ATOM | 10281 | C | TRP | B | 270 | 1.753 | 42.293 | 45.048 | 1.00 | 52.99 | C |
| ATOM | 10282 | O | TRP | B | 270 | 1.321 | 43.432 | 44.976 | 1.00 | 52.30 | O |
| ATOM | 10283 | N | GLN | B | 271 | 1.612 | 41.554 | 46.126 | 1.00 | 56.53 | N |
| ATOM | 10285 | CA | GLN | B | 271 | 0.897 | 42.083 | 47.267 | 1.00 | 58.01 | C |
| ATOM | 10287 | CB | GLN | B | 271 | 0.936 | 41.079 | 48.415 | 1.00 | 60.04 | C |
| ATOM | 10290 | CG | GLN | B | 271 | 0.773 | 41.648 | 49.826 | 1.00 | 69.55 | C |
| ATOM | 10293 | CD | GLN | B | 271 | 1.280 | 40.664 | 50.910 | 1.00 | 75.84 | C |
| ATOM | 10294 | OE1 | GLN | B | 271 | 2.450 | 40.268 | 50.900 | 1.00 | 78.40 | O |
| ATOM | 10295 | NE2 | GLN | B | 271 | 0.392 | 40.262 | 51.825 | 1.00 | 80.06 | N |
| ATOM | 10298 | C | GLN | B | 271 | 1.683 | 43.308 | 47.553 | 1.00 | 54.15 | C |
| ATOM | 10299 | O | GLN | B | 271 | 2.781 | 43.454 | 47.050 | 1.00 | 62.98 | O |
| ATOM | 10300 | N | ALA | B | 272 | 1.162 | 44.209 | 48.341 | 1.00 | 54.28 | N |
| ATOM | 10302 | CA | ALA | B | 272 | 1.964 | 45.351 | 48.683 | 1.00 | 55.76 | C |
| ATOM | 10304 | CB | ALA | B | 272 | 1.280 | 46.192 | 49.767 | 1.00 | 54.56 | C |
| ATOM | 10308 | C | ALA | B | 272 | 3.325 | 44.876 | 49.169 | 1.00 | 57.86 | C |
| ATOM | 10309 | O | ALA | B | 272 | 3.497 | 43.739 | 49.579 | 1.00 | 57.62 | O |
| ATOM | 10310 | N | GLY | B | 273 | 4.296 | 45.776 | 49.067 | 1.00 | 64.15 | N |
| ATOM | 10312 | CA | GLY | B | 273 | 5.605 | 45.660 | 49.699 | 1.00 | 62.02 | C |
| ATOM | 10315 | C | GLY | B | 273 | 6.435 | 44.431 | 49.502 | 1.00 | 59.47 | C |
| ATOM | 10316 | O | GLY | B | 273 | 7.544 | 44.376 | 50.003 | 1.00 | 62.91 | O |
| ATOM | 10317 | N | THR | B | 274 | 5.926 | 43.467 | 48.751 | 1.00 | 59.03 | N |
| ATOM | 10319 | CA | THR | B | 274 | 6.529 | 42.155 | 48.713 | 1.00 | 52.35 | C |
| ATOM | 10321 | CB | THR | B | 274 | 5.483 | 41.091 | 49.087 | 1.00 | 58.59 | C |
| ATOM | 10323 | OG1 | THR | B | 274 | 4.574 | 40.872 | 47.988 | 1.00 | 54.95 | O |
| ATOM | 10325 | CG2 | THR | B | 274 | 4.593 | 41.579 | 50.243 | 1.00 | 55.11 | C |
| ATOM | 10329 | C | THR | B | 274 | 6.971 | 41.857 | 47.351 | 1.00 | 46.12 | C |
| ATOM | 10330 | O | THR | B | 274 | 6.961 | 40.726 | 46.957 | 1.00 | 46.68 | O |
| ATOM | 10331 | N | THR | B | 275 | 7.332 | 42.868 | 46.600 | 1.00 | 47.81 | N |
| ATOM | 10333 | CA | THR | B | 275 | 7.866 | 42.606 | 45.291 | 1.00 | 48.64 | C |
| ATOM | 10335 | CB | THR | B | 275 | 8.257 | 43.907 | 44.540 | 1.00 | 47.68 | C |
| ATOM | 10337 | OG1 | THR | B | 275 | 7.090 | 44.596 | 44.064 | 1.00 | 50.08 | O |
| ATOM | 10339 | CG2 | THR | B | 275 | 9.005 | 43.551 | 43.276 | 1.00 | 46.87 | C |
| ATOM | 10343 | C | THR | B | 275 | 9.114 | 41.783 | 45.539 | 1.00 | 51.15 | C |
| ATOM | 10344 | O | THR | B | 275 | 9.984 | 42.143 | 46.371 | 1.00 | 54.79 | O |
| ATOM | 10345 | N | PRO | B | 276 | 9.190 | 40.685 | 44.822 | 1.00 | 46.67 | N |
| ATOM | 10346 | CA | PRO | B | 276 | 10.355 | 39.808 | 44.811 | 1.00 | 48.43 | C |
| ATOM | 10348 | CB | PRO | B | 276 | 9.895 | 38.611 | 43.955 | 1.00 | 50.23 | C |
| ATOM | 10351 | CG | PRO | B | 276 | 8.574 | 38.955 | 43.383 | 1.00 | 51.47 | C |
| ATOM | 10354 | CD | PRO | B | 276 | 8.101 | 40.227 | 43.956 | 1.00 | 50.10 | C |
| ATOM | 10357 | C | PRO | B | 276 | 11.557 | 40.420 | 44.091 | 1.00 | 48.73 | C |
| ATOM | 10358 | O | PRO | B | 276 | 12.010 | 39.796 | 43.088 | 1.00 | 44.55 | O |
| ATOM | 10359 | N | TRP | B | 277 | 12.083 | 41.567 | 44.537 | 1.00 | 48.39 | N |
| ATOM | 10361 | CA | TRP | B | 277 | 13.120 | 42.230 | 43.711 | 1.00 | 48.55 | C |
| ATOM | 10363 | CB | TRP | B | 277 | 13.740 | 43.469 | 44.393 | 1.00 | 48.09 | C |
| ATOM | 10366 | CG | TRP | B | 277 | 12.769 | 44.677 | 44.518 | 1.00 | 50.79 | C |
| ATOM | 10367 | CD1 | TRP | B | 277 | 12.309 | 45.236 | 45.680 | 1.00 | 54.20 | C |
| ATOM | 10369 | NE1 | TRP | B | 277 | 11.463 | 46.285 | 45.412 | 1.00 | 46.92 | N |
| ATOM | 10371 | CE2 | TRP | B | 277 | 11.359 | 46.429 | 44.063 | 1.00 | 42.74 | C |
| ATOM | 10372 | CD2 | TRP | B | 277 | 12.164 | 45.439 | 43.466 | 1.00 | 47.17 | C |
| ATOM | 10373 | CE3 | TRP | B | 277 | 12.222 | 45.390 | 42.078 | 1.00 | 51.68 | C |
| ATOM | 10375 | CZ3 | TRP | B | 277 | 11.486 | 46.318 | 41.352 | 1.00 | 46.11 | C |
| ATOM | 10377 | CH2 | TRP | B | 277 | 10.703 | 47.270 | 41.991 | 1.00 | 45.63 | C |
| ATOM | 10379 | CZ2 | TRP | B | 277 | 10.626 | 47.338 | 43.337 | 1.00 | 42.71 | C |
| ATOM | 10381 | C | TRP | B | 277 | 14.202 | 41.229 | 43.271 | 1.00 | 45.71 | C |
| ATOM | 10382 | O | TRP | B | 277 | 14.664 | 41.180 | 42.123 | 1.00 | 48.22 | O |
| ATOM | 10383 | N | ASN | B | 278 | 14.594 | 40.388 | 44.184 | 1.00 | 50.19 | N |
| ATOM | 10385 | CA | ASN | B | 278 | 15.742 | 39.549 | 43.918 | 1.00 | 48.45 | C |
| ATOM | 10387 | CB | ASN | B | 278 | 16.264 | 39.003 | 45.227 | 1.00 | 53.84 | C |
| ATOM | 10390 | CG | ASN | B | 278 | 15.300 | 38.046 | 45.876 | 1.00 | 53.98 | C |
| ATOM | 10391 | OD1 | ASN | B | 278 | 15.630 | 36.887 | 46.122 | 1.00 | 58.86 | O |
| ATOM | 10392 | ND2 | ASN | B | 278 | 14.095 | 38.528 | 46.162 | 1.00 | 58.00 | N |
| ATOM | 10395 | C | ASN | B | 278 | 15.579 | 38.410 | 42.941 | 1.00 | 47.96 | C |
| ATOM | 10396 | O | ASN | B | 278 | 16.577 | 37.810 | 42.616 | 1.00 | 48.00 | O |
| ATOM | 10397 | N | ILE | B | 279 | 14.384 | 38.074 | 42.447 | 1.00 | 48.44 | N |
| ATOM | 10399 | CA | ILE | B | 279 | 14.356 | 36.960 | 41.496 | 1.00 | 44.44 | C |
| ATOM | 10401 | CB | ILE | B | 279 | 13.006 | 36.227 | 41.370 | 1.00 | 48.45 | C |
| ATOM | 10403 | CG1 | ILE | B | 279 | 11.844 | 37.181 | 41.143 | 1.00 | 53.39 | C |
| ATOM | 10406 | CD1 | ILE | B | 279 | 10.671 | 36.467 | 40.444 | 1.00 | 49.91 | C |
| ATOM | 10410 | CG2 | ILE | B | 279 | 12.724 | 35.335 | 42.562 | 1.00 | 44.38 | C |
| ATOM | 10414 | C | ILE | B | 279 | 14.796 | 37.386 | 40.131 | 1.00 | 47.23 | C |
| ATOM | 10415 | O | ILE | B | 279 | 15.121 | 36.515 | 39.311 | 1.00 | 48.85 | O |
| ATOM | 10416 | N | PHE | B | 280 | 14.809 | 38.714 | 39.886 | 1.00 | 51.85 | N |
| ATOM | 10418 | CA | PHE | B | 280 | 15.181 | 39.313 | 38.572 | 1.00 | 43.99 | C |
| ATOM | 10420 | CB | PHE | B | 280 | 14.346 | 40.540 | 38.196 | 1.00 | 47.60 | C |
| ATOM | 10423 | CG | PHE | B | 280 | 12.840 | 40.323 | 38.213 | 1.00 | 50.69 | C |
| ATOM | 10424 | CD1 | PHE | B | 280 | 12.157 | 40.062 | 37.040 | 1.00 | 47.59 | C |
| ATOM | 10426 | CE1 | PHE | B | 280 | 10.793 | 39.871 | 37.045 | 1.00 | 49.69 | C |
| ATOM | 10428 | CZ | PHE | B | 280 | 10.114 | 39.944 | 38.220 | 1.00 | 53.43 | C |
| ATOM | 10430 | CE2 | PHE | B | 280 | 10.792 | 40.216 | 39.402 | 1.00 | 50.88 | C |
| ATOM | 10432 | CD2 | PHE | B | 280 | 12.126 | 40.407 | 39.395 | 1.00 | 44.76 | C |
| ATOM | 10434 | C | PHE | B | 280 | 16.604 | 39.788 | 38.614 | 1.00 | 43.94 | C |
| ATOM | 10435 | O | PHE | B | 280 | 17.114 | 40.282 | 39.655 | 1.00 | 43.28 | O |
| ATOM | 10436 | N | PRO | B | 281 | 17.256 | 39.685 | 37.473 | 1.00 | 37.73 | N |
| ATOM | 10437 | CA | PRO | B | 281 | 18.668 | 39.942 | 37.413 | 1.00 | 33.44 | C |
| ATOM | 10439 | CB | PRO | B | 281 | 19.089 | 39.028 | 36.294 | 1.00 | 36.50 | C |
| ATOM | 10442 | CG | PRO | B | 281 | 18.034 | 39.215 | 35.300 | 1.00 | 38.20 | C |
| ATOM | 10445 | CD | PRO | B | 281 | 16.737 | 39.358 | 36.132 | 1.00 | 39.79 | C |
| ATOM | 10448 | C | PRO | B | 281 | 18.928 | 41.346 | 37.036 | 1.00 | 36.85 | C |
| ATOM | 10449 | O | PRO | B | 281 | 18.039 | 41.984 | 36.537 | 1.00 | 43.97 | O |
| ATOM | 10450 | N | VAL | B | 282 | 20.144 | 41.806 | 37.290 | 1.00 | 42.26 | N |
| ATOM | 10452 | CA | VAL | B | 282 | 20.585 | 43.141 | 36.983 | 1.00 | 39.23 | C |
| ATOM | 10454 | CB | VAL | B | 282 | 21.818 | 43.477 | 37.809 | 1.00 | 43.53 | C |
| ATOM | 10456 | CG1 | VAL | B | 282 | 21.497 | 43.457 | 39.275 | 1.00 | 40.33 | C |
| ATOM | 10460 | CG2 | VAL | B | 282 | 22.988 | 42.465 | 37.444 | 1.00 | 39.80 | C |
| ATOM | 10464 | C | VAL | B | 282 | 21.100 | 43.067 | 35.569 | 1.00 | 41.69 | C |
| ATOM | 10465 | O | VAL | B | 282 | 21.582 | 42.011 | 35.184 | 1.00 | 35.21 | O |
| ATOM | 10466 | N | ILE | B | 283 | 21.025 | 44.169 | 34.819 | 1.00 | 39.90 | N |
| ATOM | 10468 | CA | ILE | B | 283 | 21.558 | 44.219 | 33.457 | 1.00 | 39.37 | C |
| ATOM | 10470 | CB | ILE | B | 283 | 20.505 | 44.748 | 32.505 | 1.00 | 40.29 | C |
| ATOM | 10472 | CG1 | ILE | B | 283 | 19.273 | 43.855 | 32.531 | 1.00 | 35.95 | C |
| ATOM | 10475 | CD1 | ILE | B | 283 | 18.066 | 44.572 | 32.090 | 1.00 | 41.11 | C |
| ATOM | 10479 | CG2 | ILE | B | 283 | 21.074 | 44.867 | 31.102 | 1.00 | 33.71 | C |
| ATOM | 10483 | C | ILE | B | 283 | 22.709 | 45.198 | 33.425 | 1.00 | 46.04 | C |
| ATOM | 10484 | O | ILE | B | 283 | 22.691 | 46.233 | 34.110 | 1.00 | 49.32 | O |
| ATOM | 10485 | N | SER | B | 284 | 23.709 | 44.885 | 32.612 | 1.00 | 48.26 | N |
| ATOM | 10487 | CA | SER | B | 284 | 24.893 | 45.713 | 32.522 | 1.00 | 46.53 | C |
| ATOM | 10489 | CB | SER | B | 284 | 26.088 | 44.939 | 33.112 | 1.00 | 44.99 | C |
| ATOM | 10492 | OG | SER | B | 284 | 25.927 | 44.696 | 34.522 | 1.00 | 44.94 | O |
| ATOM | 10494 | C | SER | B | 284 | 25.206 | 46.048 | 31.086 | 1.00 | 46.23 | C |
| ATOM | 10495 | O | SER | B | 284 | 25.284 | 45.130 | 30.269 | 1.00 | 51.09 | O |
| ATOM | 10496 | N | LEU | B | 285 | 25.388 | 47.325 | 30.764 | 1.00 | 41.48 | N |
| ATOM | 10498 | CA | LEU | B | 285 | 25.877 | 47.669 | 29.447 | 1.00 | 39.92 | C |
| ATOM | 10500 | CB | LEU | B | 285 | 24.939 | 48.679 | 28.756 | 1.00 | 35.47 | C |
| ATOM | 10503 | CG | LEU | B | 285 | 23.394 | 48.570 | 28.921 | 1.00 | 38.05 | C |
| ATOM | 10505 | CD1 | LEU | B | 285 | 22.708 | 49.787 | 28.300 | 1.00 | 35.50 | C |
| ATOM | 10509 | CD2 | LEU | B | 285 | 22.717 | 47.308 | 28.356 | 1.00 | 37.92 | C |
| ATOM | 10513 | C | LEU | B | 285 | 27.330 | 48.156 | 29.671 | 1.00 | 39.56 | C |
| ATOM | 10514 | O | LEU | B | 285 | 27.562 | 48.901 | 30.617 | 1.00 | 37.44 | O |
| ATOM | 10515 | N | TYR | B | 286 | 28.288 | 47.684 | 28.850 | 1.00 | 39.99 | N |
| ATOM | 10517 | CA | TYR | B | 286 | 29.697 | 48.167 | 28.835 | 1.00 | 45.00 | C |
| ATOM | 10519 | CB | TYR | B | 286 | 30.698 | 47.012 | 28.685 | 1.00 | 46.18 | C |
| ATOM | 10522 | CG | TYR | B | 286 | 30.744 | 45.888 | 29.752 | 1.00 | 48.12 | C |
| ATOM | 10523 | CD1 | TYR | B | 286 | 29.678 | 45.025 | 29.975 | 1.00 | 47.49 | C |
| ATOM | 10525 | CE1 | TYR | B | 286 | 29.767 | 44.036 | 30.934 | 1.00 | 46.99 | C |
| ATOM | 10527 | CZ | TYR | B | 286 | 30.945 | 43.911 | 31.667 | 1.00 | 49.87 | C |
| ATOM | 10528 | OH | TYR | B | 286 | 31.134 | 42.952 | 32.643 | 1.00 | 45.54 | O |
| ATOM | 10530 | CE2 | TYR | B | 286 | 31.986 | 44.753 | 31.444 | 1.00 | 45.24 | C |
| ATOM | 10532 | CD2 | TYR | B | 286 | 31.892 | 45.699 | 30.511 | 1.00 | 38.43 | C |
| ATOM | 10534 | C | TYR | B | 286 | 29.917 | 49.065 | 27.602 | 1.00 | 46.95 | C |
| ATOM | 10535 | O | TYR | B | 286 | 29.613 | 48.633 | 26.494 | 1.00 | 43.90 | O |
| ATOM | 10536 | N | LEU | B | 287 | 30.480 | 50.269 | 27.782 | 1.00 | 42.38 | N |
| ATOM | 10538 | CA | LEU | B | 287 | 30.526 | 51.257 | 26.741 | 1.00 | 40.41 | C |
| ATOM | 10540 | CB | LEU | B | 287 | 29.874 | 52.562 | 27.203 | 1.00 | 39.65 | C |
| ATOM | 10543 | CG | LEU | B | 287 | 28.487 | 52.664 | 27.896 | 1.00 | 35.23 | C |
| ATOM | 10545 | CD1 | LEU | B | 287 | 28.344 | 53.994 | 28.625 | 1.00 | 37.04 | C |
| ATOM | 10549 | CD2 | LEU | B | 287 | 27.480 | 52.558 | 26.938 | 1.00 | 26.22 | C |
| ATOM | 10553 | C | LEU | B | 287 | 31.960 | 51.571 | 26.564 | 1.00 | 45.87 | C |
| ATOM | 10554 | O | LEU | B | 287 | 32.716 | 51.400 | 27.474 | 1.00 | 51.50 | O |
| ATOM | 10555 | N | MET | B | 288 | 32.356 | 52.041 | 25.401 | 1.00 | 50.25 | N |
| ATOM | 10557 | CA | MET | B | 288 | 33.749 | 52.315 | 25.123 | 1.00 | 52.89 | C |
| ATOM | 10559 | CB | MET | B | 288 | 33.884 | 52.619 | 23.654 | 1.00 | 57.49 | C |
| ATOM | 10562 | CG | MET | B | 288 | 35.255 | 52.585 | 23.075 | 1.00 | 59.46 | C |
| ATOM | 10565 | SD | MET | B | 288 | 35.108 | 53.029 | 21.308 | 1.00 | 62.39 | S |
| ATOM | 10566 | CE | MET | B | 288 | 35.316 | 51.616 | 20.631 | 1.00 | 56.14 | C |
| ATOM | 10570 | C | MET | B | 288 | 34.157 | 53.518 | 25.927 | 1.00 | 55.91 | C |
| ATOM | 10571 | O | MET | B | 288 | 33.354 | 54.433 | 26.109 | 1.00 | 57.24 | O |
| ATOM | 10572 | N | GLY | B | 289 | 35.403 | 53.522 | 26.405 | 1.00 | 56.64 | N |
| ATOM | 10574 | CA | GLY | B | 289 | 35.911 | 54.608 | 27.230 | 1.00 | 55.82 | C |
| ATOM | 10577 | C | GLY | B | 289 | 36.880 | 55.482 | 26.473 | 1.00 | 58.37 | C |
| ATOM | 10578 | O | GLY | B | 289 | 37.113 | 55.267 | 25.286 | 1.00 | 59.88 | O |
| ATOM | 10579 | N | GLU | B | 290 | 37.460 | 56.465 | 27.143 | 1.00 | 57.96 | N |
| ATOM | 10581 | CA | GLU | B | 290 | 38.341 | 57.390 | 26.444 | 1.00 | 59.77 | C |
| ATOM | 10583 | CB | GLU | B | 290 | 38.373 | 58.726 | 27.150 | 1.00 | 59.36 | C |
| ATOM | 10586 | CG | GLU | B | 290 | 37.021 | 59.394 | 27.100 | 1.00 | 60.99 | C |
| ATOM | 10589 | CD | GLU | B | 290 | 37.125 | 60.824 | 26.612 | 1.00 | 63.85 | C |
| ATOM | 10590 | OE1 | GLU | B | 290 | 37.333 | 61.687 | 27.500 | 1.00 | 67.58 | O |
| ATOM | 10591 | OE2 | GLU | B | 290 | 37.006 | 61.078 | 25.360 | 1.00 | 50.86 | O |
| ATOM | 10592 | C | GLU | B | 290 | 39.767 | 56.919 | 26.220 | 1.00 | 61.97 | C |
| ATOM | 10593 | O | GLU | B | 290 | 40.377 | 57.319 | 25.243 | 1.00 | 65.39 | O |
| ATOM | 10594 | N | VAL | B | 291 | 40.325 | 56.086 | 27.090 | 1.00 | 62.97 | N |
| ATOM | 10596 | CA | VAL | B | 291 | 41.697 | 55.643 | 26.854 | 1.00 | 61.83 | C |
| ATOM | 10598 | CB | VAL | B | 291 | 42.336 | 55.119 | 28.158 | 1.00 | 63.23 | C |
| ATOM | 10600 | CG1 | VAL | B | 291 | 43.820 | 54.897 | 27.988 | 1.00 | 67.30 | C |
| ATOM | 10604 | CG2 | VAL | B | 291 | 42.121 | 56.153 | 29.281 | 1.00 | 66.27 | C |
| ATOM | 10608 | C | VAL | B | 291 | 41.452 | 54.644 | 25.731 | 1.00 | 59.22 | C |
| ATOM | 10609 | O | VAL | B | 291 | 40.303 | 54.325 | 25.463 | 1.00 | 56.93 | O |
| ATOM | 10610 | N | THR | B | 292 | 42.468 | 54.169 | 25.038 | 1.00 | 58.29 | N |
| ATOM | 10612 | CA | THR | B | 292 | 42.227 | 53.183 | 23.969 | 1.00 | 59.67 | C |
| ATOM | 10614 | CB | THR | B | 292 | 43.311 | 53.327 | 22.909 | 1.00 | 62.91 | C |
| ATOM | 10616 | OG1 | THR | B | 292 | 42.746 | 53.895 | 21.712 | 1.00 | 63.26 | O |
| ATOM | 10618 | CG2 | THR | B | 292 | 43.859 | 51.953 | 22.503 | 1.00 | 62.53 | C |
| ATOM | 10622 | C | THR | B | 292 | 42.280 | 51.766 | 24.494 | 1.00 | 57.76 | C |
| ATOM | 10623 | O | THR | B | 292 | 43.004 | 51.517 | 25.393 | 1.00 | 57.85 | O |
| ATOM | 10624 | N | GLN | B | 293 | 41.535 | 50.819 | 23.968 | 1.00 | 61.18 | N |
| ATOM | 10626 | CA | GLN | B | 293 | 41.737 | 49.429 | 24.428 | 1.00 | 66.82 | C |
| ATOM | 10628 | CB | GLN | B | 293 | 43.237 | 49.258 | 24.806 | 1.00 | 71.42 | C |
| ATOM | 10631 | CG | GLN | B | 293 | 44.001 | 48.005 | 24.291 | 1.00 | 73.25 | C |
| ATOM | 10634 | CD | GLN | B | 293 | 45.538 | 48.253 | 24.118 | 1.00 | 76.24 | C |
| ATOM | 10635 | OE1 | GLN | B | 293 | 46.178 | 47.731 | 23.180 | 1.00 | 73.24 | O |
| ATOM | 10636 | NE2 | GLN | B | 293 | 46.111 | 49.048 | 25.015 | 1.00 | 73.09 | N |
| ATOM | 10639 | C | GLN | B | 293 | 40.883 | 49.060 | 25.657 | 1.00 | 66.33 | C |
| ATOM | 10640 | O | GLN | B | 293 | 40.602 | 47.896 | 25.915 | 1.00 | 69.45 | O |
| ATOM | 10641 | N | GLN | B | 294 | 40.467 | 50.082 | 26.386 | 1.00 | 64.46 | N |
| ATOM | 10643 | CA | GLN | B | 294 | 39.810 | 50.008 | 27.690 | 1.00 | 57.83 | C |
| ATOM | 10645 | CB | GLN | B | 294 | 40.597 | 51.054 | 28.485 | 1.00 | 58.59 | C |
| ATOM | 10648 | CG | GLN | B | 294 | 40.167 | 51.551 | 29.826 | 1.00 | 57.32 | C |
| ATOM | 10651 | CD | GLN | B | 294 | 41.418 | 51.820 | 30.645 | 1.00 | 62.05 | C |
| ATOM | 10652 | OE1 | GLN | B | 294 | 41.355 | 52.330 | 31.755 | 1.00 | 72.12 | O |
| ATOM | 10653 | NE2 | GLN | B | 294 | 42.570 | 51.468 | 30.084 | 1.00 | 60.53 | N |
| ATOM | 10656 | C | GLN | B | 294 | 38.287 | 50.320 | 27.655 | 1.00 | 57.84 | C |
| ATOM | 10657 | O | GLN | B | 294 | 37.830 | 51.216 | 26.906 | 1.00 | 56.18 | O |
| ATOM | 10658 | N | SER | B | 295 | 37.490 | 49.580 | 28.443 | 1.00 | 54.23 | N |
| ATOM | 10660 | CA | SER | B | 295 | 36.050 | 49.896 | 28.573 | 1.00 | 53.45 | C |
| ATOM | 10662 | CB | SER | B | 295 | 35.178 | 49.030 | 27.672 | 1.00 | 52.98 | C |
| ATOM | 10665 | OG | SER | B | 295 | 35.006 | 47.743 | 28.227 | 1.00 | 55.81 | O |
| ATOM | 10667 | C | SER | B | 295 | 35.503 | 49.796 | 29.978 | 1.00 | 49.98 | C |
| ATOM | 10668 | O | SER | B | 295 | 36.119 | 49.194 | 30.832 | 1.00 | 47.16 | O |
| ATOM | 10669 | N | PHE | B | 296 | 34.344 | 50.406 | 30.220 | 1.00 | 48.76 | N |
| ATOM | 10671 | CA | PHE | B | 296 | 33.707 | 50.270 | 31.525 | 1.00 | 46.25 | C |
| ATOM | 10673 | CB | PHE | B | 296 | 33.853 | 51.469 | 32.349 | 1.00 | 45.49 | C |
| ATOM | 10676 | CG | PHE | B | 296 | 32.902 | 52.449 | 32.027 | 1.00 | 44.22 | C |
| ATOM | 10677 | CD1 | PHE | B | 296 | 31.723 | 52.556 | 32.738 | 1.00 | 47.00 | C |
| ATOM | 10679 | CE1 | PHE | B | 296 | 30.808 | 53.551 | 32.396 | 1.00 | 39.26 | C |
| ATOM | 10681 | CZ | PHE | B | 296 | 31.089 | 54.394 | 31.341 | 1.00 | 40.65 | C |
| ATOM | 10683 | CE2 | PHE | B | 296 | 32.268 | 54.266 | 30.638 | 1.00 | 37.93 | C |
| ATOM | 10685 | CD2 | PHE | B | 296 | 33.159 | 53.302 | 30.984 | 1.00 | 39.04 | C |
| ATOM | 10687 | C | PHE | B | 296 | 32.232 | 49.871 | 31.423 | 1.00 | 48.20 | C |
| ATOM | 10688 | O | PHE | B | 296 | 31.778 | 49.499 | 30.341 | 1.00 | 54.42 | O |
| ATOM | 10689 | N | ARG | B | 297 | 31.501 | 49.849 | 32.544 | 1.00 | 45.88 | N |
| ATOM | 10691 | CA | ARG | B | 297 | 30.130 | 49.426 | 32.526 | 1.00 | 43.75 | C |
| ATOM | 10693 | CB | ARG | B | 297 | 30.058 | 47.928 | 32.696 | 1.00 | 46.61 | C |
| ATOM | 10696 | CG | ARG | B | 297 | 30.812 | 47.407 | 33.875 | 1.00 | 52.65 | C |
| ATOM | 10699 | CD | ARG | B | 297 | 30.556 | 45.919 | 34.138 | 1.00 | 52.49 | C |
| ATOM | 10702 | NE | ARG | B | 297 | 30.710 | 45.525 | 35.546 | 1.00 | 51.81 | N |
| ATOM | 10704 | CZ | ARG | B | 297 | 31.212 | 44.358 | 35.939 | 1.00 | 48.75 | C |
| ATOM | 10705 | NH1 | ARG | B | 297 | 31.614 | 43.487 | 35.042 | 1.00 | 53.62 | N |
| ATOM | 10708 | NH2 | ARG | B | 297 | 31.317 | 44.050 | 37.222 | 1.00 | 43.21 | N |
| ATOM | 10711 | C | ARG | B | 297 | 29.314 | 50.074 | 33.615 | 1.00 | 47.03 | C |
| ATOM | 10712 | O | ARG | B | 297 | 29.852 | 50.469 | 34.676 | 1.00 | 48.02 | O |
| ATOM | 10713 | N | ILE | B | 298 | 28.010 | 50.187 | 33.294 | 1.00 | 45.30 | N |
| ATOM | 10715 | CA | ILE | B | 298 | 26.937 | 50.674 | 34.150 | 1.00 | 44.58 | C |
| ATOM | 10717 | CB | ILE | B | 298 | 26.152 | 51.846 | 33.497 | 1.00 | 42.21 | C |
| ATOM | 10719 | CG1 | ILE | B | 298 | 25.434 | 51.391 | 32.217 | 1.00 | 40.03 | C |
| ATOM | 10722 | CD1 | ILE | B | 298 | 24.548 | 52.508 | 31.592 | 1.00 | 39.09 | C |
| ATOM | 10726 | CG2 | ILE | B | 298 | 27.077 | 52.977 | 33.228 | 1.00 | 43.78 | C |
| ATOM | 10730 | C | ILE | B | 298 | 25.987 | 49.471 | 34.284 | 1.00 | 45.83 | C |
| ATOM | 10731 | O | ILE | B | 298 | 25.735 | 48.749 | 33.325 | 1.00 | 38.32 | O |
| ATOM | 10732 | N | THR | B | 299 | 25.443 | 49.270 | 35.467 | 1.00 | 46.26 | N |
| ATOM | 10734 | CA | THR | B | 299 | 24.696 | 48.072 | 35.721 | 1.00 | 47.54 | C |
| ATOM | 10736 | CB | THR | B | 299 | 25.575 | 47.181 | 36.607 | 1.00 | 52.72 | C |
| ATOM | 10738 | OG1 | THR | B | 299 | 26.587 | 46.537 | 35.810 | 1.00 | 52.36 | O |
| ATOM | 10740 | CG2 | THR | B | 299 | 24.789 | 46.025 | 37.227 | 1.00 | 53.54 | C |
| ATOM | 10744 | C | THR | B | 299 | 23.441 | 48.459 | 36.439 | 1.00 | 47.53 | C |
| ATOM | 10745 | O | THR | B | 299 | 23.554 | 49.107 | 37.478 | 1.00 | 45.39 | O |
| ATOM | 10746 | N | ILE | B | 300 | 22.265 | 48.087 | 35.887 | 1.00 | 47.99 | N |
| ATOM | 10748 | CA | ILE | B | 300 | 20.952 | 48.367 | 36.518 | 1.00 | 46.84 | C |
| ATOM | 10750 | CB | ILE | B | 300 | 19.924 | 49.104 | 35.627 | 1.00 | 46.77 | C |
| ATOM | 10752 | CG1 | ILE | B | 300 | 19.842 | 48.506 | 34.244 | 1.00 | 45.61 | C |
| ATOM | 10755 | CD1 | ILE | B | 300 | 18.475 | 48.734 | 33.633 | 1.00 | 46.44 | C |
| ATOM | 10759 | CG2 | ILE | B | 300 | 20.194 | 50.538 | 35.586 | 1.00 | 52.09 | C |
| ATOM | 10763 | C | ILE | B | 300 | 20.186 | 47.167 | 36.971 | 1.00 | 44.73 | C |
| ATOM | 10764 | O | ILE | B | 300 | 20.413 | 46.046 | 36.543 | 1.00 | 43.89 | O |
| ATOM | 10765 | N | LEU | B | 301 | 19.231 | 47.509 | 37.816 | 1.00 | 46.21 | N |
| ATOM | 10767 | CA | LEU | B | 301 | 18.355 | 46.631 | 38.524 | 1.00 | 44.01 | C |
| ATOM | 10769 | CB | LEU | B | 301 | 18.303 | 47.094 | 39.973 | 1.00 | 47.01 | C |
| ATOM | 10772 | CG | LEU | B | 301 | 19.419 | 47.990 | 40.471 | 1.00 | 40.39 | C |
| ATOM | 10774 | CD1 | LEU | B | 301 | 19.093 | 48.496 | 41.828 | 1.00 | 40.91 | C |
| ATOM | 10778 | CD2 | LEU | B | 301 | 20.671 | 47.123 | 40.543 | 1.00 | 47.03 | C |
| ATOM | 10782 | C | LEU | B | 301 | 16.936 | 46.740 | 38.060 | 1.00 | 46.44 | C |
| ATOM | 10783 | O | LEU | B | 301 | 16.540 | 47.644 | 37.336 | 1.00 | 43.22 | O |
| ATOM | 10784 | N | PRO | B | 302 | 16.136 | 45.819 | 38.547 | 1.00 | 51.74 | N |
| ATOM | 10785 | CA | PRO | B | 302 | 14.748 | 45.738 | 38.120 | 1.00 | 47.89 | C |
| ATOM | 10787 | CB | PRO | B | 302 | 14.282 | 44.417 | 38.721 | 1.00 | 51.94 | C |
| ATOM | 10790 | CG | PRO | B | 302 | 15.105 | 44.241 | 39.916 | 1.00 | 53.36 | C |
| ATOM | 10793 | CD | PRO | B | 302 | 16.475 | 44.793 | 39.553 | 1.00 | 53.46 | C |
| ATOM | 10796 | C | PRO | B | 302 | 14.008 | 46.884 | 38.695 | 1.00 | 43.06 | C |
| ATOM | 10797 | O | PRO | B | 302 | 12.940 | 47.149 | 38.231 | 1.00 | 42.92 | O |
| ATOM | 10798 | N | GLN | B | 303 | 14.582 | 47.561 | 39.681 | 1.00 | 42.87 | N |
| ATOM | 10800 | CA | GLN | B | 303 | 13.930 | 48.707 | 40.253 | 1.00 | 42.11 | C |
| ATOM | 10802 | CB | GLN | B | 303 | 14.625 | 49.192 | 41.504 | 1.00 | 40.42 | C |
| ATOM | 10805 | CG | GLN | B | 303 | 14.037 | 48.667 | 42.758 | 1.00 | 40.53 | C |
| ATOM | 10808 | CD | GLN | B | 303 | 14.744 | 47.461 | 43.285 | 1.00 | 43.06 | C |
| ATOM | 10809 | OE1 | GLN | B | 303 | 15.546 | 46.827 | 42.579 | 1.00 | 39.84 | O |
| ATOM | 10810 | NE2 | GLN | B | 303 | 14.449 | 47.126 | 44.543 | 1.00 | 43.69 | N |
| ATOM | 10813 | C | GLN | B | 303 | 14.061 | 49.794 | 39.271 | 1.00 | 45.98 | C |
| ATOM | 10814 | O | GLN | B | 303 | 13.689 | 50.934 | 39.528 | 1.00 | 53.51 | O |
| ATOM | 10815 | N | GLN | B | 304 | 14.602 | 49.469 | 38.129 | 1.00 | 47.03 | N |
| ATOM | 10817 | CA | GLN | B | 304 | 14.815 | 50.478 | 37.174 | 1.00 | 46.42 | C |
| ATOM | 10819 | CB | GLN | B | 304 | 16.294 | 50.534 | 36.921 | 1.00 | 48.97 | C |
| ATOM | 10822 | CG | GLN | B | 304 | 16.894 | 51.900 | 37.204 | 1.00 | 45.42 | C |
| ATOM | 10825 | CD | GLN | B | 304 | 17.282 | 52.050 | 38.613 | 1.00 | 42.60 | C |
| ATOM | 10826 | OE1 | GLN | B | 304 | 16.869 | 52.990 | 39.271 | 1.00 | 55.93 | O |
| ATOM | 10827 | NE2 | GLN | B | 304 | 18.088 | 51.152 | 39.088 | 1.00 | 43.68 | N |
| ATOM | 10830 | C | GLN | B | 304 | 14.081 | 50.144 | 35.904 | 1.00 | 51.85 | C |
| ATOM | 10831 | O | GLN | B | 304 | 13.344 | 51.021 | 35.361 | 1.00 | 53.64 | O |
| ATOM | 10832 | N | TYR | B | 305 | 14.276 | 48.905 | 35.420 | 1.00 | 49.13 | N |
| ATOM | 10834 | CA | TYR | B | 305 | 13.624 | 48.452 | 34.192 | 1.00 | 48.73 | C |
| ATOM | 10836 | CB | TYR | B | 305 | 14.445 | 47.418 | 33.409 | 1.00 | 48.24 | C |
| ATOM | 10839 | CG | TYR | B | 305 | 14.696 | 46.120 | 34.075 | 1.00 | 45.68 | C |
| ATOM | 10840 | CD1 | TYR | B | 305 | 13.829 | 45.057 | 33.906 | 1.00 | 49.08 | C |
| ATOM | 10842 | CE1 | TYR | B | 305 | 14.056 | 43.872 | 34.505 | 1.00 | 51.64 | C |
| ATOM | 10844 | CZ | TYR | B | 305 | 15.186 | 43.711 | 35.301 | 1.00 | 59.15 | C |
| ATOM | 10845 | OH | TYR | B | 305 | 15.454 | 42.500 | 35.932 | 1.00 | 61.84 | O |
| ATOM | 10847 | CE2 | TYR | B | 305 | 16.063 | 44.765 | 35.467 | 1.00 | 56.26 | C |
| ATOM | 10849 | CD2 | TYR | B | 305 | 15.805 | 45.948 | 34.857 | 1.00 | 48.39 | C |
| ATOM | 10851 | C | TYR | B | 305 | 12.264 | 47.897 | 34.416 | 1.00 | 51.41 | C |
| ATOM | 10852 | O | TYR | B | 305 | 11.637 | 47.411 | 33.467 | 1.00 | 50.10 | O |
| ATOM | 10853 | N | LEU | B | 306 | 11.807 | 47.952 | 35.662 | 1.00 | 51.83 | N |
| ATOM | 10855 | CA | LEU | B | 306 | 10.481 | 47.474 | 35.996 | 1.00 | 49.68 | C |
| ATOM | 10857 | CB | LEU | B | 306 | 10.508 | 46.481 | 37.129 | 1.00 | 52.01 | C |
| ATOM | 10860 | CG | LEU | B | 306 | 10.687 | 45.019 | 36.827 | 1.00 | 51.56 | C |
| ATOM | 10862 | CD1 | LEU | B | 306 | 10.108 | 44.267 | 38.015 | 1.00 | 53.34 | C |
| ATOM | 10866 | CD2 | LEU | B | 306 | 9.924 | 44.708 | 35.605 | 1.00 | 56.13 | C |
| ATOM | 10870 | C | LEU | B | 306 | 9.884 | 48.717 | 36.499 | 1.00 | 50.94 | C |
| ATOM | 10871 | O | LEU | B | 306 | 10.282 | 49.209 | 37.547 | 1.00 | 50.28 | O |
| ATOM | 10872 | N | ARG | B | 307 | 8.908 | 49.212 | 35.764 | 1.00 | 49.70 | N |
| ATOM | 10874 | CA | ARG | B | 307 | 8.419 | 50.538 | 35.974 | 1.00 | 44.12 | C |
| ATOM | 10876 | CB | ARG | B | 307 | 8.456 | 51.259 | 34.633 | 1.00 | 38.83 | C |
| ATOM | 10879 | CG | ARG | B | 307 | 7.667 | 52.549 | 34.573 | 1.00 | 42.47 | C |
| ATOM | 10882 | CD | ARG | B | 307 | 7.695 | 53.176 | 33.188 | 1.00 | 46.67 | C |
| ATOM | 10885 | NE | ARG | B | 307 | 6.899 | 52.434 | 32.208 | 1.00 | 49.09 | N |
| ATOM | 10887 | CZ | ARG | B | 307 | 5.744 | 52.870 | 31.717 | 1.00 | 55.81 | C |
| ATOM | 10888 | NH1 | ARG | B | 307 | 5.245 | 54.035 | 32.116 | 1.00 | 54.72 | N |
| ATOM | 10891 | NH2 | ARG | B | 307 | 5.072 | 52.145 | 30.825 | 1.00 | 58.11 | N |
| ATOM | 10894 | C | ARG | B | 307 | 7.038 | 50.495 | 36.548 | 1.00 | 46.97 | C |
| ATOM | 10895 | O | ARG | B | 307 | 6.218 | 49.805 | 35.992 | 1.00 | 51.21 | O |
| ATOM | 10896 | N | PRO | B | 308 | 6.799 | 51.239 | 37.629 | 1.00 | 46.48 | N |
| ATOM | 10897 | CA | PRO | B | 308 | 5.547 | 51.289 | 38.338 | 1.00 | 50.59 | C |
| ATOM | 10899 | CB | PRO | B | 308 | 5.892 | 52.087 | 39.593 | 1.00 | 48.57 | C |
| ATOM | 10902 | CG | PRO | B | 308 | 7.261 | 52.256 | 39.619 | 1.00 | 49.01 | C |
| ATOM | 10905 | CD | PRO | B | 308 | 7.773 | 52.123 | 38.259 | 1.00 | 50.08 | C |
| ATOM | 10908 | C | PRO | B | 308 | 4.465 | 52.087 | 37.633 | 1.00 | 59.61 | C |
| ATOM | 10909 | O | PRO | B | 308 | 4.792 | 52.993 | 36.867 | 1.00 | 64.89 | O |
| ATOM | 10910 | N | VAL | B | 309 | 3.200 | 51.774 | 37.946 | 1.00 | 62.04 | N |
| ATOM | 10912 | CA | VAL | B | 309 | 2.026 | 52.332 | 37.288 | 1.00 | 64.12 | C |
| ATOM | 10914 | CB | VAL | B | 309 | 1.720 | 51.597 | 35.938 | 1.00 | 65.00 | C |
| ATOM | 10916 | CG1 | VAL | B | 309 | 2.994 | 51.101 | 35.294 | 1.00 | 63.78 | C |
| ATOM | 10920 | CG2 | VAL | B | 309 | 0.825 | 50.415 | 36.150 | 1.00 | 64.85 | C |
| ATOM | 10924 | C | VAL | B | 309 | 0.810 | 52.137 | 38.173 | 1.00 | 64.34 | C |
| ATOM | 10925 | O | VAL | B | 309 | 0.764 | 51.261 | 39.002 | 1.00 | 55.43 | O |
| ATOM | 10926 | N | GLU | B | 310 | −0.215 | 52.936 | 37.977 | 1.00 | 70.92 | N |
| ATOM | 10928 | CA | GLU | B | 310 | −1.368 | 52.762 | 38.834 | 1.00 | 74.98 | C |
| ATOM | 10930 | CB | GLU | B | 310 | −2.380 | 53.877 | 38.651 | 1.00 | 76.84 | C |
| ATOM | 10933 | CG | GLU | B | 310 | −3.286 | 54.103 | 39.862 | 1.00 | 82.90 | C |
| ATOM | 10936 | CD | GLU | B | 310 | −2.610 | 53.829 | 41.205 | 1.00 | 84.06 | C |
| ATOM | 10937 | OE1 | GLU | B | 310 | −2.619 | 52.653 | 41.631 | 1.00 | 90.22 | O |
| ATOM | 10938 | OE2 | GLU | B | 310 | −2.082 | 54.774 | 41.841 | 1.00 | 83.28 | O |
| ATOM | 10939 | C | GLU | B | 310 | −1.978 | 51.443 | 38.473 | 1.00 | 76.13 | C |
| ATOM | 10940 | O | GLU | B | 310 | −1.908 | 51.029 | 37.296 | 1.00 | 76.20 | O |
| ATOM | 10941 | N | ASP | B | 311 | −2.558 | 50.766 | 39.470 | 1.00 | 78.57 | N |
| ATOM | 10943 | CA | ASP | B | 311 | −3.306 | 49.539 | 39.182 | 1.00 | 82.17 | C |
| ATOM | 10945 | CB | ASP | B | 311 | −3.558 | 48.684 | 40.441 | 1.00 | 82.68 | C |
| ATOM | 10948 | CG | ASP | B | 311 | −3.918 | 47.211 | 40.111 | 1.00 | 83.41 | C |
| ATOM | 10949 | OD1 | ASP | B | 311 | −4.210 | 46.905 | 38.926 | 1.00 | 86.04 | O |
| ATOM | 10950 | OD2 | ASP | B | 311 | −3.931 | 46.295 | 40.976 | 1.00 | 76.70 | O |
| ATOM | 10951 | C | ASP | B | 311 | −4.643 | 49.948 | 38.570 | 1.00 | 83.78 | C |
| ATOM | 10952 | O | ASP | B | 311 | −5.174 | 51.026 | 38.867 | 1.00 | 81.54 | O |
| ATOM | 10953 | N | VAL | B | 312 | −5.172 | 49.100 | 37.707 | 1.00 | 85.51 | N |
| ATOM | 10955 | CA | VAL | B | 312 | −6.496 | 49.341 | 37.181 | 1.00 | 88.99 | C |
| ATOM | 10957 | CB | VAL | B | 312 | −7.065 | 48.064 | 36.499 | 1.00 | 88.37 | C |
| ATOM | 10959 | CG1 | VAL | B | 312 | −8.222 | 48.405 | 35.595 | 1.00 | 88.24 | C |
| ATOM | 10963 | CG2 | VAL | B | 312 | −5.979 | 47.356 | 35.697 | 1.00 | 88.52 | C |
| ATOM | 10967 | C | VAL | B | 312 | −7.361 | 49.769 | 38.383 | 1.00 | 92.51 | C |
| ATOM | 10968 | O | VAL | B | 312 | −7.811 | 50.910 | 38.482 | 1.00 | 94.93 | O |
| ATOM | 10969 | N | ALA | B | 313 | −7.563 | 48.866 | 39.331 | 1.00 | 96.03 | N |
| ATOM | 10971 | CA | ALA | B | 313 | −8.432 | 49.151 | 40.470 | 1.00 | 97.31 | C |
| ATOM | 10973 | CB | ALA | B | 313 | −8.438 | 47.916 | 41.424 | 1.00 | 95.55 | C |
| ATOM | 10977 | C | ALA | B | 313 | −8.055 | 50.404 | 41.281 | 1.00 | 98.18 | C |
| ATOM | 10978 | O | ALA | B | 313 | −8.868 | 50.852 | 42.088 | 1.00 | 102.35 | O |
| ATOM | 10979 | N | THR | B | 314 | −6.864 | 50.971 | 41.033 | 1.00 | 97.48 | N |
| ATOM | 10981 | CA | THR | B | 314 | −6.102 | 51.818 | 42.013 | 1.00 | 96.57 | C |
| ATOM | 10983 | CB | THR | B | 314 | −6.505 | 53.356 | 42.037 | 1.00 | 95.77 | C |
| ATOM | 10985 | OG1 | THR | B | 314 | −7.547 | 53.635 | 42.985 | 1.00 | 95.94 | O |
| ATOM | 10987 | CG2 | THR | B | 314 | −7.040 | 53.811 | 40.662 | 1.00 | 95.13 | C |
| ATOM | 10991 | C | THR | B | 314 | −6.028 | 51.095 | 43.417 | 1.00 | 94.99 | C |
| ATOM | 10992 | O | THR | B | 314 | −6.148 | 51.703 | 44.505 | 1.00 | 89.26 | O |
| ATOM | 10993 | N | SER | B | 315 | −5.810 | 49.767 | 43.307 | 1.00 | 94.79 | N |
| ATOM | 10995 | CA | SER | B | 315 | −5.731 | 48.796 | 44.424 | 1.00 | 93.42 | C |
| ATOM | 10997 | CB | SER | B | 315 | −5.712 | 47.335 | 43.911 | 1.00 | 93.27 | C |
| ATOM | 11000 | OG | SER | B | 315 | −4.568 | 47.043 | 43.118 | 1.00 | 85.86 | O |
| ATOM | 11002 | C | SER | B | 315 | −4.516 | 49.033 | 45.306 | 1.00 | 93.27 | C |
| ATOM | 11003 | O | SER | B | 315 | −3.681 | 49.893 | 44.995 | 1.00 | 94.12 | O |
| ATOM | 11004 | N | GLN | B | 316 | −4.410 | 48.279 | 46.404 | 1.00 | 91.57 | N |
| ATOM | 11006 | CA | GLN | B | 316 | −3.318 | 48.533 | 47.354 | 1.00 | 91.29 | C |
| ATOM | 11008 | CB | GLN | B | 316 | −3.756 | 48.294 | 48.839 | 1.00 | 92.38 | C |
| ATOM | 11011 | CG | GLN | B | 316 | −4.329 | 49.622 | 49.500 | 1.00 | 93.01 | C |
| ATOM | 11014 | CD | GLN | B | 316 | −4.619 | 49.576 | 51.022 | 1.00 | 91.31 | C |
| ATOM | 11015 | OE1 | GLN | B | 316 | −4.691 | 48.505 | 51.626 | 1.00 | 90.86 | O |
| ATOM | 11016 | NE2 | GLN | B | 316 | −4.791 | 50.762 | 51.629 | 1.00 | 89.33 | N |
| ATOM | 11019 | C | GLN | B | 316 | −2.010 | 47.823 | 46.923 | 1.00 | 85.48 | C |
| ATOM | 11020 | O | GLN | B | 316 | −0.926 | 48.158 | 47.397 | 1.00 | 84.70 | O |
| ATOM | 11021 | N | ASP | B | 317 | −2.129 | 46.872 | 46.001 | 1.00 | 78.17 | N |
| ATOM | 11023 | CA | ASP | B | 317 | −0.980 | 46.180 | 45.434 | 1.00 | 76.19 | C |
| ATOM | 11025 | CB | ASP | B | 317 | −1.492 | 45.121 | 44.472 | 1.00 | 73.46 | C |
| ATOM | 11028 | CG | ASP | B | 317 | −2.262 | 44.078 | 45.184 | 1.00 | 72.17 | C |
| ATOM | 11029 | OD1 | ASP | B | 317 | −2.407 | 44.252 | 46.424 | 1.00 | 70.34 | O |
| ATOM | 11030 | OD2 | ASP | B | 317 | −2.743 | 43.073 | 44.620 | 1.00 | 70.32 | O |
| ATOM | 11031 | C | ASP | B | 317 | 0.034 | 47.099 | 44.728 | 1.00 | 76.88 | C |
| ATOM | 11032 | O | ASP | B | 317 | −0.247 | 48.268 | 44.447 | 1.00 | 82.57 | O |
| ATOM | 11033 | N | ASP | B | 318 | 1.216 | 46.558 | 44.448 | 1.00 | 74.00 | N |
| ATOM | 11035 | CA | ASP | B | 318 | 2.304 | 47.312 | 43.838 | 1.00 | 68.80 | C |
| ATOM | 11037 | CB | ASP | B | 318 | 3.577 | 47.191 | 44.686 | 1.00 | 70.82 | C |
| ATOM | 11040 | CG | ASP | B | 318 | 3.534 | 48.035 | 45.962 | 1.00 | 71.07 | C |
| ATOM | 11041 | OD1 | ASP | B | 318 | 4.185 | 47.662 | 46.968 | 1.00 | 70.80 | O |
| ATOM | 11042 | OD2 | ASP | B | 318 | 2.884 | 49.087 | 46.052 | 1.00 | 73.30 | O |
| ATOM | 11043 | C | ASP | B | 318 | 2.511 | 46.719 | 42.455 | 1.00 | 65.62 | C |
| ATOM | 11044 | O | ASP | B | 318 | 3.019 | 45.608 | 42.319 | 1.00 | 60.87 | O |
| ATOM | 11045 | N | CYS | B | 319 | 2.126 | 47.456 | 41.419 | 1.00 | 61.57 | N |
| ATOM | 11047 | CA | CYS | B | 319 | 2.133 | 46.872 | 40.093 | 1.00 | 57.97 | C |
| ATOM | 11049 | CB | CYS | B | 319 | 0.736 | 46.962 | 39.463 | 1.00 | 61.08 | C |
| ATOM | 11052 | SG | CYS | B | 319 | −0.613 | 46.137 | 40.403 | 1.00 | 68.12 | S |
| ATOM | 11053 | C | CYS | B | 319 | 3.220 | 47.524 | 39.269 | 1.00 | 52.64 | C |
| ATOM | 11054 | O | CYS | B | 319 | 3.745 | 48.540 | 39.662 | 1.00 | 52.88 | O |
| ATOM | 11055 | N | TYR | B | 320 | 3.560 | 46.945 | 38.130 | 1.00 | 47.04 | N |
| ATOM | 11057 | CA | TYR | B | 320 | 4.728 | 47.385 | 37.415 | 1.00 | 50.79 | C |
| ATOM | 11059 | CB | TYR | B | 320 | 6.042 | 46.773 | 38.010 | 1.00 | 51.87 | C |
| ATOM | 11062 | CG | TYR | B | 320 | 6.341 | 47.116 | 39.469 | 1.00 | 44.28 | C |
| ATOM | 11063 | CD1 | TYR | B | 320 | 5.861 | 46.331 | 40.512 | 1.00 | 51.75 | C |
| ATOM | 11065 | CE1 | TYR | B | 320 | 6.125 | 46.637 | 41.826 | 1.00 | 48.81 | C |
| ATOM | 11067 | CZ | TYR | B | 320 | 6.873 | 47.737 | 42.090 | 1.00 | 51.67 | C |
| ATOM | 11068 | OH | TYR | B | 320 | 7.161 | 48.090 | 43.379 | 1.00 | 63.11 | O |
| ATOM | 11070 | CE2 | TYR | B | 320 | 7.351 | 48.512 | 41.073 | 1.00 | 46.32 | C |
| ATOM | 11072 | CD2 | TYR | B | 320 | 7.082 | 48.188 | 39.779 | 1.00 | 35.82 | C |
| ATOM | 11074 | C | TYR | B | 320 | 4.626 | 46.882 | 36.016 | 1.00 | 52.12 | C |
| ATOM | 11075 | O | TYR | B | 320 | 4.082 | 45.809 | 35.792 | 1.00 | 56.07 | O |
| ATOM | 11076 | N | LYS | B | 321 | 5.164 | 47.667 | 35.085 | 1.00 | 53.09 | N |
| ATOM | 11078 | CA | LYS | B | 321 | 5.216 | 47.294 | 33.708 | 1.00 | 51.68 | C |
| ATOM | 11080 | CB | LYS | B | 321 | 4.684 | 48.430 | 32.832 | 1.00 | 57.10 | C |
| ATOM | 11083 | CG | LYS | B | 321 | 3.102 | 48.538 | 32.635 | 1.00 | 61.86 | C |
| ATOM | 11086 | CD | LYS | B | 321 | 2.727 | 49.287 | 31.269 | 1.00 | 67.16 | C |
| ATOM | 11089 | CE | LYS | B | 321 | 1.532 | 50.345 | 31.323 | 1.00 | 70.93 | C |
| ATOM | 11092 | NZ | LYS | B | 321 | 1.847 | 51.846 | 31.459 | 1.00 | 71.64 | N |
| ATOM | 11096 | C | LYS | B | 321 | 6.681 | 47.049 | 33.421 | 1.00 | 49.27 | C |
| ATOM | 11097 | O | LYS | B | 321 | 7.544 | 47.694 | 33.987 | 1.00 | 54.13 | O |
| ATOM | 11098 | N | PHE | B | 322 | 6.961 | 46.083 | 32.568 | 1.00 | 45.37 | N |
| ATOM | 11100 | CA | PHE | B | 322 | 8.291 | 45.825 | 32.087 | 1.00 | 45.16 | C |
| ATOM | 11102 | CB | PHE | B | 322 | 8.218 | 44.478 | 31.390 | 1.00 | 41.79 | C |
| ATOM | 11105 | CG | PHE | B | 322 | 9.528 | 43.929 | 30.875 | 1.00 | 42.80 | C |
| ATOM | 11106 | CD1 | PHE | B | 322 | 10.627 | 43.747 | 31.697 | 1.00 | 41.54 | C |
| ATOM | 11108 | CE1 | PHE | B | 322 | 11.812 | 43.202 | 31.188 | 1.00 | 46.06 | C |
| ATOM | 11110 | CZ | PHE | B | 322 | 11.895 | 42.839 | 29.861 | 1.00 | 44.89 | C |
| ATOM | 11112 | CE2 | PHE | B | 322 | 10.803 | 43.031 | 29.042 | 1.00 | 34.54 | C |
| ATOM | 11114 | CD2 | PHE | B | 322 | 9.633 | 43.549 | 29.547 | 1.00 | 37.25 | C |
| ATOM | 11116 | C | PHE | B | 322 | 8.606 | 46.957 | 31.087 | 1.00 | 48.75 | C |
| ATOM | 11117 | O | PHE | B | 322 | 7.894 | 47.126 | 30.099 | 1.00 | 56.32 | O |
| ATOM | 11118 | N | ALA | B | 323 | 9.651 | 47.736 | 31.346 | 1.00 | 49.68 | N |
| ATOM | 11120 | CA | ALA | B | 323 | 10.098 | 48.768 | 30.413 | 1.00 | 46.88 | C |
| ATOM | 11122 | CB | ALA | B | 323 | 10.314 | 50.060 | 31.172 | 1.00 | 46.63 | C |
| ATOM | 11126 | C | ALA | B | 323 | 11.377 | 48.461 | 29.609 | 1.00 | 45.38 | C |
| ATOM | 11127 | O | ALA | B | 323 | 12.397 | 49.107 | 29.792 | 1.00 | 55.92 | O |
| ATOM | 11128 | N | ILE | B | 324 | 11.332 | 47.502 | 28.713 | 1.00 | 48.17 | N |
| ATOM | 11130 | CA | ILE | B | 324 | 12.466 | 47.206 | 27.845 | 1.00 | 46.85 | C |
| ATOM | 11132 | CB | ILE | B | 324 | 13.345 | 46.064 | 28.445 | 1.00 | 44.62 | C |
| ATOM | 11134 | CG1 | ILE | B | 324 | 14.171 | 46.476 | 29.668 | 1.00 | 51.60 | C |
| ATOM | 11137 | CD1 | ILE | B | 324 | 15.228 | 45.430 | 30.124 | 1.00 | 57.66 | C |
| ATOM | 11141 | CG2 | ILE | B | 324 | 14.315 | 45.545 | 27.437 | 1.00 | 40.76 | C |
| ATOM | 11145 | C | ILE | B | 324 | 11.754 | 46.719 | 26.575 | 1.00 | 49.80 | C |
| ATOM | 11146 | O | ILE | B | 324 | 11.146 | 45.664 | 26.644 | 1.00 | 50.08 | O |
| ATOM | 11147 | N | SER | B | 325 | 11.794 | 47.464 | 25.446 | 1.00 | 50.30 | N |
| ATOM | 11149 | CA | SER | B | 325 | 11.092 | 47.067 | 24.174 | 1.00 | 50.46 | C |
| ATOM | 11151 | CB | SER | B | 325 | 10.034 | 48.087 | 23.799 | 1.00 | 49.93 | C |
| ATOM | 11154 | OG | SER | B | 325 | 9.963 | 49.115 | 24.782 | 1.00 | 59.75 | O |
| ATOM | 11156 | C | SER | B | 325 | 11.954 | 46.918 | 22.928 | 1.00 | 50.03 | C |
| ATOM | 11157 | O | SER | B | 325 | 13.119 | 47.211 | 22.936 | 1.00 | 53.12 | O |
| ATOM | 11158 | N | GLN | B | 326 | 11.382 | 46.465 | 21.828 | 1.00 | 56.80 | N |
| ATOM | 11160 | CA | GLN | B | 326 | 12.193 | 46.354 | 20.631 | 1.00 | 59.61 | C |
| ATOM | 11162 | CB | GLN | B | 326 | 11.717 | 45.312 | 19.617 | 1.00 | 60.94 | C |
| ATOM | 11165 | CG | GLN | B | 326 | 10.391 | 44.672 | 19.856 | 1.00 | 66.47 | C |
| ATOM | 11168 | CD | GLN | B | 326 | 10.292 | 43.338 | 19.116 | 1.00 | 74.18 | C |
| ATOM | 11169 | OE1 | GLN | B | 326 | 10.878 | 43.178 | 18.023 | 1.00 | 71.53 | O |
| ATOM | 11170 | NE2 | GLN | B | 326 | 9.553 | 42.374 | 19.707 | 1.00 | 75.43 | N |
| ATOM | 11173 | C | GLN | B | 326 | 12.238 | 47.707 | 19.965 | 1.00 | 60.48 | C |
| ATOM | 11174 | O | GLN | B | 326 | 11.435 | 48.586 | 20.264 | 1.00 | 57.03 | O |
| ATOM | 11175 | N | SER | B | 327 | 13.198 | 47.844 | 19.064 | 1.00 | 57.25 | N |
| ATOM | 11177 | CA | SER | B | 327 | 13.413 | 49.051 | 18.331 | 1.00 | 55.49 | C |
| ATOM | 11179 | CB | SER | B | 327 | 14.670 | 49.752 | 18.824 | 1.00 | 53.74 | C |
| ATOM | 11182 | OG | SER | B | 327 | 15.511 | 50.178 | 17.714 | 1.00 | 49.78 | O |
| ATOM | 11184 | C | SER | B | 327 | 13.654 | 48.642 | 16.914 | 1.00 | 58.77 | C |
| ATOM | 11185 | O | SER | B | 327 | 14.087 | 47.518 | 16.656 | 1.00 | 59.83 | O |
| ATOM | 11186 | N | SER | B | 328 | 13.379 | 49.541 | 15.984 | 1.00 | 59.17 | N |
| ATOM | 11188 | CA | SER | B | 328 | 13.786 | 49.287 | 14.625 | 1.00 | 60.92 | C |
| ATOM | 11190 | CB | SER | B | 328 | 12.606 | 49.365 | 13.631 | 1.00 | 63.09 | C |
| ATOM | 11193 | OG | SER | B | 328 | 11.492 | 50.158 | 14.067 | 1.00 | 65.22 | O |
| ATOM | 11195 | C | SER | B | 328 | 14.911 | 50.259 | 14.300 | 1.00 | 60.93 | C |
| ATOM | 11196 | O | SER | B | 328 | 15.657 | 50.066 | 13.337 | 1.00 | 64.26 | O |
| ATOM | 11197 | N | THR | B | 329 | 15.076 | 51.287 | 15.131 | 1.00 | 61.31 | N |
| ATOM | 11199 | CA | THR | B | 329 | 16.051 | 52.372 | 14.845 | 1.00 | 59.73 | C |
| ATOM | 11201 | CB | THR | B | 329 | 15.359 | 53.753 | 14.900 | 1.00 | 60.54 | C |
| ATOM | 11203 | OG1 | THR | B | 329 | 14.584 | 53.860 | 16.105 | 1.00 | 58.14 | O |
| ATOM | 11205 | CG2 | THR | B | 329 | 14.332 | 53.922 | 13.743 | 1.00 | 56.98 | C |
| ATOM | 11209 | C | THR | B | 329 | 17.278 | 52.431 | 15.749 | 1.00 | 58.54 | C |
| ATOM | 11210 | O | THR | B | 329 | 17.933 | 53.459 | 15.778 | 1.00 | 61.65 | O |
| ATOM | 11211 | N | GLY | B | 330 | 17.567 | 51.325 | 16.460 | 1.00 | 58.25 | N |
| ATOM | 11213 | CA | GLY | B | 330 | 18.695 | 51.165 | 17.351 | 1.00 | 50.47 | C |
| ATOM | 11216 | C | GLY | B | 330 | 18.313 | 51.180 | 18.850 | 1.00 | 52.50 | C |
| ATOM | 11217 | O | GLY | B | 330 | 17.128 | 51.312 | 19.252 | 1.00 | 56.44 | O |
| ATOM | 11218 | N | THR | B | 331 | 19.343 | 51.046 | 19.686 | 1.00 | 45.78 | N |
| ATOM | 11220 | CA | THR | B | 331 | 19.241 | 51.078 | 21.119 | 1.00 | 41.85 | C |
| ATOM | 11222 | CB | THR | B | 331 | 20.630 | 50.637 | 21.677 | 1.00 | 41.95 | C |
| ATOM | 11224 | OG1 | THR | B | 331 | 20.726 | 49.210 | 21.674 | 1.00 | 47.12 | O |
| ATOM | 11226 | CG2 | THR | B | 331 | 20.797 | 50.988 | 23.053 | 1.00 | 37.45 | C |
| ATOM | 11230 | C | THR | B | 331 | 18.966 | 52.502 | 21.548 | 1.00 | 41.86 | C |
| ATOM | 11231 | O | THR | B | 331 | 19.675 | 53.422 | 21.115 | 1.00 | 42.95 | O |
| ATOM | 11232 | N | VAL | B | 332 | 17.961 | 52.691 | 22.401 | 1.00 | 37.91 | N |
| ATOM | 11234 | CA | VAL | B | 332 | 17.747 | 53.961 | 23.049 | 1.00 | 36.68 | C |
| ATOM | 11236 | CB | VAL | B | 332 | 16.372 | 54.558 | 22.862 | 1.00 | 38.12 | C |
| ATOM | 11238 | CG1 | VAL | B | 332 | 16.453 | 56.041 | 22.986 | 1.00 | 37.57 | C |
| ATOM | 11242 | CG2 | VAL | B | 332 | 15.785 | 54.214 | 21.527 | 1.00 | 42.36 | C |
| ATOM | 11246 | C | VAL | B | 332 | 17.891 | 53.693 | 24.528 | 1.00 | 43.79 | C |
| ATOM | 11247 | O | VAL | B | 332 | 17.094 | 52.953 | 25.121 | 1.00 | 44.24 | O |
| ATOM | 11248 | N | MET | B | 333 | 18.931 | 54.302 | 25.104 | 1.00 | 46.87 | N |
| ATOM | 11250 | CA | MET | B | 333 | 19.231 | 54.228 | 26.508 | 1.00 | 43.89 | C |
| ATOM | 11252 | CB | MET | B | 333 | 20.720 | 54.492 | 26.737 | 1.00 | 43.44 | C |
| ATOM | 11255 | CG | MET | B | 333 | 21.665 | 53.638 | 25.852 | 1.00 | 44.02 | C |
| ATOM | 11258 | SD | MET | B | 333 | 23.458 | 53.802 | 26.313 | 1.00 | 55.34 | S |
| ATOM | 11259 | CE | MET | B | 333 | 23.460 | 53.010 | 27.911 | 1.00 | 55.88 | C |
| ATOM | 11263 | C | MET | B | 333 | 18.368 | 55.332 | 27.098 | 1.00 | 48.39 | C |
| ATOM | 11264 | O | MET | B | 333 | 18.802 | 56.474 | 27.222 | 1.00 | 53.18 | O |
| ATOM | 11265 | N | GLY | B | 334 | 17.134 | 54.979 | 27.451 | 1.00 | 50.35 | N |
| ATOM | 11267 | CA | GLY | B | 334 | 16.145 | 55.935 | 27.929 | 1.00 | 49.72 | C |
| ATOM | 11270 | C | GLY | B | 334 | 16.128 | 56.215 | 29.401 | 1.00 | 48.78 | C |
| ATOM | 11271 | O | GLY | B | 334 | 17.099 | 55.961 | 30.105 | 1.00 | 58.24 | O |
| ATOM | 11272 | N | ALA | B | 335 | 14.985 | 56.698 | 29.865 | 1.00 | 48.59 | N |
| ATOM | 11274 | CA | ALA | B | 335 | 14.888 | 57.271 | 31.193 | 1.00 | 50.40 | C |
| ATOM | 11276 | CB | ALA | B | 335 | 13.612 | 58.070 | 31.297 | 1.00 | 48.07 | C |
| ATOM | 11280 | C | ALA | B | 335 | 15.002 | 56.266 | 32.338 | 1.00 | 53.66 | C |
| ATOM | 11281 | O | ALA | B | 335 | 15.461 | 56.596 | 33.415 | 1.00 | 58.54 | O |
| ATOM | 11282 | N | VAL | B | 336 | 14.586 | 55.035 | 32.135 | 1.00 | 53.31 | N |
| ATOM | 11284 | CA | VAL | B | 336 | 14.750 | 54.108 | 33.224 | 1.00 | 55.63 | C |
| ATOM | 11286 | CB | VAL | B | 336 | 13.937 | 52.808 | 33.058 | 1.00 | 58.93 | C |
| ATOM | 11288 | CG1 | VAL | B | 336 | 12.409 | 53.162 | 33.026 | 1.00 | 56.89 | C |
| ATOM | 11292 | CG2 | VAL | B | 336 | 14.432 | 51.979 | 31.840 | 1.00 | 58.15 | C |
| ATOM | 11296 | C | VAL | B | 336 | 16.202 | 53.785 | 33.317 | 1.00 | 53.53 | C |
| ATOM | 11297 | O | VAL | B | 336 | 16.666 | 53.274 | 34.321 | 1.00 | 57.81 | O |
| ATOM | 11298 | N | ILE | B | 337 | 16.946 | 54.059 | 32.267 | 1.00 | 50.75 | N |
| ATOM | 11300 | CA | ILE | B | 337 | 18.367 | 53.801 | 32.370 | 1.00 | 46.62 | C |
| ATOM | 11302 | CB | ILE | B | 337 | 19.056 | 53.626 | 30.992 | 1.00 | 43.03 | C |
| ATOM | 11304 | CG1 | ILE | B | 337 | 18.218 | 52.737 | 30.091 | 1.00 | 45.86 | C |
| ATOM | 11307 | CD1 | ILE | B | 337 | 17.973 | 51.333 | 30.721 | 1.00 | 45.07 | C |
| ATOM | 11311 | CG2 | ILE | B | 337 | 20.355 | 52.901 | 31.105 | 1.00 | 44.92 | C |
| ATOM | 11315 | C | ILE | B | 337 | 18.785 | 55.035 | 33.102 | 1.00 | 48.16 | C |
| ATOM | 11316 | O | ILE | B | 337 | 19.334 | 54.925 | 34.164 | 1.00 | 51.02 | O |
| ATOM | 11317 | N | MET | B | 338 | 18.460 | 56.219 | 32.583 | 1.00 | 45.36 | N |
| ATOM | 11319 | CA | MET | B | 338 | 18.968 | 57.443 | 33.185 | 1.00 | 44.08 | C |
| ATOM | 11321 | CB | MET | B | 338 | 18.460 | 58.679 | 32.452 | 1.00 | 47.75 | C |
| ATOM | 11324 | CG | MET | B | 338 | 18.833 | 58.740 | 31.003 | 1.00 | 48.46 | C |
| ATOM | 11327 | SD | MET | B | 338 | 18.465 | 60.332 | 30.349 | 1.00 | 59.21 | S |
| ATOM | 11328 | CE | MET | B | 338 | 19.867 | 61.227 | 30.953 | 1.00 | 56.77 | C |
| ATOM | 11332 | C | MET | B | 338 | 18.647 | 57.631 | 34.647 | 1.00 | 42.50 | C |
| ATOM | 11333 | O | MET | B | 338 | 19.328 | 58.383 | 35.313 | 1.00 | 49.24 | O |
| ATOM | 11334 | N | GLU | B | 339 | 17.629 | 56.964 | 35.148 | 1.00 | 42.14 | N |
| ATOM | 11336 | CA | GLU | B | 339 | 17.161 | 57.177 | 36.515 | 1.00 | 46.39 | C |
| ATOM | 11338 | CB | GLU | B | 339 | 15.730 | 56.656 | 36.689 | 1.00 | 50.35 | C |
| ATOM | 11341 | CG | GLU | B | 339 | 14.662 | 57.340 | 35.854 | 1.00 | 53.30 | C |
| ATOM | 11344 | CD | GLU | B | 339 | 13.241 | 57.029 | 36.346 | 1.00 | 63.08 | C |
| ATOM | 11345 | OE1 | GLU | B | 339 | 12.502 | 57.979 | 36.701 | 1.00 | 64.18 | O |
| ATOM | 11346 | OE2 | GLU | B | 339 | 12.851 | 55.834 | 36.381 | 1.00 | 65.96 | O |
| ATOM | 11347 | C | GLU | B | 339 | 18.056 | 56.512 | 37.539 | 1.00 | 48.49 | C |
| ATOM | 11348 | O | GLU | B | 339 | 17.999 | 56.842 | 38.714 | 1.00 | 52.78 | O |
| ATOM | 11349 | N | GLY | B | 340 | 18.871 | 55.565 | 37.093 | 1.00 | 47.81 | N |
| ATOM | 11351 | CA | GLY | B | 340 | 19.898 | 54.993 | 37.941 | 1.00 | 48.44 | C |
| ATOM | 11354 | C | GLY | B | 340 | 21.164 | 55.846 | 38.027 | 1.00 | 49.58 | C |
| ATOM | 11355 | O | GLY | B | 340 | 21.952 | 55.773 | 38.986 | 1.00 | 46.13 | O |
| ATOM | 11356 | N | PHE | B | 341 | 21.369 | 56.684 | 37.027 | 1.00 | 49.56 | N |
| ATOM | 11358 | CA | PHE | B | 341 | 22.598 | 57.449 | 36.967 | 1.00 | 48.86 | C |
| ATOM | 11360 | CB | PHE | B | 341 | 23.349 | 56.930 | 35.754 | 1.00 | 48.52 | C |
| ATOM | 11363 | CG | PHE | B | 341 | 23.480 | 55.442 | 35.737 | 1.00 | 47.00 | C |
| ATOM | 11364 | CD1 | PHE | B | 341 | 22.679 | 54.674 | 34.926 | 1.00 | 50.33 | C |
| ATOM | 11366 | CE1 | PHE | B | 341 | 22.791 | 53.291 | 34.908 | 1.00 | 47.28 | C |
| ATOM | 11368 | CZ | PHE | B | 341 | 23.719 | 52.688 | 35.724 | 1.00 | 49.94 | C |
| ATOM | 11370 | CE2 | PHE | B | 341 | 24.525 | 53.452 | 36.534 | 1.00 | 45.50 | C |
| ATOM | 11372 | CD2 | PHE | B | 341 | 24.408 | 54.811 | 36.540 | 1.00 | 44.62 | C |
| ATOM | 11374 | C | PHE | B | 341 | 22.469 | 58.975 | 36.861 | 1.00 | 47.63 | C |
| ATOM | 11375 | O | PHE | B | 341 | 21.482 | 59.485 | 36.344 | 1.00 | 50.69 | O |
| ATOM | 11376 | N | TYR | B | 342 | 23.470 | 59.686 | 37.372 | 1.00 | 44.30 | N |
| ATOM | 11378 | CA | TYR | B | 342 | 23.694 | 61.096 | 37.030 | 1.00 | 45.47 | C |
| ATOM | 11380 | CB | TYR | B | 342 | 24.640 | 61.771 | 38.026 | 1.00 | 43.43 | C |
| ATOM | 11383 | CG | TYR | B | 342 | 24.793 | 63.285 | 37.896 | 1.00 | 41.05 | C |
| ATOM | 11384 | CD1 | TYR | B | 342 | 23.724 | 64.092 | 37.871 | 1.00 | 40.92 | C |
| ATOM | 11386 | CE1 | TYR | B | 342 | 23.860 | 65.448 | 37.770 | 1.00 | 46.50 | C |
| ATOM | 11388 | CZ | TYR | B | 342 | 25.084 | 66.025 | 37.693 | 1.00 | 49.06 | C |
| ATOM | 11389 | OH | TYR | B | 342 | 25.178 | 67.402 | 37.584 | 1.00 | 46.57 | O |
| ATOM | 11391 | CE2 | TYR | B | 342 | 26.186 | 65.239 | 37.731 | 1.00 | 47.37 | C |
| ATOM | 11393 | CD2 | TYR | B | 342 | 26.036 | 63.881 | 37.832 | 1.00 | 48.16 | C |
| ATOM | 11395 | C | TYR | B | 342 | 24.419 | 60.996 | 35.706 | 1.00 | 42.55 | C |
| ATOM | 11396 | O | TYR | B | 342 | 25.389 | 60.268 | 35.650 | 1.00 | 46.12 | O |
| ATOM | 11397 | N | VAL | B | 343 | 23.952 | 61.690 | 34.665 | 1.00 | 41.14 | N |
| ATOM | 11399 | CA | VAL | B | 343 | 24.592 | 61.717 | 33.333 | 1.00 | 38.62 | C |
| ATOM | 11401 | CB | VAL | B | 343 | 23.618 | 61.281 | 32.277 | 1.00 | 38.70 | C |
| ATOM | 11403 | CG1 | VAL | B | 343 | 24.369 | 61.080 | 30.981 | 1.00 | 40.70 | C |
| ATOM | 11407 | CG2 | VAL | B | 343 | 22.943 | 59.960 | 32.703 | 1.00 | 42.16 | C |
| ATOM | 11411 | C | VAL | B | 343 | 25.070 | 63.099 | 32.869 | 1.00 | 36.25 | C |
| ATOM | 11412 | O | VAL | B | 343 | 24.319 | 64.079 | 32.905 | 1.00 | 43.95 | O |
| ATOM | 11413 | N | VAL | B | 344 | 26.305 | 63.192 | 32.393 | 1.00 | 37.32 | N |
| ATOM | 11415 | CA | VAL | B | 344 | 26.868 | 64.477 | 31.990 | 1.00 | 29.38 | C |
| ATOM | 11417 | CB | VAL | B | 344 | 28.183 | 64.693 | 32.583 | 1.00 | 30.42 | C |
| ATOM | 11419 | CG1 | VAL | B | 344 | 28.516 | 66.115 | 32.541 | 1.00 | 36.77 | C |
| ATOM | 11423 | CG2 | VAL | B | 344 | 28.156 | 64.296 | 34.036 | 1.00 | 38.24 | C |
| ATOM | 11427 | C | VAL | B | 344 | 27.087 | 64.507 | 30.545 | 1.00 | 31.36 | C |
| ATOM | 11428 | O | VAL | B | 344 | 27.722 | 63.618 | 30.003 | 1.00 | 36.95 | O |
| ATOM | 11429 | N | PHE | B | 345 | 26.543 | 65.555 | 29.935 | 1.00 | 38.83 | N |
| ATOM | 11431 | CA | PHE | B | 345 | 26.611 | 65.855 | 28.494 | 1.00 | 41.14 | C |
| ATOM | 11433 | CB | PHE | B | 345 | 25.217 | 66.193 | 27.962 | 1.00 | 41.42 | C |
| ATOM | 11436 | CG | PHE | B | 345 | 24.189 | 65.146 | 28.278 | 1.00 | 44.84 | C |
| ATOM | 11437 | CD1 | PHE | B | 345 | 23.861 | 64.165 | 27.366 | 1.00 | 42.09 | C |
| ATOM | 11439 | CE1 | PHE | B | 345 | 22.953 | 63.237 | 27.669 | 1.00 | 34.11 | C |
| ATOM | 11441 | CZ | PHE | B | 345 | 22.320 | 63.238 | 28.870 | 1.00 | 41.08 | C |
| ATOM | 11443 | CE2 | PHE | B | 345 | 22.617 | 64.181 | 29.785 | 1.00 | 46.16 | C |
| ATOM | 11445 | CD2 | PHE | B | 345 | 23.551 | 65.136 | 29.495 | 1.00 | 43.84 | C |
| ATOM | 11447 | C | PHE | B | 345 | 27.590 | 67.007 | 28.227 | 1.00 | 44.97 | C |
| ATOM | 11448 | O | PHE | B | 345 | 27.217 | 68.145 | 28.007 | 1.00 | 43.88 | O |
| ATOM | 11449 | N | ASP | B | 346 | 28.852 | 66.609 | 28.241 | 1.00 | 47.40 | N |
| ATOM | 11451 | CA | ASP | B | 346 | 30.032 | 67.436 | 28.149 | 1.00 | 48.51 | C |
| ATOM | 11453 | CB | ASP | B | 346 | 31.139 | 66.623 | 28.833 | 1.00 | 52.73 | C |
| ATOM | 11456 | CG | ASP | B | 346 | 32.313 | 67.433 | 29.321 | 1.00 | 60.43 | C |
| ATOM | 11457 | OD1 | ASP | B | 346 | 32.437 | 68.640 | 28.982 | 1.00 | 69.63 | O |
| ATOM | 11458 | OD2 | ASP | B | 346 | 33.173 | 66.892 | 30.074 | 1.00 | 54.36 | O |
| ATOM | 11459 | C | ASP | B | 346 | 30.301 | 67.629 | 26.668 | 1.00 | 47.09 | C |
| ATOM | 11460 | O | ASP | B | 346 | 31.160 | 66.996 | 26.076 | 1.00 | 45.68 | O |
| ATOM | 11461 | N | ARG | B | 347 | 29.506 | 68.517 | 26.081 | 1.00 | 47.52 | N |
| ATOM | 11463 | CA | ARG | B | 347 | 29.637 | 68.935 | 24.701 | 1.00 | 43.73 | C |
| ATOM | 11465 | CB | ARG | B | 347 | 28.570 | 69.962 | 24.423 | 1.00 | 43.30 | C |
| ATOM | 11468 | CG | ARG | B | 347 | 27.202 | 69.356 | 24.324 | 1.00 | 42.11 | C |
| ATOM | 11471 | CD | ARG | B | 347 | 26.079 | 70.326 | 24.660 | 1.00 | 46.02 | C |
| ATOM | 11474 | NE | ARG | B | 347 | 25.906 | 71.409 | 23.697 | 1.00 | 46.95 | N |
| ATOM | 11476 | CZ | ARG | B | 347 | 25.579 | 71.248 | 22.418 | 1.00 | 49.44 | C |
| ATOM | 11477 | NH1 | ARG | B | 347 | 25.401 | 70.035 | 21.910 | 1.00 | 48.15 | N |
| ATOM | 11480 | NH2 | ARG | B | 347 | 25.433 | 72.313 | 21.632 | 1.00 | 51.53 | N |
| ATOM | 11483 | C | ARG | B | 347 | 30.988 | 69.558 | 24.396 | 1.00 | 48.71 | C |
| ATOM | 11484 | O | ARG | B | 347 | 31.492 | 69.455 | 23.286 | 1.00 | 49.46 | O |
| ATOM | 11485 | N | ALA | B | 348 | 31.578 | 70.217 | 25.379 | 1.00 | 49.23 | N |
| ATOM | 11487 | CA | ALA | B | 348 | 32.842 | 70.875 | 25.172 | 1.00 | 46.88 | C |
| ATOM | 11489 | CB | ALA | B | 348 | 33.226 | 71.682 | 26.425 | 1.00 | 48.10 | C |
| ATOM | 11493 | C | ALA | B | 348 | 33.901 | 69.837 | 24.849 | 1.00 | 53.46 | C |
| ATOM | 11494 | O | ALA | B | 348 | 34.834 | 70.103 | 24.076 | 1.00 | 54.08 | O |
| ATOM | 11495 | N | ARG | B | 349 | 33.768 | 68.641 | 25.426 | 1.00 | 54.97 | N |
| ATOM | 11497 | CA | ARG | B | 349 | 34.773 | 67.599 | 25.178 | 1.00 | 51.50 | C |
| ATOM | 11499 | CB | ARG | B | 349 | 35.395 | 67.153 | 26.517 | 1.00 | 50.57 | C |
| ATOM | 11502 | CG | ARG | B | 349 | 36.426 | 68.192 | 27.060 | 1.00 | 50.96 | C |
| ATOM | 11505 | CD | ARG | B | 349 | 36.963 | 67.948 | 28.466 | 1.00 | 53.35 | C |
| ATOM | 11508 | NE | ARG | B | 349 | 37.988 | 66.891 | 28.506 | 1.00 | 58.63 | N |
| ATOM | 11510 | CZ | ARG | B | 349 | 38.667 | 66.554 | 29.599 | 1.00 | 60.78 | C |
| ATOM | 11511 | NH1 | ARG | B | 349 | 38.427 | 67.189 | 30.747 | 1.00 | 54.92 | N |
| ATOM | 11514 | NH2 | ARG | B | 349 | 39.585 | 65.593 | 29.545 | 1.00 | 61.55 | N |
| ATOM | 11517 | C | ARG | B | 349 | 34.290 | 66.413 | 24.305 | 1.00 | 50.33 | C |
| ATOM | 11518 | O | ARG | B | 349 | 34.994 | 65.398 | 24.150 | 1.00 | 50.96 | O |
| ATOM | 11519 | N | LYS | B | 350 | 33.103 | 66.532 | 23.724 | 1.00 | 46.02 | N |
| ATOM | 11521 | CA | LYS | B | 350 | 32.610 | 65.465 | 22.881 | 1.00 | 46.35 | C |
| ATOM | 11523 | CB | LYS | B | 350 | 33.496 | 65.367 | 21.601 | 1.00 | 49.69 | C |
| ATOM | 11526 | CG | LYS | B | 350 | 33.014 | 64.434 | 20.402 | 1.00 | 58.03 | C |
| ATOM | 11529 | CD | LYS | B | 350 | 33.785 | 64.714 | 19.003 | 1.00 | 58.14 | C |
| ATOM | 11532 | CE | LYS | B | 350 | 33.246 | 63.860 | 17.760 | 1.00 | 60.61 | C |
| ATOM | 11535 | NZ | LYS | B | 350 | 33.683 | 64.245 | 16.314 | 1.00 | 56.11 | N |
| ATOM | 11539 | C | LYS | B | 350 | 32.554 | 64.164 | 23.717 | 1.00 | 44.86 | C |
| ATOM | 11540 | O | LYS | B | 350 | 32.810 | 63.100 | 23.206 | 1.00 | 48.76 | O |
| ATOM | 11541 | N | ARG | B | 351 | 32.200 | 64.257 | 25.007 | 1.00 | 44.60 | N |
| ATOM | 11543 | CA | ARG | B | 351 | 32.015 | 63.060 | 25.863 | 1.00 | 44.48 | C |
| ATOM | 11545 | CB | ARG | B | 351 | 33.225 | 62.846 | 26.743 | 1.00 | 43.82 | C |
| ATOM | 11548 | CG | ARG | B | 351 | 33.540 | 64.024 | 27.571 | 1.00 | 45.18 | C |
| ATOM | 11551 | CD | ARG | B | 351 | 34.789 | 63.898 | 28.392 | 1.00 | 40.95 | C |
| ATOM | 11554 | NE | ARG | B | 351 | 34.687 | 64.715 | 29.598 | 1.00 | 46.55 | N |
| ATOM | 11556 | CZ | ARG | B | 351 | 35.630 | 64.793 | 30.519 | 1.00 | 43.31 | C |
| ATOM | 11557 | NH1 | ARG | B | 351 | 36.756 | 64.127 | 30.377 | 1.00 | 41.89 | N |
| ATOM | 11560 | NH2 | ARG | B | 351 | 35.456 | 65.547 | 31.590 | 1.00 | 54.20 | N |
| ATOM | 11563 | C | ARG | B | 351 | 30.791 | 63.069 | 26.789 | 1.00 | 41.64 | C |
| ATOM | 11564 | O | ARG | B | 351 | 30.368 | 64.121 | 27.205 | 1.00 | 50.18 | O |
| ATOM | 11565 | N | ILE | B | 352 | 30.257 | 61.883 | 27.104 | 1.00 | 38.69 | N |
| ATOM | 11567 | CA | ILE | B | 352 | 29.130 | 61.673 | 28.027 | 1.00 | 45.01 | C |
| ATOM | 11569 | CB | ILE | B | 352 | 28.098 | 60.731 | 27.416 | 1.00 | 49.66 | C |
| ATOM | 11571 | CG1 | ILE | B | 352 | 27.315 | 61.409 | 26.305 | 1.00 | 52.66 | C |
| ATOM | 11574 | CD1 | ILE | B | 352 | 26.048 | 60.706 | 26.013 | 1.00 | 56.74 | C |
| ATOM | 11578 | CG2 | ILE | B | 352 | 27.127 | 60.305 | 28.506 | 1.00 | 52.33 | C |
| ATOM | 11582 | C | ILE | B | 352 | 29.550 | 60.932 | 29.289 | 1.00 | 46.49 | C |
| ATOM | 11583 | O | ILE | B | 352 | 30.129 | 59.841 | 29.192 | 1.00 | 45.55 | O |
| ATOM | 11584 | N | GLY | B | 353 | 29.237 | 61.448 | 30.471 | 1.00 | 46.26 | N |
| ATOM | 11586 | CA | GLY | B | 353 | 29.635 | 60.734 | 31.670 | 1.00 | 44.22 | C |
| ATOM | 11589 | C | GLY | B | 353 | 28.530 | 60.101 | 32.472 | 1.00 | 40.63 | C |
| ATOM | 11590 | O | GLY | B | 353 | 27.454 | 60.641 | 32.543 | 1.00 | 50.25 | O |
| ATOM | 11591 | N | PHE | B | 354 | 28.815 | 58.960 | 33.096 | 1.00 | 41.57 | N |
| ATOM | 11593 | CA | PHE | B | 354 | 27.860 | 58.254 | 33.937 | 1.00 | 35.69 | C |
| ATOM | 11595 | CB | PHE | B | 354 | 27.614 | 56.866 | 33.365 | 1.00 | 42.54 | C |
| ATOM | 11598 | CG | PHE | B | 354 | 27.032 | 56.890 | 31.992 | 1.00 | 48.12 | C |
| ATOM | 11599 | CD1 | PHE | B | 354 | 27.843 | 57.064 | 30.896 | 1.00 | 47.79 | C |
| ATOM | 11601 | CE1 | PHE | B | 354 | 27.311 | 57.107 | 29.652 | 1.00 | 53.15 | C |
| ATOM | 11603 | CZ | PHE | B | 354 | 25.957 | 56.971 | 29.486 | 1.00 | 51.31 | C |
| ATOM | 11605 | CE2 | PHE | B | 354 | 25.146 | 56.790 | 30.572 | 1.00 | 45.30 | C |
| ATOM | 11607 | CD2 | PHE | B | 354 | 25.670 | 56.757 | 31.809 | 1.00 | 42.69 | C |
| ATOM | 11609 | C | PHE | B | 354 | 28.418 | 58.065 | 35.324 | 1.00 | 37.73 | C |
| ATOM | 11610 | O | PHE | B | 354 | 29.561 | 57.648 | 35.479 | 1.00 | 36.56 | O |
| ATOM | 11611 | N | ALA | B | 355 | 27.611 | 58.364 | 36.333 | 1.00 | 34.99 | N |
| ATOM | 11613 | CA | ALA | B | 355 | 27.973 | 58.081 | 37.695 | 1.00 | 31.85 | C |
| ATOM | 11615 | CB | ALA | B | 355 | 28.631 | 59.305 | 38.285 | 1.00 | 35.10 | C |
| ATOM | 11619 | C | ALA | B | 355 | 26.740 | 57.630 | 38.531 | 1.00 | 34.75 | C |
| ATOM | 11620 | O | ALA | B | 355 | 25.641 | 58.105 | 38.363 | 1.00 | 38.79 | O |
| ATOM | 11621 | N | VAL | B | 356 | 26.909 | 56.686 | 39.430 | 1.00 | 36.60 | N |
| ATOM | 11623 | CA | VAL | B | 356 | 25.801 | 56.261 | 40.272 | 1.00 | 35.07 | C |
| ATOM | 11625 | CB | VAL | B | 356 | 26.370 | 55.348 | 41.344 | 1.00 | 32.99 | C |
| ATOM | 11627 | CG1 | VAL | B | 356 | 25.358 | 55.012 | 42.383 | 1.00 | 38.64 | C |
| ATOM | 11631 | CG2 | VAL | B | 356 | 26.829 | 54.112 | 40.710 | 1.00 | 35.78 | C |
| ATOM | 11635 | C | VAL | B | 356 | 25.077 | 57.477 | 40.879 | 1.00 | 35.09 | C |
| ATOM | 11636 | O | VAL | B | 356 | 25.717 | 58.401 | 41.338 | 1.00 | 40.54 | O |
| ATOM | 11637 | N | SER | B | 357 | 23.752 | 57.483 | 40.905 | 1.00 | 41.74 | N |
| ATOM | 11639 | CA | SER | B | 357 | 22.999 | 58.670 | 41.346 | 1.00 | 41.57 | C |
| ATOM | 11641 | CB | SER | B | 357 | 21.721 | 58.803 | 40.523 | 1.00 | 47.83 | C |
| ATOM | 11644 | OG | SER | B | 357 | 21.001 | 60.008 | 40.844 | 1.00 | 50.14 | O |
| ATOM | 11646 | C | SER | B | 357 | 22.591 | 58.706 | 42.805 | 1.00 | 44.92 | C |
| ATOM | 11647 | O | SER | B | 357 | 22.032 | 57.767 | 43.335 | 1.00 | 49.30 | O |
| ATOM | 11648 | N | ALA | B | 358 | 22.822 | 59.824 | 43.458 | 1.00 | 47.90 | N |
| ATOM | 11650 | CA | ALA | B | 358 | 22.455 | 59.955 | 44.856 | 1.00 | 50.90 | C |
| ATOM | 11652 | CB | ALA | B | 358 | 22.871 | 61.301 | 45.346 | 1.00 | 52.95 | C |
| ATOM | 11656 | C | ALA | B | 358 | 20.962 | 59.764 | 45.155 | 1.00 | 51.41 | C |
| ATOM | 11657 | O | ALA | B | 358 | 20.538 | 59.955 | 46.293 | 1.00 | 50.45 | O |
| ATOM | 11658 | N | CYS | B | 359 | 20.158 | 59.408 | 44.167 | 1.00 | 51.99 | N |
| ATOM | 11660 | CA | CYS | B | 359 | 18.739 | 59.237 | 44.444 | 1.00 | 56.13 | C |
| ATOM | 11662 | CB | CYS | B | 359 | 17.928 | 60.518 | 44.118 | 1.00 | 57.03 | C |
| ATOM | 11665 | SG | CYS | B | 359 | 17.612 | 60.710 | 42.327 | 1.00 | 70.47 | S |
| ATOM | 11666 | C | CYS | B | 359 | 18.122 | 58.055 | 43.726 | 1.00 | 51.20 | C |
| ATOM | 11667 | O | CYS | B | 359 | 16.907 | 58.001 | 43.614 | 1.00 | 57.81 | O |
| ATOM | 11668 | N | HIS | B | 360 | 18.909 | 57.107 | 43.238 | 1.00 | 46.00 | N |
| ATOM | 11670 | CA | HIS | B | 360 | 18.286 | 55.955 | 42.602 | 1.00 | 46.33 | C |
| ATOM | 11672 | CB | HIS | B | 360 | 19.256 | 55.165 | 41.736 | 1.00 | 43.36 | C |
| ATOM | 11675 | CG | HIS | B | 360 | 20.215 | 54.331 | 42.501 | 1.00 | 38.68 | C |
| ATOM | 11676 | ND1 | HIS | B | 360 | 21.398 | 54.828 | 42.996 | 1.00 | 40.71 | N |
| ATOM | 11678 | CE1 | HIS | B | 360 | 22.052 | 53.864 | 43.613 | 1.00 | 38.06 | C |
| ATOM | 11680 | NE2 | HIS | B | 360 | 21.334 | 52.758 | 43.535 | 1.00 | 40.34 | N |
| ATOM | 11682 | CD2 | HIS | B | 360 | 20.178 | 53.030 | 42.847 | 1.00 | 39.93 | C |
| ATOM | 11684 | C | HIS | B | 360 | 17.608 | 55.028 | 43.604 | 1.00 | 47.19 | C |
| ATOM | 11685 | O | HIS | B | 360 | 18.049 | 54.855 | 44.722 | 1.00 | 49.29 | O |
| ATOM | 11686 | N | VAL | B | 361 | 16.521 | 54.430 | 43.157 | 1.00 | 49.14 | N |
| ATOM | 11688 | CA | VAL | B | 361 | 15.712 | 53.536 | 43.966 | 1.00 | 49.38 | C |
| ATOM | 11690 | CB | VAL | B | 361 | 14.359 | 53.315 | 43.229 | 1.00 | 48.93 | C |
| ATOM | 11692 | CG1 | VAL | B | 361 | 13.659 | 52.119 | 43.747 | 1.00 | 50.27 | C |
| ATOM | 11696 | CG2 | VAL | B | 361 | 13.474 | 54.527 | 43.327 | 1.00 | 49.22 | C |
| ATOM | 11700 | C | VAL | B | 361 | 16.427 | 52.201 | 44.061 | 1.00 | 48.86 | C |
| ATOM | 11701 | O | VAL | B | 361 | 16.805 | 51.659 | 43.055 | 1.00 | 50.03 | O |
| ATOM | 11702 | N | HIS | B | 362 | 16.636 | 51.656 | 45.235 | 1.00 | 50.40 | N |
| ATOM | 11704 | CA | HIS | B | 362 | 17.322 | 50.373 | 45.323 | 1.00 | 54.92 | C |
| ATOM | 11706 | CB | HIS | B | 362 | 18.864 | 50.539 | 45.254 | 1.00 | 54.49 | C |
| ATOM | 11709 | CG | HIS | B | 362 | 19.377 | 51.443 | 46.321 | 1.00 | 60.33 | C |
| ATOM | 11710 | ND1 | HIS | B | 362 | 19.017 | 52.771 | 46.390 | 1.00 | 66.26 | N |
| ATOM | 11712 | CE1 | HIS | B | 362 | 19.596 | 53.328 | 47.442 | 1.00 | 65.09 | C |
| ATOM | 11714 | NE2 | HIS | B | 362 | 20.305 | 52.406 | 48.065 | 1.00 | 61.43 | N |
| ATOM | 11716 | CD2 | HIS | B | 362 | 20.174 | 51.216 | 47.389 | 1.00 | 61.57 | C |
| ATOM | 11718 | C | HIS | B | 362 | 16.929 | 49.742 | 46.671 | 1.00 | 54.45 | C |
| ATOM | 11719 | O | HIS | B | 362 | 16.069 | 50.242 | 47.362 | 1.00 | 53.82 | O |
| ATOM | 11720 | N | ASP | B | 363 | 17.564 | 48.650 | 47.059 | 1.00 | 53.43 | N |
| ATOM | 11722 | CA | ASP | B | 363 | 17.217 | 48.054 | 48.320 | 1.00 | 54.67 | C |
| ATOM | 11724 | CB | ASP | B | 363 | 16.494 | 46.752 | 48.043 | 1.00 | 56.65 | C |
| ATOM | 11727 | CG | ASP | B | 363 | 17.334 | 45.830 | 47.255 | 1.00 | 56.00 | C |
| ATOM | 11728 | OD1 | ASP | B | 363 | 18.346 | 45.373 | 47.833 | 1.00 | 60.63 | O |
| ATOM | 11729 | OD2 | ASP | B | 363 | 17.083 | 45.532 | 46.063 | 1.00 | 51.94 | O |
| ATOM | 11730 | C | ASP | B | 363 | 18.443 | 47.756 | 49.171 | 1.00 | 55.74 | C |
| ATOM | 11731 | O | ASP | B | 363 | 19.562 | 48.178 | 48.890 | 1.00 | 54.34 | O |
| ATOM | 11732 | N | GLU | B | 364 | 18.180 | 46.998 | 50.216 | 1.00 | 54.75 | N |
| ATOM | 11734 | CA | GLU | B | 364 | 19.153 | 46.627 | 51.197 | 1.00 | 58.19 | C |
| ATOM | 11736 | CB | GLU | B | 364 | 18.486 | 45.696 | 52.252 | 1.00 | 66.95 | C |
| ATOM | 11739 | CG | GLU | B | 364 | 17.379 | 44.737 | 51.746 | 1.00 | 73.62 | C |
| ATOM | 11742 | CD | GLU | B | 364 | 15.939 | 45.284 | 51.760 | 1.00 | 79.77 | C |
| ATOM | 11743 | OE1 | GLU | B | 364 | 15.620 | 46.193 | 50.945 | 1.00 | 81.19 | O |
| ATOM | 11744 | OE2 | GLU | B | 364 | 15.110 | 44.780 | 52.581 | 1.00 | 79.52 | O |
| ATOM | 11745 | C | GLU | B | 364 | 20.436 | 46.015 | 50.655 | 1.00 | 53.23 | C |
| ATOM | 11746 | O | GLU | B | 364 | 21.497 | 46.365 | 51.142 | 1.00 | 62.76 | O |
| ATOM | 11747 | N | PHE | B | 365 | 20.359 | 45.146 | 49.652 | 1.00 | 48.86 | N |
| ATOM | 11749 | CA | PHE | B | 365 | 21.509 | 44.351 | 49.213 | 1.00 | 45.25 | C |
| ATOM | 11751 | CB | PHE | B | 365 | 21.180 | 42.866 | 49.376 | 1.00 | 47.71 | C |
| ATOM | 11754 | CG | PHE | B | 365 | 20.355 | 42.527 | 50.568 | 1.00 | 47.10 | C |
| ATOM | 11755 | CD1 | PHE | B | 365 | 19.002 | 42.462 | 50.470 | 1.00 | 48.77 | C |
| ATOM | 11757 | CE1 | PHE | B | 365 | 18.227 | 42.134 | 51.573 | 1.00 | 50.48 | C |
| ATOM | 11759 | CZ | PHE | B | 365 | 18.807 | 41.860 | 52.769 | 1.00 | 50.39 | C |
| ATOM | 11761 | CE2 | PHE | B | 365 | 20.163 | 41.906 | 52.892 | 1.00 | 49.25 | C |
| ATOM | 11763 | CD2 | PHE | B | 365 | 20.941 | 42.236 | 51.784 | 1.00 | 52.57 | C |
| ATOM | 11765 | C | PHE | B | 365 | 22.028 | 44.401 | 47.758 | 1.00 | 48.01 | C |
| ATOM | 11766 | O | PHE | B | 365 | 22.790 | 43.488 | 47.355 | 1.00 | 48.65 | O |
| ATOM | 11767 | N | ARG | B | 366 | 21.628 | 45.387 | 46.959 | 1.00 | 41.71 | N |
| ATOM | 11769 | CA | ARG | B | 366 | 22.110 | 45.495 | 45.595 | 1.00 | 38.21 | C |
| ATOM | 11771 | CB | ARG | B | 366 | 21.196 | 44.800 | 44.631 | 1.00 | 44.13 | C |
| ATOM | 11774 | CG | ARG | B | 366 | 21.366 | 43.373 | 44.547 | 1.00 | 45.53 | C |
| ATOM | 11777 | CD | ARG | B | 366 | 21.069 | 42.891 | 43.182 | 1.00 | 47.33 | C |
| ATOM | 11780 | NE | ARG | B | 366 | 19.640 | 42.980 | 42.943 | 1.00 | 46.43 | N |
| ATOM | 11782 | CZ | ARG | B | 366 | 18.989 | 42.079 | 42.253 | 1.00 | 48.01 | C |
| ATOM | 11783 | NH1 | ARG | B | 366 | 19.682 | 41.061 | 41.765 | 1.00 | 42.06 | N |
| ATOM | 11786 | NH2 | ARG | B | 366 | 17.671 | 42.188 | 42.048 | 1.00 | 48.96 | N |
| ATOM | 11789 | C | ARG | B | 366 | 21.978 | 46.909 | 45.262 | 1.00 | 39.85 | C |
| ATOM | 11790 | O | ARG | B | 366 | 21.091 | 47.555 | 45.778 | 1.00 | 43.10 | O |
| ATOM | 11791 | N | THR | B | 367 | 22.812 | 47.388 | 44.353 | 1.00 | 43.90 | N |
| ATOM | 11793 | CA | THR | B | 367 | 22.890 | 48.806 | 44.093 | 1.00 | 45.12 | C |
| ATOM | 11795 | CB | THR | B | 367 | 23.905 | 49.378 | 45.030 | 1.00 | 48.16 | C |
| ATOM | 11797 | OG1 | THR | B | 367 | 23.712 | 50.781 | 45.171 | 1.00 | 51.47 | O |
| ATOM | 11799 | CG2 | THR | B | 367 | 25.310 | 49.228 | 44.422 | 1.00 | 49.76 | C |
| ATOM | 11803 | C | THR | B | 367 | 23.342 | 49.094 | 42.705 | 1.00 | 44.62 | C |
| ATOM | 11804 | O | THR | B | 367 | 24.218 | 48.423 | 42.196 | 1.00 | 54.55 | O |
| ATOM | 11805 | N | ALA | B | 368 | 22.734 | 50.097 | 42.088 | 1.00 | 49.08 | N |
| ATOM | 11807 | CA | ALA | B | 368 | 23.096 | 50.539 | 40.742 | 1.00 | 46.61 | C |
| ATOM | 11809 | CB | ALA | B | 368 | 22.408 | 51.789 | 40.479 | 1.00 | 48.60 | C |
| ATOM | 11813 | C | ALA | B | 368 | 24.589 | 50.768 | 40.715 | 1.00 | 47.51 | C |
| ATOM | 11814 | O | ALA | B | 368 | 25.131 | 51.084 | 41.752 | 1.00 | 54.43 | O |
| ATOM | 11815 | N | ALA | B | 369 | 25.248 | 50.662 | 39.561 | 1.00 | 46.20 | N |
| ATOM | 11817 | CA | ALA | B | 369 | 26.685 | 50.690 | 39.528 | 1.00 | 45.00 | C |
| ATOM | 11819 | CB | ALA | B | 369 | 27.158 | 49.328 | 40.018 | 1.00 | 47.24 | C |
| ATOM | 11823 | C | ALA | B | 369 | 27.496 | 51.050 | 38.249 | 1.00 | 44.87 | C |
| ATOM | 11824 | O | ALA | B | 369 | 27.161 | 50.643 | 37.116 | 1.00 | 48.30 | O |
| ATOM | 11825 | N | VAL | B | 370 | 28.620 | 51.759 | 38.447 | 1.00 | 42.20 | N |
| ATOM | 11827 | CA | VAL | B | 370 | 29.468 | 52.125 | 37.315 | 1.00 | 38.93 | C |
| ATOM | 11829 | CB | VAL | B | 370 | 29.495 | 53.628 | 37.014 | 1.00 | 36.63 | C |
| ATOM | 11831 | CG1 | VAL | B | 370 | 30.429 | 53.873 | 35.921 | 1.00 | 41.13 | C |
| ATOM | 11835 | CG2 | VAL | B | 370 | 28.134 | 54.161 | 36.602 | 1.00 | 37.20 | C |
| ATOM | 11839 | C | VAL | B | 370 | 30.838 | 51.685 | 37.636 | 1.00 | 38.61 | C |
| ATOM | 11840 | O | VAL | B | 370 | 31.420 | 52.204 | 38.525 | 1.00 | 38.62 | O |
| ATOM | 11841 | N | GLU | B | 371 | 31.390 | 50.748 | 36.885 | 1.00 | 43.13 | N |
| ATOM | 11843 | CA | GLU | B | 371 | 32.643 | 50.189 | 37.276 | 1.00 | 41.26 | C |
| ATOM | 11845 | CB | GLU | B | 371 | 32.358 | 48.778 | 37.822 | 1.00 | 43.99 | C |
| ATOM | 11848 | CG | GLU | B | 371 | 31.606 | 48.790 | 39.157 | 1.00 | 46.24 | C |
| ATOM | 11851 | CD | GLU | B | 371 | 31.166 | 47.419 | 39.677 | 1.00 | 51.18 | C |
| ATOM | 11852 | OE1 | GLU | B | 371 | 31.380 | 47.199 | 40.880 | 1.00 | 54.14 | O |
| ATOM | 11853 | OE2 | GLU | B | 371 | 30.602 | 46.566 | 38.927 | 1.00 | 53.22 | O |
| ATOM | 11854 | C | GLU | B | 371 | 33.499 | 50.071 | 36.085 | 1.00 | 43.69 | C |
| ATOM | 11855 | O | GLU | B | 371 | 32.935 | 49.921 | 35.040 | 1.00 | 40.57 | O |
| ATOM | 11856 | N | GLY | B | 372 | 34.842 | 50.169 | 36.240 | 1.00 | 49.03 | N |
| ATOM | 11858 | CA | GLY | B | 372 | 35.850 | 49.814 | 35.201 | 1.00 | 46.30 | C |
| ATOM | 11861 | C | GLY | B | 372 | 37.272 | 49.955 | 35.731 | 1.00 | 47.83 | C |
| ATOM | 11862 | O | GLY | B | 372 | 37.433 | 50.174 | 36.939 | 1.00 | 53.24 | O |
| ATOM | 11863 | N | PRO | B | 373 | 38.339 | 49.869 | 34.922 | 1.00 | 48.85 | N |
| ATOM | 11864 | CA | PRO | B | 373 | 38.364 | 49.535 | 33.488 | 1.00 | 47.06 | C |
| ATOM | 11866 | CB | PRO | B | 373 | 39.716 | 50.086 | 33.023 | 1.00 | 44.96 | C |
| ATOM | 11869 | CG | PRO | B | 373 | 40.533 | 49.940 | 34.115 | 1.00 | 43.41 | C |
| ATOM | 11872 | CD | PRO | B | 373 | 39.682 | 50.230 | 35.387 | 1.00 | 43.08 | C |
| ATOM | 11875 | C | PRO | B | 373 | 38.491 | 48.092 | 33.219 | 1.00 | 50.22 | C |
| ATOM | 11876 | O | PRO | B | 373 | 39.130 | 47.394 | 33.964 | 1.00 | 57.42 | O |
| ATOM | 11877 | N | PHE | B | 374 | 37.903 | 47.658 | 32.137 | 1.00 | 51.76 | N |
| ATOM | 11879 | CA | PHE | B | 374 | 38.030 | 46.319 | 31.713 | 1.00 | 53.93 | C |
| ATOM | 11881 | CB | PHE | B | 374 | 36.648 | 45.693 | 31.494 | 1.00 | 56.43 | C |
| ATOM | 11884 | CG | PHE | B | 374 | 35.736 | 45.873 | 32.661 | 1.00 | 53.63 | C |
| ATOM | 11885 | CD1 | PHE | B | 374 | 35.069 | 47.063 | 32.842 | 1.00 | 54.53 | C |
| ATOM | 11887 | CE1 | PHE | B | 374 | 34.249 | 47.242 | 33.904 | 1.00 | 56.48 | C |
| ATOM | 11889 | CZ | PHE | B | 374 | 34.093 | 46.226 | 34.821 | 1.00 | 57.87 | C |
| ATOM | 11891 | CE2 | PHE | B | 374 | 34.758 | 45.038 | 34.654 | 1.00 | 50.55 | C |
| ATOM | 11893 | CD2 | PHE | B | 374 | 35.572 | 44.865 | 33.579 | 1.00 | 49.47 | C |
| ATOM | 11895 | C | PHE | B | 374 | 38.736 | 46.536 | 30.420 | 1.00 | 59.86 | C |
| ATOM | 11896 | O | PHE | B | 374 | 38.433 | 47.466 | 29.694 | 1.00 | 62.61 | O |
| ATOM | 11897 | N | VAL | B | 375 | 39.692 | 45.677 | 30.129 | 1.00 | 68.35 | N |
| ATOM | 11899 | CA | VAL | B | 375 | 40.369 | 45.737 | 28.857 | 1.00 | 68.00 | C |
| ATOM | 11901 | CB | VAL | B | 375 | 41.765 | 45.125 | 28.921 | 1.00 | 69.56 | C |
| ATOM | 11903 | CG1 | VAL | B | 375 | 42.126 | 44.497 | 27.585 | 1.00 | 72.03 | C |
| ATOM | 11907 | CG2 | VAL | B | 375 | 42.774 | 46.194 | 29.334 | 1.00 | 71.07 | C |
| ATOM | 11911 | C | VAL | B | 375 | 39.508 | 45.050 | 27.808 | 1.00 | 72.55 | C |
| ATOM | 11912 | O | VAL | B | 375 | 39.061 | 43.915 | 27.984 | 1.00 | 64.78 | O |
| ATOM | 11913 | N | THR | B | 376 | 39.275 | 45.774 | 26.712 | 1.00 | 81.41 | N |
| ATOM | 11915 | CA | THR | B | 376 | 38.464 | 45.296 | 25.589 | 1.00 | 86.08 | C |
| ATOM | 11917 | CB | THR | B | 376 | 37.142 | 45.981 | 25.640 | 1.00 | 85.71 | C |
| ATOM | 11919 | OG1 | THR | B | 376 | 36.993 | 46.575 | 26.932 | 1.00 | 88.06 | O |
| ATOM | 11921 | CG2 | THR | B | 376 | 35.986 | 44.970 | 25.508 | 1.00 | 86.58 | C |
| ATOM | 11925 | C | THR | B | 376 | 39.130 | 45.590 | 24.246 | 1.00 | 90.80 | C |
| ATOM | 11926 | O | THR | B | 376 | 39.889 | 46.565 | 24.111 | 1.00 | 93.52 | O |
| ATOM | 11927 | N | LEU | B | 377 | 38.840 | 44.768 | 23.240 | 1.00 | 94.98 | N |
| ATOM | 11929 | CA | LEU | B | 377 | 39.572 | 44.884 | 21.974 | 1.00 | 97.54 | C |
| ATOM | 11931 | CB | LEU | B | 377 | 40.491 | 43.667 | 21.839 | 1.00 | 97.95 | C |
| ATOM | 11934 | CG | LEU | B | 377 | 41.234 | 43.239 | 23.119 | 1.00 | 97.10 | C |
| ATOM | 11936 | CD1 | LEU | B | 377 | 42.302 | 42.213 | 22.767 | 1.00 | 96.01 | C |
| ATOM | 11940 | CD2 | LEU | B | 377 | 41.873 | 44.413 | 23.852 | 1.00 | 96.18 | C |
| ATOM | 11944 | C | LEU | B | 377 | 38.777 | 45.053 | 20.662 | 1.00 | 98.30 | C |
| ATOM | 11945 | O | LEU | B | 377 | 37.719 | 44.425 | 20.470 | 1.00 | 95.52 | O |
| ATOM | 11946 | N | ASP | B | 378 | 39.328 | 45.909 | 19.782 | 1.00 | 97.57 | N |
| ATOM | 11948 | CA | ASP | B | 378 | 38.846 | 46.107 | 18.405 | 1.00 | 98.72 | C |
| ATOM | 11950 | CB | ASP | B | 378 | 38.818 | 44.778 | 17.631 | 1.00 | 101.77 | C |
| ATOM | 11953 | CG | ASP | B | 378 | 40.161 | 44.040 | 17.668 | 1.00 | 106.54 | C |
| ATOM | 11954 | OD1 | ASP | B | 378 | 40.185 | 42.823 | 17.348 | 1.00 | 107.57 | O |
| ATOM | 11955 | OD2 | ASP | B | 378 | 41.238 | 44.594 | 18.001 | 1.00 | 108.22 | O |
| ATOM | 11956 | C | ASP | B | 378 | 37.466 | 46.678 | 18.517 | 1.00 | 95.34 | C |
| ATOM | 11957 | O | ASP | B | 378 | 36.576 | 46.473 | 17.690 | 1.00 | 90.09 | O |
| ATOM | 11958 | N | MET | B | 379 | 37.315 | 47.427 | 19.584 | 1.00 | 92.72 | N |
| ATOM | 11960 | CA | MET | B | 379 | 36.018 | 47.850 | 19.970 | 1.00 | 89.67 | C |
| ATOM | 11962 | CB | MET | B | 379 | 36.115 | 48.778 | 21.181 | 1.00 | 87.84 | C |
| ATOM | 11965 | CG | MET | B | 379 | 36.814 | 48.131 | 22.363 | 1.00 | 85.16 | C |
| ATOM | 11968 | SD | MET | B | 379 | 36.294 | 48.757 | 23.944 | 1.00 | 81.06 | S |
| ATOM | 11969 | CE | MET | B | 379 | 37.408 | 49.948 | 24.177 | 1.00 | 82.73 | C |
| ATOM | 11973 | C | MET | B | 379 | 35.455 | 48.535 | 18.782 | 1.00 | 88.14 | C |
| ATOM | 11974 | O | MET | B | 379 | 34.453 | 48.097 | 18.241 | 1.00 | 89.91 | O |
| ATOM | 11975 | N | GLU | B | 380 | 36.133 | 49.590 | 18.349 | 1.00 | 89.97 | N |
| ATOM | 11977 | CA | GLU | B | 380 | 35.555 | 50.483 | 17.361 | 1.00 | 89.95 | C |
| ATOM | 11979 | CB | GLU | B | 380 | 36.642 | 51.168 | 16.508 | 1.00 | 89.88 | C |
| ATOM | 11982 | CG | GLU | B | 380 | 36.336 | 52.614 | 16.072 | 1.00 | 90.98 | C |
| ATOM | 11985 | CD | GLU | B | 380 | 34.854 | 52.999 | 16.089 | 1.00 | 88.44 | C |
| ATOM | 11986 | OE1 | GLU | B | 380 | 34.530 | 54.085 | 16.634 | 1.00 | 82.43 | O |
| ATOM | 11987 | OE2 | GLU | B | 380 | 34.021 | 52.227 | 15.552 | 1.00 | 85.64 | O |
| ATOM | 11988 | C | GLU | B | 380 | 34.529 | 49.668 | 16.547 | 1.00 | 90.22 | C |
| ATOM | 11989 | O | GLU | B | 380 | 33.385 | 50.102 | 16.385 | 1.00 | 89.91 | O |
| ATOM | 11990 | N | ASP | B | 381 | 34.946 | 48.482 | 16.080 | 1.00 | 89.23 | N |
| ATOM | 11992 | CA | ASP | B | 381 | 34.122 | 47.536 | 15.284 | 1.00 | 85.33 | C |
| ATOM | 11994 | CB | ASP | B | 381 | 34.705 | 46.142 | 15.374 | 1.00 | 86.39 | C |
| ATOM | 11997 | CG | ASP | B | 381 | 35.859 | 45.959 | 14.456 | 1.00 | 91.21 | C |
| ATOM | 11998 | OD1 | ASP | B | 381 | 35.821 | 46.563 | 13.353 | 1.00 | 93.25 | O |
| ATOM | 11999 | OD2 | ASP | B | 381 | 36.842 | 45.237 | 14.742 | 1.00 | 97.75 | O |
| ATOM | 12000 | C | ASP | B | 381 | 32.676 | 47.329 | 15.606 | 1.00 | 79.23 | C |
| ATOM | 12001 | O | ASP | B | 381 | 31.926 | 46.925 | 14.734 | 1.00 | 79.04 | O |
| ATOM | 12002 | N | CYS | B | 382 | 32.270 | 47.561 | 16.839 | 1.00 | 73.79 | N |
| ATOM | 12004 | CA | CYS | B | 382 | 30.891 | 47.324 | 17.189 | 1.00 | 72.63 | C |
| ATOM | 12006 | CB | CYS | B | 382 | 30.801 | 47.291 | 18.685 | 1.00 | 73.33 | C |
| ATOM | 12009 | SG | CYS | B | 382 | 32.081 | 46.160 | 19.287 | 1.00 | 72.63 | S |
| ATOM | 12010 | C | CYS | B | 382 | 30.101 | 48.438 | 16.574 | 1.00 | 70.89 | C |
| ATOM | 12011 | O | CYS | B | 382 | 28.891 | 48.364 | 16.388 | 1.00 | 71.67 | O |
| ATOM | 12012 | N | GLY | B | 383 | 30.838 | 49.484 | 16.246 | 1.00 | 72.45 | N |
| ATOM | 12014 | CA | GLY | B | 383 | 30.295 | 50.631 | 15.572 | 1.00 | 71.23 | C |
| ATOM | 12017 | C | GLY | B | 383 | 29.732 | 50.152 | 14.271 | 1.00 | 69.89 | C |
| ATOM | 12018 | O | GLY | B | 383 | 30.294 | 49.291 | 13.612 | 1.00 | 73.05 | O |
| ATOM | 12019 | N | TYR | B | 384 | 28.597 | 50.723 | 13.928 | 1.00 | 67.13 | N |
| ATOM | 12021 | CA | TYR | B | 384 | 27.915 | 50.436 | 12.714 | 1.00 | 64.11 | C |
| ATOM | 12023 | CB | TYR | B | 384 | 26.468 | 50.421 | 13.020 | 1.00 | 61.05 | C |
| ATOM | 12026 | CG | TYR | B | 384 | 25.620 | 50.091 | 11.867 | 1.00 | 59.89 | C |
| ATOM | 12027 | CD1 | TYR | B | 384 | 25.280 | 48.777 | 11.578 | 1.00 | 59.47 | C |
| ATOM | 12029 | CE1 | TYR | B | 384 | 24.476 | 48.469 | 10.515 | 1.00 | 61.28 | C |
| ATOM | 12031 | CZ | TYR | B | 384 | 23.997 | 49.497 | 9.733 | 1.00 | 62.97 | C |
| ATOM | 12032 | OH | TYR | B | 384 | 23.179 | 49.288 | 8.636 | 1.00 | 63.75 | O |
| ATOM | 12034 | CE2 | TYR | B | 384 | 24.332 | 50.792 | 10.028 | 1.00 | 64.64 | C |
| ATOM | 12036 | CD2 | TYR | B | 384 | 25.136 | 51.079 | 11.083 | 1.00 | 60.10 | C |
| ATOM | 12038 | C | TYR | B | 384 | 28.185 | 51.536 | 11.732 | 1.00 | 68.06 | C |
| ATOM | 12039 | O | TYR | B | 384 | 28.365 | 52.708 | 12.106 | 1.00 | 67.06 | O |
| ATOM | 12040 | N | ASN | B | 385 | 28.213 | 51.178 | 10.464 | 1.00 | 71.13 | N |
| ATOM | 12042 | CA | ASN | B | 385 | 28.501 | 52.166 | 9.438 | 1.00 | 74.74 | C |
| ATOM | 12044 | CB | ASN | B | 385 | 29.894 | 51.890 | 8.828 | 1.00 | 76.30 | C |
| ATOM | 12047 | CG | ASN | B | 385 | 31.057 | 51.931 | 9.889 | 1.00 | 78.44 | C |
| ATOM | 12048 | OD1 | ASN | B | 385 | 30.989 | 52.640 | 10.887 | 1.00 | 84.85 | O |
| ATOM | 12049 | ND2 | ASN | B | 385 | 32.111 | 51.163 | 9.646 | 1.00 | 76.88 | N |
| ATOM | 12052 | C | ASN | B | 385 | 27.362 | 52.205 | 8.374 | 1.00 | 79.12 | C |
| ATOM | 12053 | O | ASN | B | 385 | 27.234 | 51.384 | 7.455 | 1.00 | 79.49 | O |
| ATOM | 12054 | OXT | ASN | B | 385 | 26.445 | 53.064 | 8.344 | 1.00 | 79.45 | O |
| ATOM | 12055 | N | SER | C | −2 | 53.798 | 42.195 | 56.398 | 1.00 | 54.33 | N |
| ATOM | 12057 | CA | SER | C | −2 | 54.991 | 42.203 | 57.314 | 1.00 | 54.26 | C |
| ATOM | 12059 | CB | SER | C | −2 | 54.496 | 42.894 | 58.620 | 1.00 | 59.81 | C |
| ATOM | 12062 | OG | SER | C | −2 | 53.217 | 43.571 | 58.434 | 1.00 | 50.05 | O |
| ATOM | 12064 | C | SER | C | −2 | 55.846 | 40.853 | 57.607 | 1.00 | 51.30 | C |
| ATOM | 12065 | O | SER | C | −2 | 56.969 | 40.946 | 58.008 | 1.00 | 52.44 | O |
| ATOM | 12068 | N | PHE | C | −1 | 55.366 | 39.626 | 57.416 | 1.00 | 49.50 | N |
| ATOM | 12070 | CA | PHE | C | −1 | 56.183 | 38.439 | 57.772 | 1.00 | 45.08 | C |
| ATOM | 12072 | CB | PHE | C | −1 | 55.401 | 37.604 | 58.777 | 1.00 | 45.10 | C |
| ATOM | 12075 | CG | PHE | C | −1 | 54.686 | 38.446 | 59.773 | 1.00 | 43.74 | C |
| ATOM | 12076 | CD1 | PHE | C | −1 | 53.717 | 39.362 | 59.347 | 1.00 | 39.81 | C |
| ATOM | 12078 | CE1 | PHE | C | −1 | 53.068 | 40.151 | 60.248 | 1.00 | 44.62 | C |
| ATOM | 12080 | CZ | PHE | C | −1 | 53.371 | 40.058 | 61.605 | 1.00 | 39.39 | C |
| ATOM | 12082 | CE2 | PHE | C | −1 | 54.336 | 39.149 | 62.031 | 1.00 | 45.60 | C |
| ATOM | 12084 | CD2 | PHE | C | −1 | 54.990 | 38.355 | 61.111 | 1.00 | 39.77 | C |
| ATOM | 12086 | C | PHE | C | −1 | 56.784 | 37.524 | 56.676 | 1.00 | 47.59 | C |
| ATOM | 12087 | O | PHE | C | −1 | 56.404 | 36.379 | 56.469 | 1.00 | 48.73 | O |
| ATOM | 12088 | N | VAL | C | 0 | 57.769 | 38.075 | 56.011 | 1.00 | 46.82 | N |
| ATOM | 12090 | CA | VAL | C | 0 | 58.543 | 37.462 | 54.968 | 1.00 | 42.56 | C |
| ATOM | 12092 | CB | VAL | C | 0 | 59.695 | 38.359 | 54.783 | 1.00 | 48.99 | C |
| ATOM | 12094 | CG1 | VAL | C | 0 | 59.357 | 39.397 | 53.764 | 1.00 | 51.10 | C |
| ATOM | 12098 | CG2 | VAL | C | 0 | 60.001 | 39.049 | 56.107 | 1.00 | 51.89 | C |
| ATOM | 12102 | C | VAL | C | 0 | 59.140 | 36.107 | 55.089 | 1.00 | 46.04 | C |
| ATOM | 12103 | O | VAL | C | 0 | 59.300 | 35.444 | 54.090 | 1.00 | 52.48 | O |
| ATOM | 12104 | N | GLU | C | 1 | 59.520 | 35.666 | 56.275 | 1.00 | 52.40 | N |
| ATOM | 12106 | CA | GLU | C | 1 | 60.053 | 34.310 | 56.418 | 1.00 | 48.09 | C |
| ATOM | 12108 | CD | GLU | C | 1 | 60.580 | 34.062 | 57.864 | 1.00 | 52.29 | C |
| ATOM | 12111 | CG | GLU | C | 1 | 61.892 | 33.262 | 57.999 | 1.00 | 57.28 | C |
| ATOM | 12114 | CD | GLU | C | 1 | 61.893 | 32.085 | 59.020 | 1.00 | 64.51 | C |
| ATOM | 12115 | OE1 | GLU | C | 1 | 61.939 | 32.255 | 60.267 | 1.00 | 69.01 | O |
| ATOM | 12116 | OE2 | GLU | C | 1 | 61.884 | 30.928 | 58.557 | 1.00 | 72.60 | O |
| ATOM | 12117 | C | GLU | C | 1 | 58.883 | 33.376 | 56.142 | 1.00 | 46.54 | C |
| ATOM | 12118 | O | GLU | C | 1 | 59.026 | 32.280 | 55.617 | 1.00 | 49.95 | O |
| ATOM | 12119 | N | MET | C | 2 | 57.696 | 33.827 | 56.493 | 1.00 | 49.04 | N |
| ATOM | 12121 | CA | MET | C | 2 | 56.532 | 32.945 | 56.564 | 1.00 | 49.74 | C |
| ATOM | 12123 | CB | MET | C | 2 | 55.626 | 33.430 | 57.694 | 1.00 | 48.27 | C |
| ATOM | 12126 | CG | MET | C | 2 | 56.231 | 33.224 | 59.038 | 1.00 | 45.70 | C |
| ATOM | 12129 | SD | MET | C | 2 | 55.044 | 33.016 | 60.326 | 1.00 | 51.30 | S |
| ATOM | 12130 | CE | MET | C | 2 | 54.993 | 34.710 | 60.885 | 1.00 | 55.15 | C |
| ATOM | 12134 | C | MET | C | 2 | 55.733 | 32.822 | 55.289 | 1.00 | 49.17 | C |
| ATOM | 12135 | O | MET | C | 2 | 55.047 | 31.832 | 55.051 | 1.00 | 53.00 | O |
| ATOM | 12136 | N | VAL | C | 3 | 55.829 | 33.826 | 54.453 | 1.00 | 48.17 | N |
| ATOM | 12138 | CA | VAL | C | 3 | 55.057 | 33.841 | 53.237 | 1.00 | 44.83 | C |
| ATOM | 12140 | CB | VAL | C | 3 | 55.288 | 35.185 | 52.611 | 1.00 | 44.77 | C |
| ATOM | 12142 | CG1 | VAL | C | 3 | 55.076 | 35.148 | 51.174 | 1.00 | 49.34 | C |
| ATOM | 12146 | CG2 | VAL | C | 3 | 54.379 | 36.181 | 53.266 | 1.00 | 47.06 | C |
| ATOM | 12150 | C | VAL | C | 3 | 55.391 | 32.689 | 52.296 | 1.00 | 42.19 | C |
| ATOM | 12151 | O | VAL | C | 3 | 56.538 | 32.338 | 52.136 | 1.00 | 44.06 | O |
| ATOM | 12152 | N | ASP | C | 4 | 54.375 | 32.108 | 51.668 | 1.00 | 45.69 | N |
| ATOM | 12154 | CA | ASP | C | 4 | 54.560 | 31.045 | 50.664 | 1.00 | 46.60 | C |
| ATOM | 12156 | CB | ASP | C | 4 | 55.500 | 31.467 | 49.499 | 1.00 | 49.07 | C |
| ATOM | 12159 | CG | ASP | C | 4 | 54.841 | 32.538 | 48.512 | 1.00 | 63.17 | C |
| ATOM | 12160 | OD1 | ASP | C | 4 | 55.334 | 32.743 | 47.356 | 1.00 | 68.98 | O |
| ATOM | 12161 | OD2 | ASP | C | 4 | 53.829 | 33.234 | 48.793 | 1.00 | 63.59 | O |
| ATOM | 12162 | C | ASP | C | 4 | 55.157 | 29.878 | 51.385 | 1.00 | 47.49 | C |
| ATOM | 12163 | O | ASP | C | 4 | 56.060 | 29.240 | 50.853 | 1.00 | 49.78 | O |
| ATOM | 12164 | N | ASN | C | 5 | 54.660 | 29.586 | 52.594 | 1.00 | 44.31 | N |
| ATOM | 12166 | CA | ASN | C | 5 | 55.266 | 28.538 | 53.398 | 1.00 | 36.68 | C |
| ATOM | 12168 | CB | ASN | C | 5 | 55.621 | 29.037 | 54.805 | 1.00 | 36.31 | C |
| ATOM | 12171 | CG | ASN | C | 5 | 54.437 | 29.178 | 55.738 | 1.00 | 40.22 | C |
| ATOM | 12172 | OD1 | ASN | C | 5 | 53.318 | 29.476 | 55.339 | 1.00 | 47.05 | O |
| ATOM | 12173 | ND2 | ASN | C | 5 | 54.694 | 28.960 | 57.017 | 1.00 | 41.17 | N |
| ATOM | 12176 | C | ASN | C | 5 | 54.452 | 27.285 | 53.431 | 1.00 | 41.07 | C |
| ATOM | 12177 | O | ASN | C | 5 | 54.800 | 26.347 | 54.177 | 1.00 | 38.77 | O |
| ATOM | 12178 | N | LEU | C | 6 | 53.389 | 27.284 | 52.615 | 1.00 | 40.12 | N |
| ATOM | 12180 | CA | LEU | C | 6 | 52.525 | 26.123 | 52.384 | 1.00 | 41.76 | C |
| ATOM | 12182 | CB | LEU | C | 6 | 51.101 | 26.491 | 52.744 | 1.00 | 42.43 | C |
| ATOM | 12185 | CG | LEU | C | 6 | 50.893 | 26.981 | 54.147 | 1.00 | 44.31 | C |
| ATOM | 12187 | CD1 | LEU | C | 6 | 49.566 | 27.697 | 54.187 | 1.00 | 45.43 | C |
| ATOM | 12191 | CD2 | LEU | C | 6 | 50.912 | 25.827 | 55.061 | 1.00 | 45.17 | C |
| ATOM | 12195 | C | LEU | C | 6 | 52.487 | 25.582 | 50.922 | 1.00 | 42.56 | C |
| ATOM | 12196 | O | LEU | C | 6 | 52.517 | 26.338 | 49.958 | 1.00 | 45.43 | O |
| ATOM | 12197 | N | ARG | C | 7 | 52.372 | 24.270 | 50.783 | 1.00 | 41.74 | N |
| ATOM | 12199 | CA | ARG | C | 7 | 52.291 | 23.598 | 49.484 | 1.00 | 46.94 | C |
| ATOM | 12201 | CB | ARG | C | 7 | 53.550 | 22.812 | 49.196 | 1.00 | 45.98 | C |
| ATOM | 12204 | CG | ARG | C | 7 | 54.688 | 23.617 | 48.729 | 1.00 | 52.89 | C |
| ATOM | 12207 | CD | ARG | C | 7 | 55.882 | 22.751 | 48.451 | 1.00 | 62.54 | C |
| ATOM | 12210 | NE | ARG | C | 7 | 56.264 | 21.967 | 49.632 | 1.00 | 72.96 | N |
| ATOM | 12212 | CZ | ARG | C | 7 | 57.069 | 20.891 | 49.617 | 1.00 | 78.99 | C |
| ATOM | 12213 | NH1 | ARG | C | 7 | 57.600 | 20.433 | 48.481 | 1.00 | 78.58 | N |
| ATOM | 12216 | NH2 | ARG | C | 7 | 57.346 | 20.266 | 50.751 | 1.00 | 78.67 | N |
| ATOM | 12219 | C | ARG | C | 7 | 51.138 | 22.617 | 49.483 | 1.00 | 44.60 | C |
| ATOM | 12220 | O | ARG | C | 7 | 50.627 | 22.304 | 50.529 | 1.00 | 50.99 | O |
| ATOM | 12221 | N | GLY | C | 8 | 50.752 | 22.119 | 48.312 | 1.00 | 50.43 | N |
| ATOM | 12223 | CA | GLY | C | 8 | 49.607 | 21.220 | 48.180 | 1.00 | 52.51 | C |
| ATOM | 12226 | C | GLY | C | 8 | 48.335 | 21.857 | 47.620 | 1.00 | 60.38 | C |
| ATOM | 12227 | O | GLY | C | 8 | 48.338 | 22.952 | 47.008 | 1.00 | 64.85 | O |
| ATOM | 12228 | N | LYS | C | 9 | 47.213 | 21.180 | 47.833 | 1.00 | 63.07 | N |
| ATOM | 12230 | CA | LYS | C | 9 | 45.965 | 21.642 | 47.255 | 1.00 | 64.03 | C |
| ATOM | 12232 | CB | LYS | C | 9 | 45.860 | 21.081 | 45.826 | 1.00 | 68.91 | C |
| ATOM | 12235 | CG | LYS | C | 9 | 46.401 | 19.663 | 45.630 | 1.00 | 71.59 | C |
| ATOM | 12238 | CD | LYS | C | 9 | 46.383 | 19.296 | 44.140 | 1.00 | 78.75 | C |
| ATOM | 12241 | CE | LYS | C | 9 | 46.052 | 17.793 | 43.893 | 1.00 | 82.33 | C |
| ATOM | 12244 | NZ | LYS | C | 9 | 46.104 | 17.357 | 42.454 | 1.00 | 80.13 | N |
| ATOM | 12248 | C | LYS | C | 9 | 44.696 | 21.313 | 48.068 | 1.00 | 63.52 | C |
| ATOM | 12249 | O | LYS | C | 9 | 44.734 | 20.619 | 49.076 | 1.00 | 66.42 | O |
| ATOM | 12250 | N | SER | C | 10 | 43.560 | 21.824 | 47.610 | 1.00 | 62.64 | N |
| ATOM | 12252 | CA | SER | C | 10 | 42.295 | 21.547 | 48.244 | 1.00 | 56.42 | C |
| ATOM | 12254 | CB | SER | C | 10 | 41.188 | 22.179 | 47.413 | 1.00 | 58.97 | C |
| ATOM | 12257 | OG | SER | C | 10 | 41.661 | 23.246 | 46.598 | 1.00 | 58.04 | O |
| ATOM | 12259 | C | SER | C | 10 | 42.115 | 20.024 | 48.308 | 1.00 | 59.45 | C |
| ATOM | 12260 | O | SER | C | 10 | 41.760 | 19.470 | 49.366 | 1.00 | 61.04 | O |
| ATOM | 12261 | N | GLY | C | 11 | 42.385 | 19.373 | 47.161 | 1.00 | 57.59 | N |
| ATOM | 12263 | CA | GLY | C | 11 | 42.230 | 17.938 | 46.946 | 1.00 | 52.60 | C |
| ATOM | 12266 | C | GLY | C | 11 | 42.837 | 17.013 | 47.974 | 1.00 | 50.69 | C |
| ATOM | 12267 | O | GLY | C | 11 | 42.155 | 16.131 | 48.456 | 1.00 | 47.76 | O |
| ATOM | 12268 | N | GLN | C | 12 | 44.111 | 17.214 | 48.304 | 1.00 | 51.44 | N |
| ATOM | 12270 | CA | GLN | C | 12 | 44.796 | 16.397 | 49.296 | 1.00 | 51.75 | C |
| ATOM | 12272 | CB | GLN | C | 12 | 45.904 | 15.638 | 48.615 | 1.00 | 51.49 | C |
| ATOM | 12275 | CG | GLN | C | 12 | 45.346 | 15.061 | 47.327 | 1.00 | 57.41 | C |
| ATOM | 12278 | CD | GLN | C | 12 | 46.330 | 14.266 | 46.496 | 1.00 | 62.60 | C |
| ATOM | 12279 | OE1 | GLN | C | 12 | 47.536 | 14.564 | 46.471 | 1.00 | 67.40 | O |
| ATOM | 12280 | NE2 | GLN | C | 12 | 45.816 | 13.255 | 45.798 | 1.00 | 64.74 | N |
| ATOM | 12283 | C | GLN | C | 12 | 45.284 | 17.244 | 50.466 | 1.00 | 51.33 | C |
| ATOM | 12284 | O | GLN | C | 12 | 45.770 | 16.759 | 51.487 | 1.00 | 53.08 | O |
| ATOM | 12285 | N | GLY | C | 13 | 45.147 | 18.538 | 50.320 | 1.00 | 50.28 | N |
| ATOM | 12287 | CA | GLY | C | 13 | 45.357 | 19.400 | 51.455 | 1.00 | 48.93 | C |
| ATOM | 12290 | C | GLY | C | 13 | 46.550 | 20.283 | 51.408 | 1.00 | 42.27 | C |
| ATOM | 12291 | O | GLY | C | 13 | 47.417 | 20.135 | 50.579 | 1.00 | 38.82 | O |
| ATOM | 12292 | N | TYR | C | 14 | 46.581 | 21.229 | 52.329 | 1.00 | 44.75 | N |
| ATOM | 12294 | CA | TYR | C | 14 | 47.763 | 22.054 | 52.493 | 1.00 | 42.35 | C |
| ATOM | 12296 | CB | TYR | C | 14 | 47.314 | 23.465 | 52.768 | 1.00 | 39.78 | C |
| ATOM | 12299 | CG | TYR | C | 14 | 46.558 | 23.964 | 51.576 | 1.00 | 40.40 | C |
| ATOM | 12300 | CD1 | TYR | C | 14 | 45.196 | 23.694 | 51.430 | 1.00 | 41.19 | C |
| ATOM | 12302 | CE1 | TYR | C | 14 | 44.482 | 24.133 | 50.330 | 1.00 | 37.98 | C |
| ATOM | 12304 | CZ | TYR | C | 14 | 45.138 | 24.850 | 49.355 | 1.00 | 51.75 | C |
| ATOM | 12305 | OH | TYR | C | 14 | 44.440 | 25.307 | 48.237 | 1.00 | 60.99 | O |
| ATOM | 12307 | CE2 | TYR | C | 14 | 46.508 | 25.122 | 49.486 | 1.00 | 48.63 | C |
| ATOM | 12309 | CD2 | TYR | C | 14 | 47.201 | 24.665 | 50.587 | 1.00 | 36.78 | C |
| ATOM | 12311 | C | TYR | C | 14 | 48.582 | 21.534 | 53.621 | 1.00 | 42.85 | C |
| ATOM | 12312 | O | TYR | C | 14 | 48.031 | 21.156 | 54.661 | 1.00 | 46.82 | O |
| ATOM | 12313 | N | TYR | C | 15 | 49.901 | 21.495 | 53.419 | 1.00 | 47.44 | N |
| ATOM | 12315 | CA | TYR | C | 15 | 50.864 | 21.109 | 54.484 | 1.00 | 40.77 | C |
| ATOM | 12317 | CB | TYR | C | 15 | 51.541 | 19.780 | 54.155 | 1.00 | 39.30 | C |
| ATOM | 12320 | CG | TYR | C | 15 | 52.293 | 19.646 | 52.807 | 1.00 | 35.74 | C |
| ATOM | 12321 | CD1 | TYR | C | 15 | 53.646 | 19.867 | 52.722 | 1.00 | 36.76 | C |
| ATOM | 12323 | CE1 | TYR | C | 15 | 54.335 | 19.731 | 51.545 | 1.00 | 33.20 | C |
| ATOM | 12325 | CZ | TYR | C | 15 | 53.703 | 19.360 | 50.429 | 1.00 | 37.91 | C |
| ATOM | 12326 | OH | TYR | C | 15 | 54.451 | 19.241 | 49.295 | 1.00 | 50.75 | O |
| ATOM | 12328 | CE2 | TYR | C | 15 | 52.355 | 19.111 | 50.433 | 1.00 | 39.45 | C |
| ATOM | 12330 | CD2 | TYR | C | 15 | 51.649 | 19.247 | 51.639 | 1.00 | 44.32 | C |
| ATOM | 12332 | C | TYR | C | 15 | 51.975 | 22.136 | 54.737 | 1.00 | 42.85 | C |
| ATOM | 12333 | O | TYR | C | 15 | 52.453 | 22.796 | 53.816 | 1.00 | 40.28 | O |
| ATOM | 12334 | N | VAL | C | 16 | 52.382 | 22.262 | 55.993 | 1.00 | 39.99 | N |
| ATOM | 12336 | CA | VAL | C | 16 | 53.542 | 23.061 | 56.317 | 1.00 | 40.96 | C |
| ATOM | 12338 | CB | VAL | C | 16 | 53.287 | 24.047 | 57.453 | 1.00 | 43.78 | C |
| ATOM | 12340 | CG1 | VAL | C | 16 | 53.114 | 23.309 | 58.754 | 1.00 | 40.96 | C |
| ATOM | 12344 | CG2 | VAL | C | 16 | 54.458 | 25.009 | 57.596 | 1.00 | 47.68 | C |
| ATOM | 12348 | C | VAL | C | 16 | 54.633 | 22.086 | 56.745 | 1.00 | 37.23 | C |
| ATOM | 12349 | O | VAL | C | 16 | 54.372 | 21.000 | 57.194 | 1.00 | 40.88 | O |
| ATOM | 12350 | N | GLU | C | 17 | 55.867 | 22.486 | 56.602 | 1.00 | 35.29 | N |
| ATOM | 12352 | CA | GLU | C | 17 | 56.982 | 21.642 | 56.919 | 1.00 | 35.79 | C |
| ATOM | 12354 | CB | GLU | C | 17 | 58.118 | 22.041 | 56.014 | 1.00 | 39.93 | C |
| ATOM | 12357 | CG | GLU | C | 17 | 59.452 | 21.517 | 56.451 | 1.00 | 47.15 | C |
| ATOM | 12360 | CD | GLU | C | 17 | 60.541 | 21.922 | 55.490 | 1.00 | 52.60 | C |
| ATOM | 12361 | OE1 | GLU | C | 17 | 60.212 | 22.301 | 54.325 | 1.00 | 57.45 | O |
| ATOM | 12362 | OE2 | GLU | C | 17 | 61.710 | 21.869 | 55.920 | 1.00 | 52.23 | O |
| ATOM | 12363 | C | GLU | C | 17 | 57.372 | 21.875 | 58.349 | 1.00 | 34.51 | C |
| ATOM | 12364 | O | GLU | C | 17 | 57.460 | 23.030 | 58.756 | 1.00 | 45.63 | O |
| ATOM | 12365 | N | MET | C | 18 | 57.592 | 20.818 | 59.130 | 1.00 | 33.60 | N |
| ATOM | 12367 | CA | MET | C | 18 | 57.970 | 20.988 | 60.539 | 1.00 | 39.59 | C |
| ATOM | 12369 | CB | MET | C | 18 | 56.772 | 20.810 | 61.479 | 1.00 | 40.20 | C |
| ATOM | 12372 | CG | MET | C | 18 | 55.481 | 21.510 | 61.062 | 1.00 | 40.31 | C |
| ATOM | 12375 | SD | MET | C | 18 | 54.035 | 21.028 | 62.062 | 1.00 | 49.53 | S |
| ATOM | 12376 | CE | MET | C | 18 | 54.480 | 21.689 | 63.652 | 1.00 | 40.70 | C |
| ATOM | 12380 | C | MET | C | 18 | 59.081 | 19.985 | 60.893 | 1.00 | 45.22 | C |
| ATOM | 12381 | O | MET | C | 18 | 59.490 | 19.197 | 60.024 | 1.00 | 48.94 | O |
| ATOM | 12382 | N | THR | C | 19 | 59.573 | 20.023 | 62.141 | 1.00 | 44.96 | N |
| ATOM | 12384 | CA | THR | C | 19 | 60.580 | 19.059 | 62.632 | 1.00 | 45.77 | C |
| ATOM | 12386 | CB | THR | C | 19 | 62.016 | 19.637 | 62.592 | 1.00 | 45.75 | C |
| ATOM | 12388 | OG1 | THR | C | 19 | 62.205 | 20.560 | 63.672 | 1.00 | 45.68 | O |
| ATOM | 12390 | CG2 | THR | C | 19 | 62.279 | 20.440 | 61.365 | 1.00 | 47.54 | C |
| ATOM | 12394 | C | THR | C | 19 | 60.360 | 18.645 | 64.085 | 1.00 | 46.35 | C |
| ATOM | 12395 | O | THR | C | 19 | 60.282 | 19.493 | 64.943 | 1.00 | 51.49 | O |
| ATOM | 12396 | N | VAL | C | 20 | 60.291 | 17.353 | 64.370 | 1.00 | 46.60 | N |
| ATOM | 12398 | CA | VAL | C | 20 | 60.207 | 16.880 | 65.747 | 1.00 | 46.13 | C |
| ATOM | 12400 | CB | VAL | C | 20 | 59.138 | 15.840 | 65.906 | 1.00 | 49.43 | C |
| ATOM | 12402 | CG1 | VAL | C | 20 | 57.730 | 16.382 | 65.578 | 1.00 | 55.74 | C |
| ATOM | 12406 | CG2 | VAL | C | 20 | 59.430 | 14.713 | 64.993 | 1.00 | 52.81 | C |
| ATOM | 12410 | C | VAL | C | 20 | 61.507 | 16.160 | 66.163 | 1.00 | 48.31 | C |
| ATOM | 12411 | O | VAL | C | 20 | 62.179 | 15.526 | 65.337 | 1.00 | 40.37 | O |
| ATOM | 12412 | N | GLY | C | 21 | 61.860 | 16.263 | 67.445 | 1.00 | 47.24 | N |
| ATOM | 12414 | CA | GLY | C | 21 | 62.935 | 15.466 | 68.008 | 1.00 | 44.07 | C |
| ATOM | 12417 | C | GLY | C | 21 | 64.315 | 16.030 | 67.964 | 1.00 | 48.42 | C |
| ATOM | 12418 | O | GLY | C | 21 | 64.512 | 17.180 | 67.577 | 1.00 | 54.75 | O |
| ATOM | 12419 | N | SER | C | 22 | 65.294 | 15.215 | 68.358 | 1.00 | 46.60 | N |
| ATOM | 12421 | CA | SER | C | 22 | 66.649 | 15.685 | 68.411 | 1.00 | 44.93 | C |
| ATOM | 12423 | CB | SER | C | 22 | 66.925 | 16.240 | 69.813 | 1.00 | 46.66 | C |
| ATOM | 12426 | OG | SER | C | 22 | 65.736 | 16.738 | 70.428 | 1.00 | 51.42 | O |
| ATOM | 12428 | C | SER | C | 22 | 67.639 | 14.580 | 68.113 | 1.00 | 47.58 | C |
| ATOM | 12429 | O | SER | C | 22 | 67.657 | 13.570 | 68.805 | 1.00 | 57.41 | O |
| ATOM | 12430 | N | PRO | C | 23 | 68.450 | 14.740 | 67.076 | 1.00 | 43.72 | N |
| ATOM | 12431 | CA | PRO | C | 23 | 68.345 | 15.844 | 66.127 | 1.00 | 46.60 | C |
| ATOM | 12433 | CB | PRO | C | 23 | 69.475 | 15.527 | 65.140 | 1.00 | 44.76 | C |
| ATOM | 12436 | CG | PRO | C | 23 | 69.592 | 14.109 | 65.254 | 1.00 | 41.34 | C |
| ATOM | 12439 | CD | PRO | C | 23 | 69.568 | 13.881 | 66.732 | 1.00 | 38.07 | C |
| ATOM | 12442 | C | PRO | C | 23 | 67.003 | 15.754 | 65.433 | 1.00 | 44.72 | C |
| ATOM | 12443 | O | PRO | C | 23 | 66.338 | 14.767 | 65.601 | 1.00 | 49.06 | O |
| ATOM | 12444 | N | PRO | C | 24 | 66.640 | 16.763 | 64.661 | 1.00 | 50.13 | N |
| ATOM | 12445 | CA | PRO | C | 24 | 65.314 | 16.882 | 64.016 | 1.00 | 45.48 | C |
| ATOM | 12447 | CB | PRO | C | 24 | 65.324 | 18.328 | 63.526 | 1.00 | 50.03 | C |
| ATOM | 12450 | CG | PRO | C | 24 | 66.494 | 19.014 | 64.223 | 1.00 | 50.43 | C |
| ATOM | 12453 | CD | PRO | C | 24 | 67.515 | 17.933 | 64.408 | 1.00 | 46.95 | C |
| ATOM | 12456 | C | PRO | C | 24 | 64.902 | 16.046 | 62.798 | 1.00 | 47.53 | C |
| ATOM | 12457 | O | PRO | C | 24 | 65.687 | 15.938 | 61.873 | 1.00 | 48.93 | O |
| ATOM | 12458 | N | GLN | C | 25 | 63.661 | 15.568 | 62.799 | 1.00 | 50.98 | N |
| ATOM | 12460 | CA | GLN | C | 25 | 63.096 | 14.893 | 61.663 | 1.00 | 49.33 | C |
| ATOM | 12462 | CB | GLN | C | 25 | 62.395 | 13.625 | 62.149 | 1.00 | 53.14 | C |
| ATOM | 12465 | CG | GLN | C | 25 | 63.395 | 12.605 | 62.669 | 1.00 | 48.07 | C |
| ATOM | 12468 | CD | GLN | C | 25 | 62.765 | 11.501 | 63.465 | 1.00 | 52.67 | C |
| ATOM | 12469 | OE1 | GLN | C | 25 | 61.814 | 10.862 | 63.017 | 1.00 | 63.47 | O |
| ATOM | 12470 | NE2 | GLN | C | 25 | 63.303 | 11.255 | 64.648 | 1.00 | 46.81 | N |
| ATOM | 12473 | C | GLN | C | 25 | 62.148 | 15.776 | 60.845 | 1.00 | 51.50 | C |
| ATOM | 12474 | O | GLN | C | 25 | 61.039 | 16.151 | 61.274 | 1.00 | 46.44 | O |
| ATOM | 12475 | N | THR | C | 26 | 62.595 | 16.092 | 59.643 | 1.00 | 49.81 | N |
| ATOM | 12477 | CA | THR | C | 26 | 61.754 | 16.827 | 58.751 | 1.00 | 51.29 | C |
| ATOM | 12479 | CB | THR | C | 26 | 62.462 | 17.098 | 57.429 | 1.00 | 53.77 | C |
| ATOM | 12481 | OG1 | THR | C | 26 | 63.759 | 17.659 | 57.673 | 1.00 | 53.98 | O |
| ATOM | 12483 | CG2 | THR | C | 26 | 61.709 | 18.172 | 56.629 | 1.00 | 52.68 | C |
| ATOM | 12487 | C | THR | C | 26 | 60.555 | 15.969 | 58.478 | 1.00 | 51.30 | C |
| ATOM | 12488 | O | THR | C | 26 | 60.666 | 14.771 | 58.226 | 1.00 | 51.90 | O |
| ATOM | 12489 | N | LEU | C | 27 | 59.396 | 16.594 | 58.530 | 1.00 | 50.70 | N |
| ATOM | 12491 | CA | LEU | C | 27 | 58.174 | 15.933 | 58.155 | 1.00 | 43.30 | C |
| ATOM | 12493 | CB | LEU | C | 27 | 57.478 | 15.409 | 59.378 | 1.00 | 44.64 | C |
| ATOM | 12496 | CG | LEU | C | 27 | 58.218 | 14.449 | 60.281 | 1.00 | 45.47 | C |
| ATOM | 12498 | CD1 | LEU | C | 27 | 57.624 | 14.540 | 61.675 | 1.00 | 45.75 | C |
| ATOM | 12502 | CD2 | LEU | C | 27 | 58.122 | 13.034 | 59.686 | 1.00 | 44.30 | C |
| ATOM | 12506 | C | LEU | C | 27 | 57.323 | 17.006 | 57.577 | 1.00 | 39.69 | C |
| ATOM | 12507 | O | LEU | C | 27 | 57.495 | 18.143 | 57.925 | 1.00 | 43.38 | O |
| ATOM | 12508 | N | ASN | C | 28 | 56.412 | 16.652 | 56.695 | 1.00 | 42.74 | N |
| ATOM | 12510 | CA | ASN | C | 28 | 55.433 | 17.586 | 56.185 | 1.00 | 41.37 | C |
| ATOM | 12512 | CB | ASN | C | 28 | 55.194 | 17.347 | 54.703 | 1.00 | 42.25 | C |
| ATOM | 12515 | CG | ASN | C | 28 | 56.215 | 18.014 | 53.828 | 1.00 | 41.51 | C |
| ATOM | 12516 | OD1 | ASN | C | 28 | 56.417 | 17.614 | 52.679 | 1.00 | 48.06 | O |
| ATOM | 12517 | ND2 | ASN | C | 28 | 56.870 | 19.034 | 54.355 | 1.00 | 48.36 | N |
| ATOM | 12520 | C | ASN | C | 28 | 54.144 | 17.326 | 56.948 | 1.00 | 42.07 | C |
| ATOM | 12521 | O | ASN | C | 28 | 53.796 | 16.175 | 57.196 | 1.00 | 42.00 | O |
| ATOM | 12522 | N | ILE | C | 29 | 53.429 | 18.388 | 57.303 | 1.00 | 41.25 | N |
| ATOM | 12524 | CA | ILE | C | 29 | 52.254 | 18.282 | 58.144 | 1.00 | 38.11 | C |
| ATOM | 12526 | CB | ILE | C | 29 | 52.581 | 18.909 | 59.452 | 1.00 | 37.30 | C |
| ATOM | 12528 | CG1 | ILE | C | 29 | 53.888 | 18.345 | 60.002 | 1.00 | 37.82 | C |
| ATOM | 12531 | CD1 | ILE | C | 29 | 53.764 | 16.947 | 60.383 | 1.00 | 37.89 | C |
| ATOM | 12535 | CG2 | ILE | C | 29 | 51.428 | 18.758 | 60.382 | 1.00 | 37.26 | C |
| ATOM | 12539 | C | ILE | C | 29 | 51.111 | 19.100 | 57.605 | 1.00 | 41.88 | C |
| ATOM | 12540 | O | ILE | C | 29 | 51.317 | 20.272 | 57.335 | 1.00 | 44.35 | O |
| ATOM | 12541 | N | LEU | C | 30 | 49.924 | 18.485 | 57.471 | 1.00 | 41.50 | N |
| ATOM | 12543 | CA | LEU | C | 30 | 48.669 | 19.159 | 57.062 | 1.00 | 40.03 | C |
| ATOM | 12545 | CB | LEU | C | 30 | 47.609 | 18.121 | 56.699 | 1.00 | 36.38 | C |
| ATOM | 12548 | CG | LEU | C | 30 | 46.175 | 18.515 | 56.462 | 1.00 | 43.90 | C |
| ATOM | 12550 | CD1 | LEU | C | 30 | 45.650 | 17.851 | 55.272 | 1.00 | 48.94 | C |
| ATOM | 12554 | CD2 | LEU | C | 30 | 45.293 | 18.120 | 57.618 | 1.00 | 49.66 | C |
| ATOM | 12558 | C | LEU | C | 30 | 48.071 | 20.130 | 58.085 | 1.00 | 41.43 | C |
| ATOM | 12559 | O | LEU | C | 30 | 47.961 | 19.834 | 59.261 | 1.00 | 44.99 | O |
| ATOM | 12560 | N | VAL | C | 31 | 47.656 | 21.300 | 57.607 | 1.00 | 44.35 | N |
| ATOM | 12562 | CA | VAL | C | 31 | 47.080 | 22.312 | 58.460 | 1.00 | 40.92 | C |
| ATOM | 12564 | CB | VAL | C | 31 | 47.300 | 23.638 | 57.892 | 1.00 | 37.95 | C |
| ATOM | 12566 | CG1 | VAL | C | 31 | 46.317 | 24.595 | 58.524 | 1.00 | 41.29 | C |
| ATOM | 12570 | CG2 | VAL | C | 31 | 48.718 | 24.068 | 58.132 | 1.00 | 41.26 | C |
| ATOM | 12574 | C | VAL | C | 31 | 45.574 | 22.155 | 58.515 | 1.00 | 45.40 | C |
| ATOM | 12575 | O | VAL | C | 31 | 44.885 | 22.495 | 57.555 | 1.00 | 46.26 | O |
| ATOM | 12576 | N | ASP | C | 32 | 45.082 | 21.674 | 59.658 | 1.00 | 44.37 | N |
| ATOM | 12578 | CA | ASP | C | 32 | 43.693 | 21.316 | 59.849 | 1.00 | 42.08 | C |
| ATOM | 12580 | CB | ASP | C | 32 | 43.648 | 19.799 | 60.070 | 1.00 | 48.54 | C |
| ATOM | 12583 | CG | ASP | C | 32 | 42.270 | 19.279 | 60.373 | 1.00 | 55.02 | C |
| ATOM | 12584 | OD1 | ASP | C | 32 | 41.331 | 19.695 | 59.667 | 1.00 | 57.45 | O |
| ATOM | 12585 | OD2 | ASP | C | 32 | 42.035 | 18.443 | 61.298 | 1.00 | 54.56 | O |
| ATOM | 12586 | C | ASP | C | 32 | 42.996 | 22.030 | 60.996 | 1.00 | 42.96 | C |
| ATOM | 12587 | O | ASP | C | 32 | 43.171 | 21.653 | 62.148 | 1.00 | 38.86 | O |
| ATOM | 12588 | N | THR | C | 33 | 42.205 | 23.072 | 60.670 | 1.00 | 42.49 | N |
| ATOM | 12590 | CA | THR | C | 33 | 41.382 | 23.757 | 61.655 | 1.00 | 34.59 | C |
| ATOM | 12592 | CB | THR | C | 33 | 41.102 | 25.275 | 61.297 | 1.00 | 40.43 | C |
| ATOM | 12594 | OG1 | THR | C | 33 | 40.271 | 25.418 | 60.128 | 1.00 | 36.06 | O |
| ATOM | 12596 | CG2 | THR | C | 33 | 42.332 | 26.064 | 60.943 | 1.00 | 38.42 | C |
| ATOM | 12600 | C | THR | C | 33 | 40.025 | 23.151 | 61.903 | 1.00 | 37.84 | C |
| ATOM | 12601 | O | THR | C | 33 | 39.181 | 23.773 | 62.562 | 1.00 | 42.84 | O |
| ATOM | 12602 | N | GLY | C | 34 | 39.792 | 21.985 | 61.347 | 1.00 | 39.71 | N |
| ATOM | 12604 | CA | GLY | C | 34 | 38.621 | 21.204 | 61.713 | 1.00 | 45.14 | C |
| ATOM | 12607 | C | GLY | C | 34 | 38.955 | 20.018 | 62.634 | 1.00 | 44.20 | C |
| ATOM | 12608 | O | GLY | C | 34 | 38.190 | 19.062 | 62.710 | 1.00 | 44.56 | O |
| ATOM | 12609 | N | SER | C | 35 | 40.102 | 20.086 | 63.318 | 1.00 | 39.94 | N |
| ATOM | 12611 | CA | SER | C | 35 | 40.490 | 19.124 | 64.357 | 1.00 | 36.97 | C |
| ATOM | 12613 | CB | SER | C | 35 | 41.195 | 17.909 | 63.768 | 1.00 | 41.17 | C |
| ATOM | 12616 | OG | SER | C | 35 | 42.575 | 18.109 | 63.530 | 1.00 | 39.49 | O |
| ATOM | 12618 | C | SER | C | 35 | 41.423 | 19.799 | 65.385 | 1.00 | 41.65 | C |
| ATOM | 12619 | O | SER | C | 35 | 41.747 | 20.986 | 65.259 | 1.00 | 38.82 | O |
| ATOM | 12620 | N | SER | C | 36 | 41.881 | 19.044 | 66.384 | 1.00 | 41.66 | N |
| ATOM | 12622 | CA | SER | C | 36 | 42.683 | 19.621 | 67.476 | 1.00 | 36.97 | C |
| ATOM | 12624 | CB | SER | C | 36 | 41.755 | 19.879 | 68.673 | 1.00 | 39.49 | C |
| ATOM | 12627 | OG | SER | C | 36 | 40.776 | 20.894 | 68.393 | 1.00 | 34.74 | O |
| ATOM | 12629 | C | SER | C | 36 | 43.908 | 18.814 | 67.975 | 1.00 | 40.18 | C |
| ATOM | 12630 | O | SER | C | 36 | 44.478 | 19.169 | 69.000 | 1.00 | 46.46 | O |
| ATOM | 12631 | N | ASN | C | 37 | 44.322 | 17.742 | 67.298 | 1.00 | 34.55 | N |
| ATOM | 12633 | CA | ASN | C | 37 | 45.511 | 17.024 | 67.723 | 1.00 | 36.25 | C |
| ATOM | 12635 | CB | ASN | C | 37 | 45.259 | 15.525 | 67.766 | 1.00 | 38.28 | C |
| ATOM | 12638 | CG | ASN | C | 37 | 44.338 | 15.132 | 68.855 | 1.00 | 39.72 | C |
| ATOM | 12639 | OD1 | ASN | C | 37 | 43.120 | 15.086 | 68.681 | 1.00 | 38.23 | O |
| ATOM | 12640 | ND2 | ASN | C | 37 | 44.911 | 14.815 | 69.992 | 1.00 | 37.80 | N |
| ATOM | 12643 | C | ASN | C | 37 | 46.674 | 17.175 | 66.771 | 1.00 | 37.54 | C |
| ATOM | 12644 | O | ASN | C | 37 | 46.475 | 17.306 | 65.578 | 1.00 | 34.71 | O |
| ATOM | 12645 | N | PHE | C | 38 | 47.888 | 17.150 | 67.322 | 1.00 | 35.88 | N |
| ATOM | 12647 | CA | PHE | C | 38 | 49.090 | 17.051 | 66.525 | 1.00 | 33.22 | C |
| ATOM | 12649 | CB | PHE | C | 38 | 50.203 | 17.902 | 67.126 | 1.00 | 31.39 | C |
| ATOM | 12652 | CG | PHE | C | 38 | 51.541 | 17.943 | 66.303 | 1.00 | 37.60 | C |
| ATOM | 12653 | CD1 | PHE | C | 38 | 52.748 | 18.100 | 66.931 | 1.00 | 37.64 | C |
| ATOM | 12655 | CE1 | PHE | C | 38 | 53.864 | 18.133 | 66.256 | 1.00 | 42.48 | C |
| ATOM | 12657 | CZ | PHE | C | 38 | 53.869 | 18.028 | 64.938 | 1.00 | 41.33 | C |
| ATOM | 12659 | CE2 | PHE | C | 38 | 52.742 | 17.885 | 64.307 | 1.00 | 43.17 | C |
| ATOM | 12661 | CD2 | PHE | C | 38 | 51.573 | 17.844 | 64.979 | 1.00 | 38.57 | C |
| ATOM | 12663 | C | PHE | C | 38 | 49.361 | 15.548 | 66.531 | 1.00 | 31.89 | C |
| ATOM | 12664 | O | PHE | C | 38 | 49.328 | 14.912 | 67.596 | 1.00 | 30.08 | O |
| ATOM | 12665 | N | ALA | C | 39 | 49.584 | 14.984 | 65.337 | 1.00 | 35.67 | N |
| ATOM | 12667 | CA | ALA | C | 39 | 49.777 | 13.542 | 65.135 | 1.00 | 35.28 | C |
| ATOM | 12669 | CB | ALA | C | 39 | 48.450 | 12.917 | 65.095 | 1.00 | 37.14 | C |
| ATOM | 12673 | C | ALA | C | 39 | 50.557 | 13.217 | 63.833 | 1.00 | 41.38 | C |
| ATOM | 12674 | O | ALA | C | 39 | 50.252 | 13.780 | 62.781 | 1.00 | 47.14 | O |
| ATOM | 12675 | N | VAL | C | 40 | 51.556 | 12.330 | 63.900 | 1.00 | 44.87 | N |
| ATOM | 12677 | CA | VAL | C | 40 | 52.402 | 11.999 | 62.744 | 1.00 | 46.80 | C |
| ATOM | 12679 | CB | VAL | C | 40 | 53.865 | 12.384 | 62.950 | 1.00 | 51.51 | C |
| ATOM | 12681 | CG1 | VAL | C | 40 | 54.002 | 13.840 | 63.101 | 1.00 | 54.58 | C |
| ATOM | 12685 | CG2 | VAL | C | 40 | 54.444 | 11.672 | 64.169 | 1.00 | 57.23 | C |
| ATOM | 12689 | C | VAL | C | 40 | 52.504 | 10.524 | 62.628 | 1.00 | 44.81 | C |
| ATOM | 12690 | O | VAL | C | 40 | 52.333 | 9.858 | 63.627 | 1.00 | 47.35 | O |
| ATOM | 12691 | N | GLY | C | 41 | 52.796 | 10.017 | 61.429 | 1.00 | 42.10 | N |
| ATOM | 12693 | CA | GLY | C | 41 | 53.024 | 8.600 | 61.217 | 1.00 | 36.51 | C |
| ATOM | 12696 | C | GLY | C | 41 | 54.331 | 8.220 | 61.900 | 1.00 | 45.89 | C |
| ATOM | 12697 | O | GLY | C | 41 | 55.312 | 8.941 | 61.803 | 1.00 | 41.98 | O |
| ATOM | 12698 | N | ALA | C | 42 | 54.368 | 7.083 | 62.591 | 1.00 | 49.43 | N |
| ATOM | 12700 | CA | ALA | C | 42 | 55.588 | 6.687 | 63.291 | 1.00 | 46.63 | C |
| ATOM | 12702 | CB | ALA | C | 42 | 55.434 | 6.999 | 64.733 | 1.00 | 44.75 | C |
| ATOM | 12706 | C | ALA | C | 42 | 55.868 | 5.196 | 63.096 | 1.00 | 50.91 | C |
| ATOM | 12707 | O | ALA | C | 42 | 56.415 | 4.512 | 63.968 | 1.00 | 56.15 | O |
| ATOM | 12708 | N | ALA | C | 43 | 55.494 | 4.714 | 61.923 | 1.00 | 51.91 | N |
| ATOM | 12710 | CA | ALA | C | 43 | 55.530 | 3.302 | 61.647 | 1.00 | 55.01 | C |
| ATOM | 12712 | CB | ALA | C | 43 | 54.578 | 2.588 | 62.522 | 1.00 | 56.93 | C |
| ATOM | 12716 | C | ALA | C | 43 | 55.118 | 3.057 | 60.232 | 1.00 | 59.43 | C |
| ATOM | 12717 | O | ALA | C | 43 | 54.070 | 3.538 | 59.772 | 1.00 | 58.38 | O |
| ATOM | 12718 | N | PRO | C | 44 | 55.971 | 2.315 | 59.555 | 1.00 | 59.53 | N |
| ATOM | 12719 | CA | PRO | C | 44 | 55.738 | 1.800 | 58.207 | 1.00 | 57.91 | C |
| ATOM | 12721 | CB | PRO | C | 44 | 56.185 | 0.392 | 58.373 | 1.00 | 58.24 | C |
| ATOM | 12724 | CG | PRO | C | 44 | 57.450 | 0.628 | 59.193 | 1.00 | 60.25 | C |
| ATOM | 12727 | CD | PRO | C | 44 | 57.296 | 1.930 | 60.015 | 1.00 | 59.83 | C |
| ATOM | 12730 | C | PRO | C | 44 | 54.345 | 1.869 | 57.679 | 1.00 | 51.08 | C |
| ATOM | 12731 | O | PRO | C | 44 | 53.427 | 1.440 | 58.331 | 1.00 | 42.11 | O |
| ATOM | 12732 | N | HIS | C | 45 | 54.228 | 2.439 | 56.483 | 1.00 | 51.18 | N |
| ATOM | 12734 | CA | HIS | C | 45 | 52.943 | 2.516 | 55.767 | 1.00 | 51.14 | C |
| ATOM | 12736 | CB | HIS | C | 45 | 51.987 | 3.544 | 56.361 | 1.00 | 49.59 | C |
| ATOM | 12739 | CG | HIS | C | 45 | 50.664 | 3.623 | 55.641 | 1.00 | 55.41 | C |
| ATOM | 12740 | ND1 | HIS | C | 45 | 50.552 | 4.007 | 54.318 | 1.00 | 50.15 | N |
| ATOM | 12742 | CE1 | HIS | C | 45 | 49.280 | 3.983 | 53.961 | 1.00 | 48.42 | C |
| ATOM | 12744 | NE2 | HIS | C | 45 | 48.563 | 3.582 | 54.993 | 1.00 | 48.32 | N |
| ATOM | 12746 | CD2 | HIS | C | 45 | 49.401 | 3.351 | 56.058 | 1.00 | 50.46 | C |
| ATOM | 12748 | C | HIS | C | 45 | 53.171 | 2.803 | 54.287 | 1.00 | 51.98 | C |
| ATOM | 12749 | O | HIS | C | 45 | 53.986 | 3.610 | 53.912 | 1.00 | 58.52 | O |
| ATOM | 12750 | N | PRO | C | 46 | 52.430 | 2.123 | 53.444 | 1.00 | 59.22 | N |
| ATOM | 12751 | CA | PRO | C | 46 | 52.535 | 2.244 | 51.977 | 1.00 | 58.93 | C |
| ATOM | 12753 | CB | PRO | C | 46 | 51.381 | 1.382 | 51.492 | 1.00 | 60.36 | C |
| ATOM | 12756 | CG | PRO | C | 46 | 51.169 | 0.420 | 52.553 | 1.00 | 62.16 | C |
| ATOM | 12759 | CD | PRO | C | 46 | 51.412 | 1.141 | 53.851 | 1.00 | 58.40 | C |
| ATOM | 12762 | C | PRO | C | 46 | 52.263 | 3.601 | 51.375 | 1.00 | 60.75 | C |
| ATOM | 12763 | O | PRO | C | 46 | 52.429 | 3.742 | 50.152 | 1.00 | 66.57 | O |
| ATOM | 12764 | N | PHE | C | 47 | 51.820 | 4.582 | 52.146 | 1.00 | 53.51 | N |
| ATOM | 12766 | CA | PHE | C | 47 | 51.687 | 5.885 | 51.534 | 1.00 | 54.37 | C |
| ATOM | 12768 | CB | PHE | C | 47 | 50.227 | 6.362 | 51.593 | 1.00 | 55.83 | C |
| ATOM | 12771 | CG | PHE | C | 47 | 49.248 | 5.477 | 50.798 | 1.00 | 56.85 | C |
| ATOM | 12772 | CD1 | PHE | C | 47 | 47.889 | 5.498 | 51.069 | 1.00 | 53.21 | C |
| ATOM | 12774 | CE1 | PHE | C | 47 | 47.022 | 4.712 | 50.358 | 1.00 | 58.68 | C |
| ATOM | 12776 | CZ | PHE | C | 47 | 47.494 | 3.872 | 49.346 | 1.00 | 53.11 | C |
| ATOM | 12778 | CE2 | PHE | C | 47 | 48.802 | 3.834 | 49.068 | 1.00 | 53.10 | C |
| ATOM | 12780 | CD2 | PHE | C | 47 | 49.693 | 4.630 | 49.784 | 1.00 | 59.43 | C |
| ATOM | 12782 | C | PHE | C | 47 | 52.728 | 6.896 | 52.106 | 1.00 | 53.35 | C |
| ATOM | 12783 | O | PHE | C | 47 | 52.739 | 8.075 | 51.708 | 1.00 | 47.38 | O |
| ATOM | 12784 | N | LEU | C | 48 | 53.602 | 6.407 | 52.998 | 1.00 | 47.46 | N |
| ATOM | 12786 | CA | LEU | C | 48 | 54.627 | 7.217 | 53.677 | 1.00 | 48.00 | C |
| ATOM | 12788 | CB | LEU | C | 48 | 54.585 | 6.868 | 55.163 | 1.00 | 44.63 | C |
| ATOM | 12791 | CG | LEU | C | 48 | 53.203 | 7.093 | 55.770 | 1.00 | 36.89 | C |
| ATOM | 12793 | CD1 | LEU | C | 48 | 53.226 | 7.095 | 57.269 | 1.00 | 32.35 | C |
| ATOM | 12797 | CD2 | LEU | C | 48 | 52.716 | 8.414 | 55.326 | 1.00 | 43.78 | C |
| ATOM | 12801 | C | LEU | C | 48 | 56.055 | 7.010 | 53.206 | 1.00 | 49.23 | C |
| ATOM | 12802 | O | LEU | C | 48 | 56.512 | 5.888 | 53.245 | 1.00 | 51.31 | O |
| ATOM | 12803 | N | HIS | C | 49 | 56.741 | 8.082 | 52.772 | 1.00 | 54.74 | N |
| ATOM | 12805 | CA | HIS | C | 49 | 58.138 | 8.036 | 52.290 | 1.00 | 55.16 | C |
| ATOM | 12807 | CB | HIS | C | 49 | 58.563 | 9.276 | 51.513 | 1.00 | 62.74 | C |
| ATOM | 12810 | CG | HIS | C | 49 | 57.851 | 9.479 | 50.216 | 1.00 | 80.32 | C |
| ATOM | 12811 | ND1 | HIS | C | 49 | 57.953 | 8.596 | 49.157 | 1.00 | 90.23 | N |
| ATOM | 12813 | CE1 | HIS | C | 49 | 57.221 | 9.041 | 48.148 | 1.00 | 90.85 | C |
| ATOM | 12815 | NE2 | HIS | C | 49 | 56.653 | 10.180 | 48.512 | 1.00 | 88.40 | N |
| ATOM | 12817 | CD2 | HIS | C | 49 | 57.032 | 10.477 | 49.799 | 1.00 | 85.66 | C |
| ATOM | 12819 | C | HIS | C | 49 | 58.985 | 8.110 | 53.491 | 1.00 | 52.56 | C |
| ATOM | 12820 | O | HIS | C | 49 | 60.083 | 7.550 | 53.529 | 1.00 | 57.27 | O |
| ATOM | 12821 | N | ARG | C | 50 | 58.495 | 8.859 | 54.472 | 1.00 | 46.86 | N |
| ATOM | 12823 | CA | ARG | C | 50 | 59.152 | 8.928 | 55.749 | 1.00 | 44.15 | C |
| ATOM | 12825 | CB | ARG | C | 50 | 60.123 | 10.094 | 55.776 | 1.00 | 47.15 | C |
| ATOM | 12828 | CG | ARG | C | 50 | 59.543 | 11.362 | 55.346 | 1.00 | 48.57 | C |
| ATOM | 12831 | CD | ARG | C | 50 | 60.498 | 12.504 | 55.358 | 1.00 | 47.15 | C |
| ATOM | 12834 | NE | ARG | C | 50 | 59.733 | 13.667 | 54.935 | 1.00 | 57.92 | N |
| ATOM | 12836 | CZ | ARG | C | 50 | 60.210 | 14.894 | 54.724 | 1.00 | 57.78 | C |
| ATOM | 12837 | NH1 | ARG | C | 50 | 61.491 | 15.192 | 54.882 | 1.00 | 51.00 | N |
| ATOM | 12840 | NH2 | ARG | C | 50 | 59.366 | 15.835 | 54.342 | 1.00 | 59.97 | N |
| ATOM | 12843 | C | ARG | C | 50 | 58.192 | 9.000 | 56.918 | 1.00 | 40.60 | C |
| ATOM | 12844 | O | ARG | C | 50 | 56.992 | 9.149 | 56.762 | 1.00 | 38.05 | O |
| ATOM | 12845 | N | TYR | C | 51 | 58.746 | 8.873 | 58.107 | 1.00 | 43.42 | N |
| ATOM | 12847 | CA | TYR | C | 51 | 57.961 | 8.988 | 59.304 | 1.00 | 46.93 | C |
| ATOM | 12849 | CB | TYR | C | 51 | 57.068 | 7.774 | 59.414 | 1.00 | 48.86 | C |
| ATOM | 12852 | CG | TYR | C | 51 | 57.838 | 6.503 | 59.490 | 1.00 | 57.25 | C |
| ATOM | 12853 | CD1 | TYR | C | 51 | 58.167 | 5.944 | 60.706 | 1.00 | 59.52 | C |
| ATOM | 12855 | CE1 | TYR | C | 51 | 58.876 | 4.753 | 60.768 | 1.00 | 64.00 | C |
| ATOM | 12857 | CZ | TYR | C | 51 | 59.260 | 4.118 | 59.593 | 1.00 | 65.22 | C |
| ATOM | 12858 | OH | TYR | C | 51 | 59.966 | 2.928 | 59.626 | 1.00 | 68.18 | O |
| ATOM | 12860 | CE2 | TYR | C | 51 | 58.939 | 4.662 | 58.380 | 1.00 | 61.62 | C |
| ATOM | 12862 | CD2 | TYR | C | 51 | 58.236 | 5.844 | 58.331 | 1.00 | 62.51 | C |
| ATOM | 12864 | C | TYR | C | 51 | 58.822 | 9.143 | 60.563 | 1.00 | 40.91 | C |
| ATOM | 12865 | O | TYR | C | 51 | 59.999 | 8.948 | 60.522 | 1.00 | 42.94 | O |
| ATOM | 12866 | N | TYR | C | 52 | 58.190 | 9.503 | 61.674 | 1.00 | 40.91 | N |
| ATOM | 12868 | CA | TYR | C | 52 | 58.832 | 9.726 | 62.976 | 1.00 | 40.27 | C |
| ATOM | 12870 | CB | TYR | C | 52 | 57.747 | 10.233 | 63.886 | 1.00 | 41.76 | C |
| ATOM | 12873 | CG | TYR | C | 52 | 58.122 | 10.764 | 65.232 | 1.00 | 41.11 | C |
| ATOM | 12874 | CD1 | TYR | C | 52 | 59.174 | 11.588 | 65.399 | 1.00 | 47.30 | C |
| ATOM | 12876 | CE1 | TYR | C | 52 | 59.492 | 12.084 | 66.637 | 1.00 | 50.41 | C |
| ATOM | 12878 | CZ | TYR | C | 52 | 58.750 | 11.768 | 67.719 | 1.00 | 49.29 | C |
| ATOM | 12879 | OH | TYR | C | 52 | 59.121 | 12.304 | 68.943 | 1.00 | 53.62 | O |
| ATOM | 12881 | CE2 | TYR | C | 52 | 57.685 | 10.943 | 67.579 | 1.00 | 46.11 | C |
| ATOM | 12883 | CD2 | TYR | C | 52 | 57.377 | 10.449 | 66.340 | 1.00 | 46.35 | C |
| ATOM | 12885 | C | TYR | C | 52 | 59.394 | 8.525 | 63.684 | 1.00 | 41.56 | C |
| ATOM | 12886 | O | TYR | C | 52 | 58.631 | 7.656 | 64.087 | 1.00 | 42.35 | O |
| ATOM | 12887 | N | GLN | C | 53 | 60.711 | 8.500 | 63.860 | 1.00 | 44.45 | N |
| ATOM | 12889 | CA | GLN | C | 53 | 61.404 | 7.421 | 64.534 | 1.00 | 41.54 | C |
| ATOM | 12891 | CB | GLN | C | 53 | 62.681 | 7.086 | 63.790 | 1.00 | 44.38 | C |
| ATOM | 12894 | CG | GLN | C | 53 | 62.440 | 6.932 | 62.289 | 1.00 | 51.42 | C |
| ATOM | 12897 | CD | GLN | C | 53 | 63.227 | 5.794 | 61.672 | 1.00 | 59.82 | C |
| ATOM | 12898 | OE1 | GLN | C | 53 | 63.842 | 5.956 | 60.617 | 1.00 | 61.98 | O |
| ATOM | 12899 | NE2 | GLN | C | 53 | 63.204 | 4.638 | 62.321 | 1.00 | 67.41 | N |
| ATOM | 12902 | C | GLN | C | 53 | 61.698 | 7.905 | 65.919 | 1.00 | 39.42 | C |
| ATOM | 12903 | O | GLN | C | 53 | 62.595 | 8.716 | 66.122 | 1.00 | 53.39 | O |
| ATOM | 12904 | N | ARG | C | 54 | 60.926 | 7.410 | 66.862 | 1.00 | 38.63 | N |
| ATOM | 12906 | CA | ARG | C | 54 | 61.004 | 7.780 | 68.276 | 1.00 | 42.43 | C |
| ATOM | 12908 | CB | ARG | C | 54 | 59.828 | 7.175 | 69.000 | 1.00 | 39.75 | C |
| ATOM | 12911 | CG | ARG | C | 54 | 58.574 | 8.077 | 68.924 | 1.00 | 40.81 | C |
| ATOM | 12914 | CD | ARG | C | 54 | 57.389 | 7.431 | 69.538 | 1.00 | 39.95 | C |
| ATOM | 12917 | NE | ARG | C | 54 | 57.137 | 6.168 | 68.844 | 1.00 | 40.64 | N |
| ATOM | 12919 | CZ | ARG | C | 54 | 56.124 | 5.372 | 69.092 | 1.00 | 45.54 | C |
| ATOM | 12920 | NH1 | ARG | C | 54 | 55.230 | 5.686 | 70.027 | 1.00 | 47.14 | N |
| ATOM | 12923 | NH2 | ARG | C | 54 | 56.000 | 4.257 | 68.396 | 1.00 | 48.98 | N |
| ATOM | 12926 | C | ARG | C | 54 | 62.219 | 7.302 | 69.005 | 1.00 | 47.24 | C |
| ATOM | 12927 | O | ARG | C | 54 | 62.621 | 7.870 | 70.013 | 1.00 | 49.66 | O |
| ATOM | 12928 | N | GLN | C | 55 | 62.795 | 6.236 | 68.482 | 1.00 | 55.23 | N |
| ATOM | 12930 | CA | GLN | C | 55 | 63.960 | 5.624 | 69.061 | 1.00 | 55.09 | C |
| ATOM | 12932 | CB | GLN | C | 55 | 64.162 | 4.277 | 68.388 | 1.00 | 60.40 | C |
| ATOM | 12935 | CG | GLN | C | 55 | 64.836 | 4.423 | 67.025 | 1.00 | 62.46 | C |
| ATOM | 12938 | CD | GLN | C | 55 | 63.988 | 4.048 | 65.850 | 1.00 | 61.26 | C |
| ATOM | 12939 | OE1 | GLN | C | 55 | 64.541 | 3.682 | 64.814 | 1.00 | 62.44 | O |
| ATOM | 12940 | NE2 | GLN | C | 55 | 62.663 | 4.155 | 65.977 | 1.00 | 55.40 | N |
| ATOM | 12943 | C | GLN | C | 55 | 65.197 | 6.491 | 68.835 | 1.00 | 54.46 | C |
| ATOM | 12944 | O | GLN | C | 55 | 66.185 | 6.363 | 69.567 | 1.00 | 57.35 | O |
| ATOM | 12945 | N | LEU | C | 56 | 65.157 | 7.368 | 67.828 | 1.00 | 51.48 | N |
| ATOM | 12947 | CA | LEU | C | 56 | 66.331 | 8.196 | 67.506 | 1.00 | 50.79 | C |
| ATOM | 12949 | CB | LEU | C | 56 | 66.500 | 8.354 | 65.983 | 1.00 | 51.27 | C |
| ATOM | 12952 | CG | LEU | C | 56 | 66.445 | 7.101 | 65.101 | 1.00 | 51.47 | C |
| ATOM | 12954 | CD1 | LEU | C | 56 | 66.780 | 7.437 | 63.654 | 1.00 | 48.56 | C |
| ATOM | 12958 | CD2 | LEU | C | 56 | 67.364 | 6.071 | 65.629 | 1.00 | 47.53 | C |
| ATOM | 12962 | C | LEU | C | 56 | 66.333 | 9.594 | 68.071 | 1.00 | 46.70 | C |
| ATOM | 12963 | O | LEU | C | 56 | 67.129 | 10.385 | 67.639 | 1.00 | 50.22 | O |
| ATOM | 12964 | N | SER | C | 57 | 65.453 | 9.904 | 69.012 | 1.00 | 48.97 | N |
| ATOM | 12966 | CA | SER | C | 57 | 65.298 | 11.260 | 69.565 | 1.00 | 45.66 | C |
| ATOM | 12968 | CB | SER | C | 57 | 63.841 | 11.680 | 69.338 | 1.00 | 44.35 | C |
| ATOM | 12971 | OG | SER | C | 57 | 63.487 | 12.916 | 69.932 | 1.00 | 48.13 | O |
| ATOM | 12973 | C | SER | C | 57 | 65.585 | 11.166 | 71.061 | 1.00 | 45.21 | C |
| ATOM | 12974 | O | SER | C | 57 | 64.960 | 10.361 | 71.721 | 1.00 | 46.15 | O |
| ATOM | 12975 | N | SER | C | 58 | 66.513 | 11.955 | 71.608 | 1.00 | 45.62 | N |
| ATOM | 12977 | CA | SER | C | 58 | 66.828 | 11.885 | 73.068 | 1.00 | 44.99 | C |
| ATOM | 12979 | CB | SER | C | 58 | 68.186 | 12.471 | 73.363 | 1.00 | 39.39 | C |
| ATOM | 12982 | OG | SER | C | 58 | 68.146 | 13.786 | 72.887 | 1.00 | 39.14 | O |
| ATOM | 12984 | C | SER | C | 58 | 65.869 | 12.689 | 73.911 | 1.00 | 44.75 | C |
| ATOM | 12985 | O | SER | C | 58 | 65.676 | 12.433 | 75.101 | 1.00 | 48.81 | O |
| ATOM | 12986 | N | THR | C | 59 | 65.272 | 13.684 | 73.297 | 1.00 | 44.46 | N |
| ATOM | 12988 | CA | THR | C | 59 | 64.320 | 14.493 | 73.998 | 1.00 | 44.45 | C |
| ATOM | 12990 | CB | THR | C | 59 | 64.257 | 15.835 | 73.328 | 1.00 | 39.83 | C |
| ATOM | 12992 | OG1 | THR | C | 59 | 63.953 | 15.610 | 71.960 | 1.00 | 44.67 | O |
| ATOM | 12994 | CG2 | THR | C | 59 | 65.592 | 16.504 | 73.267 | 1.00 | 42.08 | C |
| ATOM | 12998 | C | THR | C | 59 | 62.910 | 13.889 | 73.942 | 1.00 | 49.34 | C |
| ATOM | 12999 | O | THR | C | 59 | 61.953 | 14.574 | 74.329 | 1.00 | 60.35 | O |
| ATOM | 13000 | N | TYR | C | 60 | 62.732 | 12.652 | 73.481 | 1.00 | 45.28 | N |
| ATOM | 13002 | CA | TYR | C | 60 | 61.378 | 12.126 | 73.373 | 1.00 | 42.73 | C |
| ATOM | 13004 | CB | TYR | C | 60 | 61.255 | 11.078 | 72.235 | 1.00 | 44.34 | C |
| ATOM | 13007 | CG | TYR | C | 60 | 60.194 | 10.045 | 72.509 | 1.00 | 39.04 | C |
| ATOM | 13008 | CD1 | TYR | C | 60 | 58.876 | 10.239 | 72.078 | 1.00 | 35.41 | C |
| ATOM | 13010 | CE1 | TYR | C | 60 | 57.873 | 9.308 | 72.344 | 1.00 | 37.20 | C |
| ATOM | 13012 | CZ | TYR | C | 60 | 58.178 | 8.161 | 73.048 | 1.00 | 48.24 | C |
| ATOM | 13013 | OH | TYR | C | 60 | 57.188 | 7.246 | 73.312 | 1.00 | 56.91 | O |
| ATOM | 13015 | CE2 | TYR | C | 60 | 59.496 | 7.934 | 73.495 | 1.00 | 47.13 | C |
| ATOM | 13017 | CD2 | TYR | C | 60 | 60.496 | 8.895 | 73.223 | 1.00 | 35.96 | C |
| ATOM | 13019 | C | TYR | C | 60 | 61.040 | 11.487 | 74.690 | 1.00 | 47.68 | C |
| ATOM | 13020 | O | TYR | C | 60 | 61.858 | 10.721 | 75.189 | 1.00 | 47.26 | O |
| ATOM | 13021 | N | ARG | C | 61 | 59.843 | 11.823 | 75.211 | 1.00 | 48.05 | N |
| ATOM | 13023 | CA | ARG | C | 61 | 59.216 | 11.303 | 76.438 | 1.00 | 46.98 | C |
| ATOM | 13025 | CB | ARG | C | 61 | 58.773 | 12.440 | 77.347 | 1.00 | 49.63 | C |
| ATOM | 13028 | CG | ARG | C | 61 | 59.774 | 12.823 | 78.369 | 1.00 | 60.48 | C |
| ATOM | 13031 | CD | ARG | C | 61 | 60.050 | 11.712 | 79.429 | 1.00 | 66.22 | C |
| ATOM | 13034 | NE | ARG | C | 61 | 60.843 | 10.550 | 78.950 | 1.00 | 69.25 | N |
| ATOM | 13036 | CZ | ARG | C | 61 | 62.129 | 10.585 | 78.575 | 1.00 | 73.53 | C |
| ATOM | 13037 | NH1 | ARG | C | 61 | 62.838 | 11.717 | 78.591 | 1.00 | 76.07 | N |
| ATOM | 13040 | NH2 | ARG | C | 61 | 62.719 | 9.472 | 78.174 | 1.00 | 74.09 | N |
| ATOM | 13043 | C | ARG | C | 61 | 57.932 | 10.601 | 76.126 | 1.00 | 46.33 | C |
| ATOM | 13044 | O | ARG | C | 61 | 57.148 | 11.136 | 75.381 | 1.00 | 41.21 | O |
| ATOM | 13045 | N | ASP | C | 62 | 57.694 | 9.431 | 76.724 | 1.00 | 52.19 | N |
| ATOM | 13047 | CA | ASP | C | 62 | 56.483 | 8.630 | 76.471 | 1.00 | 53.39 | C |
| ATOM | 13049 | CB | ASP | C | 62 | 56.850 | 7.160 | 76.486 | 1.00 | 54.99 | C |
| ATOM | 13052 | CG | ASP | C | 62 | 55.795 | 6.279 | 75.881 | 1.00 | 58.08 | C |
| ATOM | 13053 | OD1 | ASP | C | 62 | 54.599 | 6.664 | 75.822 | 1.00 | 57.84 | O |
| ATOM | 13054 | OD2 | ASP | C | 62 | 56.086 | 5.151 | 75.442 | 1.00 | 62.01 | O |
| ATOM | 13055 | C | ASP | C | 62 | 55.442 | 8.874 | 77.549 | 1.00 | 52.07 | C |
| ATOM | 13056 | O | ASP | C | 62 | 55.779 | 8.845 | 78.699 | 1.00 | 48.18 | O |
| ATOM | 13057 | N | LEU | C | 63 | 54.192 | 9.116 | 77.150 | 1.00 | 57.80 | N |
| ATOM | 13059 | CA | LEU | C | 63 | 53.075 | 9.402 | 78.057 | 1.00 | 56.98 | C |
| ATOM | 13061 | CB | LEU | C | 63 | 52.160 | 10.437 | 77.404 | 1.00 | 57.29 | C |
| ATOM | 13064 | CG | LEU | C | 63 | 52.776 | 11.820 | 77.240 | 1.00 | 58.93 | C |
| ATOM | 13066 | CD1 | LEU | C | 63 | 51.756 | 12.771 | 76.610 | 1.00 | 58.33 | C |
| ATOM | 13070 | CD2 | LEU | C | 63 | 53.270 | 12.356 | 78.568 | 1.00 | 56.05 | C |
| ATOM | 13074 | C | LEU | C | 63 | 52.234 | 8.183 | 78.511 | 1.00 | 57.00 | C |
| ATOM | 13075 | O | LEU | C | 63 | 51.489 | 8.276 | 79.475 | 1.00 | 56.10 | O |
| ATOM | 13076 | N | ARG | C | 64 | 52.323 | 7.053 | 77.828 | 1.00 | 60.31 | N |
| ATOM | 13078 | CA | ARG | C | 64 | 51.702 | 5.821 | 78.337 | 1.00 | 63.72 | C |
| ATOM | 13080 | CB | ARG | C | 64 | 52.093 | 5.634 | 79.801 | 1.00 | 66.02 | C |
| ATOM | 13083 | CG | ARG | C | 64 | 53.077 | 4.540 | 80.025 | 1.00 | 71.85 | C |
| ATOM | 13086 | CD | ARG | C | 64 | 54.241 | 4.949 | 80.865 | 1.00 | 76.21 | C |
| ATOM | 13089 | NE | ARG | C | 64 | 55.478 | 4.473 | 80.255 | 1.00 | 83.39 | N |
| ATOM | 13091 | CZ | ARG | C | 64 | 55.840 | 4.763 | 79.018 | 1.00 | 86.32 | C |
| ATOM | 13092 | NH1 | ARG | C | 64 | 55.045 | 5.527 | 78.271 | 1.00 | 87.82 | N |
| ATOM | 13095 | NH2 | ARG | C | 64 | 56.989 | 4.300 | 78.528 | 1.00 | 87.10 | N |
| ATOM | 13098 | C | ARG | C | 64 | 50.204 | 5.790 | 78.293 | 1.00 | 63.01 | C |
| ATOM | 13099 | O | ARG | C | 64 | 49.556 | 5.513 | 79.292 | 1.00 | 67.08 | O |
| ATOM | 13100 | N | LYS | C | 65 | 49.639 | 6.056 | 77.135 | 1.00 | 62.20 | N |
| ATOM | 13102 | CA | LYS | C | 65 | 48.203 | 6.201 | 77.021 | 1.00 | 56.21 | C |
| ATOM | 13104 | CB | LYS | C | 65 | 47.742 | 7.574 | 77.559 | 1.00 | 53.38 | C |
| ATOM | 13107 | CG | LYS | C | 65 | 46.223 | 7.711 | 77.701 | 1.00 | 61.59 | C |
| ATOM | 13110 | CD | LYS | C | 65 | 45.772 | 8.749 | 78.771 | 1.00 | 65.72 | C |
| ATOM | 13113 | CE | LYS | C | 65 | 46.070 | 10.201 | 78.354 | 1.00 | 68.02 | C |
| ATOM | 13116 | NZ | LYS | C | 65 | 45.991 | 11.186 | 79.492 | 1.00 | 69.89 | N |
| ATOM | 13120 | C | LYS | C | 65 | 48.062 | 6.104 | 75.552 | 1.00 | 50.29 | C |
| ATOM | 13121 | O | LYS | C | 65 | 48.950 | 6.506 | 74.846 | 1.00 | 42.57 | O |
| ATOM | 13122 | N | GLY | C | 66 | 46.965 | 5.552 | 75.088 | 1.00 | 53.19 | N |
| ATOM | 13124 | CA | GLY | C | 66 | 46.748 | 5.388 | 73.668 | 1.00 | 53.88 | C |
| ATOM | 13127 | C | GLY | C | 66 | 45.712 | 6.406 | 73.280 | 1.00 | 55.77 | C |
| ATOM | 13128 | O | GLY | C | 66 | 44.967 | 6.899 | 74.141 | 1.00 | 61.89 | O |
| ATOM | 13129 | N | VAL | C | 67 | 45.654 | 6.713 | 71.993 | 1.00 | 52.57 | N |
| ATOM | 13131 | CA | VAL | C | 67 | 44.753 | 7.726 | 71.494 | 1.00 | 52.31 | C |
| ATOM | 13133 | CB | VAL | C | 67 | 45.485 | 9.074 | 71.457 | 1.00 | 56.46 | C |
| ATOM | 13135 | CG1 | VAL | C | 67 | 46.912 | 8.861 | 71.015 | 1.00 | 61.44 | C |
| ATOM | 13139 | CG2 | VAL | C | 67 | 44.798 | 10.083 | 70.564 | 1.00 | 59.86 | C |
| ATOM | 13143 | C | VAL | C | 67 | 44.323 | 7.234 | 70.146 | 1.00 | 45.56 | C |
| ATOM | 13144 | O | VAL | C | 67 | 45.009 | 6.468 | 69.525 | 1.00 | 50.56 | O |
| ATOM | 13145 | N | TYR | C | 68 | 43.162 | 7.646 | 69.703 | 1.00 | 46.81 | N |
| ATOM | 13147 | CA | TYR | C | 68 | 42.612 | 7.210 | 68.435 | 1.00 | 49.00 | C |
| ATOM | 13149 | CB | TYR | C | 68 | 41.772 | 5.909 | 68.608 | 1.00 | 53.23 | C |
| ATOM | 13152 | CG | TYR | C | 68 | 40.433 | 6.120 | 69.280 | 1.00 | 57.32 | C |
| ATOM | 13153 | CD1 | TYR | C | 68 | 39.298 | 6.444 | 68.537 | 1.00 | 61.05 | C |
| ATOM | 13155 | CE1 | TYR | C | 68 | 38.076 | 6.653 | 69.151 | 1.00 | 62.40 | C |
| ATOM | 13157 | CZ | TYR | C | 68 | 37.980 | 6.524 | 70.533 | 1.00 | 65.60 | C |
| ATOM | 13158 | OH | TYR | C | 68 | 36.814 | 6.708 | 71.234 | 1.00 | 73.33 | O |
| ATOM | 13160 | CE2 | TYR | C | 68 | 39.076 | 6.200 | 71.277 | 1.00 | 68.48 | C |
| ATOM | 13162 | CD2 | TYR | C | 68 | 40.302 | 6.000 | 70.658 | 1.00 | 61.58 | C |
| ATOM | 13164 | C | TYR | C | 68 | 41.731 | 8.358 | 68.011 | 1.00 | 45.30 | C |
| ATOM | 13165 | O | TYR | C | 68 | 41.033 | 8.953 | 68.837 | 1.00 | 41.65 | O |
| ATOM | 13166 | N | VAL | C | 69 | 41.749 | 8.698 | 66.735 | 1.00 | 45.31 | N |
| ATOM | 13168 | CA | VAL | C | 69 | 40.903 | 9.802 | 66.283 | 1.00 | 40.69 | C |
| ATOM | 13170 | CB | VAL | C | 69 | 41.736 | 11.055 | 66.122 | 1.00 | 38.52 | C |
| ATOM | 13172 | CG1 | VAL | C | 69 | 40.861 | 12.229 | 65.749 | 1.00 | 42.88 | C |
| ATOM | 13176 | CG2 | VAL | C | 69 | 42.426 | 11.348 | 67.410 | 1.00 | 38.40 | C |
| ATOM | 13180 | C | VAL | C | 69 | 40.159 | 9.561 | 64.990 | 1.00 | 38.40 | C |
| ATOM | 13181 | O | VAL | C | 69 | 40.759 | 9.309 | 63.978 | 1.00 | 36.05 | O |
| ATOM | 13182 | N | PRO | C | 70 | 38.837 | 9.625 | 65.047 | 1.00 | 41.06 | N |
| ATOM | 13183 | CA | PRO | C | 70 | 37.978 | 9.597 | 63.862 | 1.00 | 41.06 | C |
| ATOM | 13185 | CB | PRO | C | 70 | 36.731 | 8.856 | 64.378 | 1.00 | 39.33 | C |
| ATOM | 13188 | CG | PRO | C | 70 | 36.868 | 8.792 | 65.844 | 1.00 | 34.86 | C |
| ATOM | 13191 | CD | PRO | C | 70 | 38.030 | 9.668 | 66.268 | 1.00 | 41.35 | C |
| ATOM | 13194 | C | PRO | C | 70 | 37.582 | 11.001 | 63.320 | 1.00 | 43.22 | C |
| ATOM | 13195 | O | PRO | C | 70 | 37.305 | 11.927 | 64.102 | 1.00 | 41.52 | O |
| ATOM | 13196 | N | TYR | C | 71 | 37.561 | 11.119 | 61.985 | 1.00 | 48.43 | N |
| ATOM | 13198 | CA | TYR | C | 71 | 37.249 | 12.328 | 61.206 | 1.00 | 45.43 | C |
| ATOM | 13200 | CB | TYR | C | 71 | 38.446 | 12.615 | 60.317 | 1.00 | 47.57 | C |
| ATOM | 13203 | CG | TYR | C | 71 | 39.761 | 12.912 | 61.024 | 1.00 | 45.81 | C |
| ATOM | 13204 | CD1 | TYR | C | 71 | 39.997 | 14.125 | 61.636 | 1.00 | 46.17 | C |
| ATOM | 13206 | CE1 | TYR | C | 71 | 41.203 | 14.358 | 62.268 | 1.00 | 46.12 | C |
| ATOM | 13208 | CZ | TYR | C | 71 | 42.180 | 13.407 | 62.281 | 1.00 | 42.63 | C |
| ATOM | 13209 | OH | TYR | C | 71 | 43.393 | 13.608 | 62.893 | 1.00 | 42.50 | O |
| ATOM | 13211 | CE2 | TYR | C | 71 | 41.963 | 12.246 | 61.683 | 1.00 | 44.84 | C |
| ATOM | 13213 | CD2 | TYR | C | 71 | 40.770 | 11.993 | 61.062 | 1.00 | 47.18 | C |
| ATOM | 13215 | C | TYR | C | 71 | 36.034 | 12.055 | 60.270 | 1.00 | 48.45 | C |
| ATOM | 13216 | O | TYR | C | 71 | 35.606 | 10.915 | 60.053 | 1.00 | 45.82 | O |
| ATOM | 13217 | N | THR | C | 72 | 35.456 | 13.081 | 59.683 | 1.00 | 47.92 | N |
| ATOM | 13219 | CA | THR | C | 72 | 34.356 | 12.805 | 58.760 | 1.00 | 45.15 | C |
| ATOM | 13221 | CB | THR | C | 72 | 34.027 | 14.021 | 57.966 | 1.00 | 40.98 | C |
| ATOM | 13223 | OG1 | THR | C | 72 | 33.809 | 15.111 | 58.867 | 1.00 | 39.12 | O |
| ATOM | 13225 | CG2 | THR | C | 72 | 32.706 | 13.846 | 57.309 | 1.00 | 48.15 | C |
| ATOM | 13229 | C | THR | C | 72 | 34.709 | 11.612 | 57.857 | 1.00 | 45.22 | C |
| ATOM | 13230 | O | THR | C | 72 | 33.884 | 19.719 | 57.657 | 1.00 | 47.99 | O |
| ATOM | 13231 | N | GLN | C | 73 | 35.919 | 11.612 | 57.302 | 1.00 | 47.77 | N |
| ATOM | 13233 | CA | GLN | C | 73 | 36.473 | 10.435 | 56.643 | 1.00 | 48.67 | C |
| ATOM | 13235 | CB | GLN | C | 73 | 36.522 | 10.536 | 55.123 | 1.00 | 48.78 | C |
| ATOM | 13238 | CG | GLN | C | 73 | 35.190 | 10.873 | 54.510 | 1.00 | 58.10 | C |
| ATOM | 13241 | CD | GLN | C | 73 | 34.661 | 9.825 | 53.533 | 1.00 | 65.47 | C |
| ATOM | 13242 | OE1 | GLN | C | 73 | 34.197 | 10.163 | 52.435 | 1.00 | 72.75 | O |
| ATOM | 13243 | NE2 | GLN | C | 73 | 34.701 | 8.555 | 53.939 | 1.00 | 71.25 | N |
| ATOM | 13246 | C | GLN | C | 73 | 37.866 | 10.291 | 57.206 | 1.00 | 53.92 | C |
| ATOM | 13247 | O | GLN | C | 73 | 38.559 | 11.293 | 57.431 | 1.00 | 59.91 | O |
| ATOM | 13248 | N | GLY | C | 74 | 38.274 | 9.049 | 57.462 | 1.00 | 55.15 | N |
| ATOM | 13250 | CA | GLY | C | 74 | 39.616 | 8.758 | 57.943 | 1.00 | 45.87 | C |
| ATOM | 13253 | C | GLY | C | 74 | 39.679 | 8.620 | 59.445 | 1.00 | 44.69 | C |
| ATOM | 13254 | O | GLY | C | 74 | 38.937 | 9.283 | 60.166 | 1.00 | 46.21 | O |
| ATOM | 13255 | N | LYS | C | 75 | 40.548 | 7.729 | 59.907 | 1.00 | 42.25 | N |
| ATOM | 13257 | CA | LYS | C | 75 | 40.894 | 7.639 | 61.306 | 1.00 | 40.58 | C |
| ATOM | 13259 | CB | LYS | C | 75 | 39.809 | 6.919 | 62.124 | 1.00 | 48.07 | C |
| ATOM | 13262 | CG | LYS | C | 75 | 39.614 | 5.420 | 61.883 | 1.00 | 48.51 | C |
| ATOM | 13265 | CD | LYS | C | 75 | 38.232 | 4.997 | 62.417 | 1.00 | 56.60 | C |
| ATOM | 13268 | CE | LYS | C | 75 | 37.077 | 5.271 | 61.414 | 1.00 | 56.69 | C |
| ATOM | 13271 | NZ | LYS | C | 75 | 36.653 | 4.074 | 60.594 | 1.00 | 52.24 | N |
| ATOM | 13275 | C | LYS | C | 75 | 42.270 | 6.970 | 61.410 | 1.00 | 41.75 | C |
| ATOM | 13276 | O | LYS | C | 75 | 42.739 | 6.338 | 60.471 | 1.00 | 36.13 | O |
| ATOM | 13277 | N | TRP | C | 76 | 42.928 | 7.114 | 62.547 | 1.00 | 40.64 | N |
| ATOM | 13279 | CA | TRP | C | 76 | 44.226 | 6.491 | 62.759 | 1.00 | 41.83 | C |
| ATOM | 13281 | CB | TRP | C | 76 | 45.383 | 7.434 | 62.347 | 1.00 | 46.53 | C |
| ATOM | 13284 | CG | TRP | C | 76 | 45.243 | 8.876 | 62.701 | 1.00 | 48.68 | C |
| ATOM | 13285 | CD1 | TRP | C | 76 | 44.953 | 9.877 | 61.845 | 1.00 | 52.07 | C |
| ATOM | 13287 | NE1 | TRP | C | 76 | 44.909 | 11.087 | 62.505 | 1.00 | 48.68 | N |
| ATOM | 13289 | CE2 | TRP | C | 76 | 45.183 | 10.869 | 63.828 | 1.00 | 35.18 | C |
| ATOM | 13290 | CD2 | TRP | C | 76 | 45.401 | 9.492 | 63.992 | 1.00 | 46.84 | C |
| ATOM | 13291 | CE3 | TRP | C | 76 | 45.705 | 9.007 | 65.273 | 1.00 | 46.09 | C |
| ATOM | 13293 | CZ3 | TRP | C | 76 | 45.773 | 9.884 | 66.311 | 1.00 | 41.49 | C |
| ATOM | 13295 | CH2 | TRP | C | 76 | 45.548 | 11.258 | 66.099 | 1.00 | 43.75 | C |
| ATOM | 13297 | CZ2 | TRP | C | 76 | 45.254 | 11.751 | 64.859 | 1.00 | 38.46 | C |
| ATOM | 13299 | C | TRP | C | 76 | 44.298 | 6.196 | 64.227 | 1.00 | 41.30 | C |
| ATOM | 13300 | O | TRP | C | 76 | 43.547 | 6.756 | 65.015 | 1.00 | 44.46 | O |
| ATOM | 13301 | N | GLU | C | 77 | 45.182 | 5.313 | 64.634 | 1.00 | 45.94 | N |
| ATOM | 13303 | CA | GLU | C | 77 | 45.296 | 5.053 | 66.052 | 1.00 | 48.17 | C |
| ATOM | 13305 | CB | GLU | C | 77 | 44.775 | 3.663 | 66.408 | 1.00 | 52.51 | C |
| ATOM | 13308 | CG | GLU | C | 77 | 44.214 | 3.526 | 67.827 | 1.00 | 59.61 | C |
| ATOM | 13311 | CD | GLU | C | 77 | 43.153 | 2.429 | 67.948 | 1.00 | 63.65 | C |
| ATOM | 13312 | OE1 | GLU | C | 77 | 42.966 | 1.729 | 66.889 | 1.00 | 61.90 | O |
| ATOM | 13313 | OE2 | GLU | C | 77 | 42.540 | 2.277 | 69.099 | 1.00 | 61.88 | O |
| ATOM | 13314 | C | GLU | C | 77 | 46.744 | 5.240 | 66.353 | 1.00 | 44.15 | C |
| ATOM | 13315 | O | GLU | C | 77 | 47.580 | 5.038 | 65.481 | 1.00 | 50.71 | O |
| ATOM | 13316 | N | GLY | C | 78 | 47.044 | 5.661 | 67.572 | 1.00 | 45.58 | N |
| ATOM | 13318 | CA | GLY | C | 78 | 48.423 | 5.866 | 67.994 | 1.00 | 45.17 | C |
| ATOM | 13321 | C | GLY | C | 78 | 48.639 | 5.842 | 69.506 | 1.00 | 47.59 | C |
| ATOM | 13322 | O | GLY | C | 78 | 47.757 | 5.539 | 70.298 | 1.00 | 51.18 | O |
| ATOM | 13323 | N | GLU | C | 79 | 49.850 | 6.195 | 69.897 | 1.00 | 49.48 | N |
| ATOM | 13325 | CA | GLU | C | 79 | 50.272 | 6.234 | 71.275 | 1.00 | 48.73 | C |
| ATOM | 13327 | CB | GLU | C | 79 | 51.404 | 5.178 | 71.418 | 1.00 | 56.64 | C |
| ATOM | 13330 | CG | GLU | C | 79 | 51.511 | 4.299 | 70.145 | 1.00 | 57.50 | C |
| ATOM | 13333 | CD | GLU | C | 79 | 52.118 | 2.920 | 70.337 | 1.00 | 61.57 | C |
| ATOM | 13334 | OE1 | GLU | C | 79 | 51.371 | 1.881 | 70.186 | 1.00 | 64.71 | O |
| ATOM | 13335 | OE2 | GLU | C | 79 | 53.349 | 2.881 | 70.607 | 1.00 | 68.25 | O |
| ATOM | 13336 | C | GLU | C | 79 | 50.759 | 7.688 | 71.503 | 1.00 | 47.19 | C |
| ATOM | 13337 | O | GLU | C | 79 | 51.353 | 8.279 | 70.596 | 1.00 | 48.26 | O |
| ATOM | 13338 | N | LEU | C | 80 | 50.507 | 8.280 | 72.669 | 1.00 | 42.10 | N |
| ATOM | 13340 | CA | LEU | C | 80 | 50.942 | 9.647 | 72.904 | 1.00 | 39.01 | C |
| ATOM | 13342 | CB | LEU | C | 80 | 50.042 | 10.306 | 73.887 | 1.00 | 41.35 | C |
| ATOM | 13345 | CG | LEU | C | 80 | 48.580 | 10.390 | 73.740 | 1.00 | 42.44 | C |
| ATOM | 13347 | CD1 | LEU | C | 80 | 48.100 | 10.134 | 75.134 | 1.00 | 42.44 | C |
| ATOM | 13351 | CD2 | LEU | C | 80 | 48.212 | 11.816 | 73.219 | 1.00 | 38.22 | C |
| ATOM | 13355 | C | LEU | C | 80 | 52.283 | 9.789 | 73.570 | 1.00 | 41.97 | C |
| ATOM | 13356 | O | LEU | C | 80 | 52.812 | 8.841 | 74.152 | 1.00 | 41.89 | O |
| ATOM | 13357 | N | GLY | C | 81 | 52.791 | 11.022 | 73.540 | 1.00 | 42.33 | N |
| ATOM | 13359 | CA | GLY | C | 81 | 54.096 | 11.359 | 74.080 | 1.00 | 37.86 | C |
| ATOM | 13362 | C | GLY | C | 81 | 54.408 | 12.832 | 73.856 | 1.00 | 38.28 | C |
| ATOM | 13363 | O | GLY | C | 81 | 53.588 | 13.564 | 73.363 | 1.00 | 41.07 | O |
| ATOM | 13364 | N | THR | C | 82 | 55.593 | 13.298 | 74.206 | 1.00 | 42.53 | N |
| ATOM | 13366 | CA | THR | C | 82 | 55.918 | 14.658 | 73.863 | 1.00 | 45.55 | C |
| ATOM | 13368 | CB | THR | C | 82 | 55.774 | 15.597 | 75.028 | 1.00 | 47.68 | C |
| ATOM | 13370 | OG1 | THR | C | 82 | 56.874 | 15.384 | 75.898 | 1.00 | 52.01 | O |
| ATOM | 13372 | CG2 | THR | C | 82 | 54.529 | 15.315 | 75.871 | 1.00 | 49.71 | C |
| ATOM | 13376 | C | THR | C | 82 | 57.341 | 14.742 | 73.407 | 1.00 | 46.27 | C |
| ATOM | 13377 | O | THR | C | 82 | 58.187 | 13.956 | 73.830 | 1.00 | 44.96 | O |
| ATOM | 13378 | N | ASP | C | 83 | 57.616 | 15.725 | 72.551 | 1.00 | 43.18 | N |
| ATOM | 13380 | CA | ASP | C | 83 | 58.959 | 15.904 | 72.074 | 1.00 | 39.07 | C |
| ATOM | 13382 | CB | ASP | C | 83 | 59.194 | 14.931 | 70.959 | 1.00 | 35.57 | C |
| ATOM | 13385 | CG | ASP | C | 83 | 60.621 | 14.729 | 70.684 | 1.00 | 45.60 | C |
| ATOM | 13386 | OD1 | ASP | C | 83 | 61.426 | 15.599 | 71.102 | 1.00 | 43.07 | O |
| ATOM | 13387 | OD2 | ASP | C | 83 | 61.036 | 13.724 | 70.063 | 1.00 | 50.85 | O |
| ATOM | 13388 | C | ASP | C | 83 | 59.095 | 17.351 | 71.621 | 1.00 | 41.05 | C |
| ATOM | 13389 | O | ASP | C | 83 | 58.131 | 18.100 | 71.679 | 1.00 | 44.03 | O |
| ATOM | 13390 | N | LEU | C | 84 | 60.277 | 17.758 | 71.173 | 1.00 | 41.47 | N |
| ATOM | 13392 | CA | LEU | C | 84 | 60.484 | 19.150 | 70.752 | 1.00 | 40.67 | C |
| ATOM | 13394 | CB | LEU | C | 84 | 61.977 | 19.519 | 70.806 | 1.00 | 39.63 | C |
| ATOM | 13397 | CG | LEU | C | 84 | 62.501 | 19.477 | 72.235 | 1.00 | 38.29 | C |
| ATOM | 13399 | CD1 | LEU | C | 84 | 63.972 | 19.866 | 72.384 | 1.00 | 41.18 | C |
| ATOM | 13403 | CD2 | LEU | C | 84 | 61.664 | 20.398 | 72.966 | 1.00 | 35.07 | C |
| ATOM | 13407 | C | LEU | C | 84 | 59.986 | 19.298 | 69.361 | 1.00 | 38.41 | C |
| ATOM | 13408 | O | LEU | C | 84 | 60.002 | 18.363 | 68.623 | 1.00 | 46.50 | O |
| ATOM | 13409 | N | VAL | C | 85 | 59.563 | 20.484 | 68.990 | 1.00 | 42.93 | N |
| ATOM | 13411 | CA | VAL | C | 85 | 59.011 | 20.699 | 67.676 | 1.00 | 43.40 | C |
| ATOM | 13413 | CB | VAL | C | 85 | 57.500 | 20.553 | 67.711 | 1.00 | 46.20 | C |
| ATOM | 13415 | CG1 | VAL | C | 85 | 56.968 | 20.747 | 66.352 | 1.00 | 46.59 | C |
| ATOM | 13419 | CG2 | VAL | C | 85 | 57.087 | 19.179 | 68.253 | 1.00 | 45.29 | C |
| ATOM | 13423 | C | VAL | C | 85 | 59.260 | 22.100 | 67.248 | 1.00 | 45.84 | C |
| ATOM | 13424 | O | VAL | C | 85 | 58.967 | 23.024 | 68.017 | 1.00 | 51.25 | O |
| ATOM | 13425 | N | SER | C | 86 | 59.787 | 22.268 | 66.035 | 1.00 | 46.65 | N |
| ATOM | 13427 | CA | SER | C | 86 | 60.049 | 23.583 | 65.454 | 1.00 | 43.45 | C |
| ATOM | 13429 | CB | SER | C | 86 | 61.523 | 23.738 | 65.141 | 1.00 | 49.82 | C |
| ATOM | 13432 | OG | SER | C | 86 | 62.315 | 22.686 | 65.709 | 1.00 | 55.85 | O |
| ATOM | 13434 | C | SER | C | 86 | 59.319 | 23.747 | 64.148 | 1.00 | 46.29 | C |
| ATOM | 13435 | O | SER | C | 86 | 58.902 | 22.798 | 63.540 | 1.00 | 51.68 | O |
| ATOM | 13436 | N | ILE | C | 87 | 59.125 | 24.972 | 63.700 | 1.00 | 52.23 | N |
| ATOM | 13438 | CA | ILE | C | 87 | 58.619 | 25.168 | 62.345 | 1.00 | 49.09 | C |
| ATOM | 13440 | CB | ILE | C | 87 | 57.218 | 25.802 | 62.337 | 1.00 | 45.63 | C |
| ATOM | 13442 | CG1 | ILE | C | 87 | 56.382 | 25.334 | 63.533 | 1.00 | 45.33 | C |
| ATOM | 13445 | CD1 | ILE | C | 87 | 54.875 | 25.472 | 63.339 | 1.00 | 45.27 | C |
| ATOM | 13449 | CG2 | ILE | C | 87 | 56.523 | 25.509 | 61.022 | 1.00 | 47.91 | C |
| ATOM | 13453 | C | ILE | C | 87 | 59.645 | 25.999 | 61.570 | 1.00 | 49.56 | C |
| ATOM | 13454 | O | ILE | C | 87 | 59.723 | 27.206 | 61.765 | 1.00 | 58.01 | O |
| ATOM | 13455 | N | PRO | C | 88 | 60.434 | 25.374 | 60.701 | 1.00 | 50.26 | N |
| ATOM | 13456 | CA | PRO | C | 88 | 61.496 | 26.064 | 59.944 | 1.00 | 51.41 | C |
| ATOM | 13458 | CB | PRO | C | 88 | 61.956 | 25.005 | 58.920 | 1.00 | 51.62 | C |
| ATOM | 13461 | CG | PRO | C | 88 | 61.429 | 23.694 | 59.411 | 1.00 | 48.25 | C |
| ATOM | 13464 | CD | PRO | C | 88 | 60.353 | 23.944 | 60.378 | 1.00 | 48.79 | C |
| ATOM | 13467 | C | PRO | C | 88 | 61.054 | 27.350 | 59.206 | 1.00 | 52.93 | C |
| ATOM | 13468 | O | PRO | C | 88 | 61.716 | 28.356 | 59.329 | 1.00 | 52.58 | O |
| ATOM | 13469 | N | HIS | C | 89 | 59.970 | 27.330 | 58.447 | 1.00 | 52.36 | N |
| ATOM | 13471 | CA | HIS | C | 89 | 59.525 | 28.548 | 57.787 | 1.00 | 50.37 | C |
| ATOM | 13473 | CB | HIS | C | 89 | 59.057 | 28.232 | 56.379 | 1.00 | 50.86 | C |
| ATOM | 13476 | CG | HIS | C | 89 | 60.154 | 27.730 | 55.490 | 1.00 | 56.99 | C |
| ATOM | 13477 | ND1 | HIS | C | 89 | 61.204 | 28.524 | 55.084 | 1.00 | 62.43 | N |
| ATOM | 13479 | CE1 | HIS | C | 89 | 62.012 | 27.830 | 54.307 | 1.00 | 57.84 | C |
| ATOM | 13481 | NE2 | HIS | C | 89 | 61.528 | 26.610 | 54.196 | 1.00 | 56.97 | N |
| ATOM | 13483 | CD2 | HIS | C | 89 | 60.368 | 26.521 | 54.928 | 1.00 | 59.06 | C |
| ATOM | 13485 | C | HIS | C | 89 | 58.422 | 29.176 | 58.631 | 1.00 | 50.27 | C |
| ATOM | 13486 | O | HIS | C | 89 | 57.299 | 29.343 | 58.186 | 1.00 | 51.95 | O |
| ATOM | 13487 | N | GLY | C | 90 | 58.780 | 29.525 | 59.865 | 1.00 | 51.98 | N |
| ATOM | 13489 | CA | GLY | C | 90 | 57.866 | 30.031 | 60.882 | 1.00 | 49.95 | C |
| ATOM | 13492 | C | GLY | C | 90 | 58.685 | 30.687 | 61.980 | 1.00 | 47.79 | C |
| ATOM | 13493 | O | GLY | C | 90 | 59.913 | 30.701 | 61.889 | 1.00 | 57.85 | O |
| ATOM | 13494 | N | PRO | C | 91 | 58.029 | 31.167 | 63.034 | 1.00 | 42.33 | N |
| ATOM | 13495 | CA | PRO | C | 91 | 58.688 | 32.056 | 63.990 | 1.00 | 45.30 | C |
| ATOM | 13497 | CB | PRO | C | 91 | 57.586 | 32.447 | 64.952 | 1.00 | 43.17 | C |
| ATOM | 13500 | CG | PRO | C | 91 | 56.415 | 31.674 | 64.623 | 1.00 | 41.44 | C |
| ATOM | 13503 | CD | PRO | C | 91 | 56.658 | 30.820 | 63.433 | 1.00 | 45.00 | C |
| ATOM | 13506 | C | PRO | C | 91 | 59.751 | 31.213 | 64.620 | 1.00 | 48.09 | C |
| ATOM | 13507 | O | PRO | C | 91 | 59.535 | 30.024 | 64.624 | 1.00 | 54.44 | O |
| ATOM | 13508 | N | GLN | C | 92 | 60.843 | 31.737 | 65.131 | 1.00 | 53.59 | N |
| ATOM | 13510 | CA | GLN | C | 92 | 61.921 | 30.814 | 65.443 | 1.00 | 59.67 | C |
| ATOM | 13512 | CB | GLN | C | 92 | 63.309 | 31.354 | 65.139 | 1.00 | 62.99 | C |
| ATOM | 13515 | CG | GLN | C | 92 | 63.355 | 32.565 | 64.259 | 1.00 | 69.98 | C |
| ATOM | 13518 | CD | GLN | C | 92 | 64.686 | 32.618 | 63.546 | 1.00 | 76.93 | C |
| ATOM | 13519 | OE1 | GLN | C | 92 | 65.246 | 31.554 | 63.191 | 1.00 | 79.49 | O |
| ATOM | 13520 | NE2 | GLN | C | 92 | 65.210 | 33.837 | 63.335 | 1.00 | 74.23 | N |
| ATOM | 13523 | C | GLN | C | 92 | 61.893 | 30.449 | 66.851 | 1.00 | 59.04 | C |
| ATOM | 13524 | O | GLN | C | 92 | 62.643 | 30.978 | 67.638 | 1.00 | 67.08 | O |
| ATOM | 13525 | N | VAL | C | 93 | 61.035 | 29.507 | 67.152 | 1.00 | 57.44 | N |
| ATOM | 13527 | CA | VAL | C | 93 | 60.894 | 29.057 | 68.483 | 1.00 | 58.21 | C |
| ATOM | 13529 | CB | VAL | C | 93 | 59.535 | 29.387 | 68.986 | 1.00 | 60.39 | C |
| ATOM | 13531 | CG1 | VAL | C | 93 | 59.427 | 30.878 | 69.168 | 1.00 | 64.36 | C |
| ATOM | 13535 | CG2 | VAL | C | 93 | 58.494 | 28.878 | 68.008 | 1.00 | 60.84 | C |
| ATOM | 13539 | C | VAL | C | 93 | 61.032 | 27.581 | 68.473 | 1.00 | 59.93 | C |
| ATOM | 13540 | O | VAL | C | 93 | 61.183 | 26.945 | 67.436 | 1.00 | 63.78 | O |
| ATOM | 13541 | N | THR | C | 94 | 60.970 | 27.039 | 69.664 | 1.00 | 60.01 | N |
| ATOM | 13543 | CA | THR | C | 94 | 61.045 | 25.637 | 69.862 | 1.00 | 58.54 | C |
| ATOM | 13545 | CB | THR | C | 94 | 62.448 | 25.299 | 70.252 | 1.00 | 59.14 | C |
| ATOM | 13547 | OG1 | THR | C | 94 | 63.337 | 26.190 | 69.577 | 1.00 | 65.27 | O |
| ATOM | 13549 | CG2 | THR | C | 94 | 62.847 | 23.941 | 69.736 | 1.00 | 60.54 | C |
| ATOM | 13553 | C | THR | C | 94 | 60.123 | 25.447 | 71.023 | 1.00 | 59.67 | C |
| ATOM | 13554 | O | THR | C | 94 | 60.260 | 26.139 | 72.029 | 1.00 | 64.36 | O |
| ATOM | 13555 | N | VAL | C | 95 | 59.170 | 24.539 | 70.905 | 1.00 | 55.96 | N |
| ATOM | 13557 | CA | VAL | C | 95 | 58.299 | 24.281 | 72.036 | 1.00 | 52.71 | C |
| ATOM | 13559 | CB | VAL | C | 95 | 56.881 | 24.835 | 71.815 | 1.00 | 49.07 | C |
| ATOM | 13561 | CG1 | VAL | C | 95 | 56.947 | 26.091 | 71.056 | 1..00 | 53.95 | C |
| ATOM | 13565 | CG2 | VAL | C | 95 | 56.065 | 23.897 | 71.054 | 1.00 | 47.27 | C |
| ATOM | 13569 | C | VAL | C | 95 | 58.193 | 22.811 | 72.218 | 1.00 | 48.65 | C |
| ATOM | 13570 | O | VAL | C | 95 | 58.321 | 22.079 | 71.264 | 1.00 | 52.66 | O |
| ATOM | 13571 | N | ARG | C | 96 | 57.955 | 22.378 | 73.446 | 1.00 | 48.25 | N |
| ATOM | 13573 | CA | ARG | C | 96 | 57.728 | 20.980 | 73.704 | 1.00 | 44.23 | C |
| ATOM | 13575 | CB | ARG | C | 96 | 58.145 | 20.582 | 75.105 | 1.00 | 43.93 | C |
| ATOM | 13578 | CG | ARG | C | 96 | 57.739 | 19.139 | 75.427 | 1.00 | 45.83 | C |
| ATOM | 13581 | CD | ARG | C | 96 | 58.485 | 18.481 | 76.591 | 1.00 | 44.68 | C |
| ATOM | 13584 | NE | ARG | C | 96 | 59.906 | 18.368 | 76.346 | 1.00 | 43.10 | N |
| ATOM | 13586 | CZ | ARG | C | 96 | 60.516 | 17.279 | 75.875 | 1.00 | 44.04 | C |
| ATOM | 13587 | NH1 | ARG | C | 96 | 59.848 | 16.181 | 75.591 | 1.00 | 32.28 | N |
| ATOM | 13590 | NH2 | ARG | C | 96 | 61.827 | 17.292 | 75.698 | 1.00 | 50.62 | N |
| ATOM | 13593 | C | ARG | C | 96 | 56.242 | 20.824 | 73.546 | 1.00 | 45.00 | C |
| ATOM | 13594 | O | ARG | C | 96 | 55.491 | 21.695 | 74.008 | 1.00 | 45.85 | O |
| ATOM | 13595 | N | ALA | C | 97 | 55.813 | 19.729 | 72.908 | 1.00 | 43.97 | N |
| ATOM | 13597 | CA | ALA | C | 97 | 54.388 | 19.518 | 72.622 | 1.00 | 42.35 | C |
| ATOM | 13599 | CB | ALA | C | 97 | 54.060 | 20.108 | 71.278 | 1.00 | 42.11 | C |
| ATOM | 13603 | C | ALA | C | 97 | 53.894 | 18.105 | 72.636 | 1.00 | 40.41 | C |
| ATOM | 13604 | O | ALA | C | 97 | 54.575 | 17.166 | 72.319 | 1.00 | 51.69 | O |
| ATOM | 13605 | N | ASN | C | 98 | 52.651 | 17.957 | 72.994 | 1.00 | 46.04 | N |
| ATOM | 13607 | CA | ASN | C | 98 | 52.017 | 16.668 | 72.887 | 1.00 | 44.67 | C |
| ATOM | 13609 | CB | ASN | C | 98 | 50.544 | 16.823 | 73.230 | 1.00 | 44.47 | C |
| ATOM | 13612 | CG | ASN | C | 98 | 50.342 | 17.320 | 74.619 | 1.00 | 44.86 | C |
| ATOM | 13613 | OD1 | ASN | C | 98 | 50.884 | 16.760 | 75.563 | 1.00 | 46.18 | O |
| ATOM | 13614 | ND2 | ASN | C | 98 | 49.564 | 18.378 | 74.762 | 1.00 | 48.48 | N |
| ATOM | 13617 | C | ASN | C | 98 | 52.175 | 16.171 | 71.463 | 1.00 | 41.60 | C |
| ATOM | 13618 | O | ASN | C | 98 | 52.201 | 16.957 | 70.538 | 1.00 | 39.27 | O |
| ATOM | 13619 | N | ILE | C | 99 | 52.269 | 14.866 | 71.286 | 1.00 | 41.90 | N |
| ATOM | 13621 | CA | ILE | C | 99 | 52.403 | 14.307 | 69.954 | 1.00 | 46.94 | C |
| ATOM | 13623 | CB | ILE | C | 99 | 53.861 | 14.330 | 69.470 | 1.00 | 41.20 | C |
| ATOM | 13625 | CG1 | ILE | C | 99 | 54.362 | 15.744 | 69.192 | 1.00 | 46.97 | C |
| ATOM | 13628 | CD1 | ILE | C | 99 | 55.885 | 15.841 | 68.773 | 1.00 | 44.25 | C |
| ATOM | 13632 | CG2 | ILE | C | 99 | 53.939 | 13.652 | 68.168 | 1.00 | 45.91 | C |
| ATOM | 13636 | C | ILE | C | 99 | 51.914 | 12.870 | 69.953 | 1.00 | 47.27 | C |
| ATOM | 13637 | O | ILE | C | 99 | 52.407 | 12.081 | 70.711 | 1.00 | 58.57 | O |
| ATOM | 13638 | N | ALA | C | 100 | 50.944 | 12.554 | 69.116 | 1.00 | 46.89 | N |
| ATOM | 13640 | CA | ALA | C | 100 | 50.418 | 11.219 | 68.962 | 1.00 | 46.93 | C |
| ATOM | 13642 | CB | ALA | C | 100 | 48.949 | 11.274 | 68.540 | 1.00 | 45.92 | C |
| ATOM | 13646 | C | ALA | C | 100 | 51.197 | 10.514 | 67.884 | 1.00 | 48.45 | C |
| ATOM | 13647 | O | ALA | C | 100 | 51.172 | 10.906 | 66.711 | 1.00 | 45.06 | O |
| ATOM | 13648 | N | ALA | C | 101 | 51.872 | 9.436 | 68.250 | 1.00 | 47.39 | N |
| ATOM | 13650 | CA | ALA | C | 101 | 52.639 | 8.732 | 67.260 | 1.00 | 46.43 | C |
| ATOM | 13652 | CB | ALA | C | 101 | 53.820 | 8.041 | 67.940 | 1.00 | 47.14 | C |
| ATOM | 13656 | C | ALA | C | 101 | 51.690 | 7.749 | 66.604 | 1.00 | 41.79 | C |
| ATOM | 13657 | O | ALA | C | 101 | 51.118 | 6.968 | 67.307 | 1.00 | 48.05 | O |
| ATOM | 13658 | N | ILE | C | 102 | 51.500 | 7.781 | 65.285 | 1.00 | 42.66 | N |
| ATOM | 13660 | CA | ILE | C | 102 | 50.507 | 6.878 | 64.635 | 1.00 | 42.52 | C |
| ATOM | 13662 | CB | ILE | C | 102 | 49.916 | 7.478 | 63.366 | 1.00 | 37.68 | C |
| ATOM | 13664 | CG1 | ILE | C | 102 | 49.273 | 8.809 | 63.646 | 1.00 | 42.95 | C |
| ATOM | 13667 | CD1 | ILE | C | 102 | 49.029 | 9.597 | 62.382 | 1.00 | 48.42 | C |
| ATOM | 13671 | CG2 | ILE | C | 102 | 48.845 | 6.590 | 62.789 | 1.00 | 42.11 | C |
| ATOM | 13675 | C | ILE | C | 102 | 51.141 | 5.609 | 64.183 | 1.00 | 42.17 | C |
| ATOM | 13676 | O | ILE | C | 102 | 52.146 | 5.671 | 63.469 | 1.00 | 50.97 | O |
| ATOM | 13677 | N | THR | C | 103 | 50.529 | 4.493 | 64.581 | 1.00 | 39.36 | N |
| ATOM | 13679 | CA | THR | C | 103 | 50.950 | 3.130 | 64.257 | 1.00 | 38.74 | C |
| ATOM | 13681 | CB | THR | C | 103 | 51.024 | 2.324 | 65.542 | 1.00 | 31.31 | C |
| ATOM | 13683 | OG1 | THR | C | 103 | 49.802 | 2.511 | 66.252 | 1.00 | 37.91 | O |
| ATOM | 13685 | CG2 | THR | C | 103 | 51.988 | 2.821 | 66.443 | 1.00 | 25.93 | C |
| ATOM | 13689 | C | THR | C | 103 | 49.936 | 2.306 | 63.434 | 1.00 | 44.91 | C |
| ATOM | 13690 | O | THR | C | 103 | 50.222 | 1.153 | 63.067 | 1.00 | 49.83 | O |
| ATOM | 13691 | N | GLU | C | 104 | 48.764 | 2.866 | 63.161 | 1.00 | 50.27 | N |
| ATOM | 13693 | CA | GLU | C | 104 | 47.674 | 2.128 | 62.535 | 1.00 | 52.57 | C |
| ATOM | 13695 | CB | GLU | C | 104 | 46.967 | 1.315 | 63.591 | 1.00 | 58.34 | C |
| ATOM | 13698 | CG | GLU | C | 104 | 47.546 | −0.048 | 63.935 | 1.00 | 69.41 | C |
| ATOM | 13701 | CD | GLU | C | 104 | 46.428 | −0.983 | 64.324 | 1.00 | 78.52 | C |
| ATOM | 13702 | OE1 | GLU | C | 104 | 45.341 | −0.435 | 64.642 | 1.00 | 80.85 | O |
| ATOM | 13703 | OE2 | GLU | C | 104 | 46.629 | −2.229 | 64.313 | 1.00 | 82.93 | O |
| ATOM | 13704 | C | GLU | C | 104 | 46.607 | 3.068 | 62.052 | 1.00 | 48.07 | C |
| ATOM | 13705 | O | GLU | C | 104 | 46.134 | 3.888 | 62.835 | 1.00 | 35.83 | O |
| ATOM | 13706 | N | SER | C | 105 | 46.185 | 2.970 | 60.800 | 1.00 | 49.78 | N |
| ATOM | 13708 | CA | SER | C | 105 | 45.155 | 3.896 | 60.329 | 1.00 | 51.92 | C |
| ATOM | 13710 | CB | SER | C | 105 | 45.802 | 5.173 | 59.821 | 1.00 | 48.19 | C |
| ATOM | 13713 | OG | SER | C | 105 | 46.446 | 4.896 | 58.587 | 1.00 | 50.41 | O |
| ATOM | 13715 | C | SER | C | 105 | 44.336 | 3.307 | 59.195 | 1.00 | 53.97 | C |
| ATOM | 13716 | O | SER | C | 105 | 44.748 | 2.324 | 58.583 | 1.00 | 54.52 | O |
| ATOM | 13717 | N | ASP | C | 106 | 43.190 | 3.933 | 58.931 | 1.00 | 48.85 | N |
| ATOM | 13719 | CA | ASP | C | 106 | 42.327 | 3.568 | 57.825 | 1.00 | 54.43 | C |
| ATOM | 13721 | CB | ASP | C | 106 | 41.117 | 2.695 | 58.274 | 1.00 | 53.40 | C |
| ATOM | 13724 | CG | ASP | C | 106 | 40.419 | 1.971 | 57.076 | 1.00 | 59.23 | C |
| ATOM | 13725 | OD1 | ASP | C | 106 | 41.164 | 1.374 | 56.243 | 1.00 | 60.96 | O |
| ATOM | 13726 | OD2 | ASP | C | 106 | 39.146 | 1.940 | 56.861 | 1.00 | 64.74 | O |
| ATOM | 13727 | C | ASP | C | 106 | 41.882 | 4.891 | 57.127 | 1.00 | 53.64 | C |
| ATOM | 13728 | O | ASP | C | 106 | 41.304 | 5.783 | 57.734 | 1.00 | 51.47 | O |
| ATOM | 13729 | N | LYS | C | 107 | 42.204 | 4.992 | 55.848 | 1.00 | 54.37 | N |
| ATOM | 13731 | CA | LYS | C | 107 | 41.853 | 6.116 | 54.999 | 1.00 | 51.97 | C |
| ATOM | 13733 | CB | LYS | C | 107 | 40.405 | 5.986 | 54.532 | 1.00 | 53.57 | C |
| ATOM | 13736 | CG | LYS | C | 107 | 39.921 | 4.542 | 54.124 | 1.00 | 55.39 | C |
| ATOM | 13739 | CD | LYS | C | 107 | 38.335 | 4.466 | 54.249 | 1.00 | 59.86 | C |
| ATOM | 13742 | CE | LYS | C | 107 | 37.629 | 3.148 | 53.820 | 1.00 | 61.09 | C |
| ATOM | 13745 | NZ | LYS | C | 107 | 36.060 | 3.234 | 53.879 | 1.00 | 53.27 | N |
| ATOM | 13749 | C | LYS | C | 107 | 42.133 | 7.499 | 55.610 | 1.00 | 50.56 | C |
| ATOM | 13750 | O | LYS | C | 107 | 41.383 | 8.440 | 55.412 | 1.00 | 51.18 | O |
| ATOM | 13751 | N | PHE | C | 108 | 43.223 | 7.613 | 56.350 | 1.00 | 48.34 | N |
| ATOM | 13753 | CA | PHE | C | 108 | 43.712 | 8.905 | 56.787 | 1.00 | 47.27 | C |
| ATOM | 13755 | CB | PHE | C | 108 | 44.540 | 8.748 | 58.031 | 1.00 | 45.62 | C |
| ATOM | 13758 | CG | PHE | C | 108 | 44.938 | 10.029 | 58.643 | 1.00 | 37.04 | C |
| ATOM | 13759 | CD1 | PHE | C | 108 | 43.993 | 10.892 | 59.105 | 1.00 | 38.32 | C |
| ATOM | 13761 | CE1 | PHE | C | 108 | 44.362 | 12.075 | 59.688 | 1.00 | 40.30 | C |
| ATOM | 13763 | CZ | PHE | C | 108 | 45.663 | 12.392 | 59.804 | 1.00 | 33.49 | C |
| ATOM | 13765 | CE2 | PHE | C | 108 | 46.603 | 11.535 | 59.347 | 1.00 | 37.53 | C |
| ATOM | 13767 | CD2 | PHE | C | 108 | 46.237 | 10.357 | 58.770 | 1.00 | 36.71 | C |
| ATOM | 13769 | C | PHE | C | 108 | 44.660 | 9.440 | 55.747 | 1.00 | 50.97 | C |
| ATOM | 13770 | O | PHE | C | 108 | 44.408 | 10.462 | 55.088 | 1.00 | 53.65 | O |
| ATOM | 13771 | N | PHE | C | 109 | 45.768 | 8.718 | 55.612 | 1.00 | 47.95 | N |
| ATOM | 13773 | CA | PHE | C | 109 | 46.805 | 9.096 | 54.701 | 1.00 | 47.94 | C |
| ATOM | 13775 | CB | PHE | C | 109 | 48.006 | 8.198 | 54.915 | 1.00 | 45.29 | C |
| ATOM | 13778 | CG | PHE | C | 109 | 48.678 | 8.429 | 56.255 | 1.00 | 46.27 | C |
| ATOM | 13779 | CD1 | PHE | C | 109 | 49.046 | 7.389 | 57.063 | 1.00 | 48.64 | C |
| ATOM | 13781 | CE1 | PHE | C | 109 | 49.637 | 7.618 | 58.276 | 1.00 | 47.73 | C |
| ATOM | 13783 | CZ | PHE | C | 109 | 49.873 | 8.893 | 58.689 | 1.00 | 48.46 | C |
| ATOM | 13785 | CE2 | PHE | C | 109 | 49.518 | 9.933 | 57.901 | 1.00 | 48.66 | C |
| ATOM | 13787 | CD2 | PHE | C | 109 | 48.922 | 9.707 | 56.698 | 1.00 | 49.42 | C |
| ATOM | 13789 | C | PHE | C | 109 | 46.226 | 9.031 | 53.309 | 1.00 | 53.22 | C |
| ATOM | 13790 | O | PHE | C | 109 | 45.213 | 8.362 | 53.118 | 1.00 | 60.08 | O |
| ATOM | 13791 | N | ILE | C | 110 | 46.836 | 9.748 | 52.359 | 1.00 | 52.83 | N |
| ATOM | 13793 | CA | ILE | C | 110 | 46.374 | 9.790 | 50.976 | 1.00 | 52.12 | C |
| ATOM | 13795 | CB | ILE | C | 110 | 45.836 | 11.149 | 50.688 | 1.00 | 53.27 | C |
| ATOM | 13797 | CG1 | ILE | C | 110 | 44.566 | 11.404 | 51.445 | 1.00 | 52.71 | C |
| ATOM | 13800 | CD1 | ILE | C | 110 | 44.141 | 12.836 | 51.288 | 1.00 | 50.55 | C |
| ATOM | 13804 | CG2 | ILE | C | 110 | 45.504 | 11.291 | 49.236 | 1.00 | 57.99 | C |
| ATOM | 13808 | C | ILE | C | 110 | 47.519 | 9.590 | 50.019 | 1.00 | 54.78 | C |
| ATOM | 13809 | O | ILE | C | 110 | 48.434 | 10.408 | 49.980 | 1.00 | 60.18 | O |
| ATOM | 13810 | N | GLN | C | 111 | 47.485 | 8.529 | 49.227 | 1.00 | 58.31 | N |
| ATOM | 13812 | CA | GLN | C | 111 | 48.617 | 8.239 | 48.348 | 1.00 | 58.73 | C |
| ATOM | 13814 | CB | GLN | C | 111 | 48.255 | 7.125 | 47.389 | 1.00 | 61.62 | C |
| ATOM | 13817 | CG | GLN | C | 111 | 49.245 | 6.964 | 46.243 | 1.00 | 66.55 | C |
| ATOM | 13820 | CD | GLN | C | 111 | 48.738 | 5.997 | 45.183 | 1.00 | 67.87 | C |
| ATOM | 13821 | OE1 | GLN | C | 111 | 49.529 | 5.413 | 44.450 | 1.00 | 66.00 | O |
| ATOM | 13822 | NE2 | GLN | C | 111 | 47.415 | 5.837 | 45.099 | 1.00 | 65.39 | N |
| ATOM | 13825 | C | GLN | C | 111 | 49.047 | 9.471 | 47.560 | 1.00 | 58.27 | C |
| ATOM | 13826 | O | GLN | C | 111 | 48.222 | 10.128 | 46.924 | 1.00 | 57.77 | O |
| ATOM | 13827 | N | GLY | C | 112 | 50.339 | 9.788 | 47.628 | 1.00 | 58.53 | N |
| ATOM | 13829 | CA | GLY | C | 112 | 50.915 | 10.916 | 46.916 | 1.00 | 59.25 | C |
| ATOM | 13832 | C | GLY | C | 112 | 50.726 | 12.291 | 47.540 | 1.00 | 58.52 | C |
| ATOM | 13833 | O | GLY | C | 112 | 51.104 | 13.284 | 46.941 | 1.00 | 54.33 | O |
| ATOM | 13834 | N | SER | C | 113 | 50.158 | 12.356 | 48.739 | 1.00 | 59.61 | N |
| ATOM | 13836 | CA | SER | C | 113 | 49.832 | 13.635 | 49.370 | 1.00 | 59.93 | C |
| ATOM | 13838 | CB | SER | C | 113 | 49.012 | 13.380 | 50.616 | 1.00 | 61.27 | C |
| ATOM | 13841 | OG | SER | C | 113 | 49.791 | 12.666 | 51.583 | 1.00 | 61.67 | O |
| ATOM | 13843 | C | SER | C | 113 | 51.044 | 14.401 | 49.826 | 1.00 | 59.74 | C |
| ATOM | 13844 | O | SER | C | 113 | 50.970 | 15.560 | 50.136 | 1.00 | 64.41 | O |
| ATOM | 13845 | N | ASN | C | 114 | 52.168 | 13.730 | 49.879 | 1.00 | 60.55 | N |
| ATOM | 13847 | CA | ASN | C | 114 | 53.389 | 14.312 | 50.410 | 1.00 | 58.90 | C |
| ATOM | 13849 | CB | ASN | C | 114 | 53.911 | 15.426 | 49.496 | 1.00 | 57.44 | C |
| ATOM | 13852 | CG | ASN | C | 114 | 55.402 | 15.668 | 49.693 | 1.00 | 62.94 | C |
| ATOM | 13853 | OD1 | ASN | C | 114 | 56.179 | 14.725 | 49.898 | 1.00 | 58.01 | O |
| ATOM | 13854 | ND2 | ASN | C | 114 | 55.807 | 16.931 | 49.657 | 1.00 | 67.46 | N |
| ATOM | 13857 | C | ASN | C | 114 | 53.420 | 14.734 | 51.918 | 1.00 | 52.44 | C |
| ATOM | 13858 | O | ASN | C | 114 | 54.474 | 15.175 | 52.388 | 1.00 | 53.11 | O |
| ATOM | 13859 | N | TRP | C | 115 | 52.322 | 14.581 | 52.662 | 1.00 | 48.49 | N |
| ATOM | 13861 | CA | TRP | C | 115 | 52.311 | 14.866 | 54.131 | 1.00 | 47.90 | C |
| ATOM | 13863 | CB | TRP | C | 115 | 51.158 | 15.786 | 54.572 | 1.00 | 44.85 | C |
| ATOM | 13866 | CG | TRP | C | 115 | 49.757 | 15.522 | 54.098 | 1.00 | 35.78 | C |
| ATOM | 13867 | CD1 | TRP | C | 115 | 49.177 | 16.027 | 52.986 | 1.00 | 38.12 | C |
| ATOM | 13869 | NE1 | TRP | C | 115 | 47.879 | 15.592 | 52.868 | 1.00 | 39.72 | N |
| ATOM | 13871 | CE2 | TRP | C | 115 | 47.596 | 14.785 | 53.936 | 1.00 | 41.98 | C |
| ATOM | 13872 | CD2 | TRP | C | 115 | 48.762 | 14.723 | 54.734 | 1.00 | 40.24 | C |
| ATOM | 13873 | CE3 | TRP | C | 115 | 48.733 | 13.947 | 55.898 | 1.00 | 36.07 | C |
| ATOM | 13875 | CZ3 | TRP | C | 115 | 47.597 | 13.286 | 56.223 | 1.00 | 31.67 | C |
| ATOM | 13877 | CH2 | TRP | C | 115 | 46.448 | 13.366 | 55.414 | 1.00 | 41.59 | C |
| ATOM | 13879 | CZ2 | TRP | C | 115 | 46.431 | 14.104 | 54.262 | 1.00 | 37.70 | C |
| ATOM | 13881 | C | TRP | C | 115 | 52.333 | 13.594 | 55.024 | 1.00 | 49.13 | C |
| ATOM | 13882 | O | TRP | C | 115 | 51.893 | 12.529 | 54.610 | 1.00 | 56.05 | O |
| ATOM | 13883 | N | GLU | C | 116 | 52.835 | 13.694 | 56.246 | 1.00 | 42.30 | N |
| ATOM | 13885 | CA | GLU | C | 116 | 53.060 | 12.493 | 57.029 | 1.00 | 36.92 | C |
| ATOM | 13887 | CB | GLU | C | 116 | 54.543 | 12.335 | 57.308 | 1.00 | 35.88 | C |
| ATOM | 13890 | CG | GLU | C | 116 | 55.428 | 11.872 | 56.167 | 1.00 | 40.41 | C |
| ATOM | 13893 | CD | GLU | C | 116 | 56.100 | 12.992 | 55.447 | 1.00 | 43.69 | C |
| ATOM | 13894 | OE1 | GLU | C | 116 | 56.149 | 12.999 | 54.197 | 1.00 | 51.67 | O |
| ATOM | 13895 | OE2 | GLU | C | 116 | 56.596 | 13.871 | 56.142 | 1.00 | 54.68 | O |
| ATOM | 13896 | C | GLU | C | 116 | 52.378 | 12.587 | 58.365 | 1.00 | 42.27 | C |
| ATOM | 13897 | O | GLU | C | 116 | 52.608 | 11.744 | 59.249 | 1.00 | 44.85 | O |
| ATOM | 13898 | N | GLY | C | 117 | 51.547 | 13.609 | 58.518 | 1.00 | 37.85 | N |
| ATOM | 13900 | CA | GLY | C | 117 | 50.997 | 13.960 | 59.807 | 1.00 | 33.97 | C |
| ATOM | 13903 | C | GLY | C | 117 | 50.070 | 15.133 | 59.637 | 1.00 | 37.13 | C |
| ATOM | 13904 | O | GLY | C | 117 | 50.056 | 15.846 | 58.625 | 1.00 | 35.82 | O |
| ATOM | 13905 | N | ILE | C | 118 | 49.256 | 15.338 | 60.636 | 1.00 | 36.24 | N |
| ATOM | 13907 | CA | ILE | C | 118 | 48.332 | 16.425 | 60.572 | 1.00 | 38.55 | C |
| ATOM | 13909 | CB | ILE | C | 118 | 46.910 | 15.921 | 60.803 | 1.00 | 40.07 | C |
| ATOM | 13911 | CG1 | ILE | C | 118 | 45.865 | 16.999 | 60.568 | 1.00 | 42.31 | C |
| ATOM | 13914 | CD1 | ILE | C | 118 | 44.458 | 16.429 | 60.571 | 1.00 | 40.16 | C |
| ATOM | 13918 | CG2 | ILE | C | 118 | 46.748 | 15.448 | 62.229 | 1.00 | 42.62 | C |
| ATOM | 13922 | C | ILE | C | 118 | 48.776 | 17.298 | 61.695 | 1.00 | 41.59 | C |
| ATOM | 13923 | O | ILE | C | 118 | 49.698 | 16.975 | 62.440 | 1.00 | 47.04 | O |
| ATOM | 13924 | N | LEU | C | 119 | 48.111 | 18.419 | 61.807 | 1.00 | 41.76 | N |
| ATOM | 13926 | CA | LEU | C | 119 | 48.341 | 19.372 | 62.878 | 1.00 | 39.99 | C |
| ATOM | 13928 | CB | LEU | C | 119 | 49.449 | 20.360 | 62.540 | 1.00 | 37.22 | C |
| ATOM | 13931 | CG | LEU | C | 119 | 49.390 | 21.634 | 63.399 | 1.00 | 38.87 | C |
| ATOM | 13933 | CD1 | LEU | C | 119 | 49.943 | 21.327 | 64.782 | 1.00 | 34.70 | C |
| ATOM | 13937 | CD2 | LEU | C | 119 | 50.112 | 22.835 | 62.759 | 1.00 | 43.63 | C |
| ATOM | 13941 | C | LEU | C | 119 | 47.033 | 20.104 | 63.031 | 1.00 | 41.75 | C |
| ATOM | 13942 | O | LEU | C | 119 | 46.576 | 20.773 | 62.106 | 1.00 | 46.22 | O |
| ATOM | 13943 | N | GLY | C | 120 | 46.432 | 19.962 | 64.203 | 1.00 | 33.31 | N |
| ATOM | 13945 | CA | GLY | C | 120 | 45.097 | 20.466 | 64.406 | 1.00 | 37.36 | C |
| ATOM | 13948 | C | GLY | C | 120 | 45.145 | 21.751 | 65.149 | 1.00 | 28.62 | C |
| ATOM | 13949 | O | GLY | C | 120 | 45.838 | 21.828 | 66.074 | 1.00 | 30.63 | O |
| ATOM | 13950 | N | LEU | C | 121 | 44.386 | 22.741 | 64.718 | 1.00 | 38.29 | N |
| ATOM | 13952 | CA | LEU | C | 121 | 44.536 | 24.088 | 65.191 | 1.00 | 35.55 | C |
| ATOM | 13954 | CB | LEU | C | 121 | 44.665 | 24.999 | 63.986 | 1.00 | 35.76 | C |
| ATOM | 13957 | CG | LEU | C | 121 | 45.979 | 24.905 | 63.170 | 1.00 | 39.55 | C |
| ATOM | 13959 | CD1 | LEU | C | 121 | 45.900 | 25.748 | 61.887 | 1.00 | 42.96 | C |
| ATOM | 13963 | CD2 | LEU | C | 121 | 47.199 | 25.390 | 63.947 | 1.00 | 36.86 | C |
| ATOM | 13967 | C | LEU | C | 121 | 43.373 | 24.531 | 66.025 | 1.00 | 35.86 | C |
| ATOM | 13968 | O | LEU | C | 121 | 43.358 | 25.666 | 66.534 | 1.00 | 40.24 | O |
| ATOM | 13969 | N | ALA | C | 122 | 42.403 | 23.642 | 66.181 | 1.00 | 33.88 | N |
| ATOM | 13971 | CA | ALA | C | 122 | 41.178 | 23.981 | 66.897 | 1.00 | 33.37 | C |
| ATOM | 13973 | CB | ALA | C | 122 | 40.083 | 23.167 | 66.466 | 1.00 | 40.73 | C |
| ATOM | 13977 | C | ALA | C | 122 | 41.352 | 23.814 | 68.338 | 1.00 | 35.35 | C |
| ATOM | 13978 | O | ALA | C | 122 | 42.347 | 23.300 | 68.812 | 1.00 | 44.15 | O |
| ATOM | 13979 | N | TYR | C | 123 | 40.360 | 24.257 | 69.054 | 1.00 | 49.96 | N |
| ATOM | 13981 | CA | TYR | C | 123 | 40.496 | 24.353 | 70.490 | 1.00 | 46.80 | C |
| ATOM | 13983 | CB | TYR | C | 123 | 39.324 | 25.179 | 71.032 | 1.00 | 45.68 | C |
| ATOM | 13986 | CG | TYR | C | 123 | 39.338 | 26.623 | 70.537 | 1.00 | 44.89 | C |
| ATOM | 13987 | CD1 | TYR | C | 123 | 38.505 | 27.065 | 69.501 | 1.00 | 42.26 | C |
| ATOM | 13989 | CE1 | TYR | C | 123 | 38.537 | 28.386 | 69.072 | 1.00 | 36.64 | C |
| ATOM | 13991 | CZ | TYR | C | 123 | 39.402 | 29.271 | 69.682 | 1.00 | 40.46 | C |
| ATOM | 13992 | OH | TYR | C | 123 | 39.502 | 30.615 | 69.319 | 1.00 | 46.64 | O |
| ATOM | 13994 | CE2 | TYR | C | 123 | 40.211 | 28.849 | 70.689 | 1.00 | 40.22 | C |
| ATOM | 13996 | CD2 | TYR | C | 123 | 40.179 | 27.540 | 71.109 | 1.00 | 43.88 | C |
| ATOM | 13998 | C | TYR | C | 123 | 40.579 | 22.951 | 71.106 | 1.00 | 48.13 | C |
| ATOM | 13999 | O | TYR | C | 123 | 40.430 | 21.933 | 70.415 | 1.00 | 46.62 | O |
| ATOM | 14000 | N | ALA | C | 124 | 40.821 | 22.927 | 72.405 | 1.00 | 48.35 | N |
| ATOM | 14002 | CA | ALA | C | 124 | 40.937 | 21.690 | 73.162 | 1.00 | 51.52 | C |
| ATOM | 14004 | CB | ALA | C | 124 | 41.622 | 21.999 | 74.493 | 1.00 | 51.80 | C |
| ATOM | 14008 | C | ALA | C | 124 | 39.643 | 20.907 | 73.436 | 1.00 | 53.10 | C |
| ATOM | 14009 | O | ALA | C | 124 | 39.713 | 19.728 | 73.722 | 1.00 | 58.47 | O |
| ATOM | 14010 | N | GLU | C | 125 | 38.467 | 21.522 | 73.351 | 1.00 | 52.58 | N |
| ATOM | 14012 | CA | GLU | C | 125 | 37.250 | 20.842 | 73.809 | 1.00 | 48.35 | C |
| ATOM | 14014 | CB | GLU | C | 125 | 36.012 | 21.764 | 73.806 | 1.00 | 45.94 | C |
| ATOM | 14017 | CG | GLU | C | 125 | 34.793 | 21.123 | 74.457 | 1.00 | 51.12 | C |
| ATOM | 14020 | CD | GLU | C | 125 | 33.576 | 22.062 | 74.676 | 1.00 | 65.79 | C |
| ATOM | 14021 | OE1 | GLU | C | 125 | 33.635 | 23.280 | 74.340 | 1.00 | 73.52 | O |
| ATOM | 14022 | OE2 | GLU | C | 125 | 32.521 | 21.580 | 75.191 | 1.00 | 69.28 | O |
| ATOM | 14023 | C | GLU | C | 125 | 36.979 | 19.597 | 72.997 | 1.00 | 49.63 | C |
| ATOM | 14024 | O | GLU | C | 125 | 36.159 | 18.767 | 73.393 | 1.00 | 49.63 | O |
| ATOM | 14025 | N | ILE | C | 126 | 37.653 | 19.474 | 71.855 | 1.00 | 47.96 | N |
| ATOM | 14027 | CA | ILE | C | 126 | 37.477 | 18.324 | 70.969 | 1.00 | 43.11 | C |
| ATOM | 14029 | CB | ILE | C | 126 | 36.849 | 18.722 | 69.649 | 1.00 | 37.68 | C |
| ATOM | 14031 | CG1 | ILE | C | 126 | 37.742 | 19.681 | 68.885 | 1.00 | 42.13 | C |
| ATOM | 14034 | CD1 | ILE | C | 126 | 37.241 | 19.947 | 67.461 | 1.00 | 45.08 | C |
| ATOM | 14038 | CG2 | ILE | C | 126 | 35.632 | 19.390 | 69.898 | 1.00 | 39.52 | C |
| ATOM | 14042 | C | ILE | C | 126 | 38.755 | 17.614 | 70.665 | 1.00 | 41.81 | C |
| ATOM | 14043 | O | ILE | C | 126 | 38.815 | 16.848 | 69.708 | 1.00 | 47.30 | O |
| ATOM | 14044 | N | ALA | C | 127 | 39.789 | 17.856 | 71.447 | 1.00 | 44.88 | N |
| ATOM | 14046 | CA | ALA | C | 127 | 41.005 | 17.060 | 71.286 | 1.00 | 49.29 | C |
| ATOM | 14048 | CB | ALA | C | 127 | 42.164 | 17.690 | 71.997 | 1.00 | 47.42 | C |
| ATOM | 14052 | C | ALA | C | 127 | 40.708 | 15.676 | 71.871 | 1.00 | 53.51 | C |
| ATOM | 14053 | O | ALA | C | 127 | 39.695 | 15.503 | 72.565 | 1.00 | 56.30 | O |
| ATOM | 14054 | N | ARG | C | 128 | 41.570 | 14.700 | 71.591 | 1.00 | 53.29 | N |
| ATOM | 14056 | CA | ARG | C | 128 | 41.396 | 13.345 | 72.102 | 1.00 | 50.59 | C |
| ATOM | 14058 | CB | ARG | C | 128 | 41.159 | 12.348 | 70.949 | 1.00 | 57.97 | C |
| ATOM | 14061 | CG | ARG | C | 128 | 39.759 | 12.300 | 70.183 | 1.00 | 59.94 | C |
| ATOM | 14064 | CD | ARG | C | 128 | 38.475 | 12.155 | 71.017 | 1.00 | 69.66 | C |
| ATOM | 14067 | NE | ARG | C | 128 | 37.271 | 12.355 | 70.198 | 1.00 | 76.55 | N |
| ATOM | 14069 | CZ | ARG | C | 128 | 36.442 | 11.399 | 69.770 | 1.00 | 84.49 | C |
| ATOM | 14070 | NH1 | ARG | C | 128 | 36.641 | 10.106 | 70.062 | 1.00 | 85.61 | N |
| ATOM | 14073 | NH2 | ARG | C | 128 | 35.389 | 11.753 | 69.034 | 1.00 | 86.88 | N |
| ATOM | 14076 | C | ARG | C | 128 | 42.707 | 13.008 | 72.850 | 1.00 | 50.92 | C |
| ATOM | 14077 | O | ARG | C | 128 | 43.799 | 13.530 | 72.544 | 1.00 | 47.14 | O |
| ATOM | 14078 | N | PRO | C | 129 | 42.662 | 12.150 | 73.847 | 1.00 | 45.26 | N |
| ATOM | 14079 | CA | PRO | C | 129 | 41.469 | 11.450 | 74.333 | 1.00 | 42.08 | C |
| ATOM | 14081 | CB | PRO | C | 129 | 42.028 | 10.478 | 75.365 | 1.00 | 43.25 | C |
| ATOM | 14084 | CG | PRO | C | 129 | 43.561 | 10.579 | 75.290 | 1.00 | 38.09 | C |
| ATOM | 14087 | CD | PRO | C | 129 | 43.892 | 11.844 | 74.590 | 1.00 | 35.38 | C |
| ATOM | 14090 | C | PRO | C | 129 | 40.487 | 12.337 | 75.054 | 1.00 | 49.15 | C |
| ATOM | 14091 | O | PRO | C | 129 | 39.322 | 11.949 | 75.129 | 1.00 | 58.20 | O |
| ATOM | 14092 | N | ASP | C | 130 | 40.919 | 13.483 | 75.578 | 1.00 | 52.35 | N |
| ATOM | 14094 | CA | ASP | C | 130 | 40.023 | 14.394 | 76.283 | 1.00 | 55.40 | C |
| ATOM | 14096 | CB | ASP | C | 130 | 39.774 | 13.858 | 77.664 | 1.00 | 61.87 | C |
| ATOM | 14099 | CG | ASP | C | 130 | 41.048 | 13.867 | 78.498 | 1.00 | 70.21 | C |
| ATOM | 14100 | OD1 | ASP | C | 130 | 41.533 | 12.794 | 78.934 | 1.00 | 73.11 | O |
| ATOM | 14101 | OD2 | ASP | C | 130 | 41.650 | 14.930 | 78.752 | 1.00 | 74.28 | O |
| ATOM | 14102 | C | ASP | C | 130 | 40.659 | 15.766 | 76.453 | 1.00 | 55.47 | C |
| ATOM | 14103 | O | ASP | C | 130 | 41.857 | 15.911 | 76.312 | 1.00 | 47.00 | O |
| ATOM | 14104 | N | ASP | C | 131 | 39.835 | 16.755 | 76.804 | 1.00 | 60.76 | N |
| ATOM | 14106 | CA | ASP | C | 131 | 40.235 | 18.173 | 76.853 | 1.00 | 58.88 | C |
| ATOM | 14108 | CB | ASP | C | 131 | 39.051 | 19.107 | 77.223 | 1.00 | 63.07 | C |
| ATOM | 14111 | CG | ASP | C | 131 | 38.290 | 18.660 | 78.474 | 1.00 | 72.14 | C |
| ATOM | 14112 | OD1 | ASP | C | 131 | 38.685 | 17.632 | 79.058 | 1.00 | 82.97 | O |
| ATOM | 14113 | OD2 | ASP | C | 131 | 37.281 | 19.261 | 78.948 | 1.00 | 76.88 | O |
| ATOM | 14114 | C | ASP | C | 131 | 41.415 | 18.554 | 77.696 | 1.00 | 52.31 | C |
| ATOM | 14115 | O | ASP | C | 131 | 41.698 | 19.728 | 77.795 | 1.00 | 60.98 | O |
| ATOM | 14116 | N | SER | C | 132 | 42.116 | 17.620 | 78.304 | 1.00 | 47.46 | N |
| ATOM | 14118 | CA | SER | C | 132 | 43.311 | 17.999 | 79.054 | 1.00 | 45.98 | C |
| ATOM | 14120 | CB | SER | C | 132 | 43.450 | 17.206 | 80.331 | 1.00 | 46.66 | C |
| ATOM | 14123 | OG | SER | C | 132 | 42.859 | 15.924 | 80.195 | 1.00 | 49.20 | O |
| ATOM | 14125 | C | SER | C | 132 | 44.574 | 17.843 | 78.218 | 1.00 | 47.95 | C |
| ATOM | 14126 | O | SER | C | 132 | 45.649 | 18.235 | 78.650 | 1.00 | 52.86 | O |
| ATOM | 14127 | N | LEU | C | 133 | 44.449 | 17.292 | 77.015 | 1.00 | 48.67 | N |
| ATOM | 14129 | CA | LEU | C | 133 | 45.591 | 17.140 | 76.156 | 1.00 | 47.27 | C |
| ATOM | 14131 | CB | LEU | C | 133 | 45.366 | 16.031 | 75.159 | 1.00 | 46.70 | C |
| ATOM | 14134 | CG | LEU | C | 133 | 46.663 | 15.841 | 74.400 | 1.00 | 48.65 | C |
| ATOM | 14136 | CD1 | LEU | C | 133 | 47.713 | 15.384 | 75.385 | 1.00 | 48.76 | C |
| ATOM | 14140 | CD2 | LEU | C | 133 | 46.539 | 14.900 | 73.213 | 1.00 | 50.53 | C |
| ATOM | 14144 | C | LEU | C | 133 | 45.821 | 18.432 | 75.416 | 1.00 | 50.94 | C |
| ATOM | 14145 | O | LEU | C | 133 | 45.405 | 18.612 | 74.271 | 1.00 | 58.31 | O |
| ATOM | 14146 | N | GLU | C | 134 | 46.478 | 19.354 | 76.071 | 1.00 | 51.70 | N |
| ATOM | 14148 | CA | GLU | C | 134 | 46.764 | 20.626 | 75.441 | 1.00 | 56.18 | C |
| ATOM | 14150 | CB | GLU | C | 134 | 47.967 | 21.258 | 76.166 | 1.00 | 56.39 | C |
| ATOM | 14153 | CG | GLU | C | 134 | 48.225 | 22.746 | 75.938 | 1.00 | 59.32 | C |
| ATOM | 14156 | CD | GLU | C | 134 | 49.438 | 23.242 | 76.750 | 1.00 | 65.65 | C |
| ATOM | 14157 | OE1 | GLU | C | 134 | 49.225 | 23.685 | 77.918 | 1.00 | 66.55 | O |
| ATOM | 14158 | OE2 | GLU | C | 134 | 50.605 | 23.183 | 76.240 | 1.00 | 53.92 | O |
| ATOM | 14159 | C | GLU | C | 134 | 47.099 | 20.439 | 73.944 | 1.00 | 55.09 | C |
| ATOM | 14160 | O | GLU | C | 134 | 47.972 | 19.634 | 73.636 | 1.00 | 51.08 | O |
| ATOM | 14161 | N | PRO | C | 135 | 46.399 | 21.161 | 73.042 | 1.00 | 52.45 | N |
| ATOM | 14162 | CA | PRO | C | 135 | 46.789 | 21.308 | 71.624 | 1.00 | 47.35 | C |
| ATOM | 14164 | CB | PRO | C | 135 | 45.590 | 22.018 | 71.001 | 1.00 | 44.20 | C |
| ATOM | 14167 | CG | PRO | C | 135 | 44.524 | 21.918 | 71.952 | 1.00 | 43.96 | C |
| ATOM | 14170 | CD | PRO | C | 135 | 45.135 | 21.861 | 73.310 | 1.00 | 48.87 | C |
| ATOM | 14173 | C | PRO | C | 135 | 48.011 | 22.197 | 71.412 | 1.00 | 45.97 | C |
| ATOM | 14174 | O | PRO | C | 135 | 48.341 | 23.014 | 72.238 | 1.00 | 57.63 | O |
| ATOM | 14175 | N | PHE | C | 136 | 48.665 | 22.030 | 70.271 | 1.00 | 49.91 | N |
| ATOM | 14177 | CA | PHE | C | 136 | 49.918 | 22.693 | 69.912 | 1.00 | 41.68 | C |
| ATOM | 14179 | CB | PHE | C | 136 | 50.230 | 22.292 | 68.497 | 1.00 | 41.45 | C |
| ATOM | 14182 | CG | PHE | C | 136 | 51.358 | 23.034 | 67.882 | 1.00 | 41.29 | C |
| ATOM | 14183 | CD1 | PHE | C | 136 | 52.621 | 22.527 | 67.912 | 1.00 | 37.45 | C |
| ATOM | 14185 | CE1 | PHE | C | 136 | 53.646 | 23.172 | 67.368 | 1.00 | 39.54 | C |
| ATOM | 14187 | CZ | PHE | C | 136 | 53.450 | 24.324 | 66.769 | 1.00 | 46.69 | C |
| ATOM | 14189 | CE2 | PHE | C | 136 | 52.202 | 24.861 | 66.706 | 1.00 | 47.06 | C |
| ATOM | 14191 | CD2 | PHE | C | 136 | 51.152 | 24.216 | 67.266 | 1.00 | 44.15 | C |
| ATOM | 14193 | C | PHE | C | 136 | 49.917 | 24.200 | 69.976 | 1.00 | 48.49 | C |
| ATOM | 14194 | O | PHE | C | 136 | 50.837 | 24.807 | 70.537 | 1.00 | 57.13 | O |
| ATOM | 14195 | N | PHE | C | 137 | 48.902 | 24.838 | 69.403 | 1.00 | 50.04 | N |
| ATOM | 14197 | CA | PHE | C | 137 | 48.879 | 26.300 | 69.407 | 1.00 | 42.38 | C |
| ATOM | 14199 | CB | PHE | C | 137 | 47.751 | 26.868 | 68.559 | 1.00 | 43.63 | C |
| ATOM | 14202 | CG | PHE | C | 137 | 48.105 | 28.174 | 67.943 | 1.00 | 44.76 | C |
| ATOM | 14203 | CD1 | PHE | C | 137 | 48.912 | 28.224 | 66.849 | 1.00 | 48.03 | C |
| ATOM | 14205 | CE1 | PHE | C | 137 | 49.260 | 29.420 | 66.290 | 1.00 | 46.54 | C |
| ATOM | 14207 | CZ | PHE | C | 137 | 48.814 | 30.560 | 66.820 | 1.00 | 44.20 | C |
| ATOM | 14209 | CE2 | PHE | C | 137 | 48.001 | 30.536 | 67.920 | 1.00 | 43.29 | C |
| ATOM | 14211 | CD2 | PHE | C | 137 | 47.648 | 29.358 | 68.474 | 1.00 | 48.04 | C |
| ATOM | 14213 | C | PHE | C | 137 | 48.782 | 26.775 | 70.830 | 1.00 | 45.85 | C |
| ATOM | 14214 | O | PHE | C | 137 | 49.389 | 27.784 | 71.210 | 1.00 | 43.40 | O |
| ATOM | 14215 | N | ASP | C | 138 | 48.034 | 26.044 | 71.651 | 1.00 | 46.65 | N |
| ATOM | 14217 | CA | ASP | C | 138 | 47.918 | 26.481 | 73.024 | 1.00 | 48.49 | C |
| ATOM | 14219 | CB | ASP | C | 138 | 47.010 | 25.594 | 73.849 | 1.00 | 50.42 | C |
| ATOM | 14222 | CG | ASP | C | 138 | 45.550 | 25.771 | 73.523 | 1.00 | 56.72 | C |
| ATOM | 14223 | OD1 | ASP | C | 138 | 44.771 | 26.066 | 74.459 | 1.00 | 57.59 | O |
| ATOM | 14224 | OD2 | ASP | C | 138 | 45.080 | 25.619 | 72.372 | 1.00 | 63.56 | O |
| ATOM | 14225 | C | ASP | C | 138 | 49.321 | 26.472 | 73.611 | 1.00 | 49.79 | C |
| ATOM | 14226 | O | ASP | C | 138 | 49.664 | 27.377 | 74.366 | 1.00 | 50.18 | O |
| ATOM | 14227 | N | SER | C | 139 | 50.117 | 25.459 | 73.256 | 1.00 | 45.66 | N |
| ATOM | 14229 | CA | SER | C | 139 | 51.454 | 25.288 | 73.832 | 1.00 | 45.55 | C |
| ATOM | 14231 | CB | SER | C | 139 | 52.080 | 23.926 | 73.462 | 1.00 | 42.34 | C |
| ATOM | 14234 | OG | SER | C | 139 | 51.244 | 22.831 | 73.802 | 1.00 | 38.35 | O |
| ATOM | 14236 | C | SER | C | 139 | 52.359 | 26.358 | 73.325 | 1.00 | 45.80 | C |
| ATOM | 14237 | O | SER | C | 139 | 53.036 | 27.041 | 74.081 | 1.00 | 47.40 | O |
| ATOM | 14238 | N | LEU | C | 140 | 52.370 | 26.489 | 72.015 | 1.00 | 48.74 | N |
| ATOM | 14240 | CA | LEU | C | 140 | 53.222 | 27.467 | 71.365 | 1.00 | 50.72 | C |
| ATOM | 14242 | CB | LEU | C | 140 | 52.847 | 27.531 | 69.883 | 1.00 | 48.34 | C |
| ATOM | 14245 | CG | LEU | C | 140 | 53.654 | 28.505 | 69.064 | 1.00 | 46.32 | C |
| ATOM | 14247 | CD1 | LEU | C | 140 | 55.129 | 28.109 | 69.092 | 1.00 | 49.60 | C |
| ATOM | 14251 | CD2 | LEU | C | 140 | 53.172 | 28.519 | 67.687 | 1.00 | 46.89 | C |
| ATOM | 14255 | C | LEU | C | 140 | 53.041 | 28.841 | 72.010 | 1.00 | 51.24 | C |
| ATOM | 14256 | O | LEU | C | 140 | 53.989 | 29.558 | 72.257 | 1.00 | 55.86 | O |
| ATOM | 14257 | N | VAL | C | 141 | 51.801 | 29.205 | 72.274 | 1.00 | 53.08 | N |
| ATOM | 14259 | CA | VAL | C | 141 | 51.509 | 30.479 | 72.881 | 1.00 | 50.16 | C |
| ATOM | 14261 | CB | VAL | C | 141 | 50.019 | 30.804 | 72.750 | 1.00 | 51.99 | C |
| ATOM | 14263 | CG1 | VAL | C | 141 | 49.547 | 31.794 | 73.812 | 1.00 | 48.59 | C |
| ATOM | 14267 | CG2 | VAL | C | 141 | 49.751 | 31.368 | 71.362 | 1.00 | 55.31 | C |
| ATOM | 14271 | C | VAL | C | 141 | 51.899 | 30.512 | 74.324 | 1.00 | 52.53 | C |
| ATOM | 14272 | O | VAL | C | 141 | 52.264 | 31.575 | 74.817 | 1.00 | 56.14 | O |
| ATOM | 14273 | N | LYS | C | 142 | 51.828 | 29.375 | 75.019 | 1.00 | 54.25 | N |
| ATOM | 14275 | CA | LYS | C | 142 | 52.179 | 29.360 | 76.439 | 1.00 | 54.82 | C |
| ATOM | 14277 | CB | LYS | C | 142 | 51.575 | 28.156 | 77.211 | 1.00 | 59.67 | C |
| ATOM | 14280 | CG | LYS | C | 142 | 50.151 | 28.411 | 77.893 | 1.00 | 63.35 | C |
| ATOM | 14283 | CD | LYS | C | 142 | 49.565 | 27.179 | 78.677 | 1.00 | 63.97 | C |
| ATOM | 14286 | CE | LYS | C | 142 | 48.278 | 27.516 | 79.548 | 1.00 | 68.51 | C |
| ATOM | 14289 | NZ | LYS | C | 142 | 46.914 | 27.549 | 78.885 | 1.00 | 62.76 | N |
| ATOM | 14293 | C | LYS | C | 142 | 53.692 | 29.426 | 76.618 | 1.00 | 56.79 | C |
| ATOM | 14294 | O | LYS | C | 142 | 54.145 | 30.121 | 77.516 | 1.00 | 63.76 | O |
| ATOM | 14295 | N | GLN | C | 143 | 54.476 | 28.760 | 75.759 | 1.00 | 53.91 | N |
| ATOM | 14297 | CA | GLN | C | 143 | 55.943 | 28.662 | 75.943 | 1.00 | 47.91 | C |
| ATOM | 14299 | CB | GLN | C | 143 | 56.473 | 27.279 | 75.515 | 1.00 | 45.64 | C |
| ATOM | 14302 | CG | GLN | C | 143 | 55.935 | 26.091 | 76.354 | 1.00 | 47.12 | C |
| ATOM | 14305 | CD | GLN | C | 143 | 56.055 | 24.720 | 75.636 | 1.00 | 51.24 | C |
| ATOM | 14306 | OE1 | GLN | C | 143 | 57.100 | 24.396 | 75.047 | 1.00 | 54.46 | O |
| ATOM | 14307 | NE2 | GLN | C | 143 | 54.990 | 23.927 | 75.695 | 1.00 | 34.83 | N |
| ATOM | 14310 | C | GLN | C | 143 | 56.776 | 29.709 | 75.243 | 1.00 | 49.87 | C |
| ATOM | 14311 | O | GLN | C | 143 | 58.000 | 29.605 | 75.260 | 1.00 | 56.15 | O |
| ATOM | 14312 | N | THR | C | 144 | 56.162 | 30.720 | 74.638 | 1.00 | 53.26 | N |
| ATOM | 14314 | CA | THR | C | 144 | 56.945 | 31.754 | 73.943 | 1.00 | 54.99 | C |
| ATOM | 14316 | CB | THR | C | 144 | 57.314 | 31.208 | 72.592 | 1.00 | 55.67 | C |
| ATOM | 14318 | OG1 | THR | C | 144 | 56.128 | 30.775 | 71.926 | 1.00 | 57.59 | O |
| ATOM | 14320 | CG2 | THR | C | 144 | 58.014 | 29.924 | 72.739 | 1.00 | 56.90 | C |
| ATOM | 14324 | C | THR | C | 144 | 56.173 | 33.090 | 73.808 | 1.00 | 57.45 | C |
| ATOM | 14325 | O | THR | C | 144 | 55.095 | 33.229 | 74.379 | 1.00 | 57.38 | O |
| ATOM | 14326 | N | HIS | C | 145 | 56.689 | 34.072 | 73.066 | 1.00 | 57.24 | N |
| ATOM | 14328 | CA | HIS | C | 145 | 55.993 | 35.367 | 72.997 | 1.00 | 59.37 | C |
| ATOM | 14330 | CB | HIS | C | 145 | 56.995 | 36.521 | 73.119 | 1.00 | 62.64 | C |
| ATOM | 14333 | CG | HIS | C | 145 | 57.622 | 36.612 | 74.472 | 1.00 | 63.89 | C |
| ATOM | 14334 | ND1 | HIS | C | 145 | 58.854 | 36.068 | 74.757 | 1.00 | 61.12 | N |
| ATOM | 14336 | CE1 | HIS | C | 145 | 59.142 | 36.279 | 76.028 | 1.00 | 63.96 | C |
| ATOM | 14338 | NE2 | HIS | C | 145 | 58.142 | 36.947 | 76.574 | 1.00 | 67.84 | N |
| ATOM | 14340 | CD2 | HIS | C | 145 | 57.177 | 37.168 | 75.623 | 1.00 | 66.24 | C |
| ATOM | 14342 | C | HIS | C | 145 | 55.106 | 35.584 | 71.768 | 1.00 | 60.03 | C |
| ATOM | 14343 | O | HIS | C | 145 | 54.617 | 36.702 | 71.531 | 1.00 | 61.56 | O |
| ATOM | 14344 | N | VAL | C | 146 | 54.894 | 34.527 | 70.992 | 1.00 | 55.68 | N |
| ATOM | 14346 | CA | VAL | C | 146 | 54.037 | 34.612 | 69.813 | 1.00 | 52.16 | C |
| ATOM | 14348 | CB | VAL | C | 146 | 54.067 | 33.241 | 69.003 | 1.00 | 53.83 | C |
| ATOM | 14350 | CG1 | VAL | C | 146 | 52.899 | 33.085 | 68.012 | 1.00 | 49.94 | C |
| ATOM | 14354 | CG2 | VAL | C | 146 | 55.398 | 33.083 | 68.271 | 1.00 | 49.83 | C |
| ATOM | 14358 | C | VAL | C | 146 | 52.625 | 34.956 | 70.245 | 1.00 | 41.58 | C |
| ATOM | 14359 | O | VAL | C | 146 | 52.071 | 34.277 | 71.047 | 1.00 | 45.36 | O |
| ATOM | 14360 | N | PRO | C | 147 | 52.072 | 36.026 | 69.721 | 1.00 | 38.73 | N |
| ATOM | 14361 | CA | PRO | C | 147 | 50.672 | 36.434 | 69.934 | 1.00 | 44.65 | C |
| ATOM | 14363 | CB | PRO | C | 147 | 50.485 | 37.592 | 68.946 | 1.00 | 47.94 | C |
| ATOM | 14366 | CG | PRO | C | 147 | 51.856 | 38.095 | 68.590 | 1.00 | 46.22 | C |
| ATOM | 14369 | CD | PRO | C | 147 | 52.799 | 36.961 | 68.863 | 1.00 | 48.16 | C |
| ATOM | 14372 | C | PRO | C | 147 | 49.667 | 35.353 | 69.590 | 1.00 | 43.63 | C |
| ATOM | 14373 | O | PRO | C | 147 | 49.987 | 34.428 | 68.854 | 1.00 | 49.34 | O |
| ATOM | 14374 | N | ASN | C | 148 | 48.446 | 35.451 | 70.075 | 1.00 | 44.57 | N |
| ATOM | 14376 | CA | ASN | C | 148 | 47.568 | 34.263 | 69.982 | 1.00 | 43.03 | C |
| ATOM | 14378 | CB | ASN | C | 148 | 46.660 | 34.109 | 71.201 | 1.00 | 38.71 | C |
| ATOM | 14381 | CG | ASN | C | 148 | 46.009 | 32.750 | 71.245 | 1.00 | 40.63 | C |
| ATOM | 14382 | OD1 | ASN | C | 148 | 46.577 | 31.792 | 70.748 | 1.00 | 45.67 | O |
| ATOM | 14383 | ND2 | ASN | C | 148 | 44.812 | 32.656 | 71.820 | 1.00 | 35.19 | N |
| ATOM | 14386 | C | ASN | C | 148 | 46.739 | 34.377 | 68.792 | 1.00 | 44.54 | C |
| ATOM | 14387 | O | ASN | C | 148 | 45.575 | 34.746 | 68.884 | 1.00 | 47.35 | O |
| ATOM | 14388 | N | LEU | C | 149 | 47.324 | 34.076 | 67.649 | 1.00 | 47.80 | N |
| ATOM | 14390 | CA | LEU | C | 149 | 46.625 | 34.355 | 66.423 | 1.00 | 46.06 | C |
| ATOM | 14392 | CB | LEU | C | 149 | 46.483 | 35.861 | 66.342 | 1.00 | 45.20 | C |
| ATOM | 14395 | CG | LEU | C | 149 | 46.167 | 36.389 | 64.967 | 1.00 | 50.04 | C |
| ATOM | 14397 | CD1 | LEU | C | 149 | 45.789 | 37.837 | 65.045 | 1.00 | 50.52 | C |
| ATOM | 14401 | CD2 | LEU | C | 149 | 47.368 | 36.220 | 64.121 | 1.00 | 54.10 | C |
| ATOM | 14405 | C | LEU | C | 149 | 47.370 | 33.856 | 65.209 | 1.00 | 45.78 | C |
| ATOM | 14406 | O | LEU | C | 149 | 48.588 | 34.024 | 65.101 | 1.00 | 48.36 | O |
| ATOM | 14407 | N | PHE | C | 150 | 46.639 | 33.241 | 64.289 | 1.00 | 42.18 | N |
| ATOM | 14409 | CA | PHE | C | 150 | 47.225 | 32.795 | 63.036 | 1.00 | 42.43 | C |
| ATOM | 14411 | CB | PHE | C | 150 | 47.534 | 31.231 | 62.984 | 1.00 | 40.98 | C |
| ATOM | 14414 | CG | PHE | C | 150 | 46.314 | 30.306 | 63.059 | 1.00 | 41.37 | C |
| ATOM | 14415 | CD1 | PHE | C | 150 | 45.783 | 29.924 | 64.283 | 1.00 | 46.98 | C |
| ATOM | 14417 | CE1 | PHE | C | 150 | 44.679 | 29.090 | 64.352 | 1.00 | 40.05 | C |
| ATOM | 14419 | CZ | PHE | C | 150 | 44.101 | 28.628 | 63.198 | 1.00 | 39.23 | C |
| ATOM | 14421 | CE2 | PHE | C | 150 | 44.615 | 28.977 | 61.978 | 1.00 | 32.22 | C |
| ATOM | 14423 | CD2 | PHE | C | 150 | 45.719 | 29.814 | 61.905 | 1.00 | 32.36 | C |
| ATOM | 14425 | C | PHE | C | 150 | 46.286 | 33.330 | 61.950 | 1.00 | 43.38 | C |
| ATOM | 14426 | O | PHE | C | 150 | 45.167 | 33.745 | 62.233 | 1.00 | 48.71 | O |
| ATOM | 14427 | N | SER | C | 151 | 46.759 | 33.329 | 60.720 | 1.00 | 43.07 | N |
| ATOM | 14429 | CA | SER | C | 151 | 45.981 | 33.763 | 59.606 | 1.00 | 37.49 | C |
| ATOM | 14431 | CB | SER | C | 151 | 46.225 | 35.259 | 59.423 | 1.00 | 44.33 | C |
| ATOM | 14434 | OG | SER | C | 151 | 47.595 | 35.588 | 59.289 | 1.00 | 42.06 | O |
| ATOM | 14436 | C | SER | C | 151 | 46.397 | 32.934 | 58.389 | 1.00 | 38.78 | C |
| ATOM | 14437 | O | SER | C | 151 | 47.577 | 32.606 | 58.234 | 1.00 | 36.44 | O |
| ATOM | 14438 | N | LEU | C | 152 | 45.452 | 32.580 | 57.516 | 1.00 | 41.13 | N |
| ATOM | 14440 | CA | LEU | C | 152 | 45.758 | 31.690 | 56.363 | 1.00 | 40.11 | C |
| ATOM | 14442 | CB | LEU | C | 152 | 45.085 | 30.324 | 56.518 | 1.00 | 35.95 | C |
| ATOM | 14445 | CG | LEU | C | 152 | 45.415 | 29.520 | 57.772 | 1.00 | 36.57 | C |
| ATOM | 14447 | CD1 | LEU | C | 152 | 44.398 | 28.489 | 58.070 | 1.00 | 35.32 | C |
| ATOM | 14451 | CD2 | LEU | C | 152 | 46.772 | 28.820 | 57.620 | 1.00 | 44.22 | C |
| ATOM | 14455 | C | LEU | C | 152 | 45.330 | 32.288 | 55.023 | 1.00 | 41.15 | C |
| ATOM | 14456 | O | LEU | C | 152 | 44.319 | 32.983 | 54.936 | 1.00 | 46.18 | O |
| ATOM | 14457 | N | GLN | C | 153 | 46.133 | 32.001 | 54.003 | 1.00 | 40.76 | N |
| ATOM | 14459 | CA | GLN | C | 153 | 45.914 | 32.418 | 52.632 | 1.00 | 36.05 | C |
| ATOM | 14461 | CB | GLN | C | 153 | 46.847 | 33.554 | 52.203 | 1.00 | 39.58 | C |
| ATOM | 14464 | CG | GLN | C | 153 | 46.387 | 34.255 | 50.905 | 1.00 | 46.24 | C |
| ATOM | 14467 | CD | GLN | C | 153 | 47.443 | 35.042 | 50.160 | 1.00 | 49.21 | C |
| ATOM | 14468 | OE1 | GLN | C | 153 | 48.484 | 34.518 | 49.827 | 1.00 | 61.46 | O |
| ATOM | 14469 | NE2 | GLN | C | 153 | 47.155 | 36.304 | 49.870 | 1.00 | 57.83 | N |
| ATOM | 14472 | C | GLN | C | 153 | 46.244 | 31.195 | 51.809 | 1.00 | 34.15 | C |
| ATOM | 14473 | O | GLN | C | 153 | 47.396 | 30.910 | 51.599 | 1.00 | 34.22 | O |
| ATOM | 14474 | N | LEU | C | 154 | 45.219 | 30.487 | 51.348 | 1.00 | 36.48 | N |
| ATOM | 14476 | CA | LEU | C | 154 | 45.351 | 29.289 | 50.558 | 1.00 | 34.16 | C |
| ATOM | 14478 | CB | LEU | C | 154 | 44.251 | 28.339 | 50.981 | 1.00 | 35.94 | C |
| ATOM | 14481 | CG | LEU | C | 154 | 44.128 | 27.928 | 52.434 | 1.00 | 28.19 | C |
| ATOM | 14483 | CD1 | LEU | C | 154 | 43.246 | 26.789 | 52.499 | 1.00 | 34.51 | C |
| ATOM | 14487 | CD2 | LEU | C | 154 | 45.441 | 27.455 | 52.858 | 1.00 | 41.55 | C |
| ATOM | 14491 | C | LEU | C | 154 | 45.144 | 29.591 | 49.075 | 1.00 | 41.40 | C |
| ATOM | 14492 | O | LEU | C | 154 | 44.050 | 29.970 | 48.695 | 1.00 | 47.72 | O |
| ATOM | 14493 | N | CYS | C | 155 | 46.150 | 29.419 | 48.214 | 1.00 | 49.18 | N |
| ATOM | 14495 | CA | CYS | C | 155 | 45.953 | 29.761 | 46.804 | 1.00 | 56.83 | C |
| ATOM | 14497 | CB | CYS | C | 155 | 47.143 | 30.559 | 46.285 | 1.00 | 59.70 | C |
| ATOM | 14500 | SG | CYS | C | 155 | 47.453 | 32.045 | 47.258 | 1.00 | 65.72 | S |
| ATOM | 14501 | C | CYS | C | 155 | 45.685 | 28.565 | 45.895 | 1.00 | 59.52 | C |
| ATOM | 14502 | O | CYS | C | 155 | 46.576 | 27.824 | 45.515 | 1.00 | 59.33 | O |
| ATOM | 14503 | N | GLY | C | 156 | 44.433 | 28.358 | 45.538 | 1.00 | 66.70 | N |
| ATOM | 14505 | CA | GLY | C | 156 | 44.157 | 27.304 | 44.582 | 1.00 | 69.05 | C |
| ATOM | 14508 | C | GLY | C | 156 | 44.971 | 27.693 | 43.359 | 1.00 | 71.96 | C |
| ATOM | 14509 | O | GLY | C | 156 | 45.119 | 28.899 | 43.050 | 1.00 | 71.30 | O |
| ATOM | 14510 | N | ALA | C | 157 | 45.508 | 26.710 | 42.655 | 1.00 | 71.91 | N |
| ATOM | 14512 | CA | ALA | C | 157 | 46.331 | 27.044 | 41.517 | 1.00 | 75.82 | C |
| ATOM | 14514 | CB | ALA | C | 157 | 47.022 | 25.831 | 40.962 | 1.00 | 75.63 | C |
| ATOM | 14518 | C | ALA | C | 157 | 45.458 | 27.645 | 40.467 | 1.00 | 79.16 | C |
| ATOM | 14519 | O | ALA | C | 157 | 45.896 | 28.525 | 39.731 | 1.00 | 85.18 | O |
| ATOM | 14520 | N | GLY | C | 158 | 44.215 | 27.185 | 40.402 | 1.00 | 81.03 | N |
| ATOM | 14522 | CA | GLY | C | 158 | 43.361 | 27.576 | 39.309 | 1.00 | 82.68 | C |
| ATOM | 14525 | C | GLY | C | 158 | 43.851 | 26.802 | 38.095 | 1.00 | 86.22 | C |
| ATOM | 14526 | O | GLY | C | 158 | 43.962 | 27.338 | 36.995 | 1.00 | 88.16 | O |
| ATOM | 14527 | N | PHE | C | 159 | 44.180 | 25.537 | 38.355 | 1.00 | 89.75 | N |
| ATOM | 14529 | CA | PHE | C | 159 | 44.445 | 24.472 | 37.374 | 1.00 | 92.50 | C |
| ATOM | 14531 | CB | PHE | C | 159 | 45.328 | 24.878 | 36.180 | 1.00 | 92.91 | C |
| ATOM | 14534 | CG | PHE | C | 159 | 46.710 | 25.287 | 36.552 | 1.00 | 92.61 | C |
| ATOM | 14535 | CD1 | PHE | C | 159 | 46.993 | 26.621 | 36.810 | 1.00 | 92.40 | C |
| ATOM | 14537 | CE1 | PHE | C | 159 | 48.259 | 27.023 | 37.155 | 1.00 | 93.74 | C |
| ATOM | 14539 | CZ | PHE | C | 159 | 49.284 | 26.084 | 37.244 | 1.00 | 96.27 | C |
| ATOM | 14541 | CE2 | PHE | C | 159 | 49.017 | 24.741 | 36.984 | 1.00 | 95.81 | C |
| ATOM | 14543 | CD2 | PHE | C | 159 | 47.728 | 24.349 | 36.637 | 1.00 | 93.97 | C |
| ATOM | 14545 | C | PHE | C | 159 | 45.035 | 23.300 | 38.180 | 1.00 | 94.38 | C |
| ATOM | 14546 | O | PHE | C | 159 | 45.615 | 23.503 | 39.255 | 1.00 | 93.80 | O |
| ATOM | 14547 | N | PRO | C | 160 | 44.868 | 22.076 | 37.689 | 1.00 | 95.18 | N |
| ATOM | 14548 | CA | PRO | C | 160 | 45.344 | 20.917 | 38.441 | 1.00 | 96.99 | C |
| ATOM | 14550 | CB | PRO | C | 160 | 44.632 | 19.747 | 37.789 | 1.00 | 95.07 | C |
| ATOM | 14553 | CG | PRO | C | 160 | 44.282 | 20.215 | 36.425 | 1.00 | 96.10 | C |
| ATOM | 14556 | CD | PRO | C | 160 | 44.225 | 21.697 | 36.420 | 1.00 | 94.57 | C |
| ATOM | 14559 | C | PRO | C | 160 | 46.848 | 20.733 | 38.357 | 1.00 | 101.28 | C |
| ATOM | 14560 | O | PRO | C | 160 | 47.540 | 21.320 | 37.505 | 1.00 | 104.32 | O |
| ATOM | 14561 | N | LEU | C | 161 | 47.336 | 19.898 | 39.267 | 1.00 | 100.82 | N |
| ATOM | 14563 | CA | LEU | C | 161 | 48.746 | 19.635 | 39.405 | 1.00 | 99.81 | C |
| ATOM | 14565 | CB | LEU | C | 161 | 49.268 | 20.480 | 40.590 | 1.00 | 100.38 | C |
| ATOM | 14568 | CG | LEU | C | 161 | 48.819 | 21.960 | 40.588 | 1.00 | 99.66 | C |
| ATOM | 14570 | CD1 | LEU | C | 161 | 48.883 | 22.650 | 41.957 | 1.00 | 99.34 | C |
| ATOM | 14574 | CD2 | LEU | C | 161 | 49.626 | 22.753 | 39.569 | 1.00 | 100.10 | C |
| ATOM | 14578 | C | LEU | C | 161 | 48.937 | 18.131 | 39.659 | 1.90 | 98.91 | C |
| ATOM | 14579 | O | LEU | C | 161 | 47.989 | 17.432 | 40.017 | 1.00 | 98.25 | O |
| ATOM | 14580 | N | GLN | C | 162 | 50.146 | 17.628 | 39.438 | 1.00 | 97.30 | N |
| ATOM | 14582 | CA | GLN | C | 162 | 50.499 | 16.289 | 39.903 | 1.00 | 98.52 | C |
| ATOM | 14584 | CB | GLN | C | 162 | 51.168 | 15.442 | 38.827 | 1.00 | 97.42 | C |
| ATOM | 14587 | CG | GLN | C | 162 | 50.212 | 14.825 | 37.822 | 1.00 | 98.06 | C |
| ATOM | 14590 | CD | GLN | C | 162 | 50.951 | 14.249 | 36.621 | 1.00 | 96.41 | C |
| ATOM | 14591 | OE1 | GLN | C | 162 | 50.338 | 13.886 | 35.623 | 1.00 | 92.89 | O |
| ATOM | 14592 | NE2 | GLN | C | 162 | 52.277 | 14.173 | 36.721 | 1.00 | 95.15 | N |
| ATOM | 14595 | C | GLN | C | 162 | 51.457 | 16.426 | 41.087 | 1.00 | 98.47 | C |
| ATOM | 14596 | O | GLN | C | 162 | 52.087 | 17.468 | 41.294 | 1.00 | 97.73 | O |
| ATOM | 14597 | N | GLN | C | 163 | 51.559 | 15.352 | 41.853 | 1.00 | 97.05 | N |
| ATOM | 14599 | CA | GLN | C | 163 | 52.407 | 15.302 | 43.033 | 1.00 | 96.01 | C |
| ATOM | 14601 | CB | GLN | C | 163 | 52.669 | 13.818 | 43.343 | 1.00 | 95.60 | C |
| ATOM | 14604 | CG | GLN | C | 163 | 54.092 | 13.337 | 43.578 | 1.00 | 95.45 | C |
| ATOM | 14607 | CD | GLN | C | 163 | 54.107 | 11.824 | 43.872 | 1.00 | 94.18 | C |
| ATOM | 14608 | OE1 | GLN | C | 163 | 54.023 | 11.003 | 42.956 | 1.00 | 91.93 | O |
| ATOM | 14609 | NE2 | GLN | C | 163 | 54.188 | 11.466 | 45.149 | 1.00 | 93.72 | N |
| ATOM | 14612 | C | GLN | C | 163 | 53.681 | 16.191 | 42.973 | 1.00 | 95.28 | C |
| ATOM | 14613 | O | GLN | C | 163 | 53.880 | 17.037 | 43.854 | 1.00 | 95.34 | O |
| ATOM | 14614 | N | SER | C | 164 | 54.528 | 16.041 | 41.956 | 1.00 | 93.34 | N |
| ATOM | 14616 | CA | SER | C | 164 | 55.762 | 16.838 | 41.910 | 1.00 | 93.19 | C |
| ATOM | 14618 | CB | SER | C | 164 | 56.730 | 16.336 | 40.832 | 1.00 | 92.70 | C |
| ATOM | 14621 | OG | SER | C | 164 | 57.775 | 15.565 | 41.411 | 1.00 | 87.37 | O |
| ATOM | 14623 | C | SER | C | 164 | 55.478 | 18.324 | 41.720 | 1.00 | 94.82 | C |
| ATOM | 14624 | O | SER | C | 164 | 56.259 | 19.174 | 42.150 | 1.00 | 95.13 | O |
| ATOM | 14625 | N | GLU | C | 165 | 54.359 | 18.628 | 41.072 | 1.00 | 98.53 | N |
| ATOM | 14627 | CA | GLU | C | 165 | 53.948 | 20.013 | 40.853 | 1.00 | 99.60 | C |
| ATOM | 14629 | CB | GLU | C | 165 | 52.680 | 20.077 | 39.996 | 1.00 | 98.79 | C |
| ATOM | 14632 | CG | GLU | C | 165 | 52.859 | 19.545 | 38.582 | 1.00 | 98.44 | C |
| ATOM | 14635 | CD | GLU | C | 165 | 51.602 | 19.706 | 37.754 | 1.00 | 99.73 | C |
| ATOM | 14636 | OE1 | GLU | C | 165 | 51.122 | 20.853 | 37.657 | 1.00 | 100.81 | O |
| ATOM | 14637 | OE2 | GLU | C | 165 | 51.086 | 18.693 | 37.213 | 1.00 | 99.63 | O |
| ATOM | 14638 | C | GLU | C | 165 | 53.728 | 20.700 | 42.197 | 1.00 | 100.08 | C |
| ATOM | 14639 | O | GLU | C | 165 | 54.080 | 21.873 | 42.375 | 1.00 | 100.95 | O |
| ATOM | 14640 | N | VAL | C | 166 | 53.143 | 19.958 | 43.136 | 1.00 | 100.04 | N |
| ATOM | 14642 | CA | VAL | C | 166 | 52.950 | 20.446 | 44.504 | 1.00 | 99.34 | C |
| ATOM | 14644 | CB | VAL | C | 166 | 52.142 | 19.435 | 45.372 | 1.00 | 98.17 | C |
| ATOM | 14646 | CG1 | VAL | C | 166 | 52.900 | 19.049 | 46.665 | 1.00 | 95.99 | C |
| ATOM | 14650 | CG2 | VAL | C | 166 | 50.743 | 19.987 | 45.665 | 1.00 | 93.72 | C |
| ATOM | 14654 | C | VAL | C | 166 | 54.307 | 20.701 | 45.142 | 1.00 | 100.09 | C |
| ATOM | 14655 | O | VAL | C | 166 | 54.503 | 21.667 | 45.882 | 1.00 | 100.64 | O |
| ATOM | 14656 | N | LEU | C | 167 | 55.258 | 19.829 | 44.855 | 1.00 | 99.30 | N |
| ATOM | 14658 | CA | LEU | C | 167 | 56.547 | 20.023 | 45.434 | 1.00 | 97.76 | C |
| ATOM | 14660 | CB | LEU | C | 167 | 57.558 | 18.968 | 44.958 | 1.00 | 98.11 | C |
| ATOM | 14663 | CG | LEU | C | 167 | 57.227 | 17.539 | 45.438 | 1.00 | 99.43 | C |
| ATOM | 14665 | CD1 | LEU | C | 167 | 58.481 | 16.676 | 45.639 | 1.00 | 97.68 | C |
| ATOM | 14669 | CD2 | LEU | C | 167 | 56.393 | 17.554 | 46.730 | 1.00 | 97.95 | C |
| ATOM | 14673 | C | LEU | C | 167 | 56.977 | 21.424 | 45.079 | 1.00 | 96.62 | C |
| ATOM | 14674 | O | LEU | C | 167 | 57.027 | 22.301 | 45.938 | 1.00 | 97.39 | O |
| ATOM | 14675 | N | ALA | C | 168 | 57.253 | 21.650 | 43.805 | 1.00 | 94.46 | N |
| ATOM | 14677 | CA | ALA | C | 168 | 57.889 | 22.898 | 43.403 | 1.00 | 92.65 | C |
| ATOM | 14679 | CB | ALA | C | 168 | 58.323 | 22.800 | 41.943 | 1.00 | 92.92 | C |
| ATOM | 14683 | C | ALA | C | 168 | 57.082 | 24.191 | 43.654 | 1.00 | 91.29 | C |
| ATOM | 14684 | O | ALA | C | 168 | 57.666 | 25.209 | 44.025 | 1.00 | 89.06 | O |
| ATOM | 14685 | N | SER | C | 169 | 55.761 | 24.159 | 43.471 | 1.00 | 89.36 | N |
| ATOM | 14687 | CA | SER | C | 169 | 54.960 | 25.382 | 43.593 | 1.00 | 89.32 | C |
| ATOM | 14689 | CB | SER | C | 169 | 53.669 | 25.298 | 42.757 | 1.00 | 89.95 | C |
| ATOM | 14692 | OG | SER | C | 169 | 53.966 | 25.200 | 41.370 | 1.00 | 87.54 | O |
| ATOM | 14694 | C | SER | C | 169 | 54.652 | 25.754 | 45.046 | 1.00 | 87.23 | C |
| ATOM | 14695 | O | SER | C | 169 | 54.933 | 24.993 | 45.958 | 1.00 | 85.58 | O |
| ATOM | 14696 | N | VAL | C | 170 | 54.078 | 26.946 | 45.217 | 1.00 | 86.76 | N |
| ATOM | 14698 | CA | VAL | C | 170 | 53.776 | 27.562 | 46.512 | 1.00 | 83.54 | C |
| ATOM | 14700 | CB | VAL | C | 170 | 54.404 | 28.964 | 46.601 | 1.00 | 84.64 | C |
| ATOM | 14702 | CG1 | VAL | C | 170 | 53.931 | 29.684 | 47.870 | 1.00 | 84.38 | C |
| ATOM | 14706 | CG2 | VAL | C | 170 | 55.930 | 28.892 | 46.502 | 1.00 | 85.18 | C |
| ATOM | 14710 | C | VAL | C | 170 | 52.291 | 27.812 | 46.572 | 1.00 | 79.04 | C |
| ATOM | 14711 | O | VAL | C | 170 | 51.788 | 28.676 | 45.840 | 1.00 | 81.45 | O |
| ATOM | 14712 | N | GLY | C | 171 | 51.583 | 27.102 | 47.443 | 1.00 | 71.56 | N |
| ATOM | 14714 | CA | GLY | C | 171 | 50.128 | 27.173 | 47.448 | 1.00 | 64.22 | C |
| ATOM | 14717 | C | GLY | C | 171 | 49.495 | 27.867 | 48.630 | 1.00 | 54.08 | C |
| ATOM | 14718 | O | GLY | C | 171 | 48.314 | 27.778 | 48.813 | 1.00 | 54.31 | O |
| ATOM | 14719 | N | GLY | C | 172 | 50.254 | 28.553 | 49.450 | 1.00 | 45.40 | N |
| ATOM | 14721 | CA | GLY | C | 172 | 49.596 | 29.320 | 50.492 | 1.00 | 44.40 | C |
| ATOM | 14724 | C | GLY | C | 172 | 50.491 | 29.789 | 51.617 | 1.00 | 39.50 | C |
| ATOM | 14725 | O | GLY | C | 172 | 51.682 | 29.496 | 51.654 | 1.00 | 42.06 | O |
| ATOM | 14726 | N | SER | C | 173 | 49.917 | 30.513 | 52.555 | 1.00 | 33.35 | N |
| ATOM | 14728 | CA | SER | C | 173 | 50.723 | 30.976 | 53.651 | 1.00 | 39.63 | C |
| ATOM | 14730 | CB | SER | C | 173 | 51.106 | 32.475 | 53.530 | 1.00 | 36.22 | C |
| ATOM | 14733 | OG | SER | C | 173 | 51.885 | 32.763 | 52.392 | 1.00 | 39.70 | O |
| ATOM | 14735 | C | SER | C | 173 | 50.007 | 30.807 | 54.936 | 1.00 | 39.12 | C |
| ATOM | 14736 | O | SER | C | 173 | 48.860 | 31.214 | 55.043 | 1.00 | 45.16 | O |
| ATOM | 14737 | N | MET | C | 174 | 50.713 | 30.220 | 55.900 | 1.00 | 41.12 | N |
| ATOM | 14739 | CA | MET | C | 174 | 50.303 | 30.226 | 57.291 | 1.00 | 44.55 | C |
| ATOM | 14741 | CB | MET | C | 174 | 50.469 | 28.856 | 57.929 | 1.00 | 45.29 | C |
| ATOM | 14744 | CG | MET | C | 174 | 49.569 | 28.713 | 59.111 | 1.00 | 49.87 | C |
| ATOM | 14747 | SD | MET | C | 174 | 50.002 | 27.521 | 60.286 | 1.00 | 54.93 | S |
| ATOM | 14748 | CE | MET | C | 174 | 51.580 | 27.087 | 59.828 | 1.00 | 51.63 | C |
| ATOM | 14752 | C | MET | C | 174 | 51.198 | 31.220 | 58.045 | 1.00 | 43.36 | C |
| ATOM | 14753 | O | MET | C | 174 | 52.395 | 30.963 | 58.243 | 1.00 | 46.43 | O |
| ATOM | 14754 | N | ILE | C | 175 | 50.621 | 32.339 | 58.460 | 1.00 | 41.87 | N |
| ATOM | 14756 | CA | ILE | C | 175 | 51.342 | 33.356 | 59.222 | 1.00 | 41.16 | C |
| ATOM | 14758 | CB | ILE | C | 175 | 50.853 | 34.763 | 58.898 | 1.00 | 33.28 | C |
| ATOM | 14760 | CG1 | ILE | C | 175 | 50.662 | 34.909 | 57.409 | 1.00 | 34.50 | C |
| ATOM | 14763 | CD1 | ILE | C | 175 | 51.930 | 35.029 | 56.688 | 1.00 | 31.57 | C |
| ATOM | 14767 | CG2 | ILE | C | 175 | 51.848 | 35.783 | 59.266 | 1.00 | 26.95 | C |
| ATOM | 14771 | C | ILE | C | 175 | 51.052 | 33.050 | 60.683 | 1.00 | 47.92 | C |
| ATOM | 14772 | O | ILE | C | 175 | 49.912 | 33.241 | 61.145 | 1.00 | 45.68 | O |
| ATOM | 14773 | N | ILE | C | 176 | 52.066 | 32.530 | 61.386 | 1.00 | 46.95 | N |
| ATOM | 14775 | CA | ILE | C | 176 | 51.953 | 32.289 | 62.810 | 1.00 | 46.06 | C |
| ATOM | 14777 | CB | ILE | C | 176 | 52.958 | 31.333 | 63.304 | 1.00 | 49.65 | C |
| ATOM | 14779 | CG1 | ILE | C | 176 | 52.844 | 30.056 | 62.508 | 1.00 | 49.91 | C |
| ATOM | 14782 | CD1 | ILE | C | 176 | 51.673 | 29.189 | 62.918 | 1.00 | 54.20 | C |
| ATOM | 14786 | CG2 | ILE | C | 176 | 52.730 | 31.133 | 64.803 | 1.00 | 50.08 | C |
| ATOM | 14790 | C | ILE | C | 176 | 52.294 | 33.603 | 63.475 | 1.00 | 48.42 | C |
| ATOM | 14791 | O | ILE | C | 176 | 53.364 | 34.165 | 63.256 | 1.00 | 47.56 | O |
| ATOM | 14792 | N | GLY | C | 177 | 51.388 | 34.085 | 64.296 | 1.00 | 51.03 | N |
| ATOM | 14794 | CA | GLY | C | 177 | 51.639 | 35.267 | 65.073 | 1.00 | 49.29 | C |
| ATOM | 14797 | C | GLY | C | 177 | 51.067 | 36.536 | 64.513 | 1.00 | 49.22 | C |
| ATOM | 14798 | O | GLY | C | 177 | 51.328 | 37.590 | 65.060 | 1.00 | 50.25 | O |
| ATOM | 14799 | N | GLY | C | 178 | 50.297 | 36.517 | 63.436 | 1.00 | 52.12 | N |
| ATOM | 14801 | CA | GLY | C | 178 | 49.818 | 37.836 | 63.033 | 1.00 | 50.89 | C |
| ATOM | 14804 | C | GLY | C | 178 | 48.891 | 38.108 | 61.873 | 1.00 | 48.10 | C |
| ATOM | 14805 | O | GLY | C | 178 | 47.903 | 37.436 | 61.665 | 1.00 | 59.40 | O |
| ATOM | 14806 | N | ILE | C | 179 | 49.206 | 39.172 | 61.138 | 1.00 | 44.57 | N |
| ATOM | 14808 | CA | ILE | C | 179 | 48.455 | 39.544 | 59.927 | 1.00 | 44.27 | C |
| ATOM | 14810 | CB | ILE | C | 179 | 47.304 | 40.507 | 60.254 | 1.00 | 39.02 | C |
| ATOM | 14812 | CG1 | ILE | C | 179 | 46.428 | 39.947 | 61.351 | 1.00 | 36.52 | C |
| ATOM | 14815 | CD1 | ILE | C | 179 | 45.118 | 40.632 | 61.461 | 1.00 | 43.02 | C |
| ATOM | 14819 | CG2 | ILE | C | 179 | 46.476 | 40.788 | 59.032 | 1.00 | 32.12 | C |
| ATOM | 14823 | C | ILE | C | 179 | 49.370 | 40.148 | 58.825 | 1.00 | 43.98 | C |
| ATOM | 14824 | O | ILE | C | 179 | 49.928 | 41.199 | 59.007 | 1.00 | 48.65 | O |
| ATOM | 14825 | N | ASP | C | 180 | 49.510 | 39.482 | 57.681 | 1.00 | 42.18 | N |
| ATOM | 14827 | CA | ASP | C | 180 | 50.325 | 40.046 | 56.639 | 1.00 | 39.96 | C |
| ATOM | 14829 | CB | ASP | C | 180 | 51.142 | 38.993 | 55.913 | 1.00 | 35.38 | C |
| ATOM | 14832 | CG | ASP | C | 180 | 52.216 | 39.643 | 55.073 | 1.00 | 43.62 | C |
| ATOM | 14833 | OD1 | ASP | C | 180 | 53.415 | 39.527 | 55.436 | 1.00 | 41.41 | O |
| ATOM | 14834 | OD2 | ASP | C | 180 | 51.932 | 40.337 | 54.049 | 1.00 | 50.13 | O |
| ATOM | 14835 | C | ASP | C | 180 | 49.596 | 40.945 | 55.639 | 1.00 | 37.85 | C |
| ATOM | 14836 | O | ASP | C | 180 | 48.805 | 40.506 | 54.799 | 1.00 | 48.70 | O |
| ATOM | 14837 | N | HIS | C | 181 | 49.887 | 42.219 | 55.721 | 1.00 | 40.62 | N |
| ATOM | 14839 | CA | HIS | C | 181 | 49.259 | 43.224 | 54.859 | 1.00 | 39.93 | C |
| ATOM | 14841 | CB | HIS | C | 181 | 49.838 | 44.602 | 55.142 | 1.00 | 42.24 | C |
| ATOM | 14844 | CG | HIS | C | 181 | 48.853 | 45.720 | 55.038 | 1.00 | 52.30 | C |
| ATOM | 14845 | ND1 | HIS | C | 181 | 47.791 | 45.856 | 55.906 | 1.00 | 62.73 | N |
| ATOM | 14847 | CE1 | HIS | C | 181 | 47.100 | 46.939 | 55.586 | 1.00 | 64.35 | C |
| ATOM | 14849 | NE2 | HIS | C | 181 | 47.681 | 47.515 | 54.550 | 1.00 | 57.25 | N |
| ATOM | 14851 | CD2 | HIS | C | 181 | 48.779 | 46.772 | 54.188 | 1.00 | 53.26 | C |
| ATOM | 14853 | C | HIS | C | 181 | 49.505 | 42.975 | 53.416 | 1.00 | 36.05 | C |
| ATOM | 14854 | O | HIS | C | 181 | 48.850 | 43.585 | 52.631 | 1.00 | 39.99 | O |
| ATOM | 14855 | N | SER | C | 182 | 50.433 | 42.117 | 53.008 | 1.00 | 32.07 | N |
| ATOM | 14857 | CA | SER | C | 182 | 50.575 | 41.978 | 51.568 | 1.00 | 31.16 | C |
| ATOM | 14859 | CB | SER | C | 182 | 51.990 | 41.690 | 51.146 | 1.00 | 28.15 | C |
| ATOM | 14862 | OG | SER | C | 182 | 52.281 | 40.358 | 51.488 | 1.00 | 36.39 | O |
| ATOM | 14864 | C | SER | C | 182 | 49.735 | 40.850 | 51.087 | 1.00 | 36.53 | C |
| ATOM | 14865 | O | SER | C | 182 | 49.785 | 40.488 | 49.903 | 1.00 | 33.95 | O |
| ATOM | 14866 | N | LEU | C | 183 | 48.945 | 40.264 | 51.970 | 1.00 | 36.48 | N |
| ATOM | 14868 | CA | LEU | C | 183 | 48.242 | 39.094 | 51.515 | 1.00 | 36.94 | C |
| ATOM | 14870 | CB | LEU | C | 183 | 48.284 | 38.048 | 52.585 | 1.00 | 35.90 | C |
| ATOM | 14873 | CG | LEU | C | 183 | 49.701 | 37.713 | 52.907 | 1.00 | 36.59 | C |
| ATOM | 14875 | CD1 | LEU | C | 183 | 49.617 | 36.793 | 54.059 | 1.00 | 44.02 | C |
| ATOM | 14879 | CD2 | LEU | C | 183 | 50.418 | 37.076 | 51.761 | 1.00 | 37.13 | C |
| ATOM | 14883 | C | LEU | C | 183 | 46.834 | 39.379 | 51.089 | 1.00 | 36.63 | C |
| ATOM | 14884 | O | LEU | C | 183 | 46.152 | 38.513 | 50.512 | 1.00 | 37.72 | O |
| ATOM | 14885 | N | TYR | C | 184 | 46.383 | 40.589 | 51.347 | 1.00 | 39.63 | N |
| ATOM | 14887 | CA | TYR | C | 184 | 45.003 | 40.926 | 50.997 | 1.00 | 41.01 | C |
| ATOM | 14889 | CB | TYR | C | 184 | 44.136 | 40.845 | 52.263 | 1.00 | 34.47 | C |
| ATOM | 14892 | CG | TYR | C | 184 | 44.423 | 41.814 | 53.399 | 1.00 | 30.49 | C |
| ATOM | 14893 | CD1 | TYR | C | 184 | 45.456 | 41.627 | 54.266 | 1.00 | 32.99 | C |
| ATOM | 14895 | CE1 | TYR | C | 184 | 45.680 | 42.482 | 55.285 | 1.00 | 35.85 | C |
| ATOM | 14897 | CZ | TYR | C | 184 | 44.853 | 43.570 | 55.459 | 1.00 | 39.95 | C |
| ATOM | 14898 | OH | TYR | C | 184 | 45.049 | 44.511 | 56.484 | 1.00 | 46.78 | O |
| ATOM | 14900 | CE2 | TYR | C | 184 | 43.838 | 43.748 | 54.621 | 1.00 | 32.10 | C |
| ATOM | 14902 | CD2 | TYR | C | 184 | 43.623 | 42.887 | 53.612 | 1.00 | 33.46 | C |
| ATOM | 14904 | C | TYR | C | 184 | 44.845 | 42.263 | 50.216 | 1.00 | 43.54 | C |
| ATOM | 14905 | O | TYR | C | 184 | 45.836 | 42.918 | 49.891 | 1.00 | 47.52 | O |
| ATOM | 14906 | N | THR | C | 185 | 43.609 | 42.626 | 49.881 | 1.00 | 45.07 | N |
| ATOM | 14908 | CA | THR | C | 185 | 43.280 | 43.968 | 49.381 | 1.00 | 47.09 | C |
| ATOM | 14910 | CB | THR | C | 185 | 43.121 | 44.018 | 47.889 | 1.00 | 48.22 | C |
| ATOM | 14912 | OG1 | THR | C | 185 | 42.287 | 42.924 | 47.447 | 1.00 | 52.14 | O |
| ATOM | 14914 | CG2 | THR | C | 185 | 44.474 | 43.823 | 47.207 | 1.00 | 48.12 | C |
| ATOM | 14918 | C | THR | C | 185 | 41.961 | 44.331 | 50.010 | 1.00 | 45.23 | C |
| ATOM | 14919 | O | THR | C | 185 | 41.187 | 43.469 | 50.313 | 1.00 | 51.13 | O |
| ATOM | 14920 | N | GLY | C | 186 | 41.706 | 45.605 | 50.205 | 1.00 | 49.66 | N |
| ATOM | 14922 | CA | GLY | C | 186 | 40.476 | 46.065 | 50.831 | 1.00 | 48.30 | C |
| ATOM | 14925 | C | GLY | C | 186 | 40.560 | 45.905 | 52.325 | 1.00 | 48.55 | C |
| ATOM | 14926 | O | GLY | C | 186 | 41.655 | 45.707 | 52.842 | 1.00 | 47.91 | O |
| ATOM | 14927 | N | SER | C | 187 | 39.396 | 45.958 | 52.981 | 1.00 | 50.74 | N |
| ATOM | 14929 | CA | SER | C | 187 | 39.240 | 45.868 | 54.440 | 1.00 | 52.59 | C |
| ATOM | 14931 | CB | SER | C | 187 | 38.165 | 46.874 | 54.835 | 1.00 | 52.84 | C |
| ATOM | 14934 | OG | SER | C | 187 | 38.765 | 48.164 | 54.996 | 1.00 | 64.09 | O |
| ATOM | 14936 | C | SER | C | 187 | 38.900 | 44.491 | 55.071 | 1.00 | 51.13 | C |
| ATOM | 14937 | O | SER | C | 187 | 38.078 | 43.735 | 54.553 | 1.00 | 58.59 | O |
| ATOM | 14938 | N | LEU | C | 188 | 39.517 | 44.167 | 56.197 | 1.00 | 44.76 | N |
| ATOM | 14940 | CA | LEU | C | 188 | 39.192 | 42.925 | 56.896 | 1.00 | 44.33 | C |
| ATOM | 14942 | CB | LEU | C | 188 | 40.202 | 42.617 | 57.992 | 1.00 | 39.50 | C |
| ATOM | 14945 | CG | LEU | C | 188 | 41.507 | 41.917 | 57.677 | 1.00 | 40.78 | C |
| ATOM | 14947 | CD1 | LEU | C | 188 | 42.459 | 42.055 | 58.880 | 1.00 | 40.19 | C |
| ATOM | 14951 | CD2 | LEU | C | 188 | 41.258 | 40.443 | 57.322 | 1.00 | 36.35 | C |
| ATOM | 14955 | C | LEU | C | 188 | 37.879 | 43.128 | 57.610 | 1.00 | 48.87 | C |
| ATOM | 14956 | O | LEU | C | 188 | 37.694 | 44.169 | 58.250 | 1.00 | 48.46 | O |
| ATOM | 14957 | N | TRP | C | 189 | 36.971 | 42.152 | 57.523 | 1.00 | 50.27 | N |
| ATOM | 14959 | CA | TRP | C | 189 | 35.720 | 42.206 | 58.282 | 1.00 | 46.66 | C |
| ATOM | 14961 | CB | TRP | C | 189 | 34.526 | 42.094 | 57.365 | 1.00 | 49.51 | C |
| ATOM | 14964 | CG | TRP | C | 189 | 34.218 | 43.331 | 56.642 | 1.00 | 46.43 | C |
| ATOM | 14965 | CD1 | TRP | C | 189 | 34.767 | 43.747 | 55.472 | 1.00 | 50.14 | C |
| ATOM | 14967 | NE1 | TRP | C | 189 | 34.226 | 44.953 | 55.103 | 1.00 | 45.63 | N |
| ATOM | 14969 | CE2 | TRP | C | 189 | 33.316 | 45.334 | 56.048 | 1.00 | 42.21 | C |
| ATOM | 14970 | CD2 | TRP | C | 189 | 33.282 | 44.328 | 57.024 | 1.00 | 46.72 | C |
| ATOM | 14971 | CE3 | TRP | C | 189 | 32.415 | 44.487 | 58.110 | 1.00 | 51.29 | C |
| ATOM | 14973 | CZ3 | TRP | C | 189 | 31.629 | 45.612 | 58.171 | 1.00 | 45.32 | C |
| ATOM | 14975 | CH2 | TRP | C | 189 | 31.691 | 46.580 | 57.183 | 1.00 | 43.31 | C |
| ATOM | 14977 | CZ2 | TRP | C | 189 | 32.530 | 46.459 | 56.118 | 1.00 | 40.90 | C |
| ATOM | 14979 | C | TRP | C | 189 | 35.739 | 41.024 | 59.234 | 1.00 | 47.48 | C |
| ATOM | 14980 | O | TRP | C | 189 | 36.164 | 39.955 | 58.850 | 1.00 | 52.47 | O |
| ATOM | 14981 | N | TYR | C | 190 | 35.285 | 41.212 | 60.466 | 1.00 | 47.80 | N |
| ATOM | 14983 | CA | TYR | C | 190 | 35.345 | 40.164 | 61.477 | 1.00 | 49.62 | C |
| ATOM | 14985 | CB | TYR | C | 190 | 36.104 | 40.623 | 62.725 | 1.00 | 51.24 | C |
| ATOM | 14988 | CG | TYR | C | 190 | 37.569 | 40.940 | 62.541 | 1.00 | 54.91 | C |
| ATOM | 14989 | CD1 | TYR | C | 190 | 37.958 | 42.213 | 62.201 | 1.00 | 54.91 | C |
| ATOM | 14991 | CE1 | TYR | C | 190 | 39.281 | 42.539 | 62.037 | 1.00 | 55.50 | C |
| ATOM | 14993 | CZ | TYR | C | 190 | 40.257 | 41.593 | 62.212 | 1.00 | 60.48 | C |
| ATOM | 14994 | OH | TYR | C | 190 | 41.560 | 42.025 | 62.025 | 1.00 | 63.65 | O |
| ATOM | 14996 | CE2 | TYR | C | 190 | 39.909 | 40.309 | 62.557 | 1.00 | 54.45 | C |
| ATOM | 14998 | CD2 | TYR | C | 190 | 38.560 | 39.991 | 62.724 | 1.00 | 51.81 | C |
| ATOM | 15000 | C | TYR | C | 190 | 33.974 | 39.656 | 61.943 | 1.00 | 50.65 | C |
| ATOM | 15001 | O | TYR | C | 190 | 33.088 | 40.459 | 62.254 | 1.00 | 48.81 | O |
| ATOM | 15002 | N | THR | C | 191 | 33.820 | 38.322 | 61.995 | 1.00 | 48.48 | N |
| ATOM | 15004 | CA | THR | C | 191 | 32.600 | 37.689 | 62.484 | 1.00 | 45.23 | C |
| ATOM | 15006 | CB | THR | C | 191 | 32.090 | 36.647 | 61.492 | 1.00 | 43.53 | C |
| ATOM | 15008 | OG1 | THR | C | 191 | 30.809 | 36.160 | 61.917 | 1.00 | 47.87 | O |
| ATOM | 15010 | CG2 | THR | C | 191 | 32.973 | 35.419 | 61.505 | 1.00 | 47.43 | C |
| ATOM | 15014 | C | THR | C | 191 | 33.009 | 37.017 | 63.775 | 1.00 | 43.55 | C |
| ATOM | 15015 | O | THR | C | 191 | 34.143 | 36.540 | 63.858 | 1.00 | 46.67 | O |
| ATOM | 15016 | N | PRO | C | 192 | 32.125 | 36.948 | 64.774 | 1.00 | 43.58 | N |
| ATOM | 15017 | CA | PRO | C | 192 | 32.486 | 36.382 | 66.084 | 1.00 | 40.68 | C |
| ATOM | 15019 | CB | PRO | C | 192 | 31.334 | 36.786 | 66.980 | 1.00 | 35.99 | C |
| ATOM | 15022 | CG | PRO | C | 192 | 30.531 | 37.704 | 66.212 | 1.00 | 39.36 | C |
| ATOM | 15025 | CD | PRO | C | 192 | 30.718 | 37.376 | 64.766 | 1.00 | 43.30 | C |
| ATOM | 15028 | C | PRO | C | 192 | 32.515 | 34.907 | 66.109 | 1.00 | 42.65 | C |
| ATOM | 15029 | O | PRO | C | 192 | 31.684 | 34.307 | 65.446 | 1.00 | 46.37 | O |
| ATOM | 15030 | N | ILE | C | 193 | 33.444 | 34.335 | 66.868 | 1.00 | 49.91 | N |
| ATOM | 15032 | CA | ILE | C | 193 | 33.504 | 32.886 | 67.059 | 1.00 | 49.57 | C |
| ATOM | 15034 | CB | ILE | C | 193 | 34.897 | 32.436 | 67.468 | 1.00 | 51.07 | C |
| ATOM | 15036 | CG1 | ILE | C | 193 | 35.908 | 32.688 | 66.353 | 1.00 | 48.43 | C |
| ATOM | 15039 | CD1 | ILE | C | 193 | 37.370 | 32.489 | 66.806 | 1.00 | 45.97 | C |
| ATOM | 15043 | CG2 | ILE | C | 193 | 34.900 | 30.990 | 67.792 | 1.00 | 52.95 | C |
| ATOM | 15047 | C | ILE | C | 193 | 32.481 | 32.601 | 68.140 | 1.00 | 49.00 | C |
| ATOM | 15048 | O | ILE | C | 193 | 32.546 | 33.112 | 69.244 | 1.00 | 53.60 | O |
| ATOM | 15049 | N | ARG | C | 194 | 31.515 | 31.789 | 67.788 | 1.00 | 51.30 | N |
| ATOM | 15051 | CA | ARG | C | 194 | 30.380 | 31.515 | 68.636 | 1.00 | 52.47 | C |
| ATOM | 15053 | CB | ARG | C | 194 | 29.397 | 30.664 | 67.867 | 1.00 | 50.52 | C |
| ATOM | 15056 | CG | ARG | C | 194 | 28.155 | 30.427 | 68.634 | 1.00 | 54.20 | C |
| ATOM | 15059 | CD | ARG | C | 194 | 27.064 | 29.819 | 67.810 | 1.00 | 52.92 | C |
| ATOM | 15062 | NE | ARG | C | 194 | 25.825 | 29.795 | 68.558 | 1.00 | 52.77 | N |
| ATOM | 15064 | CZ | ARG | C | 194 | 25.026 | 28.743 | 68.642 | 1.00 | 56.49 | C |
| ATOM | 15065 | NH1 | ARG | C | 194 | 25.330 | 27.617 | 68.016 | 1.00 | 54.87 | N |
| ATOM | 15068 | NH2 | ARG | C | 194 | 23.906 | 28.824 | 69.354 | 1.00 | 58.48 | N |
| ATOM | 15071 | C | ARG | C | 194 | 30.745 | 30.746 | 69.870 | 1.00 | 55.75 | C |
| ATOM | 15072 | O | ARG | C | 194 | 30.253 | 31.017 | 70.956 | 1.00 | 57.01 | O |
| ATOM | 15073 | N | ARG | C | 195 | 31.607 | 29.759 | 69.692 | 1.00 | 59.11 | N |
| ATOM | 15075 | CA | ARG | C | 195 | 31.973 | 28.902 | 70.790 | 1.00 | 59.31 | C |
| ATOM | 15077 | CB | ARG | C | 195 | 30.995 | 27.764 | 70.945 | 1.00 | 61.07 | C |
| ATOM | 15080 | CG | ARG | C | 195 | 30.821 | 27.418 | 72.392 | 1.00 | 64.46 | C |
| ATOM | 15083 | CD | ARG | C | 195 | 31.137 | 25.977 | 72.690 | 1.00 | 67.27 | C |
| ATOM | 15086 | NE | ARG | C | 195 | 30.142 | 25.083 | 72.128 | 1.00 | 66.30 | N |
| ATOM | 15088 | CZ | ARG | C | 195 | 30.096 | 23.795 | 72.352 | 1.00 | 63.54 | C |
| ATOM | 15089 | NH1 | ARG | C | 195 | 31.012 | 23.226 | 73.136 | 1.00 | 67.84 | N |
| ATOM | 15092 | NH2 | ARG | C | 195 | 29.136 | 23.080 | 71.786 | 1.00 | 58.10 | N |
| ATOM | 15095 | C | ARG | C | 195 | 33.311 | 28.328 | 70.540 | 1.00 | 57.13 | C |
| ATOM | 15096 | O | ARG | C | 195 | 33.657 | 28.010 | 69.409 | 1.00 | 57.63 | O |
| ATOM | 15097 | N | GLU | C | 196 | 34.064 | 28.179 | 71.614 | 1.00 | 55.49 | N |
| ATOM | 15099 | CA | GLU | C | 196 | 35.439 | 27.772 | 71.492 | 1.00 | 53.91 | C |
| ATOM | 15101 | CB | GLU | C | 196 | 36.266 | 28.390 | 72.627 | 1.00 | 54.10 | C |
| ATOM | 15104 | CG | GLU | C | 196 | 36.813 | 29.768 | 72.252 | 1.00 | 56.99 | C |
| ATOM | 15107 | CD | GLU | C | 196 | 37.389 | 30.542 | 73.417 | 1.00 | 54.97 | C |
| ATOM | 15108 | OE1 | GLU | C | 196 | 37.947 | 29.924 | 74.327 | 1.00 | 59.03 | O |
| ATOM | 15109 | OE2 | GLU | C | 196 | 37.278 | 31.777 | 73.422 | 1.00 | 62.18 | O |
| ATOM | 15110 | C | GLU | C | 196 | 35.569 | 26.287 | 71.468 | 1.00 | 49.41 | C |
| ATOM | 15111 | O | GLU | C | 196 | 35.832 | 25.689 | 72.484 | 1.00 | 58.38 | O |
| ATOM | 15112 | N | TRP | C | 197 | 35.365 | 25.689 | 70.307 | 1.00 | 44.54 | N |
| ATOM | 15114 | CA | TRP | C | 197 | 35.667 | 24.254 | 70.129 | 1.00 | 43.73 | C |
| ATOM | 15116 | CB | TRP | C | 197 | 34.518 | 23.348 | 70.471 | 1.00 | 44.23 | C |
| ATOM | 15119 | CG | TRP | C | 197 | 33.243 | 23.641 | 69.824 | 1.00 | 46.97 | C |
| ATOM | 15120 | CD1 | TRP | C | 197 | 32.774 | 24.851 | 69.402 | 1.00 | 46.93 | C |
| ATOM | 15122 | NE1 | TRP | C | 197 | 31.530 | 24.704 | 68.849 | 1.00 | 39.59 | N |
| ATOM | 15124 | CE2 | TRP | C | 197 | 31.176 | 23.389 | 68.908 | 1.00 | 45.07 | C |
| ATOM | 15125 | CD2 | TRP | C | 197 | 32.237 | 22.696 | 69.520 | 1.00 | 47.29 | C |
| ATOM | 15126 | CE3 | TRP | C | 197 | 32.118 | 21.326 | 69.695 | 1.00 | 44.90 | C |
| ATOM | 15128 | CZ3 | TRP | C | 197 | 30.950 | 20.707 | 69.265 | 1.00 | 46.40 | C |
| ATOM | 15130 | CH2 | TRP | C | 197 | 29.924 | 21.419 | 68.674 | 1.00 | 43.73 | C |
| ATOM | 15132 | CZ2 | TRP | C | 197 | 30.012 | 22.757 | 68.484 | 1.00 | 49.22 | C |
| ATOM | 15134 | C | TRP | C | 197 | 36.159 | 24.039 | 68.713 | 1.00 | 41.47 | C |
| ATOM | 15135 | O | TRP | C | 197 | 37.354 | 24.026 | 68.484 | 1.00 | 40.26 | O |
| ATOM | 15136 | N | TYR | C | 198 | 35.265 | 23.864 | 67.758 | 1.00 | 43.56 | N |
| ATOM | 15138 | CA | TYR | C | 198 | 35.681 | 24.018 | 66.375 | 1.00 | 45.02 | C |
| ATOM | 15140 | CB | TYR | C | 198 | 34.617 | 23.530 | 65.443 | 1.00 | 47.08 | C |
| ATOM | 15143 | CG | TYR | C | 198 | 34.364 | 22.060 | 65.493 | 1.00 | 46.30 | C |
| ATOM | 15144 | CD1 | TYR | C | 198 | 35.062 | 21.207 | 64.639 | 1.00 | 44.86 | C |
| ATOM | 15146 | CE1 | TYR | C | 198 | 34.843 | 19.877 | 64.651 | 1.00 | 43.00 | C |
| ATOM | 15148 | CZ | TYR | C | 198 | 33.911 | 19.347 | 65.520 | 1.00 | 41.88 | C |
| ATOM | 15149 | OH | TYR | C | 198 | 33.750 | 18.004 | 65.494 | 1.00 | 29.11 | O |
| ATOM | 15151 | CE2 | TYR | C | 198 | 33.188 | 20.156 | 66.383 | 1.00 | 44.38 | C |
| ATOM | 15153 | CD2 | TYR | C | 198 | 33.418 | 21.520 | 66.363 | 1.00 | 43.48 | C |
| ATOM | 15155 | C | TYR | C | 198 | 35.802 | 25.526 | 66.232 | 1.00 | 41.33 | C |
| ATOM | 15156 | O | TYR | C | 198 | 35.698 | 26.191 | 67.216 | 1.00 | 46.95 | O |
| ATOM | 15157 | N | TYR | C | 199 | 36.058 | 26.049 | 65.035 | 1.00 | 44.07 | N |
| ATOM | 15159 | CA | TYR | C | 199 | 36.047 | 27.504 | 64.759 | 1.00 | 42.83 | C |
| ATOM | 15161 | CB | TYR | C | 199 | 37.161 | 27.933 | 63.758 | 1.00 | 39.32 | C |
| ATOM | 15164 | CG | TYR | C | 199 | 38.494 | 28.044 | 64.448 | 1.00 | 37.70 | C |
| ATOM | 15165 | CD1 | TYR | C | 199 | 39.421 | 27.041 | 64.346 | 1.00 | 40.50 | C |
| ATOM | 15167 | CE1 | TYR | C | 199 | 40.637 | 27.114 | 65.007 | 1.00 | 42.66 | C |
| ATOM | 15169 | CZ | TYR | C | 199 | 40.932 | 28.211 | 65.793 | 1.00 | 44.12 | C |
| ATOM | 15170 | OH | TYR | C | 199 | 42.147 | 28.239 | 66.426 | 1.00 | 35.21 | O |
| ATOM | 15172 | CE2 | TYR | C | 199 | 40.008 | 29.247 | 65.931 | 1.00 | 37.43 | C |
| ATOM | 15174 | CD2 | TYR | C | 199 | 38.800 | 29.152 | 65.263 | 1.00 | 40.32 | C |
| ATOM | 15176 | C | TYR | C | 199 | 34.647 | 27.733 | 64.200 | 1.00 | 46.28 | C |
| ATOM | 15177 | O | TYR | C | 199 | 34.443 | 27.731 | 62.985 | 1.00 | 45.68 | O |
| ATOM | 15178 | N | GLU | C | 200 | 33.685 | 27.902 | 65.104 | 1.00 | 48.83 | N |
| ATOM | 15180 | CA | GLU | C | 200 | 32.273 | 27.958 | 64.746 | 1.00 | 49.81 | C |
| ATOM | 15182 | CB | GLU | C | 200 | 31.387 | 27.454 | 65.917 | 1.00 | 51.32 | C |
| ATOM | 15185 | CG | GLU | C | 200 | 29.895 | 27.584 | 65.613 | 1.00 | 55.33 | C |
| ATOM | 15188 | CD | GLU | C | 200 | 28.906 | 26.930 | 66.581 | 1.00 | 53.36 | C |
| ATOM | 15189 | OE1 | GLU | C | 200 | 29.287 | 26.452 | 67.656 | 1.00 | 54.60 | O |
| ATOM | 15190 | OE2 | GLU | C | 200 | 27.691 | 26.914 | 66.248 | 1.00 | 53.86 | O |
| ATOM | 15191 | C | GLU | C | 200 | 31.860 | 29.363 | 64.350 | 1.00 | 51.65 | C |
| ATOM | 15192 | O | GLU | C | 200 | 32.292 | 30.315 | 64.973 | 1.00 | 50.64 | O |
| ATOM | 15193 | N | VAL | C | 201 | 31.026 | 29.467 | 63.307 | 1.00 | 51.45 | N |
| ATOM | 15195 | CA | VAL | C | 201 | 30.461 | 30.724 | 62.832 | 1.00 | 46.63 | C |
| ATOM | 15197 | CB | VAL | C | 201 | 31.231 | 31.274 | 61.575 | 1.00 | 46.72 | C |
| ATOM | 15199 | CG1 | VAL | C | 201 | 32.720 | 31.290 | 61.826 | 1.00 | 46.42 | C |
| ATOM | 15203 | CG2 | VAL | C | 201 | 30.925 | 30.490 | 60.319 | 1.00 | 43.26 | C |
| ATOM | 15207 | C | VAL | C | 201 | 28.940 | 30.649 | 62.528 | 1.00 | 44.99 | C |
| ATOM | 15208 | O | VAL | C | 201 | 28.376 | 29.572 | 62.414 | 1.00 | 54.23 | O |
| ATOM | 15209 | N | ILE | C | 202 | 28.293 | 31.822 | 62.411 | 1.00 | 48.31 | N |
| ATOM | 15211 | CA | ILE | C | 202 | 26.856 | 31.919 | 62.056 | 1.00 | 42.18 | C |
| ATOM | 15213 | CB | ILE | C | 202 | 25.960 | 32.584 | 63.153 | 1.00 | 46.13 | C |
| ATOM | 15215 | CG1 | ILE | C | 202 | 25.705 | 31.620 | 64.319 | 1.00 | 52.03 | C |
| ATOM | 15218 | CD1 | ILE | C | 202 | 25.448 | 32.297 | 65.708 | 1.00 | 47.21 | C |
| ATOM | 15222 | CG2 | ILE | C | 202 | 24.579 | 32.776 | 62.594 | 1.00 | 47.93 | C |
| ATOM | 15226 | C | ILE | C | 202 | 26.593 | 32.612 | 60.710 | 1.00 | 43.46 | C |
| ATOM | 15227 | O | ILE | C | 202 | 26.878 | 33.802 | 60.543 | 1.00 | 38.75 | O |
| ATOM | 15228 | N | ILE | C | 203 | 26.020 | 31.835 | 59.782 | 1.00 | 42.36 | N |
| ATOM | 15230 | CA | ILE | C | 203 | 25.653 | 32.266 | 58.452 | 1.00 | 38.42 | C |
| ATOM | 15232 | CB | ILE | C | 203 | 25.821 | 31.121 | 57.521 | 1.00 | 39.19 | C |
| ATOM | 15234 | CG1 | ILE | C | 203 | 27.291 | 30.844 | 57.270 | 1.00 | 33.90 | C |
| ATOM | 15237 | CD1 | ILE | C | 203 | 27.493 | 29.632 | 56.433 | 1.00 | 33.12 | C |
| ATOM | 15241 | CG2 | ILE | C | 203 | 25.129 | 31.425 | 56.216 | 1.00 | 43.76 | C |
| ATOM | 15245 | C | ILE | C | 203 | 24.172 | 32.651 | 58.419 | 1.00 | 46.84 | C |
| ATOM | 15246 | O | ILE | C | 203 | 23.325 | 31.774 | 58.599 | 1.00 | 42.94 | O |
| ATOM | 15247 | N | VAL | C | 204 | 23.882 | 33.938 | 58.150 | 1.00 | 44.19 | N |
| ATOM | 15249 | CA | VAL | C | 204 | 22.544 | 34.494 | 58.261 | 1.00 | 40.88 | C |
| ATOM | 15251 | CB | VAL | C | 204 | 22.551 | 35.857 | 58.956 | 1.00 | 39.68 | C |
| ATOM | 15253 | CG1 | VAL | C | 204 | 23.310 | 35.782 | 60.252 | 1.00 | 45.63 | C |
| ATOM | 15257 | CG2 | VAL | C | 204 | 23.163 | 36.889 | 58.094 | 1.00 | 38.86 | C |
| ATOM | 15261 | C | VAL | C | 204 | 21.836 | 34.673 | 56.947 | 1.00 | 41.55 | C |
| ATOM | 15262 | O | VAL | C | 204 | 20.631 | 34.867 | 56.901 | 1.00 | 40.27 | O |
| ATOM | 15263 | N | ARG | C | 205 | 22.546 | 34.630 | 55.851 | 1.00 | 44.70 | N |
| ATOM | 15265 | CA | ARG | C | 205 | 21.813 | 34.671 | 54.594 | 1.00 | 43.15 | C |
| ATOM | 15267 | CB | ARG | C | 205 | 21.363 | 36.095 | 54.387 | 1.00 | 42.20 | C |
| ATOM | 15270 | CG | ARG | C | 205 | 20.722 | 36.355 | 53.055 | 1.00 | 45.65 | C |
| ATOM | 15273 | CD | ARG | C | 205 | 20.529 | 37.833 | 52.808 | 1.00 | 40.86 | C |
| ATOM | 15276 | NE | ARG | C | 205 | 20.356 | 38.095 | 51.406 | 1.00 | 43.85 | N |
| ATOM | 15278 | CZ | ARG | C | 205 | 19.209 | 38.492 | 50.860 | 1.00 | 56.25 | C |
| ATOM | 15279 | NH1 | ARG | C | 205 | 18.138 | 38.663 | 51.623 | 1.00 | 49.46 | N |
| ATOM | 15282 | NH2 | ARG | C | 205 | 19.131 | 38.724 | 49.538 | 1.00 | 59.23 | N |
| ATOM | 15285 | C | ARG | C | 205 | 22.634 | 34.130 | 53.409 | 1.00 | 42.48 | C |
| ATOM | 15286 | O | ARG | C | 205 | 23.813 | 34.449 | 53.284 | 1.00 | 41.99 | O |
| ATOM | 15287 | N | VAL | C | 206 | 22.026 | 33.317 | 52.550 | 1.00 | 40.52 | N |
| ATOM | 15289 | CA | VAL | C | 206 | 22.737 | 32.791 | 51.379 | 1.00 | 41.02 | C |
| ATOM | 15291 | CB | VAL | C | 206 | 22.927 | 31.279 | 51.471 | 1.00 | 40.59 | C |
| ATOM | 15293 | CG1 | VAL | C | 206 | 23.317 | 30.696 | 50.129 | 1.00 | 42.15 | C |
| ATOM | 15297 | CG2 | VAL | C | 206 | 24.031 | 30.961 | 52.462 | 1.00 | 41.87 | C |
| ATOM | 15301 | C | VAL | C | 206 | 22.034 | 33.153 | 50.075 | 1.00 | 43.12 | C |
| ATOM | 15302 | O | VAL | C | 206 | 20.813 | 33.046 | 49.996 | 1.00 | 47.55 | O |
| ATOM | 15303 | N | GLU | C | 207 | 22.806 | 33.595 | 49.065 | 1.00 | 44.82 | N |
| ATOM | 15305 | CA | GLU | C | 207 | 22.279 | 34.018 | 47.753 | 1.00 | 41.44 | C |
| ATOM | 15307 | CB | GLU | C | 207 | 22.430 | 35.512 | 47.568 | 1.00 | 41.47 | C |
| ATOM | 15310 | CG | GLU | C | 207 | 21.512 | 36.430 | 48.314 | 1.00 | 42.60 | C |
| ATOM | 15313 | CD | GLU | C | 207 | 22.035 | 37.856 | 48.249 | 1.00 | 50.19 | C |
| ATOM | 15314 | OE1 | GLU | C | 207 | 22.821 | 38.160 | 47.296 | 1.00 | 52.39 | O |
| ATOM | 15315 | OE2 | GLU | C | 207 | 21.679 | 38.666 | 49.147 | 1.00 | 46.64 | O |
| ATOM | 15316 | C | GLU | C | 207 | 23.034 | 33.399 | 46.566 | 1.00 | 41.75 | C |
| ATOM | 15317 | O | GLU | C | 207 | 24.211 | 33.080 | 46.689 | 1.00 | 36.68 | O |
| ATOM | 15318 | N | ILE | C | 208 | 22.342 | 33.253 | 45.423 | 1.00 | 40.80 | N |
| ATOM | 15320 | CA | ILE | C | 208 | 22.905 | 32.702 | 44.200 | 1.00 | 40.34 | C |
| ATOM | 15322 | CB | ILE | C | 208 | 22.245 | 31.408 | 43.864 | 1.00 | 39.20 | C |
| ATOM | 15324 | CG1 | ILE | C | 208 | 22.071 | 30.557 | 45.096 | 1.00 | 40.09 | C |
| ATOM | 15327 | CD1 | ILE | C | 208 | 23.285 | 29.762 | 45.470 | 1.00 | 40.99 | C |
| ATOM | 15331 | CG2 | ILE | C | 208 | 23.075 | 30.663 | 42.809 | 1.00 | 43.38 | C |
| ATOM | 15335 | C | ILE | C | 208 | 22.593 | 33.697 | 43.111 | 1.00 | 42.32 | C |
| ATOM | 15336 | O | ILE | C | 208 | 21.440 | 33.839 | 42.734 | 1.00 | 52.77 | O |
| ATOM | 15337 | N | ASN | C | 209 | 23.622 | 34.358 | 42.581 | 1.00 | 46.89 | N |
| ATOM | 15339 | CA | ASN | C | 209 | 23.490 | 35.589 | 41.739 | 1.00 | 39.83 | C |
| ATOM | 15341 | CB | ASN | C | 209 | 23.200 | 35.302 | 40.256 | 1.00 | 34.52 | C |
| ATOM | 15344 | CG | ASN | C | 209 | 24.501 | 35.018 | 39.447 | 1.00 | 36.76 | C |
| ATOM | 15345 | OD1 | ASN | C | 209 | 25.573 | 35.001 | 40.006 | 1.00 | 41.74 | O |
| ATOM | 15346 | ND2 | ASN | C | 209 | 24.391 | 34.808 | 38.132 | 1.00 | 39.00 | N |
| ATOM | 15349 | C | ASN | C | 209 | 22.527 | 36.598 | 42.359 | 1.00 | 37.58 | C |
| ATOM | 15350 | O | ASN | C | 209 | 21.701 | 37.132 | 41.701 | 1.00 | 48.40 | O |
| ATOM | 15351 | N | GLY | C | 210 | 22.673 | 36.854 | 43.652 | 1.00 | 46.50 | N |
| ATOM | 15353 | CA | GLY | C | 210 | 21.882 | 37.848 | 44.370 | 1.00 | 41.31 | C |
| ATOM | 15356 | C | GLY | C | 210 | 20.518 | 37.398 | 44.884 | 1.00 | 41.24 | C |
| ATOM | 15357 | O | GLY | C | 210 | 19.844 | 38.161 | 45.549 | 1.00 | 37.25 | O |
| ATOM | 15358 | N | GLN | C | 211 | 20.113 | 36.163 | 44.594 | 1.00 | 46.35 | N |
| ATOM | 15360 | CA | GLN | C | 211 | 18.781 | 35.675 | 44.963 | 1.00 | 45.47 | C |
| ATOM | 15362 | CB | GLN | C | 211 | 18.262 | 34.732 | 43.882 | 1.00 | 49.01 | C |
| ATOM | 15365 | CG | GLN | C | 211 | 16.792 | 34.340 | 44.050 | 1.00 | 48.53 | C |
| ATOM | 15368 | CD | GLN | C | 211 | 16.314 | 33.366 | 42.969 | 1.00 | 52.21 | C |
| ATOM | 15369 | OE1 | GLN | C | 211 | 17.113 | 32.799 | 42.237 | 1.00 | 59.52 | O |
| ATOM | 15370 | NE2 | GLN | C | 211 | 15.007 | 33.168 | 42.882 | 1.00 | 62.94 | N |
| ATOM | 15373 | C | GLN | C | 211 | 18.784 | 34.920 | 46.237 | 1.00 | 44.55 | C |
| ATOM | 15374 | O | GLN | C | 211 | 19.593 | 34.039 | 46.396 | 1.00 | 47.96 | O |
| ATOM | 15375 | N | ASP | C | 212 | 17.866 | 35.263 | 47.137 | 1.00 | 47.82 | N |
| ATOM | 15377 | CA | ASP | C | 212 | 17.721 | 34.610 | 48.445 | 1.00 | 48.03 | C |
| ATOM | 15379 | CB | ASP | C | 212 | 16.568 | 35.289 | 49.184 | 1.00 | 46.60 | C |
| ATOM | 15382 | CG | ASP | C | 212 | 16.680 | 35.212 | 50.710 | 1.00 | 54.20 | C |
| ATOM | 15383 | OD1 | ASP | C | 212 | 17.374 | 34.336 | 51.273 | 1.00 | 61.39 | O |
| ATOM | 15384 | OD2 | ASP | C | 212 | 16.077 | 36.007 | 51.457 | 1.00 | 60.59 | O |
| ATOM | 15385 | C | ASP | C | 212 | 17.410 | 33.100 | 48.296 | 1.00 | 52.45 | C |
| ATOM | 15386 | O | ASP | C | 212 | 16.578 | 32.729 | 47.490 | 1.00 | 56.67 | O |
| ATOM | 15387 | N | LEU | C | 213 | 18.087 | 32.240 | 49.055 | 1.00 | 55.42 | N |
| ATOM | 15389 | CA | LEU | C | 213 | 17.742 | 30.820 | 49.133 | 1.00 | 56.97 | C |
| ATOM | 15391 | CB | LEU | C | 213 | 18.895 | 30.033 | 49.754 | 1.00 | 58.95 | C |
| ATOM | 15394 | CG | LEU | C | 213 | 19.416 | 28.788 | 49.047 | 1.00 | 59.16 | C |
| ATOM | 15396 | CD1 | LEU | C | 213 | 20.101 | 29.126 | 47.742 | 1.00 | 63.01 | C |
| ATOM | 15400 | CD2 | LEU | C | 213 | 20.399 | 28.127 | 49.943 | 1.00 | 60.50 | C |
| ATOM | 15404 | C | LEU | C | 213 | 16.458 | 30.806 | 50.017 | 1.00 | 62.07 | C |
| ATOM | 15405 | O | LEU | C | 213 | 15.551 | 29.957 | 49.888 | 1.00 | 61.04 | O |
| ATOM | 15406 | N | LYS | C | 214 | 16.396 | 31.769 | 50.929 | 1.00 | 63.07 | N |
| ATOM | 15408 | CA | LYS | C | 214 | 15.125 | 32.134 | 51.531 | 1.00 | 65.72 | C |
| ATOM | 15410 | CB | LYS | C | 214 | 14.086 | 32.338 | 50.413 | 1.00 | 68.54 | C |
| ATOM | 15413 | CG | LYS | C | 214 | 13.163 | 33.531 | 50.668 | 1.00 | 73.60 | C |
| ATOM | 15416 | CD | LYS | C | 214 | 11.780 | 33.375 | 50.049 | 1.00 | 77.37 | C |
| ATOM | 15419 | CE | LYS | C | 214 | 10.778 | 34.279 | 50.782 | 1.00 | 80.25 | C |
| ATOM | 15422 | NZ | LYS | C | 214 | 9.511 | 34.543 | 49.997 | 1.00 | 81.27 | N |
| ATOM | 15426 | C | LYS | C | 214 | 14.595 | 31.119 | 52.502 | 1.00 | 65.65 | C |
| ATOM | 15427 | O | LYS | C | 214 | 13.379 | 30.934 | 52.638 | 1.00 | 66.01 | O |
| ATOM | 15428 | N | MET | C | 215 | 15.509 | 30.463 | 53.188 | 1.00 | 63.58 | N |
| ATOM | 15430 | CA | MET | C | 215 | 15.135 | 29.471 | 54.158 | 1.00 | 61.94 | C |
| ATOM | 15432 | CB | MET | C | 215 | 16.070 | 28.284 | 53.990 | 1.00 | 61.98 | C |
| ATOM | 15435 | CG | MET | C | 215 | 15.962 | 27.721 | 52.571 | 1.00 | 65.30 | C |
| ATOM | 15438 | SD | MET | C | 215 | 17.057 | 26.390 | 52.158 | 1.00 | 66.94 | S |
| ATOM | 15439 | CE | MET | C | 215 | 17.630 | 26.053 | 53.654 | 1.00 | 69.99 | C |
| ATOM | 15443 | C | MET | C | 215 | 15.206 | 30.067 | 55.558 | 1.00 | 61.53 | C |
| ATOM | 15444 | O | MET | C | 215 | 15.628 | 31.208 | 55.733 | 1.00 | 62.98 | O |
| ATOM | 15445 | N | ASP | C | 216 | 14.770 | 29.312 | 56.552 | 1.00 | 59.98 | N |
| ATOM | 15447 | CA | ASP | C | 216 | 14.984 | 29.732 | 57.917 | 1.00 | 63.83 | C |
| ATOM | 15449 | CB | ASP | C | 216 | 14.421 | 28.723 | 58.910 | 1.00 | 63.59 | C |
| ATOM | 15452 | CG | ASP | C | 216 | 14.491 | 29.219 | 60.341 | 1.00 | 63.16 | C |
| ATOM | 15453 | OD1 | ASP | C | 216 | 15.548 | 29.759 | 60.739 | 1.00 | 69.24 | O |
| ATOM | 15454 | OD2 | ASP | C | 216 | 13.541 | 29.119 | 61.139 | 1.00 | 58.32 | O |
| ATOM | 15455 | C | ASP | C | 216 | 16.489 | 29.813 | 58.110 | 1.00 | 67.41 | C |
| ATOM | 15456 | O | ASP | C | 216 | 17.241 | 28.950 | 57.662 | 1.00 | 70.90 | O |
| ATOM | 15457 | N | CYS | C | 217 | 16.938 | 30.845 | 58.787 | 1.00 | 68.15 | N |
| ATOM | 15459 | CA | CYS | C | 217 | 18.351 | 31.024 | 58.969 | 1.00 | 72.70 | C |
| ATOM | 15461 | CB | CYS | C | 217 | 18.529 | 32.328 | 59.712 | 1.00 | 74.24 | C |
| ATOM | 15464 | SG | CYS | C | 217 | 18.246 | 32.242 | 61.449 | 1.00 | 86.77 | S |
| ATOM | 15465 | C | CYS | C | 217 | 19.059 | 29.819 | 59.668 | 1.00 | 71.10 | C |
| ATOM | 15466 | O | CYS | C | 217 | 20.134 | 29.351 | 59.235 | 1.00 | 66.77 | O |
| ATOM | 15467 | N | LYS | C | 218 | 18.447 | 29.314 | 60.736 | 1.00 | 70.12 | N |
| ATOM | 15469 | CA | LYS | C | 218 | 18.971 | 28.156 | 61.466 | 1.00 | 67.64 | C |
| ATOM | 15471 | CB | LYS | C | 218 | 17.922 | 27.688 | 62.483 | 1.00 | 67.20 | C |
| ATOM | 15474 | CG | LYS | C | 218 | 17.853 | 28.475 | 63.795 | 1.00 | 70.89 | C |
| ATOM | 15477 | CD | LYS | C | 218 | 17.000 | 27.722 | 64.838 | 1.00 | 73.15 | C |
| ATOM | 15480 | CE | LYS | C | 218 | 16.593 | 28.602 | 66.030 | 1.00 | 74.74 | C |
| ATOM | 15483 | NZ | LYS | C | 218 | 15.949 | 29.917 | 65.624 | 1.00 | 73.64 | N |
| ATOM | 15487 | C | LYS | C | 218 | 19.318 | 26.997 | 60.515 | 1.00 | 63.47 | C |
| ATOM | 15488 | O | LYS | C | 218 | 20.271 | 26.234 | 60.716 | 1.00 | 60.95 | O |
| ATOM | 15489 | N | GLU | C | 219 | 18.540 | 26.865 | 59.462 | 1.00 | 60.07 | N |
| ATOM | 15491 | CA | GLU | C | 219 | 18.752 | 25.751 | 58.582 | 1.00 | 60.47 | C |
| ATOM | 15493 | CB | GLU | C | 219 | 17.606 | 25.606 | 57.552 | 1.00 | 61.66 | C |
| ATOM | 15496 | CG | GLU | C | 219 | 16.924 | 24.226 | 57.549 | 1.00 | 68.73 | C |
| ATOM | 15499 | CD | GLU | C | 219 | 15.776 | 24.058 | 58.574 | 1.00 | 79.28 | C |
| ATOM | 15500 | OE1 | GLU | C | 219 | 14.678 | 24.678 | 58.418 | 1.00 | 80.66 | O |
| ATOM | 15501 | OE2 | GLU | C | 219 | 15.962 | 23.280 | 59.549 | 1.00 | 81.82 | O |
| ATOM | 15502 | C | GLU | C | 219 | 20.130 | 25.899 | 57.963 | 1.00 | 56.72 | C |
| ATOM | 15503 | O | GLU | C | 219 | 20.748 | 24.903 | 57.588 | 1.00 | 59.58 | O |
| ATOM | 15504 | N | TYR | C | 220 | 20.643 | 27.126 | 57.870 | 1.00 | 56.94 | N |
| ATOM | 15506 | CA | TYR | C | 220 | 22.003 | 27.337 | 57.313 | 1.00 | 51.25 | C |
| ATOM | 15508 | CB | TYR | C | 220 | 22.231 | 28.803 | 56.908 | 1.00 | 50.12 | C |
| ATOM | 15511 | CG | TYR | C | 220 | 21.253 | 29.350 | 55.891 | 1.00 | 51.08 | C |
| ATOM | 15512 | CD1 | TYR | C | 220 | 20.657 | 30.589 | 56.072 | 1.00 | 52.25 | C |
| ATOM | 15514 | CE1 | TYR | C | 220 | 19.758 | 31.101 | 55.142 | 1.00 | 50.67 | C |
| ATOM | 15516 | CZ | TYR | C | 220 | 19.460 | 30.362 | 54.028 | 1.00 | 48.80 | C |
| ATOM | 15517 | OH | TYR | C | 220 | 18.584 | 30.861 | 53.123 | 1.00 | 54.70 | O |
| ATOM | 15519 | CE2 | TYR | C | 220 | 20.029 | 29.140 | 53.811 | 1.00 | 47.32 | C |
| ATOM | 15521 | CD2 | TYR | C | 220 | 20.927 | 28.634 | 54.741 | 1.00 | 53.35 | C |
| ATOM | 15523 | C | TYR | C | 220 | 23.106 | 26.890 | 58.295 | 1.00 | 50.69 | C |
| ATOM | 15524 | O | TYR | C | 220 | 24.252 | 26.689 | 57.913 | 1.00 | 49.00 | O |
| ATOM | 15525 | N | ASN | C | 221 | 22.780 | 26.754 | 59.569 | 1.00 | 50.57 | N |
| ATOM | 15527 | CA | ASN | C | 221 | 23.770 | 26.262 | 60.506 | 1.00 | 55.15 | C |
| ATOM | 15529 | CB | ASN | C | 221 | 24.208 | 27.421 | 61.384 | 1.00 | 55.72 | C |
| ATOM | 15532 | CG | ASN | C | 221 | 24.244 | 28.687 | 60.624 | 1.00 | 58.30 | C |
| ATOM | 15533 | OD1 | ASN | C | 221 | 25.274 | 29.062 | 60.045 | 1.00 | 61.72 | O |
| ATOM | 15534 | ND2 | ASN | C | 221 | 23.104 | 29.357 | 60.582 | 1.00 | 60.35 | N |
| ATOM | 15537 | C | ASN | C | 221 | 23.219 | 25.081 | 61.310 | 1.00 | 55.63 | C |
| ATOM | 15538 | O | ASN | C | 221 | 23.444 | 24.978 | 62.517 | 1.00 | 53.22 | O |
| ATOM | 15539 | N | TYR | C | 222 | 22.510 | 24.188 | 60.627 | 1.00 | 55.35 | N |
| ATOM | 15541 | CA | TYR | C | 222 | 21.764 | 23.173 | 61.334 | 1.00 | 55.70 | C |
| ATOM | 15543 | CB | TYR | C | 222 | 20.643 | 22.579 | 60.521 | 1.00 | 59.55 | C |
| ATOM | 15546 | CG | TYR | C | 222 | 20.139 | 21.358 | 61.212 | 1.00 | 63.51 | C |
| ATOM | 15547 | CD1 | TYR | C | 222 | 19.875 | 21.375 | 62.578 | 1.00 | 65.98 | C |
| ATOM | 15549 | CE1 | TYR | C | 222 | 19.414 | 20.235 | 63.230 | 1.00 | 69.85 | C |
| ATOM | 15551 | CZ | TYR | C | 222 | 19.215 | 19.049 | 62.511 | 1.00 | 72.74 | C |
| ATOM | 15552 | OH | TYR | C | 222 | 18.757 | 17.898 | 63.130 | 1.00 | 71.88 | O |
| ATOM | 15554 | CE2 | TYR | C | 222 | 19.476 | 19.019 | 61.155 | 1.00 | 72.46 | C |
| ATOM | 15556 | CD2 | TYR | C | 222 | 19.940 | 20.174 | 60.515 | 1.00 | 66.13 | C |
| ATOM | 15558 | C | TYR | C | 222 | 22.748 | 22.140 | 61.714 | 1.00 | 55.15 | C |
| ATOM | 15559 | O | TYR | C | 222 | 23.304 | 21.468 | 60.853 | 1.00 | 51.38 | O |
| ATOM | 15560 | N | ASP | C | 223 | 22.904 | 22.013 | 63.027 | 1.00 | 60.19 | N |
| ATOM | 15562 | CA | ASP | C | 223 | 24.044 | 21.398 | 63.668 | 1.00 | 57.35 | C |
| ATOM | 15564 | CB | ASP | C | 223 | 24.600 | 20.222 | 62.885 | 1.00 | 64.20 | C |
| ATOM | 15567 | CG | ASP | C | 223 | 25.633 | 19.430 | 63.682 | 1.00 | 68.05 | C |
| ATOM | 15568 | OD1 | ASP | C | 223 | 26.491 | 18.782 | 63.058 | 1.00 | 75.23 | O |
| ATOM | 15569 | OD2 | ASP | C | 223 | 25.669 | 19.410 | 64.935 | 1.00 | 65.90 | O |
| ATOM | 15570 | C | ASP | C | 223 | 25.109 | 22.491 | 63.857 | 1.00 | 53.89 | C |
| ATOM | 15571 | O | ASP | C | 223 | 25.364 | 22.902 | 64.969 | 1.00 | 51.49 | O |
| ATOM | 15572 | N | LYS | C | 224 | 25.729 | 22.980 | 62.788 | 1.00 | 51.65 | N |
| ATOM | 15574 | CA | LYS | C | 224 | 26.750 | 24.020 | 62.949 | 1.00 | 48.28 | C |
| ATOM | 15576 | CB | LYS | C | 224 | 27.919 | 23.538 | 63.825 | 1.00 | 49.33 | C |
| ATOM | 15579 | CG | LYS | C | 224 | 28.786 | 22.456 | 63.125 | 1.00 | 50.13 | C |
| ATOM | 15582 | CD | LYS | C | 224 | 29.770 | 21.673 | 64.049 | 1.00 | 51.00 | C |
| ATOM | 15585 | CE | LYS | C | 224 | 29.958 | 20.230 | 63.586 | 1.00 | 56.39 | C |
| ATOM | 15588 | NZ | LYS | C | 224 | 28.761 | 19.360 | 63.812 | 1.00 | 54.03 | N |
| ATOM | 15592 | C | LYS | C | 224 | 27.355 | 24.489 | 61.644 | 1.00 | 50.04 | C |
| ATOM | 15593 | O | LYS | C | 224 | 27.279 | 23.799 | 60.619 | 1.00 | 44.69 | O |
| ATOM | 15594 | N | SER | C | 225 | 27.960 | 25.684 | 61.708 | 1.00 | 50.25 | N |
| ATOM | 15596 | CA | SER | C | 225 | 28.747 | 26.210 | 60.604 | 1.00 | 47.90 | C |
| ATOM | 15598 | CB | SER | C | 225 | 28.098 | 27.468 | 60.076 | 1.00 | 47.14 | C |
| ATOM | 15601 | OG | SER | C | 225 | 26.865 | 27.124 | 59.460 | 1.00 | 52.47 | O |
| ATOM | 15603 | C | SER | C | 225 | 30.179 | 26.415 | 61.102 | 1.00 | 44.94 | C |
| ATOM | 15604 | O | SER | C | 225 | 30.396 | 27.152 | 62.055 | 1.00 | 47.22 | O |
| ATOM | 15605 | N | ILE | C | 226 | 31.149 | 25.745 | 60.479 | 1.00 | 45.19 | N |
| ATOM | 15607 | CA | ILE | C | 226 | 32.557 | 25.868 | 60.888 | 1.00 | 43.93 | C |
| ATOM | 15609 | CB | ILE | C | 226 | 33.045 | 24.557 | 61.593 | 1.00 | 39.91 | C |
| ATOM | 15611 | CG1 | ILE | C | 226 | 33.415 | 23.500 | 60.579 | 1.00 | 39.64 | C |
| ATOM | 15614 | CD1 | ILE | C | 226 | 34.031 | 22.226 | 61.231 | 1.00 | 37.78 | C |
| ATOM | 15618 | CG2 | ILE | C | 226 | 31.999 | 24.023 | 62.556 | 1.00 | 42.23 | C |
| ATOM | 15622 | C | ILE | C | 226 | 33.472 | 26.181 | 59.710 | 1.00 | 43.36 | C |
| ATOM | 15623 | O | ILE | C | 226 | 33.060 | 26.084 | 58.545 | 1.00 | 42.72 | O |
| ATOM | 15624 | N | VAL | C | 227 | 34.717 | 26.544 | 60.020 | 1.00 | 45.45 | N |
| ATOM | 15626 | CA | VAL | C | 227 | 35.743 | 26.881 | 59.000 | 1.00 | 48.25 | C |
| ATOM | 15628 | CB | VAL | C | 227 | 36.309 | 28.367 | 59.169 | 1.00 | 47.46 | C |
| ATOM | 15630 | CG1 | VAL | C | 227 | 37.483 | 28.629 | 58.234 | 1.00 | 46.44 | C |
| ATOM | 15634 | CG2 | VAL | C | 227 | 35.241 | 29.399 | 58.940 | 1.00 | 48.83 | C |
| ATOM | 15638 | C | VAL | C | 227 | 36.907 | 25.894 | 59.111 | 1.00 | 44.62 | C |
| ATOM | 15639 | O | VAL | C | 227 | 37.588 | 25.862 | 60.143 | 1.00 | 41.47 | O |
| ATOM | 15640 | N | ASP | C | 228 | 37.146 | 25.120 | 58.043 | 1.00 | 45.60 | N |
| ATOM | 15642 | CA | ASP | C | 228 | 38.103 | 24.013 | 58.065 | 1.00 | 43.43 | C |
| ATOM | 15644 | CB | ASP | C | 228 | 37.291 | 22.734 | 58.217 | 1.00 | 44.61 | C |
| ATOM | 15647 | CG | ASP | C | 228 | 38.162 | 21.501 | 58.399 | 1.00 | 49.06 | C |
| ATOM | 15648 | OD1 | ASP | C | 228 | 39.346 | 21.674 | 58.748 | 1.00 | 51.29 | O |
| ATOM | 15649 | OD2 | ASP | C | 228 | 37.754 | 20.325 | 58.225 | 1.00 | 48.80 | O |
| ATOM | 15650 | C | ASP | C | 228 | 39.059 | 23.867 | 56.868 | 1.00 | 42.97 | C |
| ATOM | 15651 | O | ASP | C | 228 | 38.667 | 23.383 | 55.830 | 1.00 | 51.06 | O |
| ATOM | 15652 | N | SER | C | 229 | 40.319 | 24.264 | 57.035 | 1.00 | 42.95 | N |
| ATOM | 15654 | CA | SER | C | 229 | 41.355 | 24.153 | 55.999 | 1.00 | 42.72 | C |
| ATOM | 15656 | CB | SER | C | 229 | 42.649 | 24.816 | 56.493 | 1.00 | 45.39 | C |
| ATOM | 15659 | OG | SER | C | 229 | 43.168 | 24.142 | 57.661 | 1.00 | 48.69 | O |
| ATOM | 15661 | C | SER | C | 229 | 41.719 | 22.733 | 55.619 | 1.00 | 43.75 | C |
| ATOM | 15662 | O | SER | C | 229 | 42.444 | 22.509 | 54.667 | 1.00 | 44.82 | O |
| ATOM | 15663 | N | GLY | C | 230 | 41.256 | 21.765 | 56.389 | 1.00 | 47.33 | N |
| ATOM | 15665 | CA | GLY | C | 230 | 41.472 | 20.366 | 56.072 | 1.00 | 45.82 | C |
| ATOM | 15668 | C | GLY | C | 230 | 40.324 | 19.725 | 55.294 | 1.00 | 47.37 | C |
| ATOM | 15669 | O | GLY | C | 230 | 40.365 | 18.517 | 55.074 | 1.00 | 50.08 | O |
| ATOM | 15670 | N | THR | C | 231 | 39.329 | 20.528 | 54.875 | 1.00 | 50.13 | N |
| ATOM | 15672 | CA | THR | C | 231 | 38.127 | 20.075 | 54.126 | 1.00 | 44.81 | C |
| ATOM | 15674 | CB | THR | C | 231 | 36.847 | 20.427 | 54.846 | 1.00 | 41.41 | C |
| ATOM | 15676 | OG1 | THR | C | 231 | 36.874 | 19.913 | 56.171 | 1.00 | 42.47 | O |
| ATOM | 15678 | CG2 | THR | C | 231 | 35.737 | 19.674 | 54.262 | 1.00 | 45.87 | C |
| ATOM | 15682 | C | THR | C | 231 | 38.097 | 20.737 | 52.773 | 1.00 | 46.97 | C |
| ATOM | 15683 | O | THR | C | 231 | 38.225 | 21.973 | 52.653 | 1.00 | 41.58 | O |
| ATOM | 15684 | N | THR | C | 232 | 37.903 | 19.909 | 51.755 | 1.00 | 47.97 | N |
| ATOM | 15686 | CA | THR | C | 232 | 38.114 | 20.336 | 50.382 | 1.00 | 48.47 | C |
| ATOM | 15688 | CB | THR | C | 232 | 38.315 | 19.108 | 49.509 | 1.00 | 47.83 | C |
| ATOM | 15690 | OG1 | THR | C | 232 | 39.514 | 18.422 | 49.871 | 1.00 | 46.26 | O |
| ATOM | 15692 | CG2 | THR | C | 232 | 38.589 | 19.543 | 48.087 | 1.00 | 52.26 | C |
| ATOM | 15696 | C | THR | C | 232 | 36.991 | 21.136 | 49.741 | 1.00 | 49.88 | C |
| ATOM | 15697 | O | THR | C | 232 | 37.264 | 22.026 | 48.965 | 1.00 | 52.26 | O |
| ATOM | 15698 | N | ASN | C | 233 | 35.743 | 20.788 | 50.054 | 1.00 | 47.73 | N |
| ATOM | 15700 | CA | ASN | C | 233 | 34.586 | 21.318 | 49.385 | 1.00 | 44.90 | C |
| ATOM | 15702 | CB | ASN | C | 233 | 33.570 | 20.208 | 49.063 | 1.00 | 44.37 | C |
| ATOM | 15705 | CG | ASN | C | 233 | 34.025 | 19.181 | 48.006 | 1.00 | 47.43 | C |
| ATOM | 15706 | OD1 | ASN | C | 233 | 35.051 | 19.285 | 47.345 | 1.00 | 52.27 | O |
| ATOM | 15707 | ND2 | ASN | C | 233 | 33.216 | 18.155 | 47.871 | 1.00 | 47.91 | N |
| ATOM | 15710 | C | ASN | C | 233 | 33.865 | 22.287 | 50.313 | 1.00 | 49.52 | C |
| ATOM | 15711 | O | ASN | C | 233 | 34.283 | 22.502 | 51.451 | 1.00 | 51.70 | O |
| ATOM | 15712 | N | LEU | C | 234 | 32.787 | 22.878 | 49.792 | 1.00 | 49.50 | N |
| ATOM | 15714 | CA | LEU | C | 234 | 31.851 | 23.671 | 50.561 | 1.00 | 50.43 | C |
| ATOM | 15716 | CB | LEU | C | 234 | 31.168 | 24.715 | 49.667 | 1.00 | 49.25 | C |
| ATOM | 15719 | CG | LEU | C | 234 | 30.577 | 25.984 | 50.268 | 1.00 | 46.33 | C |
| ATOM | 15721 | CD1 | LEU | C | 234 | 29.479 | 26.523 | 49.377 | 1.00 | 40.51 | C |
| ATOM | 15725 | CD2 | LEU | C | 234 | 30.090 | 25.777 | 51.667 | 1.00 | 46.97 | C |
| ATOM | 15729 | C | LEU | C | 234 | 30.852 | 22.593 | 50.965 | 1.00 | 49.62 | C |
| ATOM | 15730 | O | LEU | C | 234 | 30.407 | 21.816 | 50.116 | 1.00 | 45.22 | O |
| ATOM | 15731 | N | ARG | C | 235 | 30.491 | 22.520 | 52.239 | 1.00 | 48.90 | N |
| ATOM | 15733 | CA | ARG | C | 235 | 29.625 | 21.424 | 52.677 | 1.00 | 44.81 | C |
| ATOM | 15735 | CB | ARG | C | 235 | 30.348 | 20.503 | 53.656 | 1.00 | 43.27 | C |
| ATOM | 15738 | CG | ARG | C | 235 | 31.341 | 19.584 | 53.002 | 1.00 | 43.89 | C |
| ATOM | 15741 | CD | ARG | C | 235 | 30.842 | 18.160 | 52.801 | 1.00 | 50.43 | C |
| ATOM | 15744 | NE | ARG | C | 235 | 31.337 | 17.255 | 53.837 | 1.00 | 50.04 | N |
| ATOM | 15746 | CZ | ARG | C | 235 | 31.221 | 15.938 | 53.811 | 1.00 | 45.48 | C |
| ATOM | 15747 | NH1 | ARG | C | 235 | 31.708 | 15.258 | 54.800 | 1.00 | 46.80 | N |
| ATOM | 15750 | NH2 | ARG | C | 235 | 30.623 | 15.297 | 52.831 | 1.00 | 47.49 | N |
| ATOM | 15753 | C | ARG | C | 235 | 28.396 | 21.986 | 53.309 | 1.00 | 40.98 | C |
| ATOM | 15754 | O | ARG | C | 235 | 28.468 | 22.809 | 54.227 | 1.00 | 37.83 | O |
| ATOM | 15755 | N | LEU | C | 236 | 27.250 | 21.535 | 52.823 | 1.00 | 39.74 | N |
| ATOM | 15757 | CA | LEU | C | 236 | 26.029 | 22.121 | 53.291 | 1.00 | 40.54 | C |
| ATOM | 15759 | CB | LEU | C | 236 | 25.356 | 23.023 | 52.230 | 1.00 | 44.87 | C |
| ATOM | 15762 | CG | LEU | C | 236 | 26.128 | 24.202 | 51.585 | 1.00 | 44.63 | C |
| ATOM | 15764 | CD1 | LEU | C | 236 | 26.000 | 24.076 | 50.073 | 1.00 | 44.76 | C |
| ATOM | 15768 | CD2 | LEU | C | 236 | 25.626 | 25.572 | 52.036 | 1.00 | 45.65 | C |
| ATOM | 15772 | C | LEU | C | 236 | 25.108 | 21.049 | 53.643 | 1.00 | 43.06 | C |
| ATOM | 15773 | O | LEU | C | 236 | 24.992 | 20.025 | 52.998 | 1.00 | 50.00 | O |
| ATOM | 15774 | N | PRO | C | 237 | 24.421 | 21.296 | 54.709 | 1.00 | 43.05 | N |
| ATOM | 15775 | CA | PRO | C | 237 | 23.369 | 20.404 | 55.125 | 1.00 | 47.30 | C |
| ATOM | 15777 | CB | PRO | C | 237 | 22.620 | 21.271 | 56.095 | 1.00 | 43.42 | C |
| ATOM | 15780 | CG | PRO | C | 237 | 23.724 | 22.119 | 56.697 | 1.00 | 39.72 | C |
| ATOM | 15783 | CD | PRO | C | 237 | 24.594 | 22.448 | 55.598 | 1.00 | 39.54 | C |
| ATOM | 15786 | C | PRO | C | 237 | 22.468 | 19.981 | 53.957 | 1.00 | 56.07 | C |
| ATOM | 15787 | O | PRO | C | 237 | 22.182 | 20.767 | 53.053 | 1.00 | 61.19 | O |
| ATOM | 15788 | N | LYS | C | 238 | 22.049 | 18.724 | 54.011 | 1.00 | 64.48 | N |
| ATOM | 15790 | CA | LYS | C | 238 | 21.060 | 18.088 | 53.117 | 1.00 | 67.74 | C |
| ATOM | 15792 | CB | LYS | C | 238 | 20.344 | 16.981 | 53.939 | 1.00 | 70.89 | C |
| ATOM | 15795 | CG | LYS | C | 238 | 19.364 | 16.043 | 53.183 | 1.00 | 75.72 | C |
| ATOM | 15798 | CD | LYS | C | 238 | 20.022 | 15.198 | 52.057 | 1.00 | 77.64 | C |
| ATOM | 15801 | CE | LYS | C | 238 | 19.238 | 13.898 | 51.786 | 1.00 | 76.46 | C |
| ATOM | 15804 | NZ | LYS | C | 238 | 19.681 | 13.117 | 50.588 | 1.00 | 74.66 | N |
| ATOM | 15808 | C | LYS | C | 238 | 20.005 | 19.015 | 52.472 | 1.00 | 67.39 | C |
| ATOM | 15809 | O | LYS | C | 238 | 19.814 | 19.005 | 51.247 | 1.00 | 67.71 | O |
| ATOM | 15810 | N | LYS | C | 239 | 19.303 | 19.801 | 53.277 | 1.00 | 64.13 | N |
| ATOM | 15812 | CA | LYS | C | 239 | 18.305 | 20.683 | 52.716 | 1.00 | 64.27 | C |
| ATOM | 15814 | CB | LYS | C | 239 | 17.338 | 21.171 | 53.792 | 1.00 | 68.69 | C |
| ATOM | 15817 | CG | LYS | C | 239 | 16.086 | 20.292 | 53.968 | 1.00 | 74.28 | C |
| ATOM | 15820 | CD | LYS | C | 239 | 15.063 | 20.977 | 54.894 | 1.00 | 76.92 | C |
| ATOM | 15823 | CE | LYS | C | 239 | 14.640 | 22.366 | 54.381 | 1.00 | 76.03 | C |
| ATOM | 15826 | NZ | LYS | C | 239 | 13.915 | 23.177 | 55.415 | 1.00 | 74.75 | N |
| ATOM | 15830 | C | LYS | C | 239 | 18.918 | 21.886 | 52.013 | 1.00 | 62.66 | C |
| ATOM | 15831 | O | LYS | C | 239 | 18.495 | 22.234 | 50.903 | 1.00 | 62.29 | O |
| ATOM | 15832 | N | VAL | C | 240 | 19.913 | 22.528 | 52.637 | 1.00 | 56.54 | N |
| ATOM | 15834 | CA | VAL | C | 240 | 20.450 | 23.737 | 52.039 | 1.00 | 48.20 | C |
| ATOM | 15836 | CB | VAL | C | 240 | 21.459 | 24.457 | 52.876 | 1.00 | 47.96 | C |
| ATOM | 15838 | CG1 | VAL | C | 240 | 21.799 | 25.770 | 52.198 | 1.00 | 51.82 | C |
| ATOM | 15842 | CG2 | VAL | C | 240 | 20.934 | 24.744 | 54.268 | 1.00 | 49.07 | C |
| ATOM | 15846 | C | VAL | C | 240 | 21.110 | 23.315 | 50.780 | 1.00 | 44.11 | C |
| ATOM | 15847 | O | VAL | C | 240 | 21.003 | 23.955 | 49.756 | 1.00 | 40.57 | O |
| ATOM | 15848 | N | PHE | C | 241 | 21.797 | 22.200 | 50.839 | 1.00 | 43.45 | N |
| ATOM | 15850 | CA | PHE | C | 241 | 22.457 | 21.728 | 49.645 | 1.00 | 44.85 | C |
| ATOM | 15852 | CB | PHE | C | 241 | 23.107 | 20.393 | 49.912 | 1.00 | 44.07 | C |
| ATOM | 15855 | CG | PHE | C | 241 | 23.738 | 19.768 | 48.699 | 1.00 | 44.31 | C |
| ATOM | 15856 | CD1 | PHE | C | 241 | 25.015 | 20.034 | 48.369 | 1.00 | 40.53 | C |
| ATOM | 15858 | CE1 | PHE | C | 241 | 25.575 | 19.460 | 47.272 | 1.00 | 42.85 | C |
| ATOM | 15860 | CZ | PHE | C | 241 | 24.879 | 18.608 | 46.499 | 1.00 | 35.50 | C |
| ATOM | 15862 | CE2 | PHE | C | 241 | 23.628 | 18.323 | 46.804 | 1.00 | 37.83 | C |
| ATOM | 15864 | CD2 | PHE | C | 241 | 23.039 | 18.897 | 47.899 | 1.00 | 44.17 | C |
| ATOM | 15866 | C | PHE | C | 241 | 21.495 | 21.587 | 48.488 | 1.00 | 46.73 | C |
| ATOM | 15867 | O | PHE | C | 241 | 21.880 | 21.762 | 47.328 | 1.00 | 46.03 | O |
| ATOM | 15868 | N | GLU | C | 242 | 20.241 | 21.294 | 48.824 | 1.00 | 50.74 | N |
| ATOM | 15870 | CA | GLU | C | 242 | 19.228 | 20.899 | 47.851 | 1.00 | 51.33 | C |
| ATOM | 15872 | CB | GLU | C | 242 | 18.100 | 20.168 | 48.570 | 1.00 | 56.02 | C |
| ATOM | 15875 | CG | GLU | C | 242 | 18.214 | 18.657 | 48.515 | 1.00 | 62.01 | C |
| ATOM | 15878 | CD | GLU | C | 242 | 18.576 | 18.183 | 47.122 | 1.00 | 66.03 | C |
| ATOM | 15879 | OE1 | GLU | C | 242 | 18.159 | 18.881 | 46.155 | 1.00 | 67.53 | O |
| ATOM | 15880 | OE2 | GLU | C | 242 | 19.277 | 17.134 | 46.997 | 1.00 | 60.97 | O |
| ATOM | 15881 | C | GLU | C | 242 | 18.645 | 22.076 | 47.116 | 1.00 | 50.90 | C |
| ATOM | 15882 | O | GLU | C | 242 | 18.470 | 22.057 | 45.901 | 1.00 | 46.64 | O |
| ATOM | 15883 | N | ALA | C | 243 | 18.337 | 23.100 | 47.883 | 1.00 | 47.93 | N |
| ATOM | 15885 | CA | ALA | C | 243 | 17.831 | 24.310 | 47.324 | 1.00 | 50.32 | C |
| ATOM | 15887 | CB | ALA | C | 243 | 17.431 | 25.228 | 48.429 | 1.00 | 49.97 | C |
| ATOM | 15891 | C | ALA | C | 243 | 18.932 | 24.932 | 46.484 | 1.00 | 53.72 | C |
| ATOM | 15892 | O | ALA | C | 243 | 18.694 | 25.406 | 45.376 | 1.00 | 58.27 | O |
| ATOM | 15893 | N | ALA | C | 244 | 20.142 | 24.925 | 47.020 | 1.00 | 55.49 | N |
| ATOM | 15895 | CA | ALA | C | 244 | 21.270 | 25.524 | 46.335 | 1.00 | 53.08 | C |
| ATOM | 15897 | CB | ALA | C | 244 | 22.505 | 25.573 | 47.229 | 1.00 | 55.11 | C |
| ATOM | 15901 | C | ALA | C | 244 | 21.575 | 24.821 | 45.057 | 1.00 | 53.02 | C |
| ATOM | 15902 | O | ALA | C | 244 | 21.779 | 25.481 | 44.073 | 1.00 | 60.49 | O |
| ATOM | 15903 | N | VAL | C | 245 | 21.608 | 23.495 | 45.025 | 1.00 | 56.92 | N |
| ATOM | 15905 | CA | VAL | C | 245 | 21.907 | 22.825 | 43.748 | 1.00 | 53.60 | C |
| ATOM | 15907 | CB | VAL | C | 245 | 22.025 | 21.300 | 43.842 | 1.00 | 55.63 | C |
| ATOM | 15909 | CG1 | VAL | C | 245 | 22.667 | 20.709 | 42.554 | 1.00 | 54.38 | C |
| ATOM | 15913 | CG2 | VAL | C | 245 | 22.819 | 20.900 | 45.014 | 1.00 | 58.96 | C |
| ATOM | 15917 | C | VAL | C | 245 | 20.804 | 23.069 | 42.734 | 1.00 | 55.12 | C |
| ATOM | 15918 | O | VAL | C | 245 | 21.060 | 22.974 | 41.544 | 1.00 | 58.24 | O |
| ATOM | 15919 | N | LYS | C | 246 | 19.574 | 23.356 | 43.160 | 1.00 | 52.47 | N |
| ATOM | 15921 | CA | LYS | C | 246 | 18.549 | 23.620 | 42.157 | 1.00 | 58.91 | C |
| ATOM | 15923 | CB | LYS | C | 246 | 17.129 | 23.817 | 42.754 | 1.00 | 62.86 | C |
| ATOM | 15926 | CG | LYS | C | 246 | 16.324 | 22.600 | 43.183 | 1.00 | 70.41 | C |
| ATOM | 15929 | CD | LYS | C | 246 | 16.388 | 21.363 | 42.223 | 1.00 | 76.82 | C |
| ATOM | 15932 | CE | LYS | C | 246 | 17.595 | 20.433 | 42.499 | 1.00 | 77.01 | C |
| ATOM | 15935 | NZ | LYS | C | 246 | 17.683 | 19.311 | 41.510 | 1.00 | 75.94 | N |
| ATOM | 15939 | C | LYS | C | 246 | 18.944 | 24.940 | 41.486 | 1.00 | 56.47 | C |
| ATOM | 15940 | O | LYS | C | 246 | 19.066 | 25.062 | 40.257 | 1.00 | 59.03 | O |
| ATOM | 15941 | N | SER | C | 247 | 19.153 | 25.920 | 42.348 | 1.00 | 47.24 | N |
| ATOM | 15943 | CA | SER | C | 247 | 19.348 | 27.272 | 41.934 | 1.00 | 47.24 | C |
| ATOM | 15945 | CB | SER | C | 247 | 19.509 | 28.132 | 43.169 | 1.00 | 47.72 | C |
| ATOM | 15948 | OG | SER | C | 247 | 19.430 | 29.494 | 42.810 | 1.00 | 53.48 | O |
| ATOM | 15950 | C | SER | C | 247 | 20.563 | 27.421 | 41.042 | 1.00 | 47.61 | C |
| ATOM | 15951 | O | SER | C | 247 | 20.552 | 28.143 | 40.030 | 1.00 | 48.26 | O |
| ATOM | 15952 | N | ILE | C | 248 | 21.612 | 26.730 | 41.432 | 1.00 | 40.55 | N |
| ATOM | 15954 | CA | ILE | C | 248 | 22.802 | 26.795 | 40.710 | 1.00 | 39.34 | C |
| ATOM | 15956 | CB | ILE | C | 248 | 23.804 | 25.942 | 41.355 | 1.00 | 39.28 | C |
| ATOM | 15958 | CG1 | ILE | C | 248 | 24.164 | 26.530 | 42.675 | 1.00 | 37.69 | C |
| ATOM | 15961 | CD1 | ILE | C | 248 | 25.595 | 26.384 | 42.926 | 1.00 | 40.41 | C |
| ATOM | 15965 | CG2 | ILE | C | 248 | 25.073 | 25.940 | 40.565 | 1.00 | 45.18 | C |
| ATOM | 15969 | C | ILE | C | 248 | 22.498 | 26.311 | 39.348 | 1.00 | 47.60 | C |
| ATOM | 15970 | O | ILE | C | 248 | 22.793 | 27.013 | 38.398 | 1.00 | 52.36 | O |
| ATOM | 15971 | N | LYS | C | 249 | 21.894 | 25.118 | 39.244 | 1.00 | 54.81 | N |
| ATOM | 15973 | CA | LYS | C | 249 | 21.619 | 24.488 | 37.939 | 1.00 | 52.55 | C |
| ATOM | 15975 | CB | LYS | C | 249 | 20.735 | 23.263 | 38.079 | 1.00 | 55.84 | C |
| ATOM | 15978 | CG | LYS | C | 249 | 21.397 | 21.980 | 38.542 | 1.00 | 60.75 | C |
| ATOM | 15981 | CD | LYS | C | 249 | 20.426 | 20.783 | 38.337 | 1.00 | 63.48 | C |
| ATOM | 15984 | CE | LYS | C | 249 | 20.792 | 19.557 | 39.172 | 1.00 | 67.61 | C |
| ATOM | 15987 | NZ | LYS | C | 249 | 19.732 | 19.211 | 40.190 | 1.00 | 73.03 | N |
| ATOM | 15991 | C | LYS | C | 249 | 20.837 | 25.463 | 37.110 | 1.00 | 51.86 | C |
| ATOM | 15992 | O | LYS | C | 249 | 21.209 | 25.796 | 35.990 | 1.00 | 56.70 | O |
| ATOM | 15993 | N | ALA | C | 250 | 19.737 | 25.924 | 37.683 | 1.00 | 44.72 | N |
| ATOM | 15995 | CA | ALA | C | 250 | 18.871 | 26.855 | 36.999 | 1.00 | 43.29 | C |
| ATOM | 15997 | CB | ALA | C | 250 | 17.850 | 27.442 | 37.977 | 1.00 | 38.29 | C |
| ATOM | 16001 | C | ALA | C | 250 | 19.696 | 27.950 | 36.371 | 1.00 | 45.27 | C |
| ATOM | 16002 | O | ALA | C | 250 | 19.535 | 28.253 | 35.199 | 1.00 | 46.37 | O |
| ATOM | 16003 | N | ALA | C | 251 | 20.600 | 28.539 | 37.153 | 1.00 | 44.64 | N |
| ATOM | 16005 | CA | ALA | C | 251 | 21.362 | 29.667 | 36.658 | 1.00 | 42.02 | C |
| ATOM | 16007 | CB | ALA | C | 251 | 22.173 | 30.242 | 37.739 | 1.00 | 39.56 | C |
| ATOM | 16011 | C | ALA | C | 251 | 22.237 | 29.272 | 35.492 | 1.00 | 44.55 | C |
| ATOM | 16012 | O | ALA | C | 251 | 22.459 | 30.029 | 34.574 | 1.00 | 53.96 | O |
| ATOM | 16013 | N | SER | C | 252 | 22.716 | 28.059 | 35.523 | 1.00 | 48.25 | N |
| ATOM | 16015 | CA | SER | C | 252 | 23.581 | 27.567 | 34.487 | 1.00 | 50.72 | C |
| ATOM | 16017 | CB | SER | C | 252 | 24.561 | 26.537 | 35.095 | 1.00 | 50.46 | C |
| ATOM | 16020 | OG | SER | C | 252 | 24.087 | 26.053 | 36.372 | 1.00 | 50.66 | O |
| ATOM | 16022 | C | SER | C | 252 | 22.746 | 26.890 | 33.427 | 1.00 | 51.45 | C |
| ATOM | 16023 | O | SER | C | 252 | 23.305 | 26.186 | 32.595 | 1.00 | 54.49 | O |
| ATOM | 16024 | N | SER | C | 253 | 21.428 | 27.104 | 33.448 | 1.00 | 52.44 | N |
| ATOM | 16026 | CA | SER | C | 253 | 20.502 | 26.337 | 32.588 | 1.00 | 55.69 | C |
| ATOM | 16028 | CB | SER | C | 253 | 19.025 | 26.589 | 32.995 | 1.00 | 58.26 | C |
| ATOM | 16031 | OG | SER | C | 253 | 18.469 | 27.826 | 32.540 | 1.00 | 55.32 | O |
| ATOM | 16033 | C | SER | C | 253 | 20.673 | 26.458 | 31.073 | 1.00 | 58.55 | C |
| ATOM | 16034 | O | SER | C | 253 | 19.693 | 26.428 | 30.343 | 1.00 | 67.47 | O |
| ATOM | 16035 | N | THR | C | 254 | 21.917 | 26.600 | 30.614 | 1.00 | 62.21 | N |
| ATOM | 16037 | CA | THR | C | 254 | 22.258 | 26.667 | 29.181 | 1.00 | 64.73 | C |
| ATOM | 16039 | CB | THR | C | 254 | 22.914 | 27.939 | 28.896 | 1.00 | 66.84 | C |
| ATOM | 16041 | OG1 | THR | C | 254 | 24.215 | 27.899 | 29.500 | 1.00 | 71.03 | O |
| ATOM | 16043 | CG2 | THR | C | 254 | 22.183 | 29.063 | 29.569 | 1.00 | 72.72 | C |
| ATOM | 16047 | C | THR | C | 254 | 23.294 | 25.658 | 28.754 | 1.00 | 68.84 | C |
| ATOM | 16048 | O | THR | C | 254 | 23.746 | 25.640 | 27.602 | 1.00 | 68.20 | O |
| ATOM | 16049 | N | GLU | C | 255 | 23.714 | 24.865 | 29.712 | 1.00 | 69.98 | N |
| ATOM | 16051 | CA | GLU | C | 255 | 24.513 | 23.729 | 29.447 | 1.00 | 69.15 | C |
| ATOM | 16053 | CB | GLU | C | 255 | 25.990 | 23.900 | 29.778 | 1.00 | 69.01 | C |
| ATOM | 16056 | CG | GLU | C | 255 | 26.765 | 24.517 | 28.627 | 1.00 | 73.89 | C |
| ATOM | 16059 | CD | GLU | C | 255 | 28.135 | 23.893 | 28.412 | 1.00 | 80.19 | C |
| ATOM | 16060 | OE1 | GLU | C | 255 | 28.259 | 22.606 | 28.391 | 1.00 | 77.88 | O |
| ATOM | 16061 | OE2 | GLU | C | 255 | 29.078 | 24.723 | 28.256 | 1.00 | 75.03 | O |
| ATOM | 16062 | C | GLU | C | 255 | 23.813 | 22.915 | 30.451 | 1.00 | 68.56 | C |
| ATOM | 16063 | O | GLU | C | 255 | 23.260 | 23.424 | 31.432 | 1.00 | 63.70 | O |
| ATOM | 16064 | N | LYS | C | 256 | 23.797 | 21.633 | 30.201 | 1.00 | 69.73 | N |
| ATOM | 16066 | CA | LYS | C | 256 | 23.140 | 20.788 | 31.117 | 1.00 | 69.57 | C |
| ATOM | 16068 | CB | LYS | C | 256 | 21.875 | 20.222 | 30.474 | 1.00 | 71.79 | C |
| ATOM | 16071 | CG | LYS | C | 256 | 20.817 | 21.373 | 30.293 | 1.00 | 73.04 | C |
| ATOM | 16074 | CD | LYS | C | 256 | 20.475 | 22.027 | 31.651 | 1.00 | 75.70 | C |
| ATOM | 16077 | CE | LYS | C | 256 | 19.065 | 22.620 | 31.712 | 1.00 | 74.59 | C |
| ATOM | 16080 | NZ | LYS | C | 256 | 18.582 | 23.111 | 30.395 | 1.00 | 76.39 | N |
| ATOM | 16084 | C | LYS | C | 256 | 24.219 | 19.828 | 31.484 | 1.00 | 67.18 | C |
| ATOM | 16085 | O | LYS | C | 256 | 25.204 | 19.623 | 30.763 | 1.00 | 58.20 | O |
| ATOM | 16086 | N | PHE | C | 257 | 24.071 | 19.275 | 32.655 | 1.00 | 68.64 | N |
| ATOM | 16088 | CA | PHE | C | 257 | 25.041 | 18.341 | 33.060 | 1.00 | 69.01 | C |
| ATOM | 16090 | CB | PHE | C | 257 | 26.005 | 19.050 | 34.009 | 1.00 | 72.10 | C |
| ATOM | 16093 | CG | PHE | C | 257 | 26.744 | 20.209 | 33.362 | 1.00 | 69.14 | C |
| ATOM | 16094 | CD1 | PHE | C | 257 | 26.419 | 21.522 | 33.680 | 1.00 | 69.09 | C |
| ATOM | 16096 | CE1 | PHE | C | 257 | 27.086 | 22.590 | 33.094 | 1.00 | 67.65 | C |
| ATOM | 16098 | CZ | PHE | C | 257 | 28.091 | 22.354 | 32.180 | 1.00 | 71.28 | C |
| ATOM | 16100 | CE2 | PHE | C | 257 | 28.431 | 21.043 | 31.849 | 1.00 | 72.57 | C |
| ATOM | 16102 | CD2 | PHE | C | 257 | 27.755 | 19.980 | 32.439 | 1.00 | 71.04 | C |
| ATOM | 16104 | C | PHE | C | 257 | 24.335 | 17.131 | 33.647 | 1.00 | 69.41 | C |
| ATOM | 16105 | O | PHE | C | 257 | 23.173 | 17.187 | 34.100 | 1.00 | 68.36 | O |
| ATOM | 16106 | N | PRO | C | 258 | 25.041 | 16.022 | 33.594 | 1.00 | 68.72 | N |
| ATOM | 16107 | CA | PRO | C | 258 | 24.559 | 14.751 | 34.139 | 1.00 | 72.08 | C |
| ATOM | 16109 | CB | PRO | C | 258 | 25.649 | 13.798 | 33.704 | 1.00 | 72.60 | C |
| ATOM | 16112 | CG | PRO | C | 258 | 26.863 | 14.705 | 33.627 | 1.00 | 72.00 | C |
| ATOM | 16115 | CD | PRO | C | 258 | 26.363 | 15.901 | 32.963 | 1.00 | 68.28 | C |
| ATOM | 16118 | C | PRO | C | 258 | 24.507 | 14.757 | 35.662 | 1.00 | 74.57 | C |
| ATOM | 16119 | O | PRO | C | 258 | 25.438 | 15.317 | 36.245 | 1.00 | 75.66 | O |
| ATOM | 16120 | N | ASP | C | 259 | 23.479 | 14.151 | 36.272 | 1.00 | 75.85 | N |
| ATOM | 16122 | CA | ASP | C | 259 | 23.355 | 14.015 | 37.747 | 1.00 | 77.50 | C |
| ATOM | 16124 | CB | ASP | C | 259 | 22.436 | 12.845 | 38.118 | 1.00 | 77.91 | C |
| ATOM | 16127 | CG | ASP | C | 259 | 20.964 | 13.134 | 37.904 | 1.00 | 80.16 | C |
| ATOM | 16128 | OD1 | ASP | C | 259 | 20.559 | 14.322 | 37.914 | 1.00 | 84.49 | O |
| ATOM | 16129 | OD2 | ASP | C | 259 | 20.138 | 12.206 | 37.727 | 1.00 | 74.07 | O |
| ATOM | 16130 | C | ASP | C | 259 | 24.649 | 13.750 | 38.543 | 1.00 | 77.00 | C |
| ATOM | 16131 | O | ASP | C | 259 | 24.727 | 14.079 | 39.732 | 1.00 | 79.56 | O |
| ATOM | 16132 | N | GLY | C | 260 | 25.648 | 13.148 | 37.914 | 1.00 | 75.01 | N |
| ATOM | 16134 | CA | GLY | C | 260 | 26.852 | 12.769 | 38.636 | 1.00 | 75.71 | C |
| ATOM | 16137 | C | GLY | C | 260 | 27.782 | 13.929 | 38.922 | 1.00 | 74.37 | C |
| ATOM | 16138 | O | GLY | C | 260 | 28.535 | 13.928 | 39.919 | 1.00 | 75.33 | O |
| ATOM | 16139 | N | PHE | C | 261 | 27.730 | 14.918 | 38.035 | 1.00 | 69.34 | N |
| ATOM | 16141 | CA | PHE | C | 261 | 28.550 | 16.095 | 38.171 | 1.00 | 62.53 | C |
| ATOM | 16143 | CB | PHE | C | 261 | 28.341 | 17.037 | 36.990 | 1.00 | 63.25 | C |
| ATOM | 16146 | CG | PHE | C | 261 | 29.079 | 18.334 | 37.098 | 1.00 | 57.93 | C |
| ATOM | 16147 | CD1 | PHE | C | 261 | 30.455 | 18.360 | 37.184 | 1.00 | 55.84 | C |
| ATOM | 16149 | CE1 | PHE | C | 261 | 31.129 | 19.557 | 37.281 | 1.00 | 55.60 | C |
| ATOM | 16151 | CZ | PHE | C | 261 | 30.424 | 20.732 | 37.293 | 1.00 | 55.30 | C |
| ATOM | 16153 | CE2 | PHE | C | 261 | 29.056 | 20.715 | 37.210 | 1.00 | 56.24 | C |
| ATOM | 16155 | CD2 | PHE | C | 261 | 28.393 | 19.522 | 37.110 | 1.00 | 55.44 | C |
| ATOM | 16157 | C | PHE | C | 261 | 28.026 | 16.693 | 39.413 | 1.00 | 58.82 | C |
| ATOM | 16158 | O | PHE | C | 261 | 28.717 | 16.758 | 40.395 | 1.00 | 61.51 | O |
| ATOM | 16159 | N | TRP | C | 262 | 26.772 | 17.095 | 39.384 | 1.00 | 58.00 | N |
| ATOM | 16161 | CA | TRP | C | 262 | 26.179 | 17.725 | 40.536 | 1.00 | 59.65 | C |
| ATOM | 16163 | CB | TRP | C | 262 | 24.722 | 18.071 | 40.271 | 1.00 | 64.16 | C |
| ATOM | 16166 | CG | TRP | C | 262 | 24.498 | 19.084 | 39.179 | 1.00 | 67.86 | C |
| ATOM | 16167 | CD1 | TRP | C | 262 | 23.762 | 18.900 | 38.046 | 1.00 | 69.53 | C |
| ATOM | 16169 | NE1 | TRP | C | 262 | 23.775 | 20.038 | 37.278 | 1.00 | 70.01 | N |
| ATOM | 16171 | CE2 | TRP | C | 262 | 24.527 | 20.992 | 37.910 | 1.00 | 68.68 | C |
| ATOM | 16172 | CD2 | TRP | C | 262 | 24.995 | 20.427 | 39.113 | 1.00 | 67.00 | C |
| ATOM | 16173 | CE3 | TRP | C | 262 | 25.793 | 21.210 | 39.941 | 1.00 | 69.48 | C |
| ATOM | 16175 | CZ3 | TRP | C | 262 | 26.091 | 22.516 | 39.547 | 1.00 | 69.43 | C |
| ATOM | 16177 | CH2 | TRP | C | 262 | 25.608 | 23.038 | 38.351 | 1.00 | 66.24 | C |
| ATOM | 16179 | CZ2 | TRP | C | 262 | 24.830 | 22.295 | 37.520 | 1.00 | 66.76 | C |
| ATOM | 16181 | C | TRP | C | 262 | 26.284 | 16.847 | 41.766 | 1.00 | 58.97 | C |
| ATOM | 16182 | O | TRP | C | 262 | 26.059 | 17.301 | 42.869 | 1.00 | 57.31 | O |
| ATOM | 16183 | N | LEU | C | 263 | 26.601 | 15.574 | 41.597 | 1.00 | 65.06 | N |
| ATOM | 16185 | CA | LEU | C | 263 | 26.806 | 14.727 | 42.767 | 1.00 | 67.45 | C |
| ATOM | 16187 | CB | LEU | C | 263 | 26.279 | 13.332 | 42.533 | 1.00 | 68.47 | C |
| ATOM | 16190 | CG | LEU | C | 263 | 24.933 | 13.109 | 43.225 | 1.00 | 72.82 | C |
| ATOM | 16192 | CD1 | LEU | C | 263 | 24.425 | 11.685 | 42.977 | 1.00 | 71.73 | C |
| ATOM | 16196 | CD2 | LEU | C | 263 | 25.028 | 13.405 | 44.740 | 1.00 | 73.38 | C |
| ATOM | 16200 | C | LEU | C | 263 | 28.268 | 14.668 | 43.160 | 1.00 | 70.29 | C |
| ATOM | 16201 | O | LEU | C | 263 | 28.647 | 13.948 | 44.076 | 1.00 | 72.79 | O |
| ATOM | 16202 | N | GLY | C | 264 | 29.083 | 15.429 | 42.449 | 1.00 | 70.41 | N |
| ATOM | 16204 | CA | GLY | C | 264 | 30.488 | 15.522 | 42.741 | 1.00 | 72.58 | C |
| ATOM | 16207 | C | GLY | C | 264 | 31.254 | 14.276 | 42.369 | 1.00 | 74.99 | C |
| ATOM | 16208 | O | GLY | C | 264 | 32.427 | 14.136 | 42.728 | 1.00 | 77.68 | O |
| ATOM | 16209 | N | GLY | C | 265 | 30.623 | 13.377 | 41.627 | 1.00 | 76.35 | N |
| ATOM | 16211 | CA | GLU | C | 265 | 31.266 | 12.097 | 41.328 | 1.00 | 75.36 | C |
| ATOM | 16213 | CB | GLU | C | 265 | 30.247 | 10.967 | 41.441 | 1.00 | 75.63 | C |
| ATOM | 16216 | CG | GLU | C | 265 | 30.068 | 10.546 | 42.883 | 1.00 | 80.22 | C |
| ATOM | 16219 | CD | GLU | C | 265 | 28.981 | 9.514 | 43.067 | 1.00 | 86.58 | C |
| ATOM | 16220 | OE1 | GLU | C | 265 | 28.455 | 9.405 | 44.208 | 1.00 | 91.85 | O |
| ATOM | 16221 | OE2 | GLU | C | 265 | 28.654 | 8.812 | 42.080 | 1.00 | 88.76 | O |
| ATOM | 16222 | C | GLU | C | 265 | 31.978 | 12.017 | 39.999 | 1.00 | 73.02 | C |
| ATOM | 16223 | O | GLU | C | 265 | 32.902 | 11.222 | 39.835 | 1.00 | 76.03 | O |
| ATOM | 16224 | N | GLN | C | 266 | 31.566 | 12.831 | 39.048 | 1.00 | 72.24 | N |
| ATOM | 16226 | CA | GLN | C | 266 | 32.162 | 12.771 | 37.730 | 1.00 | 73.00 | C |
| ATOM | 16228 | CB | GLN | C | 266 | 31.280 | 12.063 | 36.723 | 1.00 | 75.94 | C |
| ATOM | 16231 | CG | GLN | C | 266 | 29.788 | 12.214 | 36.972 | 1.00 | 81.31 | C |
| ATOM | 16234 | CD | GLN | C | 266 | 29.003 | 11.021 | 36.409 | 1.00 | 87.99 | C |
| ATOM | 16235 | OE1 | GLN | C | 266 | 28.246 | 10.351 | 37.140 | 1.00 | 89.26 | O |
| ATOM | 16236 | NE2 | GLN | C | 266 | 29.191 | 10.748 | 35.112 | 1.00 | 87.50 | N |
| ATOM | 16239 | C | GLN | C | 266 | 32.393 | 14.164 | 37.330 | 1.00 | 69.61 | C |
| ATOM | 16240 | O | GLN | C | 266 | 31.739 | 15.075 | 37.808 | 1.00 | 71.73 | O |
| ATOM | 16241 | N | LEU | C | 267 | 33.331 | 14.334 | 36.433 | 1.00 | 68.39 | N |
| ATOM | 16243 | CA | LEU | C | 267 | 33.829 | 15.649 | 36.183 | 1.00 | 65.70 | C |
| ATOM | 16245 | CB | LEU | C | 267 | 35.351 | 15.611 | 36.200 | 1.00 | 66.35 | C |
| ATOM | 16248 | CG | LEU | C | 267 | 35.956 | 14.641 | 35.211 | 1.00 | 65.47 | C |
| ATOM | 16250 | CD1 | LEU | C | 267 | 35.336 | 14.904 | 33.871 | 1.00 | 70.86 | C |
| ATOM | 16254 | CD2 | LEU | C | 267 | 37.458 | 14.823 | 35.131 | 1.00 | 67.21 | C |
| ATOM | 16258 | C | LEU | C | 267 | 33.306 | 16.140 | 34.883 | 1.00 | 66.62 | C |
| ATOM | 16259 | O | LEU | C | 267 | 32.568 | 15.458 | 34.196 | 1.00 | 68.94 | O |
| ATOM | 16260 | N | VAL | C | 268 | 33.695 | 17.344 | 34.540 | 1.00 | 68.75 | N |
| ATOM | 16262 | CA | VAL | C | 268 | 33.215 | 17.954 | 33.335 | 1.00 | 69.12 | C |
| ATOM | 16264 | CB | VAL | C | 268 | 32.183 | 19.052 | 33.644 | 1.00 | 65.32 | C |
| ATOM | 16266 | CG1 | VAL | C | 268 | 31.593 | 19.635 | 32.397 | 1.00 | 67.75 | C |
| ATOM | 16270 | CG2 | VAL | C | 268 | 31.078 | 18.451 | 34.429 | 1.00 | 67.90 | C |
| ATOM | 16274 | C | VAL | C | 268 | 34.486 | 18.491 | 32.781 | 1.00 | 70.89 | C |
| ATOM | 16275 | O | VAL | C | 268 | 35.439 | 18.735 | 33.524 | 1.00 | 77.01 | O |
| ATOM | 16276 | N | CYS | C | 269 | 34.523 | 18.651 | 31.477 | 1.00 | 70.74 | N |
| ATOM | 16278 | CA | CYS | C | 269 | 35.718 | 19.116 | 30.843 | 1.00 | 71.32 | C |
| ATOM | 16280 | CB | CYS | C | 269 | 36.480 | 17.899 | 30.275 | 1.00 | 71.55 | C |
| ATOM | 16283 | SG | CYS | C | 269 | 37.148 | 16.791 | 31.565 | 1.00 | 73.61 | S |
| ATOM | 16284 | C | CYS | C | 269 | 35.301 | 20.102 | 29.776 | 1.00 | 69.44 | C |
| ATOM | 16285 | O | CYS | C | 269 | 34.176 | 20.064 | 29.278 | 1.00 | 72.40 | O |
| ATOM | 16286 | N | TRP | C | 270 | 36.213 | 20.994 | 29.444 | 1.00 | 68.17 | N |
| ATOM | 16288 | CA | TRP | C | 270 | 35.995 | 21.964 | 28.382 | 1.00 | 68.38 | C |
| ATOM | 16290 | CB | TRP | C | 270 | 35.565 | 23.322 | 28.933 | 1.00 | 68.67 | C |
| ATOM | 16293 | CG | TRP | C | 270 | 34.140 | 23.496 | 29.291 | 1.00 | 62.51 | C |
| ATOM | 16294 | CD1 | TRP | C | 270 | 33.089 | 23.710 | 28.443 | 1.00 | 63.68 | C |
| ATOM | 16296 | NE1 | TRP | C | 270 | 31.925 | 23.859 | 29.159 | 1.00 | 67.43 | N |
| ATOM | 16298 | CE2 | TRP | C | 270 | 32.215 | 23.737 | 30.491 | 1.00 | 63.27 | C |
| ATOM | 16299 | CD2 | TRP | C | 270 | 33.602 | 23.513 | 30.605 | 1.00 | 55.28 | C |
| ATOM | 16300 | CE3 | TRP | C | 270 | 34.151 | 23.368 | 31.866 | 1.00 | 54.22 | C |
| ATOM | 16302 | CZ3 | TRP | C | 270 | 33.332 | 23.436 | 32.950 | 1.00 | 62.69 | C |
| ATOM | 16304 | CH2 | TRP | C | 270 | 31.957 | 23.653 | 32.813 | 1.00 | 66.25 | C |
| ATOM | 16306 | CZ2 | TRP | C | 270 | 31.382 | 23.807 | 31.588 | 1.00 | 62.56 | C |
| ATOM | 16308 | C | TRP | C | 270 | 37.315 | 22.194 | 27.677 | 1.00 | 69.11 | C |
| ATOM | 16309 | O | TRP | C | 270 | 38.364 | 22.221 | 28.317 | 1.00 | 70.17 | O |
| ATOM | 16310 | N | GLN | C | 271 | 37.267 | 22.369 | 26.360 | 1.00 | 71.22 | N |
| ATOM | 16312 | CA | GLN | C | 271 | 38.477 | 22.588 | 25.577 | 1.00 | 69.66 | C |
| ATOM | 16314 | CB | GLN | C | 271 | 38.160 | 22.947 | 24.102 | 1.00 | 73.03 | C |
| ATOM | 16317 | CG | GLN | C | 271 | 37.704 | 21.781 | 23.172 | 1.00 | 74.99 | C |
| ATOM | 16320 | CD | GLN | C | 271 | 36.937 | 22.285 | 21.925 | 1.00 | 83.57 | C |
| ATOM | 16321 | OE1 | GLN | C | 271 | 37.039 | 23.476 | 21.547 | 1.00 | 84.53 | O |
| ATOM | 16322 | NE2 | GLN | C | 271 | 36.168 | 21.387 | 21.294 | 1.00 | 80.42 | N |
| ATOM | 16325 | C | GLN | C | 271 | 39.156 | 23.760 | 26.217 | 1.00 | 68.22 | C |
| ATOM | 16326 | O | GLN | C | 271 | 38.520 | 24.774 | 26.484 | 1.00 | 67.07 | O |
| ATOM | 16327 | N | ALA | C | 272 | 40.440 | 23.630 | 26.477 | 1.00 | 71.24 | N |
| ATOM | 16329 | CA | ALA | C | 272 | 41.238 | 24.745 | 26.979 | 1.00 | 74.33 | C |
| ATOM | 16331 | CB | ALA | C | 272 | 42.541 | 24.817 | 26.195 | 1.00 | 74.83 | C |
| ATOM | 16335 | C | ALA | C | 272 | 40.536 | 26.126 | 26.949 | 1.00 | 74.62 | C |
| ATOM | 16336 | O | ALA | C | 272 | 39.503 | 26.332 | 26.311 | 1.00 | 71.88 | O |
| ATOM | 16337 | N | GLY | C | 273 | 41.122 | 27.065 | 27.674 | 1.00 | 76.58 | N |
| ATOM | 16339 | CA | GLY | C | 273 | 40.622 | 28.420 | 27.784 | 1.00 | 76.43 | C |
| ATOM | 16342 | C | GLY | C | 273 | 39.135 | 28.692 | 27.603 | 1.00 | 77.74 | C |
| ATOM | 16343 | O | GLY | C | 273 | 38.736 | 29.848 | 27.710 | 1.00 | 77.19 | O |
| ATOM | 16344 | N | THR | C | 274 | 38.308 | 27.683 | 27.339 | 1.00 | 76.54 | N |
| ATOM | 16346 | CA | THR | C | 274 | 36.906 | 27.955 | 27.012 | 1.00 | 76.18 | C |
| ATOM | 16348 | CB | THR | C | 274 | 36.486 | 27.048 | 25.827 | 1.00 | 76.87 | C |
| ATOM | 16350 | OG1 | THR | C | 274 | 36.826 | 25.697 | 26.109 | 1.00 | 74.47 | O |
| ATOM | 16352 | CG2 | THR | C | 274 | 37.342 | 27.319 | 24.594 | 1.00 | 80.60 | C |
| ATOM | 16356 | C | THR | C | 274 | 35.850 | 27.855 | 28.124 | 1.00 | 74.88 | C |
| ATOM | 16357 | O | THR | C | 274 | 34.676 | 28.013 | 27.834 | 1.00 | 80.88 | O |
| ATOM | 16358 | N | THR | C | 275 | 36.229 | 27.608 | 29.375 | 1.00 | 70.60 | N |
| ATOM | 16360 | CA | THR | C | 275 | 35.217 | 27.451 | 30.431 | 1.00 | 64.63 | C |
| ATOM | 16362 | CB | THR | C | 275 | 35.871 | 27.307 | 31.815 | 1.00 | 62.69 | C |
| ATOM | 16364 | OG1 | THR | C | 275 | 36.891 | 26.304 | 31.783 | 1.00 | 58.88 | O |
| ATOM | 16366 | CG2 | THR | C | 275 | 34.862 | 26.769 | 32.815 | 1.00 | 60.08 | C |
| ATOM | 16370 | C | THR | C | 275 | 34.276 | 28.641 | 30.467 | 1.00 | 59.66 | C |
| ATOM | 16371 | O | THR | C | 275 | 34.748 | 29.751 | 30.547 | 1.00 | 55.72 | O |
| ATOM | 16372 | N | PRO | C | 276 | 32.960 | 28.443 | 30.415 | 1.00 | 56.44 | N |
| ATOM | 16373 | CA | PRO | C | 276 | 32.060 | 29.588 | 30.442 | 1.00 | 55.38 | C |
| ATOM | 16375 | CB | PRO | C | 276 | 30.771 | 28.992 | 29.922 | 1.00 | 57.23 | C |
| ATOM | 16378 | CG | PRO | C | 276 | 30.763 | 27.713 | 30.617 | 1.00 | 56.39 | C |
| ATOM | 16381 | CD | PRO | C | 276 | 32.182 | 27.190 | 30.320 | 1.00 | 57.95 | C |
| ATOM | 16384 | C | PRO | C | 276 | 31.872 | 30.073 | 31.857 | 1.00 | 52.50 | C |
| ATOM | 16385 | O | PRO | C | 276 | 30.764 | 30.068 | 32.414 | 1.00 | 56.88 | O |
| ATOM | 16386 | N | TRP | C | 277 | 32.951 | 30.527 | 32.459 | 1.00 | 49.70 | N |
| ATOM | 16388 | CA | TRP | C | 277 | 32.835 | 30.898 | 33.842 | 1.00 | 48.70 | C |
| ATOM | 16390 | CB | TRP | C | 277 | 34.047 | 31.691 | 34.288 | 1.00 | 48.72 | C |
| ATOM | 16393 | CG | TRP | C | 277 | 35.377 | 30.992 | 34.300 | 1.00 | 42.99 | C |
| ATOM | 16394 | CD1 | TRP | C | 277 | 36.452 | 31.282 | 33.497 | 1.00 | 44.54 | C |
| ATOM | 16396 | NE1 | TRP | C | 277 | 37.517 | 30.463 | 33.795 | 1.00 | 40.79 | N |
| ATOM | 16398 | CE2 | TRP | C | 277 | 37.153 | 29.623 | 34.816 | 1.00 | 41.09 | C |
| ATOM | 16399 | CD2 | TRP | C | 277 | 35.803 | 29.931 | 35.159 | 1.00 | 42.82 | C |
| ATOM | 16400 | CE3 | TRP | C | 277 | 35.193 | 29.196 | 36.190 | 1.00 | 43.92 | C |
| ATOM | 16402 | CZ3 | TRP | C | 277 | 35.938 | 28.193 | 36.833 | 1.00 | 34.69 | C |
| ATOM | 16404 | CH2 | TRP | C | 277 | 37.274 | 27.915 | 36.441 | 1.00 | 31.59 | C |
| ATOM | 16406 | CZ2 | TRP | C | 277 | 37.888 | 28.619 | 35.454 | 1.00 | 28.84 | C |
| ATOM | 16408 | C | TRP | C | 277 | 31.559 | 31.719 | 34.043 | 1.00 | 50.14 | C |
| ATOM | 16409 | O | TRP | C | 277 | 30.821 | 31.528 | 35.002 | 1.00 | 53.55 | O |
| ATOM | 16410 | N | ASN | C | 278 | 31.287 | 32.633 | 33.120 | 1.00 | 53.60 | N |
| ATOM | 16412 | CA | ASN | C | 278 | 30.173 | 33.561 | 33.273 | 1.00 | 50.74 | C |
| ATOM | 16414 | CB | ASN | C | 278 | 30.280 | 34.662 | 32.191 | 1.00 | 54.60 | C |
| ATOM | 16417 | CG | ASN | C | 278 | 29.472 | 34.370 | 30.928 | 1.00 | 57.61 | C |
| ATOM | 16418 | OD1 | ASN | C | 278 | 28.305 | 34.753 | 30.826 | 1.00 | 62.78 | O |
| ATOM | 16419 | ND2 | ASN | C | 278 | 30.101 | 33.723 | 29.951 | 1.00 | 50.67 | N |
| ATOM | 16422 | C | ASN | C | 278 | 28.767 | 32.886 | 33.392 | 1.00 | 52.28 | C |
| ATOM | 16423 | O | ASN | C | 278 | 27.865 | 33.415 | 34.082 | 1.00 | 46.13 | O |
| ATOM | 16424 | N | ILE | C | 279 | 28.582 | 31.714 | 32.777 | 1.00 | 52.83 | N |
| ATOM | 16426 | CA | ILE | C | 279 | 27.287 | 31.048 | 32.896 | 1.00 | 56.47 | C |
| ATOM | 16428 | CB | ILE | C | 279 | 27.104 | 29.838 | 31.946 | 1.00 | 57.69 | C |
| ATOM | 16430 | CG1 | ILE | C | 279 | 27.859 | 28.617 | 32.471 | 1.00 | 57.27 | C |
| ATOM | 16433 | CD1 | ILE | C | 279 | 27.534 | 27.307 | 31.703 | 1.00 | 54.35 | C |
| ATOM | 16437 | CG2 | ILE | C | 279 | 27.493 | 30.172 | 30.507 | 1.00 | 58.04 | C |
| ATOM | 16441 | C | ILE | C | 279 | 27.047 | 30.544 | 34.300 | 1.00 | 57.88 | C |
| ATOM | 16442 | O | ILE | C | 279 | 25.936 | 30.141 | 34.610 | 1.00 | 59.87 | O |
| ATOM | 16443 | N | PHE | C | 280 | 28.078 | 30.544 | 35.144 | 1.00 | 56.99 | N |
| ATOM | 16445 | CA | PHE | C | 280 | 27.934 | 30.040 | 36.500 | 1.00 | 51.07 | C |
| ATOM | 16447 | CB | PHE | C | 280 | 29.142 | 29.215 | 36.869 | 1.00 | 54.92 | C |
| ATOM | 16450 | CG | PHE | C | 280 | 29.180 | 27.861 | 36.227 | 1.00 | 51.56 | C |
| ATOM | 16451 | CD1 | PHE | C | 280 | 28.625 | 26.777 | 36.852 | 1.00 | 48.09 | C |
| ATOM | 16453 | CE1 | PHE | C | 280 | 28.675 | 25.583 | 36.298 | 1.00 | 46.71 | C |
| ATOM | 16455 | CZ | PHE | C | 280 | 29.262 | 25.412 | 35.110 | 1.00 | 53.19 | C |
| ATOM | 16457 | CE2 | PHE | C | 280 | 29.822 | 26.462 | 34.471 | 1.00 | 52.26 | C |
| ATOM | 16459 | CD2 | PHE | C | 280 | 29.781 | 27.683 | 35.031 | 1.00 | 48.92 | C |
| ATOM | 16461 | C | PHE | C | 280 | 27.888 | 31.203 | 37.432 | 1.00 | 48.27 | C |
| ATOM | 16462 | O | PHE | C | 280 | 28.646 | 32.139 | 37.277 | 1.00 | 47.19 | O |
| ATOM | 16463 | N | PRO | C | 281 | 27.031 | 31.141 | 38.427 | 1.00 | 45.62 | N |
| ATOM | 16464 | CA | PRO | C | 281 | 26.801 | 32.279 | 39.285 | 1.00 | 47.19 | C |
| ATOM | 16466 | CB | PRO | C | 281 | 25.373 | 32.029 | 39.741 | 1.00 | 48.28 | C |
| ATOM | 16469 | CG | PRO | C | 281 | 25.269 | 30.542 | 39.864 | 1.00 | 44.59 | C |
| ATOM | 16472 | CD | PRO | C | 281 | 26.237 | 29.991 | 38.869 | 1.00 | 48.35 | C |
| ATOM | 16475 | C | PRO | C | 281 | 27.761 | 32.335 | 40.463 | 1.00 | 48.66 | C |
| ATOM | 16476 | O | PRO | C | 281 | 28.525 | 31.410 | 40.674 | 1.00 | 56.99 | O |
| ATOM | 16477 | N | VAL | C | 282 | 27.712 | 33.423 | 41.214 | 1.00 | 46.97 | N |
| ATOM | 16479 | CA | VAL | C | 282 | 28.597 | 33.628 | 42.345 | 1.00 | 44.44 | C |
| ATOM | 16481 | CB | VAL | C | 282 | 29.071 | 35.069 | 42.430 | 1.00 | 44.57 | C |
| ATOM | 16483 | CG1 | VAL | C | 282 | 29.756 | 35.469 | 41.118 | 1.00 | 45.56 | C |
| ATOM | 16487 | CG2 | VAL | C | 282 | 27.890 | 36.003 | 42.759 | 1.00 | 34.25 | C |
| ATOM | 16491 | C | VAL | C | 282 | 27.739 | 33.349 | 43.545 | 1.00 | 45.50 | C |
| ATOM | 16492 | O | VAL | C | 282 | 26.506 | 33.444 | 43.451 | 1.00 | 42.57 | O |
| ATOM | 16493 | N | ILE | C | 283 | 28.375 | 33.027 | 44.664 | 1.00 | 38.05 | N |
| ATOM | 16495 | CA | ILE | C | 283 | 27.636 | 32.624 | 45.821 | 1.00 | 39.60 | C |
| ATOM | 16497 | CB | ILE | C | 283 | 27.997 | 31.210 | 46.205 | 1.00 | 42.77 | C |
| ATOM | 16499 | CG1 | ILE | C | 283 | 27.296 | 30.218 | 45.282 | 1.00 | 45.31 | C |
| ATOM | 16502 | CD1 | ILE | C | 283 | 28.010 | 28.859 | 45.202 | 1.00 | 51.32 | C |
| ATOM | 16506 | CG2 | ILE | C | 283 | 27.494 | 30.935 | 47.573 | 1.00 | 48.65 | C |
| ATOM | 16510 | C | ILE | C | 283 | 27.951 | 33.519 | 46.938 | 1.00 | 36.72 | C |
| ATOM | 16511 | O | ILE | C | 283 | 29.060 | 33.947 | 47.071 | 1.00 | 39.41 | O |
| ATOM | 16512 | N | SER | C | 284 | 26.964 | 33.814 | 47.765 | 1.00 | 43.61 | N |
| ATOM | 16514 | CA | SER | C | 284 | 27.196 | 34.724 | 48.883 | 1.00 | 42.34 | C |
| ATOM | 16516 | CB | SER | C | 284 | 26.479 | 36.048 | 48.680 | 1.00 | 40.69 | C |
| ATOM | 16519 | OG | SER | C | 284 | 26.967 | 36.700 | 47.532 | 1.00 | 40.19 | O |
| ATOM | 16521 | C | SER | C | 284 | 26.765 | 34.148 | 50.197 | 1.00 | 42.31 | C |
| ATOM | 16522 | O | SER | C | 284 | 25.721 | 33.515 | 50.367 | 1.00 | 42.00 | O |
| ATOM | 16523 | N | LEU | C | 285 | 27.605 | 34.361 | 51.159 | 1.00 | 40.93 | N |
| ATOM | 16525 | CA | LEU | C | 285 | 27.239 | 33.946 | 52.450 | 1.00 | 40.64 | C |
| ATOM | 16527 | CB | LEU | C | 285 | 28.263 | 32.934 | 52.951 | 1.00 | 43.13 | C |
| ATOM | 16530 | CG | LEU | C | 285 | 28.493 | 31.677 | 52.107 | 1.00 | 43.50 | C |
| ATOM | 16532 | CD1 | LEU | C | 285 | 29.160 | 30.630 | 52.986 | 1.00 | 45.83 | C |
| ATOM | 16536 | CD2 | LEU | C | 285 | 27.224 | 31.096 | 51.550 | 1.00 | 47.33 | C |
| ATOM | 16540 | C | LEU | C | 285 | 27.238 | 35.237 | 53.241 | 1.00 | 38.54 | C |
| ATOM | 16541 | O | LEU | C | 285 | 28.192 | 36.012 | 53.142 | 1.00 | 38.71 | O |
| ATOM | 16542 | N | TYR | C | 286 | 26.182 | 35.508 | 54.004 | 1.00 | 41.40 | N |
| ATOM | 16544 | CA | TYR | C | 286 | 26.201 | 36.697 | 54.882 | 1.00 | 43.34 | C |
| ATOM | 16546 | CB | TYR | C | 286 | 24.868 | 37.456 | 54.919 | 1.00 | 43.01 | C |
| ATOM | 16549 | CG | TYR | C | 286 | 24.458 | 38.178 | 53.648 | 1.00 | 43.98 | C |
| ATOM | 16550 | CD1 | TYR | C | 286 | 24.169 | 37.480 | 52.485 | 1.00 | 41.24 | C |
| ATOM | 16552 | CE1 | TYR | C | 286 | 23.796 | 38.118 | 51.350 | 1.00 | 37.43 | C |
| ATOM | 16554 | CZ | TYR | C | 286 | 23.683 | 39.479 | 51.318 | 1.00 | 38.55 | C |
| ATOM | 16555 | OH | TYR | C | 286 | 23.279 | 40.098 | 50.129 | 1.00 | 31.80 | O |
| ATOM | 16557 | CE2 | TYR | C | 286 | 23.950 | 40.197 | 52.451 | 1.00 | 36.26 | C |
| ATOM | 16559 | CD2 | TYR | C | 286 | 24.331 | 39.541 | 53.615 | 1.00 | 41.46 | C |
| ATOM | 16561 | C | TYR | C | 286 | 26.525 | 36.179 | 56.278 | 1.00 | 40.55 | C |
| ATOM | 16562 | O | TYR | C | 286 | 25.810 | 35.340 | 56.792 | 1.00 | 35.99 | O |
| ATOM | 16563 | N | LEU | C | 287 | 27.609 | 36.668 | 56.876 | 1.00 | 43.12 | N |
| ATOM | 16565 | CA | LEU | C | 287 | 27.992 | 36.203 | 58.201 | 1.00 | 44.11 | C |
| ATOM | 16567 | CB | LEU | C | 287 | 29.451 | 35.807 | 58.319 | 1.00 | 44.04 | C |
| ATOM | 16570 | CG | LEU | C | 287 | 30.022 | 34.821 | 57.316 | 1.00 | 45.36 | C |
| ATOM | 16572 | CD1 | LEU | C | 287 | 31.494 | 35.117 | 57.164 | 1.00 | 48.19 | C |
| ATOM | 16576 | CD2 | LEU | C | 287 | 29.854 | 33.410 | 57.786 | 1.00 | 49.68 | C |
| ATOM | 16580 | C | LEU | C | 287 | 27.750 | 37.322 | 59.116 | 1.00 | 41.05 | C |
| ATOM | 16581 | O | LEU | C | 287 | 27.724 | 38.435 | 58.705 | 1.00 | 45.70 | O |
| ATOM | 16582 | N | MET | C | 288 | 27.578 | 36.981 | 60.370 | 1.00 | 48.49 | N |
| ATOM | 16584 | CA | MET | C | 288 | 27.201 | 37.884 | 61.414 | 1.00 | 50.98 | C |
| ATOM | 16586 | CB | MET | C | 288 | 26.743 | 37.033 | 62.614 | 1.00 | 53.16 | C |
| ATOM | 16589 | CG | MET | C | 288 | 26.255 | 37.824 | 63.814 | 1.00 | 59.82 | C |
| ATOM | 16592 | SD | MET | C | 288 | 25.588 | 36.852 | 65.181 | 1.00 | 66.84 | S |
| ATOM | 16593 | CE | MET | C | 288 | 24.400 | 35.852 | 64.340 | 1.00 | 65.00 | C |
| ATOM | 16597 | C | MET | C | 288 | 28.448 | 38.645 | 61.724 | 1.00 | 52.60 | C |
| ATOM | 16598 | O | MET | C | 288 | 29.541 | 38.109 | 61.660 | 1.00 | 49.84 | O |
| ATOM | 16599 | N | GLY | C | 289 | 28.298 | 39.906 | 62.056 | 1.00 | 53.80 | N |
| ATOM | 16601 | CA | GLY | C | 289 | 29.458 | 40.726 | 62.323 | 1.00 | 53.77 | C |
| ATOM | 16604 | C | GLY | C | 289 | 29.552 | 41.029 | 63.794 | 1.00 | 52.86 | C |
| ATOM | 16605 | O | GLY | C | 289 | 28.759 | 40.561 | 64.560 | 1.00 | 55.64 | O |
| ATOM | 16606 | N | GLU | C | 290 | 30.527 | 41.828 | 64.185 | 1.00 | 59.63 | N |
| ATOM | 16608 | CA | GLU | C | 290 | 30.713 | 42.194 | 65.586 | 1.00 | 62.62 | C |
| ATOM | 16610 | CB | GLU | C | 290 | 32.131 | 42.676 | 65.770 | 1.00 | 61.72 | C |
| ATOM | 16613 | CG | GLU | C | 290 | 33.118 | 41.620 | 65.403 | 1.00 | 56.49 | C |
| ATOM | 16616 | CD | GLU | C | 290 | 34.316 | 41.728 | 66.263 | 1.00 | 62.75 | C |
| ATOM | 16617 | OE1 | GLU | C | 290 | 35.173 | 42.598 | 65.952 | 1.00 | 66.45 | O |
| ATOM | 16618 | OE2 | GLU | C | 290 | 34.368 | 40.951 | 67.245 | 1.00 | 58.63 | O |
| ATOM | 16619 | C | GLU | C | 290 | 29.799 | 43.253 | 66.185 | 1.00 | 66.84 | C |
| ATOM | 16620 | O | GLU | C | 290 | 29.553 | 43.241 | 67.411 | 1.00 | 65.72 | O |
| ATOM | 16621 | N | VAL | C | 291 | 29.309 | 44.171 | 65.356 | 1.00 | 71.13 | N |
| ATOM | 16623 | CA | VAL | C | 291 | 28.433 | 45.221 | 65.869 | 1.00 | 74.55 | C |
| ATOM | 16625 | CB | VAL | C | 291 | 28.539 | 46.525 | 65.086 | 1.00 | 76.24 | C |
| ATOM | 16627 | CG1 | VAL | C | 291 | 27.577 | 47.563 | 65.658 | 1.00 | 76.47 | C |
| ATOM | 16631 | CG2 | VAL | C | 291 | 29.953 | 47.059 | 65.150 | 1.00 | 81.20 | C |
| ATOM | 16635 | C | VAL | C | 291 | 27.003 | 44.778 | 65.840 | 1.00 | 75.19 | C |
| ATOM | 16636 | O | VAL | C | 291 | 26.553 | 44.138 | 64.875 | 1.00 | 76.87 | O |
| ATOM | 16637 | N | THR | C | 292 | 26.299 | 45.129 | 66.912 | 1.00 | 76.63 | N |
| ATOM | 16639 | CA | THR | C | 292 | 24.884 | 44.803 | 67.090 | 1.00 | 78.73 | C |
| ATOM | 16641 | CB | THR | C | 292 | 24.380 | 45.595 | 68.298 | 1.00 | 79.80 | C |
| ATOM | 16643 | OG1 | THR | C | 292 | 25.508 | 46.083 | 69.035 | 1.00 | 83.48 | O |
| ATOM | 16645 | CG2 | THR | C | 292 | 23.660 | 44.683 | 69.301 | 1.00 | 82.75 | C |
| ATOM | 16649 | C | THR | C | 292 | 24.039 | 45.118 | 65.842 | 1.00 | 78.20 | C |
| ATOM | 16650 | O | THR | C | 292 | 24.152 | 46.206 | 65.281 | 1.00 | 76.52 | O |
| ATOM | 16651 | N | GLN | C | 293 | 23.212 | 44.153 | 65.414 | 1.00 | 77.10 | N |
| ATOM | 16653 | CA | GLN | C | 293 | 22.352 | 44.280 | 64.223 | 1.00 | 73.84 | C |
| ATOM | 16655 | CB | GLN | C | 293 | 21.520 | 45.562 | 64.352 | 1.00 | 79.00 | C |
| ATOM | 16658 | CG | GLN | C | 293 | 20.073 | 45.347 | 64.881 | 1.00 | 85.11 | C |
| ATOM | 16661 | CD | GLN | C | 293 | 19.749 | 46.139 | 66.165 | 1.00 | 87.31 | C |
| ATOM | 16662 | OE1 | GLN | C | 293 | 18.960 | 45.672 | 67.007 | 1.00 | 85.12 | O |
| ATOM | 16663 | NE2 | GLN | C | 293 | 20.347 | 47.329 | 66.308 | 1.00 | 86.21 | N |
| ATOM | 16666 | C | GLN | C | 293 | 23.045 | 44.268 | 62.842 | 1.00 | 65.85 | C |
| ATOM | 16667 | O | GLN | C | 293 | 22.370 | 44.359 | 61.822 | 1.00 | 63.96 | O |
| ATOM | 16668 | N | GLN | C | 294 | 24.366 | 44.120 | 62.803 | 1.00 | 66.61 | N |
| ATOM | 16670 | CA | GLN | C | 294 | 25.141 | 44.364 | 61.580 | 1.00 | 61.35 | C |
| ATOM | 16672 | CB | GLN | C | 294 | 26.324 | 45.288 | 61.936 | 1.00 | 60.41 | C |
| ATOM | 16675 | CG | GLN | C | 294 | 26.837 | 46.202 | 60.839 | 1.00 | 62.21 | C |
| ATOM | 16678 | CD | GLN | C | 294 | 27.669 | 47.413 | 61.365 | 1.00 | 65.59 | C |
| ATOM | 16679 | OE1 | GLN | C | 294 | 28.765 | 47.713 | 60.847 | 1.00 | 59.99 | O |
| ATOM | 16680 | NE2 | GLN | C | 294 | 27.138 | 48.107 | 62.373 | 1.00 | 65.49 | N |
| ATOM | 16683 | C | GLN | C | 294 | 25.627 | 43.036 | 60.985 | 1.00 | 56.94 | C |
| ATOM | 16684 | O | GLN | C | 294 | 25.912 | 42.085 | 61.710 | 1.00 | 58.82 | O |
| ATOM | 16685 | N | SER | C | 295 | 25.718 | 42.958 | 59.668 | 1.00 | 46.60 | N |
| ATOM | 16687 | CA | SER | C | 295 | 26.213 | 41.760 | 59.056 | 1.00 | 42.55 | C |
| ATOM | 16689 | CB | SER | C | 295 | 25.079 | 40.909 | 58.628 | 1.00 | 40.84 | C |
| ATOM | 16692 | OG | SER | C | 295 | 24.697 | 41.439 | 57.411 | 1.00 | 43.82 | O |
| ATOM | 16694 | C | SER | C | 295 | 26.965 | 42.103 | 57.806 | 1.00 | 44.01 | C |
| ATOM | 16695 | O | SER | C | 295 | 26.877 | 43.217 | 57.316 | 1.00 | 41.78 | O |
| ATOM | 16696 | N | PHE | C | 296 | 27.688 | 41.133 | 57.260 | 1.00 | 40.42 | N |
| ATOM | 16698 | CA | PHE | C | 296 | 28.455 | 41.395 | 56.077 | 1.00 | 40.53 | C |
| ATOM | 16700 | CB | PHE | C | 296 | 29.870 | 41.876 | 56.413 | 1.00 | 42.71 | C |
| ATOM | 16703 | CG | PHE | C | 296 | 30.782 | 40.825 | 56.974 | 1.00 | 40.05 | C |
| ATOM | 16704 | CD1 | PHE | C | 296 | 31.588 | 40.081 | 56.147 | 1.00 | 43.51 | C |
| ATOM | 16706 | CE1 | PHE | C | 296 | 32.406 | 39.148 | 56.647 | 1.00 | 36.06 | C |
| ATOM | 16708 | CZ | PHE | C | 296 | 32.443 | 38.940 | 58.011 | 1.00 | 42.47 | C |
| ATOM | 16710 | CE2 | PHE | C | 296 | 31.674 | 39.657 | 58.822 | 1.00 | 37.71 | C |
| ATOM | 16712 | CD2 | PHE | C | 296 | 30.848 | 40.602 | 58.315 | 1.00 | 37.78 | C |
| ATOM | 16714 | C | PHE | C | 296 | 28.455 | 40.127 | 55.332 | 1.00 | 42.08 | C |
| ATOM | 16715 | O | PHE | C | 296 | 28.030 | 39.122 | 55.906 | 1.00 | 46.21 | O |
| ATOM | 16716 | N | ARG | C | 297 | 28.908 | 40.167 | 54.070 | 1.00 | 40.78 | N |
| ATOM | 16718 | CA | ARG | C | 297 | 28.958 | 38.974 | 53.211 | 1.00 | 44.56 | C |
| ATOM | 16720 | CB | ARG | C | 297 | 27.894 | 39.031 | 52.123 | 1.00 | 44.33 | C |
| ATOM | 16723 | CG | ARG | C | 297 | 28.197 | 40.056 | 51.064 | 1.00 | 50.40 | C |
| ATOM | 16726 | CD | ARG | C | 297 | 27.009 | 40.427 | 50.181 | 1.00 | 54.77 | C |
| ATOM | 16729 | NE | ARG | C | 297 | 27.370 | 41.445 | 49.195 | 1.00 | 56.04 | N |
| ATOM | 16731 | CZ | ARG | C | 297 | 26.505 | 42.187 | 48.535 | 1.00 | 50.69 | C |
| ATOM | 16732 | NH1 | ARG | C | 297 | 25.204 | 42.040 | 48.732 | 1.00 | 51.47 | N |
| ATOM | 16735 | NH2 | ARG | C | 297 | 26.946 | 43.079 | 47.677 | 1.00 | 46.53 | N |
| ATOM | 16738 | C | ARG | C | 297 | 30.273 | 38.740 | 52.498 | 1.00 | 42.25 | C |
| ATOM | 16739 | O | ARG | C | 297 | 31.035 | 39.648 | 52.215 | 1.00 | 47.66 | O |
| ATOM | 16740 | N | ILE | C | 298 | 30.518 | 37.488 | 52.194 | 1.00 | 41.06 | N |
| ATOM | 16742 | CA | ILE | C | 298 | 31.679 | 37.115 | 51.439 | 1.00 | 41.71 | C |
| ATOM | 16744 | CB | ILE | C | 298 | 32.517 | 36.104 | 52.264 | 1.00 | 38.04 | C |
| ATOM | 16746 | CG1 | ILE | C | 298 | 31.858 | 34.741 | 52.331 | 1.00 | 39.06 | C |
| ATOM | 16749 | CD1 | ILE | C | 298 | 32.459 | 33.774 | 53.357 | 1.00 | 42.84 | C |
| ATOM | 16753 | CG2 | ILE | C | 298 | 32.654 | 36.628 | 53.672 | 1.00 | 37.72 | C |
| ATOM | 16757 | C | ILE | C | 298 | 31.051 | 36.559 | 50.189 | 1.00 | 36.43 | C |
| ATOM | 16758 | O | ILE | C | 298 | 29.989 | 35.993 | 50.268 | 1.00 | 39.29 | O |
| ATOM | 16759 | N | THR | C | 299 | 31.675 | 36.727 | 49.041 | 1.00 | 41.37 | N |
| ATOM | 16761 | CA | THR | C | 299 | 31.098 | 36.225 | 47.769 | 1.00 | 42.98 | C |
| ATOM | 16763 | CB | THR | C | 299 | 30.626 | 37.411 | 46.893 | 1.00 | 44.37 | C |
| ATOM | 16765 | OG1 | THR | C | 299 | 30.064 | 38.411 | 47.749 | 1.00 | 48.01 | O |
| ATOM | 16767 | CG2 | THR | C | 299 | 29.455 | 37.062 | 45.991 | 1.00 | 45.15 | C |
| ATOM | 16771 | C | THR | C | 299 | 32.163 | 35.478 | 47.050 | 1.00 | 38.51 | C |
| ATOM | 16772 | O | THR | C | 299 | 33.282 | 35.947 | 46.931 | 1.00 | 38.31 | O |
| ATOM | 16773 | N | ILE | C | 300 | 31.834 | 34.309 | 46.557 | 1.00 | 40.54 | N |
| ATOM | 16775 | CA | ILE | C | 300 | 32.849 | 33.494 | 45.920 | 1.00 | 40.34 | C |
| ATOM | 16777 | CB | ILE | C | 300 | 33.019 | 32.177 | 46.685 | 1.00 | 40.87 | C |
| ATOM | 16779 | CG1 | ILE | C | 300 | 32.051 | 31.162 | 46.204 | 1.00 | 34.13 | C |
| ATOM | 16782 | CD1 | ILE | C | 300 | 32.254 | 29.851 | 46.922 | 1.00 | 36.30 | C |
| ATOM | 16786 | CG2 | ILE | C | 300 | 32.721 | 32.317 | 48.186 | 1.00 | 44.16 | C |
| ATOM | 16790 | C | ILE | C | 300 | 32.440 | 33.216 | 44.505 | 1.00 | 40.76 | C |
| ATOM | 16791 | O | ILE | C | 300 | 31.254 | 33.277 | 44.220 | 1.00 | 39.41 | O |
| ATOM | 16792 | N | LEU | C | 301 | 33.426 | 32.905 | 43.645 | 1.00 | 44.04 | N |
| ATOM | 16794 | CA | LEU | C | 301 | 33.215 | 32.633 | 42.210 | 1.00 | 45.48 | C |
| ATOM | 16796 | CB | LEU | C | 301 | 34.199 | 33.429 | 41.340 | 1.00 | 47.46 | C |
| ATOM | 16799 | CG | LEU | C | 301 | 34.544 | 34.876 | 41.679 | 1.00 | 50.30 | C |
| ATOM | 16801 | CD1 | LEU | C | 301 | 35.649 | 35.402 | 40.734 | 1.00 | 51.91 | C |
| ATOM | 16805 | CD2 | LEU | C | 301 | 33.312 | 35.727 | 41.580 | 1.00 | 48.11 | C |
| ATOM | 16809 | C | LEU | C | 301 | 33.338 | 31.183 | 41.734 | 1.00 | 46.00 | C |
| ATOM | 16810 | O | LEU | C | 301 | 33.844 | 30.283 | 42.386 | 1.00 | 40.23 | O |
| ATOM | 16811 | N | PRO | C | 302 | 32.905 | 30.973 | 40.516 | 1.00 | 47.02 | N |
| ATOM | 16812 | CA | PRO | C | 302 | 32.898 | 29.626 | 39.965 | 1.00 | 45.68 | C |
| ATOM | 16814 | CB | PRO | C | 302 | 32.276 | 29.806 | 38.584 | 1.00 | 43.48 | C |
| ATOM | 16817 | CG | PRO | C | 302 | 31.796 | 31.181 | 38.541 | 1.00 | 45.27 | C |
| ATOM | 16820 | CD | PRO | C | 302 | 32.456 | 31.994 | 39.567 | 1.00 | 42.09 | C |
| ATOM | 16823 | C | PRO | C | 302 | 34.331 | 29.104 | 39.891 | 1.00 | 48.54 | C |
| ATOM | 16824 | O | PRO | C | 302 | 34.534 | 27.886 | 39.845 | 1.00 | 49.42 | O |
| ATOM | 16825 | N | GLN | C | 303 | 35.309 | 30.014 | 39.873 | 1.00 | 47.34 | N |
| ATOM | 16827 | CA | GLN | C | 303 | 36.708 | 29.610 | 39.973 | 1.00 | 47.58 | C |
| ATOM | 16829 | CB | GLN | C | 303 | 37.637 | 30.790 | 39.736 | 1.00 | 47.37 | C |
| ATOM | 16832 | CG | GLN | C | 303 | 37.910 | 31.131 | 38.293 | 1.00 | 45.62 | C |
| ATOM | 16835 | CD | GLN | C | 303 | 36.936 | 32.113 | 37.717 | 1.00 | 45.06 | C |
| ATOM | 16836 | OE1 | GLN | C | 303 | 35.838 | 32.299 | 38.244 | 1.00 | 37.43 | O |
| ATOM | 16837 | NE2 | GLN | C | 303 | 37.334 | 32.747 | 36.617 | 1.00 | 50.52 | N |
| ATOM | 16840 | C | GLN | C | 303 | 37.068 | 28.996 | 41.346 | 1.00 | 49.02 | C |
| ATOM | 16841 | O | GLN | C | 303 | 38.159 | 28.473 | 41.514 | 1.00 | 51.60 | O |
| ATOM | 16842 | N | GLN | C | 304 | 36.169 | 29.052 | 42.316 | 1.00 | 49.47 | N |
| ATOM | 16844 | CA | GLN | C | 304 | 36.438 | 28.475 | 43.616 | 1.00 | 50.38 | C |
| ATOM | 16846 | CB | GLN | C | 304 | 36.269 | 29.511 | 44.737 | 1.00 | 52.45 | C |
| ATOM | 16849 | CG | GLN | C | 304 | 37.510 | 30.399 | 44.949 | 1.00 | 50.66 | C |
| ATOM | 16852 | CD | GLN | C | 304 | 37.438 | 31.671 | 44.151 | 1.00 | 51.45 | C |
| ATOM | 16853 | OE1 | GLN | C | 304 | 38.235 | 31.881 | 43.210 | 1.00 | 51.29 | O |
| ATOM | 16854 | NE2 | GLN | C | 304 | 36.484 | 32.532 | 44.509 | 1.00 | 40.58 | N |
| ATOM | 16857 | C | GLN | C | 304 | 35.606 | 27.249 | 43.939 | 1.00 | 51.95 | C |
| ATOM | 16858 | O | GLN | C | 304 | 36.120 | 26.322 | 44.554 | 1.00 | 55.44 | O |
| ATOM | 16859 | N | TYR | C | 305 | 34.328 | 27.212 | 43.574 | 1.00 | 56.29 | N |
| ATOM | 16861 | CA | TYR | C | 305 | 33.569 | 25.956 | 43.788 | 1.00 | 52.77 | C |
| ATOM | 16863 | CB | TYR | C | 305 | 32.064 | 26.151 | 44.100 | 1.00 | 50.80 | C |
| ATOM | 16866 | CG | TYR | C | 305 | 31.206 | 26.929 | 43.131 | 1.00 | 44.82 | C |
| ATOM | 16867 | CD1 | TYR | C | 305 | 30.417 | 26.271 | 42.212 | 1.00 | 44.88 | C |
| ATOM | 16869 | CE1 | TYR | C | 305 | 29.642 | 26.928 | 41.352 | 1.00 | 35.09 | C |
| ATOM | 16871 | CZ | TYR | C | 305 | 29.594 | 28.271 | 41.368 | 1.00 | 36.83 | C |
| ATOM | 16872 | OH | TYR | C | 305 | 28.748 | 28.910 | 40.445 | 1.00 | 41.33 | O |
| ATOM | 16874 | CE2 | TYR | C | 305 | 30.352 | 28.964 | 42.269 | 1.00 | 37.21 | C |
| ATOM | 16876 | CD2 | TYR | C | 305 | 31.149 | 28.290 | 43.155 | 1.00 | 38.78 | C |
| ATOM | 16878 | C | TYR | C | 305 | 33.788 | 24.886 | 42.730 | 1.00 | 51.03 | C |
| ATOM | 16879 | O | TYR | C | 305 | 33.369 | 23.774 | 42.952 | 1.00 | 53.32 | O |
| ATOM | 16880 | N | LEU | C | 306 | 34.465 | 25.192 | 41.618 | 1.00 | 52.84 | N |
| ATOM | 16882 | CA | LEU | C | 306 | 34.720 | 24.182 | 40.568 | 1.00 | 52.04 | C |
| ATOM | 16884 | CB | LEU | C | 306 | 34.395 | 24.713 | 39.181 | 1.00 | 54.77 | C |
| ATOM | 16887 | CG | LEU | C | 306 | 32.909 | 24.805 | 38.761 | 1.00 | 51.54 | C |
| ATOM | 16889 | CD1 | LEU | C | 306 | 32.752 | 25.575 | 37.502 | 1.00 | 44.63 | C |
| ATOM | 16893 | CD2 | LEU | C | 306 | 32.350 | 23.442 | 38.555 | 1.00 | 52.67 | C |
| ATOM | 16897 | C | LEU | C | 306 | 36.187 | 23.850 | 40.620 | 1.00 | 56.99 | C |
| ATOM | 16898 | O | LEU | C | 306 | 37.008 | 24.666 | 40.199 | 1.00 | 57.98 | O |
| ATOM | 16899 | N | ARG | C | 307 | 36.518 | 22.648 | 41.110 | 1.00 | 54.03 | N |
| ATOM | 16901 | CA | ARG | C | 307 | 37.888 | 22.323 | 41.457 | 1.00 | 54.93 | C |
| ATOM | 16903 | CB | ARG | C | 307 | 37.877 | 21.401 | 42.707 | 1.00 | 50.67 | C |
| ATOM | 16906 | CG | ARG | C | 307 | 39.207 | 20.794 | 43.096 | 1.00 | 51.43 | C |
| ATOM | 16909 | CD | ARG | C | 307 | 39.237 | 20.023 | 44.478 | 1.00 | 52.58 | C |
| ATOM | 16912 | NE | ARG | C | 307 | 38.527 | 18.734 | 44.515 | 1.00 | 50.63 | N |
| ATOM | 16914 | CZ | ARG | C | 307 | 38.946 | 17.609 | 43.939 | 1.00 | 47.81 | C |
| ATOM | 16915 | NH1 | ARG | C | 307 | 40.080 | 17.545 | 43.245 | 1.00 | 43.86 | N |
| ATOM | 16918 | NH2 | ARG | C | 307 | 38.217 | 16.525 | 44.053 | 1.00 | 50.54 | N |
| ATOM | 16921 | C | ARG | C | 307 | 38.629 | 21.735 | 40.241 | 1.00 | 58.15 | C |
| ATOM | 16922 | O | ARG | C | 307 | 38.176 | 20.778 | 39.624 | 1.00 | 66.15 | O |
| ATOM | 16923 | N | PRO | C | 308 | 39.755 | 22.318 | 39.870 | 1.00 | 59.79 | N |
| ATOM | 16924 | CA | PRO | C | 308 | 40.546 | 21.788 | 38.744 | 1.00 | 59.10 | C |
| ATOM | 16926 | CB | PRO | C | 308 | 41.640 | 22.856 | 38.523 | 1.00 | 56.70 | C |
| ATOM | 16929 | CG | PRO | C | 308 | 41.396 | 23.964 | 39.500 | 1.00 | 56.84 | C |
| ATOM | 16932 | CD | PRO | C | 308 | 40.358 | 23.515 | 40.488 | 1.00 | 59.81 | C |
| ATOM | 16935 | C | PRO | C | 308 | 41.210 | 20.429 | 39.057 | 1.00 | 57.82 | C |
| ATOM | 16936 | O | PRO | C | 308 | 41.842 | 20.326 | 40.111 | 1.00 | 54.21 | O |
| ATOM | 16937 | N | VAL | C | 309 | 41.077 | 19.441 | 38.160 | 1.00 | 58.91 | N |
| ATOM | 16939 | CA | VAL | C | 309 | 41.652 | 18.095 | 38.319 | 1.00 | 63.76 | C |
| ATOM | 16941 | CB | VAL | C | 309 | 40.551 | 17.114 | 38.722 | 1.00 | 65.79 | C |
| ATOM | 16943 | CG1 | VAL | C | 309 | 39.588 | 17.770 | 39.716 | 1.00 | 66.84 | C |
| ATOM | 16947 | CG2 | VAL | C | 309 | 39.779 | 16.616 | 37.484 | 1.00 | 67.52 | C |
| ATOM | 16951 | C | VAL | C | 309 | 42.356 | 17.527 | 37.044 | 1.00 | 69.36 | C |
| ATOM | 16952 | O | VAL | C | 309 | 42.656 | 18.262 | 36.125 | 1.00 | 69.68 | O |
| ATOM | 16953 | N | GLU | C | 310 | 42.636 | 16.222 | 37.004 | 1.00 | 77.12 | N |
| ATOM | 16955 | CA | GLU | C | 310 | 43.239 | 15.577 | 35.811 | 1.00 | 82.03 | C |
| ATOM | 16957 | CB | GLU | C | 310 | 44.374 | 14.638 | 36.233 | 1.00 | 81.97 | C |
| ATOM | 16960 | CG | GLU | C | 310 | 45.621 | 15.401 | 36.593 | 1.00 | 81.89 | C |
| ATOM | 16963 | CD | GLU | C | 310 | 45.544 | 16.821 | 36.063 | 1.00 | 80.73 | C |
| ATOM | 16964 | OE1 | GLU | C | 310 | 45.101 | 17.697 | 36.831 | 1.00 | 76.33 | O |
| ATOM | 16965 | OE2 | GLU | C | 310 | 45.908 | 17.048 | 34.889 | 1.00 | 75.15 | O |
| ATOM | 16966 | C | GLU | C | 310 | 42.302 | 14.768 | 34.903 | 1.00 | 87.34 | C |
| ATOM | 16967 | O | GLU | C | 310 | 41.793 | 13.708 | 35.306 | 1.00 | 84.75 | O |
| ATOM | 16968 | N | ASP | C | 311 | 42.089 | 15.239 | 33.670 | 1.00 | 93.03 | N |
| ATOM | 16970 | CA | ASP | C | 311 | 41.249 | 14.483 | 32.746 | 1.00 | 97.25 | C |
| ATOM | 16972 | CB | ASP | C | 311 | 41.259 | 15.044 | 31.311 | 1.00 | 99.29 | C |
| ATOM | 16975 | CG | ASP | C | 311 | 40.444 | 14.163 | 30.329 | 1.00 | 101.77 | C |
| ATOM | 16976 | OD1 | ASP | C | 311 | 40.021 | 13.036 | 30.712 | 1.00 | 101.63 | O |
| ATOM | 16977 | OD2 | ASP | C | 311 | 40.179 | 14.507 | 29.156 | 1.00 | 96.73 | O |
| ATOM | 16978 | C | ASP | C | 311 | 41.744 | 13.036 | 32.777 | 1.00 | 98.09 | C |
| ATOM | 16979 | O | ASP | C | 311 | 42.917 | 12.770 | 33.052 | 1.00 | 98.48 | O |
| ATOM | 16980 | N | VAL | C | 312 | 40.840 | 12.109 | 32.493 | 1.00 | 100.23 | N |
| ATOM | 16982 | CA | VAL | C | 312 | 41.132 | 10.686 | 32.624 | 1.00 | 101.33 | C |
| ATOM | 16984 | CB | VAL | C | 312 | 39.948 | 9.830 | 32.120 | 1.00 | 101.66 | C |
| ATOM | 16986 | CG1 | VAL | C | 312 | 40.264 | 8.348 | 32.290 | 1.00 | 102.68 | C |
| ATOM | 16990 | CG2 | VAL | C | 312 | 38.650 | 10.202 | 32.861 | 1.00 | 100.40 | C |
| ATOM | 16994 | C | VAL | C | 312 | 42.428 | 10.292 | 31.906 | 1.00 | 101.68 | C |
| ATOM | 16995 | O | VAL | C | 312 | 43.435 | 9.972 | 32.560 | 1.00 | 102.27 | O |
| ATOM | 16996 | N | ALA | C | 313 | 42.397 | 10.320 | 30.571 | 1.00 | 99.43 | N |
| ATOM | 16998 | CA | ALA | C | 313 | 43.564 | 9.986 | 29.760 | 1.00 | 96.81 | C |
| ATOM | 17000 | CB | ALA | C | 313 | 43.127 | 9.454 | 28.395 | 1.00 | 95.91 | C |
| ATOM | 17004 | C | ALA | C | 313 | 44.444 | 11.222 | 29.625 | 1.00 | 96.79 | C |
| ATOM | 17005 | O | ALA | C | 313 | 45.096 | 11.442 | 28.606 | 1.00 | 98.26 | O |
| ATOM | 17006 | N | THR | C | 314 | 44.434 | 12.009 | 30.703 | 1.00 | 97.05 | N |
| ATOM | 17008 | CA | THR | C | 314 | 45.147 | 13.297 | 30.885 | 1.00 | 96.61 | C |
| ATOM | 17010 | CB | THR | C | 314 | 46.635 | 12.993 | 31.175 | 1.00 | 95.85 | C |
| ATOM | 17012 | OG1 | THR | C | 314 | 47.210 | 12.241 | 30.101 | 1.00 | 94.24 | O |
| ATOM | 17014 | CG2 | THR | C | 314 | 46.755 | 12.067 | 32.397 | 1.00 | 97.66 | C |
| ATOM | 17018 | C | THR | C | 314 | 45.031 | 14.507 | 29.891 | 1.00 | 97.32 | C |
| ATOM | 17019 | O | THR | C | 314 | 45.827 | 15.447 | 29.999 | 1.00 | 99.24 | O |
| ATOM | 17020 | N | SER | C | 315 | 44.056 | 14.526 | 28.976 | 1.00 | 94.94 | N |
| ATOM | 17022 | CA | SER | C | 315 | 43.911 | 15.601 | 27.933 | 1.00 | 94.95 | C |
| ATOM | 17024 | CB | SER | C | 315 | 42.513 | 15.511 | 27.298 | 1.00 | 94.81 | C |
| ATOM | 17027 | OG | SER | C | 315 | 42.361 | 16.442 | 26.233 | 1.00 | 93.24 | O |
| ATOM | 17029 | C | SER | C | 315 | 44.158 | 17.114 | 28.237 | 1.00 | 93.29 | C |
| ATOM | 17030 | O | SER | C | 315 | 44.341 | 17.532 | 29.385 | 1.00 | 90.39 | O |
| ATOM | 17031 | N | GLN | C | 316 | 44.140 | 17.903 | 27.154 | 1.00 | 92.77 | N |
| ATOM | 17033 | CA | GLN | C | 316 | 44.289 | 19.362 | 27.190 | 1.00 | 93.00 | C |
| ATOM | 17035 | CB | GLN | C | 316 | 45.171 | 19.847 | 26.011 | 1.00 | 91.78 | C |
| ATOM | 17038 | CG | GLN | C | 316 | 46.679 | 19.416 | 26.098 | 1.00 | 93.54 | C |
| ATOM | 17041 | CD | GLN | C | 316 | 47.522 | 19.699 | 24.810 | 1.00 | 93.70 | C |
| ATOM | 17042 | OE1 | GLN | C | 316 | 47.456 | 20.789 | 24.226 | 1.00 | 91.79 | O |
| ATOM | 17043 | NE2 | GLN | C | 316 | 48.314 | 18.712 | 24.392 | 1.00 | 88.27 | N |
| ATOM | 17046 | C | GLN | C | 316 | 42.911 | 20.123 | 27.252 | 1.00 | 92.97 | C |
| ATOM | 17047 | O | GLN | C | 316 | 42.855 | 21.333 | 27.012 | 1.00 | 97.12 | O |
| ATOM | 17048 | N | ASP | C | 317 | 41.813 | 19.414 | 27.541 | 1.00 | 88.86 | N |
| ATOM | 17050 | CA | ASP | C | 317 | 40.545 | 20.057 | 27.925 | 1.00 | 85.72 | C |
| ATOM | 17052 | CB | ASP | C | 317 | 39.341 | 19.120 | 27.813 | 1.00 | 89.42 | C |
| ATOM | 17055 | CG | ASP | C | 317 | 39.375 | 18.237 | 26.588 | 1.00 | 93.20 | C |
| ATOM | 17056 | OD1 | ASP | C | 317 | 39.976 | 18.629 | 25.560 | 1.00 | 97.12 | O |
| ATOM | 17057 | OD2 | ASP | C | 317 | 38.806 | 17.125 | 26.583 | 1.00 | 93.67 | O |
| ATOM | 17058 | C | ASP | C | 317 | 40.650 | 20.378 | 29.414 | 1.00 | 81.00 | C |
| ATOM | 17059 | O | ASP | C | 317 | 41.068 | 19.527 | 30.190 | 1.00 | 76.59 | O |
| ATOM | 17060 | N | ASP | C | 318 | 40.260 | 21.571 | 29.843 | 1.00 | 76.29 | N |
| ATOM | 17062 | CA | ASP | C | 318 | 40.389 | 21.894 | 31.257 | 1.00 | 69.79 | C |
| ATOM | 17064 | CB | ASP | C | 318 | 40.429 | 23.405 | 31.434 | 1.00 | 70.73 | C |
| ATOM | 17067 | CG | ASP | C | 318 | 41.656 | 24.001 | 30.798 | 1.00 | 75.33 | C |
| ATOM | 17068 | OD1 | ASP | C | 318 | 41.776 | 25.257 | 30.699 | 1.00 | 80.77 | O |
| ATOM | 17069 | OD2 | ASP | C | 318 | 42.561 | 23.260 | 30.360 | 1.00 | 79.35 | O |
| ATOM | 17070 | C | ASP | C | 318 | 39.268 | 21.199 | 32.037 | 1.00 | 63.07 | C |
| ATOM | 17071 | O | ASP | C | 318 | 38.103 | 21.409 | 31.755 | 1.00 | 54.32 | O |
| ATOM | 17072 | N | CYS | C | 319 | 39.624 | 20.356 | 33.005 | 1.00 | 61.35 | N |
| ATOM | 17074 | CA | CYS | C | 319 | 38.611 | 19.604 | 33.753 | 1.00 | 62.73 | C |
| ATOM | 17076 | CB | CYS | C | 319 | 39.002 | 18.138 | 33.789 | 1.00 | 67.00 | C |
| ATOM | 17079 | SG | CYS | C | 319 | 39.025 | 17.347 | 32.147 | 1.00 | 71.83 | S |
| ATOM | 17080 | C | CYS | C | 319 | 38.309 | 20.122 | 35.176 | 1.00 | 60.27 | C |
| ATOM | 17081 | O | CYS | C | 319 | 39.157 | 20.741 | 35.825 | 1.00 | 57.82 | O |
| ATOM | 17082 | N | TYR | C | 320 | 37.089 | 19.870 | 35.652 | 1.00 | 53.68 | N |
| ATOM | 17084 | CA | TYR | C | 320 | 36.675 | 20.393 | 36.939 | 1.00 | 49.77 | C |
| ATOM | 17086 | CB | TYR | C | 320 | 35.945 | 21.709 | 36.677 | 1.00 | 50.01 | C |
| ATOM | 17089 | CG | TYR | C | 320 | 36.861 | 22.674 | 35.958 | 1.00 | 48.43 | C |
| ATOM | 17090 | CD1 | TYR | C | 320 | 36.774 | 22.878 | 34.601 | 1.00 | 45.90 | C |
| ATOM | 17092 | CE1 | TYR | C | 320 | 37.633 | 23.740 | 33.956 | 1.00 | 45.11 | C |
| ATOM | 17094 | CZ | TYR | C | 320 | 38.584 | 24.406 | 34.665 | 1.00 | 42.05 | C |
| ATOM | 17095 | OH | TYR | C | 320 | 39.464 | 25.286 | 34.063 | 1.00 | 41.52 | O |
| ATOM | 17097 | CE2 | TYR | C | 320 | 38.678 | 24.213 | 36.008 | 1.00 | 49.88 | C |
| ATOM | 17099 | CD2 | TYR | C | 320 | 37.824 | 23.357 | 36.645 | 1.00 | 48.03 | C |
| ATOM | 17101 | C | TYR | C | 320 | 35.796 | 19.427 | 37.713 | 1.00 | 50.65 | C |
| ATOM | 17102 | O | TYR | C | 320 | 35.243 | 18.517 | 37.104 | 1.00 | 50.64 | O |
| ATOM | 17103 | N | LYS | C | 321 | 35.681 | 19.611 | 39.042 | 1.00 | 48.99 | N |
| ATOM | 17105 | CA | LYS | C | 321 | 34.718 | 18.851 | 39.874 | 1.00 | 48.57 | C |
| ATOM | 17107 | CB | LYS | C | 321 | 35.394 | 17.846 | 40.803 | 1.00 | 51.22 | C |
| ATOM | 17110 | CG | LYS | C | 321 | 35.994 | 16.592 | 40.130 | 1.00 | 59.14 | C |
| ATOM | 17113 | CD | LYS | C | 321 | 36.034 | 15.363 | 41.092 | 1.00 | 60.36 | C |
| ATOM | 17116 | CE | LYS | C | 321 | 36.528 | 14.083 | 40.379 | 1.00 | 61.02 | C |
| ATOM | 17119 | NZ | LYS | C | 321 | 36.887 | 12.972 | 41.327 | 1.00 | 60.51 | N |
| ATOM | 17123 | C | LYS | C | 321 | 33.894 | 19.787 | 40.756 | 1.00 | 43.89 | C |
| ATOM | 17124 | O | LYS | C | 321 | 34.388 | 20.685 | 41.391 | 1.00 | 48.32 | O |
| ATOM | 17125 | N | PHE | C | 322 | 32.614 | 19.570 | 40.806 | 1.00 | 45.71 | N |
| ATOM | 17127 | CA | PHE | C | 322 | 31.772 | 20.381 | 41.647 | 1.00 | 45.11 | C |
| ATOM | 17129 | CB | PHE | C | 322 | 30.325 | 19.886 | 41.516 | 1.00 | 39.38 | C |
| ATOM | 17132 | CG | PHE | C | 322 | 29.275 | 20.890 | 41.937 | 1.00 | 47.52 | C |
| ATOM | 17133 | CD1 | PHE | C | 322 | 29.280 | 22.186 | 41.454 | 1.00 | 52.42 | C |
| ATOM | 17135 | CE1 | PHE | C | 322 | 28.318 | 23.066 | 41.839 | 1.00 | 46.38 | C |
| ATOM | 17137 | CZ | PHE | C | 322 | 27.334 | 22.671 | 42.708 | 1.00 | 41.85 | C |
| ATOM | 17139 | CE2 | PHE | C | 322 | 27.315 | 21.436 | 43.176 | 1.00 | 38.98 | C |
| ATOM | 17141 | CD2 | PHE | C | 322 | 28.266 | 20.535 | 42.801 | 1.00 | 38.92 | C |
| ATOM | 17143 | C | PHE | C | 322 | 32.331 | 20.143 | 43.041 | 1.00 | 46.56 | C |
| ATOM | 17144 | O | PHE | C | 322 | 32.334 | 18.998 | 43.496 | 1.00 | 55.34 | O |
| ATOM | 17145 | N | ALA | C | 323 | 32.827 | 21.180 | 43.713 | 1.00 | 40.68 | N |
| ATOM | 17147 | CA | ALA | C | 323 | 33.267 | 21.030 | 45.100 | 1.00 | 36.91 | C |
| ATOM | 17149 | CB | ALA | C | 323 | 34.573 | 21.721 | 45.298 | 1.00 | 40.42 | C |
| ATOM | 17153 | C | ALA | C | 323 | 32.266 | 21.542 | 46.125 | 1.00 | 36.58 | C |
| ATOM | 17154 | O | ALA | C | 323 | 32.636 | 22.208 | 47.080 | 1.00 | 37.20 | O |
| ATOM | 17155 | N | ILE | C | 324 | 30.989 | 21.267 | 45.943 | 1.00 | 37.33 | N |
| ATOM | 17157 | CA | ILE | C | 324 | 30.015 | 21.630 | 46.965 | 1.00 | 36.19 | C |
| ATOM | 17159 | CB | ILE | C | 324 | 29.023 | 22.599 | 46.424 | 1.00 | 39.76 | C |
| ATOM | 17161 | CG1 | ILE | C | 324 | 29.739 | 23.761 | 45.817 | 1.00 | 38.88 | C |
| ATOM | 17164 | CD1 | ILE | C | 324 | 28.812 | 24.873 | 45.598 | 1.00 | 45.35 | C |
| ATOM | 17168 | CG2 | ILE | C | 324 | 28.083 | 23.152 | 47.527 | 1.00 | 42.74 | C |
| ATOM | 17172 | C | ILE | C | 324 | 29.353 | 20.321 | 47.267 | 1.00 | 41.52 | C |
| ATOM | 17173 | O | ILE | C | 324 | 29.381 | 19.453 | 46.389 | 1.00 | 48.29 | O |
| ATOM | 17174 | N | SER | C | 325 | 28.788 | 20.145 | 48.473 | 1.00 | 43.90 | N |
| ATOM | 17176 | CA | SER | C | 325 | 28.161 | 18.864 | 48.868 | 1.00 | 44.02 | C |
| ATOM | 17178 | CB | SER | C | 325 | 29.154 | 17.696 | 48.881 | 1.00 | 45.54 | C |
| ATOM | 17181 | OG | SER | C | 325 | 30.435 | 18.073 | 49.334 | 1.00 | 56.81 | O |
| ATOM | 17183 | C | SER | C | 325 | 27.410 | 18.846 | 50.181 | 1.00 | 49.43 | C |
| ATOM | 17184 | O | SER | C | 325 | 27.528 | 19.750 | 51.015 | 1.00 | 54.04 | O |
| ATOM | 17185 | N | GLN | C | 326 | 26.624 | 17.786 | 50.339 | 1.00 | 55.37 | N |
| ATOM | 17187 | CA | GLN | C | 326 | 25.797 | 17.593 | 51.509 | 1.00 | 59.34 | C |
| ATOM | 17189 | CB | GLN | C | 326 | 24.512 | 16.803 | 51.166 | 1.00 | 62.25 | C |
| ATOM | 17192 | CG | GLN | C | 326 | 24.703 | 15.342 | 50.735 | 1.00 | 66.34 | C |
| ATOM | 17195 | CD | GLN | C | 326 | 23.421 | 14.731 | 50.154 | 1.00 | 73.31 | C |
| ATOM | 17196 | OE1 | GLN | C | 326 | 22.481 | 14.464 | 50.899 | 1.00 | 75.42 | O |
| ATOM | 17197 | NE2 | GLN | C | 326 | 23.388 | 14.511 | 48.826 | 1.00 | 73.17 | N |
| ATOM | 17200 | C | GLN | C | 326 | 26.568 | 16.965 | 52.682 | 1.00 | 60.65 | C |
| ATOM | 17201 | O | GLN | C | 326 | 27.603 | 16.287 | 52.522 | 1.00 | 58.34 | O |
| ATOM | 17202 | N | SER | C | 327 | 26.017 | 17.211 | 53.862 | 1.00 | 60.79 | N |
| ATOM | 17204 | CA | SER | C | 327 | 26.599 | 16.804 | 55.118 | 1.00 | 58.82 | C |
| ATOM | 17206 | CB | SER | C | 327 | 27.625 | 17.830 | 55.574 | 1.00 | 59.51 | C |
| ATOM | 17209 | OG | SER | C | 327 | 27.685 | 17.900 | 57.003 | 1.00 | 64.04 | O |
| ATOM | 17211 | C | SER | C | 327 | 25.482 | 16.698 | 56.153 | 1.00 | 58.47 | C |
| ATOM | 17212 | O | SER | C | 327 | 24.390 | 17.271 | 56.006 | 1.00 | 55.40 | O |
| ATOM | 17213 | N | SER | C | 328 | 25.771 | 15.949 | 57.197 | 1.00 | 56.87 | N |
| ATOM | 17215 | CA | SER | C | 328 | 24.812 | 15.697 | 58.242 | 1.00 | 55.69 | C |
| ATOM | 17217 | CB | SER | C | 328 | 24.601 | 14.209 | 58.396 | 1.00 | 56.03 | C |
| ATOM | 17220 | OG | SER | C | 328 | 25.854 | 13.578 | 58.688 | 1.00 | 57.06 | O |
| ATOM | 17222 | C | SER | C | 328 | 25.423 | 16.268 | 59.490 | 1.00 | 57.40 | C |
| ATOM | 17223 | O | SER | C | 328 | 24.834 | 16.198 | 60.576 | 1.00 | 60.77 | O |
| ATOM | 17224 | N | THR | C | 329 | 26.615 | 16.839 | 59.341 | 1.00 | 53.35 | N |
| ATOM | 17226 | CA | THR | C | 329 | 27.213 | 17.541 | 60.450 | 1.00 | 51.27 | C |
| ATOM | 17228 | CB | THR | C | 329 | 28.454 | 16.818 | 60.897 | 1.00 | 51.43 | C |
| ATOM | 17230 | OG1 | THR | C | 329 | 29.278 | 16.560 | 59.768 | 1.00 | 42.60 | O |
| ATOM | 17232 | CG2 | THR | C | 329 | 28.078 | 15.427 | 61.365 | 1.00 | 56.80 | C |
| ATOM | 17236 | C | THR | C | 329 | 27.466 | 19.007 | 60.149 | 1.00 | 48.82 | C |
| ATOM | 17237 | O | THR | C | 329 | 28.537 | 19.544 | 60.411 | 1.00 | 59.16 | O |
| ATOM | 17238 | N | GLY | C | 330 | 26.450 | 19.642 | 59.581 | 1.00 | 46.96 | N |
| ATOM | 17240 | CA | GLY | C | 330 | 26.372 | 21.083 | 59.478 | 1.00 | 34.71 | C |
| ATOM | 17243 | C | GLY | C | 330 | 27.118 | 21.538 | 58.279 | 1.00 | 32.91 | C |
| ATOM | 17244 | O | GLY | C | 330 | 27.478 | 20.707 | 57.498 | 1.00 | 31.32 | O |
| ATOM | 17245 | N | THR | C | 331 | 27.331 | 22.847 | 58.173 | 1.00 | 38.41 | N |
| ATOM | 17247 | CA | THR | C | 331 | 28.081 | 23.522 | 57.123 | 1.00 | 40.14 | C |
| ATOM | 17249 | CB | THR | C | 331 | 27.719 | 25.039 | 57.137 | 1.00 | 44.49 | C |
| ATOM | 17251 | OG1 | THR | C | 331 | 26.332 | 25.263 | 56.874 | 1.00 | 47.97 | O |
| ATOM | 17253 | CG2 | THR | C | 331 | 28.457 | 25.793 | 56.054 | 1.00 | 45.87 | C |
| ATOM | 17257 | C | THR | C | 331 | 29.560 | 23.524 | 57.507 | 1.00 | 43.45 | C |
| ATOM | 17258 | O | THR | C | 331 | 29.904 | 23.749 | 58.675 | 1.00 | 41.19 | O |
| ATOM | 17259 | N | VAL | C | 332 | 30.416 | 23.306 | 56.515 | 1.00 | 39.96 | N |
| ATOM | 17261 | CA | VAL | C | 332 | 31.828 | 23.320 | 56.680 | 1.00 | 35.05 | C |
| ATOM | 17263 | CB | VAL | C | 332 | 32.397 | 21.988 | 56.442 | 1.00 | 34.40 | C |
| ATOM | 17265 | CG1 | VAL | C | 332 | 33.907 | 22.017 | 56.732 | 1.00 | 32.26 | C |
| ATOM | 17269 | CG2 | VAL | C | 332 | 31.704 | 21.009 | 57.307 | 1.00 | 35.49 | C |
| ATOM | 17273 | C | VAL | C | 332 | 32.330 | 24.187 | 55.583 | 1.00 | 41.57 | C |
| ATOM | 17274 | O | VAL | C | 332 | 32.169 | 23.850 | 54.413 | 1.00 | 41.46 | O |
| ATOM | 17275 | N | MET | C | 333 | 32.918 | 25.314 | 55.956 | 1.00 | 43.32 | N |
| ATOM | 17277 | CA | MET | C | 333 | 33.419 | 26.236 | 54.977 | 1.00 | 46.33 | C |
| ATOM | 17279 | CB | MET | C | 333 | 33.426 | 27.663 | 55.549 | 1.00 | 48.59 | C |
| ATOM | 17282 | CG | MET | C | 333 | 32.239 | 28.470 | 55.227 | 1.00 | 48.65 | C |
| ATOM | 17285 | SD | MET | C | 333 | 31.907 | 29.702 | 56.521 | 1.00 | 59.78 | S |
| ATOM | 17286 | CE | MET | C | 333 | 32.913 | 30.942 | 55.931 | 1.00 | 59.17 | C |
| ATOM | 17290 | C | MET | C | 333 | 34.832 | 25.758 | 54.612 | 1.00 | 48.03 | C |
| ATOM | 17291 | O | MET | C | 333 | 35.809 | 26.193 | 55.201 | 1.00 | 42.88 | O |
| ATOM | 17292 | N | GLY | C | 334 | 34.915 | 24.862 | 53.635 | 1.00 | 48.50 | N |
| ATOM | 17294 | CA | GLY | C | 334 | 36.160 | 24.240 | 53.247 | 1.00 | 52.01 | C |
| ATOM | 17297 | C | GLY | C | 334 | 37.161 | 25.091 | 52.503 | 1.00 | 53.89 | C |
| ATOM | 17298 | O | GLY | C | 334 | 37.081 | 26.315 | 52.511 | 1.00 | 58.04 | O |
| ATOM | 17299 | N | ALA | C | 335 | 38.112 | 24.424 | 51.850 | 1.00 | 51.10 | N |
| ATOM | 17301 | CA | ALA | C | 335 | 39.176 | 25.126 | 51.170 | 1.00 | 50.84 | C |
| ATOM | 17303 | CB | ALA | C | 335 | 40.226 | 24.164 | 50.669 | 1.00 | 51.11 | C |
| ATOM | 17307 | C | ALA | C | 335 | 38.671 | 25.963 | 50.024 | 1.00 | 53.88 | C |
| ATOM | 17308 | O | ALA | C | 335 | 39.280 | 26.975 | 49.655 | 1.00 | 58.46 | O |
| ATOM | 17309 | N | VAL | C | 336 | 37.566 | 25.592 | 49.424 | 1.00 | 52.66 | N |
| ATOM | 17311 | CA | VAL | C | 336 | 37.223 | 26.366 | 48.255 | 1.00 | 56.89 | C |
| ATOM | 17313 | CB | VAL | C | 336 | 36.226 | 25.654 | 47.316 | 1.00 | 57.19 | C |
| ATOM | 17315 | CG1 | VAL | C | 336 | 36.745 | 24.245 | 46.966 | 1.00 | 56.50 | C |
| ATOM | 17319 | CG2 | VAL | C | 336 | 34.848 | 25.609 | 47.913 | 1.00 | 59.09 | C |
| ATOM | 17323 | C | VAL | C | 336 | 36.767 | 27.738 | 48.727 | 1.00 | 57.63 | C |
| ATOM | 17324 | O | VAL | C | 336 | 36.957 | 28.725 | 48.038 | 1.00 | 60.05 | O |
| ATOM | 17325 | N | ILE | C | 337 | 36.198 | 27.799 | 49.915 | 1.00 | 53.26 | N |
| ATOM | 17327 | CA | ILE | C | 337 | 35.714 | 29.050 | 50.421 | 1.00 | 54.88 | C |
| ATOM | 17329 | CB | ILE | C | 337 | 34.812 | 28.821 | 51.617 | 1.00 | 59.97 | C |
| ATOM | 17331 | CG1 | ILE | C | 337 | 33.605 | 28.021 | 51.167 | 1.00 | 62.66 | C |
| ATOM | 17334 | CD1 | ILE | C | 337 | 33.536 | 27.873 | 49.654 | 1.00 | 65.75 | C |
| ATOM | 17338 | CG2 | ILE | C | 337 | 34.416 | 30.142 | 52.257 | 1.00 | 61.20 | C |
| ATOM | 17342 | C | ILE | C | 337 | 36.884 | 29.853 | 50.861 | 1.00 | 55.72 | C |
| ATOM | 17343 | O | ILE | C | 337 | 36.885 | 31.076 | 50.754 | 1.00 | 60.34 | O |
| ATOM | 17344 | N | MET | C | 338 | 37.893 | 29.168 | 51.373 | 1.00 | 52.85 | N |
| ATOM | 17346 | CA | MET | C | 338 | 39.058 | 29.859 | 51.874 | 1.00 | 45.91 | C |
| ATOM | 17348 | CB | MET | C | 338 | 39.791 | 28.921 | 52.798 | 1.00 | 47.20 | C |
| ATOM | 17351 | CG | MET | C | 338 | 39.048 | 28.822 | 54.095 | 1.00 | 46.36 | C |
| ATOM | 17354 | SD | MET | C | 338 | 39.827 | 27.707 | 55.154 | 1.00 | 50.06 | S |
| ATOM | 17355 | CE | MET | C | 338 | 41.323 | 28.450 | 55.484 | 1.00 | 38.59 | C |
| ATOM | 17359 | C | MET | C | 338 | 39.946 | 30.388 | 50.744 | 1.00 | 42.52 | C |
| ATOM | 17360 | O | MET | C | 338 | 40.483 | 31.476 | 50.842 | 1.00 | 47.16 | O |
| ATOM | 17361 | N | GLU | C | 339 | 40.093 | 29.619 | 49.685 | 1.00 | 36.78 | N |
| ATOM | 17363 | CA | GLU | C | 339 | 40.815 | 30.034 | 48.514 | 1.00 | 37.17 | C |
| ATOM | 17365 | CB | GLU | C | 339 | 40.806 | 28.877 | 47.493 | 1.00 | 43.92 | C |
| ATOM | 17368 | CG | GLU | C | 339 | 41.688 | 27.679 | 47.897 | 1.00 | 48.10 | C |
| ATOM | 17371 | CD | GLU | C | 339 | 41.673 | 26.490 | 46.918 | 1.00 | 50.53 | C |
| ATOM | 17372 | OE1 | GLU | C | 339 | 42.111 | 25.367 | 47.340 | 1.00 | 46.87 | O |
| ATOM | 17373 | OE2 | GLU | C | 339 | 41.232 | 26.671 | 45.751 | 1.00 | 41.22 | O |
| ATOM | 17374 | C | GLU | C | 339 | 40.279 | 31.374 | 47.900 | 1.00 | 39.02 | C |
| ATOM | 17375 | O | GLU | C | 339 | 40.832 | 31.896 | 46.962 | 1.00 | 42.34 | O |
| ATOM | 17376 | N | GLY | C | 340 | 39.213 | 31.955 | 48.402 | 1.00 | 35.08 | N |
| ATOM | 17378 | CA | GLY | C | 340 | 38.963 | 33.297 | 47.960 | 1.00 | 33.46 | C |
| ATOM | 17381 | C | GLY | C | 340 | 39.436 | 34.371 | 48.950 | 1.00 | 36.86 | C |
| ATOM | 17382 | O | GLY | C | 340 | 39.491 | 35.574 | 48.678 | 1.00 | 33.13 | O |
| ATOM | 17383 | N | PHE | C | 341 | 39.800 | 33.978 | 50.139 | 1.00 | 35.16 | N |
| ATOM | 17385 | CA | PHE | C | 341 | 40.060 | 35.003 | 51.085 | 1.00 | 37.28 | C |
| ATOM | 17387 | CB | PHE | C | 341 | 38.892 | 34.974 | 52.025 | 1.00 | 38.32 | C |
| ATOM | 17390 | CG | PHE | C | 341 | 37.579 | 35.096 | 51.327 | 1.00 | 40.16 | C |
| ATOM | 17391 | CD1 | PHE | C | 341 | 36.751 | 33.993 | 51.165 | 1.00 | 41.87 | C |
| ATOM | 17393 | CE1 | PHE | C | 341 | 35.543 | 34.108 | 50.523 | 1.00 | 35.46 | C |
| ATOM | 17395 | CZ | PHE | C | 341 | 35.160 | 35.341 | 50.042 | 1.00 | 35.20 | C |
| ATOM | 17397 | CE2 | PHE | C | 341 | 35.976 | 36.453 | 50.198 | 1.00 | 27.10 | C |
| ATOM | 17399 | CD2 | PHE | C | 341 | 37.160 | 36.331 | 50.830 | 1.00 | 37.05 | C |
| ATOM | 17401 | C | PHE | C | 341 | 41.393 | 34.881 | 51.842 | 1.00 | 37.72 | C |
| ATOM | 17402 | O | PHE | C | 341 | 42.042 | 33.895 | 51.736 | 1.00 | 35.29 | O |
| ATOM | 17403 | N | TYR | C | 342 | 41.793 | 35.948 | 52.531 | 1.00 | 38.91 | N |
| ATOM | 17405 | CA | TYR | C | 342 | 42.864 | 35.934 | 53.519 | 1.00 | 40.66 | C |
| ATOM | 17407 | CB | TYR | C | 342 | 43.579 | 37.280 | 53.593 | 1.00 | 40.52 | C |
| ATOM | 17410 | CG | TYR | C | 342 | 44.730 | 37.363 | 54.555 | 1.00 | 39.32 | C |
| ATOM | 17411 | CD1 | TYR | C | 342 | 45.634 | 36.362 | 54.621 | 1.00 | 37.44 | C |
| ATOM | 17413 | CE1 | TYR | C | 342 | 46.694 | 36.415 | 55.487 | 1.00 | 39.89 | C |
| ATOM | 17415 | CZ | TYR | C | 342 | 46.880 | 37.469 | 56.308 | 1.00 | 40.51 | C |
| ATOM | 17416 | OH | TYR | C | 342 | 48.015 | 37.396 | 57.146 | 1.00 | 35.08 | O |
| ATOM | 17418 | CE2 | TYR | C | 342 | 45.972 | 38.527 | 56.273 | 1.00 | 33.09 | C |
| ATOM | 17420 | CD2 | TYR | C | 342 | 44.908 | 38.466 | 55.405 | 1.00 | 36.55 | C |
| ATOM | 17422 | C | TYR | C | 342 | 41.948 | 35.832 | 54.690 | 1.00 | 39.22 | C |
| ATOM | 17423 | O | TYR | C | 342 | 41.035 | 36.677 | 54.718 | 1.00 | 33.31 | O |
| ATOM | 17424 | N | VAL | C | 343 | 42.160 | 34.836 | 55.588 | 1.00 | 31.59 | N |
| ATOM | 17426 | CA | VAL | C | 343 | 41.279 | 34.548 | 56.731 | 1.00 | 32.15 | C |
| ATOM | 17428 | CB | VAL | C | 343 | 40.744 | 33.094 | 56.729 | 1.00 | 34.63 | C |
| ATOM | 17430 | CG1 | VAL | C | 343 | 39.678 | 32.894 | 57.800 | 1.00 | 37.79 | C |
| ATOM | 17434 | CG2 | VAL | C | 343 | 40.158 | 32.698 | 55.431 | 1.00 | 30.90 | C |
| ATOM | 17438 | C | VAL | C | 343 | 42.126 | 34.646 | 57.971 | 1.00 | 34.71 | C |
| ATOM | 17439 | O | VAL | C | 343 | 43.166 | 34.057 | 58.002 | 1.00 | 34.88 | O |
| ATOM | 17440 | N | VAL | C | 344 | 41.668 | 35.362 | 58.991 | 1.00 | 39.79 | N |
| ATOM | 17442 | CA | VAL | C | 344 | 42.422 | 35.621 | 60.206 | 1.00 | 38.00 | C |
| ATOM | 17444 | CB | VAL | C | 344 | 42.381 | 37.141 | 60.498 | 1.00 | 42.04 | C |
| ATOM | 17446 | CG1 | VAL | C | 344 | 42.852 | 37.469 | 61.904 | 1.00 | 48.20 | C |
| ATOM | 17450 | CG2 | VAL | C | 344 | 43.247 | 37.919 | 59.538 | 1.00 | 39.85 | C |
| ATOM | 17454 | C | VAL | C | 344 | 41.760 | 34.888 | 61.393 | 1.00 | 40.20 | C |
| ATOM | 17455 | O | VAL | C | 344 | 40.616 | 35.179 | 61.718 | 1.00 | 39.35 | O |
| ATOM | 17456 | N | PHE | C | 345 | 42.440 | 33.940 | 62.045 | 1.00 | 38.18 | N |
| ATOM | 17458 | CA | PHE | C | 345 | 41.849 | 33.277 | 63.219 | 1.00 | 38.67 | C |
| ATOM | 17460 | CB | PHE | C | 345 | 42.267 | 31.811 | 63.250 | 1.00 | 42.75 | C |
| ATOM | 17463 | CG | PHE | C | 345 | 41.608 | 30.985 | 62.172 | 1.00 | 44.70 | C |
| ATOM | 17464 | CD1 | PHE | C | 345 | 40.459 | 30.278 | 62.431 | 1.00 | 46.90 | C |
| ATOM | 17466 | CE1 | PHE | C | 345 | 39.861 | 29.540 | 61.441 | 1.00 | 45.20 | C |
| ATOM | 17468 | CZ | PHE | C | 345 | 40.403 | 29.522 | 60.202 | 1.00 | 37.04 | C |
| ATOM | 17470 | CE2 | PHE | C | 345 | 41.543 | 30.228 | 59.941 | 1.00 | 41.08 | C |
| ATOM | 17472 | CD2 | PHE | C | 345 | 42.133 | 30.944 | 60.899 | 1.00 | 40.32 | C |
| ATOM | 17474 | C | PHE | C | 345 | 42.193 | 34.007 | 64.536 | 1.00 | 37.35 | C |
| ATOM | 17475 | O | PHE | C | 345 | 43.045 | 33.621 | 65.279 | 1.00 | 41.94 | O |
| ATOM | 17476 | N | ASP | C | 346 | 41.504 | 35.077 | 64.834 | 1.00 | 39.98 | N |
| ATOM | 17478 | CA | ASP | C | 346 | 41.863 | 35.872 | 65.980 | 1.00 | 42.17 | C |
| ATOM | 17480 | CB | ASP | C | 346 | 41.330 | 37.273 | 65.805 | 1.00 | 45.99 | C |
| ATOM | 17483 | CG | ASP | C | 346 | 41.900 | 38.188 | 66.796 | 1.00 | 49.72 | C |
| ATOM | 17484 | OD1 | ASP | C | 346 | 42.136 | 37.663 | 67.899 | 1.00 | 52.14 | O |
| ATOM | 17485 | OD2 | ASP | C | 346 | 42.166 | 39.394 | 66.570 | 1.00 | 48.02 | O |
| ATOM | 17486 | C | ASP | C | 346 | 41.315 | 35.302 | 67.253 | 1.00 | 46.76 | C |
| ATOM | 17487 | O | ASP | C | 346 | 40.267 | 35.736 | 67.742 | 1.00 | 46.37 | O |
| ATOM | 17488 | N | ARG | C | 347 | 42.047 | 34.325 | 67.780 | 1.00 | 48.24 | N |
| ATOM | 17490 | CA | ARG | C | 347 | 41.697 | 33.614 | 68.997 | 1.00 | 47.24 | C |
| ATOM | 17492 | CB | ARG | C | 347 | 42.701 | 32.505 | 69.278 | 1.00 | 50.28 | C |
| ATOM | 17495 | CG | ARG | C | 347 | 42.704 | 31.426 | 68.259 | 1.00 | 51.78 | C |
| ATOM | 17498 | CD | ARG | C | 347 | 43.965 | 30.680 | 68.289 | 1.00 | 57.96 | C |
| ATOM | 17501 | NE | ARG | C | 347 | 44.139 | 30.045 | 69.591 | 1.00 | 62.86 | N |
| ATOM | 17503 | CZ | ARG | C | 347 | 43.999 | 28.748 | 69.803 | 1.00 | 58.59 | C |
| ATOM | 17504 | NH1 | ARG | C | 347 | 43.679 | 27.946 | 68.796 | 1.00 | 55.77 | N |
| ATOM | 17507 | NH2 | ARG | C | 347 | 44.176 | 28.254 | 71.026 | 1.00 | 57.83 | N |
| ATOM | 17510 | C | ARG | C | 347 | 41.666 | 34.492 | 70.205 | 1.00 | 47.41 | C |
| ATOM | 17511 | O | ARG | C | 347 | 40.776 | 34.334 | 71.022 | 1.00 | 48.83 | O |
| ATOM | 17512 | N | ALA | C | 348 | 42.633 | 35.393 | 70.342 | 1.00 | 48.64 | N |
| ATOM | 17514 | CA | ALA | C | 348 | 42.676 | 36.300 | 71.498 | 1.00 | 52.13 | C |
| ATOM | 17516 | CB | ALA | C | 348 | 43.786 | 37.298 | 71.352 | 1.00 | 55.88 | C |
| ATOM | 17520 | C | ALA | C | 348 | 41.368 | 37.045 | 71.671 | 1.00 | 55.01 | C |
| ATOM | 17521 | O | ALA | C | 348 | 40.819 | 37.132 | 72.778 | 1.00 | 57.03 | O |
| ATOM | 17522 | N | ARG | C | 349 | 40.864 | 37.571 | 70.563 | 1.00 | 55.66 | N |
| ATOM | 17524 | CA | ARG | C | 349 | 39.625 | 38.335 | 70.567 | 1.00 | 55.25 | C |
| ATOM | 17526 | CB | ARG | C | 349 | 39.834 | 39.524 | 69.656 | 1.00 | 56.27 | C |
| ATOM | 17529 | CG | ARG | C | 349 | 41.089 | 40.321 | 70.104 | 1.00 | 61.14 | C |
| ATOM | 17532 | CD | ARG | C | 349 | 41.529 | 41.415 | 69.160 | 1.00 | 61.41 | C |
| ATOM | 17535 | NE | ARG | C | 349 | 40.448 | 42.361 | 68.951 | 1.00 | 67.89 | N |
| ATOM | 17537 | CZ | ARG | C | 349 | 40.598 | 43.555 | 68.413 | 1.00 | 67.22 | C |
| ATOM | 17538 | NH1 | ARG | C | 349 | 41.799 | 43.941 | 68.019 | 1.00 | 57.57 | N |
| ATOM | 17541 | NH2 | ARG | C | 349 | 39.538 | 44.355 | 68.266 | 1.00 | 68.37 | N |
| ATOM | 17544 | C | ARG | C | 349 | 38.392 | 37.516 | 70.166 | 1.00 | 56.07 | C |
| ATOM | 17545 | O | ARG | C | 349 | 37.361 | 38.057 | 69.810 | 1.00 | 57.65 | O |
| ATOM | 17546 | N | LYS | C | 350 | 38.513 | 36.199 | 70.229 | 1.00 | 57.20 | N |
| ATOM | 17548 | CA | LYS | C | 350 | 37.426 | 35.311 | 69.892 | 1.00 | 55.54 | C |
| ATOM | 17550 | CB | LYS | C | 350 | 36.402 | 35.347 | 71.008 | 1.00 | 58.01 | C |
| ATOM | 17553 | CG | LYS | C | 350 | 35.244 | 34.383 | 70.795 | 1.00 | 63.30 | C |
| ATOM | 17556 | CD | LYS | C | 350 | 34.646 | 33.997 | 72.152 | 1.00 | 67.63 | C |
| ATOM | 17559 | CE | LYS | C | 350 | 34.093 | 32.572 | 72.190 | 1.00 | 67.21 | C |
| ATOM | 17562 | NZ | LYS | C | 350 | 33.300 | 32.324 | 73.459 | 1.00 | 66.54 | N |
| ATOM | 17566 | C | LYS | C | 350 | 36.758 | 35.693 | 68.595 | 1.00 | 54.63 | C |
| ATOM | 17567 | O | LYS | C | 350 | 35.541 | 35.923 | 68.575 | 1.00 | 58.43 | O |
| ATOM | 17568 | N | ARG | C | 351 | 37.520 | 35.768 | 67.506 | 1.00 | 51.33 | N |
| ATOM | 17570 | CA | ARG | C | 351 | 36.921 | 36.228 | 66.232 | 1.00 | 51.25 | C |
| ATOM | 17572 | CB | ARG | C | 351 | 36.660 | 37.752 | 66.270 | 1.00 | 49.27 | C |
| ATOM | 17575 | CG | ARG | C | 351 | 37.868 | 38.645 | 65.954 | 1.00 | 51.56 | C |
| ATOM | 17578 | CD | ARG | C | 351 | 37.696 | 40.138 | 66.363 | 1.00 | 54.35 | C |
| ATOM | 17581 | NE | ARG | C | 351 | 38.944 | 40.898 | 66.236 | 1.00 | 57.49 | N |
| ATOM | 17583 | CZ | ARG | C | 351 | 39.061 | 42.151 | 65.802 | 1.00 | 51.36 | C |
| ATOM | 17584 | NH1 | ARG | C | 351 | 38.005 | 42.835 | 65.434 | 1.00 | 51.03 | N |
| ATOM | 17587 | NH2 | ARG | C | 351 | 40.259 | 42.717 | 65.731 | 1.00 | 48.91 | N |
| ATOM | 17590 | C | ARG | C | 351 | 37.698 | 35.864 | 64.967 | 1.00 | 47.27 | C |
| ATOM | 17591 | O | ARG | C | 351 | 38.916 | 35.715 | 65.021 | 1.00 | 40.70 | O |
| ATOM | 17592 | N | ILE | C | 352 | 36.964 | 35.727 | 63.848 | 1.00 | 45.34 | N |
| ATOM | 17594 | CA | ILE | C | 352 | 37.525 | 35.396 | 62.525 | 1.00 | 43.43 | C |
| ATOM | 17596 | CB | ILE | C | 352 | 36.818 | 34.196 | 61.969 | 1.00 | 45.87 | C |
| ATOM | 17598 | CG1 | ILE | C | 352 | 36.949 | 33.036 | 62.953 | 1.00 | 46.35 | C |
| ATOM | 17601 | CD1 | ILE | C | 352 | 36.010 | 31.923 | 62.680 | 1.00 | 50.54 | C |
| ATOM | 17605 | CG2 | ILE | C | 352 | 37.425 | 33.812 | 60.638 | 1.00 | 46.09 | C |
| ATOM | 17609 | C | ILE | C | 352 | 37.427 | 36.552 | 61.493 | 1.00 | 44.39 | C |
| ATOM | 17610 | O | ILE | C | 352 | 36.374 | 37.193 | 61.332 | 1.00 | 40.87 | O |
| ATOM | 17611 | N | GLY | C | 353 | 38.530 | 36.806 | 60.787 | 1.00 | 43.24 | N |
| ATOM | 17613 | CA | GLY | C | 353 | 38.623 | 37.933 | 59.874 | 1.00 | 43.55 | C |
| ATOM | 17616 | C | GLY | C | 353 | 38.515 | 37.530 | 58.428 | 1.00 | 45.48 | C |
| ATOM | 17617 | O | GLY | C | 353 | 39.044 | 36.500 | 58.055 | 1.00 | 54.82 | O |
| ATOM | 17618 | N | PHE | C | 354 | 37.849 | 38.327 | 57.598 | 1.00 | 45.78 | N |
| ATOM | 17620 | CA | PHE | C | 354 | 37.715 | 37.991 | 56.179 | 1.00 | 40.77 | C |
| ATOM | 17622 | CB | PHE | C | 354 | 36.296 | 37.551 | 55.869 | 1.00 | 39.83 | C |
| ATOM | 17625 | CG | PHE | C | 354 | 35.966 | 36.142 | 56.295 | 1.00 | 35.75 | C |
| ATOM | 17626 | CD1 | PHE | C | 354 | 35.660 | 35.850 | 57.574 | 1.00 | 42.08 | C |
| ATOM | 17628 | CE1 | PHE | C | 354 | 35.352 | 34.571 | 57.950 | 1.00 | 32.98 | C |
| ATOM | 17630 | CZ | PHE | C | 354 | 35.355 | 33.598 | 57.059 | 1.00 | 29.90 | C |
| ATOM | 17632 | CE2 | PHE | C | 354 | 35.652 | 33.862 | 55.807 | 1.00 | 31.02 | C |
| ATOM | 17634 | CD2 | PHE | C | 354 | 35.952 | 35.131 | 55.416 | 1.00 | 35.30 | C |
| ATOM | 17636 | C | PHE | C | 354 | 38.083 | 39.200 | 55.295 | 1.00 | 41.72 | C |
| ATOM | 17637 | O | PHE | C | 354 | 37.714 | 40.315 | 55.585 | 1.00 | 34.33 | O |
| ATOM | 17638 | N | ALA | C | 355 | 38.824 | 38.935 | 54.221 | 1.00 | 43.10 | N |
| ATOM | 17640 | CA | ALA | C | 355 | 39.267 | 39.937 | 53.283 | 1.00 | 39.49 | C |
| ATOM | 17642 | CB | ALA | C | 355 | 40.493 | 40.651 | 53.791 | 1.00 | 46.03 | C |
| ATOM | 17646 | C | ALA | C | 355 | 39.622 | 39.226 | 52.013 | 1.00 | 41.04 | C |
| ATOM | 17647 | O | ALA | C | 355 | 40.040 | 38.078 | 52.026 | 1.00 | 42.04 | O |
| ATOM | 17648 | N | VAL | C | 356 | 39.464 | 39.934 | 50.910 | 1.00 | 41.73 | N |
| ATOM | 17650 | CA | VAL | C | 356 | 39.681 | 39.388 | 49.610 | 1.00 | 38.27 | C |
| ATOM | 17652 | CB | VAL | C | 356 | 39.294 | 40.438 | 48.541 | 1.00 | 40.02 | C |
| ATOM | 17654 | CG1 | VAL | C | 356 | 39.311 | 39.862 | 47.108 | 1.00 | 44.27 | C |
| ATOM | 17658 | CG2 | VAL | C | 356 | 37.936 | 40.971 | 48.802 | 1.00 | 31.45 | C |
| ATOM | 17662 | C | VAL | C | 356 | 41.156 | 39.105 | 49.654 | 1.00 | 44.04 | C |
| ATOM | 17663 | O | VAL | C | 356 | 41.903 | 39.922 | 50.189 | 1.00 | 46.65 | O |
| ATOM | 17664 | N | SER | C | 357 | 41.521 | 37.932 | 49.121 | 1.00 | 49.35 | N |
| ATOM | 17666 | CA | SER | C | 357 | 42.877 | 37.379 | 48.946 | 1.00 | 46.26 | C |
| ATOM | 17668 | CB | SER | C | 357 | 42.764 | 35.885 | 48.732 | 1.00 | 47.49 | C |
| ATOM | 17671 | OG | SER | C | 357 | 43.936 | 35.334 | 48.149 | 1.00 | 57.37 | O |
| ATOM | 17673 | C | SER | C | 357 | 43.563 | 37.880 | 47.719 | 1.00 | 48.33 | C |
| ATOM | 17674 | O | SER | C | 357 | 42.975 | 37.887 | 46.664 | 1.00 | 46.33 | O |
| ATOM | 17675 | N | ALA | C | 358 | 44.830 | 38.271 | 47.846 | 1.00 | 56.29 | N |
| ATOM | 17677 | CA | ALA | C | 358 | 45.583 | 38.848 | 46.722 | 1.00 | 56.23 | C |
| ATOM | 17679 | CB | ALA | C | 358 | 46.834 | 39.538 | 47.217 | 1.00 | 56.89 | C |
| ATOM | 17683 | C | ALA | C | 358 | 45.994 | 37.860 | 45.668 | 1.00 | 54.12 | C |
| ATOM | 17684 | O | ALA | C | 358 | 46.607 | 38.256 | 44.708 | 1.00 | 56.22 | O |
| ATOM | 17685 | N | CYS | C | 359 | 45.696 | 36.589 | 45.852 | 1.00 | 54.48 | N |
| ATOM | 17687 | CA | CYS | C | 359 | 46.053 | 35.583 | 44.874 | 1.00 | 58.48 | C |
| ATOM | 17689 | CB | CYS | C | 359 | 47.071 | 34.636 | 45.479 | 1.00 | 60.40 | C |
| ATOM | 17692 | SG | CYS | C | 359 | 46.287 | 33.673 | 46.804 | 1.00 | 70.62 | S |
| ATOM | 17693 | C | CYS | C | 359 | 44.866 | 34.740 | 44.441 | 1.00 | 55.69 | C |
| ATOM | 17694 | O | CYS | C | 359 | 45.065 | 33.670 | 43.872 | 1.00 | 58.26 | O |
| ATOM | 17695 | N | HIS | C | 360 | 43.646 | 35.200 | 44.698 | 1.00 | 52.56 | N |
| ATOM | 17697 | CA | HIS | C | 360 | 42.458 | 34.444 | 44.313 | 1.00 | 47.84 | C |
| ATOM | 17699 | CB | HIS | C | 360 | 41.228 | 34.911 | 45.062 | 1.00 | 44.33 | C |
| ATOM | 17702 | CG | HIS | C | 360 | 40.456 | 35.978 | 44.368 | 1.00 | 36.46 | C |
| ATOM | 17703 | ND1 | HIS | C | 360 | 40.527 | 37.304 | 44.749 | 1.00 | 35.80 | N |
| ATOM | 17705 | CE1 | HIS | C | 360 | 39.725 | 38.021 | 43.980 | 1.00 | 32.97 | C |
| ATOM | 17707 | NE2 | HIS | C | 360 | 39.146 | 37.205 | 43.108 | 1.00 | 35.99 | N |
| ATOM | 17709 | CD2 | HIS | C | 360 | 39.583 | 35.920 | 43.334 | 1.00 | 24.33 | C |
| ATOM | 17711 | C | HIS | C | 360 | 42.149 | 34.453 | 42.837 | 1.00 | 48.33 | C |
| ATOM | 17712 | O | HIS | C | 360 | 41.972 | 35.472 | 42.212 | 1.00 | 55.09 | O |
| ATOM | 17713 | N | VAL | C | 361 | 42.049 | 33.269 | 42.301 | 1.00 | 48.18 | N |
| ATOM | 17715 | CA | VAL | C | 361 | 41.775 | 33.081 | 40.913 | 1.00 | 49.01 | C |
| ATOM | 17717 | CB | VAL | C | 361 | 41.585 | 31.602 | 40.711 | 1.00 | 46.22 | C |
| ATOM | 17719 | CG1 | VAL | C | 361 | 41.177 | 31.321 | 39.315 | 1.00 | 51.10 | C |
| ATOM | 17723 | CG2 | VAL | C | 361 | 42.857 | 30.908 | 41.025 | 1.00 | 47.29 | C |
| ATOM | 17727 | C | VAL | C | 361 | 40.511 | 33.766 | 40.444 | 1.00 | 51.23 | C |
| ATOM | 17728 | O | VAL | C | 361 | 39.424 | 33.400 | 40.904 | 1.00 | 56.14 | O |
| ATOM | 17729 | N | HIS | C | 362 | 40.623 | 34.748 | 39.552 | 1.00 | 52.97 | N |
| ATOM | 17731 | CA | HIS | C | 362 | 39.418 | 35.335 | 38.940 | 1.00 | 57.75 | C |
| ATOM | 17733 | CB | HIS | C | 362 | 38.911 | 36.603 | 39.652 | 1.00 | 62.76 | C |
| ATOM | 17736 | CG | HIS | C | 362 | 39.899 | 37.726 | 39.721 | 1.00 | 66.49 | C |
| ATOM | 17737 | ND1 | HIS | C | 362 | 40.724 | 37.924 | 40.807 | 1.00 | 69.80 | N |
| ATOM | 17739 | CE1 | HIS | C | 362 | 41.485 | 38.984 | 40.598 | 1.00 | 70.34 | C |
| ATOM | 17741 | NE2 | HIS | C | 362 | 41.181 | 39.486 | 39.417 | 1.00 | 71.05 | N |
| ATOM | 17743 | CD2 | HIS | C | 362 | 40.187 | 38.721 | 38.849 | 1.00 | 71.53 | C |
| ATOM | 17745 | C | HIS | C | 362 | 39.569 | 35.585 | 37.467 | 1.00 | 56.16 | C |
| ATOM | 17746 | O | HIS | C | 362 | 40.553 | 35.203 | 36.886 | 1.00 | 55.35 | O |
| ATOM | 17747 | N | ASP | C | 363 | 38.559 | 36.210 | 36.869 | 1.00 | 62.61 | N |
| ATOM | 17749 | CA | ASP | C | 363 | 38.561 | 36.570 | 35.436 | 1.00 | 63.83 | C |
| ATOM | 17751 | CB | ASP | C | 363 | 37.395 | 35.908 | 34.686 | 1.00 | 62.33 | C |
| ATOM | 17754 | CG | ASP | C | 363 | 36.028 | 36.093 | 35.405 | 1.00 | 67.13 | C |
| ATOM | 17755 | OD1 | ASP | C | 363 | 35.956 | 36.855 | 36.408 | 1.00 | 60.86 | O |
| ATOM | 17756 | OD2 | ASP | C | 363 | 34.969 | 35.506 | 35.031 | 1.00 | 63.80 | O |
| ATOM | 17757 | C | ASP | C | 363 | 38.439 | 38.084 | 35.425 | 1.00 | 62.69 | C |
| ATOM | 17758 | O | ASP | C | 363 | 38.327 | 38.695 | 36.488 | 1.00 | 65.13 | O |
| ATOM | 17759 | N | GLU | C | 364 | 38.443 | 38.706 | 34.259 | 1.00 | 62.65 | N |
| ATOM | 17761 | CA | GLU | C | 364 | 38.410 | 40.178 | 34.212 | 1.00 | 60.57 | C |
| ATOM | 17763 | CB | GLU | C | 364 | 38.753 | 40.605 | 32.793 | 1.00 | 60.33 | C |
| ATOM | 17766 | CG | GLU | C | 364 | 38.194 | 41.926 | 32.333 | 1.00 | 61.49 | C |
| ATOM | 17769 | CD | GLU | C | 364 | 38.714 | 42.269 | 30.943 | 1.00 | 70.09 | C |
| ATOM | 17770 | OE1 | GLU | C | 364 | 38.632 | 41.370 | 30.055 | 1.00 | 68.45 | O |
| ATOM | 17771 | OE2 | GLU | C | 364 | 39.216 | 43.417 | 30.749 | 1.00 | 72.62 | O |
| ATOM | 17772 | C | GLU | C | 364 | 37.097 | 40.860 | 34.703 | 1.00 | 55.57 | C |
| ATOM | 17773 | O | GLU | C | 364 | 37.111 | 41.994 | 35.171 | 1.00 | 55.50 | O |
| ATOM | 17774 | N | PHE | C | 365 | 35.971 | 40.175 | 34.628 | 1.00 | 50.41 | N |
| ATOM | 17776 | CA | PHE | C | 365 | 34.702 | 40.825 | 34.944 | 1.00 | 48.24 | C |
| ATOM | 17778 | CB | PHE | C | 365 | 33.671 | 40.470 | 33.882 | 1.00 | 48.33 | C |
| ATOM | 17781 | CG | PHE | C | 365 | 34.219 | 40.334 | 32.501 | 1.00 | 48.95 | C |
| ATOM | 17782 | CD1 | PHE | C | 365 | 34.692 | 39.123 | 32.072 | 1.00 | 49.95 | C |
| ATOM | 17784 | CE1 | PHE | C | 365 | 35.199 | 38.950 | 30.789 | 1.00 | 53.08 | C |
| ATOM | 17786 | CZ | PHE | C | 365 | 35.233 | 39.991 | 29.912 | 1.00 | 55.82 | C |
| ATOM | 17788 | CE2 | PHE | C | 365 | 34.750 | 41.271 | 30.319 | 1.00 | 56.84 | C |
| ATOM | 17790 | CD2 | PHE | C | 365 | 34.243 | 41.424 | 31.616 | 1.00 | 56.84 | C |
| ATOM | 17792 | C | PHE | C | 365 | 34.054 | 40.497 | 36.292 | 1.00 | 45.78 | C |
| ATOM | 17793 | O | PHE | C | 365 | 32.976 | 40.993 | 36.576 | 1.00 | 49.51 | O |
| ATOM | 17794 | N | ARG | C | 366 | 34.682 | 39.663 | 37.105 | 1.00 | 45.57 | N |
| ATOM | 17796 | CA | ARG | C | 366 | 34.121 | 39.240 | 38.385 | 1.00 | 41.73 | C |
| ATOM | 17798 | CB | ARG | C | 366 | 33.243 | 38.013 | 38.194 | 1.00 | 38.80 | C |
| ATOM | 17801 | CG | ARG | C | 366 | 31.822 | 38.352 | 38.384 | 1.00 | 37.22 | C |
| ATOM | 17804 | CD | ARG | C | 366 | 30.802 | 37.311 | 38.128 | 1.00 | 40.56 | C |
| ATOM | 17807 | NE | ARG | C | 366 | 31.257 | 36.052 | 37.537 | 1.00 | 48.25 | N |
| ATOM | 17809 | CZ | ARG | C | 366 | 30.432 | 35.033 | 37.321 | 1.00 | 47.05 | C |
| ATOM | 17810 | NH1 | ARG | C | 366 | 29.168 | 35.161 | 37.641 | 1.00 | 44.28 | N |
| ATOM | 17813 | NH2 | ARG | C | 366 | 30.852 | 33.894 | 36.787 | 1.00 | 53.80 | N |
| ATOM | 17816 | C | ARG | C | 366 | 35.212 | 38.904 | 39.367 | 1.00 | 42.95 | C |
| ATOM | 17817 | O | ARG | C | 366 | 36.107 | 38.169 | 39.041 | 1.00 | 47.17 | O |
| ATOM | 17818 | N | THR | C | 367 | 35.105 | 39.427 | 40.577 | 1.00 | 45.66 | N |
| ATOM | 17820 | CA | THR | C | 367 | 36.140 | 39.279 | 41.588 | 1.00 | 47.80 | C |
| ATOM | 17822 | CB | THR | C | 367 | 36.868 | 40.622 | 41.749 | 1.00 | 51.39 | C |
| ATOM | 17824 | OG1 | THR | C | 367 | 38.131 | 40.423 | 42.363 | 1.00 | 57.38 | O |
| ATOM | 17826 | CG2 | THR | C | 367 | 36.156 | 41.538 | 42.774 | 1.00 | 56.23 | C |
| ATOM | 17830 | C | THR | C | 367 | 35.492 | 38.965 | 42.905 | 1.00 | 46.46 | C |
| ATOM | 17831 | O | THR | C | 367 | 34.522 | 39.642 | 43.277 | 1.00 | 54.55 | O |
| ATOM | 17832 | N | ALA | C | 368 | 35.993 | 37.977 | 43.640 | 1.00 | 39.88 | N |
| ATOM | 17834 | CA | ALA | C | 368 | 35.375 | 37.681 | 44.909 | 1.00 | 32.52 | C |
| ATOM | 17836 | CB | ALA | C | 368 | 36.048 | 36.619 | 45.526 | 1.00 | 37.31 | C |
| ATOM | 17840 | C | ALA | C | 368 | 35.389 | 38.935 | 45.813 | 1.00 | 39.83 | C |
| ATOM | 17841 | O | ALA | C | 368 | 36.237 | 39.822 | 45.723 | 1.00 | 44.69 | O |
| ATOM | 17842 | N | ALA | C | 369 | 34.440 | 39.005 | 46.715 | 1.00 | 43.60 | N |
| ATOM | 17844 | CA | ALA | C | 369 | 34.275 | 40.200 | 47.502 | 1.00 | 40.45 | C |
| ATOM | 17846 | CB | ALA | C | 369 | 33.188 | 40.996 | 46.899 | 1.00 | 43.87 | C |
| ATOM | 17850 | C | ALA | C | 369 | 33.937 | 39.926 | 48.946 | 1.00 | 37.91 | C |
| ATOM | 17851 | O | ALA | C | 369 | 33.672 | 38.782 | 49.352 | 1.00 | 37.55 | O |
| ATOM | 17852 | N | VAL | C | 370 | 33.960 | 41.017 | 49.694 | 1.00 | 36.68 | N |
| ATOM | 17854 | CA | VAL | C | 370 | 33.625 | 41.058 | 51.097 | 1.00 | 36.40 | C |
| ATOM | 17856 | CB | VAL | C | 370 | 34.810 | 40.835 | 51.938 | 1.00 | 34.82 | C |
| ATOM | 17858 | CG1 | VAL | C | 370 | 34.375 | 40.751 | 53.345 | 1.00 | 39.75 | C |
| ATOM | 17862 | CG2 | VAL | C | 370 | 35.455 | 39.559 | 51.558 | 1.00 | 40.98 | C |
| ATOM | 17866 | C | VAL | C | 370 | 33.125 | 42.479 | 51.368 | 1.00 | 38.45 | C |
| ATOM | 17867 | O | VAL | C | 370 | 33.886 | 43.400 | 51.320 | 1.00 | 26.92 | O |
| ATOM | 17868 | N | GLU | C | 371 | 31.840 | 42.634 | 51.674 | 1.00 | 44.21 | N |
| ATOM | 17870 | CA | GLU | C | 371 | 31.246 | 43.943 | 51.819 | 1.00 | 47.61 | C |
| ATOM | 17872 | CB | GLU | C | 371 | 30.441 | 44.204 | 50.559 | 1.00 | 51.18 | C |
| ATOM | 17875 | CG | GLU | C | 371 | 31.339 | 44.513 | 49.383 | 1.00 | 54.46 | C |
| ATOM | 17878 | CD | GLU | C | 371 | 30.714 | 44.152 | 48.063 | 1.00 | 57.83 | C |
| ATOM | 17879 | OE1 | GLU | C | 371 | 31.465 | 44.024 | 47.072 | 1.00 | 64.47 | O |
| ATOM | 17880 | OE2 | GLU | C | 371 | 29.479 | 43.997 | 48.016 | 1.00 | 63.16 | O |
| ATOM | 17881 | C | GLU | C | 371 | 30.337 | 44.077 | 53.026 | 1.00 | 48.24 | C |
| ATOM | 17882 | O | GLU | C | 371 | 29.893 | 43.081 | 53.576 | 1.00 | 47.20 | O |
| ATOM | 17883 | N | GLY | C | 372 | 30.062 | 45.311 | 53.440 | 1.00 | 50.29 | N |
| ATOM | 17885 | CA | GLY | C | 372 | 29.156 | 45.560 | 54.560 | 1.00 | 50.67 | C |
| ATOM | 17888 | C | GLY | C | 372 | 29.149 | 47.025 | 54.947 | 1.00 | 50.50 | C |
| ATOM | 17889 | O | GLY | C | 372 | 29.884 | 47.806 | 54.361 | 1.00 | 51.93 | O |
| ATOM | 17890 | N | PRO | C | 373 | 28.353 | 47.431 | 55.934 | 1.00 | 52.13 | N |
| ATOM | 17891 | CA | PRO | C | 373 | 27.466 | 46.568 | 56.733 | 1.00 | 47.64 | C |
| ATOM | 17893 | CB | PRO | C | 373 | 27.318 | 47.351 | 58.016 | 1.00 | 51.45 | C |
| ATOM | 17896 | CG | PRO | C | 373 | 27.992 | 48.699 | 57.790 | 1.00 | 49.65 | C |
| ATOM | 17899 | CD | PRO | C | 373 | 28.278 | 48.839 | 56.356 | 1.00 | 47.50 | C |
| ATOM | 17902 | C | PRO | C | 373 | 26.077 | 46.434 | 56.223 | 1.00 | 46.53 | C |
| ATOM | 17903 | O | PRO | C | 373 | 25.632 | 47.293 | 55.527 | 1.00 | 45.34 | O |
| ATOM | 17904 | N | PHE | C | 374 | 25.398 | 45.365 | 56.607 | 1.00 | 52.57 | N |
| ATOM | 17906 | CA | PHE | C | 374 | 24.028 | 45.097 | 56.195 | 1.00 | 50.90 | C |
| ATOM | 17908 | CB | PHE | C | 374 | 23.894 | 43.751 | 55.484 | 1.00 | 49.66 | C |
| ATOM | 17911 | CG | PHE | C | 374 | 24.437 | 43.797 | 54.115 | 1.00 | 48.09 | C |
| ATOM | 17912 | CD1 | PHE | C | 374 | 25.718 | 43.429 | 53.899 | 1.00 | 39.08 | C |
| ATOM | 17914 | CE1 | PHE | C | 374 | 26.255 | 43.495 | 52.658 | 1.00 | 42.90 | C |
| ATOM | 17916 | CZ | PHE | C | 374 | 25.507 | 43.947 | 51.602 | 1.00 | 42.40 | C |
| ATOM | 17918 | CE2 | PHE | C | 374 | 24.192 | 44.339 | 51.799 | 1.00 | 39.12 | C |
| ATOM | 17920 | CD2 | PHE | C | 374 | 23.660 | 44.272 | 53.042 | 1.00 | 42.78 | C |
| ATOM | 17922 | C | PHE | C | 374 | 23.203 | 45.056 | 57.387 | 1.00 | 52.02 | C |
| ATOM | 17923 | O | PHE | C | 374 | 23.499 | 44.353 | 58.298 | 1.00 | 54.34 | O |
| ATOM | 17924 | N | VAL | C | 375 | 22.141 | 45.825 | 57.369 | 1.00 | 64.41 | N |
| ATOM | 17926 | CA | VAL | C | 375 | 21.259 | 45.867 | 58.496 | 1.00 | 65.17 | C |
| ATOM | 17928 | CB | VAL | C | 375 | 20.319 | 47.035 | 58.465 | 1.00 | 65.29 | C |
| ATOM | 17930 | CG1 | VAL | C | 375 | 18.943 | 46.570 | 58.832 | 1.00 | 62.93 | C |
| ATOM | 17934 | CG2 | VAL | C | 375 | 20.850 | 48.116 | 59.416 | 1.00 | 68.23 | C |
| ATOM | 17938 | C | VAL | C | 375 | 20.504 | 44.604 | 58.423 | 1.00 | 65.98 | C |
| ATOM | 17939 | O | VAL | C | 375 | 19.770 | 44.346 | 57.465 | 1.00 | 61.21 | O |
| ATOM | 17940 | N | THR | C | 376 | 20.735 | 43.830 | 59.472 | 1.00 | 69.92 | N |
| ATOM | 17942 | CA | THR | C | 376 | 20.191 | 42.515 | 59.661 | 1.00 | 72.22 | C |
| ATOM | 17944 | CB | THR | C | 376 | 21.334 | 41.602 | 59.808 | 1.00 | 68.57 | C |
| ATOM | 17946 | OG1 | THR | C | 376 | 22.053 | 41.618 | 58.582 | 1.00 | 71.14 | O |
| ATOM | 17948 | CG2 | THR | C | 376 | 20.874 | 40.181 | 59.945 | 1.00 | 74.54 | C |
| ATOM | 17952 | C | THR | C | 376 | 19.394 | 42.475 | 60.931 | 1.00 | 76.89 | C |
| ATOM | 17953 | O | THR | C | 376 | 19.568 | 43.345 | 61.802 | 1.00 | 77.58 | O |
| ATOM | 17954 | N | LEU | C | 377 | 18.515 | 41.483 | 61.041 | 1.00 | 78.66 | N |
| ATOM | 17956 | CA | LEU | C | 377 | 17.779 | 41.340 | 62.271 | 1.00 | 81.96 | C |
| ATOM | 17958 | CB | LEU | C | 377 | 16.384 | 42.007 | 62.166 | 1.00 | 82.78 | C |
| ATOM | 17961 | CG | LEU | C | 377 | 16.197 | 43.562 | 62.077 | 1.00 | 80.18 | C |
| ATOM | 17963 | CD1 | LEU | C | 377 | 14.947 | 43.957 | 62.853 | 1.00 | 78.29 | C |
| ATOM | 17967 | CD2 | LEU | C | 377 | 17.354 | 44.441 | 62.577 | 1.00 | 78.91 | C |
| ATOM | 17971 | C | LEU | C | 377 | 17.724 | 39.892 | 62.815 | 1.00 | 85.19 | C |
| ATOM | 17972 | O | LEU | C | 377 | 17.679 | 38.869 | 62.076 | 1.00 | 84.41 | O |
| ATOM | 17973 | N | ASP | C | 378 | 17.759 | 39.855 | 64.146 | 1.00 | 85.34 | N |
| ATOM | 17975 | CA | ASP | C | 378 | 17.597 | 38.638 | 64.925 | 1.00 | 86.48 | C |
| ATOM | 17977 | CB | ASP | C | 378 | 16.107 | 38.328 | 65.098 | 1.00 | 87.44 | C |
| ATOM | 17980 | CG | ASP | C | 378 | 15.461 | 39.248 | 66.154 | 1.00 | 89.47 | C |
| ATOM | 17981 | OD1 | ASP | C | 378 | 14.462 | 39.975 | 65.855 | 1.00 | 84.97 | O |
| ATOM | 17982 | OD2 | ASP | C | 378 | 15.931 | 39.308 | 67.324 | 1.00 | 86.14 | O |
| ATOM | 17983 | C | ASP | C | 378 | 18.427 | 37.522 | 64.336 | 1.00 | 85.13 | C |
| ATOM | 17984 | O | ASP | C | 378 | 17.942 | 36.486 | 63.887 | 1.00 | 81.67 | O |
| ATOM | 17985 | N | MET | C | 379 | 19.722 | 37.805 | 64.374 | 1.00 | 84.77 | N |
| ATOM | 17987 | CA | MET | C | 379 | 20.750 | 36.965 | 63.819 | 1.00 | 82.56 | C |
| ATOM | 17989 | CB | MET | C | 379 | 22.027 | 37.805 | 63.684 | 1.00 | 81.97 | C |
| ATOM | 17992 | CG | MET | C | 379 | 21.955 | 38.836 | 62.581 | 1.00 | 79.38 | C |
| ATOM | 17995 | SD | MET | C | 379 | 23.553 | 39.475 | 62.065 | 1.00 | 83.45 | S |
| ATOM | 17996 | CE | MET | C | 379 | 23.573 | 41.080 | 62.930 | 1.00 | 84.33 | C |
| ATOM | 18000 | C | MET | C | 379 | 21.010 | 35.728 | 64.668 | 1.00 | 84.75 | C |
| ATOM | 18001 | O | MET | C | 379 | 21.060 | 34.618 | 64.133 | 1.00 | 82.43 | O |
| ATOM | 18002 | N | GLU | C | 380 | 21.163 | 35.920 | 65.986 | 1.00 | 87.40 | N |
| ATOM | 18004 | CA | GLU | C | 380 | 21.555 | 34.840 | 66.914 | 1.00 | 87.60 | C |
| ATOM | 18006 | CB | GLU | C | 380 | 21.977 | 35.423 | 68.276 | 1.00 | 89.46 | C |
| ATOM | 18009 | CG | GLU | C | 380 | 22.952 | 34.592 | 69.137 | 1.00 | 93.46 | C |
| ATOM | 18012 | CD | GLU | C | 380 | 23.920 | 33.680 | 68.372 | 1.00 | 95.12 | C |
| ATOM | 18013 | OE1 | GLU | C | 380 | 25.124 | 34.033 | 68.271 | 1.00 | 90.03 | O |
| ATOM | 18014 | OE2 | GLU | C | 380 | 23.480 | 32.596 | 67.888 | 1.00 | 96.29 | O |
| ATOM | 18015 | C | GLU | C | 380 | 20.459 | 33.763 | 67.031 | 1.00 | 86.12 | C |
| ATOM | 18016 | O | GLU | C | 380 | 20.684 | 32.687 | 67.584 | 1.00 | 86.59 | O |
| ATOM | 18017 | N | ASP | C | 381 | 19.274 | 34.075 | 66.511 | 1.00 | 83.49 | N |
| ATOM | 18019 | CA | ASP | C | 381 | 18.248 | 33.064 | 66.258 | 1.00 | 81.88 | C |
| ATOM | 18021 | CB | ASP | C | 381 | 16.884 | 33.717 | 66.074 | 1.00 | 84.33 | C |
| ATOM | 18024 | CG | ASP | C | 381 | 16.354 | 34.306 | 67.351 | 1.00 | 88.13 | C |
| ATOM | 18025 | OD1 | ASP | C | 381 | 17.009 | 34.123 | 68.400 | 1.00 | 92.81 | O |
| ATOM | 18026 | OD2 | ASP | C | 381 | 15.285 | 34.957 | 67.412 | 1.00 | 96.45 | O |
| ATOM | 18027 | C | ASP | C | 381 | 18.542 | 32.177 | 65.026 | 1.00 | 77.79 | C |
| ATOM | 18028 | O | ASP | C | 381 | 17.711 | 31.347 | 64.663 | 1.00 | 73.78 | O |
| ATOM | 18029 | N | CYS | C | 382 | 19.691 | 32.376 | 64.374 | 1.00 | 76.40 | N |
| ATOM | 18031 | CA | CYS | C | 382 | 20.187 | 31.458 | 63.331 | 1.00 | 74.85 | C |
| ATOM | 18033 | CB | CYS | C | 382 | 20.891 | 32.215 | 62.222 | 1.00 | 75.98 | C |
| ATOM | 18036 | SG | CYS | C | 382 | 19.819 | 33.500 | 61.620 | 1.00 | 81.03 | S |
| ATOM | 18037 | C | CYS | C | 382 | 21.192 | 30.538 | 63.948 | 1.00 | 71.07 | C |
| ATOM | 18038 | O | CYS | C | 382 | 21.755 | 29.673 | 63.284 | 1.00 | 71.77 | O |
| ATOM | 18039 | N | GLY | C | 383 | 21.432 | 30.753 | 65.230 | 1.00 | 70.20 | N |
| ATOM | 18041 | CA | GLY | C | 383 | 22.315 | 29.896 | 65.987 | 1.00 | 71.89 | C |
| ATOM | 18044 | C | GLY | C | 383 | 21.508 | 28.693 | 66.386 | 1.00 | 67.41 | C |
| ATOM | 18045 | O | GLY | C | 383 | 20.307 | 28.772 | 66.344 | 1.00 | 63.32 | O |
| ATOM | 18046 | N | TYR | C | 384 | 22.173 | 27.610 | 66.777 | 1.00 | 70.66 | N |
| ATOM | 18048 | CA | TYR | C | 384 | 21.524 | 26.326 | 67.055 | 1.00 | 70.77 | C |
| ATOM | 18050 | CB | TYR | C | 384 | 22.069 | 25.337 | 66.056 | 1.00 | 73.27 | C |
| ATOM | 18053 | CG | TYR | C | 384 | 21.523 | 23.975 | 66.237 | 1.00 | 78.48 | C |
| ATOM | 18054 | CD1 | TYR | C | 384 | 20.204 | 23.719 | 65.946 | 1.00 | 81.09 | C |
| ATOM | 18056 | CE1 | TYR | C | 384 | 19.676 | 22.472 | 66.103 | 1.00 | 85.92 | C |
| ATOM | 18058 | CZ | TYR | C | 384 | 20.465 | 21.448 | 66.560 | 1.00 | 86.01 | C |
| ATOM | 18059 | OH | TYR | C | 384 | 19.876 | 20.203 | 66.701 | 1.00 | 90.24 | O |
| ATOM | 18061 | CE2 | TYR | C | 384 | 21.805 | 21.676 | 66.865 | 1.00 | 85.31 | C |
| ATOM | 18063 | CD2 | TYR | C | 384 | 22.322 | 22.936 | 66.701 | 1.00 | 81.62 | C |
| ATOM | 18065 | C | TYR | C | 384 | 21.815 | 25.839 | 68.471 | 1.00 | 69.90 | C |
| ATOM | 18066 | O | TYR | C | 384 | 22.877 | 26.160 | 68.995 | 1.00 | 72.05 | O |
| ATOM | 18067 | N | ASN | C | 385 | 20.917 | 25.061 | 69.091 | 1.00 | 70.22 | N |
| ATOM | 18069 | CA | ASN | C | 385 | 21.075 | 24.714 | 70.531 | 1.00 | 71.13 | C |
| ATOM | 18071 | CB | ASN | C | 385 | 20.189 | 25.677 | 71.312 | 1.00 | 69.28 | C |
| ATOM | 18074 | CG | ASN | C | 385 | 20.760 | 27.093 | 71.323 | 1.00 | 71.39 | C |
| ATOM | 18075 | OD1 | ASN | C | 385 | 21.959 | 27.270 | 71.578 | 1.00 | 73.33 | O |
| ATOM | 18076 | ND2 | ASN | C | 385 | 19.922 | 28.103 | 71.047 | 1.00 | 59.22 | N |
| ATOM | 18079 | C | ASN | C | 385 | 20.886 | 23.262 | 71.086 | 1.00 | 73.98 | C |
| ATOM | 18080 | O | ASN | C | 385 | 19.844 | 22.617 | 71.262 | 1.00 | 78.23 | O |
| ATOM | 18081 | OXT | ASN | C | 385 | 21.764 | 22.479 | 71.486 | 1.00 | 70.20 | O |
| ATOM | 18082 | N | GLU | D | 1 | 24.811 | 10.412 | 15.956 | 1.00 | 49.83 | N |
| ATOM | 18084 | CA | GLU | D | 1 | 24.832 | 9.932 | 14.556 | 1.00 | 49.27 | C |
| ATOM | 18086 | CB | GLU | D | 1 | 24.711 | 11.160 | 13.647 | 1.00 | 52.47 | C |
| ATOM | 18089 | CG | GLU | D | 1 | 24.175 | 10.965 | 12.229 | 1.00 | 53.06 | C |
| ATOM | 18092 | CD | GLU | D | 1 | 23.832 | 12.282 | 11.531 | 1.00 | 54.58 | C |
| ATOM | 18093 | OE1 | GLU | D | 1 | 23.120 | 13.161 | 12.125 | 1.00 | 58.12 | O |
| ATOM | 18094 | OE2 | GLU | D | 1 | 24.276 | 12.444 | 10.373 | 1.00 | 51.77 | O |
| ATOM | 18095 | C | GLU | D | 1 | 26.172 | 9.309 | 14.292 | 1.00 | 53.40 | C |
| ATOM | 18096 | O | GLU | D | 1 | 27.170 | 9.803 | 14.821 | 1.00 | 62.53 | O |
| ATOM | 18099 | N | VAL | D | 2 | 26.219 | 8.207 | 13.540 | 1.00 | 50.49 | N |
| ATOM | 18101 | CA | VAL | D | 2 | 27.451 | 7.883 | 12.839 | 1.00 | 47.46 | C |
| ATOM | 18103 | CB | VAL | D | 2 | 27.878 | 6.495 | 13.081 | 1.00 | 48.05 | C |
| ATOM | 18105 | CG1 | VAL | D | 2 | 28.636 | 6.435 | 14.387 | 1.00 | 43.66 | C |
| ATOM | 18109 | CG2 | VAL | D | 2 | 26.682 | 5.606 | 13.046 | 1.00 | 48.85 | C |
| ATOM | 18113 | C | VAL | D | 2 | 27.416 | 8.076 | 11.318 | 1.00 | 46.38 | C |
| ATOM | 18114 | O | VAL | D | 2 | 26.397 | 8.224 | 10.710 | 1.00 | 45.59 | O |
| ATOM | 18115 | N | ASN | D | 3 | 28.589 | 8.071 | 10.719 | 1.00 | 46.83 | N |
| ATOM | 18117 | CA | ASN | D | 3 | 28.728 | 8.117 | 9.292 | 1.00 | 48.82 | C |
| ATOM | 18119 | CB | ASN | D | 3 | 28.844 | 9.528 | 8.743 | 1.00 | 47.44 | C |
| ATOM | 18122 | CG | ASN | D | 3 | 27.547 | 10.251 | 8.771 | 1.00 | 50.24 | C |
| ATOM | 18123 | OD1 | ASN | D | 3 | 27.033 | 10.674 | 7.743 | 1.00 | 45.24 | O |
| ATOM | 18124 | ND2 | ASN | D | 3 | 26.998 | 10.409 | 9.963 | 1.00 | 53.88 | N |
| ATOM | 18127 | C | ASN | D | 3 | 30.011 | 7.423 | 9.016 | 1.00 | 45.35 | C |
| ATOM | 18128 | O | ASN | D | 3 | 30.876 | 8.042 | 8.462 | 1.00 | 56.01 | O |
| ATOM | 18129 | O | LOL | D | 4 | 30.649 | 3.658 | 7.253 | 1.00 | 41.49 | O |
| ATOM | 18130 | C | LOL | D | 4 | 31.616 | 4.701 | 7.302 | 1.00 | 44.31 | C |
| ATOM | 18131 | CA | LOL | D | 4 | 31.464 | 5.422 | 8.643 | 1.00 | 44.47 | C |
| ATOM | 18132 | N | LOL | D | 4 | 30.059 | 5.809 | 8.714 | 1.00 | 40.56 | N |
| ATOM | 18133 | CB | LOL | D | 4 | 31.620 | 4.400 | 9.742 | 1.00 | 39.73 | C |
| ATOM | 18134 | CG | LOL | D | 4 | 31.782 | 5.153 | 11.041 | 1.00 | 42.24 | C |
| ATOM | 18135 | CD2 | LOL | D | 4 | 32.929 | 6.168 | 11.002 | 1.00 | 38.09 | C |
| ATOM | 18136 | CD1 | LOL | D | 4 | 32.012 | 4.153 | 12.156 | 1.00 | 44.46 | C |
| ATOM | 18137 | O | ALQ | D | 5 | 33.539 | 6.138 | 4.922 | 1.00 | 46.58 | O |
| ATOM | 18138 | C | ALQ | D | 5 | 32.895 | 5.448 | 4.118 | 1.00 | 40.59 | C |
| ATOM | 18139 | CA | ALQ | D | 5 | 31.531 | 4.899 | 4.429 | 1.00 | 34.48 | C |
| ATOM | 18140 | CM | ALQ | D | 5 | 31.262 | 5.085 | 5.904 | 1.00 | 25.68 | C |
| ATOM | 18141 | CB | ALQ | D | 5 | 30.525 | 5.761 | 3.689 | 1.00 | 32.81 | C |
| ATOM | 18142 | N | ALA | D | 6 | 33.833 | 4.377 | 4.106 | 1.00 | 47.34 | N |
| ATOM | 18144 | CA | ALA | D | 6 | 35.183 | 4.871 | 3.785 | 1.00 | 46.63 | C |
| ATOM | 18146 | CB | ALA | D | 6 | 35.992 | 3.752 | 3.209 | 1.00 | 48.42 | C |
| ATOM | 18150 | C | ALA | D | 6 | 35.139 | 6.050 | 2.825 | 1.00 | 48.53 | C |
| ATOM | 18151 | O | ALA | D | 6 | 34.387 | 6.073 | 1.850 | 1.00 | 41.51 | O |
| ATOM | 18154 | N | GLU | D | 7 | 35.936 | 7.053 | 3.153 | 1.00 | 57.71 | N |
| ATOM | 18156 | CA | GLU | D | 7 | 36.170 | 8.193 | 2.282 | 1.00 | 61.39 | C |
| ATOM | 18158 | CB | GLU | D | 7 | 37.103 | 9.150 | 2.997 | 1.00 | 63.36 | C |
| ATOM | 18161 | CG | GLU | D | 7 | 36.554 | 10.567 | 3.088 | 1.00 | 69.77 | C |
| ATOM | 18164 | CD | GLU | D | 7 | 37.556 | 11.591 | 3.638 | 1.00 | 72.88 | C |
| ATOM | 18165 | OE1 | GLU | D | 7 | 37.130 | 12.437 | 4.470 | 1.00 | 71.19 | O |
| ATOM | 18166 | OE2 | GLU | D | 7 | 38.753 | 11.548 | 3.245 | 1.00 | 71.30 | O |
| ATOM | 18167 | C | GLU | D | 7 | 36.746 | 7.878 | 0.873 | 1.00 | 63.19 | C |
| ATOM | 18168 | O | GLU | D | 7 | 36.460 | 8.610 | −0.067 | 1.00 | 59.17 | O |
| ATOM | 18169 | N | PHE | D | 8 | 37.554 | 6.833 | 0.708 | 1.00 | 64.51 | N |
| ATOM | 18171 | CA | PHE | D | 8 | 38.046 | 6.472 | −0.636 | 1.00 | 66.42 | C |
| ATOM | 18173 | CB | PHE | D | 8 | 37.450 | 5.145 | −1.093 | 1.00 | 61.61 | C |
| ATOM | 18176 | CG | PHE | D | 8 | 38.061 | 4.586 | −2.379 | 1.00 | 58.86 | C |
| ATOM | 18177 | CD1 | PHE | D | 8 | 37.441 | 4.769 | −3.602 | 1.00 | 59.33 | C |
| ATOM | 18179 | CE1 | PHE | D | 8 | 37.986 | 4.253 | −4.767 | 1.00 | 58.96 | C |
| ATOM | 18181 | CZ | PHE | D | 8 | 39.174 | 3.536 | −4.731 | 1.00 | 60.39 | C |
| ATOM | 18183 | CE2 | PHE | D | 8 | 39.800 | 3.345 | −3.536 | 1.00 | 58.60 | C |
| ATOM | 18185 | CD2 | PHE | D | 8 | 39.239 | 3.869 | −2.355 | 1.00 | 59.06 | C |
| ATOM | 18187 | C | PHE | D | 8 | 37.706 | 7.526 | −1.692 | 1.00 | 70.25 | C |
| ATOM | 18188 | O | PHE | D | 8 | 36.951 | 7.302 | −2.641 | 1.00 | 73.78 | O |
| ATOM | 18189 | OXT | PHE | D | 8 | 38.159 | 8.667 | −1.670 | 1.00 | 76.04 | O |
| ATOM | 18190 | N | GLU | E | 1 | 3.521 | 56.829 | 27.990 | 1.00 | 51.90 | N |
| ATOM | 18192 | CA | GLU | E | 1 | 4.813 | 56.347 | 27.405 | 1.00 | 53.95 | C |
| ATOM | 18194 | CB | GLU | E | 1 | 4.748 | 54.908 | 26.863 | 1.00 | 52.89 | C |
| ATOM | 18197 | CG | GLU | E | 1 | 6.020 | 54.441 | 26.144 | 1.00 | 54.53 | C |
| ATOM | 18200 | CD | GLU | E | 1 | 5.940 | 53.023 | 25.544 | 1.00 | 61.12 | C |
| ATOM | 18201 | OE1 | GLU | E | 1 | 4.802 | 52.481 | 25.400 | 1.00 | 68.56 | O |
| ATOM | 18202 | OE2 | GLU | E | 1 | 7.009 | 52.427 | 25.193 | 1.00 | 55.04 | O |
| ATOM | 18203 | C | GLU | E | 1 | 5.206 | 57.280 | 26.290 | 1.00 | 56.24 | C |
| ATOM | 18204 | O | GLU | E | 1 | 4.726 | 57.179 | 25.177 | 1.00 | 58.77 | O |
| ATOM | 18207 | N | VAL | E | 2 | 6.069 | 58.225 | 26.625 | 1.00 | 60.38 | N |
| ATOM | 18209 | CA | VAL | E | 2 | 6.748 | 59.058 | 25.641 | 1.00 | 55.03 | C |
| ATOM | 18211 | CB | VAL | E | 2 | 7.513 | 60.074 | 26.359 | 1.00 | 53.73 | C |
| ATOM | 18213 | CG1 | VAL | E | 2 | 6.581 | 60.848 | 27.263 | 1.00 | 55.96 | C |
| ATOM | 18217 | CG2 | VAL | E | 2 | 8.615 | 59.394 | 27.171 | 1.00 | 56.70 | C |
| ATOM | 18221 | C | VAL | E | 2 | 7.758 | 58.285 | 24.804 | 1.00 | 54.06 | C |
| ATOM | 18222 | O | VAL | E | 2 | 8.268 | 57.267 | 25.214 | 1.00 | 51.79 | O |
| ATOM | 18223 | N | ASN | E | 3 | 8.053 | 58.785 | 23.618 | 1.00 | 51.49 | N |
| ATOM | 18225 | CA | ASN | E | 3 | 9.062 | 58.167 | 22.787 | 1.00 | 49.08 | C |
| ATOM | 18227 | CB | ASN | E | 3 | 8.502 | 56.972 | 22.011 | 1.00 | 52.23 | C |
| ATOM | 18230 | CG | ASN | E | 3 | 8.790 | 55.632 | 22.678 | 1.00 | 58.18 | C |
| ATOM | 18231 | OD1 | ASN | E | 3 | 9.955 | 55.264 | 22.882 | 1.00 | 65.86 | O |
| ATOM | 18232 | ND2 | ASN | E | 3 | 7.724 | 54.885 | 23.009 | 1.00 | 59.14 | N |
| ATOM | 18235 | C | ASN | E | 3 | 9.599 | 59.235 | 21.836 | 1.00 | 48.29 | C |
| ATOM | 18236 | O | ASN | E | 3 | 9.547 | 59.112 | 20.605 | 1.00 | 43.64 | O |
| ATOM | 18237 | O | LOL | E | 4 | 12.598 | 62.494 | 21.688 | 1.00 | 44.76 | O |
| ATOM | 18238 | C | LOL | E | 4 | 11.793 | 61.498 | 21.082 | 1.00 | 36.75 | C |
| ATOM | 18239 | CA | LOL | E | 4 | 10.394 | 61.683 | 21.640 | 1.00 | 39.29 | C |
| ATOM | 18240 | N | LOL | E | 4 | 10.081 | 60.567 | 22.545 | 1.00 | 35.78 | N |
| ATOM | 18241 | CB | LOL | E | 4 | 10.243 | 62.899 | 22.530 | 1.00 | 30.76 | C |
| ATOM | 18242 | CG | LOL | E | 4 | 8.730 | 62.693 | 22.652 | 1.00 | 40.35 | C |
| ATOM | 18243 | CD2 | LOL | E | 4 | 8.185 | 62.234 | 21.296 | 1.00 | 31.81 | C |
| ATOM | 18244 | CD1 | LOL | E | 4 | 8.003 | 63.914 | 23.151 | 1.00 | 38.31 | C |
| ATOM | 18245 | O | ALQ | E | 5 | 12.574 | 60.630 | 18.017 | 1.00 | 56.62 | O |
| ATOM | 18246 | C | ALQ | E | 5 | 12.792 | 61.258 | 19.046 | 1.00 | 45.00 | C |
| ATOM | 18247 | CA | ALQ | E | 5 | 13.506 | 60.509 | 20.107 | 1.00 | 39.17 | C |
| ATOM | 18248 | CM | ALQ | E | 5 | 12.383 | 59.943 | 20.954 | 1.00 | 33.18 | C |
| ATOM | 18249 | CB | ALQ | E | 5 | 14.198 | 59.370 | 19.382 | 1.00 | 38.30 | C |
| ATOM | 18250 | N | ALA | E | 6 | 13.763 | 61.587 | 17.911 | 1.00 | 53.06 | N |
| ATOM | 18252 | CA | ALA | E | 6 | 13.416 | 62.316 | 16.681 | 1.00 | 48.77 | C |
| ATOM | 18254 | CB | ALA | E | 6 | 14.238 | 63.578 | 16.590 | 1.00 | 46.81 | C |
| ATOM | 18258 | C | ALA | E | 6 | 13.739 | 61.376 | 15.550 | 1.00 | 56.62 | C |
| ATOM | 18259 | O | ALA | E | 6 | 14.884 | 61.001 | 15.355 | 1.00 | 60.60 | O |
| ATOM | 18262 | N | GLU | E | 7 | 12.718 | 60.941 | 14.836 | 1.00 | 66.83 | N |
| ATOM | 18264 | CA | GLU | E | 7 | 12.894 | 60.209 | 13.578 | 1.00 | 70.79 | C |
| ATOM | 18266 | CB | GLU | E | 7 | 11.658 | 60.366 | 12.670 | 1.00 | 71.58 | C |
| ATOM | 18269 | CG | GLU | E | 7 | 11.195 | 58.999 | 12.147 | 1.00 | 74.78 | C |
| ATOM | 18272 | CD | GLU | E | 7 | 9.767 | 58.949 | 11.583 | 1.00 | 77.65 | C |
| ATOM | 18273 | OE1 | GLU | E | 7 | 9.502 | 59.494 | 10.475 | 1.00 | 71.21 | O |
| ATOM | 18274 | OE2 | GLU | E | 7 | 8.907 | 58.325 | 12.252 | 1.00 | 77.08 | O |
| ATOM | 18275 | C | GLU | E | 7 | 14.184 | 60.468 | 12.764 | 1.00 | 72.67 | C |
| ATOM | 18276 | O | GLU | E | 7 | 15.094 | 59.622 | 12.765 | 1.00 | 77.73 | O |
| ATOM | 18277 | N | PHE | E | 8 | 14.297 | 61.598 | 12.076 | 1.00 | 75.16 | N |
| ATOM | 18279 | CA | PHE | E | 8 | 15.396 | 61.740 | 11.113 | 1.00 | 80.96 | C |
| ATOM | 18281 | CB | PHE | E | 8 | 16.737 | 61.952 | 11.801 | 1.00 | 82.87 | C |
| ATOM | 18284 | CG | PHE | E | 8 | 17.803 | 62.573 | 10.917 | 1.00 | 81.73 | C |
| ATOM | 18285 | CD1 | PHE | E | 8 | 18.769 | 61.793 | 10.279 | 1.00 | 83.54 | C |
| ATOM | 18287 | CE1 | PHE | E | 8 | 19.751 | 62.381 | 9.479 | 1.00 | 81.99 | C |
| ATOM | 18289 | CZ | PHE | E | 8 | 19.763 | 63.746 | 9.316 | 1.00 | 83.35 | C |
| ATOM | 18291 | CE2 | PHE | E | 8 | 18.811 | 64.520 | 9.944 | 1.00 | 82.55 | C |
| ATOM | 18293 | CD2 | PHE | E | 8 | 17.841 | 63.934 | 10.739 | 1.00 | 82.70 | C |
| ATOM | 18295 | C | PHE | E | 8 | 15.507 | 60.437 | 10.291 | 1.00 | 85.47 | C |
| ATOM | 18296 | O | PHE | E | 8 | 16.608 | 59.978 | 9.946 | 1.00 | 83.09 | O |
| ATOM | 18297 | OXT | PHE | E | 8 | 14.521 | 59.767 | 9.937 | 1.00 | 89.34 | O |
| ATOM | 18298 | N | GLU | F | 1 | 40.126 | 14.124 | 47.515 | 1.00 | 62.65 | N |
| ATOM | 18300 | CA | GLU | F | 1 | 39.361 | 14.895 | 48.535 | 1.00 | 57.33 | C |
| ATOM | 18302 | CB | GLU | F | 1 | 37.879 | 14.527 | 48.449 | 1.00 | 55.81 | C |
| ATOM | 18305 | CG | GLU | F | 1 | 36.898 | 15.591 | 48.922 | 1.00 | 53.48 | C |
| ATOM | 18308 | CD | GLU | F | 1 | 35.432 | 15.241 | 48.626 | 1.00 | 57.95 | C |
| ATOM | 18309 | OE1 | GLU | F | 1 | 35.172 | 14.718 | 47.492 | 1.00 | 57.16 | O |
| ATOM | 18310 | OE2 | GLU | F | 1 | 34.540 | 15.499 | 49.515 | 1.00 | 46.53 | O |
| ATOM | 18311 | C | GLU | F | 1 | 39.838 | 14.584 | 49.925 | 1.00 | 56.46 | C |
| ATOM | 18312 | O | GLU | F | 1 | 39.954 | 13.421 | 50.262 | 1.00 | 59.04 | O |
| ATOM | 18315 | N | VAL | F | 2 | 40.171 | 15.626 | 50.694 | 1.00 | 52.85 | N |
| ATOM | 18317 | CA | VAL | F | 2 | 40.258 | 15.529 | 52.154 | 1.00 | 48.66 | C |
| ATOM | 18319 | CB | VAL | F | 2 | 41.331 | 16.368 | 52.682 | 1.00 | 45.45 | C |
| ATOM | 18321 | CG1 | VAL | F | 2 | 42.586 | 15.583 | 52.593 | 1.00 | 48.71 | C |
| ATOM | 18325 | CG2 | VAL | F | 2 | 41.395 | 17.658 | 51.885 | 1.00 | 48.21 | C |
| ATOM | 18329 | C | VAL | F | 2 | 39.011 | 15.976 | 52.885 | 1.00 | 45.82 | C |
| ATOM | 18330 | O | VAL | F | 2 | 38.300 | 16.826 | 52.430 | 1.00 | 52.38 | O |
| ATOM | 18331 | N | ASN | F | 3 | 38.762 | 15.399 | 54.047 | 1.00 | 47.13 | N |
| ATOM | 18333 | CA | ASN | F | 3 | 37.566 | 15.702 | 54.814 | 1.00 | 48.46 | C |
| ATOM | 18335 | CB | ASN | F | 3 | 36.425 | 14.705 | 54.512 | 1.00 | 46.45 | C |
| ATOM | 18338 | CG | ASN | F | 3 | 35.499 | 15.178 | 53.371 | 1.00 | 48.95 | C |
| ATOM | 18339 | OD1 | ASN | F | 3 | 34.662 | 16.042 | 53.600 | 1.00 | 56.94 | O |
| ATOM | 18340 | ND2 | ASN | F | 3 | 35.637 | 14.599 | 52.147 | 1.00 | 41.31 | N |
| ATOM | 18343 | C | ASN | F | 3 | 37.938 | 15.676 | 56.289 | 1.00 | 48.81 | C |
| ATOM | 18344 | O | ASN | F | 3 | 37.884 | 14.650 | 56.945 | 1.00 | 46.42 | O |
| ATOM | 18345 | O | LOL | F | 4 | 39.109 | 18.145 | 59.529 | 1.00 | 30.33 | O |
| ATOM | 18346 | C | LOL | F | 4 | 39.139 | 16.727 | 59.642 | 1.00 | 37.57 | C |
| ATOM | 18347 | CA | LOL | F | 4 | 39.449 | 15.978 | 58.340 | 1.00 | 34.22 | C |
| ATOM | 18348 | N | LOL | F | 4 | 38.607 | 16.653 | 57.414 | 1.00 | 33.81 | N |
| ATOM | 18349 | CB | LOL | F | 4 | 40.854 | 16.178 | 57.816 | 1.00 | 29.74 | C |
| ATOM | 18350 | CG | LOL | F | 4 | 41.230 | 14.854 | 57.136 | 1.00 | 42.88 | C |
| ATOM | 18351 | CD2 | LOL | F | 4 | 40.898 | 13.685 | 58.067 | 1.00 | 44.40 | C |
| ATOM | 18352 | CD1 | LOL | F | 4 | 42.690 | 14.689 | 56.716 | 1.00 | 33.62 | C |
| ATOM | 18353 | O | ALQ | F | 5 | 37.076 | 16.340 | 61.729 | 1.00 | 51.12 | O |
| ATOM | 18354 | C | ALQ | F | 5 | 37.482 | 17.448 | 61.387 | 1.00 | 44.19 | C |
| ATOM | 18355 | CA | ALQ | F | 5 | 37.023 | 17.885 | 60.039 | 1.00 | 45.38 | C |
| ATOM | 18356 | CM | ALQ | F | 5 | 37.387 | 16.833 | 58.971 | 1.00 | 30.19 | C |
| ATOM | 18357 | CB | ALQ | F | 5 | 35.522 | 17.952 | 60.244 | 1.00 | 38.38 | C |
| ATOM | 18358 | N | ALA | F | 6 | 37.907 | 15.961 | 62.627 | 1.00 | 55.63 | N |
| ATOM | 18360 | CA | ALA | F | 6 | 37.280 | 15.766 | 63.961 | 1.00 | 48.80 | C |
| ATOM | 18362 | CB | ALA | F | 6 | 37.591 | 16.875 | 64.882 | 1.00 | 43.42 | C |
| ATOM | 18366 | C | ALA | F | 6 | 35.791 | 15.554 | 63.915 | 1.00 | 54.73 | C |
| ATOM | 18367 | O | ALA | F | 6 | 35.009 | 16.459 | 64.126 | 1.00 | 60.39 | O |
| ATOM | 18370 | N | GLU | F | 7 | 35.453 | 14.325 | 63.591 | 1.00 | 61.57 | N |
| ATOM | 18372 | CA | GLU | F | 7 | 34.181 | 13.687 | 63.917 | 1.00 | 67.60 | C |
| ATOM | 18374 | CB | GLU | F | 7 | 34.182 | 12.237 | 63.388 | 1.00 | 70.70 | C |
| ATOM | 18377 | CG | GLU | F | 7 | 32.813 | 11.775 | 62.910 | 1.00 | 73.43 | C |
| ATOM | 18380 | CD | GLU | F | 7 | 32.354 | 10.447 | 63.500 | 1.00 | 77.46 | C |
| ATOM | 18381 | OE1 | GLU | F | 7 | 32.607 | 10.173 | 64.697 | 1.00 | 77.46 | O |
| ATOM | 18382 | OE2 | GLU | F | 7 | 31.714 | 9.677 | 62.756 | 1.00 | 78.85 | O |
| ATOM | 18383 | C | GLU | F | 7 | 33.791 | 13.545 | 65.424 | 1.00 | 65.86 | C |
| ATOM | 18384 | O | GLU | F | 7 | 33.074 | 12.629 | 65.731 | 1.00 | 68.96 | O |
| ATOM | 18385 | N | PHE | F | 8 | 34.287 | 14.366 | 66.347 | 1.00 | 67.95 | N |
| ATOM | 18387 | CA | PHE | F | 8 | 33.718 | 14.541 | 67.716 | 1.00 | 67.25 | C |
| ATOM | 18389 | CB | PHE | F | 8 | 34.303 | 15.837 | 68.301 | 1.00 | 68.83 | C |
| ATOM | 18392 | CG | PHE | F | 8 | 33.612 | 16.393 | 69.544 | 1.00 | 64.14 | C |
| ATOM | 18393 | CD1 | PHE | F | 8 | 32.493 | 17.192 | 69.441 | 1.00 | 65.62 | C |
| ATOM | 18395 | CE1 | PHE | F | 8 | 31.873 | 17.724 | 70.583 | 1.00 | 64.20 | C |
| ATOM | 18397 | CZ | PHE | F | 8 | 32.376 | 17.472 | 71.840 | 1.00 | 63.31 | C |
| ATOM | 18399 | CE2 | PHE | F | 8 | 33.496 | 16.688 | 71.971 | 1.00 | 65.43 | C |
| ATOM | 18401 | CD2 | PHE | F | 8 | 34.123 | 16.150 | 70.817 | 1.00 | 66.04 | C |
| ATOM | 18403 | C | PHE | F | 8 | 32.209 | 14.659 | 67.761 | 1.00 | 71.68 | C |
| ATOM | 18404 | O | PHE | F | 8 | 31.610 | 14.612 | 68.854 | 1.00 | 73.64 | O |
| ATOM | 18405 | OXT | PHE | F | 8 | 31.569 | 14.808 | 66.717 | 1.00 | 76.55 | O |
| ATOM | 18406 | O | HOH | W | 1 | 39.700 | 13.653 | 43.690 | 1.00 | 64.49 | O |
| ATOM | 18409 | O | HOH | W | 2 | 17.999 | −0.835 | 18.526 | 1.00 | 57.08 | O |
| ATOM | 18412 | O | HOH | W | 3 | −2.213 | 39.679 | 44.264 | 1.00 | 47.81 | O |
| ATOM | 18415 | O | HOH | W | 4 | 44.390 | 21.700 | 54.614 | 1.00 | 36.23 | O |
| ATOM | 18418 | O | HOH | W | 5 | 44.036 | 10.520 | 13.671 | 1.00 | 20.25 | O |
| ATOM | 18421 | O | HOH | W | 6 | 21.081 | 11.327 | 18.984 | 1.00 | 79.68 | O |
| ATOM | 18424 | O | HOH | W | 7 | 12.954 | 48.886 | 45.723 | 1.00 | 47.92 | O |
| ATOM | 18427 | O | HOH | W | 8 | 8.570 | 89.322 | 9.495 | 1.00 | 42.50 | O |
| ATOM | 18430 | O | HOH | W | 9 | 25.965 | −8.446 | −10.244 | 1.00 | 58.04 | O |
| ATOM | 18433 | O | HOH | W | 10 | 11.743 | 63.828 | 30.252 | 1.00 | 60.50 | O |
| ATOM | 18436 | O | HOH | W | 11 | −0.897 | 57.335 | 42.929 | 1.00 | 57.91 | O |
| ATOM | 18439 | O | HOH | W | 12 | 52.750 | 29.899 | 42.854 | 1.00 | 71.96 | O |
| ATOM | 18442 | O | HOH | W | 13 | 31.267 | 56.125 | 22.788 | 1.00 | 67.37 | O |
| ATOM | 18445 | O | HOH | W | 14 | −0.501 | 17.272 | 5.350 | 1.00 | 49.57 | O |
| ATOM | 18448 | O | HOH | W | 15 | 19.139 | 31.098 | 19.928 | 1.00 | 53.73 | O |
| ATOM | 18451 | O | HOH | W | 16 | 29.653 | 40.187 | 36.475 | 1.00 | 51.39 | O |
| ATOM | 18454 | O | HOH | W | 17 | 1.702 | 54.764 | 31.203 | 1.00 | 59.48 | O |
| ATOM | 18457 | O | HOH | W | 18 | 17.103 | −19.576 | 2.357 | 1.00 | 54.90 | O |
| ATOM | 18460 | O | HOH | W | 19 | 10.345 | 16.962 | 33.276 | 1.00 | 66.13 | O |
| ATOM | 18463 | O | HOH | W | 20 | 9.735 | 17.583 | −5.490 | 1.00 | 62.55 | O |
| ATOM | 18466 | O | HOH | W | 21 | 16.189 | 57.395 | 15.723 | 1.00 | 60.50 | O |
| ATOM | 18469 | O | HOH | W | 22 | 17.845 | 79.168 | 18.933 | 1.00 | 43.39 | O |
| ATOM | 18472 | O | HOH | W | 23 | 26.061 | −0.244 | 12.926 | 1.00 | 35.89 | O |
| ATOM | 18475 | O | HOH | W | 24 | 21.349 | −5.739 | −14.919 | 1.00 | 60.98 | O |
| ATOM | 18478 | O | HOH | W | 25 | −4.354 | 53.549 | 53.329 | 1.00 | 77.11 | O |
| ATOM | 18481 | O | HOH | W | 26 | 46.391 | 31.873 | 42.449 | 1.00 | 46.27 | O |
| ATOM | 18484 | O | HOH | W | 27 | 5.794 | 14.925 | 30.466 | 1.00 | 56.03 | O |
| ATOM | 18487 | O | HOH | W | 28 | 18.066 | 33.687 | 53.506 | 1.00 | 60.88 | O |
| ATOM | 18490 | O | HOH | W | 29 | 34.788 | 61.731 | 39.462 | 1.00 | 47.79 | O |
| ATOM | 18493 | O | HOH | W | 30 | 21.025 | 22.947 | 1.341 | 1.00 | 59.51 | O |
| ATOM | 18496 | O | HOH | W | 31 | 16.949 | 23.886 | 38.354 | 1.00 | 45.81 | O |
| ATOM | 18499 | O | HOH | W | 32 | 39.142 | 5.166 | 3.509 | 1.00 | 66.57 | O |
| ATOM | 18502 | O | HOH | W | 33 | 60.666 | 20.142 | 53.793 | 1.00 | 44.92 | O |
| ATOM | 18505 | O | HOH | W | 34 | 42.265 | 8.229 | −0.588 | 1.00 | 60.46 | O |
| ATOM | 18508 | O | HOH | W | 35 | 32.860 | 14.034 | 60.875 | 1.00 | 60.98 | O |
| ATOM | 18511 | O | HOH | W | 36 | 32.941 | 17.022 | 57.493 | 1.00 | 38.19 | O |
| ATOM | 18514 | O | HOH | W | 37 | 29.695 | 61.886 | 47.641 | 1.00 | 59.00 | O |
| ATOM | 18517 | O | HOH | W | 38 | 33.674 | −12.279 | −2.746 | 1.00 | 45.53 | O |
| ATOM | 18520 | O | HOH | W | 39 | 41.413 | −14.843 | 19.171 | 1.00 | 65.48 | O |
| ATOM | 18523 | O | HOH | W | 40 | 24.064 | 6.895 | −21.588 | 1.00 | 70.17 | O |
| ATOM | 18526 | O | HOH | W | 41 | 23.715 | 82.758 | 18.726 | 1.00 | 35.41 | O |
| ATOM | 18529 | O | HOH | W | 42 | 47.525 | −13.020 | 29.129 | 1.00 | 52.35 | O |
| ATOM | 18532 | O | HOH | W | 43 | 36.969 | −15.675 | −0.741 | 1.00 | 53.72 | O |
| ATOM | 18535 | O | HOH | W | 44 | 27.524 | 78.411 | 40.703 | 1.00 | 48.20 | O |
| ATOM | 18538 | O | HOH | W | 45 | 31.107 | 72.075 | 37.162 | 1.00 | 47.99 | O |
| ATOM | 18541 | O | HOH | W | 46 | 21.564 | 34.567 | 37.693 | 1.00 | 46.25 | O |
| ATOM | 18544 | O | HOH | W | 47 | 21.067 | 71.763 | 21.267 | 1.00 | 39.24 | O |
| ATOM | 18547 | O | HOH | W | 48 | 10.404 | 9.098 | 24.542 | 1.00 | 57.88 | O |
| ATOM | 18550 | O | HOH | W | 49 | 5.396 | 65.013 | 32.485 | 1.00 | 60.14 | O |
| ATOM | 18553 | O | HOH | W | 50 | 27.493 | 74.629 | 18.467 | 1.00 | 55.45 | O |
| ATOM | 18556 | O | HOH | W | 51 | 13.872 | 41.111 | 47.061 | 1.00 | 42.07 | O |
| ATOM | 18559 | O | HOH | W | 52 | 9.887 | 61.022 | 39.473 | 1.00 | 62.04 | O |
| ATOM | 18562 | O | HOH | W | 53 | 55.509 | −10.069 | 15.668 | 1.00 | 47.86 | O |
| ATOM | 18565 | O | HOH | W | 54 | 37.626 | 42.674 | 24.706 | 1.00 | 67.33 | O |
| ATOM | 18568 | O | HOH | W | 55 | 55.291 | 39.593 | 51.342 | 1.00 | 47.73 | O |
| ATOM | 18571 | O | HOH | W | 56 | 43.917 | 17.193 | 24.624 | 1.00 | 60.60 | O |
| ATOM | 18574 | O | HOH | W | 57 | 41.122 | 31.365 | 43.632 | 1.00 | 77.01 | O |
| ATOM | 18577 | O | HOH | W | 58 | 13.079 | 35.338 | 39.068 | 1.00 | 88.26 | O |
| ATOM | 18580 | O | HOH | W | 59 | 27.493 | −10.186 | 19.656 | 1.00 | 35.50 | O |
| ATOM | 18583 | O | HOH | W | 60 | 23.506 | 41.302 | 45.173 | 1.00 | 42.69 | O |
| ATOM | 18586 | O | HOH | W | 61 | −0.922 | 57.734 | 39.341 | 1.00 | 85.08 | O |
| ATOM | 18589 | O | HOH | W | 62 | 9.888 | 47.144 | 47.199 | 1.00 | 45.05 | O |
| ATOM | 18592 | O | HOH | W | 63 | 4.711 | 19.735 | 7.209 | 1.00 | 60.99 | O |
| ATOM | 18595 | O | HOH | W | 64 | 20.357 | 53.470 | 8.144 | 1.00 | 51.38 | O |
| ATOM | 18598 | O | HOH | W | 65 | 30.784 | 37.575 | 33.529 | 1.00 | 37.97 | O |
| ATOM | 18601 | O | HOH | W | 66 | 33.822 | −13.870 | 25.347 | 1.00 | 62.92 | O |
| ATOM | 18604 | O | HOH | W | 67 | 31.877 | −4.483 | −12.266 | 1.00 | 54.62 | O |
| ATOM | 18607 | O | HOH | W | 68 | 32.094 | 8.727 | 34.971 | 1.00 | 57.46 | O |
| ATOM | 18610 | O | HOH | W | 69 | 22.667 | 29.780 | 32.323 | 1.00 | 51.06 | O |
| ATOM | 18613 | O | HOH | W | 70 | 62.327 | 6.382 | 57.793 | 1.00 | 65.33 | O |
| ATOM | 18616 | O | HOH | W | 71 | 36.357 | 61.805 | 15.075 | 1.00 | 68.60 | O |
| ATOM | 18619 | O | HOH | W | 72 | 10.784 | 50.207 | 46.403 | 1.00 | 57.99 | O |
| ATOM | 18622 | O | HOH | W | 73 | 34.260 | 6.846 | −14.935 | 1.00 | 52.03 | O |
| ATOM | 18625 | O | HOH | W | 74 | 9.575 | −9.503 | 1.975 | 1.00 | 42.15 | O |
| ATOM | 18628 | O | HOH | W | 75 | 20.501 | 19.388 | 34.503 | 1.00 | 63.39 | O |
| ATOM | 18631 | O | HOH | W | 76 | −5.681 | 42.903 | 44.382 | 1.00 | 47.81 | O |
| ATOM | 18634 | O | HOH | W | 77 | 25.415 | −23.286 | 11.860 | 1.00 | 36.69 | O |
| ATOM | 18637 | O | HOH | W | 78 | 7.045 | 50.529 | 30.338 | 1.00 | 69.50 | O |
| ATOM | 18640 | O | HOH | W | 79 | −5.772 | 89.065 | 18.733 | 1.00 | 61.42 | O |
| ATOM | 18643 | O | HOH | W | 80 | 14.414 | 64.947 | 12.207 | 1.00 | 64.73 | O |
| ATOM | 18646 | O | HOH | W | 81 | 31.802 | 2.198 | −9.686 | 1.00 | 55.42 | O |
| ATOM | 18649 | O | HOH | W | 82 | 10.420 | 30.167 | 51.382 | 1.00 | 50.99 | O |
| ATOM | 18652 | O | HOH | W | 83 | 15.150 | −3.137 | −23.038 | 1.00 | 52.37 | O |
| ATOM | 18655 | O | HOH | W | 84 | 48.107 | 19.301 | 35.815 | 1.00 | 82.63 | O |
| ATOM | 18658 | O | HOH | W | 85 | −1.331 | 82.678 | 16.423 | 1.00 | 57.12 | O |
| ATOM | 18661 | O | HOH | W | 86 | 48.215 | 43.713 | 47.628 | 1.00 | 62.68 | O |
| ATOM | 18664 | O | HOH | W | 87 | 13.293 | 76.226 | 35.713 | 1.00 | 60.16 | O |
| ATOM | 18667 | O | HOH | W | 88 | 42.110 | −10.362 | 7.097 | 1.00 | 50.69 | O |
| ATOM | 18670 | O | HOH | W | 89 | 27.827 | −20.605 | 8.513 | 1.00 | 49.19 | O |
| ATOM | 18673 | O | HOH | W | 90 | 55.096 | 10.432 | 53.072 | 1.00 | 53.87 | O |
| ATOM | 18676 | O | HOH | W | 91 | 34.400 | 69.845 | 30.780 | 1.00 | 51.06 | O |
| ATOM | 18679 | O | HOH | W | 92 | 25.651 | 45.557 | 41.010 | 1.00 | 50.02 | O |
| ATOM | 18682 | O | HOH | W | 93 | −4.352 | 46.330 | 53.290 | 1.00 | 47.26 | O |
| ATOM | 18685 | O | HOH | W | 94 | 21.498 | 87.119 | 29.374 | 1.00 | 64.19 | O |
| ATOM | 18688 | O | HOH | W | 95 | 15.571 | −1.087 | −19.807 | 1.00 | 72.71 | O |
| ATOM | 18691 | O | HOH | W | 96 | 38.100 | 1.417 | 58.870 | 1.00 | 53.38 | O |
| ATOM | 18694 | O | HOH | W | 97 | 23.613 | 3.572 | 11.202 | 1.00 | 47.48 | O |
| ATOM | 18697 | O | HOH | W | 98 | 10.095 | 50.879 | 42.655 | 1.00 | 63.46 | O |
| ATOM | 18700 | O | HOH | W | 99 | 15.664 | −12.915 | 22.110 | 1.00 | 56.85 | O |
| ATOM | 18703 | O | HOH | W | 100 | 16.817 | 73.724 | 20.533 | 1.00 | 40.32 | O |
| ATOM | 18706 | O | HOH | W | 101 | −2.361 | 88.393 | 14.727 | 1.00 | 69.47 | O |
| ATOM | 18709 | O | HOH | W | 102 | 46.723 | −8.667 | 16.202 | 1.00 | 61.97 | O |
| ATOM | 18712 | O | HOH | W | 103 | 41.497 | −4.936 | 5.247 | 1.00 | 35.19 | O |
| ATOM | 18715 | O | HOH | W | 104 | 22.334 | −10.122 | −11.332 | 1.00 | 39.62 | O |
| ATOM | 18718 | O | HOH | W | 105 | 27.866 | 33.167 | 19.639 | 1.00 | 47.41 | O |
| ATOM | 18721 | O | HOH | W | 106 | 51.713 | 4.314 | 11.942 | 1.00 | 66.19 | O |
| ATOM | 18724 | O | HOH | W | 107 | −0.992 | −3.672 | 8.624 | 1.00 | 33.89 | O |
| ATOM | 18727 | O | HOH | W | 108 | 62.335 | 12.316 | 58.893 | 1.00 | 41.09 | O |
| ATOM | 18730 | O | HOH | W | 109 | 33.491 | −5.887 | −10.390 | 1.00 | 41.57 | O |
| ATOM | 18733 | O | HOH | W | 110 | −5.756 | 74.126 | 24.837 | 1.00 | 47.70 | O |
| ATOM | 18736 | O | HOH | W | 111 | 46.526 | 15.070 | 33.144 | 1.00 | 64.45 | O |
| ATOM | 18739 | O | HOH | W | 112 | 18.516 | 31.491 | 45.131 | 1.00 | 66.88 | O |
| ATOM | 18742 | O | HOH | W | 113 | 7.469 | 52.148 | 17.072 | 1.00 | 52.60 | O |
| ATOM | 18745 | O | HOH | W | 114 | 9.252 | 52.803 | 15.021 | 1.00 | 51.30 | O |
| ATOM | 18748 | O | HOH | W | 115 | 36.282 | 18.066 | 45.395 | 1.00 | 47.63 | O |
| ATOM | 18751 | O | HOH | W | 116 | 17.694 | −5.596 | 0.263 | 1.00 | 56.44 | O |
| ATOM | 18754 | O | HOH | W | 117 | 14.604 | −3.959 | 27.588 | 1.00 | 52.41 | O |
| ATOM | 18757 | O | HOH | W | 118 | 56.706 | 25.271 | 55.385 | 1.00 | 50.39 | O |
| ATOM | 18760 | O | HOH | W | 119 | 6.543 | −7.728 | 10.215 | 1.00 | 54.60 | O |
| ATOM | 18763 | O | HOH | W | 120 | 21.354 | −20.233 | 10.919 | 1.00 | 52.16 | O |
| ATOM | 18766 | O | HOH | W | 121 | −1.214 | 7.759 | 12.883 | 1.00 | 46.46 | O |
| ATOM | 18769 | O | HOH | W | 122 | 6.046 | 10.394 | −13.628 | 1.00 | 49.67 | O |
| ATOM | 18772 | O | HOH | W | 123 | 49.689 | 38.586 | 48.909 | 1.00 | 55.60 | O |
| ATOM | 18775 | O | HOH | W | 124 | 45.493 | 54.453 | 19.719 | 1.00 | 56.14 | O |
| ATOM | 18778 | O | HOH | W | 125 | 53.857 | −1.594 | 17.970 | 1.00 | 47.50 | O |
| ATOM | 18781 | O | HOH | W | 126 | 37.535 | 64.000 | 25.273 | 1.00 | 57.85 | O |
| ATOM | 18784 | O | HOH | W | 127 | 13.976 | 22.044 | 60.248 | 1.00 | 40.96 | O |
| ATOM | 18787 | O | HOH | W | 128 | 23.599 | 74.068 | 17.026 | 1.00 | 75.23 | O |
| ATOM | 18790 | O | HOH | W | 129 | 18.700 | 59.069 | 48.419 | 1.00 | 50.89 | O |
| ATOM | 18793 | O | HOH | W | 130 | 37.126 | −0.022 | 8.701 | 1.00 | 45.81 | O |
| ATOM | 18796 | O | HOH | W | 131 | 35.158 | −8.435 | 3.593 | 1.00 | 49.91 | O |
| ATOM | 18799 | O | HOH | W | 132 | 7.279 | 46.170 | 47.118 | 1.00 | 67.83 | O |
| ATOM | 18802 | O | HOH | W | 133 | 38.706 | 45.973 | 60.559 | 1.00 | 62.46 | O |
| ATOM | 18805 | O | HOH | W | 134 | 3.810 | 8.884 | 19.812 | 1.00 | 56.91 | O |
| ATOM | 18808 | O | HOH | W | 135 | 30.111 | 19.847 | 28.143 | 1.00 | 63.17 | O |
| ATOM | 18811 | O | HOH | W | 136 | 21.897 | −24.638 | 9.123 | 1.00 | 52.08 | O |
| ATOM | 18814 | O | HOH | W | 137 | 44.787 | 29.869 | 37.519 | 1.00 | 57.52 | O |
| ATOM | 18817 | O | HOH | W | 138 | 16.457 | 45.882 | 14.500 | 1.00 | 50.23 | O |
| ATOM | 18820 | O | HOH | W | 139 | 6.610 | −9.081 | 1.907 | 1.00 | 66.14 | O |
| ATOM | 18823 | O | HOH | W | 140 | 30.624 | 17.120 | 56.530 | 1.00 | 43.89 | O |
| ATOM | 18826 | O | HOH | W | 141 | 24.663 | 54.575 | 7.459 | 1.00 | 75.45 | O |
| ATOM | 18829 | O | HOH | W | 142 | 3.291 | 53.721 | 16.115 | 1.00 | 52.15 | O |
| ATOM | 18832 | O | HOH | W | 143 | 17.798 | 44.614 | 43.412 | 1.00 | 35.28 | O |
| ATOM | 18835 | O | HOH | W | 144 | −3.893 | 63.767 | 39.732 | 1.00 | 57.61 | O |
| ATOM | 18838 | O | HOH | W | 145 | 26.346 | −12.103 | −11.462 | 1.00 | 51.74 | O |
| ATOM | 18841 | O | HOH | W | 146 | 25.178 | 37.664 | 46.309 | 1.00 | 42.85 | O |
| ATOM | 18844 | O | HOH | W | 147 | 10.226 | 70.895 | 7.418 | 1.00 | 23.44 | O |
| ATOM | 18847 | O | HOH | W | 148 | 47.426 | −13.956 | 31.608 | 1.00 | 57.62 | O |
| ATOM | 18850 | O | HOH | W | 149 | 42.632 | 40.326 | 64.290 | 1.00 | 46.68 | O |
| ATOM | 18853 | O | HOH | W | 150 | 47.965 | 16.027 | 69.814 | 1.00 | 52.99 | O |
| ATOM | 18856 | O | HOH | W | 151 | 35.258 | 17.505 | 50.695 | 1.00 | 37.18 | O |
| ATOM | 18859 | O | HOH | W | 152 | 42.765 | 25.171 | 73.864 | 1.00 | 45.61 | O |
| ATOM | 18862 | O | HOH | W | 153 | 17.682 | 37.118 | 60.178 | 1.00 | 48.19 | O |
| ATOM | 18865 | O | HOH | W | 154 | 4.917 | 51.651 | 20.340 | 1.00 | 59.26 | O |
| ATOM | 18868 | O | HOH | W | 155 | 18.284 | 5.065 | −22.455 | 1.00 | 66.44 | O |
| ATOM | 18871 | O | HOH | W | 156 | 48.174 | 37.200 | 72.008 | 1.00 | 51.47 | O |
| ATOM | 18874 | O | HOH | W | 157 | 8.994 | 39.168 | 22.488 | 1.00 | 59.94 | O |
| ATOM | 18877 | O | HOH | W | 158 | 38.685 | 8.951 | 71.019 | 1.00 | 53.48 | O |
| ATOM | 18880 | O | HOH | W | 159 | 43.966 | 16.431 | 65.023 | 1.00 | 57.15 | O |
| ATOM | 18883 | O | HOH | W | 160 | 45.628 | 3.345 | 77.459 | 1.00 | 52.32 | O |
| ATOM | 18886 | O | HOH | W | 161 | 49.305 | 24.574 | 45.364 | 1.00 | 73.80 | O |
| ATOM | 18889 | O | HOH | W | 162 | 23.302 | 22.739 | 1.013 | 1.00 | 64.58 | O |
| ATOM | 18892 | O | HOH | W | 163 | 4.901 | 31.457 | 31.801 | 1.00 | 63.61 | O |
| ATOM | 18895 | O | HOH | W | 164 | 35.668 | 9.733 | 38.971 | 1.00 | 57.20 | O |
| ATOM | 18898 | O | HOH | W | 165 | 50.588 | 20.385 | 73.103 | 1.00 | 65.81 | O |
| ATOM | 18901 | O | HOH | W | 166 | 27.437 | 13.505 | 15.130 | 1.00 | 54.05 | O |
| ATOM | 18904 | O | HOH | W | 167 | 18.140 | 44.428 | 69.479 | 1.00 | 60.04 | O |
| ATOM | 18907 | O | HOH | W | 168 | 20.428 | 61.835 | 21.571 | 1.00 | 50.20 | O |
| ATOM | 18910 | O | HOH | W | 169 | 16.346 | 20.786 | 61.900 | 1.00 | 68.58 | O |
| ATOM | 18913 | O | HOH | W | 170 | 10.022 | −16.733 | −3.299 | 1.00 | 45.71 | O |
| ATOM | 18916 | O | HOH | W | 171 | 52.508 | 6.294 | 75.257 | 1.00 | 53.77 | O |
| ATOM | 18919 | O | HOH | W | 172 | 58.749 | 6.101 | 66.320 | 1.00 | 46.84 | O |
| ATOM | 18922 | O | HOH | W | 173 | 28.460 | −0.709 | 0.570 | 1.00 | 49.98 | O |
| ATOM | 18925 | O | HOH | W | 174 | 10.291 | 60.292 | 8.525 | 1.00 | 55.76 | O |
| ATOM | 18928 | O | HOH | W | 175 | 4.095 | 2.945 | 15.154 | 1.00 | 58.94 | O |
| ATOM | 18931 | O | HOH | W | 176 | 7.244 | −13.384 | −5.254 | 1.00 | 48.74 | O |
| ATOM | 18934 | O | HOH | W | 177 | 32.377 | −16.618 | −4.059 | 1.00 | 44.28 | O |
| ATOM | 18937 | O | HOH | W | 178 | 47.649 | 0.734 | 67.335 | 1.00 | 49.13 | O |
| ATOM | 18940 | O | HOH | W | 179 | 12.789 | 47.261 | 47.980 | 1.00 | 63.48 | O |
| ATOM | 18943 | O | HOH | W | 180 | 5.446 | −15.480 | −4.436 | 1.00 | 57.50 | O |
| ATOM | 18946 | O | HOH | W | 181 | 9.087 | 37.020 | 24.095 | 1.00 | 67.94 | O |
| ATOM | 18949 | O | HOH | W | 182 | 20.864 | −8.162 | 29.795 | 1.00 | 49.49 | O |
| ATOM | 18952 | O | HOH | W | 183 | 43.991 | −1.427 | 62.252 | 1.00 | 55.02 | O |

Having thus described in detail preferred embodiments of the present invention, it is to be understood that the invention defined by the appended claims is not to be limited by particular details set forth in the above description as many apparent variations thereof are possible without departing from the spirit or scope thereof.

### REFERENCES

1. Acquati, F., Accarino, M., Nucci, C., Fumagalli, P., Jovine, L., Ottolenghi, S., Taramelli, R. The gene encoding DRAP (BACE2), a glycosylated transmembrane protein of the aspartic protease family, maps to the down critical region. FEBS Lett. 2000 Feb. 18; 468(1):59-64.
2. Anderson, J. P., Basi, G., Doane, M. T., Frigon, N., John, V., Power, M., Sinha, S., and Tatsuno, G., β-secretase enzyme compositions and methods. Patent # WO 00/27618, published Aug. 17, 2000.
3. Bennet, B. D., Denis, P., Haniu, M., Teplow, D. B., Kahn, S., Louis, J-C., Citron, M. And Vassar, R. A furin-like convertase mediates propeptide cleavage of BACE, the Alzheimer's β-secretase. J. Biol. Chem. 2000; 275, 37712-37717.
4. Bennett, B. D., Babu-Khan, S., Loeloff, R., Louis, J. C., Curran, E., Citron, M., Vassar R. Expression analysis of BACE2 in brain and peripheral tissues. J Biol Chem. 2000 Jul. 7; 275(27):20647-51.
5. Benjannet, S., Elagoz, A., Wickham, L., Mamarbachi, M., Munzer, J. S., Basak, A., Lazure, C, Cromlish, J. A., Sisodia, S., Checker, F., Chretien, M., Seidah, N. G. Post-translational processing of beta-secretase (beta-amyloid-converting enzyme) and its ectodomain shedding. The pro- and transmembrane/cytosolic domains affect its cellular activity and amyloid-beta production. J Biol Chem. 2001 Apr. 6; 276(14): 10879-87.
6. Blundell et al., 1976, Protein Crystallography, Academic Press.
7. Bigl, M., Apelt, J., Luschekina, E. A., Lange-Dohna, C., Rossner, S., Schliebs, R. Expression of beta-secretase mRNA in transgenic Tg2576 mouse brain with Alzheimer plaque pathology. Neurosci Lett. 2000 Oct. 6; 292(2):107-10.
8. Bodendorf, U., Fischer, F., Bodian, D., Multhaup, G., Paganetti, P. A splice variant of beta-secretase deficient in the amyloidogenic processing of the amyloid precursor protein. J Biol Chem. 2001 Apr. 13; 276(15):12019-23.
9. Bougueleret, L., Duclert, A., and Dumas Milne Edwards, J-P. Extended cDNAs for secreted proteins. Patent # WO 99/31236, published Jun. 24, 1999.
10. Cai, H., Wang, Y., McCarthy, D., Wen, H., Borchelt, D. R., Price D. L., Wong, P. C. BACE1 is the major 3-secretase for generation of Abeta peptides by neurons. Nat Neurosci. 2001 March; 4(3):233-4.
11. Capell A., Steiner, H., Willem, M., Kaiser, H., Meyer, C., Walter, J., Lammich, S., Multhaup, G., Haass, C. Maturation and pro-peptide cleavage of beta-secretase. J Biol Chem. 2000 Oct. 6; 275(40):30849-54.
12. Citron, M., and Bennet, B. Beta secretase genes and polypeptides. Patent # WO 00/58479, published Oct. 5, 2000.
13. Creemers, J. W. M., Dominguez, D. I., Plets, E., Serneels, L., Taylor, N. A., Multhaup, G., Craessaerts, K., Annaert, W. And De Strooper, B. Processing of β-secretase by furin and other members of the proprotein convertase family. J. Biol. Chem. 2000; 276, 4211-4217.
14. De Strooper, B. And Konig. G., Nature 1999; 402, 471-472.
15. Dominguez, D. I., De Strooper, B., and Annaert, W. Secretases as therapeutic targets for the treatment of Alzheimer's disease. Amyloid. 2001 June; 8(2):124-42.
16. Fairlie, D. P., Tyndall, J. D., Reid, R. C., Wong, A. K., Abbenante, G., Scanlon, M. J., March, D. R., Bergman, D. A., Chai, C. L., and Burjett, B. A. Conformational selection of inhibitors and substrates by proteolytic enzymes: implications for drug design and polypeptide processing. J. Med. Chem. 2000; 43(7):1271-81.
17. Farzan, M., Schnitzler, C. E., Vasilieva, N., Leung, D., and Choe H. Free in PMC, Related Articles BACE2, a beta-secretase homolog, cleaves at the beta site and within the amyloid-beta region of the amyloid-beta precursor protein. Proc Natl Acad Sci USA. 2000 Aug. 15; 97(17):9712-7.
18. Ghosh A. K, Shin D., Downs D., Koelsch G., Lin X., Ernolieff J. and Tang J. J. Am. Chem. Soc. 2000; 122 3522-3523.
19. Gurney, M. E., Bienkowski, M. H., Heinrikson, R. L., Parodi, L. A. and Yan, R. Alzheimers disease secretase. Patent # WO 00/17369, published Mar. 30, 2000.
20. Gurney, M., Bienkowski, M. J. Alzheimers disease secretase, APP substrates thereof, and uses therefor. Patent # WO 01/23533 A2, published Apr. 5, 2001.
21. Haniu, M., Denis, P., Young, Y., Mendiaz, E. A., Fuller, J., Hui, J. O., Bennet, B. D., Kahn, S., Ross, S., Burgess, T., Katta, V., Rogers, G., Vassar, R., and Citron, M. Characterization of Alzheimer's b-secretase protein BACE: A pepsin family member with unusual properties. J. Biol. Chem. 2000; 275, 21099-21106.
22. Hepburn, P. J., Griffiths, K., Harper, M. E. Angiogenic factors expressed by human prostatic cell lines: effect on endothelial cell growth in vitro. Prostate. 1997 Oct. 1; 33(2): 123-32.
23. Hong, L., Koelsch, G., Lin, X., Wu, S., Terzyan, S., Ghosh, A. K, Zhang, X. C. And Tang, J. Structure of the protease domain of memapsin 2 (β-secretase) complexed with inhibitor. Science, 2000; 290, 150-153.
24. Howlett, D. R., Simmons, D. L., Dingwall, C., and Christie, G. In search of an enzyme: the β-secretase of Alzheimers disease is an aspartic proteinase. Trends Neuroscience. 2000; 23, 565-570.
25. Huse, J. T. and Doms, R. W. Closing in on the amyloid cascade: recent insights into the cell biology of Alzheimer's disease. Mol Neurobiol. 2000 August-December; 22(1-3):81-98.
26. Huse, J. T., Pijak, D. S., Leslie, G. J., Lee, V. M., and Doms, R. W. Maturation and endosomal targeting of beta-site amyloid precursor protein-cleaving enzyme. The Alzheimer's disease beta-secretase. J Biol Chem. 2000 Oct. 27; 275(43):33729-37.
27. Hussain, I., Powell, D., Howlett, D. R., Tew, D. G., Meek, T. D., Chapman, C., Gloger, I. S., Murphy, K E, Southan, C. D., Ryan, D. M., Smith, T. S., Simmons, D. L., Walsh, F. S., Dingwall, C. And Christie, G. Identification of a novel aspartic protease (Asp2) as secretase. Mol. Cell. Neurosci. 1999; 14, 419-427.
28. Hussain, I, Christie, G., Schneider, K., Moore, S., Dingwall, C. Prodomain processing of asp1 (bace2) is autocatalytic. J Biol Chem. 2001 Jun. 29; 276(26):23322-8.
29. Hussain, I, Powell, D. J., Howlett, D. R., Chapman, G. A., Gilmour, L, Murdock, P. R., Tew, D. G., Meek, T. D., Chapman, C, Schneider, K, Ratcliffe, S. J., Tattersall, D., Testa, T. T., Southan, C., Ryan, D. M., Simmons, D. L., Walsh, F. S., Dingwall, C., Christie, G. ASP1 (BACE2) cleaves the amyloid precursor protein at the beta-secretase site. Mol Cell Neurosci. 2000 November; 16(5):609-19.
30. Irizarry, M. C., Locascio, J. J, Hyman, B. T. Beta-site APP cleaving enzyme mRNA expression in APP transgenic mice: anatomical overlap with transgene expression and static levels with aging. Am J Pathol. 2001 January; 158(1): 173-7.
31. Johnson et al., “Peptide Turn Mimetics,” In Biotechnology And Pharmacy, Pezzuto et aL, Eds., (Chapman and Hall, New York 1993).
32. Kahana, J. A new BACE is for therapy of Alzheimer's disease. Trends Cell Biol. 2001 May; 11(5):192.
33. King, L. A and Possee, R. D. The Baculovirus Expression System: A Laboratory Guide. 1992; Chapman and Hall.
34. Laprise, M. H., Grondin, F. and Dubois, C. M. Enhanced TGFβ1 maturation in High Five cells coinfected with recombinant baculovirus encoding the convertase furin/PACE: improved technology for the production of recombinant proproteins in insect cells. Biotech. Bioeng. 1998; 58, 85-91.
35. Lattman, Methods in Enzymology 1985 115:55-77.
36. Lee R J, Wang S, Low P S, Biochim Biophys Acta 1996 July 1312:237-42
37. Lin, X, Koelsch, G., Downs, D., Dashti, A. And Tang, J. Human aspartic protease memapsin 2 cleaves the β-secretase site of β-amyloid precursor protein. Proc. Natl. Acad. Sci USA 2000 97, 1456-1460.
38. Luo, Y., Bolon, M., Kahn, S., Bennet, B. D., Babu-Khan, S., Denis, P., Fan, W., Kha, H., Zhang, J., Gong, Y., Martin, L., Louis, J. C., Yan, Q., Richards, W. G., Citron, M., Vassar, R. Mice deficient in BACE1, the Alzheimer's beta-secretase, have normal phenotype and abolished beta-amyloid generation. Nat. Neurosci. 2001; 4(3):231-2.
39. Mallender, W., Yager, D., Onstead, L., Nichols, M. R., Eckman, C., Sambamurti, K, Kopcho, L. M., Marcinkeviciene, J., Copeland, R. A. and Rosenberry, T. L. Characterization of recombinant, soluble β-secretase from an insect cell expression system. Mol. Pharmacol. 2001; 59, 619-626.
40. Marcinkeviciene, J, Luo, Y, Graciani, N. R., Combs, A. P., and Copeland, R. A. Mechanism of Inhibition of beta-Site Amyloid Precursor Protein-cleaving Enzyme (BACE) by a Statine-based Peptide. J Biol Chem. 2001 Jun. 29; 276(26):23790-4.
41. Marcinkiewicz, M., and Seidah, N. G. Coordinated expression of beta-amyloid precursor protein and the putative beta-secretase BACE and alpha-secretase ADAM10 in mouse and human brain. J Neurochem. 2000 November; 75(5):2133-43.
42. Mazur, A., Wang, L., Anderson, M. E., Yee, R., Theres, H., Pearson, A., Olson, W., and Wathen, M. Functional similarity between electrograms recorded from an implantable cardioverter defibrillator emulator and the surface electrocardiogram. Pacing Clin Electrophysiol. 2001 January; 24(1):34-40.
43. Murphy, T., Yip, A., Brayne, C., Easton, D., Evans, J. G., Xuereb, J., Cairns, N., Esiri, M. M., and Rubinsztein, D. C. The BACE gene: genomic structure and candidate gene study in late-onset Alzheimer's disease. Neuroreport. 2001 Mar. 5; 12(3):631-4.
44. Navaza J. Acta Crystallogr. A. 1994; 50 157.
45. Nunan J. Small D H. Regulation of APP cleavage by alpha-, beta- and gamma-secretases. FEBS Lett. 2000 Oct. 13; 483(1):6-10.
46. Overly C C, Lee K D, Berthiaume E, Hollenbeck P J, Proc Natl Acad Sci USA 1995 April 92:3156-60
47. Potter H, and Dressier D. The potential of BACE inhibitors for Alzheimer's therapy. Nat Biotechnol. 2000 February; 18(2): 125-6.
48. Powell, D. J. Aspartic proteinase 2 (ASP2). Patent # EP 0855444 A2, published Jul. 27, 1998.
49. Pflugrath, J. W. Acta Crystallogr. D. 1999; 55 1718-1725.
50. Roberds, S. L., Anderson, J., Basi, G., Bienkowski, M. J., Branstetter, D. G., Chen, K. S., Freedman, S. B., Frigon, N. L., Games, D., Hu, K, Johnson-Wood, K, Kappenman, K E., Kawabe, T. T., Kola, I., Kuehn, R., Lee, M., Liu, W., Motter, R., Nichols, N. F., Power, M., Robertson, D. W., Schenk, D., Schoor, M., Shopp, G. M., Shuck, M. E., Sinha, S., Svensson, K. A., Tatsuno, G., Tintrup, H., Wijsman, J., and Wright, S., and McConlogue L. BACE knockout mice are healthy despite lacking the primary beta-secretase activity in brain: implications for Alzheimer's disease therapeutics. Hum Mol Genet. 2001 Jun. 1; 10(12):1317-24.
51. Robner, S., Apelt, J., Schliebs, R., Perez-Polo, J. R., and Bigl, V. Neuronal anf glial beta-secretase (BACE) protein expression in transgenic Tg2576 mice with amyloid plaque pathology. J. Neurosci. Res. 2001; 64(5):437-446.
52. Rossner, S., Apelt, J., Schliebs, R., Perez-Polo, J. R., and Bigl, V. Neuronal and glial beta-secretase (BACE) protein expression in transgenic Tg2576 mice with amyloid plaque pathology. J Neurosci Res. 2001 Jun. 1; 64(5):437-46.
53. Rossmann, 1972, “The Molecular Replacement Method,” Int. Sci. Rev. Ser. No. 13, Gordon & Breach, New York.
54. Russo, C., Schettini, G., Saido, T. C., Hulette, C., Lippa, C., Lannfelt, L., Ghetti, B., Gambetti, P., Tabaton, M., and Teller, J. K. Alzheimer's disease molecular consequences of presenilin-1 mutation. Nature. 2001 Jun. 7; 411(6838):655.
55. Satoh, J., Kuroda, Y. Amyloid precursor protein beta-secretase (BACE) mRNA expression in human neural cell lines following induction of neuronal differentiation and exposure to cytokines and growth factors. Neuropathology. 2000 December; 20(4):289-96.
56. Sauder, J. M., Arthur, J. W., and Dunbrack, R. L. Jr. Modeling of substrate specificity of the Alzheimer's disease amyloid precursor protein beta-secretase. J Mol Biol. 2000 Jul. 7; 300(2):241-8.
57. Sennvik, K., Benedikz, E., Fastbom, J., Sundstrom, E., and Winblad, B., Calcium ionophore A23187 specifically decreases the secretion of beta-secretase cleaved amyloid precursor protein during apoptosis in primary rat cortical cultures. J. Neurosci. 2001; 63(5):429-437.
58. Shi, X. P., Chen, E., Yin, K. C., Na, S., Garsky, V. M., Lai, M. T., Li, Y. M., Platchek, M., Register, R. B., Sardana, M. K., Tang, M. J., Thiebeau, J., Wood, T., Shafer, J. A., and Gardell, S. J. The pro domain of beta-secretase does not confer strict zymogen-like properties but does assist proper folding of the protease domain. J Biol Chem. 2001 Mar. 30; 276(13): 10366-73.
59. Sinha, S., Anderson, J. P., Barb ur, R., Basi, G. S., Caccavello, R., Davis, D., Doan, M., Dovey, H. F., Frigon, N., H ng, J., Jacobson-Croak, K, Jewett, N., Keim, P., Knops, J., Lieberburg, I., Power, M., Tan, H., Tatsuno, G, Tung, J., Schenk, D., Seubert, P., Suomensaari, S. M., Wand, S., Walker, D., Zhoa, J., Mcconlogue, L. and Varghese, J Nature 1999; 402 537-540
60. Solans, A., Estivill, X., and de La Luna, S. A new aspartyl protease on 21q22.3, BACE2, is highly similar to Alzheimer's amyloid precursor protein beta-secretase. Cytogenet Cell Genet. 2000; 89(34):177-84.
61. Tang, J. N., Hong, L., and Ghosh, A. Inhibitors of memapsin 2 and use thereof. Patent # WO01/00665 A2, published Jan. 4, 2001.
62. Tang, J. N., Lin, X., Koelsch, G. Catalytically active recombinant memapsin and methods of use thereof. Patent # WO 01/00663 A2, published Jan. 4, 2001.
63. Vassar, R. and Citron, M. Aβ-generating enzymes: recent advances in β- and γ-secretase research. Neuron, 2000; 7, 419-422
64. Vassar, R, Bennet, B. D., Babu-Khan, S., Kahn, S., Mendiaz, E. A., Denis, P., Teplow, D. B., Ross, S., Amarante, P., Loeloff, R., Luo, Y., Fisher, S., Fuller, J, Edenson, S., Lile, J., Jarosinski, M. A., Biere, A. L., Curran, E., Burgess, T., Louis, J-C., Collins, F., Treanor, J., Rogers, G. and Citron, M. β-secretase cleavage of Alzheimer's amyloid precursor protein by the transmembrane aspartic protease BACE. Science 1999; 286, 735-741.
65. Walter, J., Fluhrer, R., Hartung, B., Willem, M., Kaether, C., Capell, A., Lammich, S., Multhaup, G., and Haass, C. Phosphorylation regulates intracellular trafficking of beta-secretase. J Biol Chem. 2001 May 4; 276(18):14634-41.
66. Yan, R., Bienkowski, M. J., Shuck, M. E., Miao, H., Tory, M. C., Pauley, A. M., Brashier, Jr., Stratman, N. C., Mathews, W. R., Buhl, A. E., Carter, D. B., Tomasselli, A. G., Parodi, L. A., Heinrikson, R. J. and Gurney, M. E. Nature 1999; 402 533-537.
67. Yankner B. New clues to Alzheimer's disease: Unraveling the roles of anyloid and tau. Nat. Med. 1996; 2: 850-852.
68. Collaborative Computing Project, No. 4. The CCP4 Suite: Programs for Protein Crystallography, Acta Crystallographica, D50, 1994, 760-763.

1. A catalytic domain of BACE or a form of BACE that is suitable for crystallization with the correct disulphide bonding that eliminates the need for refolding and/or an apo-BACE crystal or an apo-BACE crystal that can be soaked to give complexes and/or a crystalline form of BACE having crystals that are grown at or near the physiological pH of the enzyme or between about pH 5.6 and about pH 5.8 and/or a BACE crystal having a space group of C2 and/or a BACE crystal having cell dimensions of a=236.63 Å or 236.63 Å±standard deviation (0.2 Å) or 236.63 Å+3.0 Å, b=105.02 Å or 105.02 Å±standard deviation (0.2 Å) or 105.02 Å±3.0 Å, and c=62.59 Å or 62.59 Å±standard deviation (0.2 Å) or 62.59 Å+3.0 Å and β=101.32° or 101.32°±standard deviation (0.2°) or between 101° and 108° with the asymmetric unit of the crystal containing three copies of BACE or cell dimensions a=238.3 Å or 238.3 Å±standard deviation (0.2 Å) or 238.3 Å±3.0 Å, b=107.4 Å±standard deviation (0.2 Å) or 107.4 Å±3.0 Å, and c=60.4 Å or 60.4 Å±standard deviation (0.2 Å) or 60.4 Å±3.0 Å and β=101.89° or 101.89°+standard deviation (0.2°) or between 101° and 108° and/or having an X-ray diffraction pattern corresponding to or resulting from any or all of the foregoing and/or having an X-ray diffraction pattern corresponding to or resulting from any or all of the foregoing; and/or having a space group transition from C2 to P2, together with an increase in the number of copies of the molecule in the asymmetric unit, while the cell dimensions and the packing of the P2, form are closely related to those of the C2 crystal form, on soaking the apo-BACE crystal with a ligand; and/or a BACE crystal having a resolution better than 3 Å; and/or a BACE crystal having the structure defined by the co-ordinates of Table 5.

2. A BACE crystal having the structure defined by the co-ordinates of Table 5.

3. An apo-BACE crystal grown at or near the physiological pH of the enzyme.

4. An apo-BACE crystal or an apo-BACE crystal that can be soaked to give complexes.

5. A crystalline form of BACE or a functional portion thereof having crystals that are grown at or near the physiological pH of the enzyme.

6. The crystalline form of BACE or functional portion thereof of claim 6 wherein the crystals are grown at a pH between about pH 5.6 and about pH 5.8.

7. A crystalline form of BACE or a functional portion thereof having a space group of C2 and cell dimensions of a=236.63 Å or 236.63 Å±standard deviation (0.2 Å) 236.63 Å±3.0 Å, b=105.02 Å or 105.02 Å±standard deviation (0.2 Å) or 105.02 Å+3.0 Å, and c=62.59 Å or 62.59 Å±standard deviation (0.2 Å) or 62.59 Å+3.0 Å and β=101.32° or 101.32°±standard deviation (0.2°) or between 101° and 108° with the asymmetric unit of the crystal containing three copies of BACE or cell dimensions a=238.3 Å or 238.3 Å±standard deviation (0.2 Å) or 238.3 Å±3.0 Å, b=107.4 Å or 107.4 Å±standard deviation (0.2 Å) or 107.4 Å+3.0 Å, and c=60.4 Å or 60.4 Å±standard deviation (0.2 Å) or 60.4 Å±3.0 Å and β=101.89° or 101.89°±standard deviation (0.2°) or between 101° and 108° and/or having an X-ray diffraction pattern corresponding to or resulting from any or all of the foregoing and/or having an X-ray diffraction pattern corresponding to or resulting from any or all of the foregoing and/or having a space group transition from C2 to P2₁ together with an increase in the number of copies of the molecule in the asymmetric unit, while the cell dimensions and the packing of the P2, form are closely related to those of the C2 crystal form, on soaking the apo-BACE crystal with a ligand.

8. A crystalline form of BACE or a functional portion thereof that has an active site containing one or more ligands other than the natural substrate or the substrate that occurs naturally or physiologically within the active site.

9. A method for ligand screening or identification comprising exposing the BACE crystals of any one of claims 2-8 to one or more test samples, and determining whether a ligand-BACE complex is formed.

10. The method of claim 9 wherein the BACE protein or functional portion thereof is exposed to the test samples by co-crystallizing the BACE protein or functional portion thereof in the presence of the one or more test samples.

11. The method of claim 9 wherein the BACE of claims 2-8 is soaked in a solution of one or more test samples.

12. A computer-assisted method for identifying or designing potential ligands to fit within the catalytic domain of BACE or a functional portion thereof:
comprising using a programmed computer comprising a processor, a data storage system, an input device, and an output device, the steps of: (a) inputting into the programmed computer through said input device data comprising the three-dimensional co-ordinates of a subset of the atoms in the BACE catalytic domain, optionally with structural information from ligand-BACE complexes, thereby generating a data set; (b) comparing, using said processor, said data set to a computer database of chemical structures stored in said computer data storage system; (c) selecting from said database, using computer methods, chemical structures having a portion that is structurally similar to said data set; (d) constructing, using computer methods, a model of a chemical structure having a portion that is structurally similar to said data set and (e) outputting to said output device the selected chemical structures having a portion similar to said data set; and optionally synthesizing one or more of the selected chemical structures; and further optionally contacting said synthesized selected chemical structure with BACE to ascertain whether said synthesized chemical structure is a ligand that fits within the catalytic domain of BACE and/or inhibits BACE; or,
comprising: providing the structure of BACE as defined by the co-ordinates of Table 5, providing the structure of a candidate modulator molecule, and fitting the structure of the candidate to the structure of the BACE of Table 5; or,
comprising: providing the co-ordinates of at least two atoms of Table 5 of BACE (“selected co-ordinates”), providing the structure of a candidate modulator molecule, and fitting the structure of the candidate to the selected co-ordinates of BACE; or,
comprising: providing the co-ordinates of at least a sub-domain of BACE, providing the structure of a candidate modulator molecule, and fitting the structure of the candidate to the sub-domain of BACE;
said method optionally further comprising: obtaining or synthesizing the chemical structure or candidate modulator and contacting the chemical structure or candidate modulator with BACE to determine the ability of the chemical structure or candidate to interact with BACE; or obtaining or synthesizing the chemical structure or candidate modulator and forming a complex of BACE and said chemical structure or candidate modulator, and analyzing the complex to determine the ability of said chemical structure or candidate modulator to interact with BACE.

13. A compound having a chemical structure selected using the methods of claims 9-12, said compound being a modulator of BACE.

14. A BACE protein or functional portion thereof comprising amino acid sequences of the catalytic domain that crystallize to the crystalline structure of claim 7, or to a structure that mimics that crystalline structure.

15. A BACE protein or functional portion thereof which, when compared to wild-type BACE or BACE of Genbank accession P56817 has one or more mutations or truncations to prevent glycosylation or facilitate crystallization and/or the growth of ordered, well-diffracting crystals.

16. The BACE protein or functional portion thereof of claim 15, which when compared with Genbank accession P56817 has one or more of: a mutation at amino acid (“aa”) 153, a mutation at aa 172, a mutation at aa 223, a mutation at aa 354, and one or more truncations.

17. The BACE protein or functional portion thereof of claim 16 wherein each of the mutations is asparagine to glutamine.

18. The BACE protein or functional portion thereof of claim 16 wherein the truncation results in a BACE extending from Thr 22 to Ser 453, with reference to Genbank Accession P56817.

19. The BACE protein or functional portion thereof of claim 16 wherein all of the mutations are present and each is asparagine to glutamine and there is a truncation resulting in a BACE extending from Thr 22 to Ser 453, with reference to Genbank Accession P56817.

20. The BACE protein or functional portion thereof of any one of claims 14-19 further including any one or more of: a tag to facilitate purification; a non-BACE signal sequence to facilitate or increase secretion of the protein into cell culture medium; and a tag to allow differentiation of species arising from incomplete pro-peptide cleavage.

21. The BACE protein or functional portion thereof of claim 20 wherein the tag to facilitate purification is a HIS tag, the non-BACE signal sequence is a baculovirus signal sequence, and the tag to allow differentiation of species is a FLAG tag.

22. The BACE protein or functional portion thereof of claim 21 wherein all of the tag to facilitate purification, the non-BACE signal sequence and the tag to allow differentiation are present.

23. A BACE protein or functional portion thereof containing any one or more of: a tag to facilitate purification; a non-BACE signal sequence to facilitate or increase secretion of the protein into cell culture medium; and a tag to allow differentiation of species arising from incomplete pro-peptide cleavage.

24. The BACE protein or functional portion thereof of claim 23 wherein the tag to facilitate purification is a HIS tag, the non-BACE signal sequence is a baculovirus signal sequence, and the tag to allow differentiation of species is a FLAG tag.

25. The BACE protein or functional portion thereof of claim 24 wherein all of the tag to facilitate purification, the non-BACE signal sequence and the tag to allow differentiation are present.

26. An isolated nucleic acid molecule encoding a BACE protein or functional portion thereof of any of claims 14-25 or a functional portion thereof.

27. The isolated nucleic acid molecule of claim 26 that has a reduced GC content via silent mutations from nucleotide sequences derived from wild-type BACE that would also encode the BACE protein.

28. A vector or cell comprising or expressing the nucleic acid molecule of claim 26.

29. A vector or cell comprising or expressing the nucleic acid molecule of claim 27.

30. The vector or cell of claim 28 which is a viral vector or a bacterial vector or a mammalian cell or a DNA plasmid.

31. The vector or cell of claim 29 which is a viral vector or a bacterial vector or a mammalian cell or a DNA plasmid.

32. The vector or cell of claims 30 or 31 which is a baculovirus vector or an insect cell.

33. The vector or cell of claim 26 further including a nucleic acid molecule encoding an enhancer that enhances in the particular vector or cell system the total amount of BACE produced and/or increases the fraction of processed protein.

34. The vector or cell of claim 27 further including a nucleic acid molecule encoding an enhancer that enhances in the particular vector or cell system the total amount of BACE produced and/or increases the fraction of processed protein.

35. The vector or cell of claims 33 or 34 wherein the enhancer is a prohormone convertase.

36. The vector or cell of claim 35 wherein the prohormone convertase is furin.

37. A vector or cell comprising a nucleic acid molecule encoding a BACE protein or functional portion thereof and a nucleic acid molecule encoding an enhancer that enhances in the particular vector or cell system the total amount of BACE produced and/or increases the fraction of processed protein.

38. The vector or cell of claim 37 wherein the enhancer is a prohormone convertase.

39. A kit for producing the vector or cell of claim 37 containing separately packaged nucleic acid molecules comprising (i) a BACE-protein encoding nucleic acid molecule and (ii) a nucleic acid molecule encoding the enhancer.

40. A method for obtaining a BACE protein comprising expressing a nucleic acid molecule according to any of claims 26 or 27 or the nucleic acid molecule of the vector or cell of any of claims 28 to 34 or 37.

41. A method for obtaining a BACE protein comprising expressing the nucleic acid molecule of the vector or cell of claim comprising expressing in a vector or cell the nucleic acid molecules of the kit of claim 39.

42. A method for crystallizing a BACE protein or functional portion thereof comprising dissolving a BACE protein according to any one of claims 14-25 in a suitable solvent and crystallizing the same either in the presence or absence of an inhibitor; wherein said method optionally further includes producing the BACE recombinantly or by expression thereof by a vector, recovering the BACE so produced, and growing crystals from the recovered BACE.

43. The method of claim 42 wherein the inhibitor is OM99-2.

44. A method for determining the crystal structure of a BACE protein or functional portion thereof comprising obtaining crystals of a BACE protein according to any one of claims 14-25 and obtaining an x-ray diffraction pattern thereof.

45. A method for ligand screening and design or identification comprising exposing the BACE crystals of a BACE protein or functional portion thereof to one or more test samples, and determining whether a ligand-BACE complex is formed; wherein the BACE or functional portion thereof has an unoccupied active site and is as claimed in any one of claims 5-8.

46. The method of claim 45 wherein the BACE is exposed to the test samples by either co-crystallizing the BACE or functional portion thereof in the presence of the one or more test samples or soaking the BACE or a functional portion thereof in a solution of one or more test samples.

47. A computer-assisted method for identifying or designing potential ligands to fit within the catalytic domain of BACE or a functional portion thereof:
comprising using a programmed computer comprising a processor, a data storage system, an input device, and an output device, the steps of: (a) inputting into the programmed computer through said input device data comprising the three-dimensional co-ordinates of a subset of the atoms in the BACE catalytic domain or functional portion thereof of any one of claims 5-8, optionally with structural information from ligand-BACE complexes, thereby generating a data set; (b) comparing, using said processor, said data set to a computer database of chemical structures stored in said computer data storage system; (c) selecting from said database, using computer methods, chemical structures having a portion that is structurally similar to said data set; (d) constructing, using computer methods, a model of a chemical structure having a portion that is structurally similar to said data set and (e) outputting to said output device the selected chemical structures having a portion similar to said data set; and optionally synthesizing one or more of the selected chemical structures; and further optionally contacting said synthesized selected chemical structure with BACE to ascertain whether said synthesized chemical structure is a ligand that fits within the catalytic domain of BACE and/or inhibits BACE; or,
comprising: providing the structure of BACE as defined by the co-ordinates of Table 5, providing the structure of a candidate modulator molecule, and fitting the structure of the candidate to the structure of the BACE of Table 5; or,
comprising: providing the co-ordinates of at least two atoms of Table 5 of BACE (“selected co-ordinates”), providing the structure of a candidate modulator molecule, and fitting the structure of the candidate to the selected co-ordinates of BACE; or,
comprising: providing the co-ordinates of at least a sub-domain of BACE, providing the structure of a candidate modulator molecule, and fitting the structure of the candidate to the sub-domain of BACE;
said method optionally further comprising: obtaining or synthesizing the chemical structure or candidate modulator and contacting the chemical structure or candidate modulator with BACE to determine the ability of the chemical structure or candidate to interact with BACE; or obtaining or synthesizing the chemical structure or candidate modulator and forming a complex of BACE and said chemical structure or candidate modulator, and analyzing the complex to determine the ability of said chemical structure or candidate modulator to interact with BACE.

48. A ligand identified in any of the methods of claims 45-47.

49. An assay comprising a BACE protein or functional portion thereof of any one of claims 14-25, and means to determine whether a compound is a modulator of BACE.

50. An antibody elicited by a BACE protein or functional portion thereof of any one of claims 14-25.

51. An inhibitor of a BACE protein or functional portion thereof of any one of claims 14-25.

52. A composition comprising the inhibitor of claim 51.

53. A composition comprising the ligand of claim 48.

54. A composition comprising the ligand of claim 13.

55. A composition comprising a product from the assay of claim 49.

56. A method for inhibiting BACE or the production of Aβ or fragments thereof or treating AD in an individual in need thereof comprising administering an inhibitor of a BACE protein or functional portion thereof as claimed in claim 51.

57. A method for inhibiting BACE or the production of Aβ or fragments thereof or treating AD in an individual in need thereof comprising administering a ligand of claim 13.

58. A method for inhibiting BACE or the production of Aβ or fragments thereof or treating AD in an individual in need thereof comprising administering a ligand of claim 48.

59. A BACE which comprises an amino acid sequence of SEQ ID 31 or an amino acid sequence having greater than 98.8% identity with SEQ ID 31.

60. The BACE of claim 59 having the amino acid sequence of SEQ ID 31.

61. A nucleic acid molecule encoding the BACE of claim 59 or 60.

62. An isolated nucleic acid molecule comprising a sequence of SEQ ID 32 or encoding a polypeptide sequence of SEQ ID 72 or a sequence having greater than 95.6% identity with SEQ ID 32 or the nucleic acid encoding a polypeptide sequence of SEQ ID 72.

63. The isolated nucleic acid molecule of claim 62 having the sequence of SEQ ID 32.

64. The isolated nucleic acid molecule of claim 63 encoding a polypeptide sequence of SEQ ID 72.

65. A vector or cell comprising the isolated nucleic acid molecule of any one of claims 62-64.

66. The vector or cell of claim 65 which is a baculovirus vector or an insect cell.

67. An inhibitor of the BACE of any one of claims 59 or 60.

68. An antibody elicited by the BACE of any one of claims 59 or 60.

69. A method for ligand screening and design or identification comprising exposing the BACE crystals of a BACE protein or functional portion thereof to one or more test samples, and determining whether a ligand-BACE complex is formed; wherein the BACE or functional portion thereof has an unoccupied active site and is as claimed in any one of claims 59 or 60.

70. The method of claim 69 wherein the BACE is exposed to the test samples by either co-crystallizing the BACE or functional portion thereof in the presence of the one or more test samples or soaking the BACE or a functional portion thereof in a solution of one or more test samples.

71. A computer-assisted method for identifying or designing potential ligands to fit within the catalytic domain of BACE or a functional portion thereof:
comprising using a programmed computer comprising a processor, a data storage system, an input device, and an output device, the steps of: (a) inputting into the programmed computer through said input device data comprising the three-dimensional co-ordinates of a subset of the atoms in the BACE catalytic domain or functional portion thereof of any one of claims 59 or 60, optionally with structural information from ligand-BACE complexes, thereby generating a data set; (b) comparing, using said processor, said data set to a computer database of chemical structures stored in said computer data storage system; (c) selecting from said database, using computer methods, chemical structures having a portion that is structurally similar to said data set; (d) constructing, using computer methods, a model of a chemical structure having a portion that is structurally similar to said data set and (e) outputting to said output device the selected chemical structures having a portion similar to said data set; and optionally synthesizing one or more of the selected chemical structures; and further optionally contacting said synthesized selected chemical structure with BACE to ascertain whether said synthesized chemical structure is a ligand that fits within the catalytic domain of BACE and/or inhibits BACE; or,
comprising: providing the structure of BACE as defined by the co-ordinates of Table 5, providing the structure of a candidate modulator molecule, and fitting the structure of the candidate to the structure of the BACE of Table 5; or,
comprising: providing the co-ordinates of at least two atoms of Table 5 of BACE (“selected co-ordinates”), providing the structure of a candidate modulator molecule, and fitting the structure of the candidate to the selected co-ordinates of BACE; or,
comprising: providing the co-ordinates of at least a sub-domain of BACE, providing the structure of a candidate modulator molecule, and fitting the structure of the candidate to the sub-domain of BACE;
said method optionally further comprising: obtaining or synthesizing the chemical structure or candidate modulator and contacting the chemical structure or candidate modulator with BACE to determine the ability of the chemical structure or candidate to interact with BACE; or obtaining or synthesizing the chemical structure or candidate modulator and forming a complex of BACE and said chemical structure or candidate modulator, and analyzing the complex to determine the ability of said chemical structure or candidate modulator to interact with BACE.

72. A ligand identified in any of the methods of claims 68-71.

73. An assay comprising a BACE protein or functional portion thereof of any one of claims 58 or 59, and means to determine whether a compound is a modulator of BACE.

74. A composition comprising the inhibitor of claim 67.

75. A composition comprising the ligand of claim 72.

76. A composition comprising a product from the assay of claim 73.

77. A method for inhibiting BACE or the production of Aβ or fragments thereof or treating AD in an individual in need thereof comprising administering an inhibitor of a BACE protein or functional portion thereof as claimed in claim 67.

78. A method for inhibiting BACE or the production of Aβ or fragments thereof or treating AD in an individual in need thereof comprising administering a ligand of claim 72.

79. Use of an inhibitor of a BACE protein or functional portion thereof as claimed in claim 51 for preparing a composition or medicament for inhibiting BACE or the production of Aβ or fragments thereof or treating AD in an individual in need thereof.

80. Use of an inhibitor of a BACE protein or functional portion thereof as claimed in claim 13 for preparing a composition or medicament for inhibiting BACE or the production of Aβ or fragments thereof or treating AD in an individual in need thereof.

81. Use of an inhibitor of a BACE protein or functional portion thereof as claimed in claim 48 for preparing a composition or medicament for inhibiting BACE or the production of Aβ or fragments thereof or treating AD in an individual in need thereof.

82. Use of an inhibitor of a BACE protein or functional portion thereof as claimed in claim 51 for use in therapy.

83. Use of an inhibitor of a BACE protein or functional portion thereof as claimed in claim 67 for preparing a composition or medicament for inhibiting BACE or the production of Aβ or fragments thereof or treating AD in an individual in need thereof.

84. Use of an inhibitor of a BACE protein or functional portion thereof as claimed in claim 72 for preparing a composition or medicament for inhibiting BACE or the production of Aβ or fragments thereof or treating AD in an individual in need thereof.

85. A computer system for generating structures or performing rational compound or drug design for BACE or complexes of BACE with a potential modulator, the system containing either: atomic co-ordinate data according to Table 5, said data defining the three-dimensional structure of BACE or at least one sub-domain thereof, or structure factor data for BACE, said structure factor data being derivable from the atomic co-ordinate data of Table 5.

86. A computer readable media with either: atomic co-ordinate data according to Table 5, said data defining the three-dimensional structure of BACE or at least one sub-domain thereof, or structure factor data for BACE, said structure factor data being derivable from the atomic co-ordinate data of Table 5.

87. A method of doing business comprising providing to a user the computer system of claim 85 or the computer readable media of claim 83 or the three-dimensional structure of BACE or at least one sub-domain thereof, or structure factor data for BACE, said structure factor data being derivable from the atomic co-ordinate data of Table 5.


SEQUENCE LISTING (72 sequences)

SEQ ID NO: 1 - DNA, 2526 bp, Homo sapiens
ccacgcgtccgcagcccgcccgggagctgcgagccgcgagctggattatggtggcctgagcagccaacgcagccgcaggagcccggagcccttgcccctgcccgcgccgccgcccgccggggggaccagggaagccgccaccggcccgccatgcccgcccctcccagccccgccgggagcccgcgcccgctgcccaggctggccgccgccgtgccgatgtagcgggctccggatcccagcctctcccctgctcccgtgctctgcggatctcccctgaccgctctccacagcccggacccgggggctggcccagggccctgcaggccctggcgtcctgatgcccccaagctccctctcctgagaagccaccagcaccacccagacttgggggcaggcgccagggacggacgtgggccagtgcgagcccagagggcccgaaggccggggcccaccatggcccaagccctgccctggctcctgctgtggatgggcgcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtgaggaggcccatgggcagaagatagagattcccctggaccacacctccgtggttcactttggtcacaagtaggagacacagatggcacctgtggccagagcacctcaggaccctccccacccaccaaatgcctctgccttgatggagaaggaaaaggctggcaaggtgggttccagggactgtacctgtaggaaacagaaaagagaagaaagaagcactctgctggcgggaatactcttggtcacctcaaatttaagtcgggaaattctgctgcttgaaacttcagccctgaacctttgtccaccattcctttaaattctccaacccaaagtattcttcttttcttagtttcagaagtactggcatcacacgcaggttaccttggcgtgtgtccctgtggtaccctggcagagaagagaccaagcttgtttccctgctggccaaagtcagtaggagaggatgcacagtttgctatttgctttagagacagggactgtataaacaagcctaacattggtgcaaagattgcctcttgaattaaaaaaaaaaactagaaaaaaaaaaaaaaa

SEQ ID NO: 2 - DNA, 2054 bp, Homo sapiens
atggcccaagccctgccctggctcctgctgtggatgggcgcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtcatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtgaggaggcccatgggcagaagatagagattcccctggaccacacctccgtggttcactttggtcacaagtaggagacacagatggcacctgtggccagagcacctcaggaccctccccacccaccaaatgcctctgccttgatggagaaggaaaaggctggcaaggtgggttccagggactgtacctgtaggaaacagaaaagagaagaaagaagcactctgctggcgggaatactcttggtcacctcaaatttaagtcgggaaattctgctgcttgaaacttcagccctgaacctttgtccaccattcctttaaattctccaacccaaagtattcttcttttcttagtttcagaagtactggcatcacacgcaggttaccttggcgtgtgtccctgtggtaccctggcagagaagagaccaagcttgtttccctgctggccaaagtcagtaggagaggatgcacagtttgctatttgctttagagacagggactgtataaacaagcctaacattggtgcaaagattgcctcttgaattaaaaaaaaaaa

SEQ ID NO: 3 - DNA, 16 bp, Homo sapiens
aaaaaaaaaaaaaaaa

SEQ ID NO: 4 - DNA, 1506 bp, Homo sapiens
atggcccaagccctgccctggctcctgctgtggatgggcgcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtga

SEQ ID NO: 5 - DNA, 4 bp, Homo sapiens
atgc

SEQ ID NO: 6 - DNA, 6 bp, Homo sapiens
tactag

SEQ ID NO: 7 - DNA, 5 bp, Homo sapiens
taaat

SEQ ID NO: 8 - DNA, 8 bp, Homo sapiens
cagtcaca

SEQ ID NO: 9 - DNA, 10 bp, Homo sapiens
ccaaggcttc

SEQ ID NO: 10 - DNA, 9 bp, Homo sapeins
aataaggaa

SEQ ID NO: 11 - DNA, 5 bp, Homo sapiens
cacac

SEQ ID NO: 12 - DNA, 4 bp, Homo sapiens
aagc

SEQ ID NO: 13 - DNA, 17 bp, Homo sapiens
aagatggtaagcgctat

SEQ ID NO: 14 - DNA, 9 bp, Homo sapiens
tgttttata

SEQ ID NO: 15 - DNA, 13 bp, Homo sapiens
tgcttttggcagc

SEQ ID NO: 16 - DNA, 5 bp, Homo sapiens
agctg

SEQ ID NO: 17 - DNA, 4 bp, Homo sapiens
ctca

SEQ ID NO: 18 - DNA, 5 bp, Homo sapiens
ttctg

SEQ ID NO: 19 - DNA, 13 bp, Homo sapiens
cctttgctgcgga

SEQ ID NO: 20 - DNA, 8 bp, Homo sapiens
tccgaatt

SEQ ID NO: 21 - DNA, 15 bp, Homo sapiens
cagactacaaggacg

SEQ ID NO: 22 - DNA, 4 bp, Homo sapiens
atga

SEQ ID NO: 23 - DNA, 4 bp, Homo sapiens
caag

SEQ ID NO: 24 - DNA, 1296 bp, Homo sapiens
acccagcacgggatccggctgcctctgcgcagcggcctgggaggagctccactgggactgcggctgcctcgagagaccgacgaagagcctgaggagcctggacggagaggcagctttgtggagatggtggacaacctgagaggcaagtcaggacagggctactacgtggagatgaccgtgggcagccctcctcagacgctcaacatcctggtggatacaggcagcagtaactttgcagtgggtgctgcaccacacccattcctgcatcgctactaccagaggcagctgtccagcacataccgagacctacggaagggtgtgtatgtgccctacacccagggcaagtgggaaggagagctgggcaccgacctggtaagcatcccccatggacctcaagtcactgtgcgtgccaacattgctgccatcactgaatcagacaagttcttcatccaaggctccaactgggaaggcatcctggggctggcctatgctgagattgccaggcctgacgactccctggagcctttctttgactctctggtaaagcagacccacgttcccaacctcttctccctgcagctttgtggtgctggcttccctctccaacagtctgaagtgctggcctctgtcggagggagcatgatcattggaggtatcgaccactcgctgtacacaggcagtctctggtatacacccatccgacgagagtggtattatgaggtgatcattgtgcgagtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttacccaacagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggaccttttgtcaccttggacatggaagactgtggctacaatattccacagacagatgagtca

SEQ ID NO: 25 - DNA, 6 bp, Homo sapiens
catcat

SEQ ID NO: 26 - DNA, 5 bp, Homo sapiens
cacca

SEQ ID NO: 27 - DNA, 8 bp, Homo sapiens
ccatcact

SEQ ID NO: 28 - protein, 501 aa, Homo sapiens
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 29 - protein, 408 aa, Homo sapiens
ETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPQVTVRANIAAITESDKFFIQGSNWEGILGLAYAEIARPDDSLEPFFDSLVKQTHVPNLFSLQLCGAGFPLQQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTQQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDES

SEQ ID NO: 30 - protein, 6 aa, Homo sapiens
HHHHHH

SEQ ID NO: 31 - protein, 414 aa, Homo sapiens
ETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPQVTVRANIAAITESDKFFIQGSNWEGILGLAYAEIARPDDSLEPFFDSLVKQTHVPNLFSLQLCGAGFPLQQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTQQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESHHHHHH

SEQ ID NO: 32 - DNA, 1470 bp, Homo sapiens
atgctactagtaaatcagtcacaccaaggcttcaataaggaacacacaagcaagatggtaagcgctattgttttatatgtgcttttggcagcagctgctcattctgcctttgctgcggatccgaattcagactacaaggacgacgatgacaagacccagcacgggatccggctgcctctgcgcagcggcctgggaggagctccactgggactgcggctgcctcgagagaccgacgaagagcctgaggagcctggacggagaggcagctttgtggagatggtggacaacctgagaggcaagtcaggacagggctactacgtggagatgaccgtgggcagccctcctcagacgctcaacatcctggtggatacaggcagcagtaactttgcagtgggtgctgcaccacacccattcctgcatcgctactaccagaggcagctgtccagcacataccgagacctacggaagggtgtgtatgtgccctacacccagggcaagtgggaaggagagctgggcaccgacctggtaagcatcccccatggacctcaagtcactgtgcgtgccaacattgctgccatcactgaatcagacaagttcttcatccaaggctccaactgggaaggcatcctggggctggcctatgctgagattgccaggcctgacgactccctggagcctttctttgactctctggtaaagcagacccacgttcccaacctcttctccctgcagctttgtggtgctggcttccctctccaacagtctgaagtgctggcctctgtcggagggagcatgatcattggaggtatcgaccactcgctgtacacaggcagtctctggtatacacccatccgacgagagtggtattatgaggtgatcattgtgcgagtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttacccaacagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggaccttttgtcaccttggacatggaagactgtggctacaatattccacagacagatgagtcacatcatcaccaccatcactaa

SEQ ID NO: 33 - DNA, 5 bp, Homo sapiens
atggc

SEQ ID NO: 34 - DNA, 17 bp, Homo sapiens
agccctgccctggctcc

SEQ ID NO: 35 - DNA, 8 bp, Homo sapiens
tgctgtgg

SEQ ID NO: 36 - DNA, 8 bp, Homo sapiens
atgggcgc

SEQ ID NO: 37 - DNA, 6 bp, Homo sapiens
gggagt

SEQ ID NO: 38 - DNA, 5 bp, Homo sapiens
gctgc

SEQ ID NO: 39 - DNA, 6 bp, Homo sapiens
ctgccc

SEQ ID NO: 40 - DNA, 5 bp, Homo sapiens
acggc

SEQ ID NO: 41 - DNA, 2357 bp, Homo sapiens
acccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgagccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtgaggaggcccatgggagaaagatagagattcccctgggaccacacctccgtggttcactttggtcacaagtaggagacacagatggcacctgtggccagagcacctcaggaccctccccacccaccaaatgcctctgccttgatggagaaggaaaaggctggcaaggtgggttccagggactgtacctgtaggaaacagaaaagagaagaaagaagcactctgctggcgggaatactcttggtcacctcaaatttaagtcgggaaattctgctgcttgaaacttcagccctgaacctttgtccaccattcctttaaattctccaacccaaagtattcttcttttcttagtttcagaagtactggcatcacacgcaggttaccttggcgtgtgtccctgtggtacccgggcagagaagagaccaagcttgtttccctgctggccaaagtcagtaggagaggatgcacagtttgctatttgctttagagacagggactgtataaacaagcctaacattggtgcaaagattgcctcttgaattaaaaaaaaaaactagattgactatttatacaaatgggggcggctggaaagaggagaaggagagggagtacaaagacagggaatagtgggatcaaagctaggaaaggcagaaacacaaccactcaccagtcctagttttagacctcatctccaagatagcatcccatctcagaagatgggtgttgttttcaatgttttcttttctgtggttgcagcctgaccaaaagtgagatgggaagggcttatctagccaaagagctcttttttagctctcttaaatgaagtgcccactaaggaagttccacttgaacacatggaatttctgccatattaatttccattgtctctatctggaaccaccctttaatctctacata

SEQ ID NO: 42 - DNA, 8 bp, Homo sapiens
ttaggtcc

SEQ ID NO: 43 - DNA, 10 bp, Homo sapiens
agcacttgaa

SEQ ID NO: 44 - DNA, 4 bp, Homo sapiens
aata

SEQ ID NO: 45 - DNA, 7 bp, Homo sapiens
ttcctaa

SEQ ID NO: 46 - DNA, 80 bp, Homo sapiens
  misc_feature (3)..(4): Wherein n is a, t, c, or g.
ccnnaatttgncttgggggctttgcngnccaggtgctaaaagggnttgggtaggngnccncttntatntnatncctnaaa

SEQ ID NO: 47 - DNA, 8 bp, Homo sapiens
  misc_feature (6)..(7): Wherein n is a, t, c, or g.
ggttanng

SEQ ID NO: 48 - DNA, 6 bp, Homo sapiens
gggagt

SEQ ID NO: 49 - DNA, 5 bp, Homo sapiens
gctgc

SEQ ID NO: 50 - DNA, 6 bp, Homo sapiens
ctgccc

SEQ ID NO: 51 - DNA, 5 bp, Homo sapiens
acggc

SEQ ID NO: 52 - DNA, 1478 bp, Homo sapiens
acccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtgaggaggcccatgggcagaagatagagattcccctgg

SEQ ID NO: 53 - DNA, 510 bp, Homo sapiens
accacacctccgtggttcactttggtcacaagtaggagacacagatggcacctgtggccagagcacctcaggaccctccccacccaccaaatgcctctgccttgatggagaaggaaaaggctggcaaggtgggttccagggactgtacctgtaggaaacagaaaagagaagaaagaagcactctgctggcgggaatactcttggtcacctcaaatttaagtcgggaaattctgctgcttgaaacttcagccctgaacctttgtccaccattcctttaaattctccaacccaaagtattcttcttttcttagtttcagaagtactggcatcacacgcaggttaccttggcgtgtgtccctgtggtaccctggcagagaagagaccaagcttgtttccctgctggccaaagtcagtaggagaggatgcacagtttgctatttgctttagagacagggactgtataaacaagcctaacattggtgcaaagattgcctcttgaattaaaaaaaa

SEQ ID NO: 54 - DNA, 284 bp, Homo sapiens
actagattgactatttatacaaatgggggcggctggaaagaggagaaggagagggagtacaaagacagggaatagtgggatcaaagctaggaaaggcagaaacacaaccactcaccagtcctagttttagacctcatctccaagatagcatcccatctcagaagatgggtgttgttttcaatgttttcttttctgtggttgcagcctgaccaaaagtgagatgggaagggcttatctagccaaagagctcttttttagctctcttaaatgaagtgcccactaag

SEQ ID NO: 55 - DNA, 11 bp, Homo sapiens
aagttccactt

SEQ ID NO: 56 - DNA, 8 bp, Homo sapiens
aacacatg

SEQ ID NO: 57 - DNA, 21 bp, Homo sapiens
aatttctgccatattaatttc

SEQ ID NO: 58 - DNA, 14 bp, Homo sapiens
attgtctctatctg

SEQ ID NO: 59 - DNA, 181 bp, Homo sapiens
aaccaccctttattctacatatgataggcagcactgaaatatcctaaccccctaagctccaggtgccctgtgggagagcaactggactatagcagggctgggctctgtcttcctggtcataggctcactctttcccccaaatcttcctctggagctttgcagccaaggtgctaaaaggaat

SEQ ID NO: 60 - DNA, 721 bp, Homo sapiens
aggtaggagacctcttctatctaatccttaaaagcataatgttgaacattcattcaacagctgatgccctataacccctgcctggatttcttcctattaggctataagaagtagcaagatctttacataattcagagtggtttcattgccttcctaccctctctaatggcccctccatttatttgactaaagcatcrcacagtggcactagcattataccaagagtatgagaaatacagtgctttatggctctaacattactgccttcagtatcaaggctgcctggagaaaggatggcagcctcagggcttccttatgtcctccaccacaagagctccttgatgaaggtcatctttttcccctatcctgttcttcccctccccgctcctaatggtacgtgggtacccaggctggttcttgggctaggtagtggggaccaagttcattacctccctatcagttctagcatagtaaactacggtaccagtgttagtgggaagagctgggttttcctagtatacccactgcatcctactcctacctggtcaacccgctgcttccaggtatgggacctgctaagtgtggaattacctgataagggagagggaaatacaaggagggcctctggtgttcctggcctcagccagctgcccmcaagccataaaccaataaamcaagaatactgagtctaaaaaaaaaaaaaaaaaaaaaaaaaaaaaaa

SEQ ID NO: 61 - DNA, 1296 bp, Homo sapiens
actcagcatggtattcgtctgccactgcgtagcggtctgggtggtgctccactgggtctgcgtctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcacctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtcatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtca

SEQ ID NO: 62 - DNA, 1449 bp, Homo sapiens
atgctactagtaaatcagtcacaccaaggcttcaataaggaacacacaagcaagatggtaagcgctattgttttatatgtgcttttggcagcagctgctcattctgcctttgctgcggatccgaattcagactacaaggacgacgatgacaagacccagcacgggatccggctgcctctgcgcagcggcctgggaggagctccactgggactgcggctgcctcgagagaccgacgaagagcctgaggagcctggacggagaggcagctttgtggagatggtggacaacctgagaggcaagtcaggacagggctactacgtggagatgaccgtgggcagccctcctcagacgctcaacatcctggtggatacaggcagcagtaactttgcagtgggtgctgcaccacacccattcctgcatcgctactaccagaggcagctgtccagcacataccgagacctacggaagggtgtgtatgtgccctacacccagggcaagtgggaaggagagctgggcaccgacctggtaagcatcccccatggacctcaagtcactgtgcgtgccaacattgctgccatcactgaatcagacaagttcttcatccaaggctccaactgggaaggcatcctggggctggcctatgctgagattgccaggcctgacgactccctggagcctttctttgactctctggtaaagcagacccacgttcccaacctcttctccctgcagctttgtggtgctggcttccctctccaacagtctgaagtgctggcctctgtcggagggagcatgatcattggaggtatcgaccactcgctgtacacaggcagtctctggtatacacccatccgacgagagtggtattatgaggtgatcattgtgcgagtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttacccaacagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggaccttttgtcaccttggacatggaagactgtggctacaatattccacagacagatgagtca

SEQ ID NO: 63 - DNA, 4 bp, Homo sapiens
catc

SEQ ID NO: 64 - DNA, 5 bp, Homo sapiens
atcac

SEQ ID NO: 65 - DNA, 5 bp, Homo sapiens
cacca

SEQ ID NO: 66 - DNA, 5 bp, Homo sapiens
tcact

SEQ ID NO: 67 - protein, 453 aa, Homo sapiens
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLHLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDES

SEQ ID NO: 68 - protein, 5 aa, Homo sapiens
HHHHH

SEQ ID NO: 69 - protein, 461 aa, Homo sapiens
LRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 70 - protein, 408 aa, Homo sapiens; identical to SEQ ID NO: 29

SEQ ID NO: 71 - protein, 5 aa, Homo sapiens
HHHHH

SEQ ID NO: 72 - protein, 459 aa, Homo sapiens
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLHLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESHHHHHH